(19) United States
(12) Patent Application Publication
(10) Pub. No.: US 2025/0161277 A1
(43) Pub. Date: May 22, 2025

(54) COMPOSITIONS AND METHODS OF MODULATING SHORT-CHAIN DEHYDROGENASE ACTIVITY

(71) Applicants: RODEO THERAPEUTICS CORPORATION, Thousand Oaks, CA (US); CASE WESTERN RESERVE UNIVERSITY, Cleveland, OH (US); BOARD OF REGENTS OF THE UNIVERSITY OF TEXAS SYSTEM, Austin, TX (US)

(72) Inventors: Sanford Markowitz, Pepper Pike, OH (US); Joseph Ready, Carrollton, TX (US); Stephen L. Gwaltney, II, Chapel, NC (US); Monika Antczak, Fort Worth, TX (US)

(21) Appl. No.: 19/029,244

(22) Filed: Jan. 17, 2025

Related U.S. Application Data
(62) Division of application No. 17/296,055, filed on May 21, 2021.
(60) Provisional application No. 62/770,571, filed on Nov. 21, 2018.

(51) Int. Cl. A61K 31/4365; A61K 31/437; A61K 31/501; A61K 31/506; A61K 31/519; A61K 31/5377; A61K 31/541; A61K 45/06; C07D 495/04; C07D 519/00

(52) U.S. Cl. CPC A61K 31/4365; A61K 31/437; A61K 31/501; A61K 31/506; A61K 31/519; A61K 31/5377; A61K 31/541; A61K 45/06; C07D 495/04; C07D 519/00

(57) ABSTRACT

Compounds and methods of modulating 15-PGDH activity, modulating tissue prostaglandin levels, treating disease, diseases disorders, or conditions in which it is desired to modulate 15-PGDH activity and/or prostaglandin levels include 15-PGDH inhibitors described herein.

### CROSS-REFERENCE TO RELATED APPLICATIONS

This application is a divisional of U.S. patent application Ser. No. 17/296,055, filed May 21, 2021, which is a national stage application under 35 U.S.C. § 371 of International Application No. PCT/US2019/062686, filed Nov. 21, 2019, which claims the benefit of priority to U.S. Provisional Application No. 62/770,571, filed Nov. 21, 2018. Each of the foregoing applications is incorporated herein by reference in its entirety.

### BACKGROUND

Short-chain dehydrogenases (SCDs) are a family of dehydrogenases that share only 15% to 30% sequence identity, with similarity predominantly in the coenzyme binding domain and the substrate binding domain. In addition to their role in detoxification of ethanol, SCDs are involved in synthesis and degradation of fatty acids, steroids, and some prostaglandins, and are therefore implicated in a variety of disorders such as lipid storage disease, myopathy, SCD deficiency, and certain genetic disorders.

The SCD, 15-hydroxy-prostaglandin dehydrogenase (15-PGDH), (hydroxyprostaglandin dehydrogenase 15-(nicotinamide adeninedinucleotide); 15-PGDH; Enzyme Commission number 1.1.1.141; encoded by the HPGD gene), represents the key enzyme in the inactivation of a number of active prostaglandins, leukotrienes and hydroxyeicosatetraenoic acids (HETEs) (e.g., by catalyzing oxidation of PGE₂ to 15-keto-prostaglandin E2, 15k-PGE). The human enzyme is encoded by the HPGD gene and consists of a homodimer with subunits of a size of 29 kDa. The enzyme belongs to the evolutionarily conserved superfamily of short-chain dehydrogenase/reductase enzymes (SDRs), and according to the recently approved nomenclature for human enzymes, it is named SDR36C1. Thus far, two forms of 15-PGDH enzyme activity have been identified, NAD+-dependent type I 15-PGDH that is encoded by the HPGD gene, and the type II NADP-dependent 15-PGDH, also known as carbonyl reductase 1 (CBR1, SDR21C1). However, the preference of CBR1 for NADP and the high Km values of CBR1 for most prostaglandin suggest that the majority of the in vivo activity can be attributed to type I 15-PGDH encoded by the HPGD gene, that hereafter, and throughout all following text, simply denoted as 15-PGDH.

Recent studies suggest that inhibitors of 15-PGDH and activators of 15-PGDH could be therapeutically valuable. It has been shown that there is an increase in the incidence of colon tumors in 15-PGDH knockout mouse models. A more recent study implicates increased 15-PGDH expression in the protection of thrombin-mediated cell death. It is well known that 15-PGDH is responsible for the inactivation of prostaglandin E2 (PGE₂), which is a downstream product of COX-2 metabolism. PGE₂ has been shown to be beneficial in a variety of biological processes, such as hair density, dermal wound healing, and bone formation.

### SUMMARY

Embodiments described herein relate to compounds and methods of modulating short chain dehydrogenase (SCD) (e.g., 15-PGDH) activities, modulating tissue prostaglandin levels, and/or treating diseases, disorders, or conditions in which it is desired to modulate SCD (e.g., 15-PGDH) activity and/or prostaglandin levels.

In some embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having a structure of formula (IA):

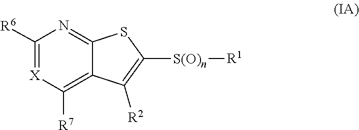

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³.
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
R¹⁰ is —OH, halogen, alkyl, or alkoxy;
X is N or CH;
m is 0, 1, or 2;
n is 0, 1, or 2; and
t is 0, 1, or 2.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In still other embodiments, R² is —NH₂.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₆ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₆ haloalkyl, 3- to 6-membered cycloalkyl, phenyl, 5- to 10-membered heteroaryl each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In other embodiments, the compound has the structure of formula (II):

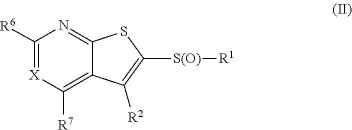

or a pharmaceutically acceptable salt, tautomer, or solvate thereof, wherein:
R¹ is 3- to 6-membered cycloalkyl, —(C₁-C₆ alkylene)-(3- to 6-membered cycloalkyl), 3- to 6-membered heterocyclyl, or —(C₁-C₆ alkylene)-(3- to 6-membered heterocyclyl);
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is oxo, halogen, —CN, —N(R⁵)₂, —OH, —O-alkylene-OH,—S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
X is N or CH;
m is 0, 1, or 2; and
t is 0, 1, or 2.

In some embodiments, R¹ is 3- to 5-membered cycloalkyl or —(C₁-C₆ alkylene)-(3- to 5-membered cycloalkyl).

In other embodiments, R¹ is cyclobutyl.

In still other embodiments, R¹ is a bicyclic 4- to 6-membered cycloalkyl.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In other embodiments, R⁷ is C₁-C₃ alkyl, C₁-C₃ haloalkyl, 3-membered cycloalkyl, phenyl, 4-membered heterocyclyl, or 5- to 6-membered heteroaryl, each of which is optionally substituted with one or more R⁴. In other embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, isopropyl, cyclopropyl, phenyl, pyridyl, or triazole, each of which is optionally substituted with one or more R⁴. In other embodiments, R⁷ is —CF₃, isopropyl, cyclopropyl, phenyl, pyridyl, or triazole, each of which is optionally substituted with one or more R⁴.

In some embodiments, R⁷ is —CF₃, isopropyl,

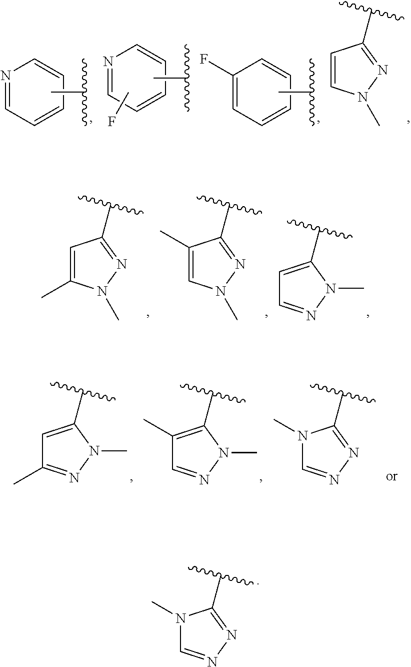

In other embodiments, R⁷ is —CF₃, isopropyl,

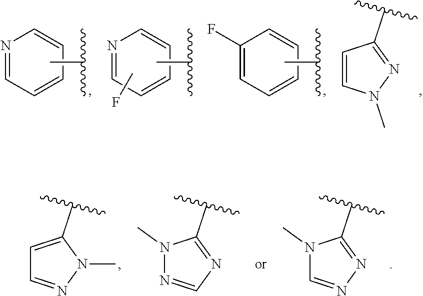

In still other embodiments, R⁷ is —CF₃,

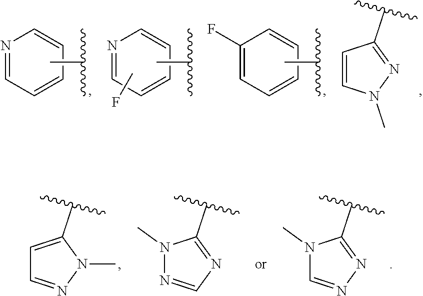

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In other embodiments, the compound has the structure of formula (III):

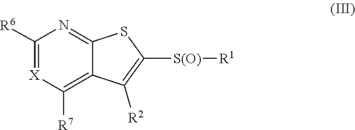

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is C₁-C₆ haloalkyl, aryl or heteroaryl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
each R⁵ is independently, H, alkyl, -alkylene-OH optionally substituted with —OH, -alkylene-NH₂, -alkylene-N(R′)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁴ is halogen, alkyl, or alkoxy;
X is N or CH; and
m is 0, 1, or 2.

In some embodiments, R¹ is cyclobutyl. In some embodiments, R¹ is —(C₁-C₄ alkylene)—(C₁-C₃ alkoxy).

In some embodiments, R⁷ is —CF₃, pyridyl, pyrazole, phenyl, or triazole, each of which is optionally substituted with one or more R⁴.

In other embodiments, R⁷ is —CF₃, pyridyl, fluorophenyl, or a triazole optionally substituted with one or more halogen or methyl.

In still other embodiments, R⁷ is —CF₃,

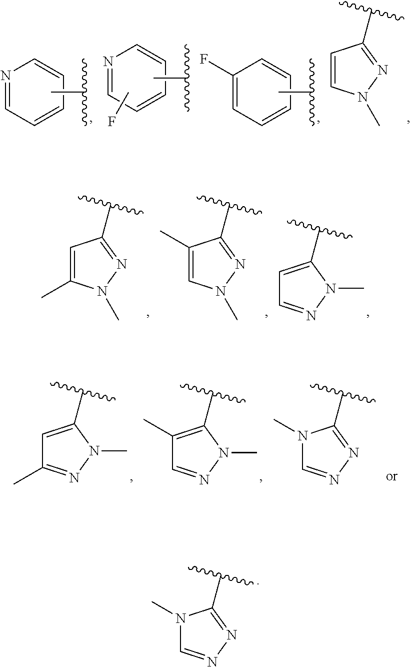

In still other embodiments, R⁷ is —CF₃.

In still other embodiments, R⁷ is

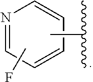

In some embodiments, R⁷ is

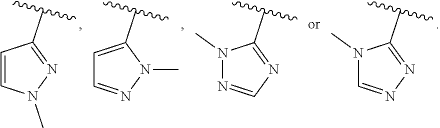

In other embodiments, R⁶ is 8- to 10-membered bicyclic heteroaryl optionally substituted with one or more R³.

In other embodiments, the compound has the structure of formula (IV):

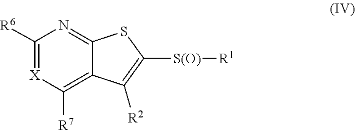

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is optionally substituted with one or more R³;
R⁷ is 3- to 6-membered cycloalkyl, optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is halogen, —CN, —NH₂, —OH, or C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R⁷ is cyclopropyl.

In other embodiments, R¹ is 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In some embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R¹ is cyclobutyl. In some embodiments, R¹ is —(C₁-C₄ alkylene)—(C₁-C₃ alkoxy).

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In other embodiments, the compound has the structure of formula (V):

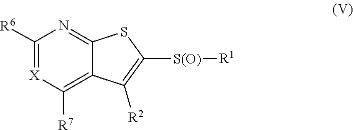

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is substituted with one or more R³;
R⁷ is haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl);
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently H, C₁-C₆ alkyl, —(C₁-C₆ alkylene)-OH optionally substituted with —OH, -alkylene-NH₂, -alkylene-N(R⁹)₂, -alkylene-O-alkylene-OH, -alkylene-O-alkylene-NH₂, —C(O)(C₁-C₆ alkyl), —C(O)O(C₁-C₆ alkyl), alkylene-COOH, or —S(O)ₘ(C₁-C₆ alkyl);
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R¹ is cyclobutyl. In some embodiments, —(C₁-C₄ alkylene)—(C₁-C₃ alkoxy).

In some embodiments, R³ is —O—(C₁-C₆ alkylene)-N(R⁵)₂, —N(R⁵)₂ or —N(R⁵)(C₁-C₆ alkylene-OH).

In other embodiments, R⁵ is H, C₁-C₆ alkyl, —(C₁-C₆ alkylene)-OH, or —S(O)₂(C₁-C₃ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In still other embodiments, R³ is —NHCH₂CH₂OH.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In some embodiments, R⁶ is furan, thiophene, pyrrole, thiazole, isothiazole, oxazole, isooxazole, pyrazole, imidazole, triazole, pyridine, pyrimidine, pyridazine, or pyrazine, each optionally substituted with one or more R³.

In other embodiments, R⁶ is thiazole, imidazole, oxazole, pyridine, or pyrimidine.

In some embodiments, R⁶ is 8- to 10-membered bicyclic heteroaryl optionally substituted with one or more R³.

In other embodiments, R⁶ is 5- to 6-membered heterocyclyl, optionally substituted with one or more R³, selected from morpholine, pyridine-one, or piperidine.

In some embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, cyclopropyl, phenyl, pyrazole, pyridyl, or triazole, each of which is optionally substituted with one or more R⁴. In still other embodiments, R⁷ is —CF₃,

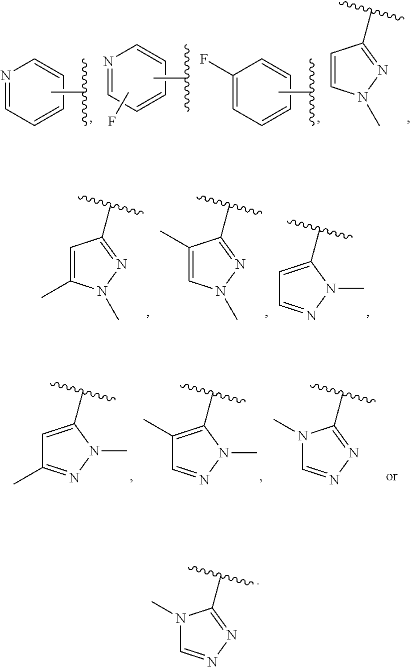

In some embodiments, the compound has the structure of formula (VI):

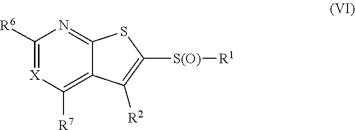

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is fused bicyclic heterocyclyl or fused bicyclic heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-O-alkylene-OH, -alkylene-O-alkylene-NH₂, —C(O)-alkyl, —C(O)O-alkyl, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH; and
m is 0, 1, or 2.

In some embodiments, R⁶ is 8- to 10-membered fused bicyclic heteroaryl, each of which is optionally substituted with one or more R.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₃ alkyl, C₁-C₆ haloalkyl, 3- to 6-membered cycloalkyl, phenyl, 3- to 6-membered heterocyclyl, or 5- to 6-membered heteroaryl, each of which is optionally substituted with one or more R⁴.

In other embodiments, R⁷ is C₁-C₃ alkyl, C₁-C₆ haloalkyl, 3- to 6-membered cycloalkyl, phenyl, pyrazole, pyridyl, or triazole, each of which is optionally substituted with one or more R⁴. In other embodiments, R⁷ is C₁-C₆ haloalkyl, 3- to 6-membered cycloalkyl, phenyl, pyrazole, pyridyl, or triazole, each of which is optionally substituted with one or more R⁴.

In other embodiments, R⁷ is —CF₃, isopropyl, cyclopropyl, phenyl, pyridyl, or triazole, each of which is optionally substituted with one or more R⁴. In other embodiments, R⁷ is —CF₃, cyclopropyl, phenyl, pyridyl, or triazole, each of which is optionally substituted with one or more R⁴. In further embodiments, R⁷ is isopropyl,

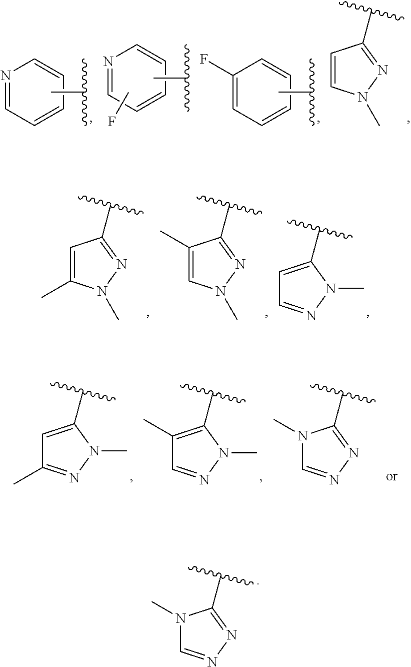

In further embodiments, R⁷ is

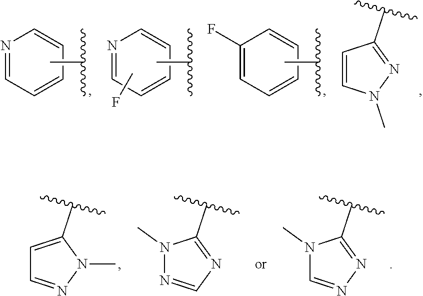

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3. In some embodiments, R¹ is cyclobutyl. In some embodiments, —(C₁-C₄ alkylene)—(C₁-C₃ alkoxy).

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In some embodiments, R³ is —NHCH₂CH₂OH or —N(CH₃)CH₂CH₂OH.

In other embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, the compound has the structure of formula (VII):

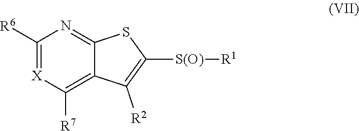

or a pharmaceutically acceptable salt, tautomer, or solvate thereof, wherein:
R¹ is cyclobutyl or —(C₁-C₄ alkylene)—(C₁-C₃ alkoxy);
R² is —NH₂, CN, or —NHC(O)alkyl;
R⁶ is heterocyclyl or heteroaryl, each of which is optionally substituted with one or more R³.
R⁷ is —CF₃, isopropyl,

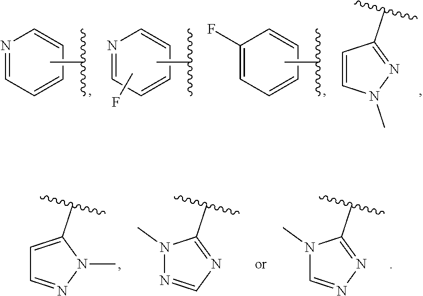

R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
R¹⁰ is —OH, halogen, alkyl, or alkoxy;
X is N or CH;
m is 0, 1, or 2;
p is 0 or 1; and
t is 0, 1, or 2.

In some embodiments, the compound has the structure of formula (VIII):

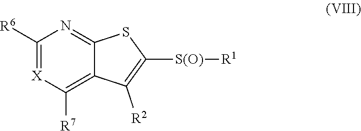

or a pharmaceutically acceptable salt, tautomer, or solvate thereof, wherein:
R¹ is cyclobutyl or —(C₁-C₄ alkylene)—(C₁-C₃ alkoxy);
R² is —NH₂, CN, or —NHC(O)alkyl;
R⁶ is

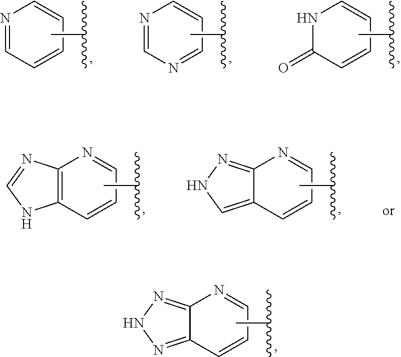

each of which is optionally substituted with one or more R³;
R⁷ is —CF₃, isopropyl, cyclopropyl, cyclobutyl,

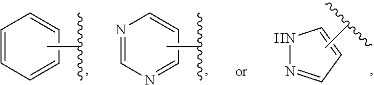

each of which is optionally substituted with one or more R⁴;
R³ is —NH₂, —NH(C₁-C₃ alkyl), —NH(C₁-C₄ alkylene)-OH, or C₁-C₃ alkyl;
R⁴ is C₁-C₃ alkyl; and
X is N or CH.

In some embodiments, the compound or 15-PGDH inhibitor can inhibit the enzymatic activity of recombinant 15-PGDH at an IC₅₀ of less than 1 μM, at an IC₅₀ of less than 250 nM, at an IC₅₀ of less than 50 nM, at an IC₅₀ of less than 10 nM, at an IC₅₀ of less than 5 nM at a recombinant, at an IC₅₀ of about 2.5 nM to about 10 nM, or less than about 2.5 nM at a 15-PGDH concentration of about 5 nM to about 10 nM.

The 15-PGDH inhibitor can be provided in a topical composition that can be applied to skin of a subject to promote and/or stimulate pigmentation of the skin and/or hair growth and/or inhibiting hair loss, and/or treat skin damage or inflammation.

The 15-PGDH inhibitor can also be administered to a subject to promote wound healing, tissue repair, and/or tissue regeneration and/or engraftment or regeneration of a tissue graft.

In one embodiment, the 15-PGDH inhibitor can be administered to a subject to treat at least one of oral ulcers, gum disease, colitis, ulcerative colitis, gastrointestinal ulcers, inflammatory bowel disease, vascular insufficiency, Raynaud's disease, Buerger's disease, diabetic neuropathy, pulmonary artery hypertension, cardiovascular disease, and renal disease.

In another embodiment, the 15-PGDH inhibitor can be administered to a subject in combination with a prostanoid agonist for the purpose of enhancing the therapeutic effect of the agonist in prostaglandin responsive conditions.

In other embodiments, the 15-PGDH inhibitor can be administered to a subject and/or tissue of the subject to increase tissue stem cells. For example, the 15-PGDH inhibitor can be administered to bone marrow of a subject to increase stem cells in the subject.

In still other embodiments, the 15-PGDH inhibitor can be administered to a tissue graft donor, bone marrow graft donor, and/or a hematopoietic stem cell donor, and/or a tissue graft, and/or a bone marrow graft, and/or a hematopoietic stem cell graft, to increase the fitness of a donor tissue graft, a donor bone marrow graft, and/or a donor hematopoietic stem cell graft. In one embodiment, the 15-PGDH inhibitor is administered ex vivo to a tissue graft, and/or a bone marrow graft, and/or a hematopoietic stem cell graft. For example, the 15-PGDH inhibitor can be administered to a subject, and/or bone marrow of a subject to increase the fitness of the marrow as a donor graft, and/or to a preparation of hematopoietic stem cells of a subject to increase the fitness of the stem cell preparation as a donor graft, and/or to a preparation of peripheral blood hematopoietic stem cells of a subject to increase the fitness of the stem cell preparation as a donor graft, and/or to a preparation of umbilical cord blood stem cells to increase the fitness of the stem cell preparation as a donor graft, and/or to a preparation of umbilical cord blood stem cells to decrease the number of units of umbilical cord blood required for transplantation.

In other embodiments, the 15-PGDH inhibitor can be administered to a subject to mitigate tissue graft rejection, to enhance tissue and/or bone marrow graft engraftment, to enhance bone marrow graft engraftment, following treatment of the subject or the marrow of the subject with radiation therapy, chemotherapy, or immunosuppressive therapy, to enhance engraftment of a progenitor stem cell graft, hematopoietic stem cell graft, or an umbilical cord blood stem cell graft, to enhance engraftment of a hematopoietic stem cell graft, or an umbilical cord stem cell graft, following treatment of the subject or the marrow of the subject with radiation therapy, chemotherapy, or immunosuppressive therapy, and/or in order to decrease the number of units of umbilical cord blood required for transplantation into the subject.

In other embodiments, the 15-PGDH inhibitor can be administered to a recipient of a tissue graft transplant, bone marrow transplant, and/or hematopoietic stem cell transplant, or of an umbilical cord stem cell transplant, in order to decrease the administration of other treatments or growth factors.

In some embodiments, the 15-PGDH inhibitor can be administered to a subject or to a tissue graft of a subject to mitigate graft rejection, to enhance graft engraftment, and/or to enhance graft engraftment following treatment of the subject or the marrow of the subject with radiation therapy, chemotherapy, or immunosuppressive therapy.

In other embodiments, the 15-PGDH inhibitor can be administered to a subject or to the bone marrow of a subject to confer resistance to toxic or lethal effects of exposure to radiation, to confer resistance to the toxic effect of Cytoxan, the toxic effect of fludarabine, the toxic effect of chemotherapy, or the toxic effect of immunosuppressive therapy, to decrease pulmonary toxicity from radiation, and/or to decrease infection.

In still other embodiments, the 15-PGDH inhibitor can be administered to a subject to increase neutrophil counts following a hematopoetic cell transplant with bone marrow, hematopoetic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoetic cell transplant with bone marrow, hematopoetic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoetic cell transplant with bone marrow, hematopoetic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoetic cell transplant with bone marrow, hematopoetic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In other embodiments, the administration of a 15-PGDH inhibitor can be used to modulate hematopoietic stem cells and hematopoiesis. For example, a 15-PGDH inhibitor can be administered alone or in combination with a cytokine to a subject in need thereof to increase and/or mobilize hematopoiectic stem cells and/or neutrophils in the blood, marrow, and/or tissue of the subject.

In some embodiments, the administration of a 15-PGDH inhibitor can be in combination with G-CSF for the purpose of increasing neutrophils.

In other embodiments, the administration of a 15-PGDH inhibitor can be in combination with a hematopoietic cytokine for the purpose of increasing neutrophils.

In still other embodiments, the administration of a 15-PGDH inhibitor can be in combination with G-CSF for the purpose of increasing numbers of and/or of mobilizing peripheral blood hematopoietic stem cells.

In other embodiments, the administration of a 15-PGDH inhibitor can be in combination with a hemopoietic cytokine for the purpose of increasing numbers of and/or of mobilizing peripheral blood hematopoietic stem cells.

In some embodiments, the administration of a 15-PGDH inhibitor can be in combination with a second agent, including, for example, Plerixafor, for the purpose of increasing numbers of and/or of mobilizing peripheral blood hematopoietic stem cells.

In other embodiments, the administration of a 15-PGDH inhibitor can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor can be in combination with a hemopoietic cytokine for the purpose of increasing numbers of and/or of mobilizing peripheral blood hematopoietic stem cells for use in hematopoietic stem cell transplantation.

In other embodiments, the administration of a 15-PGDH inhibitor can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor can be in combination with G-CSF for the purpose of increasing numbers of hematopoietic stem cells in blood or bone marrow.

In other embodiments, the administration of a 15-PGDH inhibitor can be in combination with a hemopoietic cytokine for the purpose of increasing numbers of hematopoietic stem cells in blood or bone marrow.

In other embodiments, the 15-PGDH inhibitor can be administered to a subject and/or tissue of the subject to increase tissue stem cells. For example, the 15-PGDH inhibitor can be administered to bone marrow of a subject to increase stem cells in the subject.

In other embodiments, the 15-PGDH inhibitor can be administered to a recipient of a tissue graft transplant, bone marrow transplant, and/or hematopoietic stem cell transplant, or of an umbilical cord stem cell transplant, in order to decrease the administration of other treatments or growth factors.

In still other embodiments, the 15-PGDH inhibitor can be administered to a subject to increase neutrophil counts following a hematopoetic cell transplant with bone marrow, hematopoetic stem cells, or umbilical cord blood, to increase neutrophil counts in a subject with neutropia following chemotherapy administration or radiation therapy, to increase neutrophil counts in a subject with aplastic anemia, myelodysplasia, myelofibrosis, neutropenia due to other bone marrow diseases, drug induced neutropenia, autoimmune neutropenia, idiopathic neutropenia, or neutropenia following viral infections, to increase neutrophil counts in a subject with neutropia, to increase platelet counts following a hematopoetic cell transplant with bone marrow, hematopoetic stem cells, or umbilical cord blood, to increase platelet counts in a subject with thrombocytopenia following chemotherapy administration or radiation therapy, to increase platelet counts in a subject with aplastic anemia, myelodysplasia, myelofibrosis, thrombocytopenia due to other bone marrow diseases, drug induced thrombocytopenia, autoimmune thrombocytopenia, idiopathic thrombocytopenic purpura, idiopathic thrombocytopenia, or thrombocytopenia following viral infections, to increase platelet counts in a subject with thrombocytopenia, to increase red blood cell counts, or hematocrit, or hemoglobin level, following a hematopoetic cell transplant with bone marrow, hematopoetic stem cells, or umbilical cord blood, to increase red blood cell counts, or hematocrit, or hemoglobin level in a subject with anemia following chemotherapy administration or radiation therapy, to increase red blood cell counts, or hematocrit, or hemoglobin level counts in a subject with aplastic anemia, myelodysplasia, myelofibrosis, anemia due to other disorder of bone marrow, drug induced anemia, immune mediated anemias, anemia of chronic disease, anemia following viral infections, or anemia of unknown cause, to increase red blood cell counts, or hematocrit, or hemoglobin level in a subject with anemia, to increase bone marrow stem cells, following a hematopoetic cell transplant with bone marrow, hematopoetic stem cells, or umbilical cord blood, to increase bone marrow stem cells in a subject following chemotherapy administration or radiation therapy, and/or to increase bone marrow stem cells in a subject with aplastic anemia, myelodysplasia, myelofibrosis, other disorder of bone marrow, drug induced cytopenias, immune cytopenias, cytopenias following viral infections, or cytopenias.

In other embodiments, the 15-PGDH inhibitor can be administered to a subject to increase responsiveness to cytokines in the presence of cytopenias, with cytopenias including any of: neutropenia, thrombocytopenia, lymphocytopenia and anemia; and with cytokines having increased responsiveness potentiated by the 15-PGDH inhibitor including any of: G-CSF, GM-CSF, EPO, IL-3, IL-6, TPO, TPO-RA (thrombopoietin receptor agonist), and SCF.

In some embodiments, the 15-PGDH inhibitor can be administered to a subject to increase bone density, treat osteoporosis, promote healing of fractures, or promote healing after bone surgery or joint replacement and/or to promote healing of bone to bone implants, bone to artificial implants, dental implants, and bone grafts.

In other embodiments, the 15-PGDH inhibitor can be administered to a subject or to the intestine of a subject to increase stem cells or cell proliferation in the intestine and/or and confer resistance to toxic or lethal effects of exposure to radiation or the toxic, lethal, or mucositis effects resultant from treatment with chemotherapy.

In some embodiments, the 15-PGDH inhibitor can be administered to a subject or to intestine of a subject as a treatment for colitis, ulcerative colitis, or inflammatory bowel disease.

In other embodiments, the 15-PGDH inhibitor can be administered to a subject to increase liver regeneration following liver surgery, following live liver donation, following liver transplantation, or following liver injury by toxins and/or to promote recovery from or resistance to liver toxins, including acetaminophen and related compounds.

In still other embodiments, the 15-PGDH inhibitor can be administered to a subject to treat erectile dysfunction.

In yet other embodiments, the 15-PGDH inhibitor can be administered to inhibit at least one of the growth, proliferation, or metastasis of 15-PGDH expressing cancers.

Still other embodiments described herein relate to a method of treating a subject in need of cell therapy. The method includes administering to the subject a therapeutically effective amount of a preparation comprising human hematopoietic stem cell administered a 15-PGDH inhibitor described herein and/or a therapeutic composition comprising human hematopoietic stem cells and a 15-PGDH inhibitor described herein.

In some embodiments, the subject has received human hematopoietic stem cells and/or has received the preparation and/or the therapeutic composition.

In other embodiments, the subject has acute myelogenous leukemia (AML), acute lymphoblastic leukemia (ALL), chronic myelogenous leukemia (CML), chronic lymphocytic leukemia (CLL), juvenile myelomonocytic leukemia, Hodgkin's lymphoma, non-Hodgkin's lymphoma, multiple myeloma, severe aplastic anemia, Fanconi's anemia, paroxysmal nocturnal hemoglobinuria (PNH), pure red cell aplasia, amegakaryocytosis/congenital thrombocytopenia, severe combined immunodeficiency syndrome (SCID), Wiskott-Aldrich syndrome, beta-thalassemia major, sickle cell disease, Hurler's syndrome, adrenoleukodystrophy, metachromatic leukodystrophy, myelodysplasia, refractory anemia, chronic myelomonocytic leukemia, agnogenic myeloid metaplasia, familial erythrophagocytic lymphohistiocytosis, solid tumors, chronic granulomatous disease, mucopolysaccharidoses, or Diamond Blackfan anemia.

Other embodiments relate to a method of treating a subject having at least one symptom associated with an ischemic tissue or a tissue damaged by ischemia. The method includes administering to the subject a therapeutically effective amount of a preparation comprising human hematopoietic stem cells administered a 15-PGDH inhibitor described herein and/or a therapeutic composition comprising human hematopoietic stem cells and a 15-PGDH inhibitor described herein.

In some embodiments, the ischemia can be associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient isc hemic attack (TIA), traumatic brain injury, ulcerative colitis, vascular disease of the kidney, vascular inflammatory conditions, von Hippel-Lindau syndrome, and wounds to tissues or organs.

Other embodiments relate to methods for treating and/or preventing fibrosis and various fibrotic diseases, disorders or conditions by administration of 15-PGDH inhibitors. In some embodiments, a 15-PGDH inhibitor described herein can be administered to a subject in need thereof to decrease fibrotic symptoms, such as collagen deposition, inflammatory cytokine expression, and inflammatory cell infiltration, and treat and/or prevent various fibrotic diseases, disorders, and conditions characterized, in whole or in part, by the excess production of fibrous material, including excess production of fibrotic material within the extracellular matrix, or the replacement of normal tissue elements by abnormal, non-functional, and/or excessive accumulation of matrix-associated components.

Fibrotic diseases, disorders and conditions characterized, in whole or in part, by excess production of fibrotic material can include systemic sclerosis, multifocal fibrosclerosis, nephrogenic systemic fibrosis, scleroderma (including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulomanry fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis, acute fibrosis, organ specific fibrosis, and the like.

In some embodiments, a method of treating or preventing a fibrotic disease, disorder or condition includes administering to a subject in need thereof a therapeutically effect amount of a 15-PGDH inhibitor.

In some embodiments, the 15-PGDH inhibitors can be used to treat or prevent lung fibrosis. Lung fibrosis, which can be treated, can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

In other embodiments, the 15-PGDH inhibitors can be used to treat or prevent kidney fibrosis. The kidney fibrosis can result from dialysis following kidney failure, catheter placement, a nephropathy, glomerulosclerosis, glomerulonephritis, chronic renal insufficiency, acute kidney injury, end stage renal disease or renal failure, or combinations thereof.

In other embodiments, the 15-PGDH inhibitors can be used to treat or prevent liver fibrosis. The liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, alpha-1-antitrypsin deficiency, primary biliary cirrhosis, drug reaction and exposure to toxins, or combinations thereof.

In some embodiments, the 15-PGDH inhibitors can be used to treat or prevent heart fibrosis, for example, cardiac fibrosis and endomyocardial fibrosis.

In some embodiments, the 15-PGDH inhibitors can be used to treat or prevent systemic sclerosis.

In some embodiments, the 15-PGDH inhibitors can be used to treat or prevent fibrotic diseases, disorders or conditions caused by post-surgical adhesion formation.

In some embodiments, the 15-PGDH inhibitors can be used for reducing or preventing scar formation in a subject.

In other embodiments, the 15-PGDH inhibitors can be used to reduce or prevent scar formation on skin or scleroderma.

In various embodiments, the 15-PGDH inhibitors can be administered at a therapeutically effective amount such that at least one symptom or feature of a fibrotic disease, disorder or condition, or other related diseases, disorders or conditions, is reduced in intensity, severity, or frequency, or has delayed onset.

In other embodiments, the 15-PGDH inhibitors can be used in a method for decreasing or reducing collagen secretion or collagen deposition in a tissue or organ, such as the lung, the liver, the intestines, the colon, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult can be organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

Other embodiments described herein relate to the use of 15-PGDH inhibitors in combination with corticosteroids or TNF inhibitors to treat inflammation, reduce aberrant activity of the immune system, and/or promote wound healing in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory, disorders, immune disorders, and/or wounds by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects.

In some embodiments, the inflammatory and/or immune disease or disorder treated with the combination of 15-PGDH inhibitor and a corticosteroid or TNF inhibitor can include intestinal, gastrointestinal, or bowel disorders. As described below, it was found that inhibitors of short-chain dehydrogenase activity, such as 15-PGDH inhibitors, can be administered to a subject in need thereof alone or in combination with corticosteroids and tumor necrosis factor (TNF)-alpha antagonists to treat intestinal, gastrointestinal, or bowel disorders, such as oral ulcers, gum disease, gastritis, colitis, ulcerative colitis, gastric ulcers, inflammatory bowel disease, and Crohn's disease.

In other embodiments, the 15-PGDH inhibitor can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. For example, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids.

The 15-PGDH inhibitor can also be administered in combination with a corticosteroid or TNF inhibitor to a subject to promote wound healing, tissue repair, and/or tissue regeneration and/or engraftment or regeneration of a tissue graft.

In some embodiments, the 15-PGDH inhibitor can be administered to a subject at an amount effective to increase prostaglandin levels in the subject and attenuate corticosteroid induced adverse and/or cytotoxic effects.

### DETAILED DESCRIPTION

While the following terms are believed to be well understood by one of ordinary skill in the art, the following definitions are set forth to facilitate explanation of the presently disclosed subject matter.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

The term “pharmaceutically acceptable salts” include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods. The term “pharmaceutically acceptable salts” also includes those obtained by reacting the active compound functioning as an acid, with an inorganic or organic base to form a salt, for example salts of ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, and the like. Non limiting examples of inorganic or metal salts include lithium, sodium, calcium, potassium, magnesium salts and the like.

Additionally, the salts of the compounds described herein, can exist in either hydrated or unhydrated (the anhydrous) form or as solvates with other solvent molecules. Non-limiting examples of hydrates include monohydrates, dihydrates, etc. Nonlimiting examples of solvates include ethanol solvates, acetone solvates, etc.

The term “solvates” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The compounds and salts described herein can exist in several tautomeric forms, including the enol and imine form, and the keto and enamine form and geometric isomers and mixtures thereof. Tautomers exist as mixtures of a tautomeric set in solution. In solid form, usually one tautomer predominates. Even though one tautomer may be described, the present application includes all tautomers of the present compounds. A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs.

Tautomerizations can be catalyzed by: Base: 1. deprotonation; 2. formation of a delocalized anion (e.g., an enolate); 3. protonation at a different position of the anion; Acid: 1. protonation; 2. formation of a delocalized cation; 3. deprotonation at a different position adjacent to the cation.

The terms below, as used herein, have the following meanings, unless indicated otherwise:
“Amino” refers to the —NH₂ radical.
“Cyano” refers to the —CN radical.
“Halo” or “halogen” refers to bromo, chloro, fluoro or iodo radical.
“Hydroxy” or “hydroxyl” refers to the —OH radical.
“Imino” refers to the =NH substituent.
“Nitro” refers to the —NO₂ radical.
“Oxo” refers to the ═O substituent.
“Thioxo” refers to the =S substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twelve carbon atoms, and having one or more carbon-carbon double bonds. Non-limiting examples of C₂-C₁₂ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twelve carbon atoms, and having one or more carbon-carbon triple bonds. Non-limiting examples of C₂-C₁₂ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain. Unless stated otherwise specifically in the specification, an alkynylene chain can be optionally substituted.

“Alkoxy” refers to a radical of the formula —ORₐ where Rₐ is an alkyl, alkenyl or alknyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₂-C₁₀ alkenyl, or C₂-C₁₀ alkynyl radical as defined above. Unless stated otherwise specifically in the specification, an alkyl carbonyl group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from phenyl (benzene), aceanthrylene, acenaphthylene, acephenanthrylene, anthracene, azulene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl” or “arylalkyl” refers to a radical of the formula —R_{b}—R_{c} where R_{b} is an alkylene group as defined above and R_{c} is one or more aryl radicals as defined above. Aralkyl radicals include, but are not limited to, benzyl, diphenylmethyl and the like. Unless stated otherwise specifically in the specification, an aralkyl group can be optionally substituted.

“Aralkenyl” or “arylalkenyl” refers to a radical of the formula —R_{b}—R_{c} where R_{b} is an alkenylene group as defined above and R_{c} is one or more aryl radicals as defined above. Unless stated otherwise specifically in the specification, an aralkenyl group can be optionally substituted.

“Aralkynyl” or “arylalkynyl” refers to a radical of the formula —R_{b}—Rₑ where R_{b} is an alkynylene group as defined above and R_{c} is one or more aryl radicals as defined above. Unless stated otherwise specifically in the specification, an aralkynyl group can be optionally substituted.

“Carbocyclyl,” “carbocyclic ring” or “carbocycle” refers to a ring structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused, bridged, or spiral ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused, bridged, or spiral ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused, bridged, or spiral ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl,” “heterocyclic ring” or “heterocycle” refers to a stable 3- to 20-membered non-aromatic, partially aromatic, or aromatic ring radical which consists of two to twelve carbon atoms and from one to six heteroatoms selected from the group consisting of nitrogen, oxygen and sulfur. Heterocyclycl or heterocyclic rings include heteroaryls as defined below. Unless stated otherwise specifically in the specification, the heterocyclyl radical can be a monocyclic, bicyclic, tricyclic or tetracyclic ring system, which can include fused, bridged, and spiral ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, aziridinyl, oextanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, pyridine-one, and the like. The point of attachment of the heterocyclyl, heterocyclic ring, or heterocycle to the rest of the molecule by a single bond is through a ring member atom, which can be carbon or nitrogen. Unless stated otherwise specifically in the specification, a heterocyclyl group can be optionally substituted.

“Heterocyclylalkyl” refers to a radical of the formula —R_{b}—Rₑ where R_{b} is an alkylene group as defined above and Rₑ is a heterocyclyl radical as defined above. Unless stated otherwise specifically in the specification, a heterocyclylalkyl group can be optionally substituted.

“Heterocyclylalkenyl” refers to a radical of the formula —R_{b}—Rₑ where R_{b} is an alkenylene group as defined above and Rₑ is a heterocyclyl radical as defined above. Unless stated otherwise specifically in the specification, a heterocyclylalkenyl group can be optionally substituted.

“Heterocyclylalkynyl” refers to a radical of the formula —R_{b}—Rₑ where R_{b} is an alkynylene group as defined above and Rₑ is a heterocyclyl radical as defined above. Unless stated otherwise specifically in the specification, a heterocyclylalkynyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical one to thirteen carbon atoms and one to six heteroatoms selected from the group consisting of nitrogen, oxygen and sulfur, as the ring member. For purposes of this invention, the heteroaryl radical can be a monocyclic, bicyclic, tricyclic or tetracyclic ring system, which can include fused or bridged ring systems, wherein at least one ring containing a heteroatom ring member is aromatic. The nitrogen, carbon or sulfur atoms in the heteroaryl radical can be optionally oxidized and the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, pyrazolopyridine, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Heteroarylalkenyl” refers to a radical of the formula —R_{b}—R_{f} where R_{b} is an alkenylene, chain as defined above and R_{f} is a heteroaryl radical as defined above. Unless stated otherwise specifically in the specification, a heteroarylalkenyl group can be optionally substituted.

“Heteroarylalkynyl” refers to a radical of the formula —R_{b}—R_{f} where R_{b} is an alkynylene chain as defined above and R_{f} is a heteroaryl radical as defined above. Unless stated otherwise specifically in the specification, a heteroarylalkynyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl, heteroarylalkyl, heteroarylalkenyl, heteroarylalkynyl, etc.) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom, such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, =NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol “

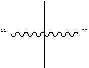

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example, “

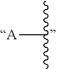

indicates that the chemical entity “A” is bonded to another chemical entity via the point of attachment bond. Furthermore, the specific point of attachment to the non-depicted chemical entity can be specified by inference. For example, the compound

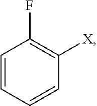

wherein X is “

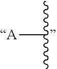

infers that the point of attachment bond is the bond by which X is depicted as being attached to the phenyl ring at the ortho position relative to fluorine.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The term “treating” is art-recognized and includes inhibiting a disease, disorder or condition in a subject, e.g., impeding its progress; and relieving the disease, disorder or condition, e.g., causing regression of the disease, disorder and/or condition. Treating the disease or condition includes ameliorating at least one symptom of the particular disease or condition, even if the underlying pathophysiology is not affected.

The term “preventing” is art-recognized and includes stopping a disease, disorder or condition from occurring in a subject, which may be predisposed to the disease, disorder and/or condition but has not yet been diagnosed as having it. Preventing a condition related to a disease includes stopping the condition from occurring after the disease has been diagnosed but before the condition has been diagnosed.

A “patient,” “subject,” or “host” to be treated by the subject method may mean either a human or non-human animal, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

The terms “prophylactic” or “therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic (i.e., it is intended to diminish, ameliorate, or stabilize the existing unwanted condition or side effects thereof).

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “ED50” is art-recognized. In certain embodiments, ED50 means the dose of a drug, which produces 50% of its maximum response or effect, or alternatively, the dose, which produces a pre-determined response in 50% of test subjects or preparations. The term “LD50” is art-recognized. In certain embodiments, LD50 means the dose of a drug, which is lethal in 50% of test subjects. The term “therapeutic index” is an art-recognized term, which refers to the therapeutic index of a drug, defined as LD50/ED50.

The terms “IC₅₀,” or “half maximal inhibitory concentration” is intended to refer to the concentration of a substance (e.g., a compound or a drug) that is required for 50% inhibition of a biological process, or component of a process, including a protein, subunit, organelle, ribonucleoprotein, etc.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

All percentages and ratios used herein, unless otherwise indicated, are by weight.

The term “neoplasm” refers to any abnormal mass of cells or tissue as a result of neoplasia. The neoplasm may be benign, potentially malignant (precancerous), or malignant (cancerous). An adenoma is an example of a neoplasm.

The terms “adenoma”, “colon adenoma” and “polyp” are used herein to describe any precancerous neoplasm of the colon.

The term “colon” as used herein is intended to encompass the right colon (including the cecum), the transverse colon, the left colon and the rectum.

The terms “colorectal cancer” and “colon cancer” are used interchangeably herein to refer to any cancerous neoplasia of the colon (including the rectum, as defined above).

The terms “gene expression” or “protein expression” includes any information pertaining to the amount of gene transcript or protein present in a sample, as well as information about the rate at which genes or proteins are produced or are accumulating or being degraded (e.g., reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The terms “healthy” and “normal” are used interchangeably herein to refer to a subject or particular cell or tissue that is devoid (at least to the limit of detection) of a disease condition.

The term “nucleic acid” refers to polynucleotides such as deoxyribonucleic acid (DNA), and, where appropriate, ribonucleic acid (RNA). The term should also be understood to include analogues of either RNA or DNA made from nucleotide analogues, and, as applicable to the embodiment being described, single-stranded (such as sense or antisense) and double-stranded polynucleotides. In some embodiments, “nucleic acid” refers to inhibitory nucleic acids. Some categories of inhibitory nucleic acid compounds include antisense nucleic acids, RNAi constructs, and catalytic nucleic acid constructs. Such categories of nucleic acids are well-known in the art.

Embodiments described herein relate to compounds and methods of modulating SCD activity (e.g., 15-PGDH activity), modulating tissue prostaglandin levels, and/or treating diseases, disorders, or conditions in which it is desired to modulate 15-PGDH activity and/or prostaglandin levels.

“Inhibitors,” “activators,” and “modulators” of 15-PGDH expression or of 15-PGDH activity are used to refer to inhibitory, activating, or modulating molecules, respectively, identified using in vitro and in vivo assays for 15-PGDH expression or 15-PGDH activity, e.g., ligands, agonists, antagonists, and their homologs and mimetics. The term “modulator” includes inhibitors and activators. Inhibitors are agents that, e.g., inhibit expression of 15-PGDH or bind to, partially or totally block stimulation, decrease, prevent, delay activation, inactivate, desensitize, or down regulate the activity of 15-PGDH, e.g., antagonists. Activators are agents that, e.g., induce or activate the expression of a 15-PGDH or bind to, stimulate, stabilize, increase, open, activate, facilitate, or enhance activation, sensitize or up regulate the activity of 15-PGDH, e.g., agonists. Modulators include naturally occurring and synthetic ligands, small chemical molecules, and the like.

15-PGDH inhibitors described herein can provide a pharmacologic method for elevating prostaglandin levels in tissue. Known activities of prostaglandins include promoting hair growth, promoting skin pigmentation, and promoting skin darkening or the appearance of skin tanning. Known activities of prostaglandins also include ameliorating pulmonary artery hypertension. 15-PGDH inhibitors described herein may also be utilized to increase tissue stem cell numbers for purposes that would include increasing resistance to tissue damage by radiation, increasing resistance to environmental exposures to radiation, increasing stem cell numbers to increase fitness of bone marrow or other types of transplantation (through either in vivo exposure to 15-PGDH inhibitors described herein to increase stem cell numbers prior to harvest of a transplanted tissue, or through ex vivo exposure of a harvested tissue prior to transplant into a recipient host, or through treatment of the graft recipient). 15-PGDH inhibitors described herein may also be utilized for purposes that would include promoting liver regeneration, including liver regeneration after liver resection, and liver regeneration after toxic insults, which for example may be the toxic insult of acetaminophen overdose. Prostaglandin signaling is also known to promote wound healing, protect the stomach from ulceration, and promote healing of ulcers of stomach and intestines. Additionally, 15-PGDH inhibitors described herein can promote activity of human keratinocytes in “healing” scratches across cultures of keratinocyte cells. Hence, 15-PGDH inhibitors described herein may be utilized to also heal ulcers of other tissues, including, but not limited to skin, and including but not limited to diabetic ulcers. Further, 15-PGDH inhibitors described herein may be utilized for the treatment of erectile dysfunction.

15-PGDH inhibitors described herein can be identified using assays in which putative modulator compounds are applied to cells expressing 15-PGDH and then the functional effects on 15-PGDH activity are determined. Samples or assays comprising 15-PGDH that are treated with a potential activator, inhibitor, or modulator are compared to control samples without the inhibitor, activator, or modulator to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%.

Agents tested as modulators of SCD (e.g., 15-PGDH) can be any small chemical molecule or compound. Typically, test compounds will be small chemical molecules, natural products, or peptides. The assays are designed to screen large chemical libraries by automating the assay steps and providing compounds from any convenient source to assays, which are typically run in parallel (e.g., in microtiter formats on microtiter plates in robotic assays). Modulators also include agents designed to increase the level of 15-PGDH mRNA or the level of translation from an mRNA.

In some embodiments, the modulator of SCD can be an SCD inhibitor that can be administered to tissue or blood of a subject at an amount effective to inhibit the activity of a short chain dehydrogenase enzyme. The SCD inhibitor can be a 15-PGDH inhibitor that can be administered to tissue or blood of a subject at an amount effective to increase prostaglandin levels in the tissue or blood. The 15-PGDH inhibitor can include a compound having a structure of formula (IA):

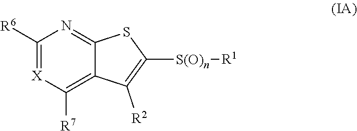

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
R¹⁰ is —OH, halogen, alkyl, or alkoxy;
X is N or CH;
m is 0, 1, or 2;
n is 0, 1, or 2; and
t is 0, 1, or 2.

In some embodiment, the 15-PGDH inhibitor can include a compound having a structure of formula (I):

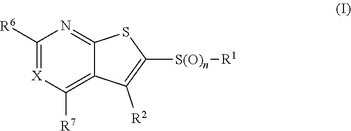

or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclycl or heteroaryl, each of which is optionally substituted with one or more R³; R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R′)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
X is N or CH;
m is 0, 1, or 2;
n is 0, 1, or 2; and
t is 0, 1, or 2.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In still other embodiments, R² is —NH₂.

In some embodiments, R⁶ is 5- to 6-membered heterocyclycl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₆ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₆ haloalkyl, 3- to 6-membered cycloalkyl, phenyl, 5- to 10-membered heteroaryl each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In other embodiments, the 15-PGDH inhibitor can include a compound selected from the group consisting of

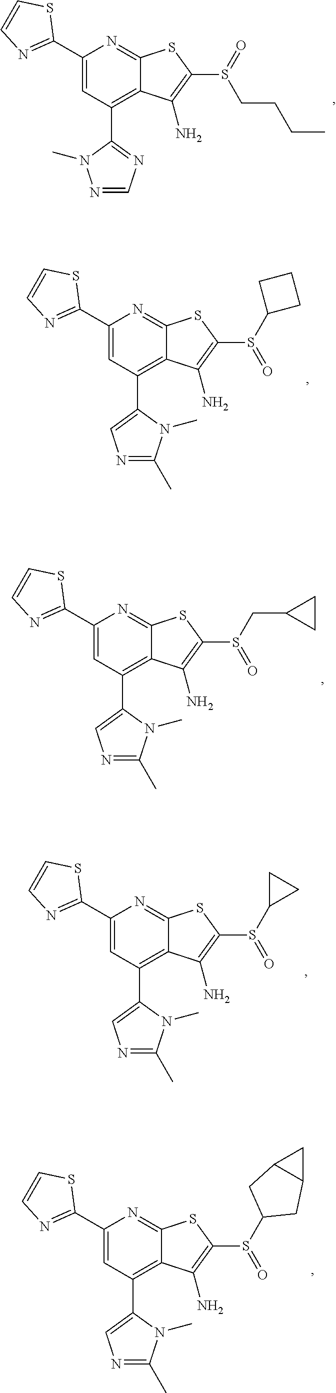 [SMILES: CCCCS(=O)c1sc2nc(-c3nccs3)cc(-c3ncnn3C)c2c1N ; Cc1ncc(-c2cc(-c3nccs3)nc3sc(S(=O)C4CCC4)c(N)c23)n1C ; Cc1ncc(-c2cc(-c3nccs3)nc3sc(S(=O)CC4CC4)c(N)c23)n1C ; Cc1ncc(-c2cc(-c3nccs3)nc3sc(S(=O)C4CC4)c(N)c23)n1C ; Cc1ncc(-c2cc(-c3nccs3)nc3sc(S(=O)C4CC5CC5C4)c(N)c23)n1C]

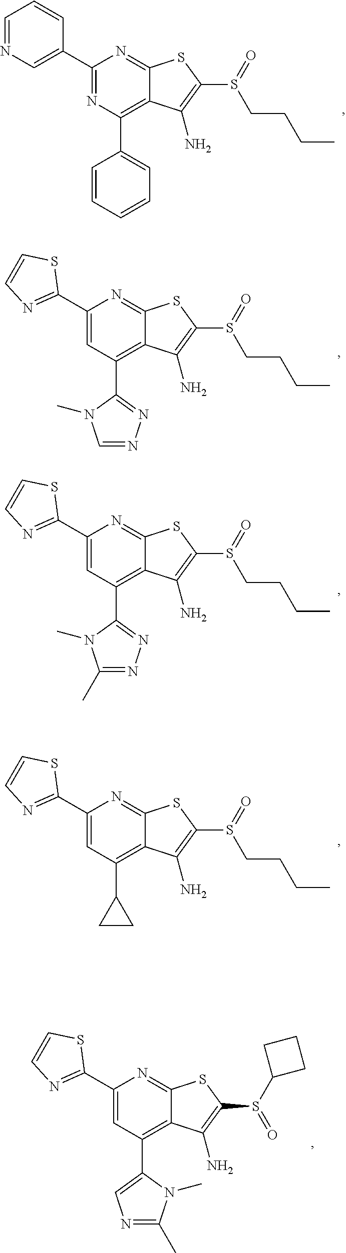 [SMILES: CCCCS(=O)c1sc2nc(-c3cccnc3)nc(-c3ccccc3)c2c1N ; CCCCS(=O)c1sc2nc(-c3nccs3)cc(-c3nncn3C)c2c1N ; CCCCS(=O)c1sc2nc(-c3nccs3)cc(-c3nnc(C)n3C)c2c1N ; CCCCS(=O)c1sc2nc(-c3nccs3)cc(C3CC3)c2c1N ; Cc1ncc(-c2cc(-c3nccs3)nc3sc([S@](=O)C4CCC4)c(N)c23)n1C]

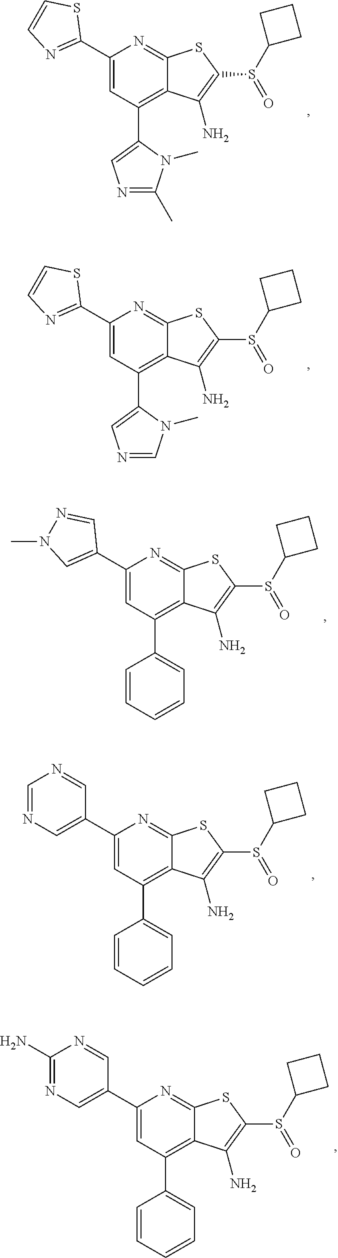 [SMILES: Cc1ncc(-c2cc(-c3nccs3)nc3sc([S@@](=O)C4CCC4)c(N)c23)n1C ; Cn1cncc1-c1cc(-c2nccs2)nc2sc(S(=O)C3CCC3)c(N)c12 ; Cn1cc(-c2cc(-c3ccccc3)c3c(N)c(S(=O)C4CCC4)sc3n2)cn1 ; Nc1c(S(=O)C2CCC2)sc2nc(-c3cncnc3)cc(-c3ccccc3)c12 ; Nc1ncc(-c2cc(-c3ccccc3)c3c(N)c(S(=O)C4CCC4)sc3n2)cn1]

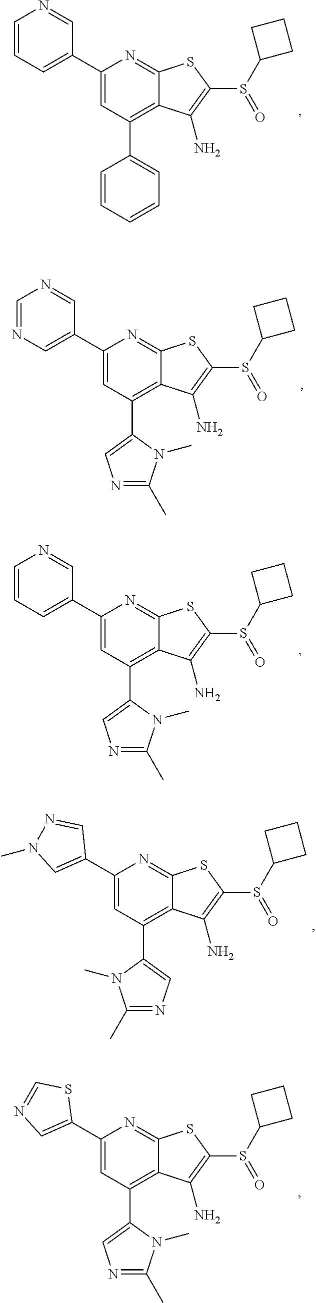 [SMILES: Nc1c(S(=O)C2CCC2)sc2nc(-c3cccnc3)cc(-c3ccccc3)c12 ; Cc1ncc(-c2cc(-c3cncnc3)nc3sc(S(=O)C4CCC4)c(N)c23)n1C ; Cc1ncc(-c2cc(-c3cccnc3)nc3sc(S(=O)C4CCC4)c(N)c23)n1C ; Cc1ncc(-c2cc(-c3cnn(C)c3)nc3sc(S(=O)C4CCC4)c(N)c23)n1C ; Cc1ncc(-c2cc(-c3cncs3)nc3sc(S(=O)C4CCC4)c(N)c23)n1C]

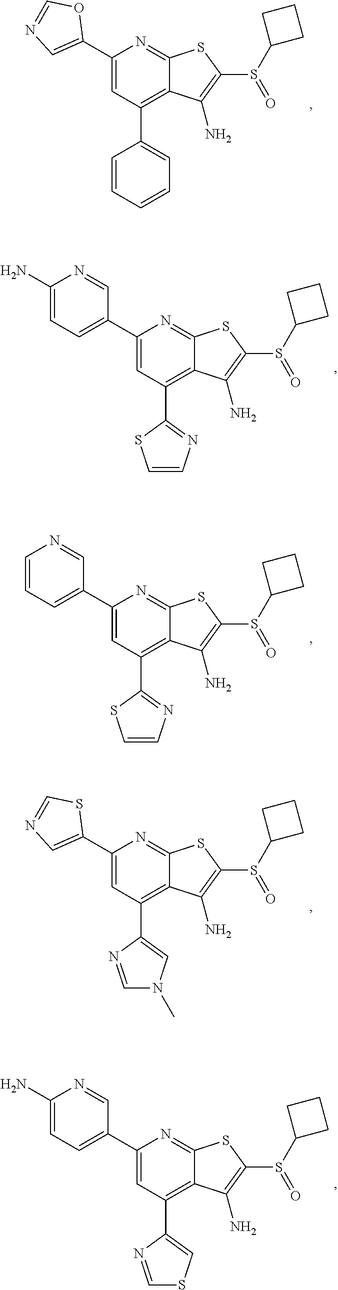 [SMILES: Nc1c(S(=O)C2CCC2)sc2nc(-c3cnco3)cc(-c3ccccc3)c12 ; Nc1ccc(-c2cc(-c3nccs3)c3c(N)c(S(=O)C4CCC4)sc3n2)cn1 ; Nc1c(S(=O)C2CCC2)sc2nc(-c3cccnc3)cc(-c3nccs3)c12 ; Cn1cnc(-c2cc(-c3cncs3)nc3sc(S(=O)C4CCC4)c(N)c23)c1 ; Nc1ccc(-c2cc(-c3cscn3)c3c(N)c(S(=O)C4CCC4)sc3n2)cn1]

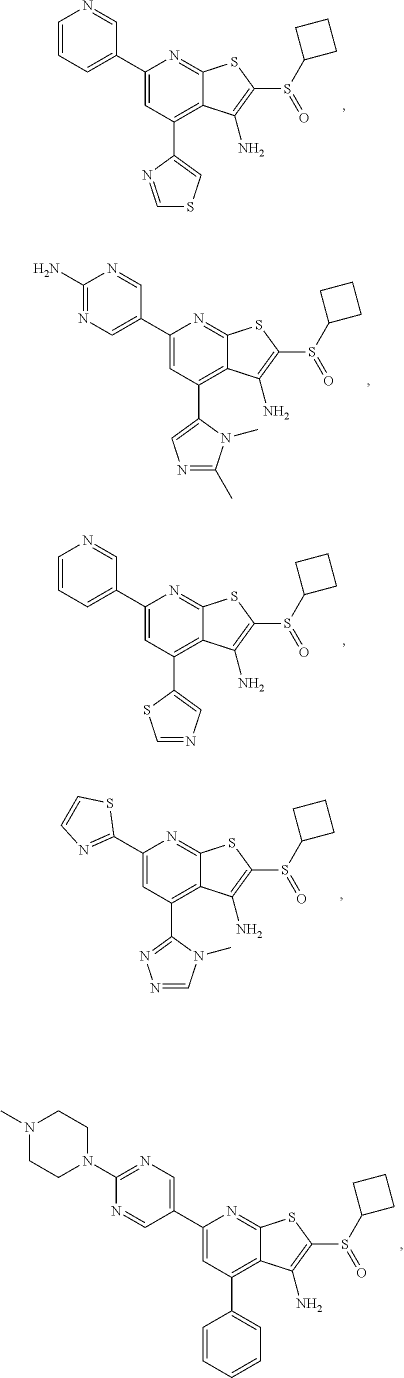 [SMILES: Nc1c(S(=O)C2CCC2)sc2nc(-c3cccnc3)cc(-c3cscn3)c12 ; Cc1ncc(-c2cc(-c3cnc(N)nc3)nc3sc(S(=O)C4CCC4)c(N)c23)n1C ; Nc1c(S(=O)C2CCC2)sc2nc(-c3cccnc3)cc(-c3cncs3)c12 ; Cn1cnnc1-c1cc(-c2nccs2)nc2sc(S(=O)C3CCC3)c(N)c12 ; CN1CCN(c2ncc(-c3cc(-c4ccccc4)c4c(N)c(S(=O)C5CCC5)sc4n3)cn2)CC1]

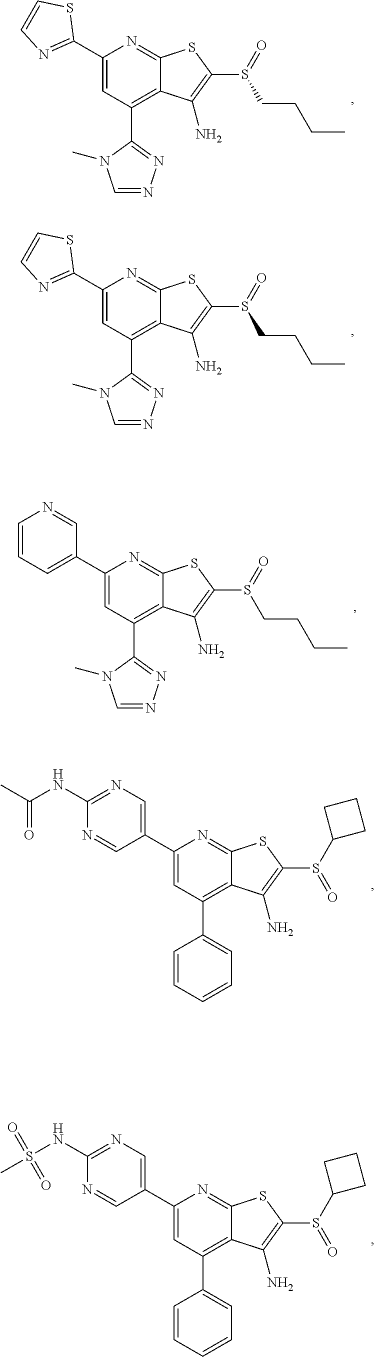

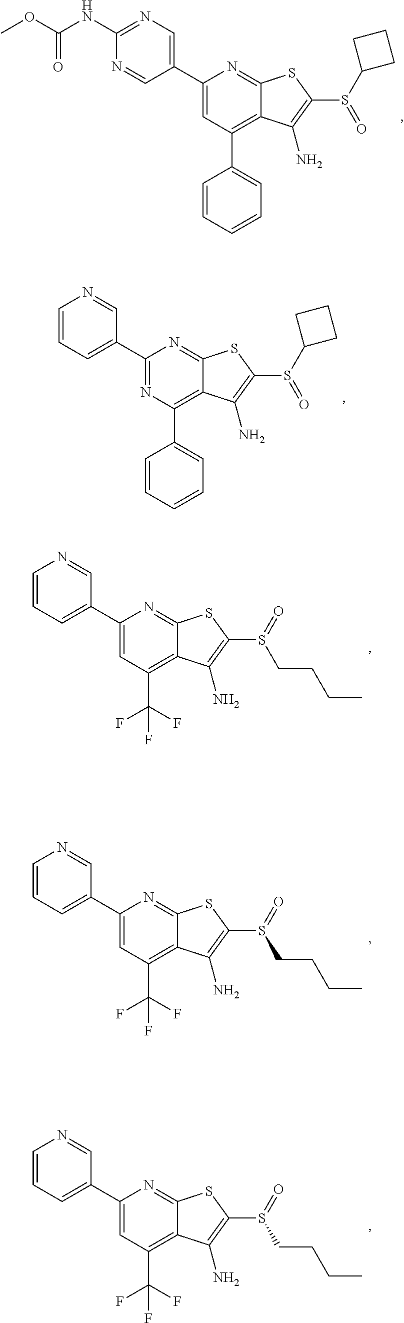 [SMILES: COC(=O)Nc1ncc(-c2cc(-c3ccccc3)c3c(N)c(S(=O)C4CCC4)sc3n2)cn1 ; Nc1c(S(=O)C2CCC2)sc2nc(-c3cccnc3)nc(-c3ccccc3)c12 ; CCCCS(=O)c1sc2nc(-c3cccnc3)cc(C(F)(F)F)c2c1N ; CCCC[S@](=O)c1sc2nc(-c3cccnc3)cc(C(F)(F)F)c2c1N ; CCCC[S@@](=O)c1sc2nc(-c3cccnc3)cc(C(F)(F)F)c2c1N]

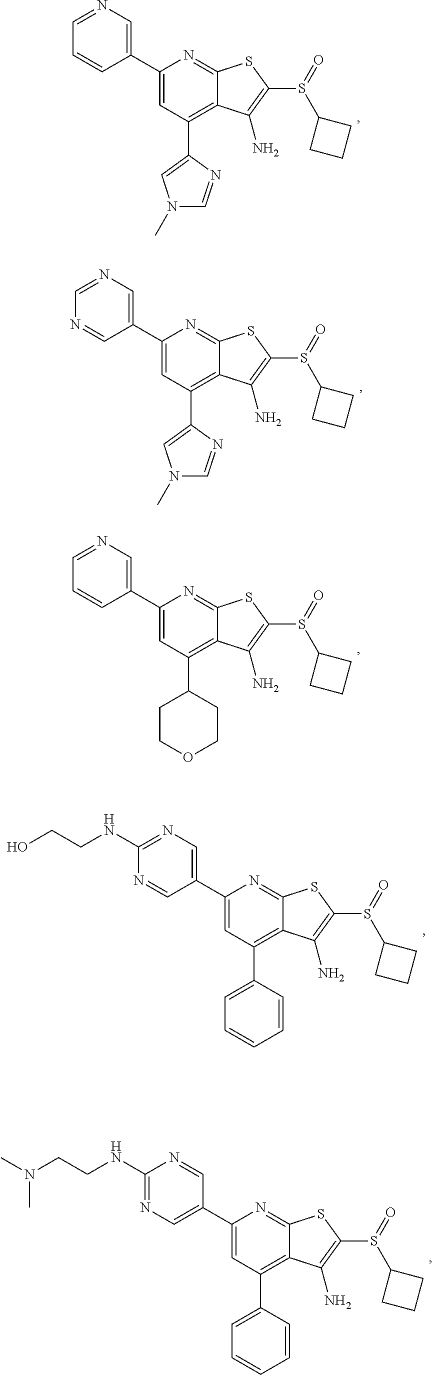 [SMILES: Cn1cnc(-c2cc(-c3cccnc3)nc3sc(S(=O)C4CCC4)c(N)c23)c1 ; Cn1cnc(-c2cc(-c3cncnc3)nc3sc(S(=O)C4CCC4)c(N)c23)c1 ; Nc1c(S(=O)C2CCC2)sc2nc(-c3cccnc3)cc(C3CCOCC3)c12 ; Nc1c(S(=O)C2CCC2)sc2nc(-c3cnc(NCCO)nc3)cc(-c3ccccc3)c12 ; CN(C)CCNc1ncc(-c2cc(-c3ccccc3)c3c(N)c(S(=O)C4CCC4)sc3n2)cn1]

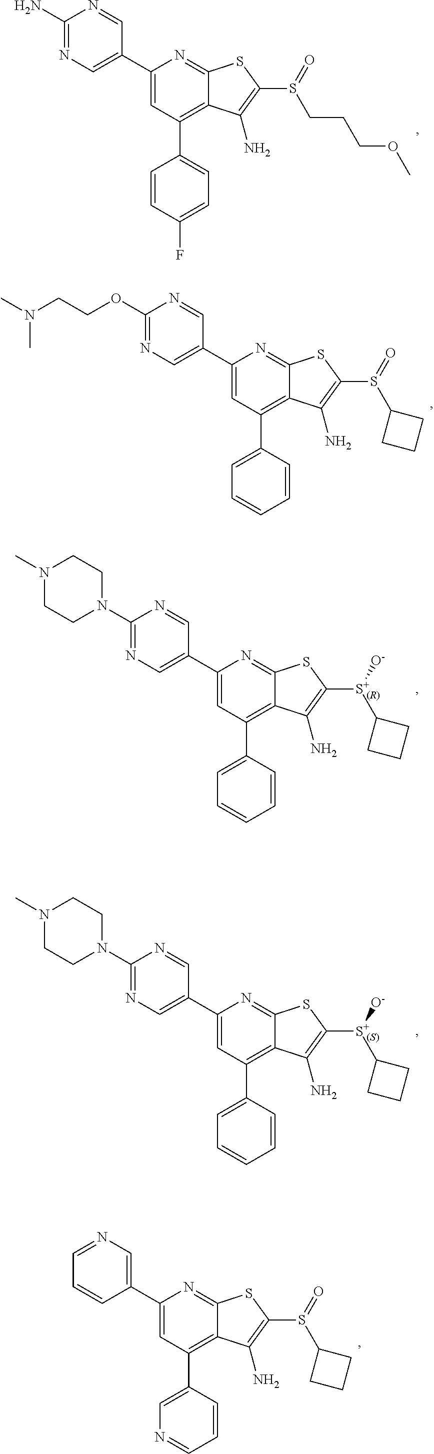

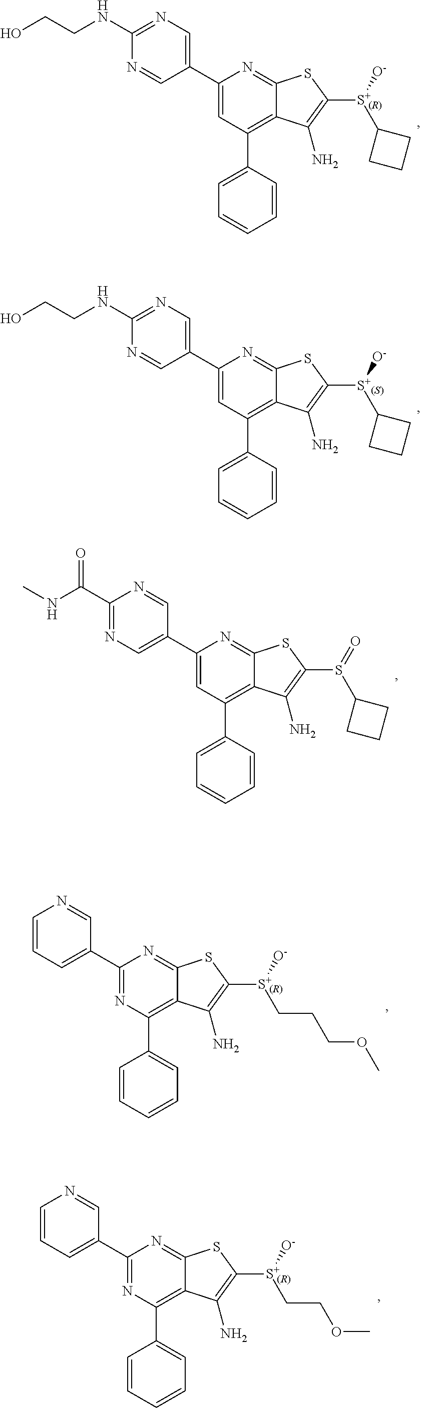

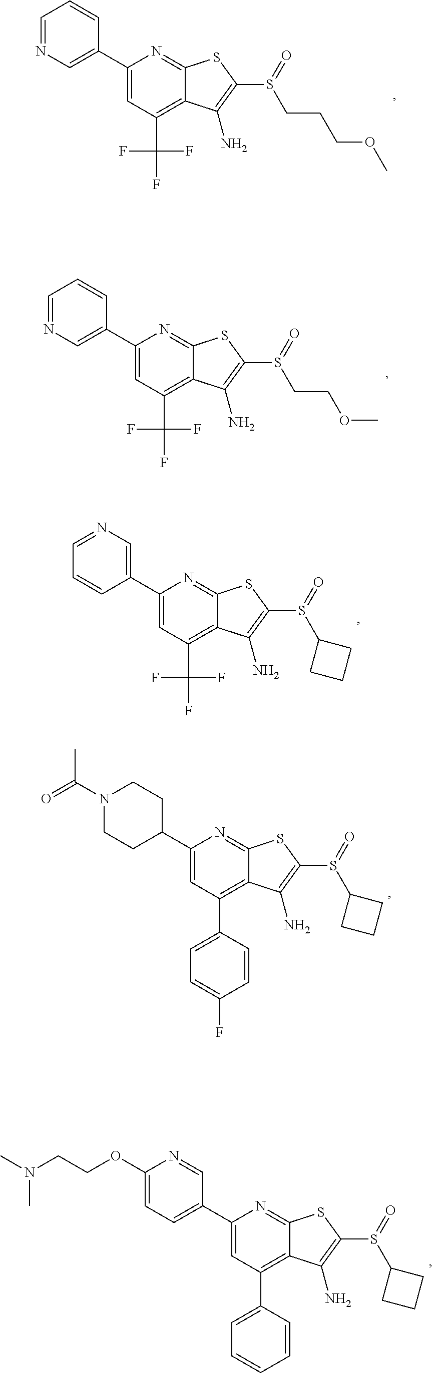

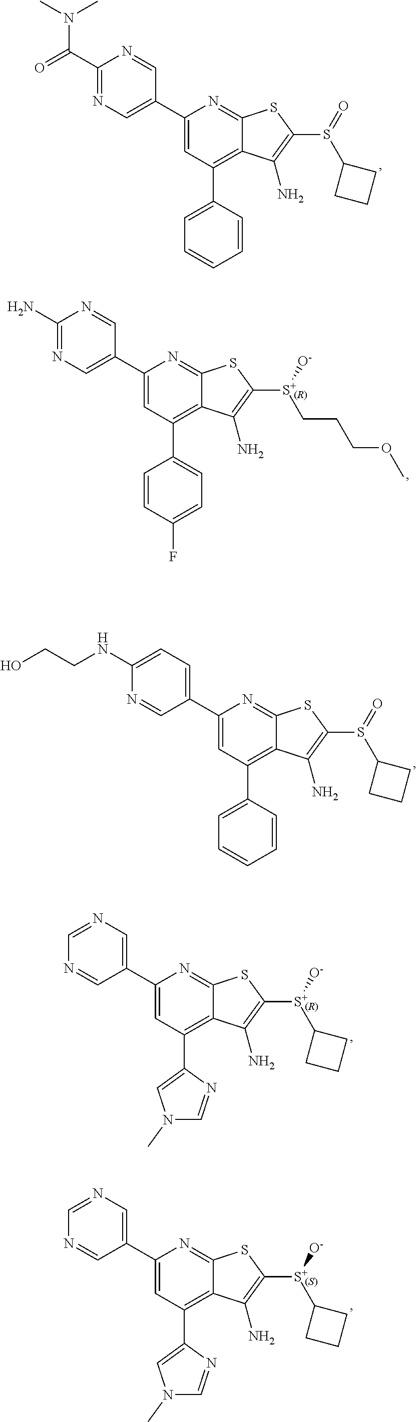

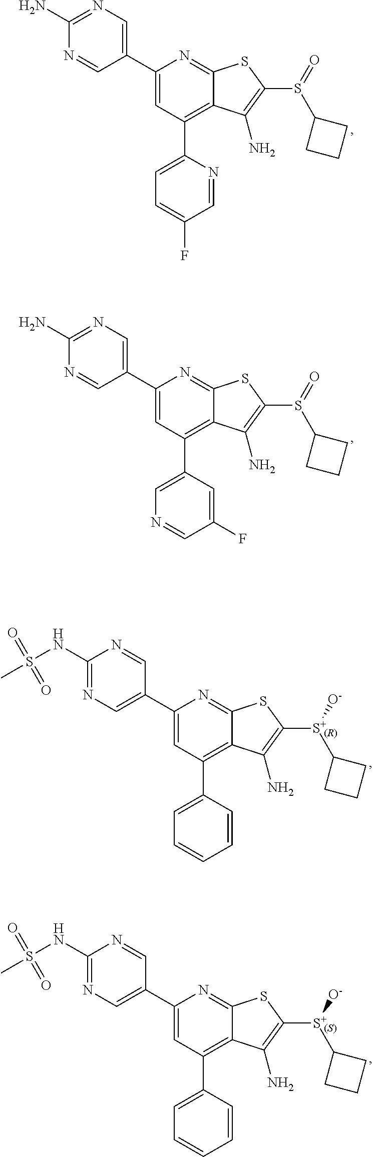

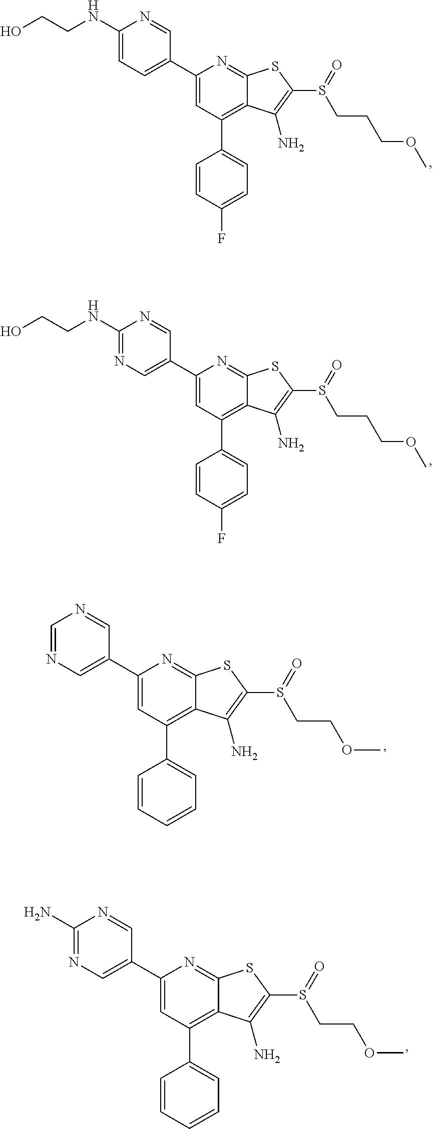 [SMILES: COCCCS(=O)c1sc2nc(-c3ccc(NCCO)nc3)cc(-c3ccc(F)cc3)c2c1N ; COCCCS(=O)c1sc2nc(-c3cnc(NCCO)nc3)cc(-c3ccc(F)cc3)c2c1N ; COCCS(=O)c1sc2nc(-c3cncnc3)cc(-c3ccccc3)c2c1N ; COCCS(=O)c1sc2nc(-c3cnc(N)nc3)cc(-c3ccccc3)c2c1N]

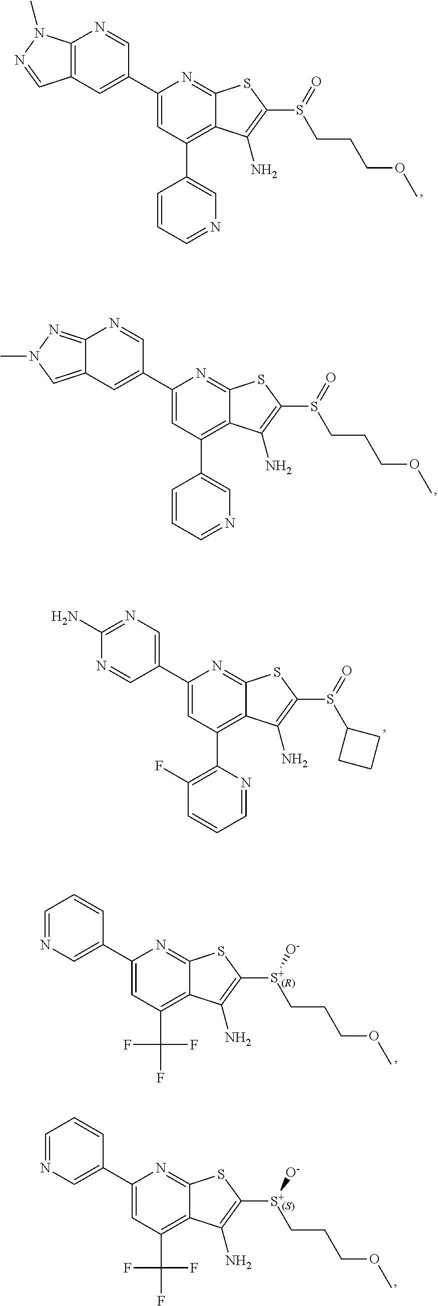

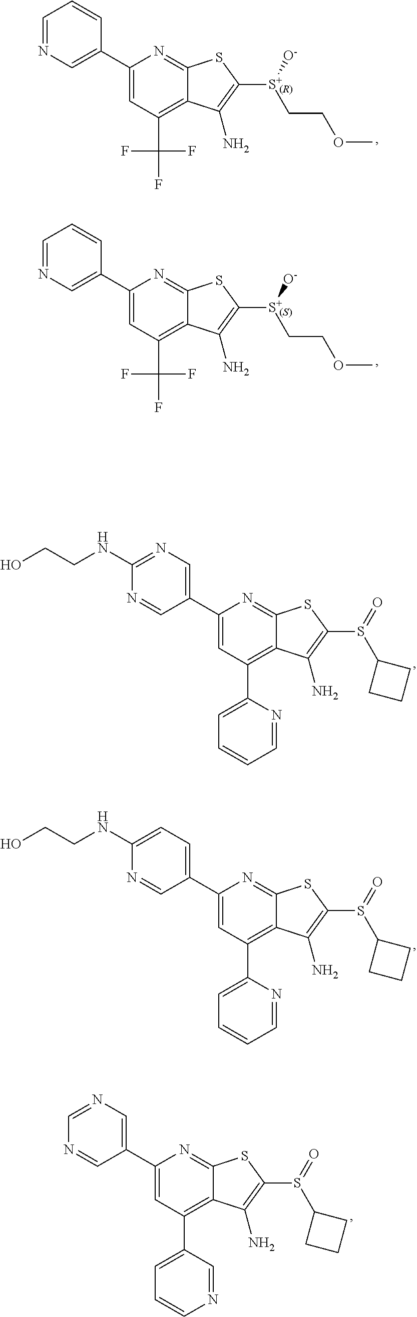

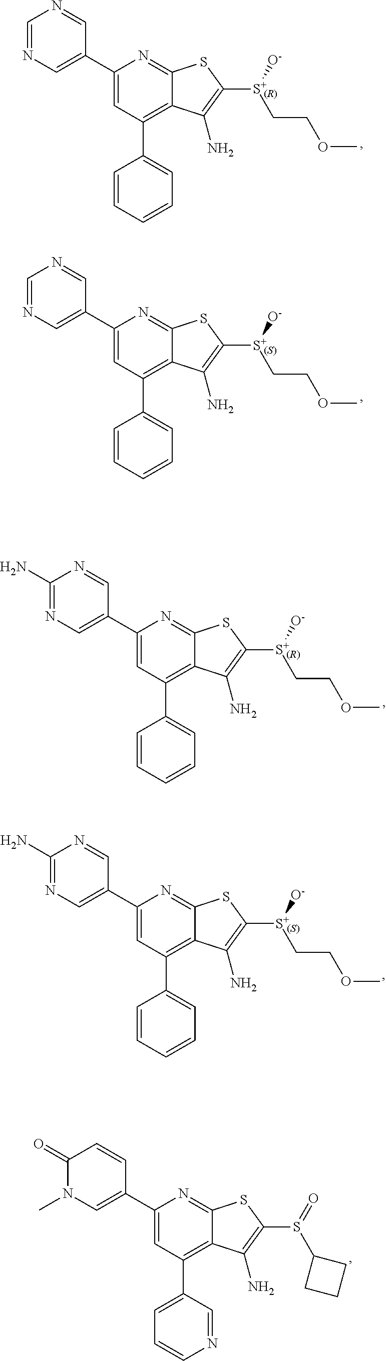

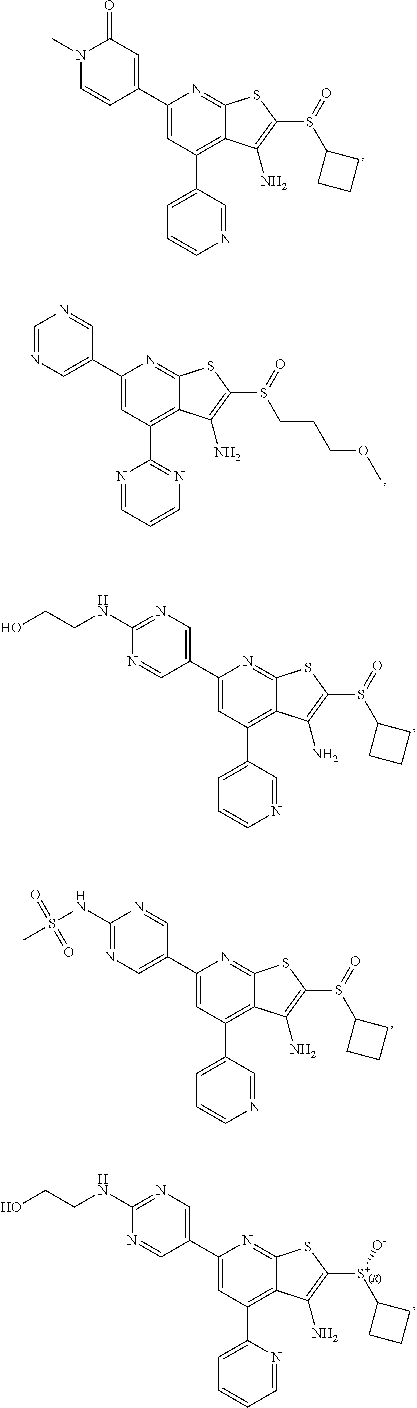

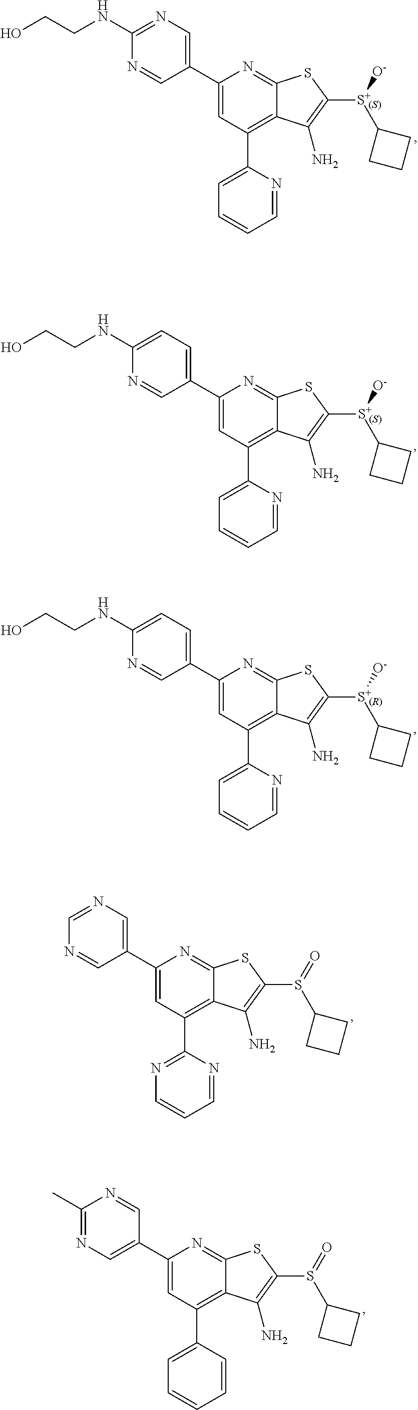

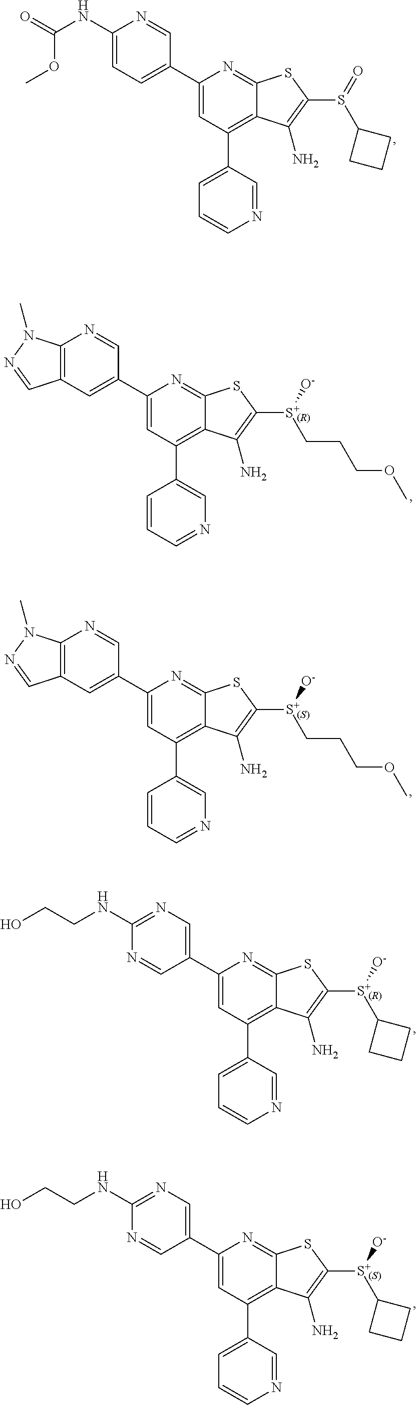

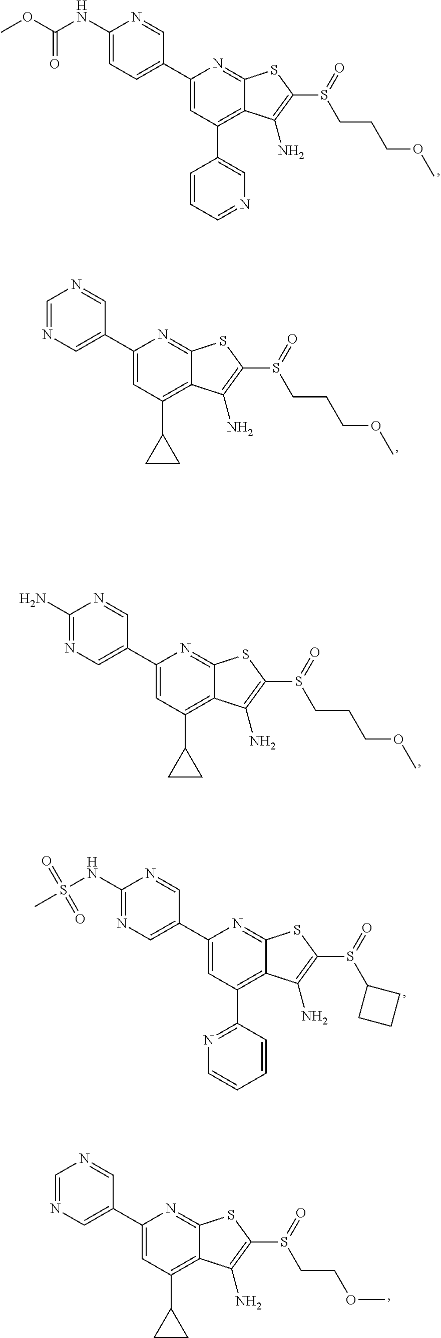

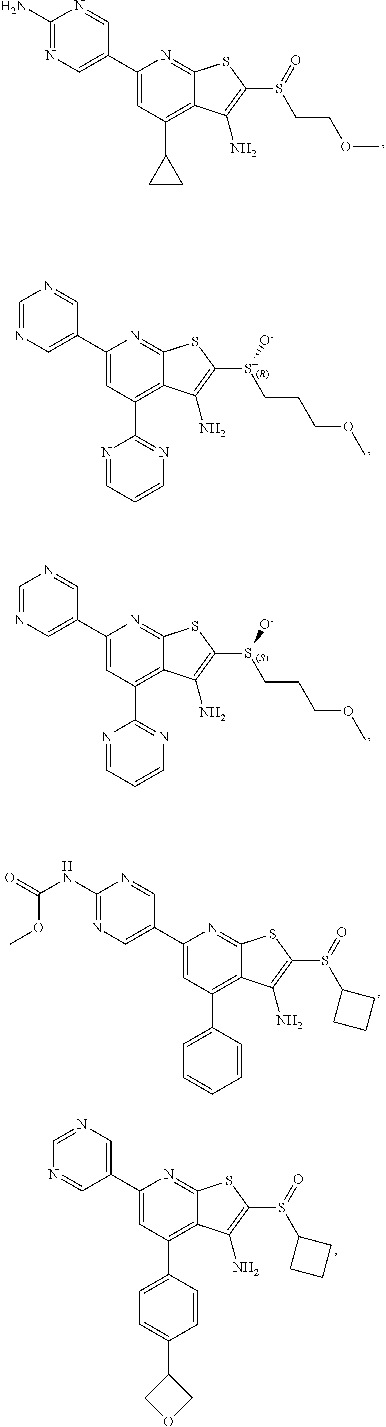

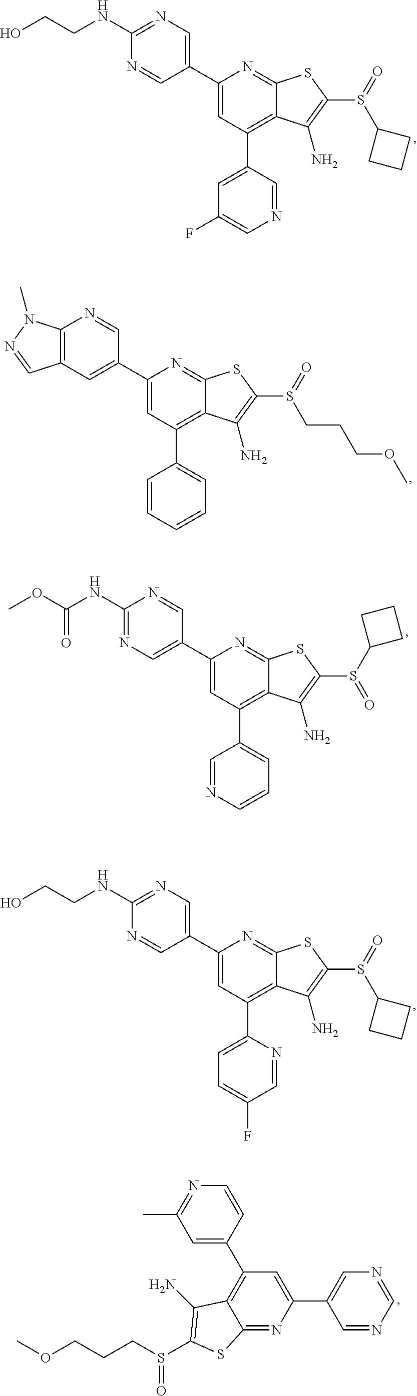

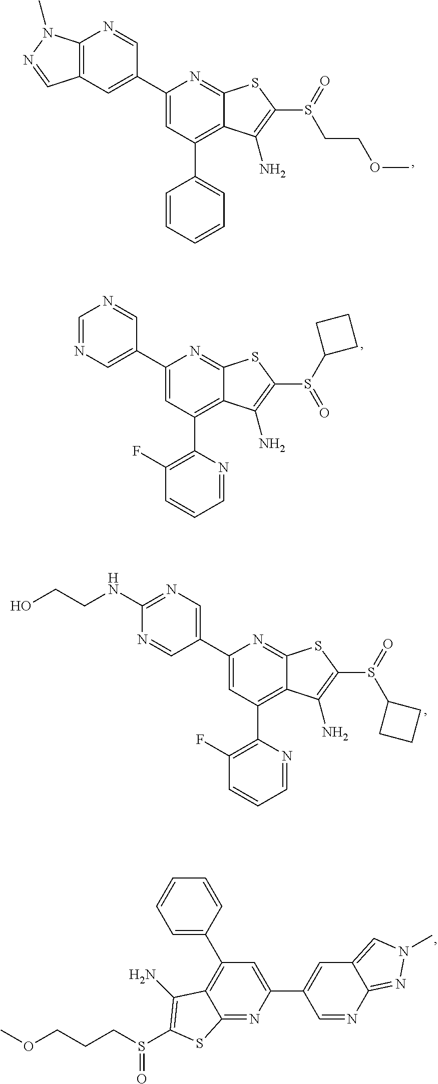

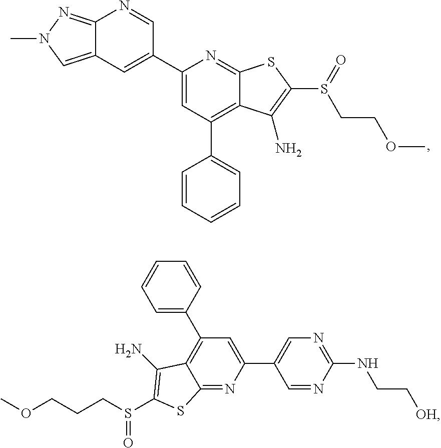

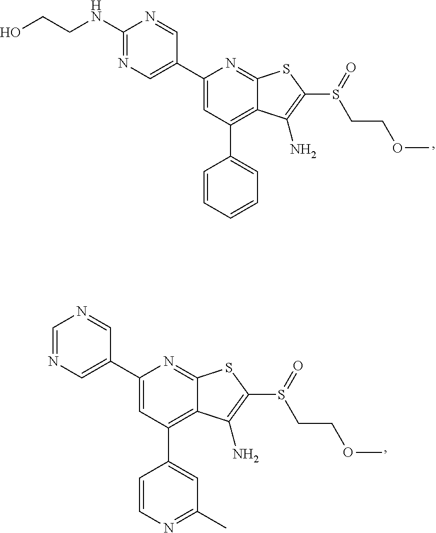 [SMILES: COCCS(=O)c1sc2nc(-c3cnc(NCCO)nc3)cc(-c3ccccc3)c2c1N ; COCCS(=O)c1sc2nc(-c3cncnc3)cc(-c3ccnc(C)c3)c2c1N]

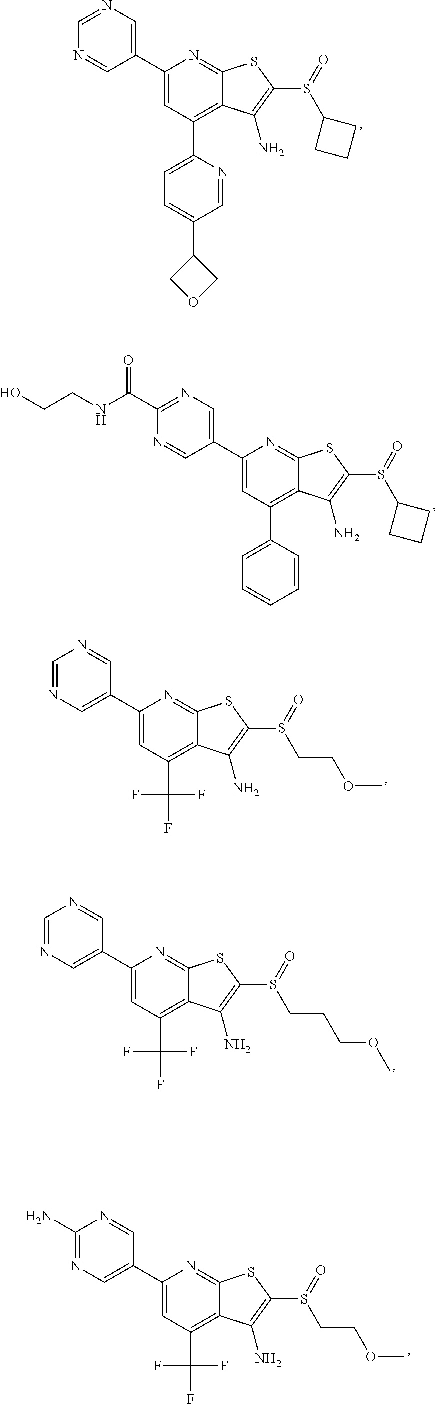 [SMILES: Nc1c(S(=O)C2CCC2)sc2nc(-c3cncnc3)cc(-c3ccc(C4COC4)cn3)c12 ; Nc1c(S(=O)C2CCC2)sc2nc(-c3cnc(C(=O)NCCO)nc3)cc(-c3ccccc3)c12 ; COCCS(=O)c1sc2nc(-c3cncnc3)cc(C(F)(F)F)c2c1N ; COCCCS(=O)c1sc2nc(-c3cncnc3)cc(C(F)(F)F)c2c1N ; COCCS(=O)c1sc2nc(-c3cnc(N)nc3)cc(C(F)(F)F)c2c1N]

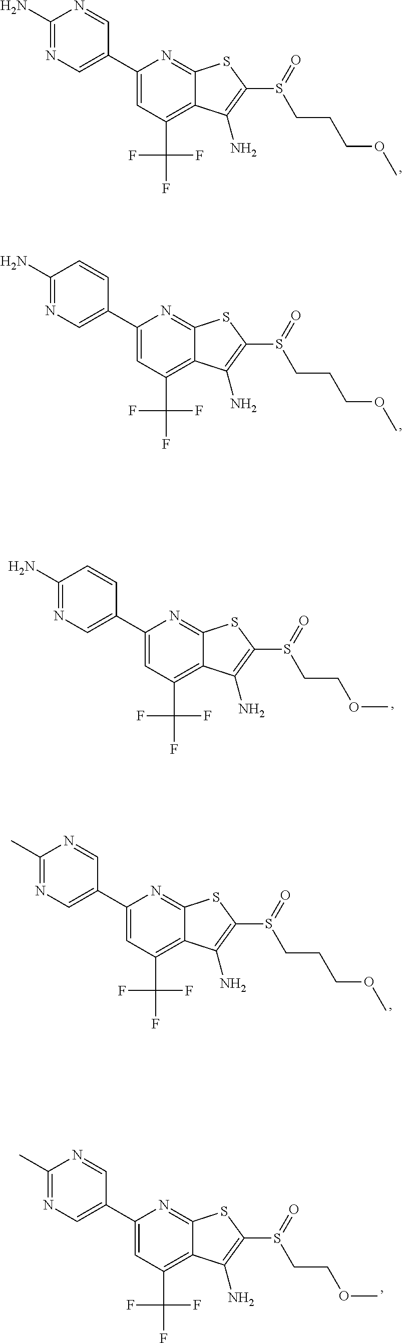

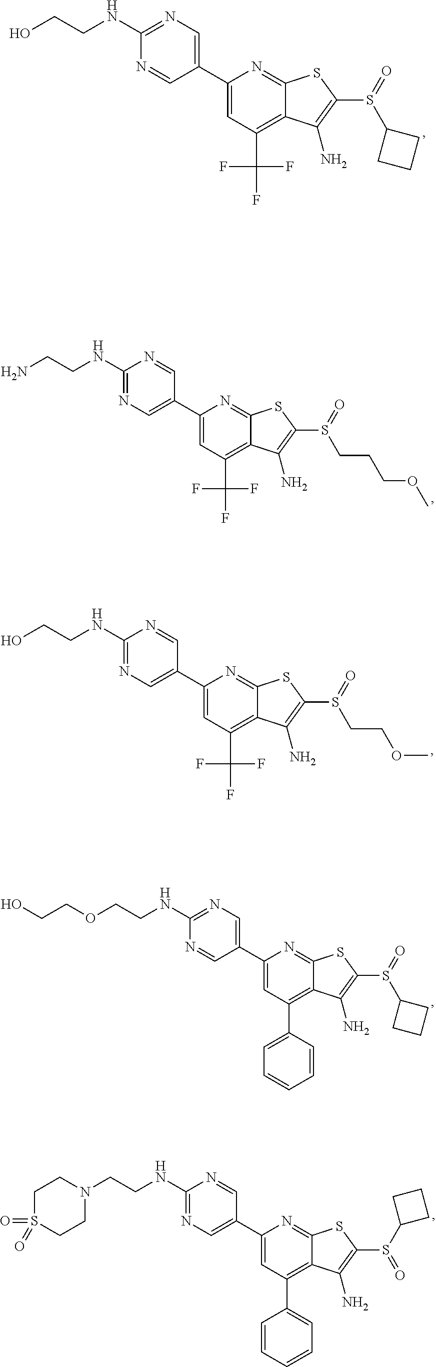 [SMILES: Nc1c(S(=O)C2CCC2)sc2nc(-c3cnc(NCCO)nc3)cc(C(F)(F)F)c12 ; COCCCS(=O)c1sc2nc(-c3cnc(NCCN)nc3)cc(C(F)(F)F)c2c1N ; COCCS(=O)c1sc2nc(-c3cnc(NCCO)nc3)cc(C(F)(F)F)c2c1N ; Nc1c(S(=O)C2CCC2)sc2nc(-c3cnc(NCCOCCO)nc3)cc(-c3ccccc3)c12 ; Nc1c(S(=O)C2CCC2)sc2nc(-c3cnc(NCCN4CCS(=O)(=O)CC4)nc3)cc(-c3ccccc3)c12]

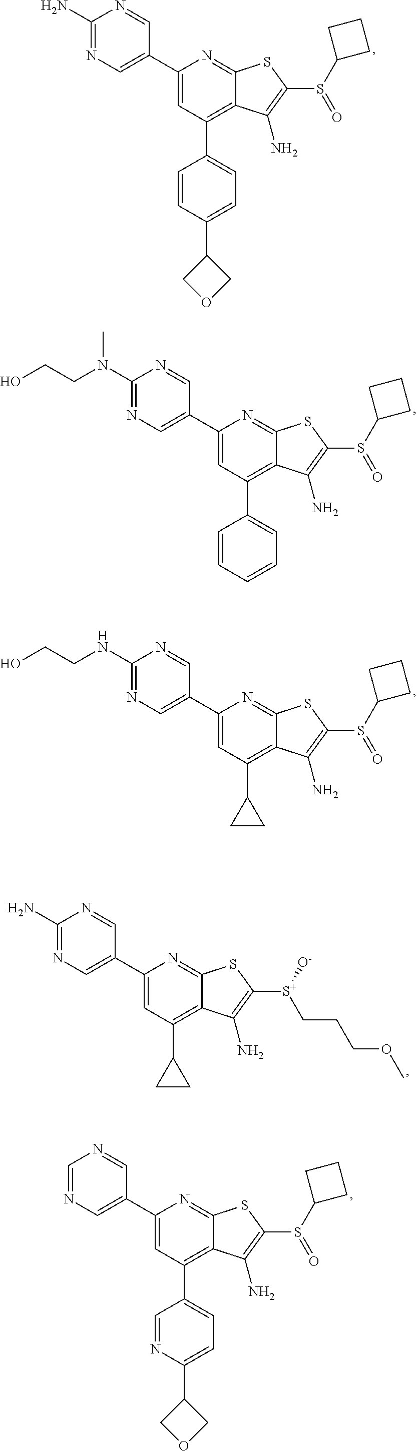 [SMILES: Nc1ncc(-c2cc(-c3ccc(C4COC4)cc3)c3c(N)c(S(=O)C4CCC4)sc3n2)cn1 ; CN(CCO)c1ncc(-c2cc(-c3ccccc3)c3c(N)c(S(=O)C4CCC4)sc3n2)cn1 ; Nc1c(S(=O)C2CCC2)sc2nc(-c3cnc(NCCO)nc3)cc(C3CC3)c12 ; COCCC[S@@+]([O-])c1sc2nc(-c3cnc(N)nc3)cc(C3CC3)c2c1N ; Nc1c(S(=O)C2CCC2)sc2nc(-c3cncnc3)cc(-c3ccc(C4COC4)nc3)c12]

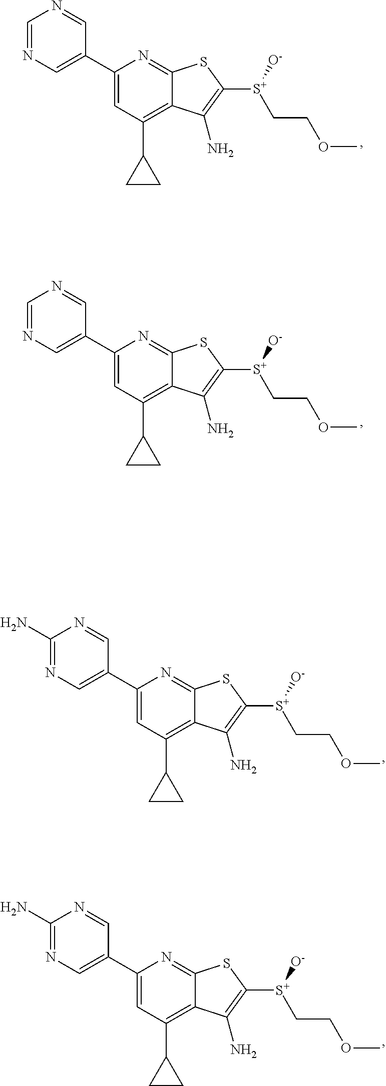 [SMILES: COCC[S@@+]([O-])c1sc2nc(-c3cncnc3)cc(C3CC3)c2c1N ; COCC[S@+]([O-])c1sc2nc(-c3cncnc3)cc(C3CC3)c2c1N ; COCC[S@@+]([O-])c1sc2nc(-c3cnc(N)nc3)cc(C3CC3)c2c1N ; COCC[S@+]([O-])c1sc2nc(-c3cnc(N)nc3)cc(C3CC3)c2c1N]

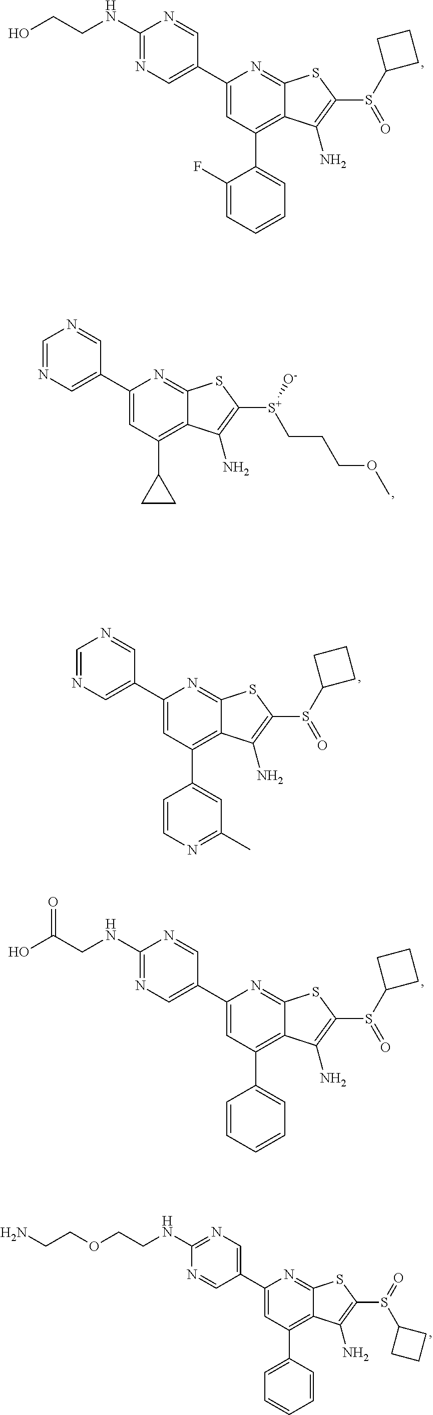 [SMILES: Nc1c(S(=O)C2CCC2)sc2nc(-c3cnc(NCCO)nc3)cc(-c3ccccc3F)c12 ; COCCC[S@@+]([O-])c1sc2nc(-c3cncnc3)cc(C3CC3)c2c1N ; Cc1cc(-c2cc(-c3cncnc3)nc3sc(S(=O)C4CCC4)c(N)c23)ccn1 ; Nc1c(S(=O)C2CCC2)sc2nc(-c3cnc(NCC(=O)O)nc3)cc(-c3ccccc3)c12 ; NCCOCCNc1ncc(-c2cc(-c3ccccc3)c3c(N)c(S(=O)C4CCC4)sc3n2)cn1]

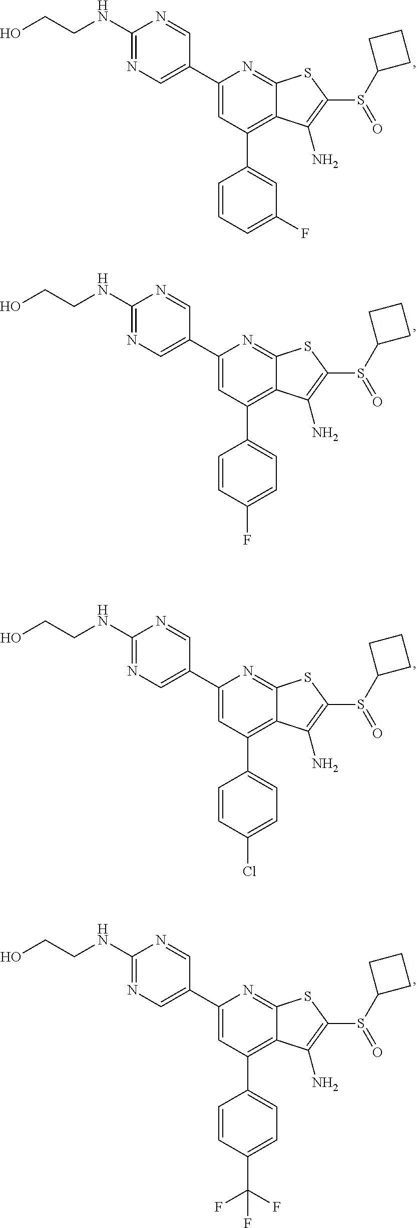 [SMILES: Nc1c(S(=O)C2CCC2)sc2nc(-c3cnc(NCCO)nc3)cc(-c3cccc(F)c3)c12 ; Nc1c(S(=O)C2CCC2)sc2nc(-c3cnc(NCCO)nc3)cc(-c3ccc(F)cc3)c12 ; Nc1c(S(=O)C2CCC2)sc2nc(-c3cnc(NCCO)nc3)cc(-c3ccc(Cl)cc3)c12 ; Nc1c(S(=O)C2CCC2)sc2nc(-c3cnc(NCCO)nc3)cc(-c3ccc(C(F)(F)F)cc3)c12]

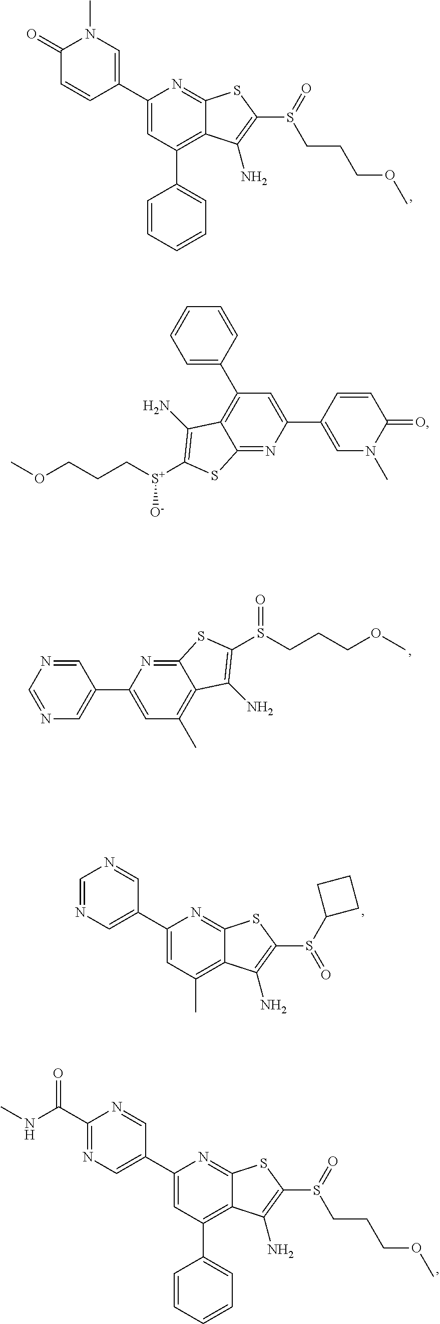

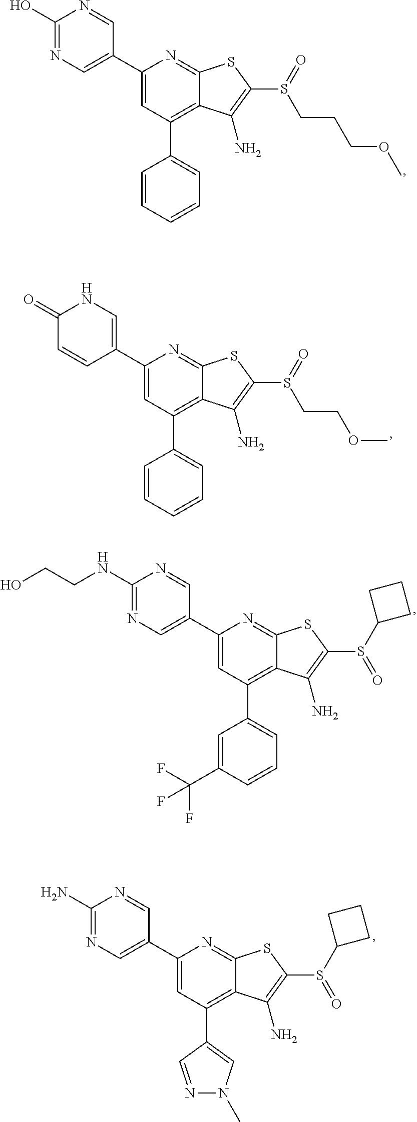 [SMILES: COCCCS(=O)c1sc2nc(-c3cnc(O)nc3)cc(-c3ccccc3)c2c1N ; COCCS(=O)c1sc2nc(-c3ccc(=O)[nH]c3)cc(-c3ccccc3)c2c1N ; Nc1c(S(=O)C2CCC2)sc2nc(-c3cnc(NCCO)nc3)cc(-c3cccc(C(F)(F)F)c3)c12 ; Cn1cc(-c2cc(-c3cnc(N)nc3)nc3sc(S(=O)C4CCC4)c(N)c23)cn1]

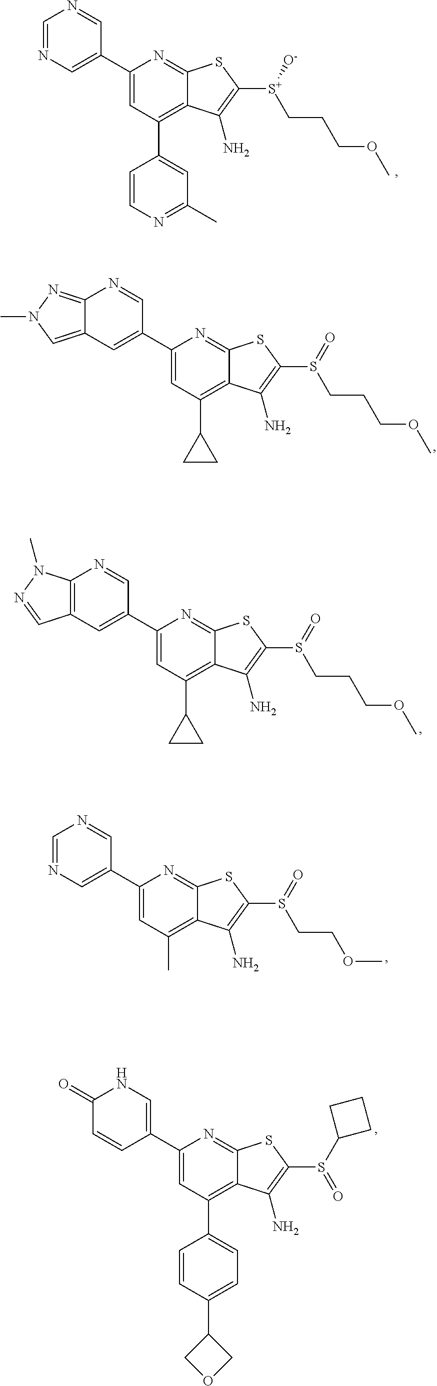

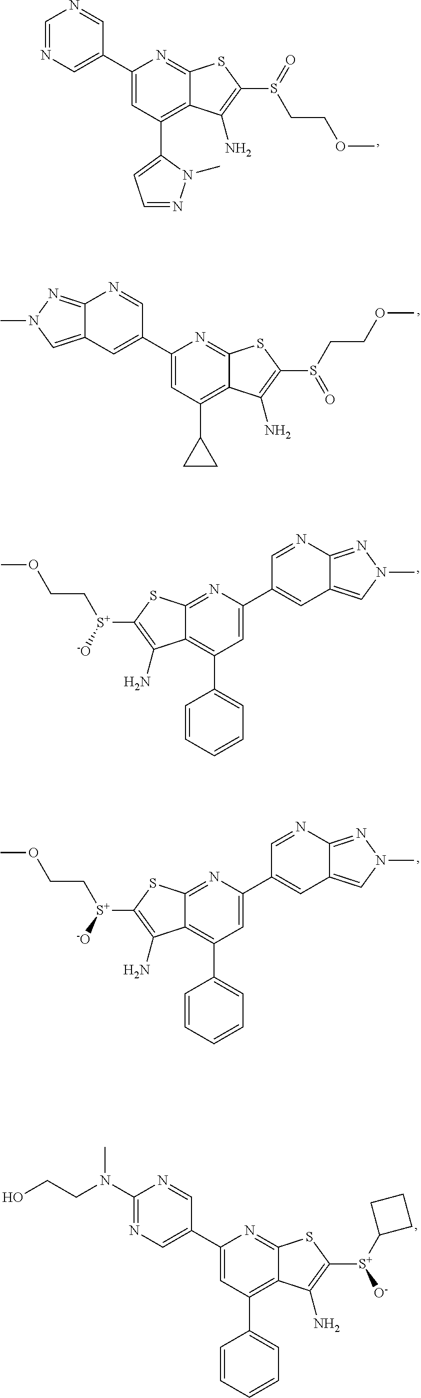

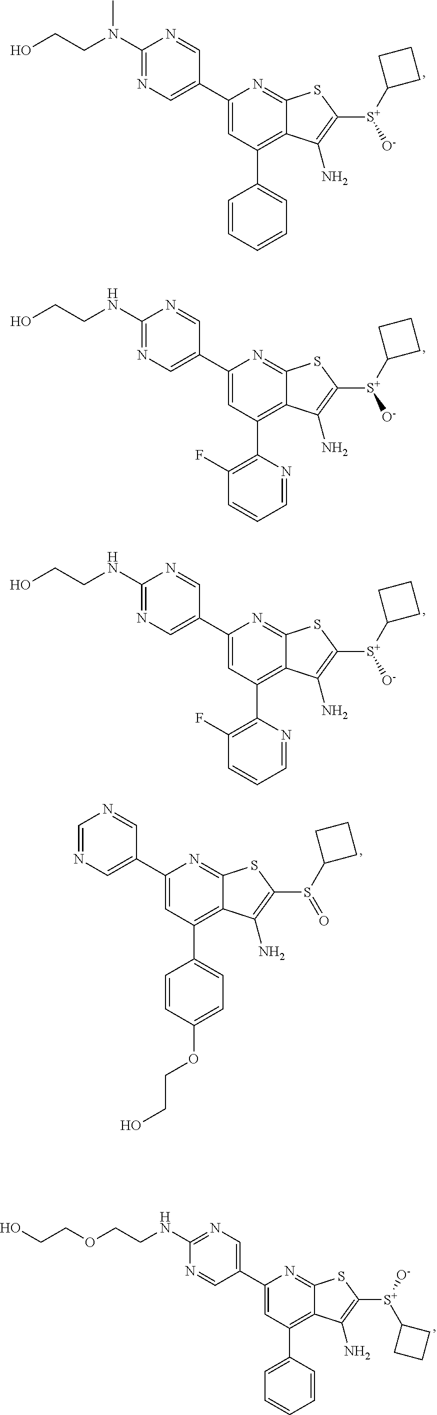 [SMILES: CN(CCO)c1ncc(-c2cc(-c3ccccc3)c3c(N)c([S@@+]([O-])C4CCC4)sc3n2)cn1 ; Nc1c([S@+]([O-])C2CCC2)sc2nc(-c3cnc(NCCO)nc3)cc(-c3ncccc3F)c12 ; Nc1c([S@@+]([O-])C2CCC2)sc2nc(-c3cnc(NCCO)nc3)cc(-c3ncccc3F)c12 ; Nc1c(S(=O)C2CCC2)sc2nc(-c3cncnc3)cc(-c3ccc(OCCO)cc3)c12 ; Nc1c([S@+]([O-])C2CCC2)sc2nc(-c3cnc(NCCOCCO)nc3)cc(-c3ccccc3)c12]

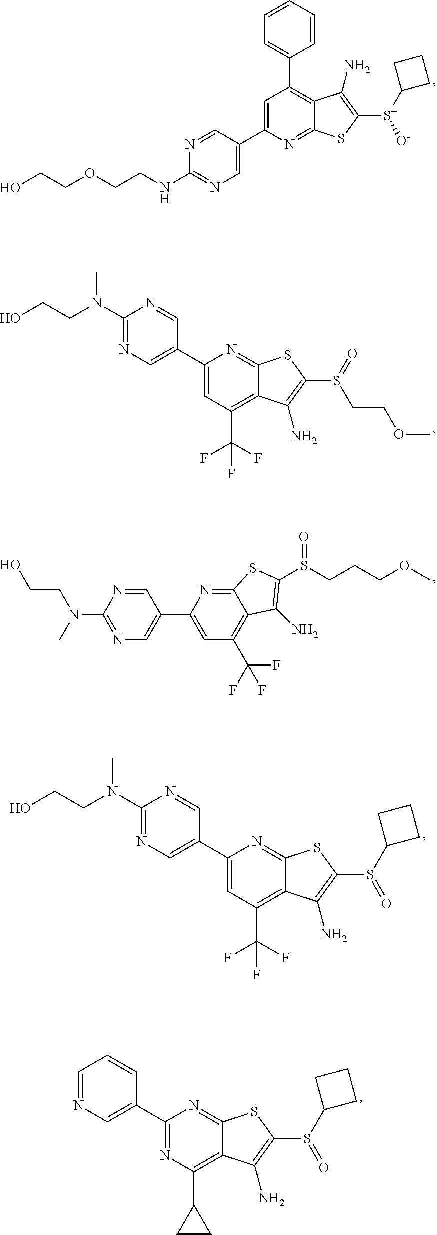 [SMILES: Nc1c([S@@+]([O-])C2CCC2)sc2nc(-c3cnc(NCCOCCO)nc3)cc(-c3ccccc3)c12 ; COCCS(=O)c1sc2nc(-c3cnc(N(C)CCO)nc3)cc(C(F)(F)F)c2c1N ; COCCCS(=O)c1sc2nc(-c3cnc(N(C)CCO)nc3)cc(C(F)(F)F)c2c1N ; CN(CCO)c1ncc(-c2cc(C(F)(F)F)c3c(N)c(S(=O)C4CCC4)sc3n2)cn1 ; Nc1c(S(=O)C2CCC2)sc2nc(-c3cccnc3)nc(C3CC3)c12]

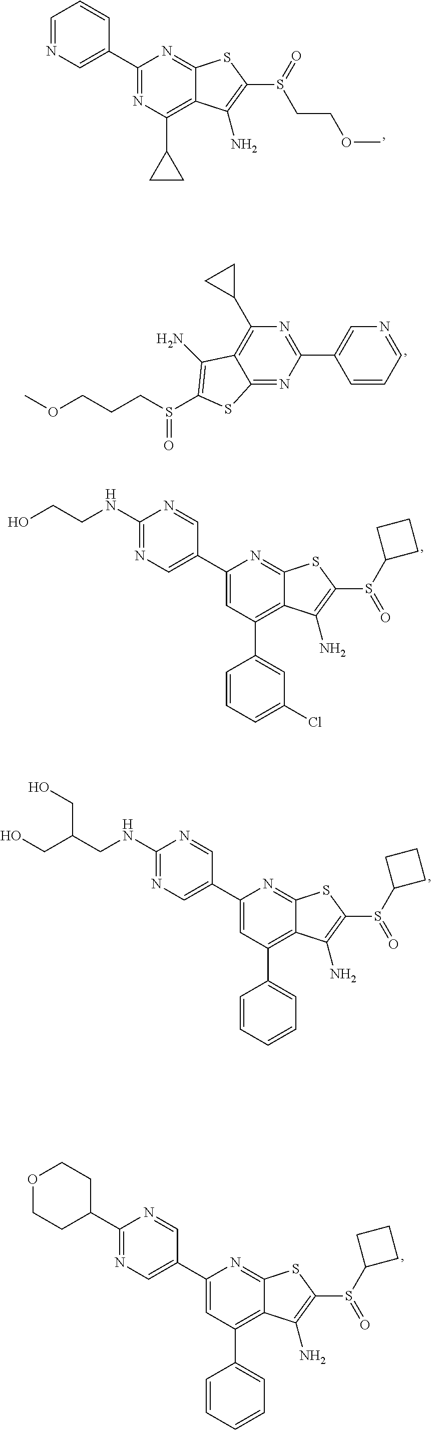

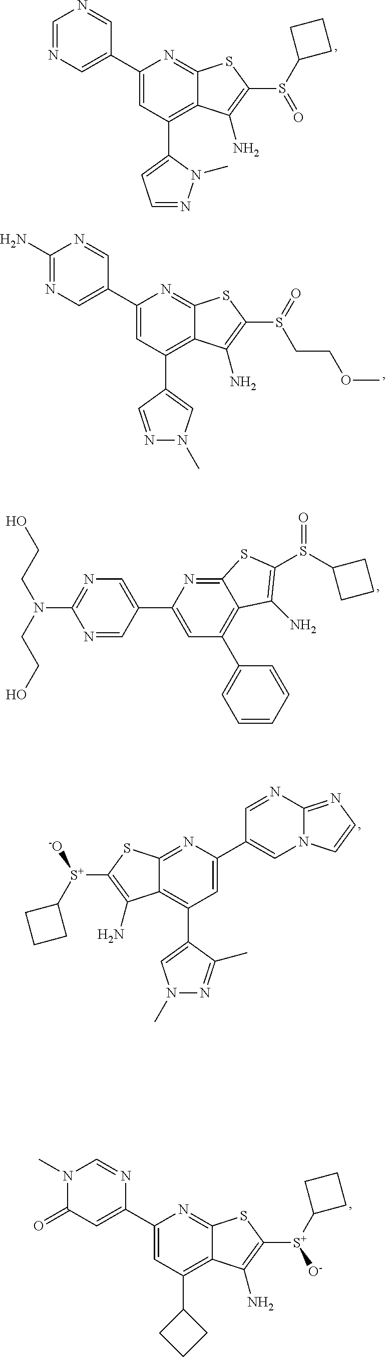

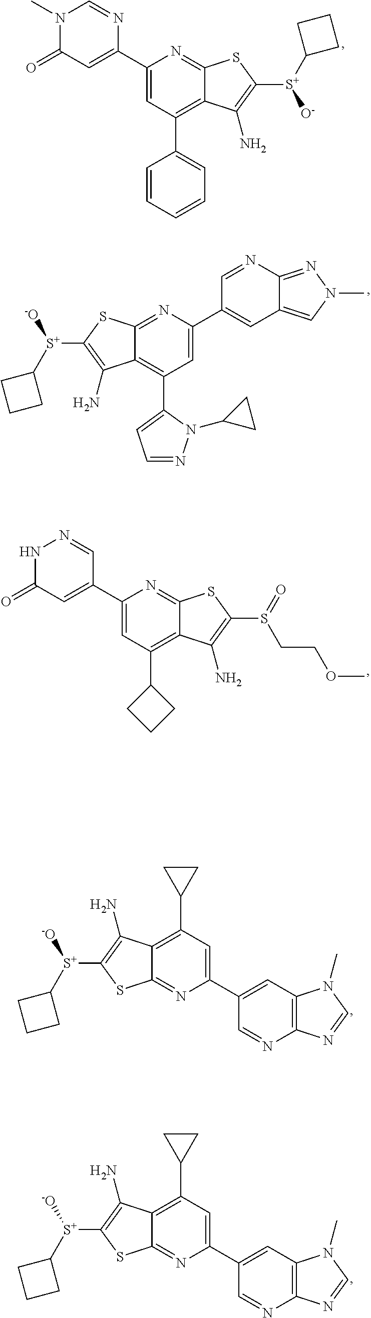

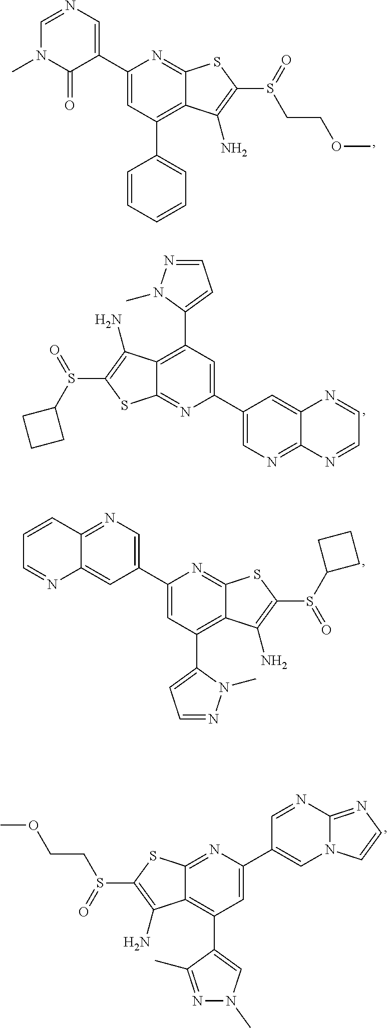

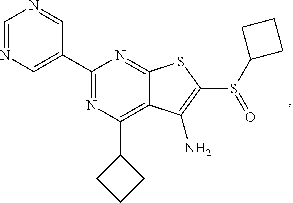 [SMILES: Nc1c(S(=O)C2CCC2)sc2nc(-c3cncnc3)nc(C3CCC3)c12]

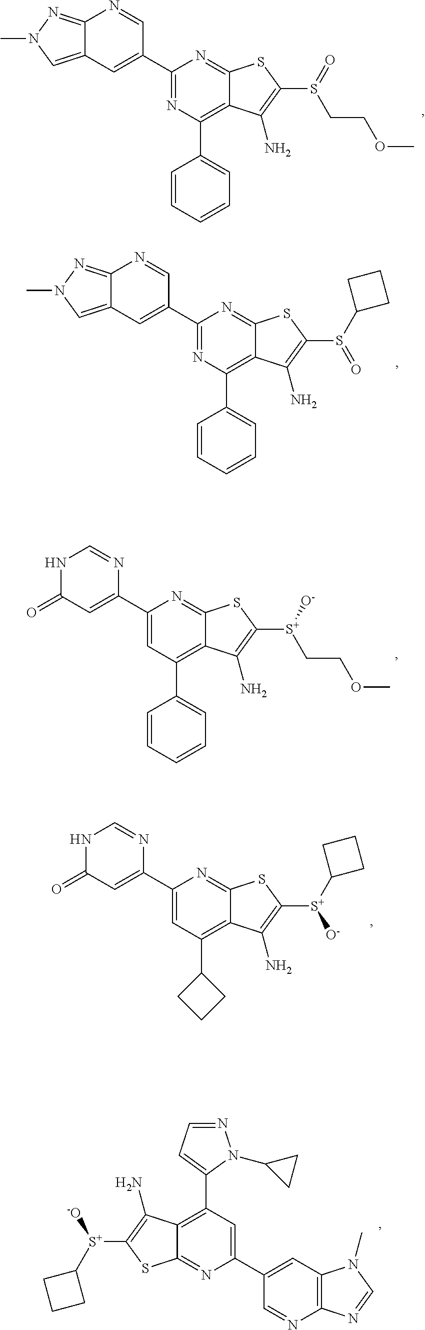

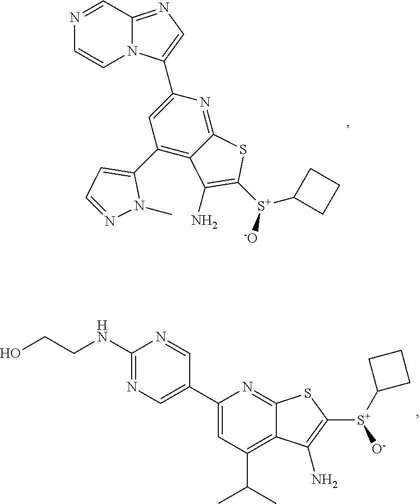

and molecules numbers 198-434 in Table 1, or pharmaceutically acceptable salts thereof, a tautomers thereof, and a solvates thereof.

In other embodiments, the 15-PGDH inhibitor is selected from compounds disclosed in any one of Examples 1-323, or pharmaceutically acceptable salts thereof, a tautomers thereof, and a solvates thereof.

In some embodiments, the 15-PGDH inhibitor having formula I, IA, II, III, IV, V, VI, VII, or VIII (i.e., formula I-VIII) is not a compound having the following formula:

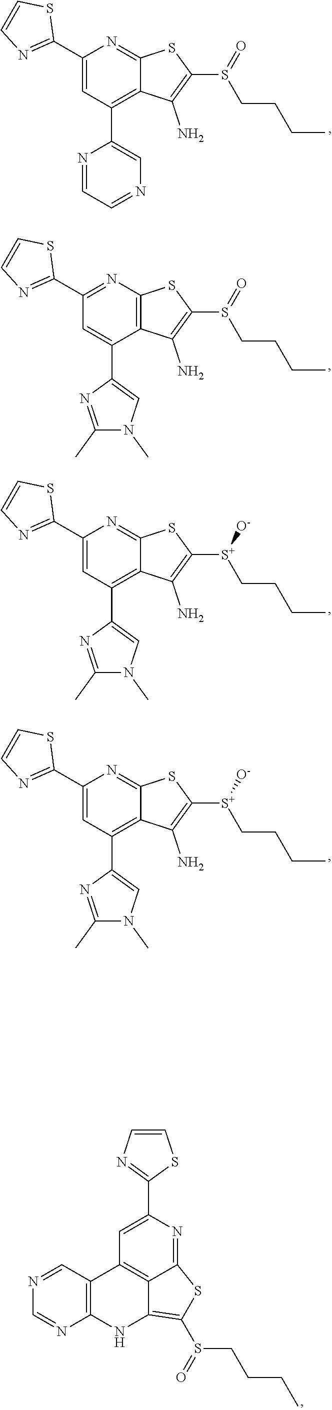

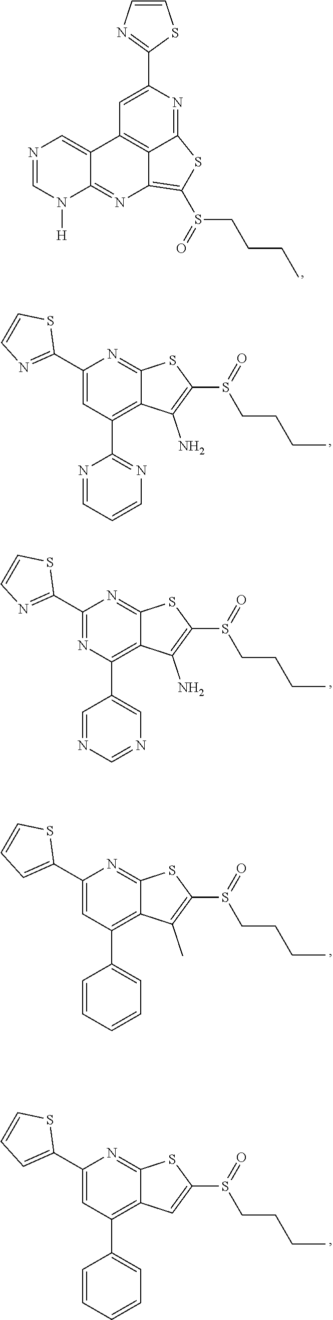 [SMILES: CCCCS(=O)c1sc2nc(-c3nccs3)cc3c4c(nc1c23)NC=NC=4 ; CCCCS(=O)c1sc2nc(-c3nccs3)cc(-c3ncccn3)c2c1N ; CCCCS(=O)c1sc2nc(-c3nccs3)nc(-c3cncnc3)c2c1N ; CCCCS(=O)c1sc2nc(-c3cccs3)cc(-c3ccccc3)c2c1C ; CCCCS(=O)c1cc2c(-c3ccccc3)cc(-c3cccs3)nc2s1]

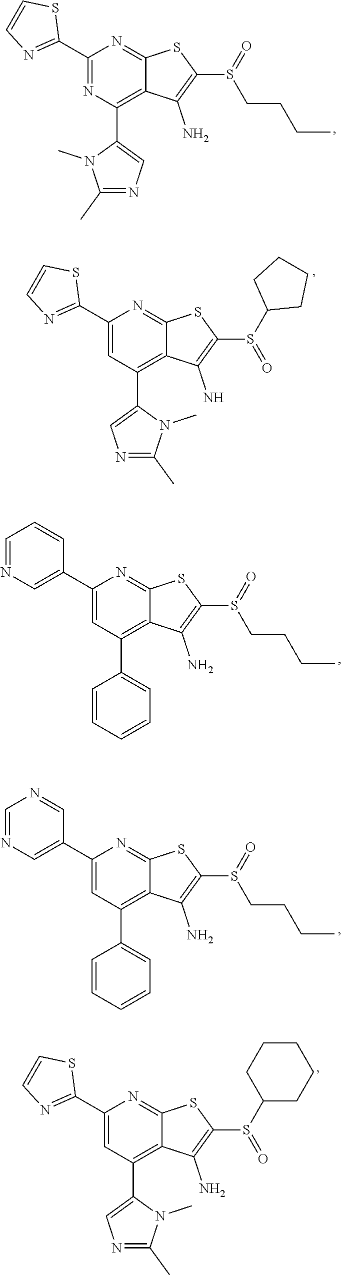 [SMILES: CCCCS(=O)c1sc2nc(-c3nccs3)nc(-c3cnc(C)n3C)c2c1N ; Cc1ncc(-c2cc(-c3nccs3)nc3sc(S(=O)C4CCCC4)c(N)c23)n1C ; CCCCS(=O)c1sc2nc(-c3cccnc3)cc(-c3ccccc3)c2c1N ; CCCCS(=O)c1sc2nc(-c3cncnc3)cc(-c3ccccc3)c2c1N ; Cc1ncc(-c2cc(-c3nccs3)nc3sc(S(=O)C4CCCCC4)c(N)c23)n1C]

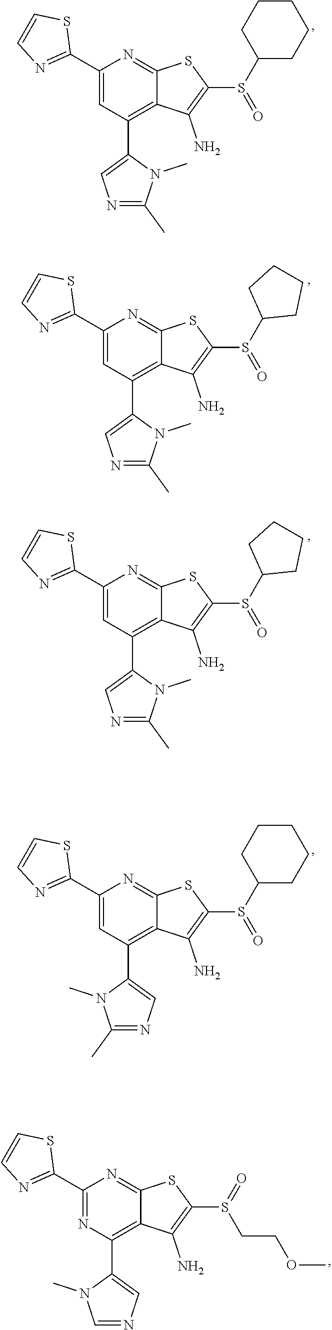 [SMILES: Cc1ncc(-c2cc(-c3nccs3)nc3sc(S(=O)C4CCCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCC4)c(N)c23)n1C ; Cc1ncc(-c2cc(-c3nccs3)nc3sc(S(=O)C4CCCCC4)c(N)c23)n1C ; COCCS(=O)c1sc2nc(-c3nccs3)nc(-c3cncn3C)c2c1N]

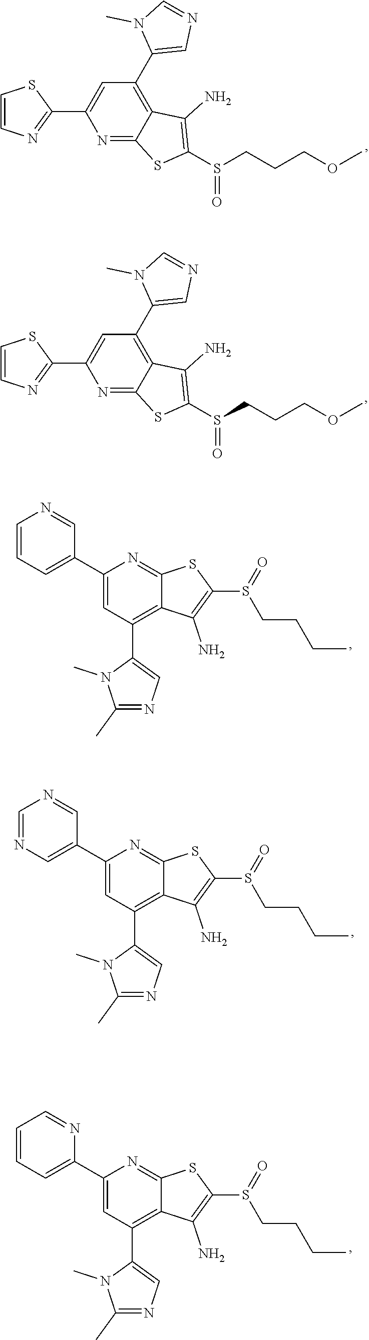 [SMILES: COCCCS(=O)c1sc2nc(-c3nccs3)cc(-c3cncn3C)c2c1N ; COCCC[S@@](=O)c1sc2nc(-c3nccs3)cc(-c3cncn3C)c2c1N ; CCCCS(=O)c1sc2nc(-c3cccnc3)cc(-c3cnc(C)n3C)c2c1N ; CCCCS(=O)c1sc2nc(-c3cncnc3)cc(-c3cnc(C)n3C)c2c1N ; CCCCS(=O)c1sc2nc(-c3ccccn3)cc(-c3cnc(C)n3C)c2c1N]

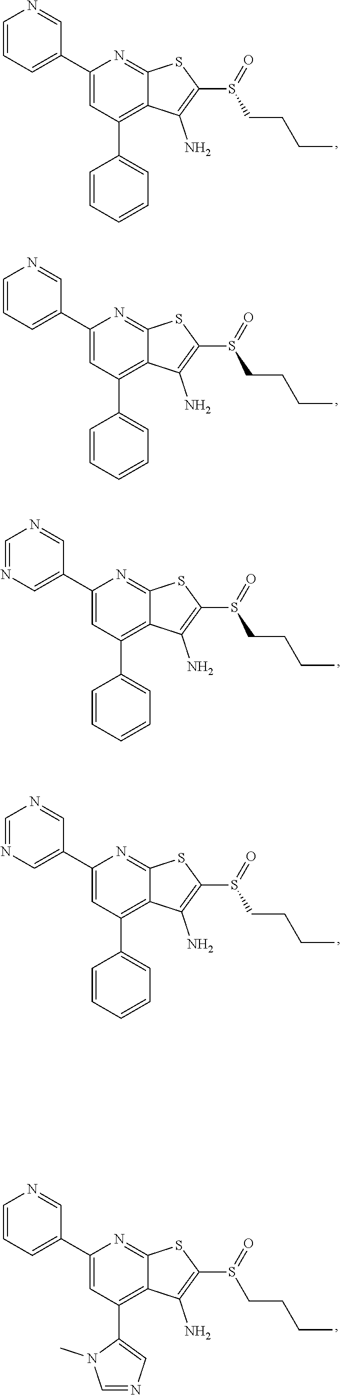 [SMILES: CCCC[S@@](=O)c1sc2nc(-c3cccnc3)cc(-c3ccccc3)c2c1N ; CCCC[S@](=O)c1sc2nc(-c3cccnc3)cc(-c3ccccc3)c2c1N ; CCCC[S@](=O)c1sc2nc(-c3cncnc3)cc(-c3ccccc3)c2c1N ; CCCC[S@@](=O)c1sc2nc(-c3cncnc3)cc(-c3ccccc3)c2c1N ; CCCCS(=O)c1sc2nc(-c3cccnc3)cc(-c3cncn3C)c2c1N]

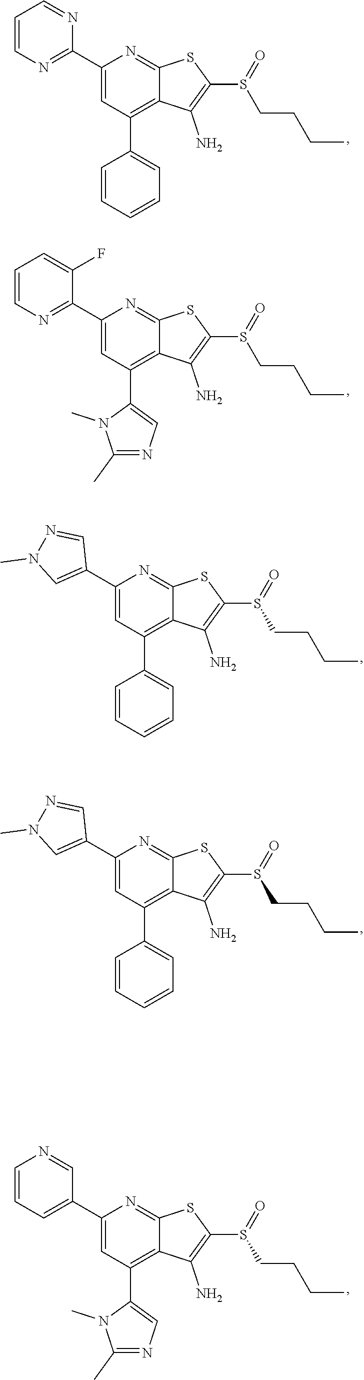

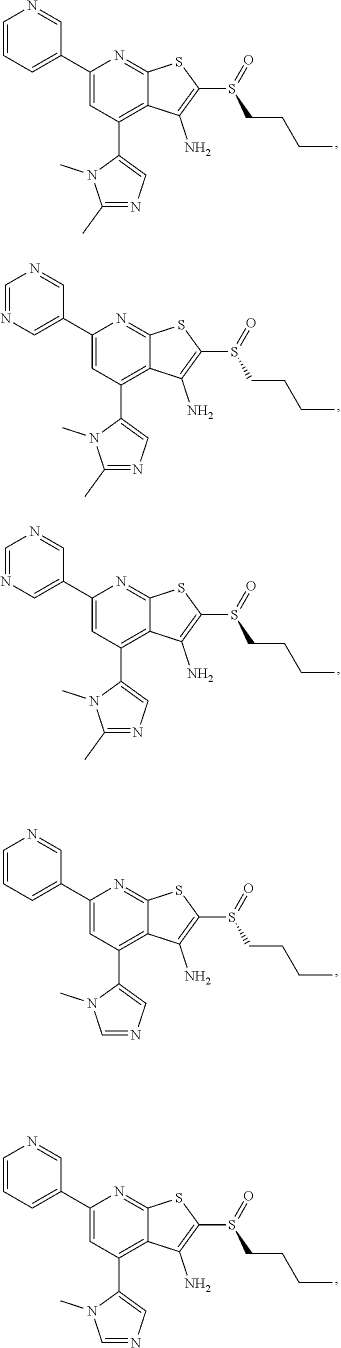 [SMILES: CCCC[S@](=O)c1sc2nc(-c3cccnc3)cc(-c3cnc(C)n3C)c2c1N ; CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N ; CCCC[S@](=O)c1sc2nc(-c3cccnc3)cc(-c3cncn3C)c2c1N]

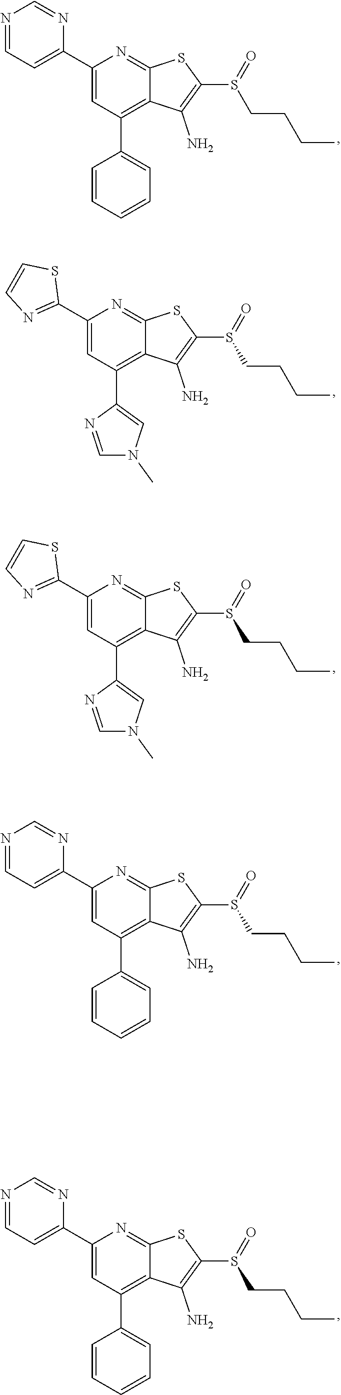 [SMILES: CCCCS(=O)c1sc2nc(-c3ccncn3)cc(-c3ccccc3)c2c1N ; CCCC[S@@](=O)c1sc2nc(-c3nccs3)cc(-c3cn(C)cn3)c2c1N ; CCCC[S@](=O)c1sc2nc(-c3nccs3)cc(-c3cn(C)cn3)c2c1N ; CCCC[S@@](=O)c1sc2nc(-c3ccncn3)cc(-c3ccccc3)c2c1N ; CCCC[S@](=O)c1sc2nc(-c3ccncn3)cc(-c3ccccc3)c2c1N]

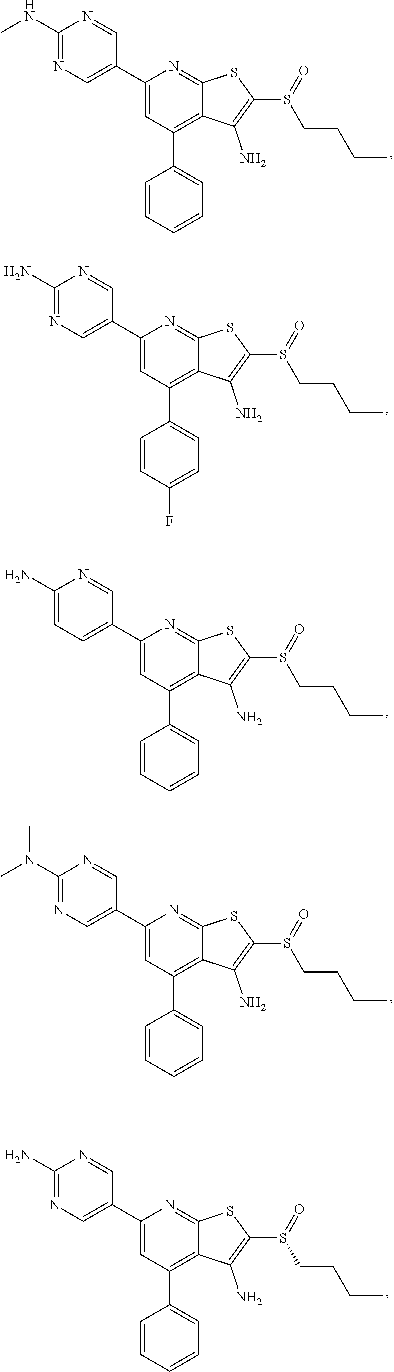

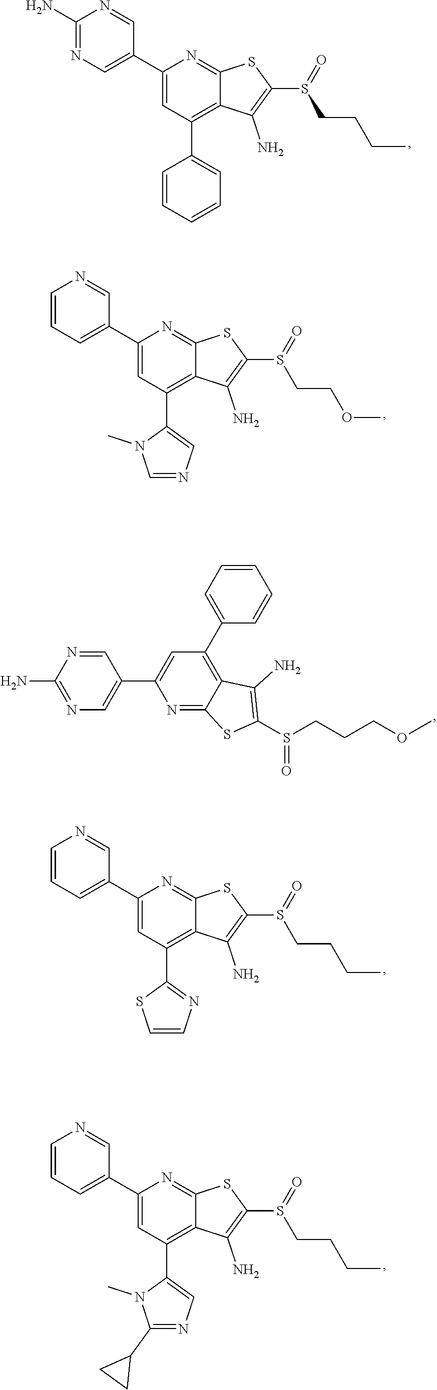 [SMILES: CCCC[S@](=O)c1sc2nc(-c3cnc(N)nc3)cc(-c3ccccc3)c2c1N ; COCCS(=O)c1sc2nc(-c3cccnc3)cc(-c3cncn3C)c2c1N ; COCCCS(=O)c1sc2nc(-c3cnc(N)nc3)cc(-c3ccccc3)c2c1N ; CCCCS(=O)c1sc2nc(-c3cccnc3)cc(-c3nccs3)c2c1N ; CCCCS(=O)c1sc2nc(-c3cccnc3)cc(-c3cnc(C4CC4)n3C)c2c1N]

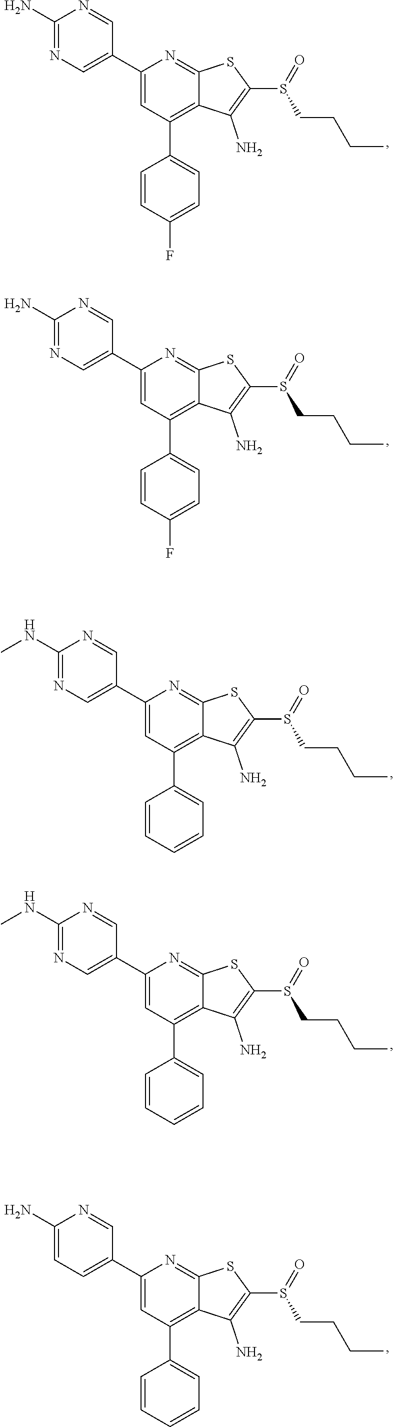

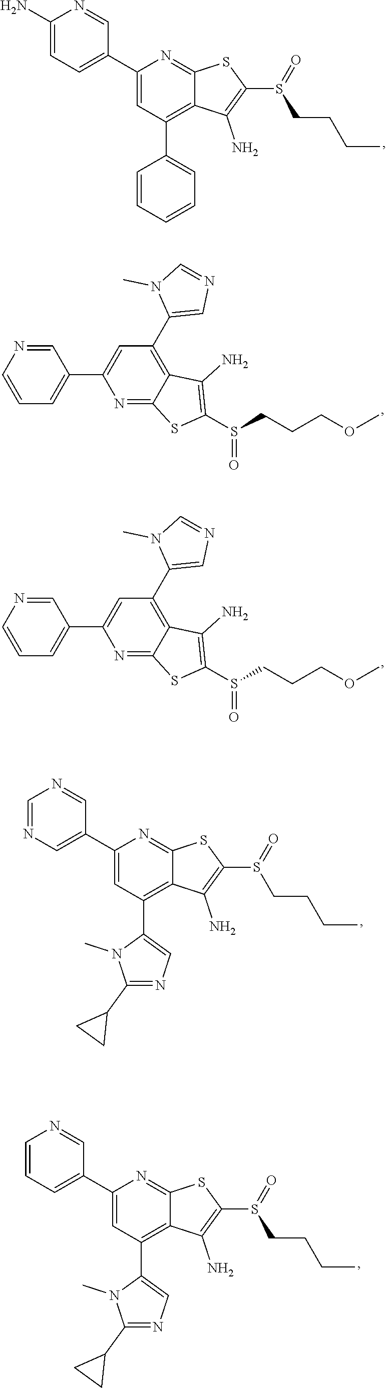

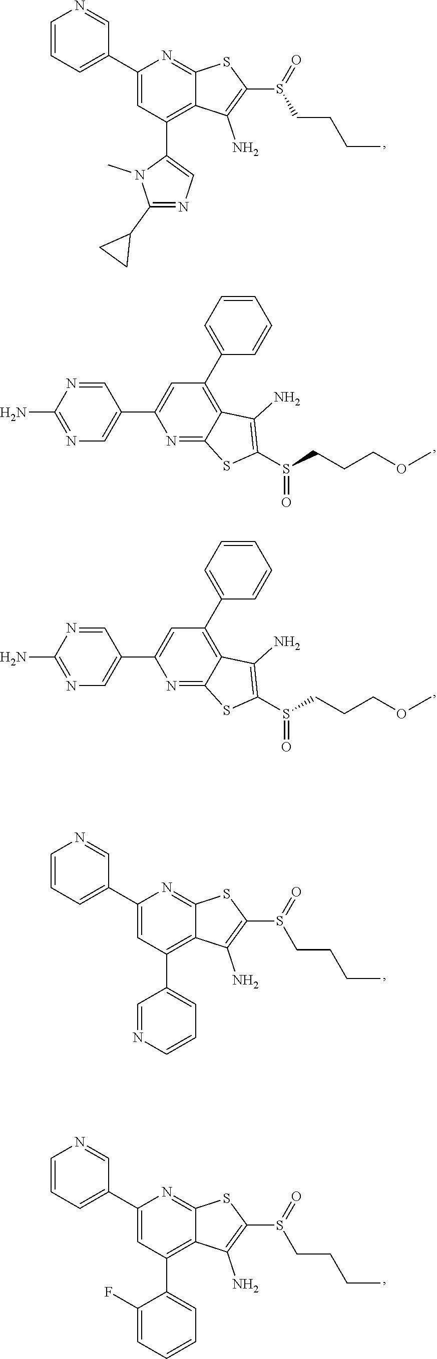 [SMILES: CCCC[S@@](=O)c1sc2nc(-c3cccnc3)cc(-c3cnc(C4CC4)n3C)c2c1N ; COCCC[S@@](=O)c1sc2nc(-c3cnc(N)nc3)cc(-c3ccccc3)c2c1N ; COCCC[S@](=O)c1sc2nc(-c3cnc(N)nc3)cc(-c3ccccc3)c2c1N ; CCCCS(=O)c1sc2nc(-c3cccnc3)cc(-c3cccnc3)c2c1N ; CCCCS(=O)c1sc2nc(-c3cccnc3)cc(-c3ccccc3F)c2c1N]

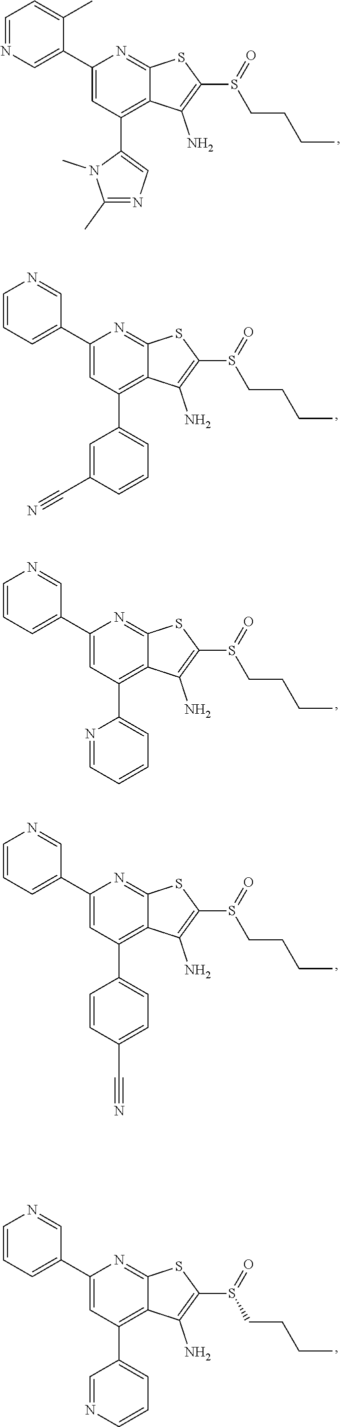 [SMILES: CCCCS(=O)c1sc2nc(-c3cnccc3C)cc(-c3cnc(C)n3C)c2c1N ; CCCCS(=O)c1sc2nc(-c3cccnc3)cc(-c3cccc(C#N)c3)c2c1N ; CCCCS(=O)c1sc2nc(-c3cccnc3)cc(-c3ccccn3)c2c1N ; CCCCS(=O)c1sc2nc(-c3cccnc3)cc(-c3ccc(C#N)cc3)c2c1N ; CCCC[S@@](=O)c1sc2nc(-c3cccnc3)cc(-c3cccnc3)c2c1N]

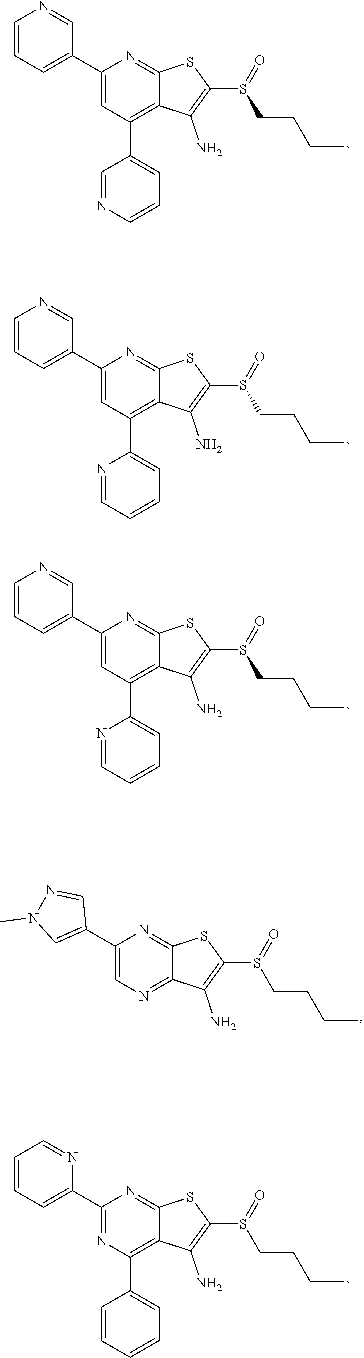

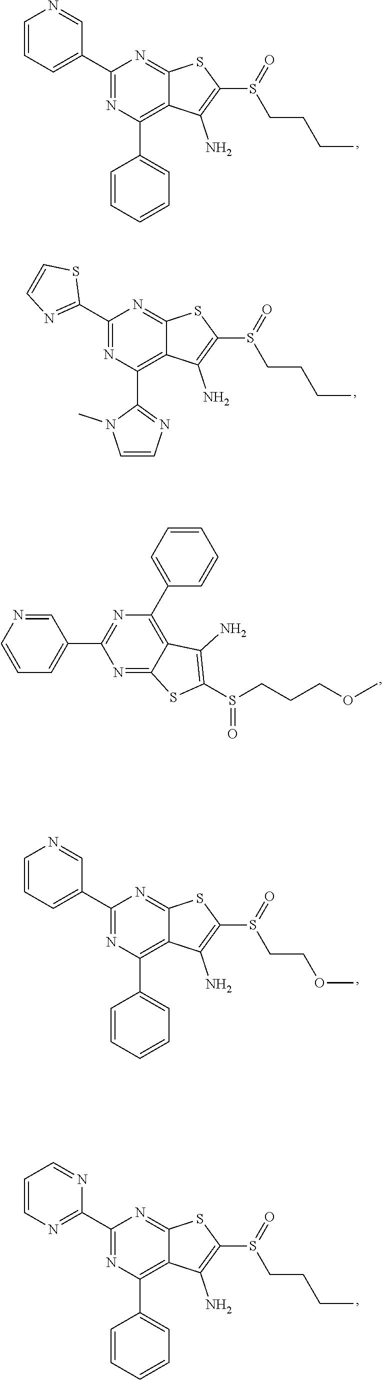 [SMILES: CCCCS(=O)c1sc2nc(-c3cccnc3)nc(-c3ccccc3)c2c1N ; CCCCS(=O)c1sc2nc(-c3nccs3)nc(-c3nccn3C)c2c1N ; COCCCS(=O)c1sc2nc(-c3cccnc3)nc(-c3ccccc3)c2c1N ; COCCS(=O)c1sc2nc(-c3cccnc3)nc(-c3ccccc3)c2c1N ; CCCCS(=O)c1sc2nc(-c3ncccn3)nc(-c3ccccc3)c2c1N]

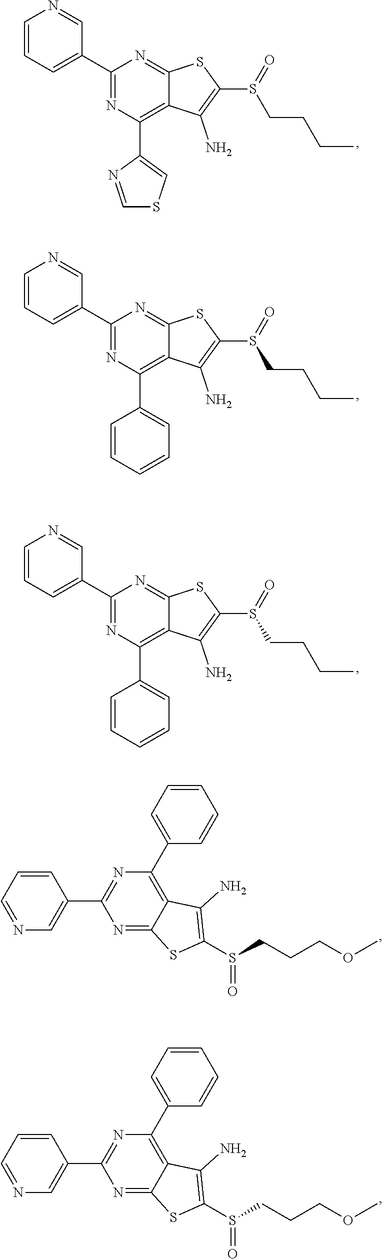 [SMILES: CCCCS(=O)c1sc2nc(-c3cccnc3)nc(-c3cscn3)c2c1N ; CCCC[S@](=O)c1sc2nc(-c3cccnc3)nc(-c3ccccc3)c2c1N ; CCCC[S@@](=O)c1sc2nc(-c3cccnc3)nc(-c3ccccc3)c2c1N ; COCCC[S@@](=O)c1sc2nc(-c3cccnc3)nc(-c3ccccc3)c2c1N ; COCCC[S@](=O)c1sc2nc(-c3cccnc3)nc(-c3ccccc3)c2c1N]

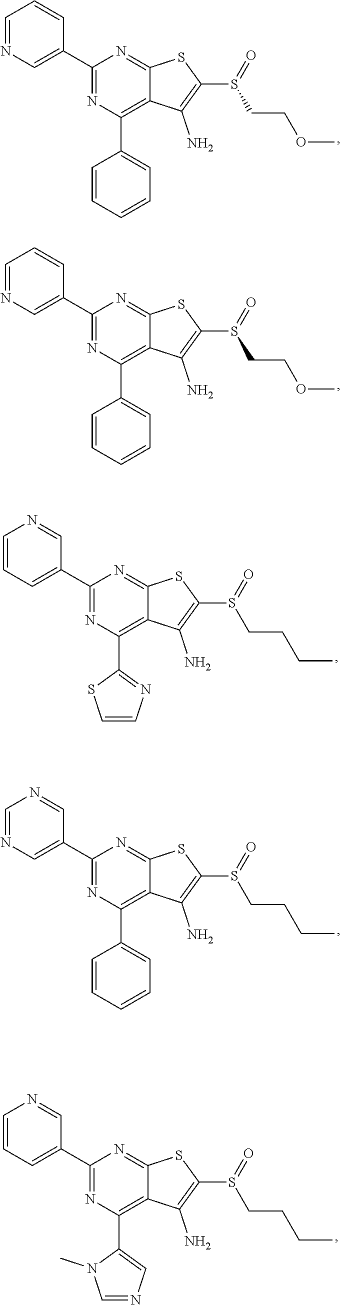 [SMILES: COCC[S@@](=O)c1sc2nc(-c3cccnc3)nc(-c3ccccc3)c2c1N ; COCC[S@](=O)c1sc2nc(-c3cccnc3)nc(-c3ccccc3)c2c1N ; CCCCS(=O)c1sc2nc(-c3cccnc3)nc(-c3nccs3)c2c1N ; CCCCS(=O)c1sc2nc(-c3cncnc3)nc(-c3ccccc3)c2c1N ; CCCCS(=O)c1sc2nc(-c3cccnc3)nc(-c3cncn3C)c2c1N]

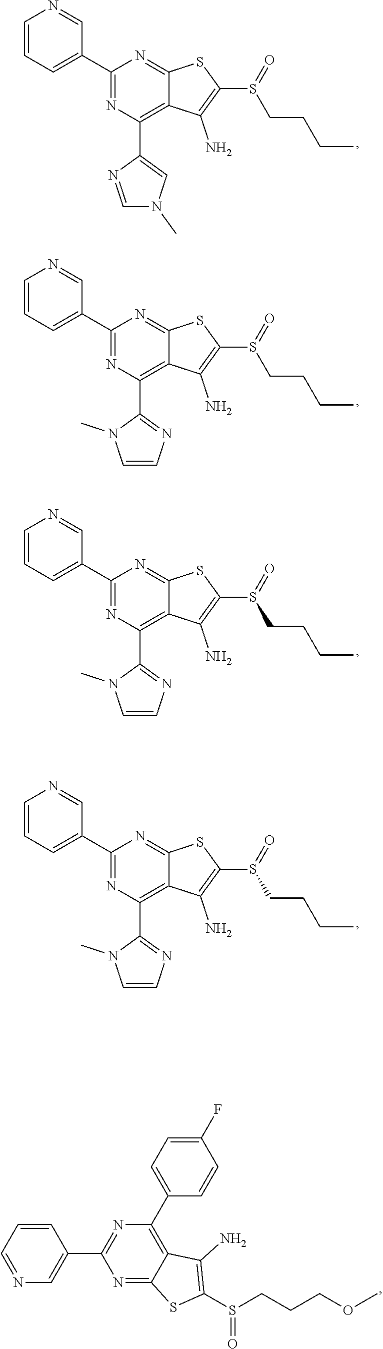 [SMILES: CCCCS(=O)c1sc2nc(-c3cccnc3)nc(-c3cn(C)cn3)c2c1N ; CCCCS(=O)c1sc2nc(-c3cccnc3)nc(-c3nccn3C)c2c1N ; CCCC[S@](=O)c1sc2nc(-c3cccnc3)nc(-c3nccn3C)c2c1N ; CCCC[S@@](=O)c1sc2nc(-c3cccnc3)nc(-c3nccn3C)c2c1N ; COCCCS(=O)c1sc2nc(-c3cccnc3)nc(-c3ccc(F)cc3)c2c1N]

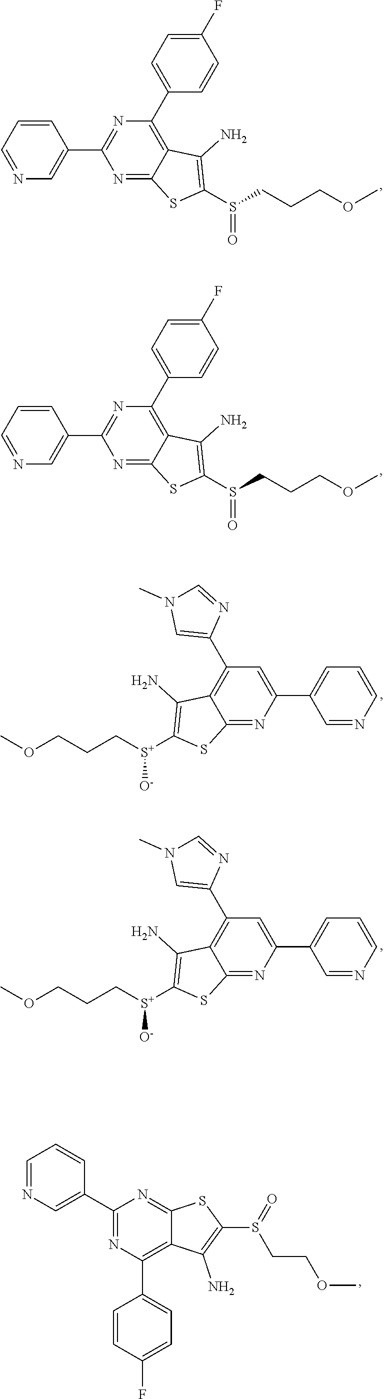

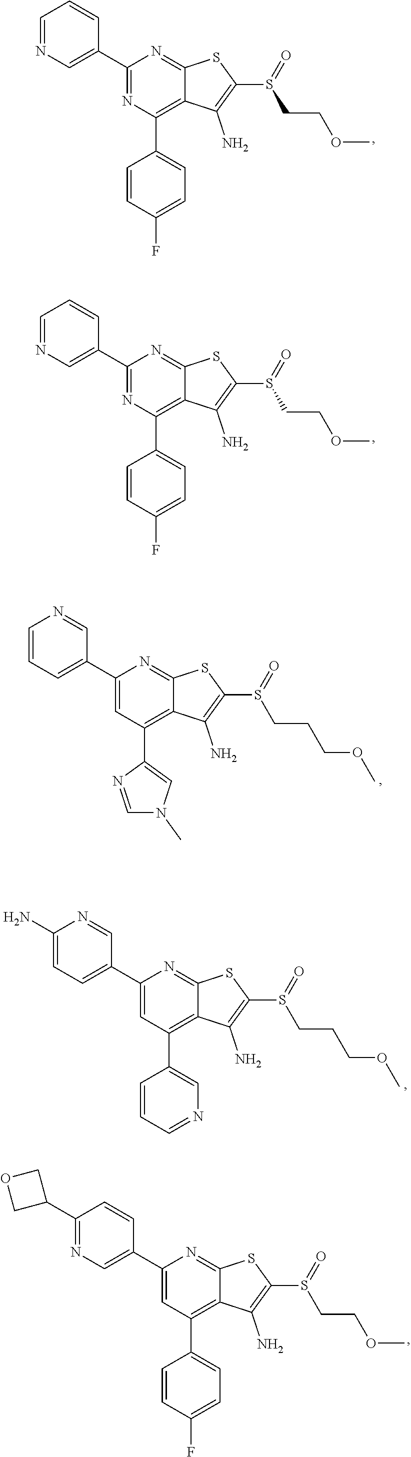 [SMILES: COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]

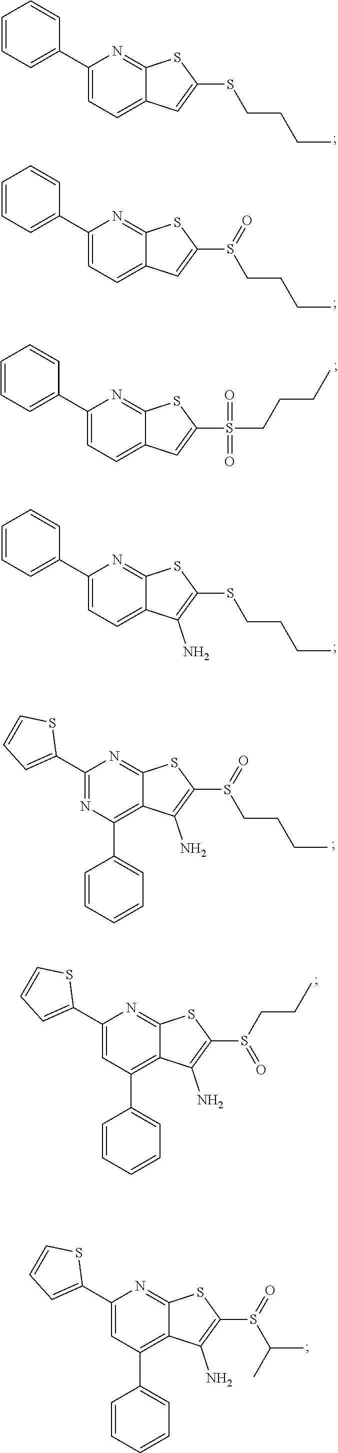

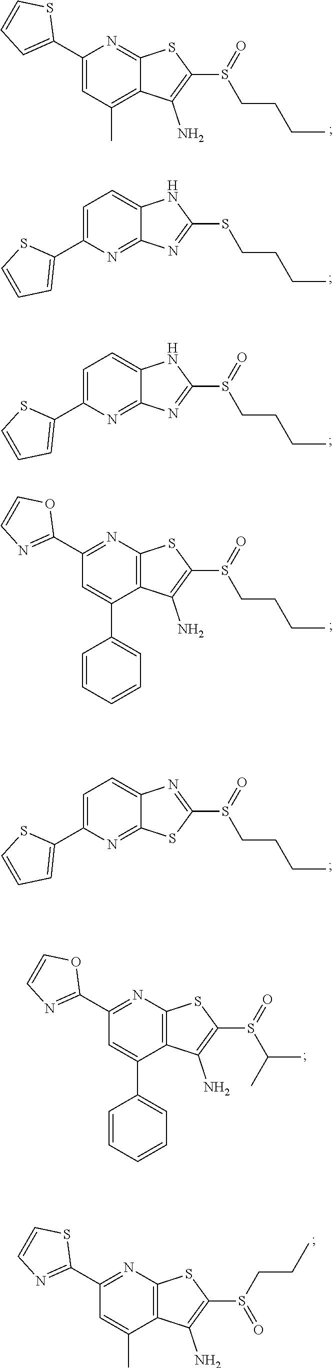

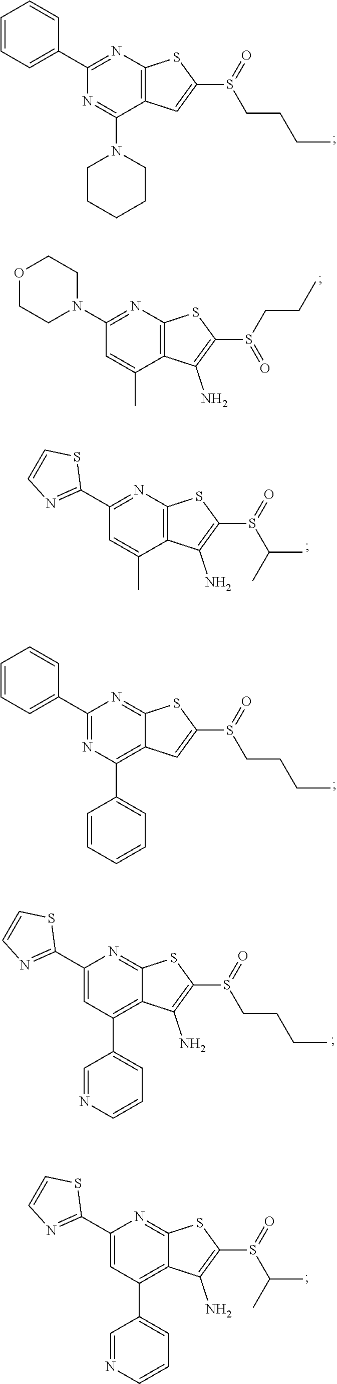 [SMILES: CCCCS(=O)c1cc2c(N3CCCCC3)nc(-c3ccccc3)nc2s1 ; CCCS(=O)c1sc2nc(N3CCOCC3)cc(C)c2c1N ; Cc1cc(-c2nccs2)nc2sc(S(=O)C(C)C)c(N)c12 ; CCCCS(=O)c1cc2c(-c3ccccc3)nc(-c3ccccc3)nc2s1 ; CCCCS(=O)c1sc2nc(-c3nccs3)cc(-c3cccnc3)c2c1N ; CC(C)S(=O)c1sc2nc(-c3nccs3)cc(-c3cccnc3)c2c1N]

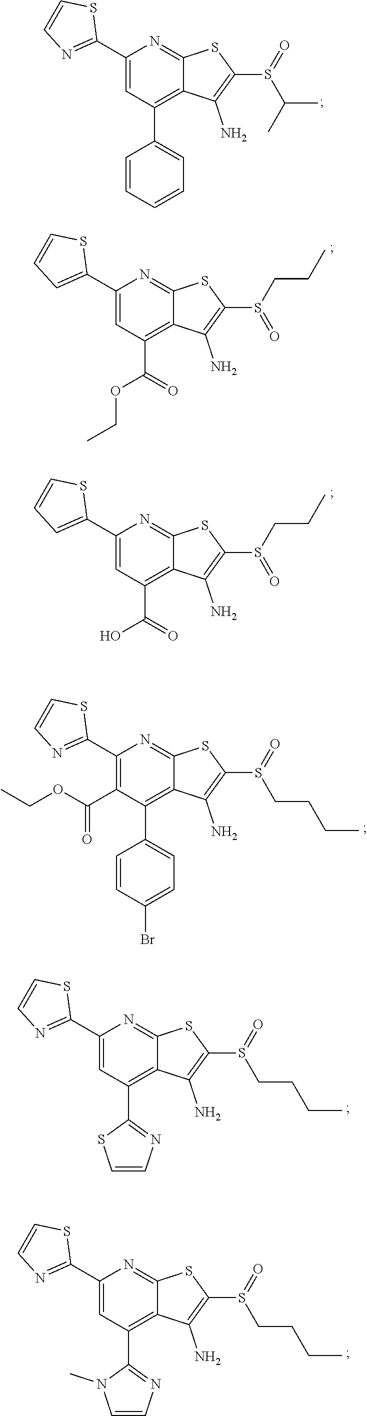

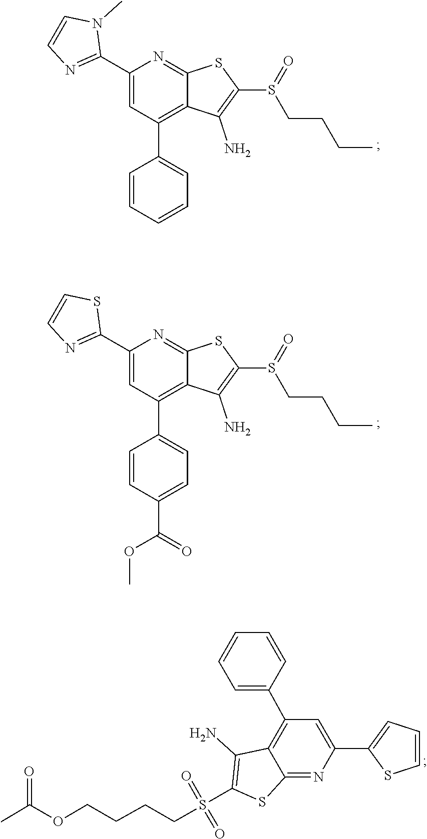

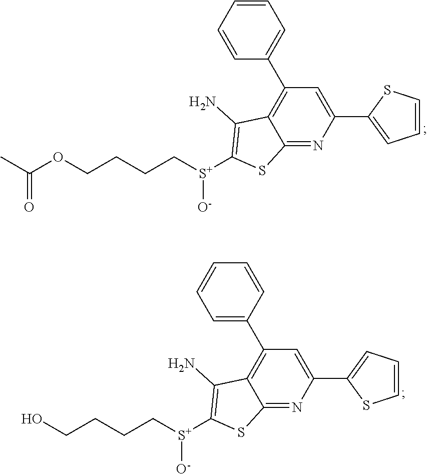 [SMILES: CC(=O)OCCCC[S+]([O-])c1sc2nc(-c3cccs3)cc(-c3ccccc3)c2c1N ; Nc1c([S+]([O-])CCCCO)sc2nc(-c3cccs3)cc(-c3ccccc3)c12]

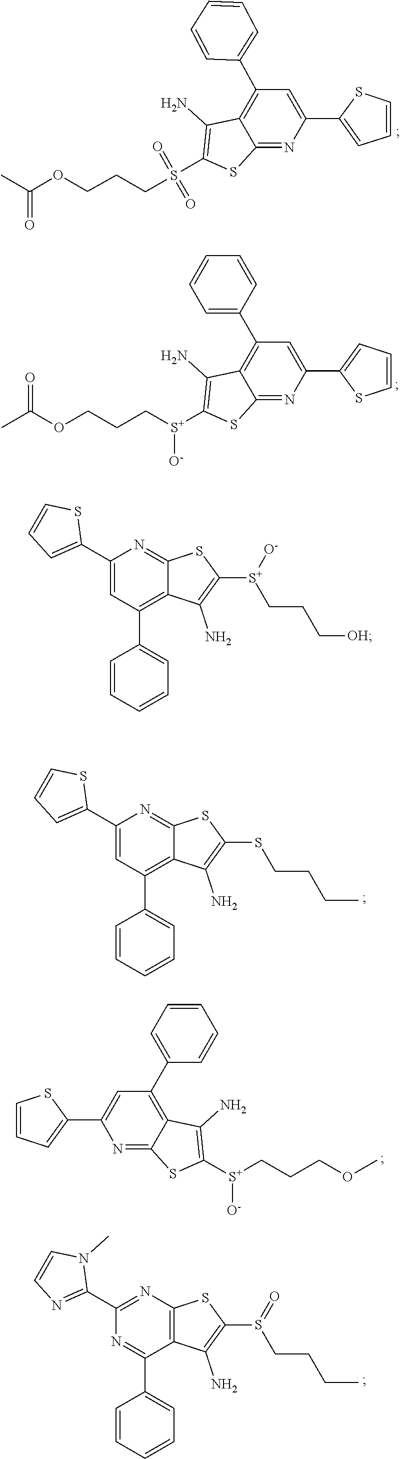

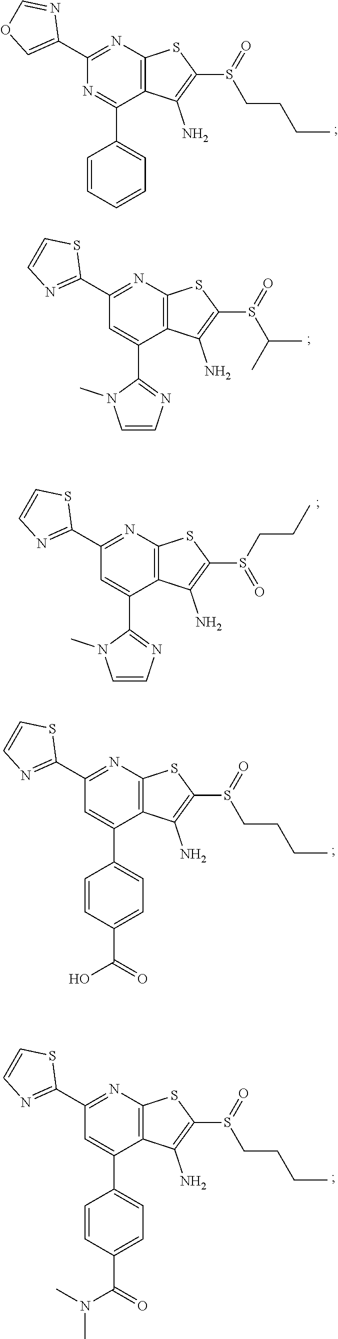 [SMILES: CCCCS(=O)c1sc2nc(-c3cocn3)nc(-c3ccccc3)c2c1N ; CC(C)S(=O)c1sc2nc(-c3nccs3)cc(-c3nccn3C)c2c1N ; CCCS(=O)c1sc2nc(-c3nccs3)cc(-c3nccn3C)c2c1N ; CCCCS(=O)c1sc2nc(-c3nccs3)cc(-c3ccc(C(=O)O)cc3)c2c1N ; CCCCS(=O)c1sc2nc(-c3nccs3)cc(-c3ccc(C(=O)N(C)C)cc3)c2c1N]

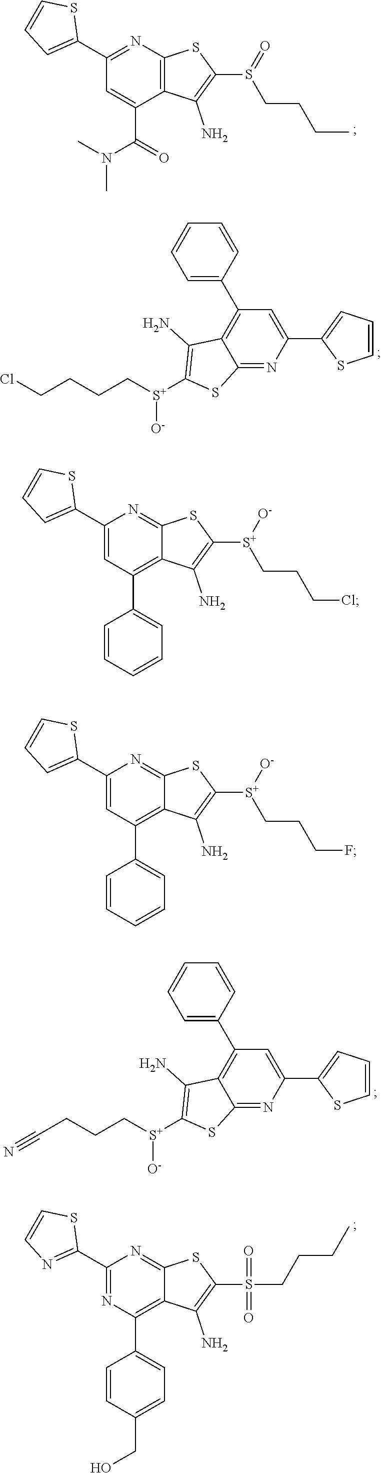 [SMILES: CCCCS(=O)c1sc2nc(-c3cccs3)cc(C(=O)N(C)C)c2c1N ; Nc1c([S+]([O-])CCCCCl)sc2nc(-c3cccs3)cc(-c3ccccc3)c12 ; Nc1c([S+]([O-])CCCCl)sc2nc(-c3cccs3)cc(-c3ccccc3)c12 ; Nc1c([S+]([O-])CCCF)sc2nc(-c3cccs3)cc(-c3ccccc3)c12 ; N#CCCC[S+]([O-])c1sc2nc(-c3cccs3)cc(-c3ccccc3)c2c1N ; CCCCS(=O)(=O)c1sc2nc(-c3nccs3)nc(-c3ccc(CO)cc3)c2c1N]

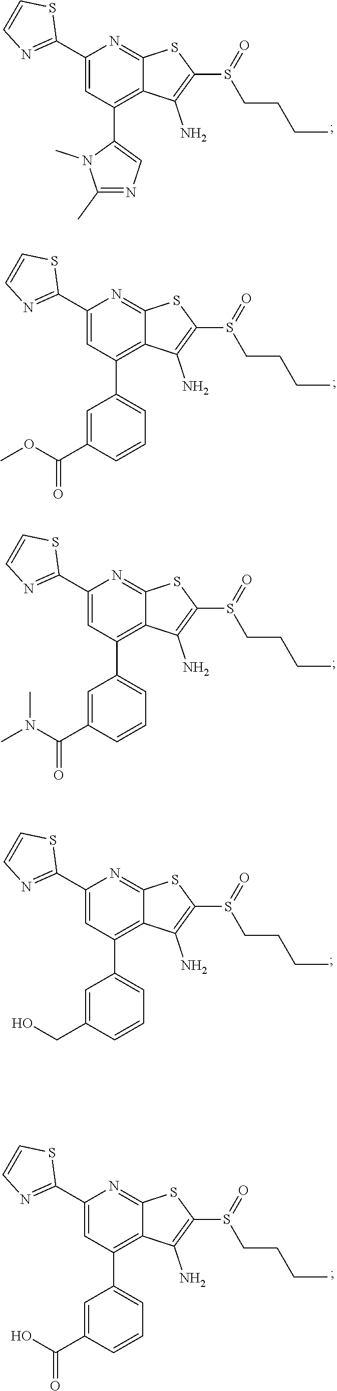

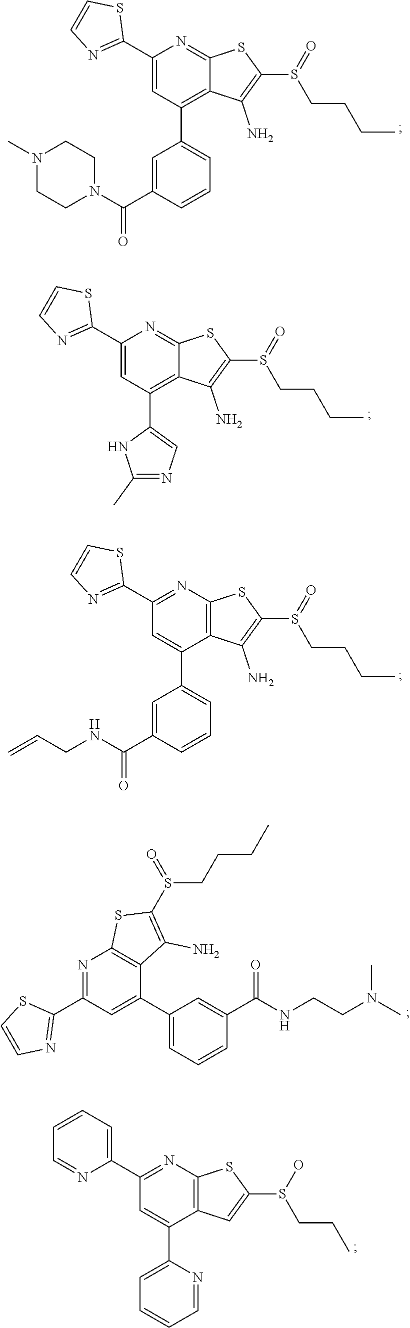

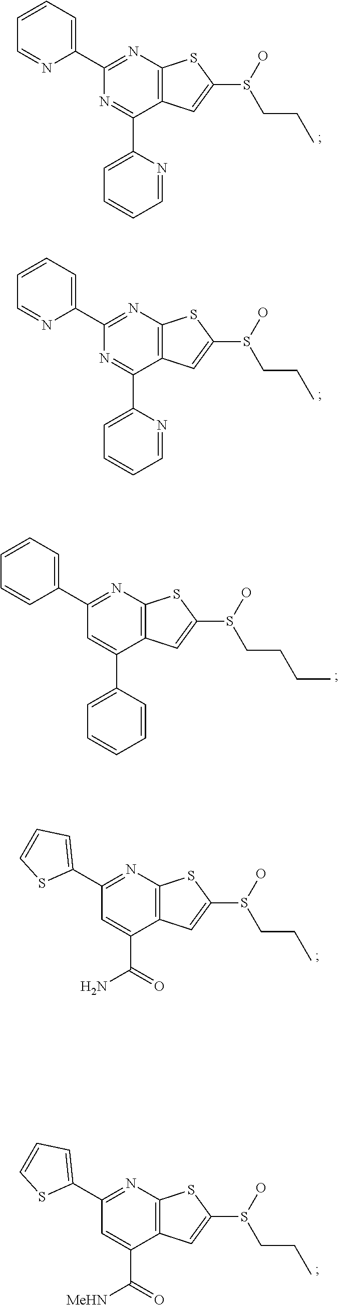

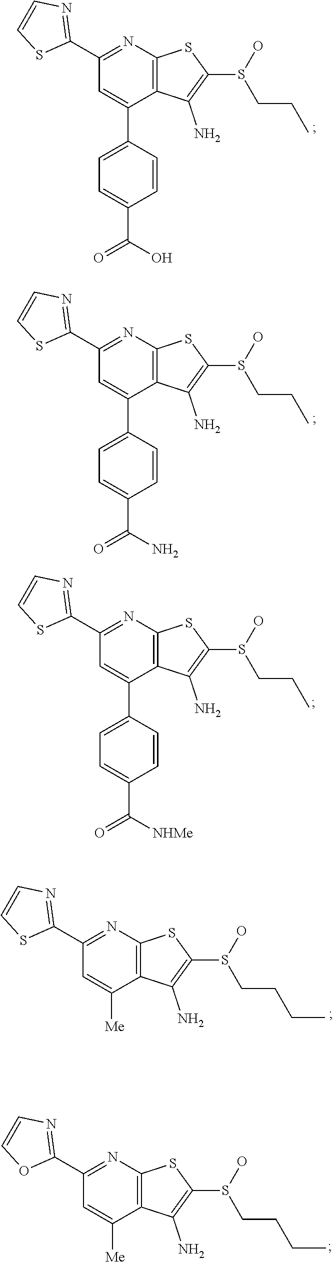

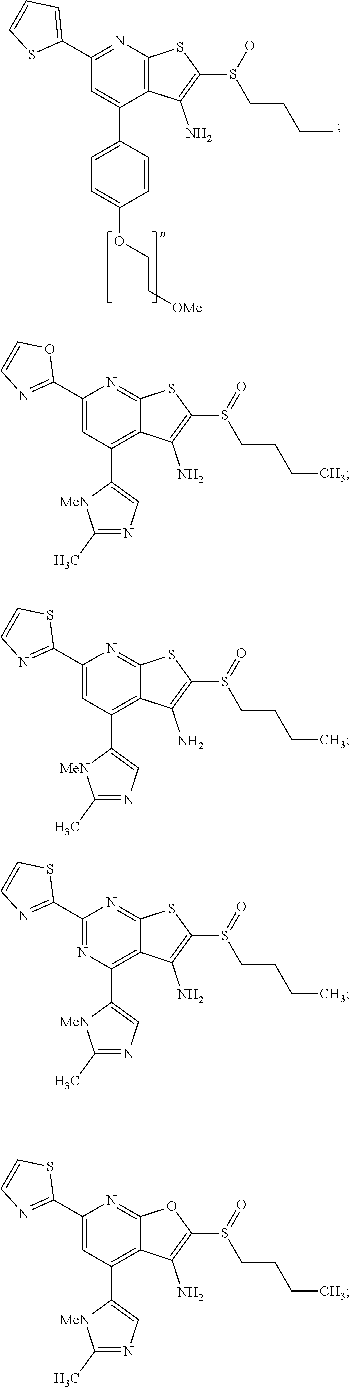

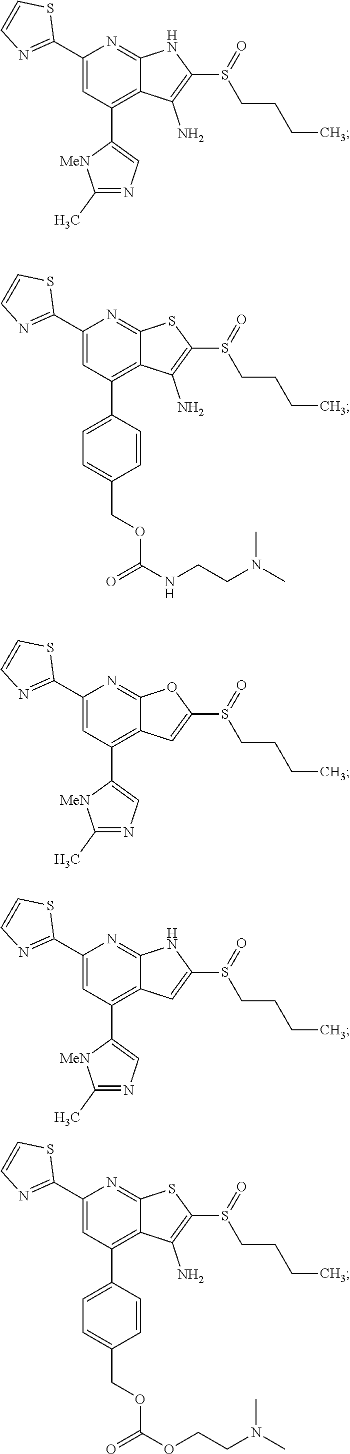

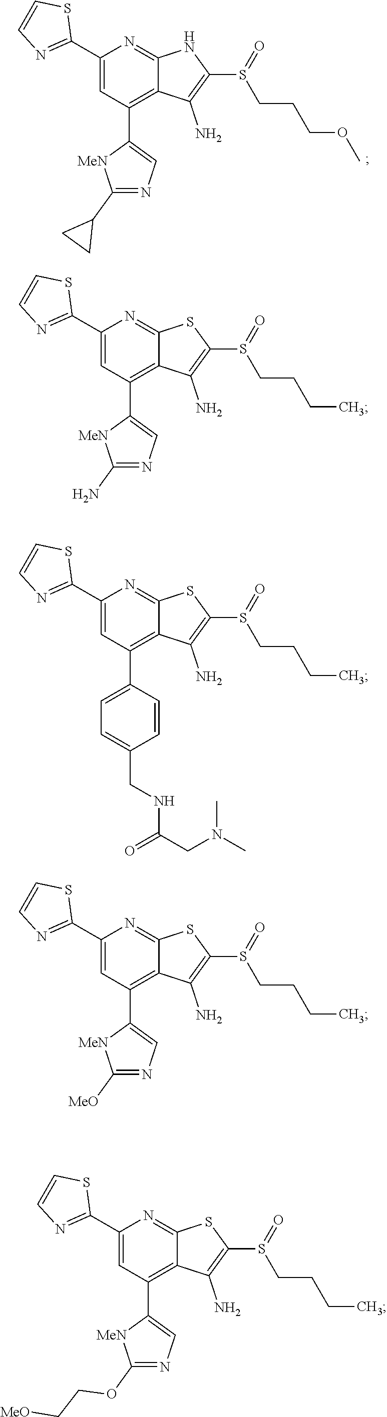

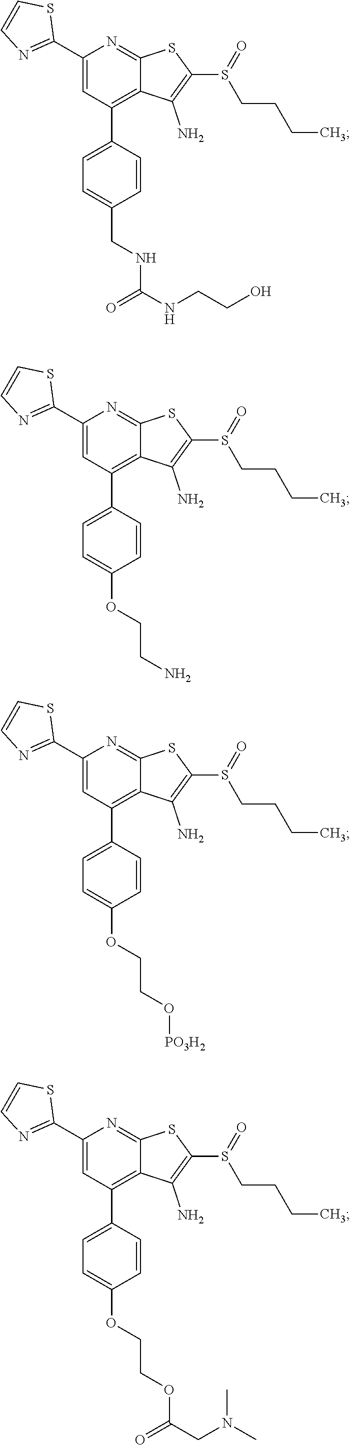

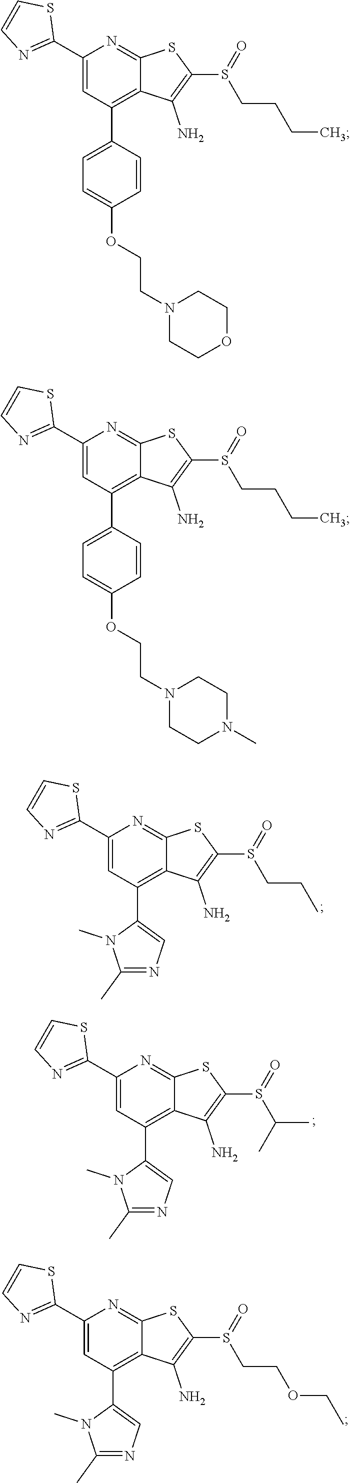

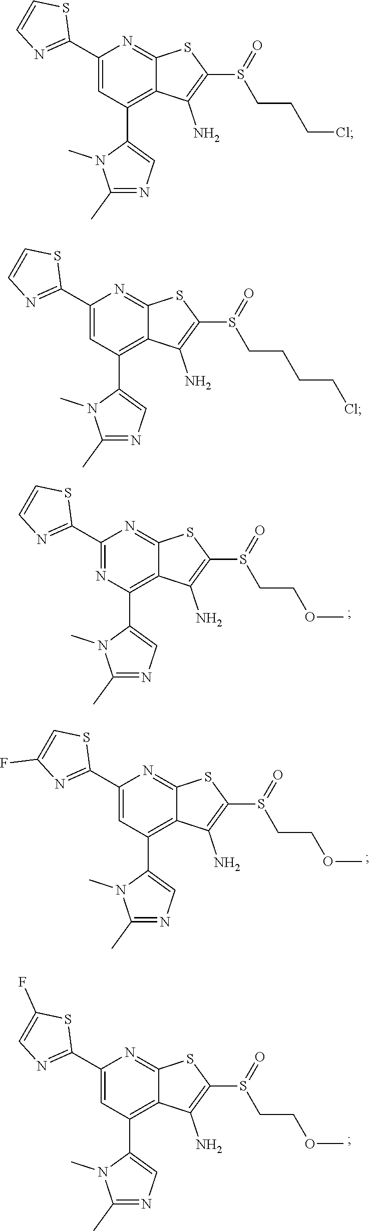

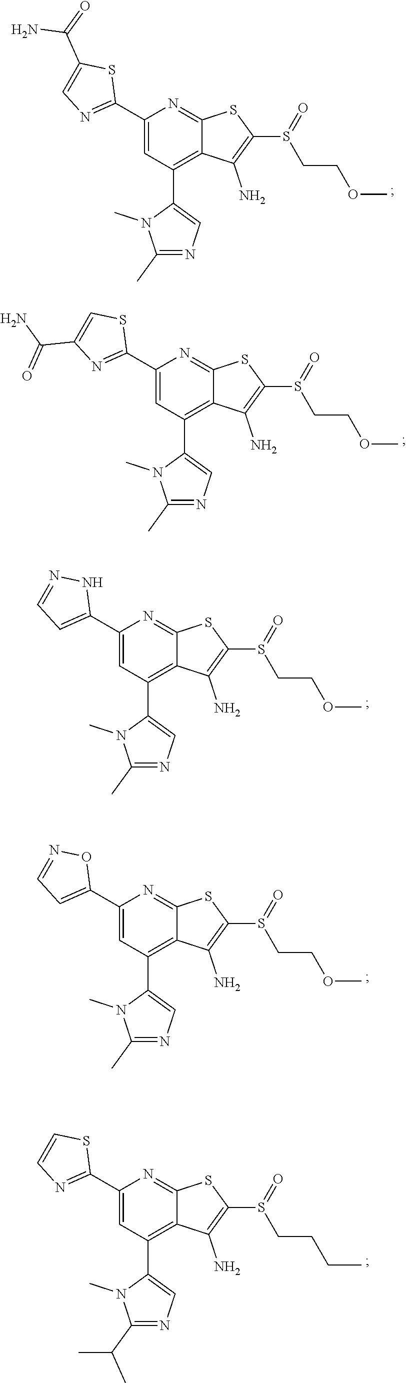

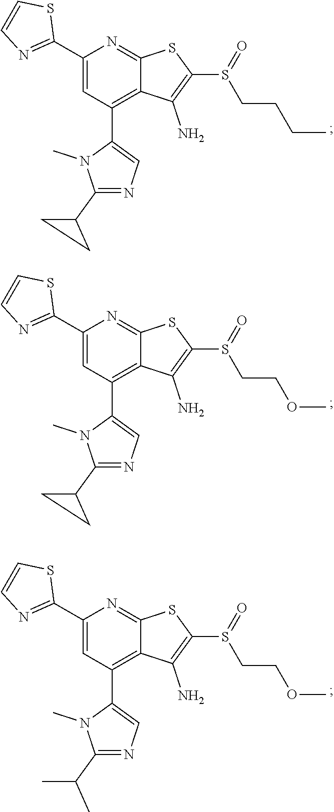 [SMILES: CCCCS(=O)c1sc2nc(-c3nccs3)cc(-c3cnc(C4CC4)n3C)c2c1N ; COCCS(=O)c1sc2nc(-c3nccs3)cc(-c3cnc(C4CC4)n3C)c2c1N ; COCCS(=O)c1sc2nc(-c3nccs3)cc(-c3cnc(C(C)C)n3C)c2c1N]

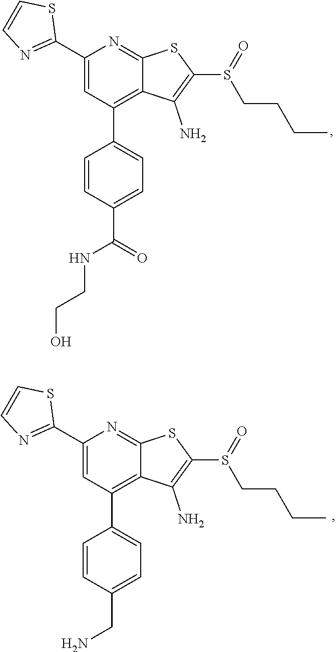 [SMILES: CCCCS(=O)c1sc2nc(-c3nccs3)cc(-c3ccc(C(=O)NCCO)cc3)c2c1N ; CCCCS(=O)c1sc2nc(-c3nccs3)cc(-c3ccc(CN)cc3)c2c1N]

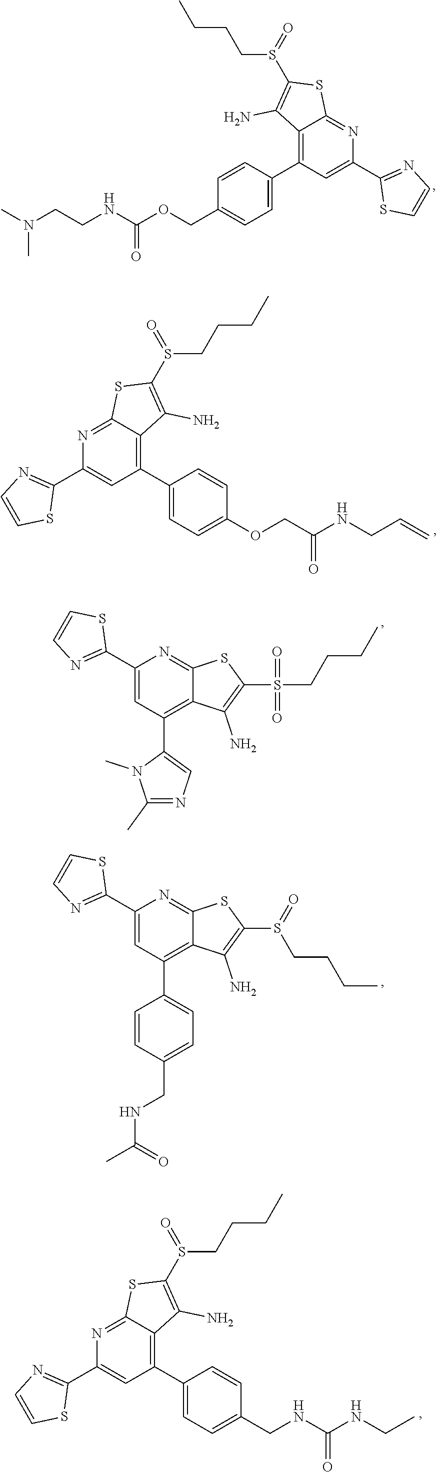

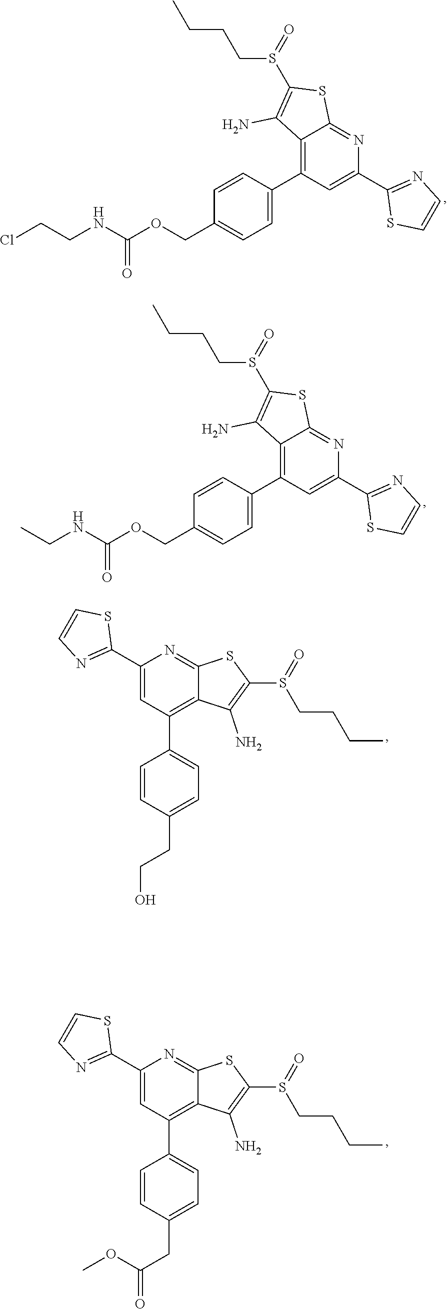

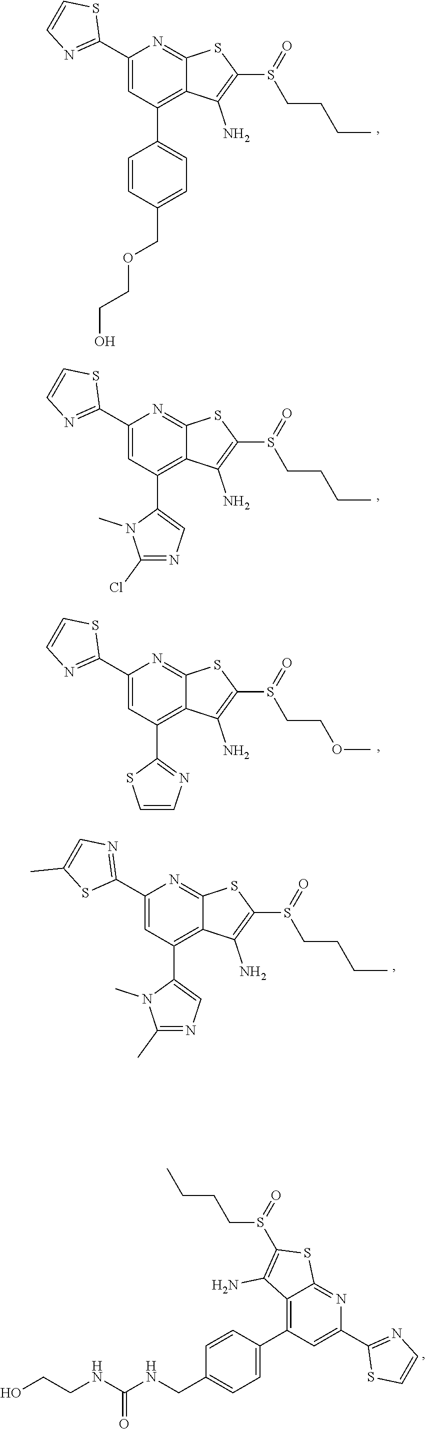

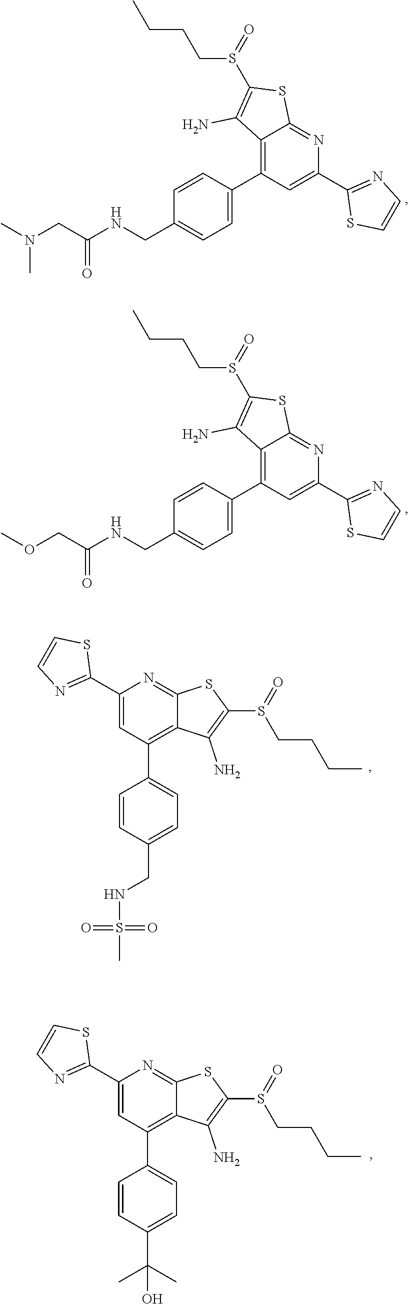 [SMILES: CCCCS(=O)c1sc2nc(-c3nccs3)cc(-c3ccc(CNC(=O)CN(C)C)cc3)c2c1N ; CCCCS(=O)c1sc2nc(-c3nccs3)cc(-c3ccc(CNC(=O)COC)cc3)c2c1N ; CCCCS(=O)c1sc2nc(-c3nccs3)cc(-c3ccc(CNS(C)(=O)=O)cc3)c2c1N ; CCCCS(=O)c1sc2nc(-c3nccs3)cc(-c3ccc(C(C)(C)O)cc3)c2c1N]

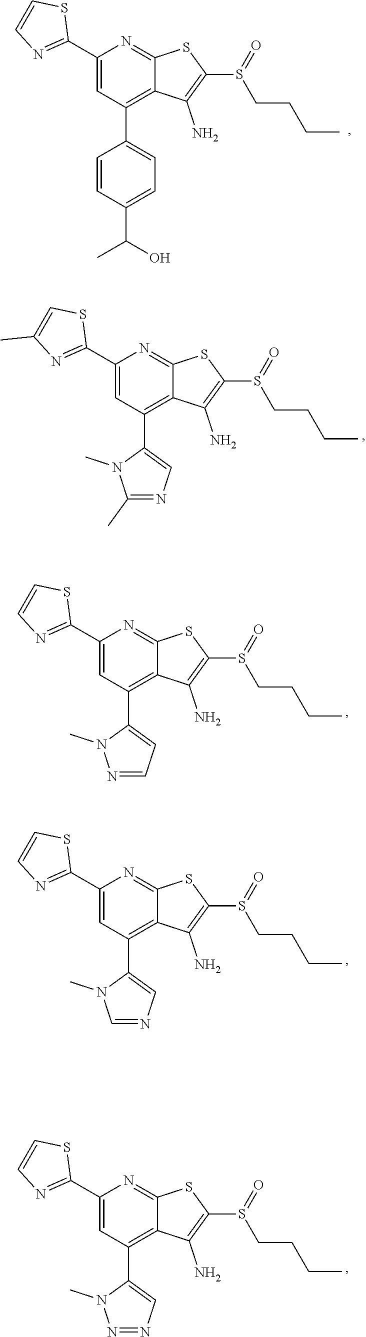

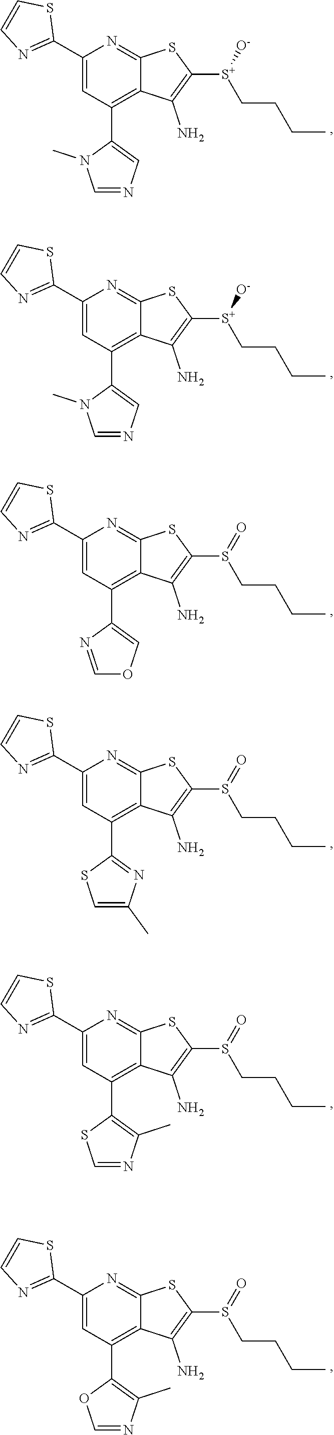 [SMILES: CCCC[S@@+]([O-])c1sc2nc(-c3nccs3)cc(-c3cncn3C)c2c1N ; CCCC[S@+]([O-])c1sc2nc(-c3nccs3)cc(-c3cncn3C)c2c1N ; CCCCS(=O)c1sc2nc(-c3nccs3)cc(-c3cocn3)c2c1N ; CCCCS(=O)c1sc2nc(-c3nccs3)cc(-c3nc(C)cs3)c2c1N ; CCCCS(=O)c1sc2nc(-c3nccs3)cc(-c3scnc3C)c2c1N ; CCCCS(=O)c1sc2nc(-c3nccs3)cc(-c3ocnc3C)c2c1N]

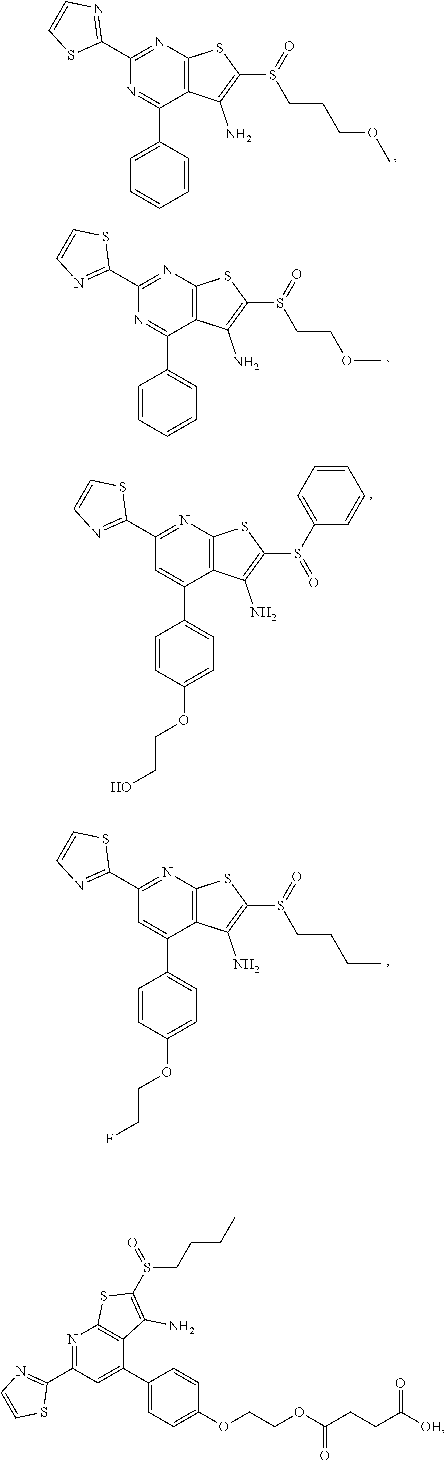 [SMILES: COCCCS(=O)c1sc2nc(-c3nccs3)nc(-c3ccccc3)c2c1N ; COCCS(=O)c1sc2nc(-c3nccs3)nc(-c3ccccc3)c2c1N ; Nc1c(S(=O)c2ccccc2)sc2nc(-c3nccs3)cc(-c3ccc(OCCO)cc3)c12 ; CCCCS(=O)c1sc2nc(-c3nccs3)cc(-c3ccc(OCCF)cc3)c2c1N ; CCCCS(=O)c1sc2nc(-c3nccs3)cc(-c3ccc(OCCOC(=O)CCC(=O)O)cc3)c2c1N]

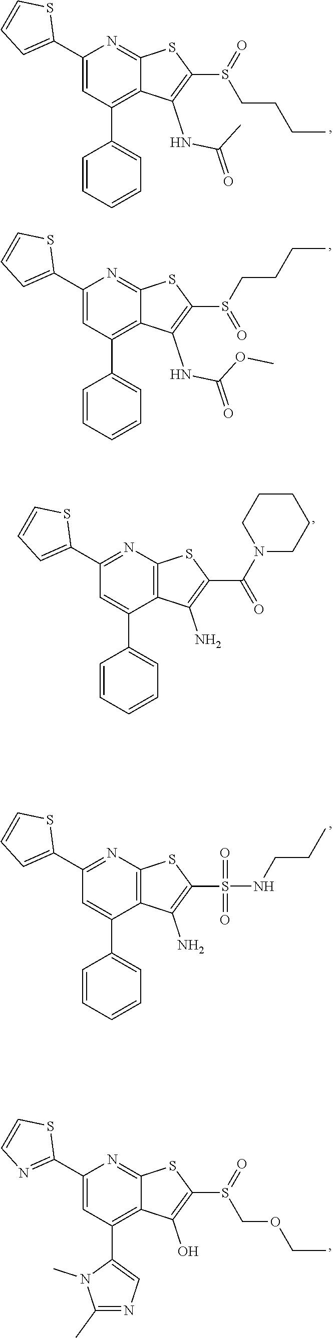 [SMILES: CCCCS(=O)c1sc2nc(-c3cccs3)cc(-c3ccccc3)c2c1NC(C)=O ; CCCCS(=O)c1sc2nc(-c3cccs3)cc(-c3ccccc3)c2c1NC(=O)OC ; Nc1c(C(=O)N2CCCCC2)sc2nc(-c3cccs3)cc(-c3ccccc3)c12 ; CCCNS(=O)(=O)c1sc2nc(-c3cccs3)cc(-c3ccccc3)c2c1N ; CCOCS(=O)c1sc2nc(-c3nccs3)cc(-c3cnc(C)n3C)c2c1O]

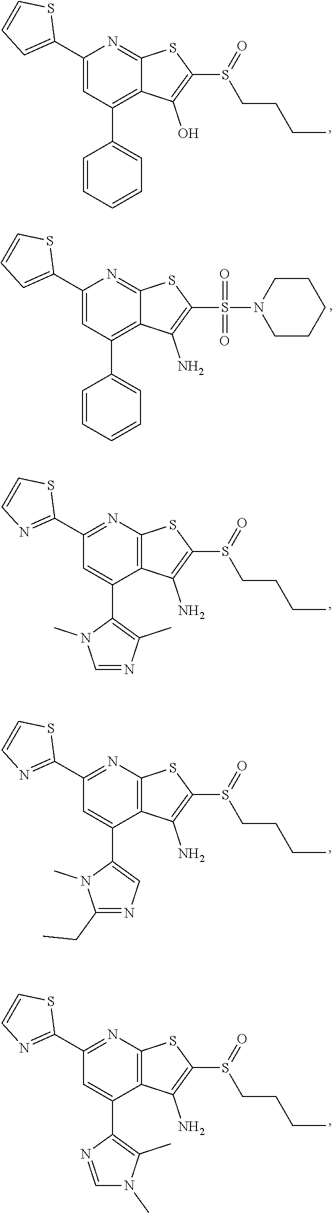 [SMILES: CCCCS(=O)c1sc2nc(-c3cccs3)cc(-c3ccccc3)c2c1O ; Nc1c(S(=O)(=O)N2CCCCC2)sc2nc(-c3cccs3)cc(-c3ccccc3)c12 ; CCCCS(=O)c1sc2nc(-c3nccs3)cc(-c3c(C)ncn3C)c2c1N ; CCCCS(=O)c1sc2nc(-c3nccs3)cc(-c3cnc(CC)n3C)c2c1N ; CCCCS(=O)c1sc2nc(-c3nccs3)cc(-c3ncn(C)c3C)c2c1N]

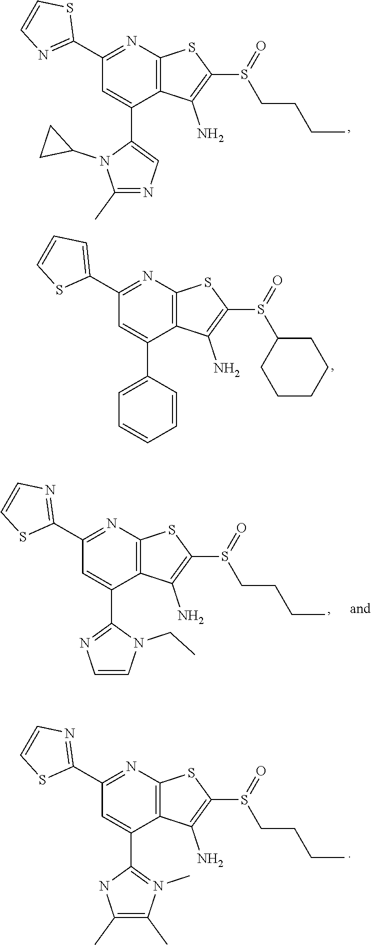

In some embodiments and without being limited by theory, Applicants unexpectedly and surprisingly discovered that carbocyclyl groups at the R¹ position increase the half-life of the compound. Half-life is an important parameter of a therapeutic agent because it determines that duration of action of the agent—the longer the half-life, the longer the agent is able to exert a therapeutic effect in the subject. In some embodiments, 3- to 6-membered cycloalkyls, particularly 3- to 5-membered cycloalkyls such as cyclobutyl, were observed to have desirable half-lives.

In other embodiments, the compound has the structure of formula (II):

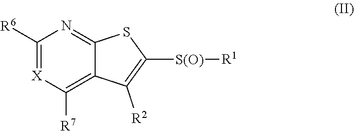

or a pharmaceutically acceptable salt, tautomer, or solvate thereof, wherein:
R¹ is 3- to 6-membered cycloalkyl, —(C₁-C₆ alkylene)-(3- to 6-membered cycloalkyl), 3- to 6-membered heterocyclyl, or —(C₁-C₆ alkylene)-(3- to 6-membered heterocyclyl);
R² is —NH₂, CN, or —NHC(O)alkyl;
R⁶ is heterocyclyl or heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R′)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)t, or N;
X is N or CH;
m is 0, 1, or 2; and
t is 0, 1, or 2.

In some embodiments, R¹ is 3- to 5-membered cycloalkyl or —(C₁-C₆ alkylene)-(3- to 5-membered cycloalkyl).

In other embodiments, R¹ is cyclobutyl.

In still other embodiments, R¹ is a bicyclic 4- to 6-membered cycloalkyl.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In other embodiments, R⁷ is C₁-C₃ alkyl, C₁-C₃ haloalkyl, 3-membered cycloalkyl, phenyl, 4-membered heterocyclyl, or 5- to 6-membered heteroaryl, each of which is optionally substituted with one or more R⁴. In other embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, isopropyl, cyclopropyl, phenyl, pyridyl, pyrazole, or triazole, each of which is optionally substituted with one or more R⁴. In other embodiments, R⁷ is —CF₃, cyclopropyl, phenyl, pyridyl, pyrazole, or triazole, each of which is optionally substituted with one or more R⁴.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments and without being limited by theory, Applicants surprisingly and unexpectedly discovered that substituents at the R⁷ position could be modified to improve hERG activity, including hERG inhibition (IC₅₀), blockade, and efflux ratio. For example, in some embodiments, certain 6- to 10-membered aryls (e.g., optionally substituted phenyl) and 5- to 10-membered heteroaryls (e.g., optionally substituted pyridyls, pyrazoles, and triazoles) were observed to have beneficial hERG properties. In some embodiments, certain C₁-C₆ haloalkyls (e.g., —CF₃) exhibited improved hERG inhibition (IC50), while also improving half-life and solubility.

In other embodiments, the compound has the structure of formula (III):

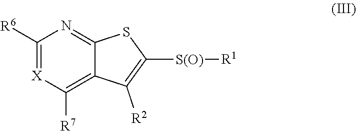

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is heterocyclyl or heteroaryl, each of which is optionally substituted with one or more R³;
R⁷ is C₁-C₆ haloalkyl, aryl or heteroaryl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁴ is halogen, alkyl, or alkoxy;
X is N or CH; and
m is 0, 1, or 2.

In some embodiments, R⁷ is —CF₃, pyridyl, pyrazole, phenyl, or triazole, each of which is optionally substituted with R⁴.

In other embodiments, R⁷ is —CF₃, pyridyl, fluorophenyl, or a triazole optionally substituted with halogen or methyl.

In other embodiments, R⁷ is —CF₃,

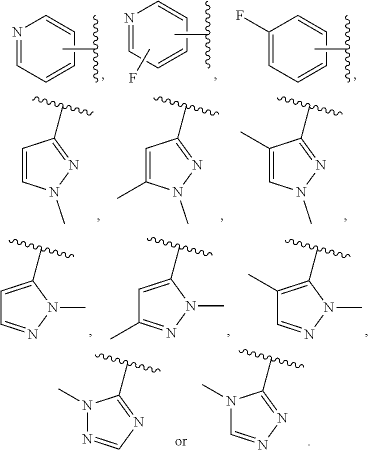

In still other embodiments, R⁷ is —CF₃.

In still other embodiments, R⁷ is

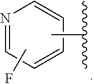

In some embodiments, R⁷ is

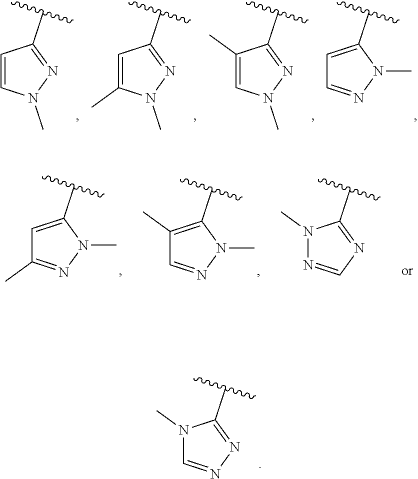

In some embodiments, R⁷ is

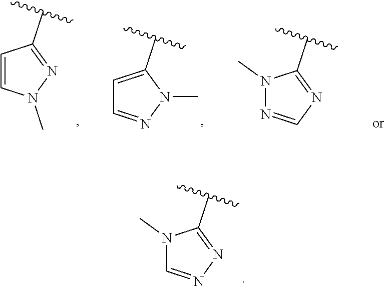

In other embodiments, R⁶ is 8- to 10-membered bicyclic heteroaryl optionally substituted with one or more R³.

In some embodiments and without being limited by theory, Applicants surprisingly and unexpectedly discovered that 3- to 6-membered cycloalkyls at the R⁷ position can improve solubility while maintaining PDGH activity.

In other embodiments, the compound has the structure of formula (IV):

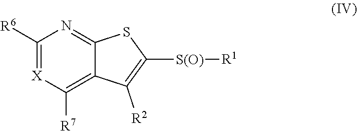

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is optionally substituted with one or more R³;
R⁷ is 3- to 6-membered cycloalkyl, optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is halogen, —CN, —NH₂, —OH, or C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, -alkylene-COOH, —C(O)O-alkyl, or —S(O)ₘ-alkyl;
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R⁷ is cyclopropyl.

In other embodiments, R¹ is 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In some embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In some embodiments and without being limited by theory, Applicants surprisingly and unexpectedly discovered that the R⁶ position can be substituted with certain R³ groups to improve solubility and activity.

In other embodiments, the compound has the structure of formula (V):

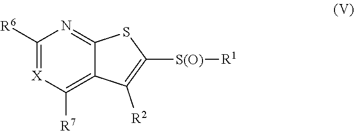

or a pharmaceutically acceptable salt, tautomer, or solvate thereof, wherein:
R¹ is cycloalkyl, -alkylene-cycloalkyl, -alkylene-alkoxy, heterocyclyl, or -alkylene-heterocyclyl;
R² is —NH₂, CN, or —NHC(O)alkyl;
R⁶ is heterocyclyl or heteroaryl, each of which is substituted with one or more R³;
R⁷ is haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl);
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently H, C₁-C₆ alkyl, —(C₁-C₆ alkylene)-OH optionally substituted with —OH, -alkylene-NH₂, -alkylene-N(R⁹)₂, -alkylene-O-alkylene-OH, -alkylene-O-alkylene-NH₂, —C(O)(C₁-C₆ alkyl), —C(O)O(C₁-C₆ alkyl), alkylene-COOH, or —S(O)ₘ(C₁-C₆ alkyl);
or alternative, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH;
m is 0, 1, or 2.

In some embodiments, R³ is —O—(C₁-C₆ alkylene)-N(R⁵)₂, —N(R⁵)₂ or —N(R⁵)(C₁-C₆ alkylene-OH).

In other embodiments, R⁵ is H, C₁-C₆ alkyl, —(C₁-C₆ alkylene)—OH, or —S(O)₂(C₁-C₃ alkyl).

In some embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In other embodiments, R³ is —NHCH₂CH₂OH or —N(CH₃)CH₂CH₂OH.

In still other embodiments, R³ is —NHCH₂CH₂OH.

In some embodiments, R⁶ is 5- to 6-membered heterocyclyl or 5- to 10-membered heteroaryl, each of which is optionally substituted with one or more R³.

In other embodiments, R⁶ is 5- to 6-membered heteroaryl optionally substituted with one or more R³.

In some embodiments, R⁶ is furan, thiophene, pyrrole, thiazole, isothiazole, oxazole, isooxazole, pyrazole, imidazole, triazole, pyridine, pyrimidine, pyridazine, or pyrazine, each optionally substituted with one or more R³.

In other embodiments, R⁶ is thiazole, imidazole, oxazole, pyridine, or pyrimidine.

In some embodiments, R⁶ is 8- to 10-membered bicyclic heteroaryl optionally substituted with one or more R³.

In other embodiments, R⁶ is 5- to 6-membered heterocyclyl, optionally substituted with one or more R³, selected from morpholine, pyridine-one, or piperidine.

In some embodiments, R⁷ is C₁-C₃ haloalkyl, 3-membered cycloalkyl, phenyl, 4-membered heterocyclyl, or 5- to 6-membered heteroaryl, each of which is optionally substituted with one or more R⁴.

In other embodiments, R⁷ is —CF₃, cyclopropyl, phenyl, pyrazole, pyridyl, or triazole, each of which is optionally substituted with one or more R⁴.

In some embodiments, the compound has the structure of formula (VI):

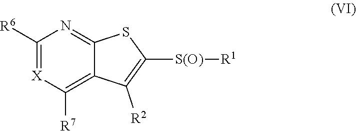

or a pharmaceutically acceptable salt, tautomer, or solvate thereof, wherein:
R¹ is cycloalkyl, alkylene-cycloalkyl, alkylene-alkoxy, heterocyclyl, or alkylene-heterocyclyl;
R² is —NH₂, CN, or —NHC(O)alkyl;
R⁶ is fused bicyclic heterocyclyl or fused bicyclic heteroaryl, each of which is optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)— alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-O-alkylene-OH, -alkylene-O-alkylene-NH₂, —C(O)-alkyl, —C(O)O-alkyl, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
X is N or CH; and
m is 0, 1, or 2.

In some embodiments, R⁶ is 8- to 10-membered fused bicyclic heteroaryl, each of which is optionally substituted with one or more R.

In some embodiments, R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

In other embodiments, R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴. In other embodiments, R⁷ is C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

In still other embodiments, R⁷ is C₁-C₃ alkyl, C₁-C₆ haloalkyl, 3- to 6-membered cycloalkyl, phenyl, 3- to 6-membered heterocyclyl, or 5- to 6-membered heteroaryl, each of which is optionally substituted with one or more R⁴.

In other embodiments, R⁷ is C₁-C₃ alkyl, C₁-C₆ haloalkyl, 3- to 6-membered cycloalkyl, phenyl, pyrazole, pyridyl, or triazole, each of which is optionally substituted with one or more R⁴. In other embodiments, R⁷ is C₁-C₆ haloalkyl, 3- to 6-membered cycloalkyl, phenyl, pyrazole, pyridyl, or triazole, each of which is optionally substituted with one or more R⁴.

In other embodiments, R⁷ is —CF₃, isopropyl, cyclopropyl, phenyl, pyridyl, or triazole, each of which is optionally substituted with one or more R⁴. In other embodiments, R⁷ is —CF₃, cyclopropyl, phenyl, pyridyl, or triazole, each of which is optionally substituted with one or more R⁴.

In some embodiments, R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

In other embodiments, R¹ is cyclopropyl, cyclobutyl, cyclopentyl, cyclohexyl, —(CH₂)ₚ-cyclopropyl, —(CH₂)ₚ-cyclobutyl, —(CH₂)ₚ-cyclopentyl, or —(CH₂)ₚ-cyclohexyl; wherein p is 1, 2, or 3.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In some embodiments, R³ is —NHCH₂CH₂OH or —N(CH₃)CH₂CH₂OH.

In other embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, the compound has the structure of formula (VII):

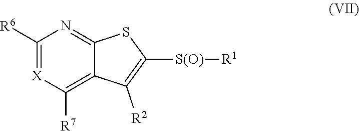

or a pharmaceutically acceptable salt, tautomer, or solvate thereof, wherein:
R¹ is cyclobutyl or —(C₁-C₄ alkylene)—(C₁-C₃ alkoxy);
R² is —NH₂, CN, or —NHC(O)alkyl;
R⁶ is heterocyclyl or heteroaryl, each of which is optionally substituted with one or more R³.
R⁷ is —CF₃, isopropyl,

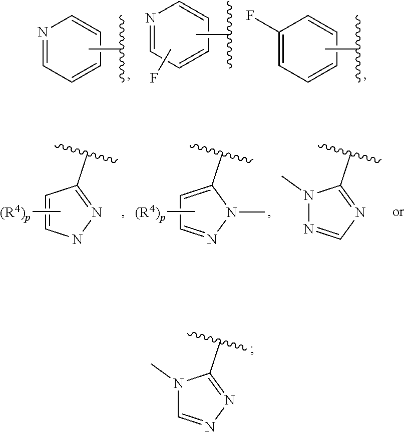

R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is C₁-C₃ alkyl;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
R¹⁰ is —OH, halogen, alkyl, or alkoxy;
X is N or CH;
m is 0, 1, or 2;
p is 0 or 1; and
t is 0, 1, or 2.

In still other embodiments, R² is —NH₂.

In some embodiments, R⁶ is 5- to 6-membered heterocyclycl or 5- to 10-membered heteroaryl, each of which is optionally substituted with one or more R³.

In other embodiments, R⁶ is 5- to 6-membered heteroaryl optionally substituted with one or more R³.

In still other embodiments, R⁶ is 8- to 10-membered bicyclic heteroaryl optionally substituted with one or more R³.

In some embodiments, R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

In other embodiments, R³ is —(C₁-C₃ alkyl)OH, —NH₂, —N(C₁-C₃ alkyl)₂, —NHCH₂CH₂OH, —N(C₁-C₃ alkyl)CH₂CH₂OH, N(CH₂CH₂OH)₂, —NHCH₂CH(CH₂OH)₂, —N(C₁-C₃ alkyl)CH₂CH(CH₂OH)₂, —NHCH₂CH₂OCH₂CH₂OH, —NHCH₂CH₂OCH₂CH₂NH₂, —NHCH₂CH₂NH₂, —N(C₁-C₃ alkyl)CH₂CH₂NH₂, —NHCH₂CH₂NH(C₁-C₃ alkyl), —NHCH₂CH₂N(C₁-C₃ alkyl)₂, —N(C₁-C₃ alkyl)CH₂CH₂NH(C₁-C₃ alkyl), —N(C₁-C₃ alkyl)CH₂CH₂N(C₁-C₃ alkyl)₂, —NHSO₂CH₃, —N(C₁-C₃ alkyl)SO₂CH₃, —OCH₂CH₂OH, —OCH₂CH₂NH₂, —OCH₂CH₂NH(C₁-C₃ alkyl), or —OCH₂CH₂N(C₁-C₃ alkyl)₂.

In still other embodiments, R³ is —NHCH₂CH₂OH or —N(CH₃)CH₂CH₂OH.

In some embodiments, R⁴ is halogen, —CN, —N(R⁵)₂, —OH, —O—(C₁-C₆ alkylene)-OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

In some embodiments, n is 1.

In some embodiments, the compound has the structure of formula (VIII):

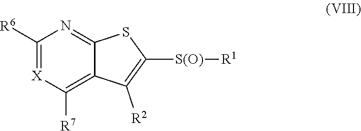

or a pharmaceutically acceptable salt, tautomer, or solvate thereof, wherein:
R¹ is cyclobutyl or —(C₁-C₄ alkylene)—(C₁-C₃ alkoxy);
R² is —NH₂, CN, or —NHC(O)alkyl; R⁶ is

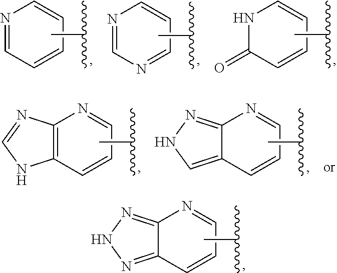

each of which is optionally substituted with one or more R³;
R⁷ is —CF₃, isopropyl, cyclopropyl, cyclobutyl,

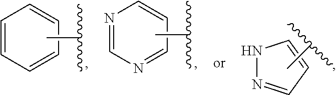

each of which is optionally substituted with one or more R⁴;
R³ is —NH₂, —NH(C₁-C₃ alkyl), —NH(C₁-C₄ alkylene)—OH, or C₁-C₃ alkyl;
R⁴ is C₁-C₃ alkyl; and
X is N or CH.

In some embodiments of Formula (VIII), R¹ is cyclobutyl. In some embodiments of Formula (VIII), R¹—(C₁-C₄ alkylene)—(C₁-C₃ alkoxy). In some embodiments of Formula (VIII), the R¹—(C₁-C₄ alkylene)—(C₁-C₃ alkoxy) is —(C₂-C₃ alkylene)-(C₁ alkoxy).

In some embodiments of Formula (VIII), R² is —NH₂.

In some embodiments of Formula (VIII), R⁶ is

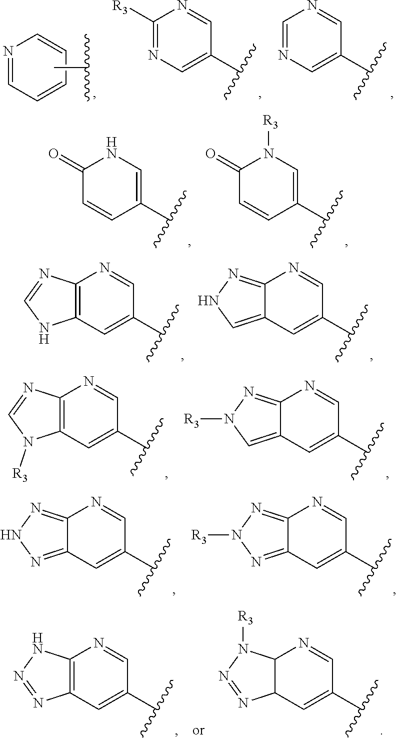

In some embodiments of Formula (VIII), R³ is —NH₂. In some embodiments of Formula (VIII), R³ is —NH(C₁-C₃ alkyl). In some embodiments of Formula (VIII), R³ is —NH(C₁-C₄ alkylene)—OH (e.g., —NH(C₂-C₄ alkylene)—OH). In some embodiments of Formula (VIII), R³ is C₁-C₃ alkyl (e.g., methyl or ethyl).

In some embodiments, of Formula (VIII), R⁷ is —CF₃, isopropyl, cyclopropyl, or cyclobutyl. In some embodiments, of Formula (VIII), R⁷ is isopropyl. In some embodiments of Formula (VII), R⁷ is

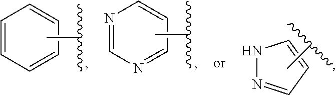

each of which is optionally substituted with one or more R⁴. In some embodiments, each R⁴ is independently selected from methyl or ethyl.

In some embodiments of Formula (VIII), X is —CH.

In some embodiments, the compounds of Formula (VIII) are provided in Table 2.

In one embodiment, the compounds of Formula (VIII) excludes:

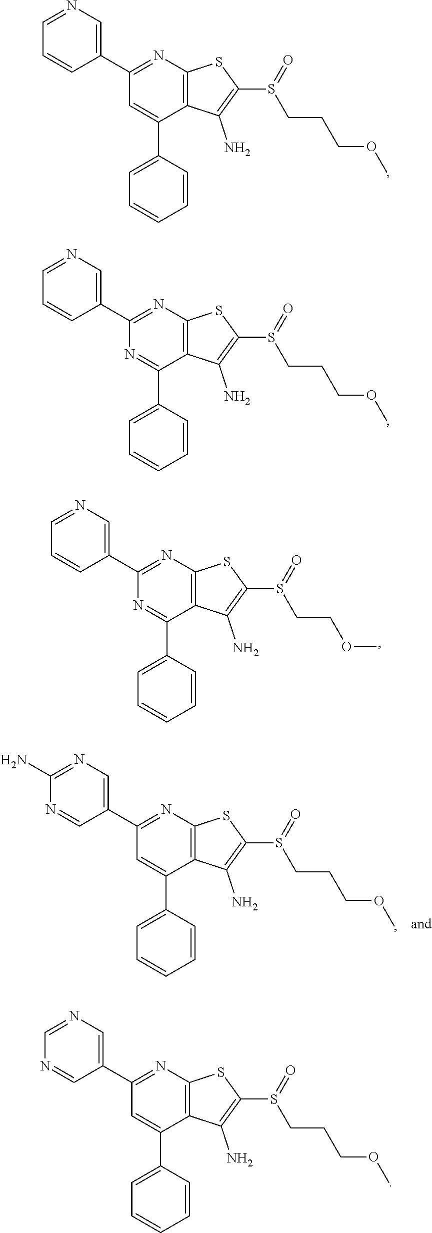

In some embodiments, the 15-PGDH inhibitor of the present disclosure inhibit colon 15-PGDH activity in a range of from about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. Colon 15-PGDH inhibition can be measured (e.g., with the assay described in this present examples) using an appropriate dose of the compounds of the present disclosure, at 30 min, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 15 hours, 20 hours, 24 hours, 48 hours, 72 hours, or more hours after administration, including all times between these values. In some embodiments, colon 15-PGDH inhibition is measured at 30 minutes after administration. In particular embodiments, colon 15-PGDH inhibition is measured at 4 hours. In some embodiments, the appropriate dose is 1 2, 3, 4, 5, 6, 7, 8, 9, 0 15, 20, 30, 40, 50, or more mg/kg, including all values and ranges in between these values.

In one embodiment, lung, liver, intestines, the skin, heart (or any other organ disclosed herein) 15-PGDH inhibition can be measured using an appropriate dose of the compounds of the present disclosure, at 30 min, 1 hour, 1.5 hours, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 15 hours, 20 hours, 24 hours, 48 hours, 72 hours, or more, including all times and ranges in between these values. In some embodiments, lung 15-PGDH inhibition is measured at 30 minutes. In particular embodiments, lung 15-PGDH inhibition is measured at 4 hours. In some embodiments, the appropriate dose is 1 2, 3, 4, 5, 6, 7, 8, 9, 0 15, 20, 30, 40, 50, or more mg/kg, including all values and ranges in between these values. In some embodiments, the 15-PGDH inhibitor of the present disclosure inhibit lung 15-PGDH activity in a range of from about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein.

In some embodiments, the 15-PGDH inhibitor of the present disclosure (e.g., having formula I-VIII), is administered at 10 mg/kg in a mammal and inhibits colon 15-PGDH activity at 30 minutes in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In some embodiments, the compounds of the present disclosure, when administered at 10 mg/kg in a mammal, inhibit colon 15-PGDH activity at 30 minutes in a range of about 65% to 100% (e.g., about 65%, about 70%, about 75%, about 80%, about 85%, about 90%, about 95%, or about 100%), and any subranges therein. In some embodiments, the compounds of the present disclosure when administered at 10 mg/kg in a mammal can inhibit colon 15-PGDH activity at 30 minutes in a range of about 70% to 100% (e.g., about 70%, about 75%, about 80%, about 85%, about 90%, about 95%, or about 100%), and any subranges therein. In some embodiments, the compounds of the present disclosure, when administered at 10 mg/kg in a mammal, inhibit colon 15-PGDH activity at 30 minutes in a range of about 80% to 100%, and any subranges therein. In some embodiments, the compounds of the present disclosure, when administered at 10 mg/kg in a mammal, inhibit colon 15-PGDH activity at 30 minutes in a range of about 90% to 100%, and any subranges therein. In some embodiments, the compounds of the present disclosure, when administered at 10 mg/kg in a mammal, inhibit colon 15-PGDH activity at 30 minutes in a range of about 95% to 100%, and any subranges therein. This is a unexpected and surprising result, as previously synthesized 15-PGDH inhibitors, such as Reference Compound C (Example B), when given orally at 10 mpk was not sufficiently potent to inhibit colon 15-PGDH activity at 30 minutes. In fact, Reference Compound C actually increased activity colon 15-PGDH activity, as indicated by the -55.2% inhibition.

In some embodiments, the 15-PGDH inhibitor of the present disclosure (e.g., having formula I-VIII), is administered at 10 mg/kg in a mammal and inhibits colon 15-PGDH activity at 4 hours in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In some embodiments, the compounds of the present disclosure, when administered at 10 mg/kg in a mammal, inhibit colon 15-PGDH activity at 4 hours in a range of about 65% to 100% (e.g., about 65%, about 70%, about 75%, about 80%, about 85%, about 90%, about 95%, or about 100%), and any subranges therein. In some embodiments, the compounds of the present disclosure when administered at 10 mg/kg in a mammal can inhibit colon 15-PGDH activity at 4 hours in a range of about 70% to 100% (e.g., about 70%, about 75%, about 80%, about 85%, about 90%, about 95%, or about 100%), and any subranges therein. In some embodiments, the compounds of the present disclosure, when administered at 10 mg/kg in a mammal, inhibit colon 15-PGDH activity at 4 hours in a range of about 80% to 100%, and any subranges therein. In some embodiments, the compounds of the present disclosure, when administered at 10 mg/kg in a mammal, inhibit colon 15-PGDH activity at 4 hours in a range of about 80% to 98%, and any subranges therein.

In some embodiments, the 15-PGDH inhibitor of the present disclosure (e.g., having formula I-VIII), is administered at 10 mg/kg in a mammal and inhibits lung, liver, intestines, the skin, heart (or any other organ disclosed herein) 15-PGDH activity at 30 minutes in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In some embodiments, the compounds of the present disclosure, when administered at 10 mg/kg in a mammal, inhibit lung 15-PGDH activity at 30 minutes in a range of about 65% to 100% (e.g., about 65%, about 70%, about 75%, about 80%, about 85%, about 90%, about 95%, or about 100%), and any subranges therein. In some embodiments, the compounds of the present disclosure when administered at 10 mg/kg in a mammal can inhibit lung 15-PGDH activity at 30 minutes in a range of about 70% to 100% (e.g., about 70%, about 75%, about 80%, about 85%, about 90%, about 95%, or about 100%), and any subranges therein. In some embodiments, the compounds of the present disclosure, when administered at 10 mg/kg in a mammal, inhibit lung 15-PGDH activity at 30 minutes in a range of about 80% to 100%, and any subranges therein.

In some embodiments, the 15-PGDH inhibitor of the present disclosure (e.g., having formula I-VIII), is administered at 10 mg/kg in a mammal and inhibits lung, liver, intestines, the skin, heart (or any other organ disclosed herein) 15-PGDH activity at 4 hours in a range of about 25% to 100%, e.g., about 25%, about 30%, about 35%, about 40%, about 45%, about 50%, about 55%, about 60%, about 65%, about 70%, about 75%, about 80%, about 85%, about 90%, about 95%, or about 100%, and any subranges therein. In some embodiments, the compounds of the present disclosure, when administered at 10 mg/kg in a mammal, inhibit lung 15-PGDH activity at 4 hours in a range of about 65% to 100% (e.g., about 65%, about 70%, about 75%, about 80%, about 85%, about 90%, about 95%, or about 100%), and any subranges therein. In some embodiments, the compounds of the present disclosure when administered at 10 mg/kg in a mammal can inhibit lung 15-PGDH activity at 4 hours in a range of about 70% to 100% (e.g., about 70%, about 75%, about 80%, about 85%, about 90%, about 95%, or about 100%), and any subranges therein. In some embodiments, the compounds of the present disclosure, when administered at 10 mg/kg in a mammal, inhibit lung 15-PGDH activity at 4 hours in a range of about 80% to 100%, and any subranges therein.

In some embodiments, the 15-PGDH inhibitors of the present disclosure (e.g., formula I-VIII) have a human microsome stability T_{1/2} of greater than 50 minutes. In some embodiments, the compounds of the present disclosure has a human microsome stability T_{1/2} of greater than 60 minutes. In some embodiments, the 15-PGDH inhibitors of the present disclosure have a human microsome stability T_{1/2} of greater than 70 minutes. In some embodiments, the 15-PGDH inhibitors of the present disclosure have a human microsome stability T_{1/2} of greater than 80 minutes. In some embodiments, the 15-PGDH inhibitors of the present disclosure have a human microsome stability T_{1/2} of greater than 90 minutes. In some embodiments, the 15-PGDH inhibitors of the present disclosure have a human microsome stability T_{1/2} of greater than 100 minutes. In some embodiments, the compounds of the present disclosure has a human microsome stability T_{1/2} of greater than 110 minutes. In some embodiments, the 15-PGDH inhibitors of the present disclosure have a human microsome stability T_{1/2} of greater than 120 minutes. In some embodiments, the 15-PGDH inhibitors of the present disclosure have a human microsome stability T_{1/2} of greater than 130 minutes. In some embodiments, the 15-PGDH inhibitors of the present disclosure have a human microsome stability T_{1/2} of greater than 145 minutes. In some embodiments, the 15-PGDH inhibitors of the present disclosure have a human microsome stability T_{1/2} ranging from 65 to at least 145 (e.g., 65, 70, 80, 90, 100, 110, 120, 130, 140, 150, 160, 170, 180, 190, 200, or more, including all values and ranges therebetween).

In some embodiments, the 15-PGDH inhibitors of the present disclosure (e.g., formula I-VIII) have better human microsome stability compared to previously disclosed 15-PGDH inhibitors. See WO 2013/158649, WO 2015/065716, WO 2016/144958, WO 2016/168472, WO 2018/017582, WO 2018/102552, WO 2018/145080, WO 2018/187810, and WO 2018/218251, the disclosures of each are hereby incorporated by reference in their entireties for all purposes. In some embodiments, the 15-PGDH inhibitors of the present disclosure have a human microsome stability T_{1/2} which is at least 15 minutes longer than previously disclosed 15-PGDH inhibitors. In some embodiments, the 15-PGDH inhibitors of the present disclosure have a human microsome stability T_{1/2} which is at least 25 minutes longer than previously disclosed 15-PGDH inhibitors. In some embodiments, the 15-PGDH inhibitors of the present disclosure have a human microsome stability T_{1/2} which is at least 35 minutes longer than previously disclosed 15-PGDH inhibitors. In some embodiments, the 15-PGDH inhibitors of the present disclosure have a human microsome stability T_{1/2} which is at least 45 minutes longer than previously disclosed 15-PGDH inhibitors. In some embodiments, the 15-PGDH inhibitors of the present disclosure have a human microsome stability T_{1/2} which is at least 55 minutes longer than previously disclosed 15-PGDH inhibitors. In some embodiments, the 15-PGDH inhibitors of the present disclosure have a human microsome stability T_{1/2} which is at least 65 minutes longer than previously disclosed 15-PGDH inhibitors. In some embodiments, the 15-PGDH inhibitors of the present disclosure have a human microsome stability T_{1/2} which is at least 75 minutes longer than previously disclosed 15-PGDH inhibitors. In some embodiments, the 15-PGDH inhibitors of the present disclosure have a human microsome stability T_{1/2} which is at least 85 minutes longer than previously disclosed 15-PGDH inhibitors. In some embodiments, the 15-PGDH inhibitors of the present disclosure have human microsome stability T_{1/2} which is at least 95 minutes longer than previously disclosed 15-PGDH inhibitors. In some embodiments, the 15-PGDH inhibitors of the present disclosure have a human microsome stability T_{1/2} which is at least 100 minutes longer than previously disclosed 15-PGDH inhibitors. In some embodiments, the 15-PGDH inhibitors of the present disclosure have a human microsome stability T_{1/2} which is at least 110 minutes longer than previously disclosed 15-PGDH inhibitors. In some embodiments, the 15-PGDH inhibitors of the present disclosure have a human microsome stability T_{1/2} which is at least 120 minutes longer than previously disclosed 15-PGDH inhibitors. In some embodiments, the 15-PGDH inhibitors of the present disclosure have a human microsome stability T_{1/2} ranging that is from 15 minutes to about 120 minutes longer than the microsome stability T_{1/2} of previously disclosed 15-PGDH inhibitors.

In some embodiments, the 15-PGDH inhibitors of the present disclosure (e.g., formula I-VIII) have a human microsome stability T_{1/2} which is at least 40 minutes longer than Reference Compound C (Example B). In some embodiments, the 15-PGDH inhibitors of the present disclosure have a human microsome stability T_{1/2} which is at least 70 minutes longer than Reference Compound C. In some embodiments, the 15-PGDH inhibitors of the present disclosure have a human microsome stability T_{1/2} which is at least 80 minutes longer than Reference Compound C. In some embodiments, the 15-PGDH inhibitors of the present disclosure have a human microsome stability T_{1/2} which is at least 90 minutes longer than Reference Compound C. In some embodiments, the 15-PGDH inhibitors of the present disclosure have a human microsome stability T_{1/2} which is at least 100 minutes longer than Reference Compound C. In some embodiments, the 15-PGDH inhibitors of the present disclosure have a human microsome stability T_{1/2} which is at least 110 minutes longer than Reference Compound C. In some embodiments, the 15-PGDH inhibitors of the present disclosure have a human microsome stability T_{1/2} which is at least 120 minutes longer than Reference Compound C. In some embodiments, the 15-PGDH inhibitors of the present disclosure have a human microsome stability T_{1/2} ranging that is from 70 minutes to about 120 minutes longer than Reference Compound C.

In some embodiments, the 15-PGDH inhibitors of the present disclosure (e.g., formula I-VIII) have a mouse microsome stability T_{1/2} of greater than 10 minutes. In some embodiments, the 15-PGDH inhibitors of the present disclosure have a mouse microsome stability T_{1/2} of greater than 50 minutes. In some embodiments, the 15-PGDH inhibitors of the present disclosure have mouse microsome stability T_{1/2} of greater than 60 minutes. In some embodiments, the 15-PGDH inhibitors of the present disclosure have a mouse microsome stability T_{1/2} of greater than 70 minutes. In some embodiments, the 15-PGDH inhibitors of the present disclosure have a mouse microsome stability T_{1/2} of greater than 80 minutes. In some embodiments, the 15-PGDH inhibitors of the present disclosure have a mouse microsome stability T_{1/2} of greater than 90 minutes. In some embodiments, the 15-PGDH inhibitors of the present disclosure have a mouse microsome stability T_{1/2} of greater than 100 minutes. In some embodiments, the 15-PGDH inhibitors of the present disclosure have a mouse microsome stability T_{1/2} of greater than 110 minutes. In some embodiments, the 15-PGDH inhibitors of the present disclosure have a mouse microsome stability T_{1/2} of greater than 120 minutes. In some embodiments, the 15-PGDH inhibitors of the present disclosure have a mouse microsome stability T_{1/2} of greater than 130 minutes. In some embodiments, the 15-PGDH inhibitors of the present disclosure have a mouse microsome stability T_{1/2} of greater than 140 minutes. In some embodiments, the 15-PGDH inhibitors of the present disclosure have a mouse microsome stability T_{1/2} of greater than 145 minutes. In some embodiments, the 15-PGDH inhibitors of the present disclosure have a mouse microsome stability T_{1/2} ranging from 65 to at least 145 (e.g., 65, 70, 80, 90, 100, 110, 120, 130, 140, 150, 160, 170, 180, 190, 200, or more, including all values and ranges therebetween).

In some embodiments, the 15-PGDH inhibitors of the present disclosure (e.g., formula I-VIII) have a mouse microsome stability T_{1/2} which is at least 10 minutes longer than Reference Compound C (Example B). In some embodiments, the 15-PGDH inhibitors of the present disclosure have a mouse microsome stability T_{1/2} which is at least 20 minutes longer than Reference Compound C. In some embodiments, the 15-PGDH inhibitors of the present disclosure have a mouse microsome stability T_{1/2} which is at least 30 minutes longer than Reference Compound C. In some embodiments, the 15-PGDH inhibitors of the present disclosure have a mouse microsome stability T_{1/2} which is at least 40 minutes longer than Reference Compound C. In some embodiments, the 15-PGDH inhibitors of the present disclosure have a mouse microsome stability T_{1/2} which is at least 50 minutes longer than Reference Compound C. In some embodiments, the 15-PGDH inhibitors of the present disclosure have a mouse microsome stability T_{1/2} which is at least 60 minutes longer than Reference Compound C. In some embodiments, the 15-PGDH inhibitors of the present disclosure have a mouse microsome stability T_{1/2} which is at least 70 minutes longer than Reference Compound C. In some embodiments, the 15-PGDH inhibitors of the present disclosure have a mouse microsome stability T_{1/2} which is at least 80 minutes longer than Reference Compound C. In some embodiments, the 15-PGDH inhibitors of the present disclosure have a mouse microsome stability T_{1/2} which is at least 90 minutes longer than Reference Compound C. In some embodiments, the 15-PGDH inhibitors of the present disclosure have a mouse microsome stability T_{1/2} which is at least 100 minutes longer than Reference Compound C. In some embodiments, the 15-PGDH inhibitors of the present disclosure have a mouse microsome stability T_{1/2} which is at least 110 minutes longer than Reference Compound C. In some embodiments, the 15-PGDH inhibitors of the present disclosure have a mouse microsome stability T_{1/2} which is at least 120 minutes longer than Reference Compound C. In some embodiments, the 15-PGDH inhibitors of the present disclosure have a mouse microsome stability T_{1/2} which is at least 130 minutes longer than Reference Compound C. In some embodiments, the 15-PGDH inhibitors of the present disclosure have a mouse microsome stability T_{1/2} which is at least 140 minutes longer than Reference Compound C. In some embodiments, the 15-PGDH inhibitors of the present disclosure have a mouse microsome stability T_{1/2} that is from 65 to at least 145 longer than Reference Compound C (e.g., 65, 70, 80, 90, 100, 110, 120, 130, 140, 150, 160, 170, 180, 190, 200, or more, including all values and ranges therebetween).

The EC₅₀ for induction of PGE₂ is determined using A549 cells that have been treated with IL1-β 24 hours. In some embodiments, the 15-PGDH inhibitors of the present disclosure (e.g., formula I-VIII) have an EC₅₀ for induction of PGE₂ that is less than or equal to 10 nM. In some embodiments, the EC₅₀ is less than or equal to 5 nM. In some embodiments, the EC₅₀ is less than or equal to 4 nM. In some embodiments, the EC₅₀ is less than or equal to 3 nM. In some embodiments, the EC₅₀ is less than or equal to 2 nM. In some embodiments, the EC₅₀ is less than or equal to 1 nM. In some embodiments, the EC₅₀ is from 10 nM to about 0.01 nM including all values and subranges in between these values). In some embodiments, the EC₅₀ is at least 4 times less than the previously disclosed 15-PGDH inhibitors, such as those disclosed in the publications referenced above. In some embodiments, the EC₅₀ is at least 8 times less than the previously disclosed 15-PGDH inhibitors. In some embodiments, the EC₅₀ is at least 10 times less than the previously disclosed 15-PGDH inhibitors. In some embodiments, the EC₅₀ is at least 15 times less than the previously disclosed 15-PGDH inhibitors. In some embodiments, the EC₅₀ is at least 20 times less than the previously disclosed 15-PGDH inhibitors. In some embodiments, the EC₅₀ is at least 30 times less than the previously disclosed 15-PGDH inhibitors. In some embodiments, the EC₅₀ is at least 40 times less than the previously disclosed 15-PGDH inhibitors. In some embodiments, the EC₅₀ is at least 50 times less than the previously disclosed 15-PGDH inhibitors. In some embodiments, the EC₅₀ is 10 times to 50 times less than the previously disclosed 15-PGDH inhibitors.

In some embodiments, the EC₅₀ of the 15-PGDH inhibitors of the present disclosure (e.g., formula I-VIII) is at least 5 times less than Reference Compound C (Example B). In some embodiments, the EC₅₀ is at least 10 times less than Reference Compound C. In some embodiments, the EC₅₀ is at least 15 times less than Reference Compound C. In some embodiments, the EC₅₀ is at least 20 times less than Reference Compound C. In some embodiments, the EC₅₀ is at least 25 times less than Reference Compound C. In some embodiments, the EC₅₀ is at least 30 times less than Reference Compound C. In some embodiments, the EC₅₀ is at least 35 times less than Reference Compound C. In some embodiments, the EC₅₀ is at least 40 times less than Reference Compound C. In some embodiments, the EC₅₀ is at least 45 times less than Reference Compound C. In some embodiments, the EC₅₀ is at least 50 times less than Reference Compound C. In some embodiments, the EC₅₀ is 5 times to 50 times less than Reference Compound C.

In certain embodiments, the 15-PGDH inhibitor having formula (I-VIII), can be selected that can ia) at 2.5 μM concentration, stimulate a Vaco503 reporter cell line expressing a 15-PGDH luciferase fusion construct to a luciferase output level of greater than 70 (using a scale on which a value of 100 indicates a doubling of reporter output over baseline); iia) at 2.5 μM concentration stimulate a V9m reporter cell line expressing a 15-PGDH luciferase fusion construct to a luciferase output level of greater than 75; iiia) at 7.5 μM concentration stimulate a LS174T reporter cell line expressing a 15-PGDH luciferase fusion construct to a luciferase output level of greater than 70; and iva) at 7.5 μM concentration, does not activate a negative control V9m cell line expressing TK-renilla luciferase reporter to a level greater than 20; and va) inhibits the enzymatic activity of recombinant 15-PGDH protein at an IC₅₀ of less than 1 μM.

In other embodiments, the 15-PGDH inhibitor can ib) at 2.5 μM concentration, stimulate a Vaco503 reporter cell line expressing a 15-PGDH luciferase fusion construct to increase luciferase output; iib) at 2.5 μM concentration stimulate a V9m reporter cell line expressing a 15-PGDH luciferase fusion construct to increase luciferase output; iiib) at 7.5 μM concentration stimulate a LS174T reporter cell line expressing a 15-PGDH luciferase fusion construct to increase luciferase output; ivb) at 7.5 μM concentration, does not activate a negative control V9m cell line expressing TK-renilla luciferase reporter to a luciferase level greater than 20% above background; and vb) inhibits the enzymatic activity of recombinant 15-PGDH protein at an IC₅₀ of less than 1 μM.

In some embodiments, the compound or 15-PGDH inhibitor can inhibit the enzymatic activity of recombinant 15-PGDH at an IC₅₀ of less than 1 μM, at an IC₅₀ of less than 250 nM, at an IC₅₀ of less than 50 nM, at an IC₅₀ of less than 10 nM, at an IC₅₀ of less than 5 nM at a recombinant, at an IC₅₀ of about 2.5 nM to about 10 nM, or less than about 2.5 nM at a 15-PGDH concentration of about 5 nM to about 10 nM.

The 15-PGDH inhibitors described herein can be used for the prevention or the treatment of diseases that are associated with 15-PGDH and/or decreased prostaglandin levels and/or where it desirable to increase prostaglandin levels in the subject. For example, as discussed above, it is known that prostaglandins play an important role in hair growth. Specifically, internal storage of various types (A₂, F₂ₐ, E₂) of prostaglandins in the various compartments of hair follicles or their adjacent skin environments has been shown to be essential in maintaining and increasing hair density (Colombe L et. al, 2007, Exp. Dermatol, 16(9), 762-9). It has been reported that 15-PGDH, which is involved in the degradation of prostaglandins is present in the hair follicle dermal papillae, inactivates prostaglandins, especially, PGF₂ₐ and PGE₂, to cause scalp damage and alopecia (Michelet J F et. al., 2008, Exp. Dermatol, 17(10), 821-8). Thus, the compounds described herein, which have a suppressive or inhibitory activity against 15-PGDH that degrades prostaglandins, can improve scalp damage, prevent alopecia and promote hair growth and be used in a pharmaceutical composition for the prevention of alopecia and the promotion of hair growth.

In other embodiments, the 15-PGDH inhibitors described herein can be used in a pharmaceutical composition for promoting and/or inducing and/or stimulating pigmentation of the skin and/or skin appendages, and/or as an agent for preventing and/or limiting depigmentation and/or whitening of the skin and/or skin appendages, in particular as an agent for preventing and/or limiting canities.

In some embodiments, the 15-PGDH inhibitor can be applied to skin of a subject, e.g., in a topical application, to promote and/or stimulate pigmentation of the skin and/or hair growth, inhibit hair loss, and/or treat skin damage or inflammation, such as skin damage caused by physical or chemical irritants and/or UV-exposure.

In still other embodiments, the 15-PGDH inhibitors described herein can be used in a pharmaceutical composition for the prevention or the treatment of cardiovascular disease and/or diseases of vascular insufficiency, such as Raynaud's disease, Buerger's disease, diabetic neuropathy, and pulmonary artery hypertension. Prostaglandins including prostaglandin homologues produced in the body have been known to maintain the proper action of the blood vessel wall, especially to contribute to vasodilation for blood flow, preventing platelet aggregation and modulating the proliferation of smooth muscle that surrounds blood vessel walls (Yan. Cheng et. al., 2006, J. Clin., Invest). In addition, the inhibition of prostaglandins production or the loss of their activity causes the degeneration of the endothelium in the blood vessel walls, platelet aggregation and the dysfunction of cellular mechanism in the smooth muscle. Among others, the production of prostaglandins in blood vessels was shown to be decreased in hypertension patients, including pulmonary artery hypertension.

In other embodiments, the 15-PGDH inhibitors described herein can be used in a pharmaceutical composition for the prevention or the treatment of oral, intestinal, and/or gastrointestinal injury or diseases, or inflammatory bowel disease, such as oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, i.e., the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Moreover, 15-PGDH inhibitors would also be expected to protect from other forms of intestinal injury, such as toxicity from radiation, toxicity from chemotherapy, and chemotherapy induced mucositis.

In the kidney, prostaglandins modulate renal blood flow and may serve to regulate urine formation by both renovascular and tubular effects. In clinical studies, PGE₁ has been used to improve creatinine clearance in patients with chronic renal disease, to prevent graft rejection and cyclosporine toxicity in renal transplant patients, to reduce the urinary albumin excretion rate and N-acetyl-beta-D-glucosaminidase levels in patients with diabetic nephropathy (see Porter, Am., 1989, J. Cardiol., 64: 22E-26E). In addition, U.S. Pat. No. 5,807,895 discloses a method of preventing renal dysfunction by intravenous administration of prostaglandins such as PGE₁, PGE₂ and PGI₂. Furthermore, it has been reported that prostaglandins serve as vasodilators in the kidney, and, thus, the inhibition of prostaglandin production in the kidney results in renal dysfunction (Hao. C M, 2008, Annu Rev Physiol, 70, 357.about.77).

Thus, the 15-PGDH inhibitors described herein, which have a suppressive or inhibitory activity against 15-PGDH that degrades prostaglandins, may be effective in the prevention or the treatment of renal diseases that are associated with renal dysfunction.

The term “renal dysfunction” as used herein includes such manifestations as follows: lower than normal creatinine clearance, lower than normal free water clearance, higher than normal blood urea, nitrogen, potassium and/or creatinine levels, altered activity of kidney enzymes such as gamma glutamyl synthetase, alanine phosphatidase, N-acetyl-β-D-glucosaminidase, or β-w-microglobulin; and increase over normal levels of macroalbuminuria.

Prostaglandins including PGE₁, PGE₂ and PGF₂ₐ have also been shown to stimulate bone resorption and bone formation to increase the volume and the strength of the bone (H. Kawaguchi et. al., Clinical Orthop. Rel. Res., 313, 1995; J. Keller et al., Eur. Jr. Exp. Musculoskeletal Res., 1, 1992, 8692). Considering that 15-PGDH inhibits the activities of prostaglandins as mentioned above, the inhibition of 15-PGDH activity may lead to the promotion of bone resorption and bone formation that are inhibited by 15-PGDH. Thus, 15-PGDH inhibitors described herein can be effective for the promotion of bone resorption and bone formation by inhibiting 15-PGDH activity. 15-PGDH inhibitors can also be used to increase bone density, treat osteoporosis, promote healing of fractures, or promote healing after bone surgery or joint replacement, or to promote healing of bone to bone implants, bone to artificial implants, dental implants, and bone grafts.

In yet other embodiments, the 15-PGDH inhibitors described herein can be effective for treating 15-PGDH expressing cancers. Inhibition of 15-PGDH can inhibit the growth, proliferation, and metastasis of 15-PGDH expressing cancers.

In still other embodiments, the 15-PGDH inhibitors described herein can be effective for wound healing. Among various prostaglandins, PGE₂ is known to serve as a mediator for wound healing. Therefore, when skin is injured by wounds or burns, the inhibition of 15-PGDH activity can produce the treatment effect of the wounds or the burns by PGE₂.

Additionally, as discussed above, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in organs that would include liver, colon, and bone marrow. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, peripheral nerve.

Syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present disclosure, include, ischemia resulting from surgery, chemotherapy, radiation therapy, or cell, tissue, or organ transplant or graft.

In various embodiments, the methods described herein are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In some embodiments, the ischemia is associated with at least one of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient isc hemic attack (TIA), traumatic brain injury, ulcerative colitis, vascular disease of the kidney, vascular inflammatory conditions, von Hippel-Lindau syndrome, and wounds to tissues or organs.

In some embodiments, the 15-PGDH inhibitor can be administered to a preparation of hematopoietic stem cells, such as peripheral blood hematopoietic stem cells or umbilical cord stem cells of the subject, to increase the fitness of the stem cell preparation as a donor graft or to decrease the number of units of umbilical cord blood required for transplantation.

Hematopoietic stem cells are multipotent stem cells that give rise to all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al, U.S. Pat. No. 5,635,387; McGlave, et al, U.S. Pat. No. 5,460,964; Simmons, P., et al, U.S. Pat. No. 5,677,136; Tsukamoto, et al, U.S. Pat. No. 5,750,397; Schwartz, et al, U.S. Pat. No. 5,759,793; DiGuisto, et al, U.S. Pat. No. 5,681,599; Tsukamoto, et al, U.S. Pat. No. 5,716,827). Hematopoietic stem cells (HSCs) give rise to committed hematopoietic progenitor cells (HPCs) that are capable of generating the entire repertoire of mature blood cells over the lifetime of an organism.

Hematopoietic stem cells and hematopoietic progenitor cells are described herein generally as hematopoietic stem cells or hematopoietic stem and progenitor cells (HSPCs) unless noted otherwise and can refer to cells or populations identified by the presence of the antigenic marker CD34 (CD34⁺). In some embodiments, the hematopoietic stem cells can be identified by the presence of the antigenic marker CD34 and the absence of lineage (lin) markers and are therefore characterized as CD34⁺/lin⁻ cells.

The hematopoietic stem cells used in the methods described herein may be obtained from any suitable source of hematopoietic stem and progenitor cells and can be provided as a high purified population of hematopoietic stem cells or as composition that includes about 0.01% to about 100% of hematopoietic stem cells. For example, hematopoietic stem cells may be provided in compositions, such as unfractionated bone marrow (where the hematopoiectic stem cells comprise less than about 1% of the bone marrow cell population), umbilical cord blood, placental blood, placenta, fetal blood, fetal liver, fetal spleen, Wharton's jelly, or mobilized peripheral blood.

Sources of hematopoietic stem cells can be isolated or obtained from an organ of the body containing cells of hematopoietic origin. The isolated cells can include cells that are removed from their original environment. For example, a cell is isolated if it is separated from some or all of the components that normally accompany it in its native state. For example, an “isolated population of cells,” an “isolated source of cells,” or “isolated hematopoietic stem cells” and the like, as used herein, refer to in vitro or ex vivo separation of one or more cells from their natural cellular environment, and from association with other components of the tissue or organ, i.e., it is not significantly associated with in vivo substances.

Hematopoiectic stem cells can be obtained or isolated from bone marrow of adults, which includes femurs, hip, ribs, sternum, and other bones. Bone marrow aspirates containing hematopoiectic stem cells can be obtained or isolated directly from the hip using a needle and syringe. Other sources of hematopoietic stem cells include umbilical cord blood, placental blood, mobilized peripheral blood, Wharton's jelly, placenta, fetal blood, fetal liver, or fetal spleen. In particular embodiments, harvesting a sufficient quantity of hematopoietic stem cells for use in therapeutic applications may require mobilizing the stem and progenitor cells in the donor.

“Hematopoietic stem cell mobilization” refers to the release of stem cells from the bone marrow into the peripheral blood circulation for the purpose of leukapheresis, prior to stem cell transplantation. By increasing the number of stem cells harvested from the donor, the number of stem cells available for therapeutic applications can be significantly improved. Hematopoietic growth factors, e.g., granulocyte colony stimulating factor (G-CSF) or chemotherapeutic agents often are used to stimulate the mobilization. Commercial stem cell mobilization drugs exist and can be used in combination with G-CSF to mobilize sufficient quantities of hematopoietic stem and progenitor cells for transplantation into a subject. For example, G-CSF and Mozobil (Genzyme Corporation) can be administered to a donor in order to harvest a sufficient number of hematopoietic cells for transplantation. Other methods of mobilizing hematopoietic stem cells would be apparent to one having skill in the art.

In some embodiments, hematopoietic stem cells are obtained from umbilical cord blood. Cord blood can be harvested according to techniques known in the art {see, e.g., U.S. Pat. Nos. 7,147,626 and 7,131,958, herein incorporated by reference for such methodologies).

In one embodiment, hematopoietic stem cells can be obtained from pluripotent stem cell sources, e.g., induced pluripotent stem cells (iPSCs) and embryonic stem cells (ESCs). As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell. As used herein, the term “reprogramming” refers to a method of increasing the potency of a cell to a less differentiated state. As used herein, the term “programming” refers to a method of decreasing the potency of a cell or differentiating the cell to a more differentiated state.

In some embodiments, the hematopoietic stem cells can be administered or contacted ex vivo with one or more 15-PGDH inhibitors described herein to provide a therapeutic composition. In one embodiment, the therapeutic compositions can include a population of hematopoietic stem cells treated ex vivo with a one or more 15-PGDH inhibitor. In certain embodiments, the therapeutic composition comprising the enhanced hematopoietic stem cells is whole bone marrow, umbilical cord blood, or mobilized peripheral blood.

In particular embodiments, the therapeutic composition includes a population of cells, wherein the population of cells is about 95% to about 100% hematopoietic stem cells. The present disclosure contemplates, in part, that using therapeutic compositions of highly purified hematopoietic stem cells, e.g., a composition comprising a population of cells wherein the cells comprise about 95% hematopoietic stem cells, may improve the efficiency of stem cell therapies. Currently practiced methods of transplantations typically use unfractionated mixtures of cells where hematopoietic stem cells comprise less than 1% of the total cell population.

In some embodiments, the therapeutic composition comprises a population of cells, wherein the population of cells comprises less than about 0.1%, 0.5%, 1%, 2%, 5%, 10%, 15%, 20%, 25%, or 30% hematopoietic stem cells. The population of cells in some embodiments comprises less than about 0.1%, 0.5%, 1%, 2%, 5%, 10%, 15%, 20%, 25%, or 30% hematopoietic stem cells. In other embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% hematopoietic stem cells.

Hematopoietic stem cells in the therapeutic compositions of the present disclosure can be autologous/autogeneic (“self) or non-autologous (“non-self,” e.g., allogeneic, syngeneic or xenogeneic) relative to a subject to which the therapeutic composition is to be administered. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison.

Hematopoietic stem cells for use in the methods of the present disclosure may be depleted of mature hematopoietic cells such as T cells, B cells, NK cells, dendritic cells, monocytes, granulocytes, erythroid cells, and their committed precursors from bone marrow aspirate, umbilical cord blood, or mobilized peripheral blood (mobilized leukapheresis product). Mature, lineage committed cells are depleted by immunodepletion, for example, by labeling solid substrates with antibodies that bind to a panel of so-called “lineage” antigens: CD2, CD3, CD11b, CD14, CD15, CD16, CD79, CD56, CD123, and CD235a. A subsequent step can be performed to further purify the population of cells, in which a substrate labeled with antibodies that bind to the CD34⁺ antigen are used to isolate primitive hematopoietic stem cells. Kits are commercially available for purifying stem and progenitor cells from various cell sources and in particular embodiments, these kits are suitable for use with the methods described herein.

In one embodiment, the amount of hematopoietic stem cells in the therapeutic composition is at least 0.1×10⁵ cells, at least 0.5×10⁵ cells, at least 1×10⁵ cells, at least 5×10⁵ cells, at least 10×10⁵ cells, at least 0.5×10⁶ cells, at least 0.75×10⁶ cells, at least 1×10⁶ cells, at least 1.25×10⁶ cells, at least 1.5×10⁶ cells, at least 1.75×10⁶ cells, at least 2×10⁶ cells, at least 2.5×10⁶ cells, at least 3×10⁶ cells, at least 4×10⁶ cells, at least 5×10⁶ cells, at least 10×10⁶ cells, at least 15×10⁶ cells, at least 20×10⁶ cells, at least 25×10⁶ cells, or at least 30×10⁶ cells.

In one embodiment, the amount of hematopoietic stem cells in the therapeutic composition is the amount of hematopoietic stem cells in a partial or single cord of blood, or is at least 0.1×10⁵ cells/kg of bodyweight, at least 0.5×10⁵ cells/kg of bodyweight, at least 1×10⁵ cells/kg of bodyweight, at least 5×10⁵ cells/kg of bodyweight, at least 10×10⁵ cells/kg of bodyweight, at least 0.5×10⁶ cells/kg of bodyweight, at least 0.75×10⁶ cells/kg of bodyweight, at least 1×10⁶ cells/kg of bodyweight, at least 1.25×10⁶ cells/kg of bodyweight, at least 1.5×10⁶ cells/kg of bodyweight, at least 1.75×10⁶ cells/kg of bodyweight, at least 2×10⁶ cells/kg of bodyweight, at least 2.5×10⁶ cells/kg of bodyweight, at least 3×10⁶ cells/kg of bodyweight, at least 4×10⁶ cells/kg of bodyweight, at least 5×10⁶ cells/kg of bodyweight, at least 10×10⁶ cells/kg of bodyweight, at least 15×10⁶ cells/kg of bodyweight, at least 20×10⁶ cells/kg of bodyweight, at least 25×10⁶ cells/kg of bodyweight, or at least 30×10⁶ cells/kg of bodyweight.

Preparations of hematopoietic stem cells administered one or more 15-PGDH inhibitors and/or therapeutic compositions that include hematopoietic stem cells and one or more 15-PGDH inhibitor can be used for improving hematopoietic stem cell transplants and in treating ischemia or ischemia-damaged tissue, and in reducing further damage to ischemic tissue and/or repairing damage to ischemic tissue through cell recruitment, improving vascularization in ischemic tissue, improving tissue regeneration at sites of ischemia, decreasing ischemic tissue necrosis or apoptosis, and/or increasing cell survival at sites of ischemia. In particular embodiments, the preparations of 15-PGDH inhibitor treated hematopoietic stem cells and/or therapeutic compositions of 15-PGDH inhibitors and hematopoietic stem cells are useful to subjects in need of hematopoietic reconstitution, such as subjects that have undergone or are scheduled to undergo myeloablative therapy.

Subjects, which can be treated with the preparations of 15-PGDH inhibitor treated hematopoietic stem cells and/or therapeutic compositions of 15-PGDH inhibitors and hematopoietic stem cells, can include subjects that have or that have been diagnosed with various types of leukemias, anemias, lymphomas, myelomas, immune deficiency disorders, and solid tumors. A subject also includes a human who is a candidate for stem cell transplant or bone marrow transplantation, such as during the course of treatment for a malignant disease or a component of gene therapy. Subjects may also include individuals or animals that donate stem cells or bone marrow for allogeneic transplantation. In certain embodiments, a subject may have undergone myeloablative irradiation therapy or chemotherapy, or may have experienced an acute radiation or chemical insult resulting in myeloablation. In certain embodiments, a subject may have undergone irradiation therapy or chemotherapy, such as during various cancer treatments. Typical subjects include animals that exhibit aberrant amounts (lower or higher amounts than a “normal” or “healthy” subject) of one or more physiological activities that can be modulated by an agent or a stem cell or marrow transplant.

Subjects, which can be treated with the preparations of 15-PGDH inhibitor treated hematopoietic stem cells and/or therapeutic compositions of 15-PGDH inhibitors and hematopoietic stem cells, can also include subjects undergoing chemotherapy or radiation therapy for cancer, as well as subjects suffering from (e.g., afflicted with) non malignant blood disorders, particularly immunodeficiencies (e.g. SCID, Fanconi's anemia, severe aplastic anemia, or congenital hemoglobinopathies, or metabolic storage diseases, such as Hurler's disease, Hunter's disease, mannosidosis, among others) or cancer, particularly hematological malignancies, such as acute leukemia, chronic leukemia (myeloid or lymphoid), lymphoma (Hodgkin's or non-Hodgkin's), multiple myeloma, myelodysplastic syndrome, or non-hematological cancers such as solid tumors (including breast cancer, ovarian cancer, brain cancer, prostate cancer, lung cancer, colon cancer, skin cancer, liver cancer, or pancreatic cancer).

Subjects may also include subjects suffering from aplastic anemia, an immune disorder (severe combined immune deficiency syndrome or lupus), myelodysplasia, thalassemaia, sickle-cell disease or Wiskott-Aldrich syndrome. In some embodiments, the subject suffers from a disorder that is the result of an undesired side effect or complication of another primary treatment, such as radiation therapy, chemotherapy, or treatment with a bone marrow suppressive drug, such as zidovadine, chloramphenical or gangciclovir. Such disorders include neutropenias, anemias, thrombocytopenia, and immune dysfunction. Other subjects may have disorders caused by an infection (e.g., viral infection, bacterial infection or fungal infection) which causes damage to stem or progenitor cells of the bone marrow.

In addition, subjects suffering from the following conditions can also benefit from treatment using the preparations of 15-PGDH inhibitor treated hematopoietic stem cells and/or therapeutic compositions of 15-PGDH inhibitors and hematopoietic stem cells: lymphocytopenia, lymphorrhea, lymphostasis, erythrocytopenia, erthrodegenerative disorders, erythroblastopenia, leukoerythroblastosis; erythroclasis, thalassemia, myelodysplasia, myelofibrosis, thrombocytopenia, disseminated intravascular coagulation (DIC), immune (autoimmune) thrombocytopenic purpura (ITP), HIV inducted ITP, myelodysplasia; thrombocytotic disease, thrombocytosis, congenital neutropenias (such as Kostmann's syndrome and Schwachman-Diamond syndrome), neoplastic associated neutropenias, childhood and adult cyclic neutropaenia; post-infective neutropaenia; myelodysplastic syndrome; neutropaenia associated with chemotherapy and radiotherapy; chronic granulomatous disease; mucopolysaccharidoses; Diamond Blackfan Anemia; Sickle cell disease; or Beta thalassemia major.

In other embodiments, the preparations of 15-PGDH inhibitor treated hematopoietic stem cells and/or therapeutic compositions or 15-PGDH inhibitors and hematopoietic stem cells can be used in cell-based therapy for treating ischemic tissue or treating or ameliorating one or more symptoms associated with tissue ischemia, including, but not limited to, impaired, or loss of, organ function (including without limitation impairments or loss of brain, kidney, or heart function), cramping, claudication, numbness, tingling, weakness, pain, reduced wound healing, inflammation, skin discoloration, and gangrene.

In one embodiment, the subject exhibits at least one symptom of an ischemic tissue or tissue damaged by ischemia. In particular embodiments, the subject is a human who is has or who is at risk of having an ischemic tissue or tissue damaged by ischemia, e.g., a subject that has diabetes, peripheral vascular disease, thromboangiitis obliterans, vasculitis, cardiovascular disease, coronary artery disease or heart failure, or cerebrovascular disease, cardiovascular disease, or cerebrovascular disease.

Illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with ischemia, or increase the risk of ischemia in a subject, or cause a subject to exhibit more or more symptoms of ischemia suitable for treatment or amelioration using the methods of the present disclosure, include, ischemia resulting from surgery, chemotherapy, radiation therapy, or cell, tissue, or organ transplant or graft.

In various embodiments, the methods described herein are suitable for treating cerebrovascular ischemia, myocardial ischemia, limb ischemia (CLI), myocardial ischemia (especially chronic myocardial ischemia), ischemic cardiomyopathy, cerebrovascular ischemia, renal ischemia, pulmonary ischemia, intestinal ischemia, and the like.

In various embodiments, the present disclosure contemplates that the therapeutic cell compositions disclosed herein can be used to treat an ischemic tissue in which it is desirable to increase the blood flow, oxygen supply, glucose supply, or supply of nutrients to the tissue.

In some embodiments, the 15-PGDH inhibitor can be administered to a preparation of tissue stem cells, such as neural stem stems, mesenchymal stem cells, or stem cells that can generate other tissues, and/or a preparation of pluripotent stem cells.

In one embodiment, tissue stems cells can be obtained from pluripotent stem cell sources, e.g., iPSCs and ESCs.

In some embodiments, the tissue stem cells and/or pluripotent stem cells can be administered or contacted ex vivo with one or more 15-PGDH inhibitors described herein to provide a therapeutic composition. In one embodiment, the therapeutic compositions of the can include a population of tissue stem cells treated ex vivo with a one or more 15-PGDH inhibitor.

In particular embodiments, the therapeutic composition includes a population of cells, wherein the population of cells is about 95% to about 100% tissue stem cells. The present disclosure contemplates, in part, that using therapeutic compositions of highly purified tissue stem cells, e.g., a composition comprising a population of cells wherein the cells comprise about 95% tissue stem cells, may improve the efficiency of stem cell therapies.

In some embodiments, the therapeutic composition comprises a population of cells, wherein the population of cells comprises less than about 0.1%, 0.5%, 1%, 2%, 5%, 10%, 15%, 20%, 25%, or 30% tissue stem cells. The population of cells in some embodiments comprises less than about 0.1%, 0.5%, 1%, 2%, 5%, 10%, 15%, 20%, 25%, or 30% tissue stem cells. In other embodiments, the population of cells is about 0.1% to about 1%, about 1% to about 3%, about 3% to about 5%, about 10%-15%, about 15%-20%, about 20%-25%, about 25%-30%, about 30%-35%, about 35%-40%, about 40%-45%, about 45%-50%, about 60%-70%, about 70%-80%, about 80%-90%, about 90%-95%, or about 95% to about 100% tissue stem cells.

Tissue stem cells in the therapeutic compositions described herein can be autologous/autogeneic or non-autologous relative to a subject to which the therapeutic composition is to be administered.

Preparations of tissue stem cells administered one or more 15-PGDH inhibitors and/or therapeutic compositions that include tissue stem cells and one or more 15-PGDH inhibitor can be used for improving tissue stem cell transplants and in treating damaged tissue, and in reducing further tissue damage tissue and/or potentiating repair to damaged tissue through stem cell recruitment and/or increasing cell survival at sites of tissue damage.

Genetic disorders, syndromic conditions, traumatic injuries, chronic conditions, medical interventions, or other conditions that cause or are associated with tissue damage and a need for tissue repair, and thus, suitable for treatment or amelioration using the methods described herein, include, but are not limited to, acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerotic cardiovascular disease, autoimmune disease, bone fracture, brain edema, brain hypoperfusion, Buerger's disease, burn, cancer, cardiovascular disease, cartilage damage, cerebral infarct, cerebral ischemia, cerebral stroke, cerebrovascular disease, chemotherapy-induced neuropathy, chronic infection, chronic mesenteric ischemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetes, diabetic neuropathy, diabetes induced ischemia, disseminated intravascular coagulation (DIC), embolic brain ischemia, graft-versus-host disease, frostbite, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosarcoma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular disease of the kidney, vascular inflammatory conditions, von Hippel-Lindau syndrome, and wounds to tissues or organs.

Other illustrative examples of genetic disorders, syndromic conditions, traumatic injuries, chronic conditions, medical interventions, or other conditions that cause or are associated with tissue damage and a need for tissue repair suitable for treatment or amelioration using the methods of the present invention, include, ischemia resulting from surgery, chemotherapy, radiation therapy, or cell, tissue, or organ transplant or graft.

In other embodiments, the 15-PGDH inhibitor can be administered to a bone marrow graft donor or a hematopoietic stem cell donor to increase the fitness of a donor bone marrow graft or a donor hematopoietic stem cell graft.

In other embodiments, the 15-PGDH inhibitor can also be administered to bone marrow of a subject to increase stem cells in the subject or to increase the fitness of the marrow as a donor graft.

In yet other embodiments, the 15-PGDH inhibitor can be administered to a subject to mitigate bone marrow graft rejection, to enhance bone marrow graft engraftment, to enhance engraftment of a hematopoietic stem cell graft, or an umbilical cord blood stem cell graft, to enhance engraftment of a hematopoietic stem cell graft, or an umbilical cord stem cell graft, and/or to decrease the number of units of umbilical cord blood required for transplantation into the subject. The administration can be, for example, following treatment of the subject or the marrow of the subject with radiation therapy, chemotherapy, or immunosuppressive therapy.

In other embodiments, the 15-PGDH inhibitor can be administered to a recipient of a bone marrow transplant, of a hematopoietic stem cell transplant, or of an umbilical cord blood stem cell transplant, in order to decrease the administration of other treatments or growth factors.

In some embodiments, the 15-PGDH inhibitor can be administered to a subject to enhance recovery of neutrophils following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, neutropenias from other bone marrow diseases, drug induced neutropenia, immune neutropenias, idiopathic neutropenia, and following infections with viruses that include, but are not limited to, HIV, CMV, and parvovirus.

In other embodiments, the 15-PGDH inhibitor can be administered to a subject to enhance recovery of platelets following bone marrow transplantation, following umbilical cord blood transplantation, following transplantation with hematopoietic stem cells, following conventional chemotherapy, following radiation treatment, and in individuals with neutropenias from diseases that include but are not limited to aplastic anemia, myelodysplasia, myelofibrosis, thrombocytopenias from other bone marrow diseases, drug induced thrombocytopenia, immune thrombocytopenia, idiopathic thrombocytopenic purpura, idiopathic thrombocytopenia, and following infections with viruses that include, but are not limited to, HIV, CMV, and parvovirus.

In still other embodiments, the 15-PGDH inhibitor can be administered to a subject to enhance recovery of hemoglobin following bone marrow transplantation, following umbilical cord blood transplantation, following transplantation with hematopoietic stem cells, following conventional chemotherapy, following radiation treatment, and in individuals with anemias from diseases that include but are not limited to aplastic anemia, myelodysplasia, myelofibrosis, anemia from other bone marrow diseases, drug induced anemia, immune mediated anemias, anemia of chronic disease, idiopathic anemia, and following infections with viruses that include, but are not limited to, HIV, CMV, and parvovirus.

In some embodiments, the 15-PGDH inhibitor can be administered to a subject to enhance numbers of bone marrow stem cell numbers following bone marrow transplantation, following umbilical cord blood transplantation, following transplantation with hematopoietic stem cells, following conventional chemotherapy, following radiation treatment, in individuals with other bone marrow diseases, in individuals with cytopenias following viral infections, and in individuals with cytopenias.

In other embodiments, the 15-PGDH inhibitor can be administered to a subject to enhance response to cytokines administered to individuals with cytopenias that include but are not limited to neutropenia, thrombocytopenia, lymphocytopenia, and anemia. Cytokines whose responses may be enhanced by 15-PGDH inhibitors described herein include, but are not limited to: G-CSF, GM-CSF, EPO, IL-3, IL-6, TPO, SCF, and TPO-RA (thrombopoietin receptor agonist).

In further embodiments, the 15-PGDH inhibitor can be administered to a subject or to a tissue graft of a subject to mitigate graft rejection, to enhance graft engraftment, to enhance graft engraftment following treatment of the subject or the marrow of the subject with radiation therapy, chemotherapy, or immunosuppressive therapy, to confer resistance to toxic or lethal effects of exposure to radiation, confer resistance to the toxic effect of Cytoxan, the toxic effect of fludarabine, the toxic effect of chemotherapy, or the toxic effect of immunosuppressive therapy, to decrease infection, and/or to decrease pulmonary toxicity from radiation.

In other embodiments, the 15-PGDH inhibitor can be administered to a recipient of a tissue stem cell transplant, including but not limited to a transplant with hematopoietic stem cells, neural stem stems, mesenchymal stem cells, or stem cells for other tissues, so as to accelerate tissue regeneration and repair following the transplant.

In some embodiments, the administration of a 15-PGDH inhibitor can be in combination with G-CSF for the purpose of increasing neutrophils.

In other embodiments, the administration of a 15-PGDH inhibitor can be in combination with a hematopoietic cytokine for the purpose of increasing neutrophils.

In still other embodiments, the administration of a 15-PGDH inhibitor can be in combination with G-CSF for the purpose of increasing numbers of and/or of mobilizing peripheral blood hematopoietic stem cells.

In other embodiments, the administration of a 15-PGDH inhibitor can be in combination with a hemopoietic cytokine for the purpose of increasing numbers of and/or of mobilizing peripheral blood hematopoietic stem cells.

In some embodiments, the administration of a 15-PGDH inhibitor can be in combination with a second agent, including Plerixafor, for the purpose of increasing numbers of and/or of mobilizing peripheral blood hematopoietic stem cells.

In other embodiments, the administration of a 15-PGDH inhibitor can be in combination with G-CSF for the purpose of increasing numbers of and/or of mobilizing peripheral blood hematopoietic stem cells for use in hematopoietic stem cell transplantation.

In still other embodiments, the administration of a 15-PGDH inhibitor can be in combination with a hemopoietic cytokine for the purpose of increasing numbers of and/or of mobilizing peripheral blood hematopoietic stem cells for use in hematopoietic stem cell transplantation.

In other embodiments, the administration of a 15-PGDH inhibitor can be in combination with a second agent, including Plerixafor, for the purpose of increasing numbers of and/or of mobilizing peripheral blood hematopoietic stem cells for use in hematopoietic stem cell transplantation.

In still other embodiments, the administration of a 15-PGDH inhibitor can be in combination with G-CSF for the purpose of increasing numbers of hematopoietic stem cells in blood or bone marrow.

In other embodiments, the administration of a 15-PGDH inhibitor can be in combination with a hemopoietic cytokine for the purpose of increasing numbers of hematopoietic stem cells in blood or bone marrow.

In other embodiments, the 15-PGDH inhibitors can be used to treat and/or prevent fibrosis and various fibrotic diseases, disorders or conditions, and decrease fibrotic symptoms, such as collagen deposition, inflammatory cytokine expression, and inflammatory cell infiltration.

In some embodiments, a method of treating or preventing a fibrotic disease, disorder or condition includes administering to a subject in need thereof a therapeutically effect amount of a 15-PGDH inhibitor such that at least one symptom or feature of a fibrotic disease, disorder or condition, or other related diseases, disorders or conditions, is reduced in intensity, severity, or frequency, or has delayed onset.

As used herein, the term “fibrotic” diseases, disorders, or conditions include diseases, disorders, or conditions characterized, in whole or in part, by the excess production of fibrous material, including excess production of fibrotic material within the extracellular matrix, or the replacement of normal tissue elements by abnormal, non-functional, and/or excessive accumulation of matrix-associated components. The fibriotic diseases, disorders, or conditions, can include acute and chronic, clinical or subclinical presentation, in which fibrogenic associated biology or pathology is evident.

Examples of fibrotic diseases, disorders and conditions include systemic sclerosis, multifocal fibrosclerosis, nephrogenic systemic fibrosis, scleroderma(including morphea, generalized morphea, or linear scleroderma), sclerodermatous graft-vs-host-disease, kidney fibrosis (including glomerular sclerosis, renal tubulointerstitial fibrosis, progressive renal disease or diabetic nephropathy), cardiac fibrosis (e.g., myocardial fibrosis), pulomanry fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis, acute fibrosis, organ specific fibrosis, and the like.

Illustrative organ specific fibrotic disorders include, but are not limited to, pulmonary fibrosis, pulmonary hypertension, cystic fibrosis, asthma, chronic obstructive pulmonary disease, liver fibrosis, kidney fibrosis, NASH, and the like. Many fibrotic diseases, disorders or conditions have disordered and/or exaggerated deposition of extracellular matrix in affected tissues. Fibrosis may be associated with inflammation, occur as a symptom of underlying disease, and/or caused by surgical procedure or wound healing process. Unchecked fibrosis can result in destruction of the architecture of the underlying organ or tissue, commonly referred to as scarring.

In some embodiments, the 15-PGDH inhibitors can be used to treat or prevent lung fibrosis. The lung fibrosis can be selected from the group consisting of pulmonary fibrosis, pulmonary hypertension, chronic obstructive pulmonary disease (COPD), asthma, idiopathic pulmonary fibrosis, sarcoidosis, cystic fibrosis, familial pulmonary fibrosis, silicosis, asbestosis, coal worker's pneumoconiosis, carbon pneumoconiosis, hypersensitivity pneumonitides, pulmonary fibrosis caused by inhalation of inorganic dust, pulmonary fibrosis caused by an infectious agent, pulmonary fibrosis caused by inhalation of noxious gases, aerosols, chemical dusts, fumes or vapors, drug-induced interstitial lung disease, or pulmonary hypertension, and combinations thereof.

Pulmonary fibrosis is characterized by progressive scarring of lung tissue accompanied by fibroblast proliferation, excessive accumulation of extracellular matrix proteins, and abnormal alveolar structure. The thickened and stiff tissue makes it difficult for lungs to work properly, leading to breathing problems such as shortness of breath, and can ultimately be fatal. Pulmonary fibrosis may be caused by acute lung injury, viral infection, exposure to toxins, radiation, chronic disease, medications, or may be idiopathic (i.e., an undiscovered underlying cause).

The classic findings in idiopathic pulmonary fibrosis show diffuse peripheral scarring of the lungs with small bubbles (known as bullae) adjacent to the outer lining of the surface of the lung, often at the bases of the lungs. Idiopathic pulmonary fibrosis often has a slow and relentless progression. Early on, patients often complain of a dry unexplained cough. Next, shortness of breath (dyspnea) sets in and worsens over time triggered by less and less activity. Eventually, the shortness of breath becomes disabling, limiting all activity and even occurring while sitting still. In rarer cases, the fibrosis can be rapidly progressive, with dyspnea and disability occurring in weeks to months of onset of the disease. This form of pulmonary fibrosis has been referred to as Hamman-Rich syndrome.

Pulmonary hypertension is marked by an increase in the blood pressure of the lung vasculature, including the pulmonary artery, pulmonary vein, and/or pulmonary capillaries. Abnormally high pressure strains the right ventricle of the heart, causing it to expand. Over time, the right ventricle can weaken and lose its ability to pump enough blood to the lungs, leading to the development of heart failure. Pulmonary hypertension can occur as a result of other medical conditions, such as chronic liver disease and liver cirrhosis; rheumatic disorders such as scleroderma or systemic lupus erythematosus (lupus); and lung conditions including tumors, emphysema, chronic obstructive pulmonary disease (COPD), and pulmonary fibrosis. Pulmonary fibrosis may lead to narrowing of pulmonary vasculature resulting in pulmonary hypertension.

Chronic Obstructive Pulmonary Disease (COPD) is a common lung disease that is often associated with chronic bronchitis or emphysema. Symptoms can often include cough, mucus build up, fatigue, wheezing, and respiratory infection.

Chronic bronchitis and emphysema are diseases of the lungs in which the airways become narrowed. This leads to a limitation of the flow of air to and from the lungs, causing shortness of breath (dyspnea). In clinical practice, COPD is defined by its characteristically low airflow on lung function tests.

Lung damage and inflammation in the large airways results in chronic bronchitis. In the airways of the lung, the hallmark of chronic bronchitis is an increased number (hyperplasia) and increased size (hypertrophy) of the goblet cells and mucous glands of the airway. As a result, there is more mucus than usual in the airways, contributing to narrowing of the airways and causing a cough with sputum. Microscopically there is infiltration of the airway walls with inflammatory cells. Inflammation is followed by scarring and remodeling that thickens the walls and also results in narrowing of the airways. As chronic bronchitis progresses, there is squamous metaplasia (an abnormal change in the tissue lining the inside of the airway) and fibrosis (further thickening and scarring of the airway wall). The consequence of these changes is a limitation of airflow and difficulty breathing.

Asthma is a chronic lung disease characterized by inflammation and constriction of the airways. Asthma causes recurring periods of wheezing, tightness of the chest, shortness of breath, and coughing. Swelling and overproduction of mucus can cause further airway constriction and worsening of symptoms. There is evidence that increased matrix degradation may occur in asthma, and this may contribute to mechanical changes in the airways in asthma (Roberts et al (1995) Chest 107:111 S-117S, incorporated herein by reference in its entirety. Treatment of extracellular matrix degradation may ameliorate symptoms of asthma.

Cystic fibrosis is a recessive multi-system genetic disease characterized by abnormal transport of chloride and sodium across epithelium, leading to thick, viscous secretions in the lungs, pancreas, liver, intestine and reproductive tract. Cystic fibrosis is caused by a mutation in the gene for the protein cystic fibrosis transmembrane conductance regulator (CFTR). Lung disease results from clogging of the airways due to mucus build-up, decreased mucociliary clearance, and resulting inflammation, which can cause fibrotic injury and structural changes to the lungs. The fibrotic lung damage progresses over time leading some cystic fibrosis patients to require lung transplant.

Common symptoms of subjects suffering from cystic fibrosis include, but are not limited to, accumulation of thick mucus, copious phlegm production, frequent chest infections, frequent coughing, frequent shortness of breath, inflammation, decreased ability to exercise, opportunistic infections of the lung and sinus (including but not limited to Staphylococcus aureus, Haemophilus influenzae, Mycobacterium aviium, and Pseudomonas aeruginosa), pneumonia, tuberculosis, bronchiectasis, hemoptysis, pulmonary hypertension (and resulting heart failure), hypoxia, respiratory failure, allergic bronchopulmonary aspergillosis, mucus in the paranasal sinuses, sinus infection, facial pain, fever, excessive nasal drainage, development of nasal polyps, cardiorespiratory complications, CF-related diabetes, rectal prolapse, pancreatitis, malabsorption, intestinal blockage, exocrine pancreatic insufficiency, bile duct blockage, and liver cirrhosis.

In other embodiments, the 15-PGDH inhibitors can be used to treat or prevent fibrotic diseases, disorders or conditions caused by post-surgical adhesion formation. Post-surgical adhesion formation is a common complication of surgery. The formation of adhesions, from mechanical damage, ischemia, and infections, can increase morbidity and mortality following surgery. Although refined surgical procedures can reduce the magnitude of adhesion formation, adhesions are rarely eviscerated and an effective adjunctive therapy is needed. Reducing the fibrosis associated with this process could reduce pain, obstruction and other complications of surgery and promote healing and recovery.

Wounds (e.g., lacerations, openings) in mammalian tissue result in tissue disruption and coagulation of the microvasculature at the wound face. Repair of such tissue represents an orderly, controlled cellular response to injury. Soft tissue wounds, regardless of size, heal in a similar manner. Tissue growth and repair are biologic systems wherein cellular proliferation and angiogenesis occur in the presence of an oxygen gradient. The sequential morphological and structural changes which occur during tissue repair have been characterized in detail and have in some instances been quantified (see e.g., Hunt, T. K., et al., “Coagulation and macrophage stimulation of angiogenesis and wound healing,” in The Surgical Wound, pp. 1-18, ed. F. Dineen & G. Hildrick-Smith (Lea & Febiger, Philadelphia: 1981)). The cellular morphology consists of three distinct zones. The central avascular wound space is oxygen deficient, acidotic and hypercarbic, and has high lactate levels. Adjacent to the wound space is a gradient zone of local anemia (ischemia) which is populated by dividing fibroblasts. Behind the leading zone is an area of active collagen synthesis characterized by mature fibroblasts and numerous newly-formed capillaries (i.e., neovascularization). U.S. Pat. Nos. 5,015,629 and 7,022,675 (each incorporated by reference herein) disclose methods and compositions for increasing the rate of wound repair.

In some embodiments, the 15-PGDH inhibitors can used for reducing or preventing scar formation in a subject by administering to a subject in need of treatment. Scar formation is a natural part of the healing process. Disorderly collagen synthesis and deposition in a wound can result in excessive, thick, or raised scar formation. Generally, the larger the wound, the longer it takes to heal and the greater the chance of a problematic scar.

In other embodiments, the 15-PGDH inhibitors can be used to reduce or prevent scar formation on skin or scleroderma. There are several types of scars on skin. Hypertropic scars are raised, pinkish-red areas located inside the borders of the original injury. They are often described as itchy. In some cases, hypertropic scars shrink and fade on their own. Keloids are raised, deep-red areas that tend to cover much more area than that of the original injury. Even when surgically removed, keloids tend to recur. Atrophic scars are skin depressions, like those that sometimes form from severe acne. They are caused by inflammation that destroys the collagen during the rebuilding process, leaving an area of indentation.

In some embodiments, the 15-PGDH inhibitors can be used to treat or prevent systemic sclerosis. Systemic sclerosis is a systemic connective tissue disease characterized by alterations of the microvasculature, disturbances of the immune system and by massive deposition of collagen and other matrix substances in the connective tissue. Systemic sclerosis is a clinically heterogeneous generalized disorder which affects the connective tissue of the skin and internal organs such as gastrointestinal tract, lungs, heart and kidneys. Reduction of fibrosis resulting from systemic sclerosis may ameliorate symptoms and/or prevent further complications in affected tissues.

In other embodiments, the 15-PGDH inhibitors can be used to treat or prevent liver fibrosis. Liver fibrosis can result from a chronic liver disease, viral induced hepatic cirrhosis, hepatitis B virus infection, hepatitis C virus infection, hepatitis D virus infection, schistosomiasis, primary biliary cirrhosis, alcoholic liver disease or non-alcoholic steatohepatitis (NASH), NASH associated cirrhosis obesity, diabetes, protein malnutrition, coronary artery disease, auto-immune hepatitis, cystic fibrosis, α-1-antitrypsin deficiency, primary biliary cirrhosis, drug reaction and exposure to toxins.

Nonalcoholic steatohepatitis (NASH) is a common liver disease. It resembles alcoholic liver disease but occurs in people who drink little or no alcohol. The major feature in NASH is fat in the liver, along with inflammation and damage. Nevertheless, NASH can be severe and can lead to cirrhosis, in which the liver is permanently damaged and scarred and no longer able to work properly.

NASH is usually a silent disease with few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases may even begin to reverse on its own without specific therapy. Or NASH can slowly worsen, causing scarring or fibrosis to appear and accumulate in the liver. As fibrosis worsens, cirrhosis develops in which the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

In some embodiments, the 15-PGDH inhibitors can be used to treat or prevent kidney fibrosis. Kidney fibrosis can result from dialysis following kidney failure, catheter placement, a nephropathy, glomerulosclerosis, glomerulonephritis, chronic renal insufficiency, acute kidney injury, end stage renal disease or renal failure.

Kidney (renal) fibrosis results from excessive formation of fibrous connective tissue in the kidney. Kidney fibrosis causes significant morbidity and mortality and leads to a need for dialysis or kidney transplantation. Fibrosis can occur in either the filtering or reabsorptive component of the nephron, the functional unit of the kidney. A number of factors may contribute to kidney scarring, particularly derangements of physiology involved in the autoregulation of glomerular filtration. This in turn leads to replacement of normal structures with accumulated extracellular matrix. A spectrum of changes in the physiology of individual cells leads to the production of numerous peptide and non-peptide fibrogens that stimulate alterations in the balance between extracellular matrix synthesis and degradation to favor scarring.

In some embodiments, the symptoms of fibrosis of a tissue organ can comprise inflammation. In these embodiments, a therapeutically effective amount of the 15-PGDH inhibitor administered to the subject in need thereof can be an amount effective to decrease or reduce inflammatory cell count in the tissue or organ. A relevant sample can be obtained from the subject to determine the decrease or reduction in inflammatory cell count. In a non-limiting embodiment, the beneficial effect may be assessed by demonstrating a reduction in neutrophil count in BAL fluid from the subject with cystic fibrosis. The excessive recruitment of neutrophils into the airways of patients with CF is a significant predictor of lung disease severity in CF and therefore is an important therapeutic target. Methods for measuring such cell counts are well known in the art, including but not limited to FACS techniques. In some embodiments, the method may comprise reducing neutrophil cell count in BAL fluid from the subject compared to control. Any suitable control can be used for comparison, such as cystic fibrosis subjects not treated the 15-PGDH inhibitors. In some embodiments, a decrease in inflammatory cell count, such as neutrophil count, provides a clinical benefit to the subject. In various embodiments, the reduction in inflammatory cell count is at least 5%, 10%, 15%, 20%, 25%, 50%, or more compared to control.

In another embodiment, the beneficial effect of the 15-PGDH inhibitors may be assessed by a reduction in one or more inflammatory biomarkers in a relevant sample from the subject. In various non-limiting embodiments, the inflammatory biomarker may comprise or consist of one or more of cytokines or inflammatory cytokines associated with fibrosis. Such cytokines can include, for example, IL1β, MIP2 (e.g., CCL3 or CCL4), IFNδ, TGFβ, TNFα, IL-6, MCP-1, IL2, and IL-10 in BAL fluid. Methods for measuring the amount of such biomarkers are well known in the art, including but not limited to ELISAs. Thus, in this embodiment, the methods may further comprise the reducing an amount of one or more inflammatory biomarkers in a sample from the subject compared to control.

In other embodiments, the 15-PGDH inhibitors can be used in a method for decreasing or reducing collagen secretion or collagen deposition in a tissue or organ, such as the lung, the liver, the skin or the heart, of a subject. The method can include administering a therapeutically effective amount of the 15-PGDH inhibitors to the subject in need thereof. The subject can have or be at risk of an excessive collagen secretion or collagen deposition in the tissue or organ, such as the kidney, the lung, the liver, the intestines, the colon, the skin or the heart. Usually, the excessive collagen secretion or collagen deposition in an organ results from an injury or an insult. Such injury and insult are organ-specific. The 15-PGDH inhibitors can be administered over a sufficient period of time to decrease or reduce the level of collagen deposition in the tissue or organ, completely or partially. A sufficient period of time can be during one week, or between 1 week to 1 month, or between 1 to 2 months, or 2 months or more. For chronic condition, the 15-PGDH inhibitors can be advantageously administered for life time period.

15-PGDH inhibitors used to treat the fibrotic disease, disorder or condition and/or reduce collagen deposition can be identified using assays in which putative inhibitor compounds are applied to cells expressing 15-PGDH and then the functional effects on 15-PGDH activity are determined. Samples or assays comprising 15-PGDH that are treated with a potential inhibitor are compared to control samples without the inhibitor to examine the extent of effect. Control samples (untreated with modulators) are assigned a relative 15-PGDH activity value of 100%. Inhibition of 15-PGDH is achieved when the 15-PGDH activity value relative to the control is about 80%, optionally 50% or 25%, 10%, 5% or 1%. Additionally, in a model organism, PGE₂ signaling stimulates liver regeneration and increase survival after exposure to hepatoxic agents, such as acetaminophen. Hence, 15-PGDH inhibitors described herein may be utilized to increase liver regeneration after liver resection, in other settings that include after liver surgery, after live liver donation, or after receiving a liver transplant or to increase liver regeneration and increase survival after exposures to hepatoxic agents, including but not limited to acetaminophen and similar compounds.

PGE₁ analogues have also been used in the treatment of erectile dysfunction. Accordingly, in some embodiments, 15-PGDH inhibitors described herein can used either alone or combination with a prostaglandin for the treatment of erectile dysfunction.

Other embodiments described herein relate to the use of 15-PGDH inhibitors in combination with corticosteroids to treat inflammation and/or reduce aberrant activity of the immune system in a subject in need thereof. It was found that corticosteroids administered to a subject can induce 15-PGDH expression in tissue of the subject. Administration of a 15-PGDH inhibitor in combination with a corticosteroid was found to enhance anti-inflammatory and/or immunosuppressive effects of the corticosteroid while attenuating corticosteroid induced adverse and/or cytotoxic effects. Treatment of inflammatory and/or immune disorders by administration of 15-PGDH inhibitors in combination with corticosteroids can increase therapeutic efficacy and can allow the corticosteroids to be administered, in some instances, at lower dosages to achieve similar effects, and, in other instances, at higher dosages and for prolonged periods of times with attenuated and/or reduced adverse or cytotoxic effects. Additional embodiments herein relate to the use of 15-PGDH inhibitors in combination with TNF alpha inhibitors to treat inflammation and/or reduce aberrant activity of the immune system in a subject in need thereof.

In some embodiments, the 15-PGDH inhibitors can be administered in combination with corticosteroids and/or TNF inhibitors to treat intestinal, gastrointestinal, or bowel disorders. The intestinal, gastrointestinal, or bowel disorders treated can include oral ulcers, gum disease, gastritis, colitis, ulcerative colitis, gastric ulcers, inflammatory bowel disease, and Crohn's disease. As described below, it was found that that inhibitors of short-chain dehydrogenase activity, such as 15-PGDH inhibitors, can be administered to a subject in need thereof alone or in combination with corticosteroids to treat intestinal, gastrointestinal, or bowel disorders, such as oral ulcers, gum disease, gastritis, colitis, ulcerative colitis, gastric ulcers, inflammatory bowel disease, and Crohn's disease.

The 15-PGDH inhibitors described herein can be used in a pharmaceutical composition for the prevention or the treatment of oral, intestinal, and/or gastrointestinal injury or diseases, or inflammatory bowel disease (IBD), such as Crohn's disease, oral ulcers, gum disease, gastritis, colitis, ulcerative colitis, and gastric ulcers. Gastritis and gastric ulcer, representatives of the gastrointestinal diseases, are defined as the conditions where gastrointestinal mucus membrane is digested by gastric acid to form ulcer. In the stomach walls generally consisting of mucosa, submucosa, muscle layer and serosa, gastric ulcer even damages submucosa and muscle layer, while gastritis damages mucosa only. Although the morbidity rates of gastritis and gastric ulcer are relatively high, the causes thereof have not been clarified yet. Until now, they are known to be caused by an imbalance between aggressive factors and defensive factors, that is, the increase in aggressive factors such as the increase in gastric acid or pepsin secretion, or the decrease in defensive factors such as structural or morphological deficit of the gastric mucus membrane, the decrease in mucus and bicarbonate ion secretion, the decrease in prostaglandin production, or the like.

Currently available therapeutic agents for gastritis and gastric ulcer comprise various drugs for strengthening the defensive factors such as an antacid, which does not affect, gastric acid secretion but neutralizes gastric acid that has been already produced, an inhibitor of gastric acid secretion, a promoter of prostaglandin secretion, and a coating agent for stomach walls. Especially, prostaglandins are known to be essential in maintaining the mechanism for protecting and defending gastric mucus membrane (Wallace J L., 2008, Physiol Rev., 88(4), 1547-65, S. J. Konturek et al., 2005, Journal of Physiology and Pharmacology, 56(5)). In view of the above, since the 15-PGDH inhibitors described herein show a suppressive or inhibitory activity against 15-PGDH, which degrades prostaglandins that protect gastric mucus membrane, they can be effective for the prevention or the treatment of gastrointestinal diseases, inter alia, gastritis and gastric ulcer.

Additionally, corticosteroids and TNF alpha antagonists are both used in the treatment of ulcerative colitis and IBD patients. In mouse models, 15-PGDH inhibitors speed healing of ulcerative colitis. We have found that administering corticosteroids to mice elevates levels of colon 15-PGDH, an effect that should reduce the therapeutic effectiveness of corticosteroids in colitis treatment. This suggests that combining a corticosteroid with a 15-PGDH inhibitor should be more effective in colitis (and IBD) treatment than using either agent alone.

Similarly, we have shown that TNF-alpha suppresses colon 15-PGDH expression. This suggests that TNF-alpha antagonists will increase colon 15-PGDH expression, an effect that should reduce the therapeutic effectiveness of corticosteroids in colitis treatment. This suggests that combining a TNF-alpha antagonist, e.g., the chimeric antibody REMICADE (infliximab), with a 15-PGDH inhibitor should be more effective in colitis (and IBD) treatment than using either agent alone.

In other embodiments, the 15-PGDH inhibitors and corticosteroids or 15-PGDH inhibitors and TNF inhibitors can be provided in a topical composition or formulation that is used to treat inflammation and/or aberrant immune system activity associated with medical conditions, such as atopic dermatitis, psoriasis, eczematous dermatitis, nummular dermatitis, irritant contact dermatitis, allergic contact dermatitis (such as poison ivy exposure, poison oak exposure, and poison sumac exposure), seborrheic dermatitis, stasis dermatitis, and other steroid responsive dermatoses.

In other embodiments, the 15-PGDH inhibitors and corticosteroids or 15-PGDH inhibitors and TNF inhibitors provided in a topical composition can be used to treat, for example, acne vulgaris, alopecia, alopecia greata, vitiligo, eczema, xerotic eczema, keratosis pilaris, lichen planus, lichen sclerosus, lichen striatus, lichen simplex chronicus, prurigo nodularis, discoid lupus erythematosus, lymphocytic infiltrate of Jessner/Kanof, lymphacytoma cutis, pyoderma gangrenosum, pruritis ani, sarcoidosis, chondrodermatitis nodularis helices, and other inflammatory dermatological disorders.

Medical conditions treated by the 15-PGDH inhibitors and corticosteroids or 15-PGDH inhibitors and TNF inhibitors can also include, for example, keloids, hypertrophic scars, pretibial myxedema and other infiltrative dermatological disorders. Additional medical conditions include, for example, granuloma annulare, necrobiosis lipoidica diabeticorum, sarcoidosis, and other noninfectious granulomas.

In still other embodiments, the 15-PGDH inhibitors described herein can be administered in combination with corticosteroids or TNF inhibitors for wound healing, tissue regeneration, and/or tissue repair. Among various prostaglandins, PGE₂ is known to serve as a mediator for wound healing. Therefore, subjects who are receiving steroids, including those healing of wounds from undergoing surgery, can be administered a 15-PGDH inhibitor to enhance PGE₂ and promote would healing.

Additionally, increased prostaglandin levels have been shown to stimulate signaling through the Wnt signaling pathway via increased beta-catenin mediated transcriptional activity. Wnt signaling is known to be a key pathway employed by tissue stem cells. Hence, 15-PGDH inhibitors described herein may be utilized to increase tissue stem cell numbers for purposes that would include promoting tissue regeneration or repair in subjects receiving corticosteroid treatment. In addition, 15-PGDH inhibitors described herein may be utilized to promote tissue regeneration or repair in additional organs that would include but are not limited to brain, eye, cornea, retina, lung, heart, stomach, small intestine, pancreas, beta-cells of the pancreas, kidney, bone, cartilage, and peripheral nerve.

In other embodiments, the 15-PGDH inhibitor can be used as a glucocorticoid sensitizer to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. Therapeutic effects of the 15-PGDH inhibitors when used as a glucocorticoid sensitizer include any, but are not limited to, steroid-sparing in corticosteroid-dependent patients, better responsiveness or tolerance to corticosteroids, achieving efficacy by using a lower dose of corticosteroid, preventing individuals at risk for developing refractory responses or resistance or exacerbations in response to antigen exposures, infections, exercise, or irritants, achieving optimal immune functions, easier responses for the subject or patient when steroid administration is tapered or withdrawn, or after prolonged administration of corticosteroids, decreased risks for developing corticosteroid-related adverse events such as opportunistic infections, bone loss, pathologic fracture, diabetes, cataract, and combinations thereof.

In some embodiments, the 15-PGDH inhibitor can be administered to a subject in combination with the corticosteroid to treat glucocorticoid insensitivity, restore corticosteroid sensitivity, enhance glucocorticoid sensitivity, and/or reverse the glucocorticoid insensitivity in a subject experiencing corticosteroid dependence or corticoid resistance or unresponsiveness or intolerance to corticosteroids. The glucocorticoid insensitivity related conditions can include a range of immune-inflammatory disorders/diseases treated with steroids when the therapy fails to achieve disease control or is not effective or intolerant or dependent to corticosteroids, and combinations thereof.

In other embodiments, the 15-PGDH inhibitor and corticosteroid or the 15-PGDH inhibitor and TNF inhibitor can be administered to a subject that exhibits one or more glucocorticoid insensitivity related diseases, disorders, or conditions selected from the group consisting of glucocorticoid resistant asthma, refractory rheumatoid arthritis, refractory inflammatory bowel disease, chronic obstructive pulmonary disease, acute respiratory distress syndrome, interstitial pulmonary fibrosis, cystic fibrosis, refractory ulcerative colitis, children with severe Crohn's disease, corticosteroid refractory asthma, desquamative interstitial pneumonia refractory to corticosteroid, refractory inflammatory myopathies, refractory myasthenia gravis, refractory pemphigus vulgaris, methotrexate-refractory RA patients, refractory nephrotic syndrome, refractory multiple sclerosis, refractory sprue-like disease, steroid-resistant sarcoidosis, refractory mucosal lesions of pemphigus vulgaris, refractory Schnitzler syndrome, resistant dermatitis of the head and neck, severe refractory atopic dermatitis, refractory Idiopathic thrombocytopenia purpura, refractory orbital myositis, refractory or recurrent lymphomas, critically ill patients with sepsis or acute respiratory distress syndrome (ARDS) and relative adrenal insufficiency, rosacea, polymyalgia rheumatic, giant cell arteritis, polymyositis, dermatomyositis, Kawasaki syndrome, Guillain-Barre syndrome, chronic inflammatory demyelinating polyneuropathy, multifocal motor neuropathy, Stiff man syndrome, corticosteroid dependent systemic lupus erythematosus, corticosteroid dependent multiple sclerosis, symptomatic corticosteroid dependent asthma, primary Sjogren's syndrome, systemic vasculitis, polymyositis, organ transplants, graft-versus-host disease, inflammatory diseases, autoimmune diseases, hyperproliferative diseases, lupus, osteoarthritis, rhinosinusitis, polyarteritis nodosa, Wegener's granulomatosis, giant cell arteritis, allergic rhinitis, urticaria, hereditary angioedema, tendonitis, bursitis, autoimmune chronic active hepatitis, cirrhosis, transplant rejection, psoriasis, dermatitus, malignancies, leukemia, myelomas, lymphomas, acute adrenal insufficiency, rheumatic fever, granulomatous disease, immune proliferation/apotosis, hypothalamic-pituitary-adrenal (HPA) axis suppression and regulation, hypercortisolemia, modulation of the Th1/Th2 cytokine balance, chronic kidney disease, spinal cord injury, cerebral edema, thrombocytopenia, Little's syndrome, Addison's disease, autoimmune hemolytic anemia, uveitis, pemphigus vulgaris, nasal polyps, sepsis, bacterial infections, viral infections, rickettsial infections, parasitic infections, type II diabetes, obesity, metabolic syndrome, depression, schizophrenia, mood disorders, Cushing's syndrome, anxiety, sleep disorders, memory and learning enhancement, glucocorticoid-induced glaucoma, atopic dermatitis, drug hypersensitivity reactions, serum sickness, bullous dermatitis herpetiformis, contact dermatitis, exfoliative erythroderma, mycosis fungoides, pemphigus, nonsuppurative thyroiditis, sympathetic ophthalmia, uveitis, ocular inflammatory conditions unresponsive to topical steroids, allergic bronchopulmonary aspergillosis, fulminating or disseminated pulmonary tuberculosis when used concurrently with appropriate chemotherapy, hypersensitivity pneumonitis, idiopathic bronchiolitis obliterans with organizing pneumonia, idiopathic eosinophilic pneumonias, idiopathic pulmonary fibrosis, Pneumocystis carinii pneumonia (PCP) associated with hypoxemia occurring in an HIV(+) individual who is also under treatment with appropriate anti-PCP antibiotics, a diuresis or remission of proteinuria in nephrotic syndrome, without uremia, of the idiopathic type or that due to lupus erythematosus, ankylosing spondylitis, polymyalgia rheumatic, psoriatic arthritis, relapsing polychondritis, trichinosis with neurologic or myocardial involvement, and tuberculous meningitis.

It will be appreciated that other 15-PGDH inhibitors can be used in the methods described described herein. These other 15-PGDH inhibitors can include known 15-PGDH inhibitors including, for example, tetrazole compounds of formulas (I) and (II), 2-alkylideneaminooxyacetamidecompounds of formula (I), heterocyclic compounds of fourmulas (VI) and (VII), and pyrazole compounds of formula (III) described in U.S. Patent Application Publication No. 2006/0034786 and U.S. Pat. No. 7,705,041; benzylidene-1,3-thiazolidine compounds of formula (I) described in U.S. Patent Application Publication No. 2007/0071699; phenylfurylmethylthiazolidine-2,4-dione and phenylthienylmethylthiazolidine-2,4-dione compounds described in U.S. Patent Application Publication No. 2007/0078175; thiazolidenedione derivatives described in U.S. Patent Application Publication No. 2011/0269954; phenylfuran, phenylthiophene, or phenylpyrrazole compounds described in U.S. Pat. No. 7,294,641; 5-(3,5-disubstituted phenylazo)-2-hydroxybenzene-acetic acids and salts; and lactones described in U.S. Pat. No. 4,725,676; azo compounds described in U.S. Pat. No. 4,889,846; and 15-PGHD inhibitors described in PCT/US2014/060761 and US Patent Application Publication No. 2015/0072998A1, all of which are herein incorporated by reference in their entirety.

The 15-PGDH inhibitors described herein can be provided in a pharmaceutical composition or cosmetic composition depending on the pathological or cosmetic condition or disorder being treated. A pharmaceutical composition containing the 15-PGDH inhibitors described herein as an active ingredient may be manufactured by mixing the derivative with a pharmaceutically acceptable carrier(s) or an excipient(s) or diluting the 15-PGDH inhibitors with a diluent in accordance with conventional methods. The pharmaceutical composition may further contain fillers, anti-cohesives, lubricants, wetting agents, flavoring agents, emulsifying agents, preservatives and the like. The pharmaceutical composition may be formulated into a suitable formulation in accordance with the methods known to those skilled in the art so that it can provide an immediate, controlled or sustained release of the 15-PGDH inhibitors after being administered into a mammal.

In some embodiments, the pharmaceutical composition may be formulated into a parenteral or oral dosage form. The solid dosage form for oral administration may be manufactured by adding excipient, if necessary, together with binder, disintegrants, lubricants, coloring agents, and/or flavoring agents, to the 15-PGDH inhibitors and shaping the resulting mixture into the form of tablets, sugar-coated pills, granules, powder or capsules. The additives that can be added in the composition may be ordinary ones in the art. For example, examples of the excipient include lactose, sucrose, sodium chloride, glucose, starch, calcium carbonate, kaolin, microcrystalline cellulose, silicate and the like. Exemplary binders include water, ethanol, propanol, sweet syrup, sucrose solution, starch solution, gelatin solution, carboxymethylcellulose, hydroxypropyl cellulose, hydroxypropyl starch, methylcellulose, ethylcellulose, shellac, calcium phosphonate and polypyrrolidone. Examples of the disintegrant include dry starch, sodium arginate, agar powder, sodium bicarbonate, calcium carbonate, sodium lauryl sulfate, stearic monoglyceride and lactose. Further, purified talc, stearates, sodium borate, and polyethylene glycol may be used as a lubricant; and sucrose, bitter orange peel, citric acid, tartaric acid, may be used as a flavoring agent. In some embodiments, the pharmaceutical composition can be made into aerosol formulations (e.g., they can be nebulized) to be administered via inhalation.

The 15-PGDH inhibitors described herein may be combined with flavoring agents, buffers, stabilizing agents, and the like and incorporated into oral liquid dosage forms such as solutions, syrups or elixirs in accordance with conventional methods. One example of the buffers may be sodium citrate. Examples of the stabilizing agents include tragacanth, acacia and gelatin.

In some embodiments, the 15-PGDH inhibitors described herein may be incorporated into an injection dosage form, for example, for a subcutaneous, intramuscular or intravenous route by adding thereto pH adjusters, buffers, stabilizing agents, relaxants, topical anesthetics. Examples of the pH adjusters and the buffers include sodium citrate, sodium acetate and sodium phosphate. Examples of the stabilizing agents include sodium pyrosulfite, EDTA, thioglycolic acid and thiolactic acid. The topical anesthetics may be procaine HCl, lidocaine HCl and the like. The relaxants may be sodium chloride, glucose and the like.

In other embodiments, the 15-PGDH inhibitors described herein may be incorporated into suppositories in accordance with conventional methods by adding thereto pharmaceutically acceptable carriers that are known in the art, for example, polyethylene glycol, lanolin, cacao butter or fatty acid triglycerides, if necessary, together with surfactants such as Tween.

The pharmaceutical composition may be formulated into various dosage forms as discussed above and then administered through various routes including an oral, inhalational, transdermal, subcutaneous, intravenous or intramuscular route. In some embodiments, the 15-PGDH inhibitors described herein can be administered orally, intravenously, or intraperitoneally. The dosage can be a pharmaceutically effective amount. The pharmaceutically effective amount can be, for example, an amount of the 15-PGDH inhibitor to treat or improve alopecia, cardiovascular disease, gastrointestinal disease, wounds, and renal disease. The pharmaceutically effective amount of the compound will be appropriately determined depending on the kind and the severity of the disease to be treated, age, sex, body weight and the physical condition of the patients to be treated, administration route, duration of therapy and the like. Generally, the effective amount of the compound may be in the range of about 1 to 1,000 mg in the oral administration, about 0.1 to 500 mg in the intravenous administration, about 5 to 1,000 mg in the rectal administration. Generally, the daily dosage for adults is in the range of about 0.1 to 5,000 mg, preferably about to 1,000 mg but cannot be determined uniformly because it depends on age, sex, body weight and the physical condition of the patients to be treated. The formulation may be administered once a day or several times a day with a divided dose.

Cosmetic compositions containing the 15-PGDH inhibitor can include any substance or preparation intended to be brought into contact with the various superficial parts of the human body (epidermis, body hair and hair system, nails, lips and external genital organs) or with the teeth or the buccal mucous membranes for the purpose, exclusively or mainly, of cleansing them, of giving them a fragrance, of modifying their appearance and/or of correcting body odors and/or protecting them or of maintaining them in good condition.

The cosmetic composition can comprise a cosmetically acceptable medium that may be water or a mixture of water and at least one solvent selected from among hydrophilic organic solvents, lipophilic organic solvents, amphiphilic organic solvents, and mixtures thereof.

For topical application, the cosmetic composition can be administered in the form of aqueous, alcoholic, aqueous-alcoholic or oily solutions or suspensions, or of a dispersion of the lotion or serum type, of emulsions that have a liquid or semi-liquid consistency or are pasty, obtained by dispersion of a fatty phase in an aqueous phase (O/W) or vice versa (W/O) or multiple emulsions, of a free or compacted powder to be used as it is or to be incorporated into a physiologically acceptable medium, or else of microcapsules or microparticles, or of vesicular dispersions of ionic and/or nonionic type. It may thus be in the form of a salve, a tincture, milks, a cream, an ointment, a powder, a patch, an impregnated pad, a solution, an emulsion or a vesicular dispersion, a lotion, aqueous or anhydrous gels, a spray, a suspension, a shampoo, an aerosol or a foam. It may be anhydrous or aqueous. It may also comprise solid preparations constituting soaps or cleansing cakes.

The cosmetic compositions may in particular comprise a hair care composition, and in particular a shampoo, a setting lotion, a treating lotion, a styling cream or gel, restructuring lotions for the hair, a mask, etc. The cosmetic compositions can be a cream, a hair lotion, a shampoo or a conditioner. These can be used in particular in treatments using an application that may or may not be followed by rinsing, or else in the form of a shampoo. A composition in the form of a foam, or else in the form of spray or an aerosol, then comprising propellant under pressure, is also intended. It can thus be in the form of a lotion, serum, milk, cream, gel, salve, ointment, powder, balm, patch, impregnated pad, cake or foam.

In particular, the compositions for application to the scalp or the hair can be in the form of a hair care lotion, for example for daily or twice-weekly application, of a shampoo or of a hair conditioner, in particular for twice-weekly or weekly application, of a liquid or solid soap for cleansing the scalp, for daily application, of a hairstyle shaping product (lacquer, hair setting product or styling gel), of a treatment mask, or of a foaming gel or cream for cleansing the hair. These may also be in the form of a hair dye or mascara to be applied with a brush or a comb.

Moreover, for topical application to the eyelashes or body hair, the compositions may be in the form of a pigmented or unpigmented mascara, to be applied with a brush to the eyelashes or alternatively to beard or moustache hair. For a composition administration by injection, the composition may be in the form of an aqueous lotion or an oily suspension. For oral use, the composition may be in the form of capsules, granules, oral syrups or tablets. According to a particular embodiment, the composition is in the form of a hair cream or hair lotion, a shampoo, a hair conditioner or a mascara for the hair or for the eyelashes.

In a known manner, the cosmetic compositions may also contain adjuvants that are normal in the cosmetics field, such as hydrophilic or lipophilic gelling agents, hydrophilic or lipophilic additives, preservatives, antioxidants, solvents, fragrances, fillers, UV-screening agents, odor absorbers and dyestuffs. The amounts of these various adjuvants are those conventionally used in the cosmetics field, and are for example from 0.1% to 20%, in particular less than or equal to 10%, of the total weight of the composition. According to their nature, these adjuvants can be introduced into the fatty phase, into the aqueous phase and/or into the lipid spherules.

In some embodiments, the 15-PGDH inhibitor can be administered in a combinatorial therapy or combination therapy that includes administration of a 15-PGDH inhibitor with one or more additional active agents. The phrase “combinatorial therapy” or “combination therapy” embraces the administration of the 15-PGDH inhibitor, and one or more therapeutic agents as part of a specific treatment regimen intended to provide beneficial effect from the co-action of these therapeutic agents. Administration of these therapeutic agents in combination typically is carried out over a defined period (usually minutes, hours, days or weeks depending upon the combination selected). “Combinatorial therapy” or “combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example by administering to the subject an individual dose having a fixed ratio of each therapeutic agent or in multiple, individual doses for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissue. The therapeutic agents can be administered by the same route or by different routes. The sequence in which the therapeutic agents are administered is not narrowly critical.

In some embodiments, the additional active agent can be chosen in particular from lipoxygenase inhibitors as described in EP 648488, the bradykinin inhibitors described in particular in EP 845700, prostaglandins and their derivatives, in particular those described in WO 98/33497, WO 95/11003, JP 97-100091, JP 96-134242, the agonists or antagonists of the receptors for prostaglandins, and the nonprostanoic analogues of prostaglandins as described in EP 1175891 and EP 1175890, WO 01/74307, WO 01/74313, WO 01/74314, WO 01/74315 or WO 01/72268.

In other embodiments, the 15-PGDH inhibitors can be administered in combination with active agents, such as vasodilators, prostanoid agonists, antiandrogens, cyclosporins and their analogues, antimicrobials, triterpenes, alone or as a mixture. The vasodilators can include potassium channel agonists including minoxidil and its derivatives, aminexil and the compounds described in U.S. Pat. Nos. 3,382,247, 5,756,092, 5,772,990, 5,760,043, 5,466,694, 5,438,058, 4,973,474, chromakalin and diazoxide. The antiandrogens can include 5.alpha.-reductase inhibitors such as finasteride and the compounds described in U.S. Pat. No. 5,516,779, cyprosterone acetate, azelaic acid, its salts and its derivatives, and the compounds described in U.S. Pat. No. 5,480,913, flutamide and the compounds described in U.S. Pat. Nos. 5,411,981, 5,565,467 and 4,910,226. The antimicrobial compounds can include selenium derivatives, ketoconazole, triclocarban, triclosan, zinc pyrithione, itraconazole, pyridine acid, hinokitiol, mipirocine, and the compounds described in EP 680745, clinycine hydrochloride, benzoyl or benzyl peroxide and minocycline. The anti-inflammatory agents can include inhibitors specific for Cox-2 such as for example NS-398 and DuP-697 (B. Batistini et al., DN&P 1994; 7(8):501-511) and/or inhibitors of lipoxygenases, in particular 5-lipoxygenase, such as for example zileuton (F. J. Alvarez & R. T. Slade, Pharmaceutical Res. 1992; 9(11):1465-1473).

Other active compounds, which can be present in pharmaceutical and/or cosmetic compositions can include aminexil and its derivatives, 60-[(9Z,12Z)octadec-9,12-dienoyl]hexapyranose, benzalkonium chloride, benzethonium chloride, phenol, oestradiol, chlorpheniramine maleate, chlorophyllin derivatives, cholesterol, cysteine, methionine, benzyl nicotinate, menthol, peppermint oil, calcium panthotenate, panthenol, resorcinol, protein kinase C inhibitors, prostaglandin H synthase 1 or COX-1 activators, or COX-2 activators, glycosidase inhibitors, glycosaminoglycanase inhibitors, pyroglutamic acid esters, hexosaccharidic or acylhexosaccharidic acids, substituted ethylenearyls, N-acylated amino acids, flavonoids, derivatives and analogues of ascomycin, histamine antagonists, triterpenes, such as ursolic acid and the compounds described in U.S. Pat. Nos. 5,529,769, 5,468,888, 5,631,282, saponins, proteoglycanase inhibitors, agonists and antagonists of oestrogens, pseudopterins, cytokines and growth factor promoters, IL-1 or IL-6 inhibitors, IL-10 promoters, TNF inhibitors, vitamins, such as vitamin D, analogues of vitamin B12 and panthotenol, hydroxy acids, benzophenones, esterified fatty acids, and hydantoin.

Pharmaceutical and/or cosmetic compositions including the 15-PGDH inhibitor described herein can additionally contain, for example, at least one compound chosen from prostaglandins, in particular prostaglandin PGE₁, PGE₂, their salts, their esters, their analogues and their derivatives, in particular those described in WO 98/33497, WO 95/11003, JP 97-100091, JP 96-134242, in particular agonists of the prostaglandin receptors. It may in particular contain at least one compound such as the agonists (in acid form or in the form of a precursor, in particular in ester form) of the prostaglandin F₂α receptor, such as for example latanoprost, fluprostenol, cloprostenol, bimatoprost, unoprostone, the agonists (and their precursors, in particular the esters such as travoprost) of the prostaglandin E₂ receptors such as 17-phenyl PGE₂, viprostol, butaprost, misoprostol, sulprostone, 16,16-dimethyl PGE₂, 11-deoxy PGE₁, 1-deoxy PGE₁, the agonists and their precursors, in particular esters, of the prostacycline (IP) receptor such as cicaprost, iloprost, isocarbacycline, beraprost, eprostenol, treprostinil, the agonists and their precursors, in particular the esters, of the prostaglandin D₂ receptor such as BW245C ((4S)-(3-[(3R,S)-3-cyclohexyl-3-isopropyl]-2,5-dioxo)-4-imidazolidinehept-anoic acid), BW246C ((4R)-(3-[(3R,S)-3-cyclohexyl-3-isopropyl]-2,5-dioxo)-4-imidazolidinehept-anoic acid), the agonists and their precursors, in particular the esters, of the receptor for the thromboxanes A2 (TP) such as I-BOP ([1S-[1a,2a(Z), 3b(1E,3S),4a]]-7-[3-[3-hydroxy-4-[4-(iodophenoxy)-1-butenyl]-7-oxabicyclo-[2.2.1]hept-2-yl]-5-heptenoic acid).

Advantageously, the composition can include at least one 15-PGDH inhibitor as defined above and at least one prostaglandin or one prostaglandin derivative such as for example the prostaglandins of series 2 including in particular PGF_{2α} and PGE₂ in saline form or in the form of precursors, in particular of the esters (example isopropyl esters), their derivatives such as 16,16-dimethyl PGE₂, 17-phenyl PGE₂ and 16,16-dimethyl PGF₂ₐ 17-phenyl PGF_{2α}, prostaglandins of series 1 such as 11-deoxyprostaglandin E1, 1-deoxyprostaglandin E1 in saline or ester form, is their analogues, in particular latanoprost, travoprost, fluprostenol, unoprostone, bimatoprost, cloprostenol, viprostol, butaprost, misoprostol, their salts or their esters.

The invention is further illustrated by the following examples, which is not intended to limit the scope of the claims.

### EXAMPLES

### Example A. Analysis of 15-PGDH Inhibitors of the Present Invention

This Example provides data on 15-PGDH inhibitors using an assay described in U.S. Pat. No. 9,790,233, which is herein incorporated by reference in its entirety. The data categorizes the IC₅₀ of each compound for inhibiting enzymatic activity of recombinant 15-PGDH in an in vitro assay: <2.5 nM (***), ≥2.5 nM and ≤10 nM (**), or >10 nM (*). The Recombinant 15-PGDH is human unless otherwise specified.

TABLE 1

| | | PDGH |
|---|---|---|
| Molecule | | Assay- |
| Name | Structure | IC₅₀ |
| 1 | 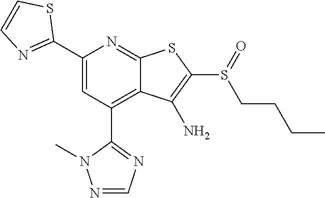 [SMILES: CCCCS(=O)c1sc2nc(-c3nccs3)cc(-c3ncnn3C)c2c1N] | *** |
| 2 | 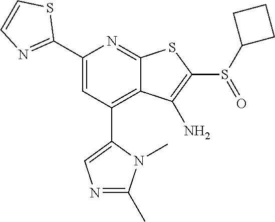 [SMILES: Cc1ncc(-c2cc(-c3nccs3)nc3sc(S(=O)C4CCC4)c(N)c23)n1C] | *** |
| 3 | 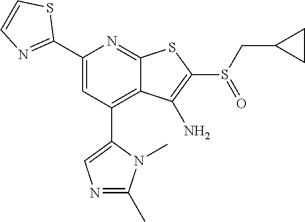 [SMILES: Cc1ncc(-c2cc(-c3nccs3)nc3sc(S(=O)CC4CC4)c(N)c23)n1C] | ** |
| 4 | 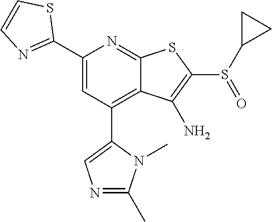 [SMILES: Cc1ncc(-c2cc(-c3nccs3)nc3sc(S(=O)C4CC4)c(N)c23)n1C] | ** |
| 5 | 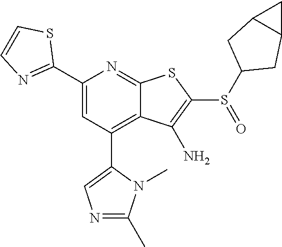 [SMILES: Cc1ncc(-c2cc(-c3nccs3)nc3sc(S(=O)C4CC5CC5C4)c(N)c23)n1C] | *** |
| 6 | 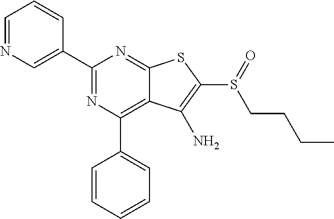 [SMILES: CCCCS(=O)c1sc2nc(-c3cccnc3)nc(-c3ccccc3)c2c1N] | *** |
| 7 | 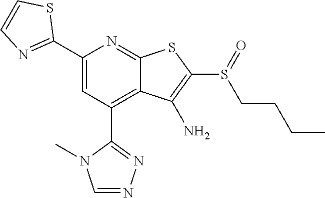 [SMILES: CCCCS(=O)c1sc2nc(-c3nccs3)cc(-c3nncn3C)c2c1N] | *** |
| 8 | 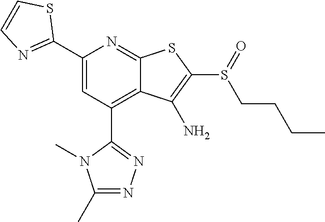 [SMILES: CCCCS(=O)c1sc2nc(-c3nccs3)cc(-c3nnc(C)n3C)c2c1N] | *** |
| 9 | 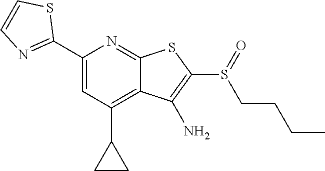 [SMILES: CCCCS(=O)c1sc2nc(-c3nccs3)cc(C3CC3)c2c1N] | *** |
| 10 | 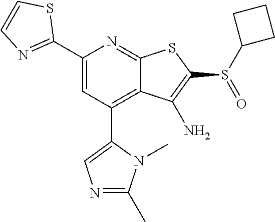 [SMILES: Cc1ncc(-c2cc(-c3nccs3)nc3sc([S@](=O)C4CCC4)c(N)c23)n1C] | *** |
| 11 | 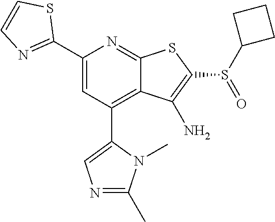 [SMILES: Cc1ncc(-c2cc(-c3nccs3)nc3sc([S@@](=O)C4CCC4)c(N)c23)n1C] | * |
| 12 | 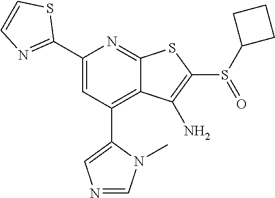 [SMILES: Cn1cncc1-c1cc(-c2nccs2)nc2sc(S(=O)C3CCC3)c(N)c12] | *** |
| 13 | 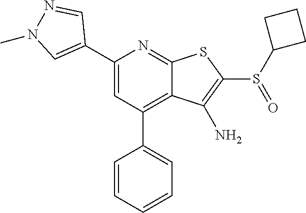 [SMILES: Cn1cc(-c2cc(-c3ccccc3)c3c(N)c(S(=O)C4CCC4)sc3n2)cn1] | *** |
| 14 | 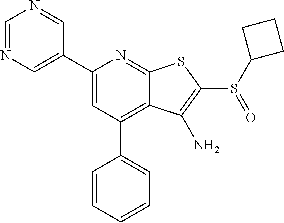 [SMILES: Nc1c(S(=O)C2CCC2)sc2nc(-c3cncnc3)cc(-c3ccccc3)c12] | *** |
| 15 | 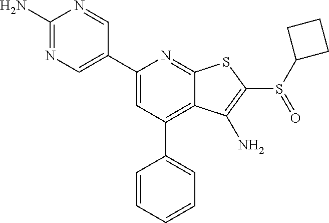 [SMILES: Nc1ncc(-c2cc(-c3ccccc3)c3c(N)c(S(=O)C4CCC4)sc3n2)cn1] | *** |
| 16 | 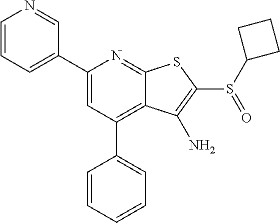 [SMILES: Nc1c(S(=O)C2CCC2)sc2nc(-c3cccnc3)cc(-c3ccccc3)c12] | *** |
| 17 | 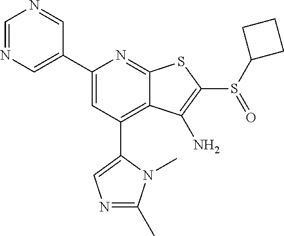 [SMILES: Cc1ncc(-c2cc(-c3cncnc3)nc3sc(S(=O)C4CCC4)c(N)c23)n1C] | *** |
| 18 | 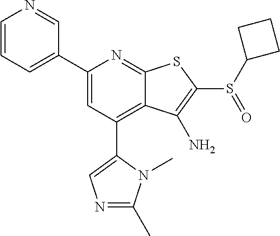 [SMILES: Cc1ncc(-c2cc(-c3cccnc3)nc3sc(S(=O)C4CCC4)c(N)c23)n1C] | *** |
| 19 | 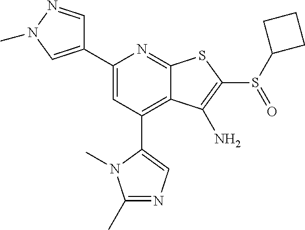 [SMILES: Cc1ncc(-c2cc(-c3cnn(C)c3)nc3sc(S(=O)C4CCC4)c(N)c23)n1C] | *** |
| 20 | 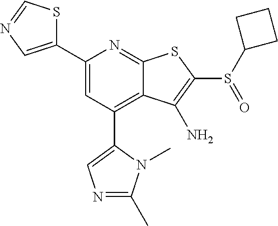 [SMILES: Cc1ncc(-c2cc(-c3cncs3)nc3sc(S(=O)C4CCC4)c(N)c23)n1C] | *** |
| 21 | 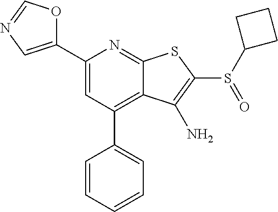 [SMILES: Nc1c(S(=O)C2CCC2)sc2nc(-c3cnco3)cc(-c3ccccc3)c12] | *** |
| 22 | 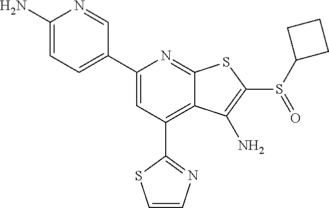 [SMILES: Nc1ccc(-c2cc(-c3nccs3)c3c(N)c(S(=O)C4CCC4)sc3n2)cn1] | *** |
| 23 | 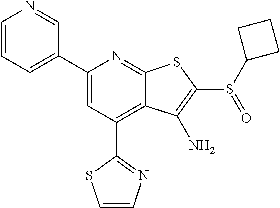 [SMILES: Nc1c(S(=O)C2CCC2)sc2nc(-c3cccnc3)cc(-c3nccs3)c12] | *** |
| 24 | 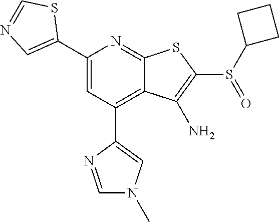 [SMILES: Cn1cnc(-c2cc(-c3cncs3)nc3sc(S(=O)C4CCC4)c(N)c23)c1] | *** |
| 25 | 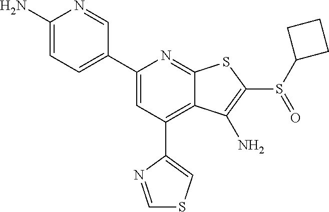 [SMILES: Nc1ccc(-c2cc(-c3cscn3)c3c(N)c(S(=O)C4CCC4)sc3n2)cn1] | *** |
| 26 | 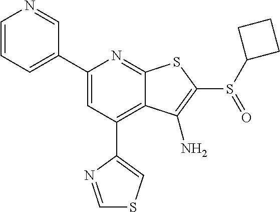 [SMILES: Nc1c(S(=O)C2CCC2)sc2nc(-c3cccnc3)cc(-c3cscn3)c12] | *** |
| 27 | 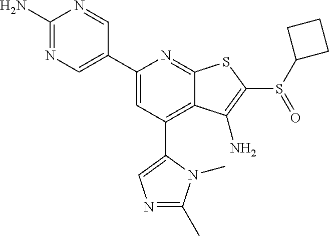 [SMILES: Cc1ncc(-c2cc(-c3cnc(N)nc3)nc3sc(S(=O)C4CCC4)c(N)c23)n1C] | *** |
| 28 | 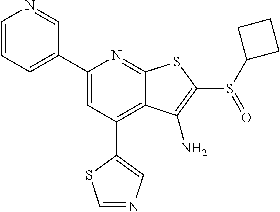 [SMILES: Nc1c(S(=O)C2CCC2)sc2nc(-c3cccnc3)cc(-c3cncs3)c12] | *** |
| 29 | 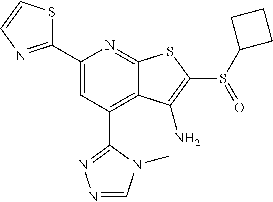 [SMILES: Cn1cnnc1-c1cc(-c2nccs2)nc2sc(S(=O)C3CCC3)c(N)c12] | *** |
| 30 | 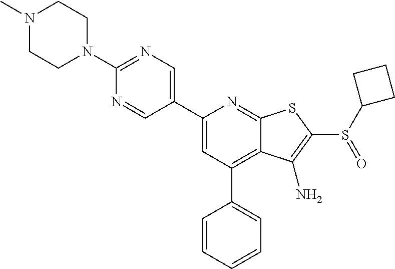 [SMILES: CN1CCN(c2ncc(-c3cc(-c4ccccc4)c4c(N)c(S(=O)C5CCC5)sc4n3)cn2)CC1] | *** |
| 31 | 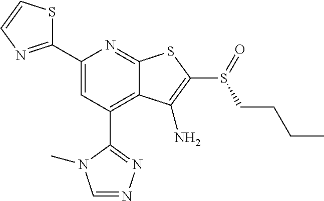 [SMILES: CCCC[S@@](=O)c1sc2nc(-c3nccs3)cc(-c3nncn3C)c2c1N] | *** |
| 32 | 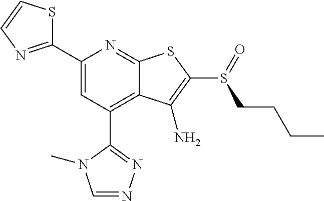 [SMILES: CCCC[S@](=O)c1sc2nc(-c3nccs3)cc(-c3nncn3C)c2c1N] | * |
| 33 | 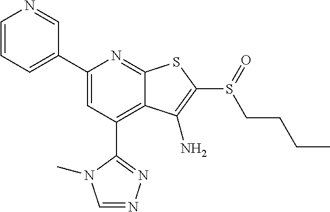 [SMILES: CCCCS(=O)c1sc2nc(-c3cccnc3)cc(-c3nncn3C)c2c1N] | *** |
| 34 | 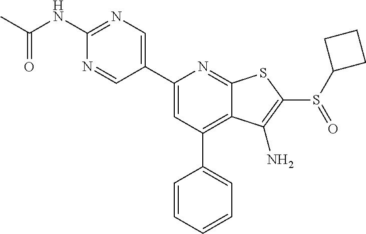 [SMILES: CC(=O)Nc1ncc(-c2cc(-c3ccccc3)c3c(N)c(S(=O)C4CCC4)sc3n2)cn1] | *** |
| 35 | 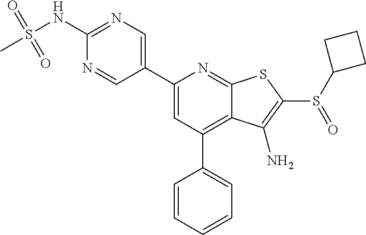 [SMILES: CS(=O)(=O)Nc1ncc(-c2cc(-c3ccccc3)c3c(N)c(S(=O)C4CCC4)sc3n2)cn1] | *** |
| 36 | 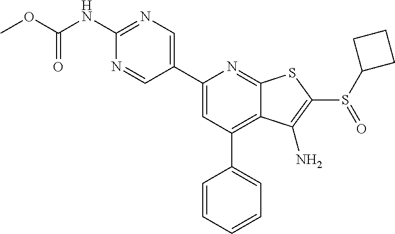 [SMILES: COC(=O)Nc1ncc(-c2cc(-c3ccccc3)c3c(N)c(S(=O)C4CCC4)sc3n2)cn1] | *** |
| 37 | 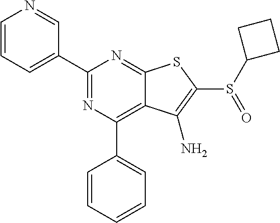 [SMILES: Nc1c(S(=O)C2CCC2)sc2nc(-c3cccnc3)nc(-c3ccccc3)c12] | *** |
| 38 | 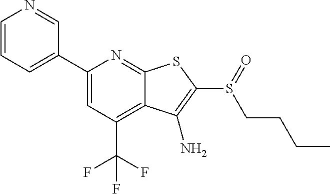 [SMILES: CCCCS(=O)c1sc2nc(-c3cccnc3)cc(C(F)(F)F)c2c1N] | *** |
| 39 | 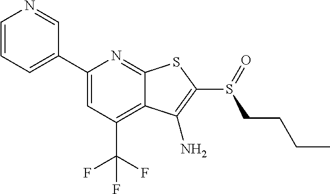 [SMILES: CCCC[S@](=O)c1sc2nc(-c3cccnc3)cc(C(F)(F)F)c2c1N] | *** |
| 40 | 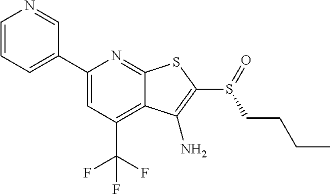 [SMILES: CCCC[S@@](=O)c1sc2nc(-c3cccnc3)cc(C(F)(F)F)c2c1N] | *** |
| 41 | 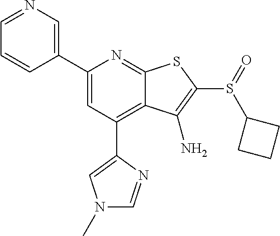 [SMILES: Cn1cnc(-c2cc(-c3cccnc3)nc3sc(S(=O)C4CCC4)c(N)c23)c1] | *** |
| 42 | 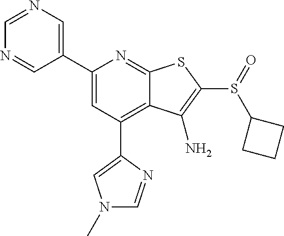 [SMILES: Cn1cnc(-c2cc(-c3cncnc3)nc3sc(S(=O)C4CCC4)c(N)c23)c1] | *** |
| 43 | 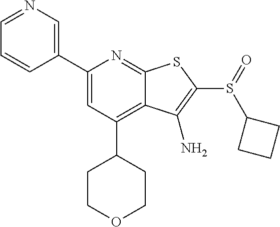 [SMILES: Nc1c(S(=O)C2CCC2)sc2nc(-c3cccnc3)cc(C3CCOCC3)c12] | *** |
| 44 | 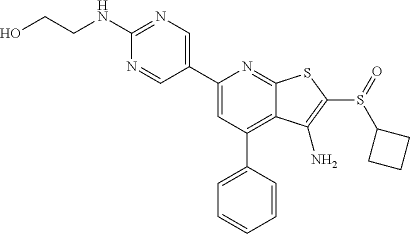 [SMILES: Nc1c(S(=O)C2CCC2)sc2nc(-c3cnc(NCCO)nc3)cc(-c3ccccc3)c12] | *** |
| 45 | 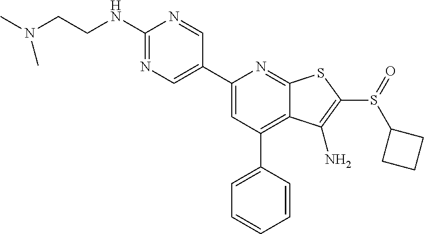 [SMILES: CN(C)CCNc1ncc(-c2cc(-c3ccccc3)c3c(N)c(S(=O)C4CCC4)sc3n2)cn1] | * |
| 46 | 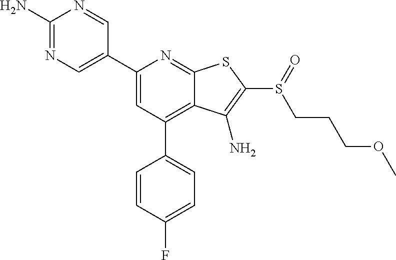 [SMILES: COCCCS(=O)c1sc2nc(-c3cnc(N)nc3)cc(-c3ccc(F)cc3)c2c1N] | *** |
| 47 | 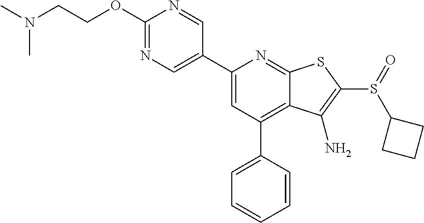 [SMILES: CN(C)CCOc1ncc(-c2cc(-c3ccccc3)c3c(N)c(S(=O)C4CCC4)sc3n2)cn1] | ** |
| 48 | 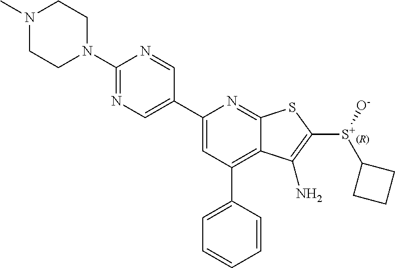 | *** |
| 49 | 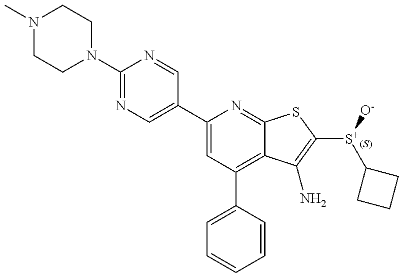 | * |
| 50 | 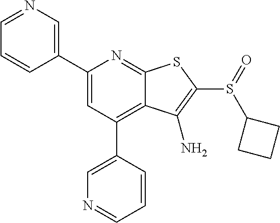 [SMILES: Nc1c(S(=O)C2CCC2)sc2nc(-c3cccnc3)cc(-c3cccnc3)c12] | *** |
| 51 | 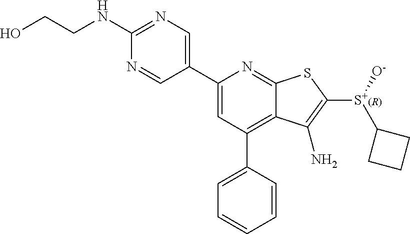 | *** |
| 52 | 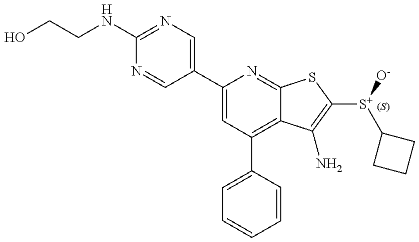 | * |
| 53 | 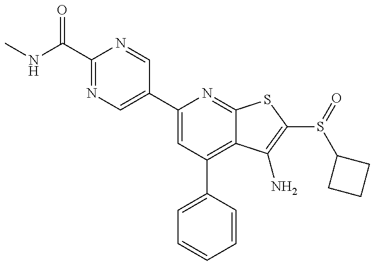 [SMILES: CNC(=O)c1ncc(-c2cc(-c3ccccc3)c3c(N)c(S(=O)C4CCC4)sc3n2)cn1] | *** |
| 54 | 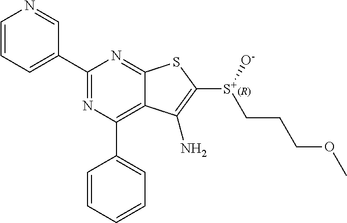 | *** |
| 55 | 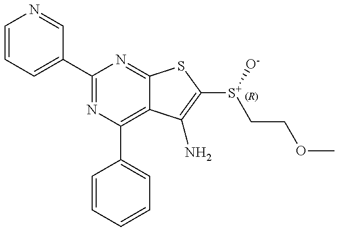 | *** |
| 56 | 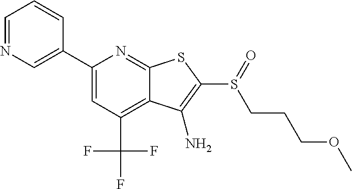 [SMILES: COCCCS(=O)c1sc2nc(-c3cccnc3)cc(C(F)(F)F)c2c1N] | *** |
| 57 | 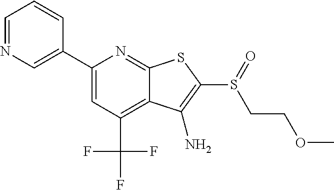 [SMILES: COCCS(=O)c1sc2nc(-c3cccnc3)cc(C(F)(F)F)c2c1N] | *** |
| 58 | 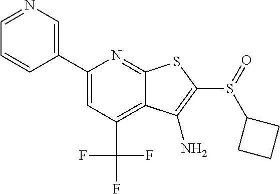 [SMILES: Nc1c(S(=O)C2CCC2)sc2nc(-c3cccnc3)cc(C(F)(F)F)c12] | *** |
| 59 | 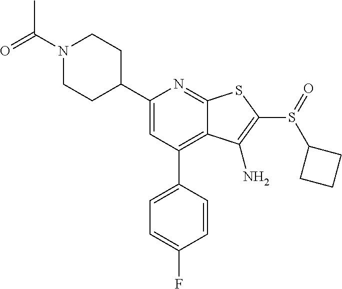 [SMILES: CC(=O)N1CCC(c2cc(-c3ccc(F)cc3)c3c(N)c(S(=O)C4CCC4)sc3n2)CC1] | * |
| 60 | 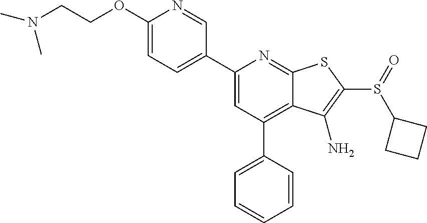 [SMILES: CN(C)CCOc1ccc(-c2cc(-c3ccccc3)c3c(N)c(S(=O)C4CCC4)sc3n2)cn1] | ** |
| 61 | 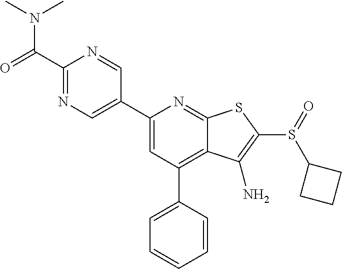 [SMILES: CN(C)C(=O)c1ncc(-c2cc(-c3ccccc3)c3c(N)c(S(=O)C4CCC4)sc3n2)cn1] | ** |
| 62 | 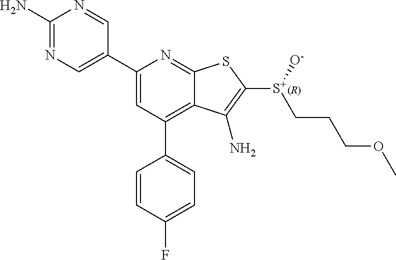 | *** |
| 63 | 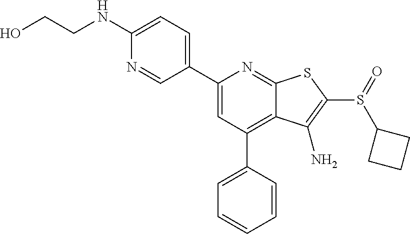 [SMILES: Nc1c(S(=O)C2CCC2)sc2nc(-c3ccc(NCCO)nc3)cc(-c3ccccc3)c12] | *** |
| 64 | 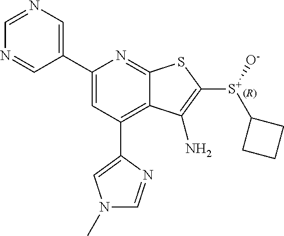 | *** |
| 65 | 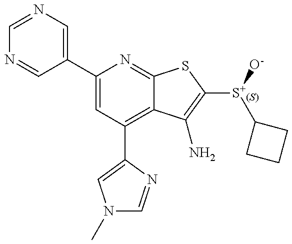 | * |
| 66 | 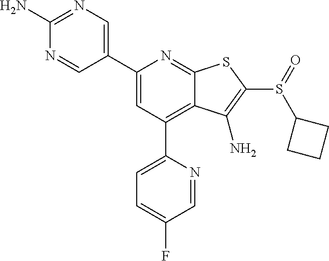 [SMILES: Nc1ncc(-c2cc(-c3ccc(F)cn3)c3c(N)c(S(=O)C4CCC4)sc3n2)cn1] | *** |
| 67 | 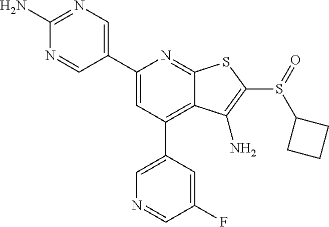 [SMILES: Nc1ncc(-c2cc(-c3cncc(F)c3)c3c(N)c(S(=O)C4CCC4)sc3n2)cn1] | *** |
| 68 | 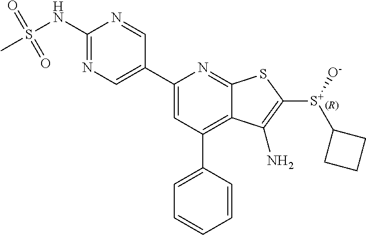 | *** |
| 69 | 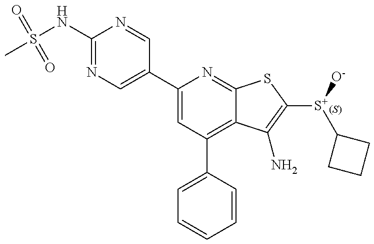 | * |
| 70 | 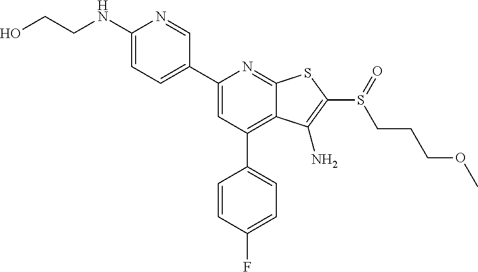 [SMILES: COCCCS(=O)c1sc2nc(-c3ccc(NCCO)nc3)cc(-c3ccc(F)cc3)c2c1N] | ** |
| 71 | 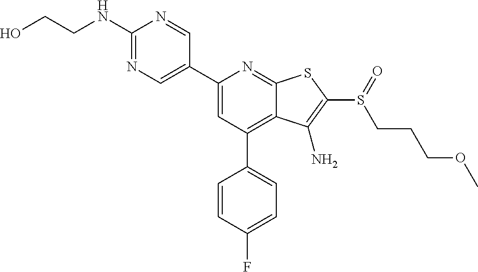 [SMILES: COCCCS(=O)c1sc2nc(-c3cnc(NCCO)nc3)cc(-c3ccc(F)cc3)c2c1N] | ** |
| 72 | 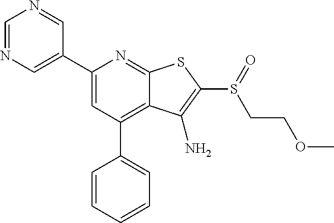 [SMILES: COCCS(=O)c1sc2nc(-c3cncnc3)cc(-c3ccccc3)c2c1N] | *** |
| 73 | 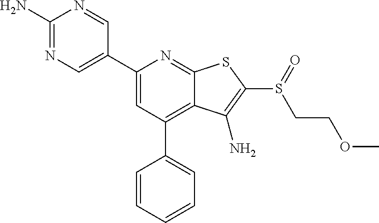 [SMILES: COCCS(=O)c1sc2nc(-c3cnc(N)nc3)cc(-c3ccccc3)c2c1N] | *** |
| 74 | 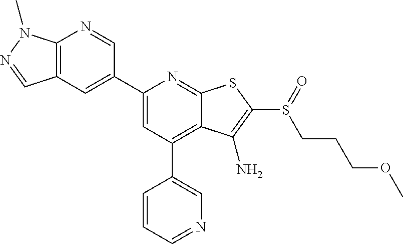 [SMILES: COCCCS(=O)c1sc2nc(-c3cnc4c(cnn4C)c3)cc(-c3cccnc3)c2c1N] | *** |
| 75 | 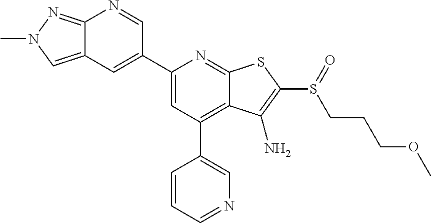 | *** |
| 76 | 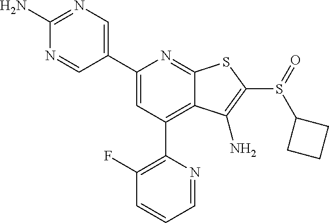 [SMILES: Nc1ncc(-c2cc(-c3ncccc3F)c3c(N)c(S(=O)C4CCC4)sc3n2)cn1] | *** |
| 77 | 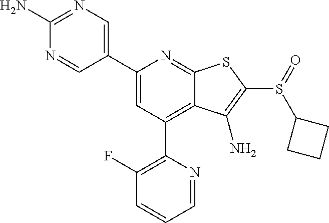 [SMILES: Nc1ncc(-c2cc(-c3ncccc3F)c3c(N)c(S(=O)C4CCC4)sc3n2)cn1] | *** |
| 78 | 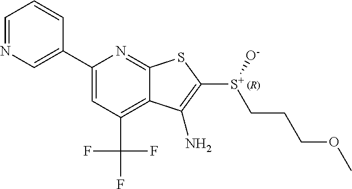 | *** |
| 79 | 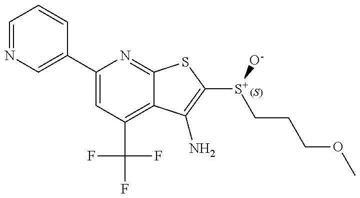 | * |
| 80 | 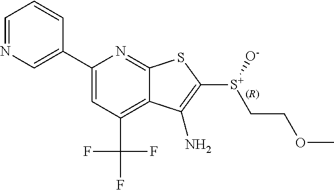 | *** |
| 81 | 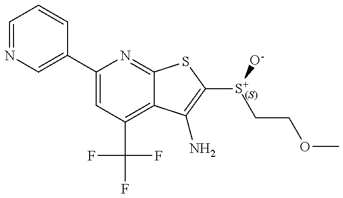 | * |
| 82 | 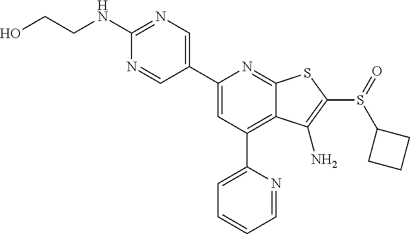 [SMILES: Nc1c(S(=O)C2CCC2)sc2nc(-c3cnc(NCCO)nc3)cc(-c3ccccn3)c12] | *** |
| 83 | 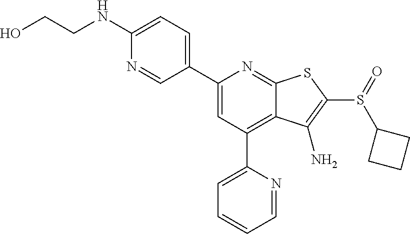 [SMILES: Nc1c(S(=O)C2CCC2)sc2nc(-c3ccc(NCCO)nc3)cc(-c3ccccn3)c12] | *** |
| 84 | 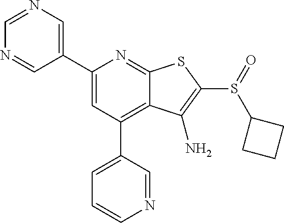 [SMILES: Nc1c(S(=O)C2CCC2)sc2nc(-c3cncnc3)cc(-c3cccnc3)c12] | *** |
| 85 | 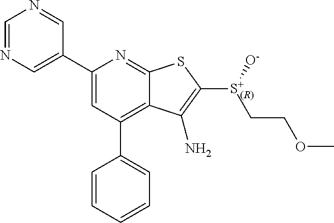 | *** |
| 86 | 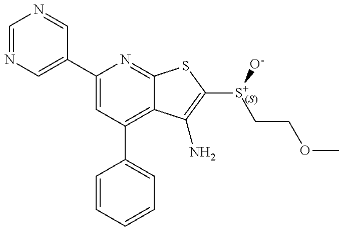 | * |
| 87 | 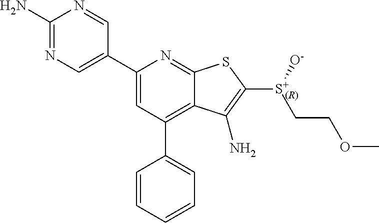 | *** |
| 88 | 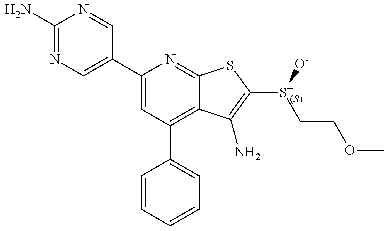 | * |
| 89 | 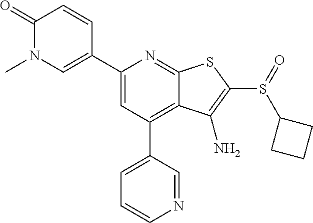 [SMILES: Cn1cc(-c2cc(-c3cccnc3)c3c(N)c(S(=O)C4CCC4)sc3n2)ccc1=O] | *** |
| 90 | 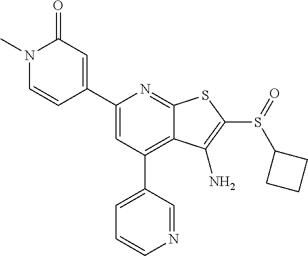 [SMILES: Cn1ccc(-c2cc(-c3cccnc3)c3c(N)c(S(=O)C4CCC4)sc3n2)cc1=O] | ** |
| 91 | 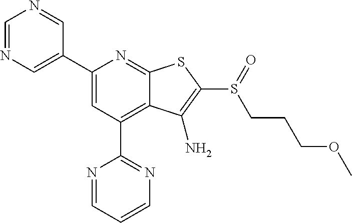 [SMILES: COCCCS(=O)c1sc2nc(-c3cncnc3)cc(-c3ncccn3)c2c1N] | *** |
| 92 | 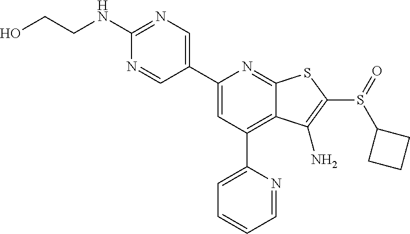 [SMILES: Nc1c(S(=O)C2CCC2)sc2nc(-c3cnc(NCCO)nc3)cc(-c3ccccn3)c12] | *** |
| 93 | 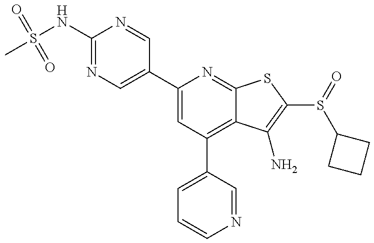 [SMILES: CS(=O)(=O)Nc1ncc(-c2cc(-c3cccnc3)c3c(N)c(S(=O)C4CCC4)sc3n2)cn1] | *** |
| 94 | 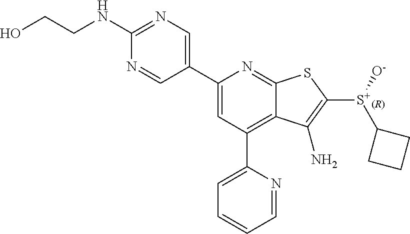 | *** |
| 95 | 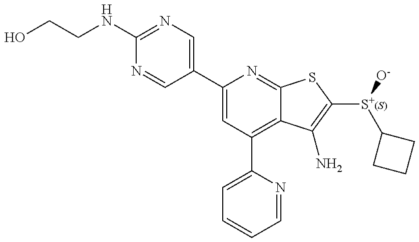 | * |
| 96 | 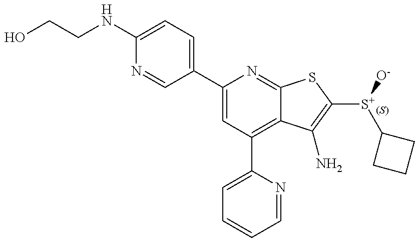 | *** |
| 97 | 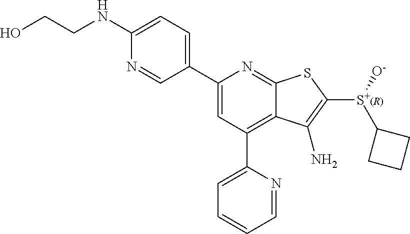 | *** |
| 98 | 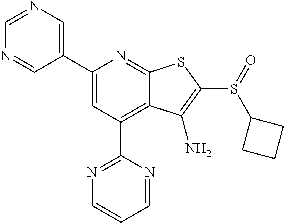 [SMILES: Nc1c(S(=O)C2CCC2)sc2nc(-c3cncnc3)cc(-c3ncccn3)c12] | *** |
| 99 | 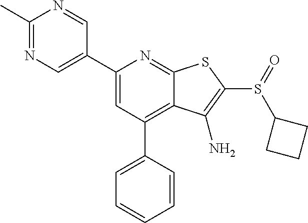 [SMILES: Cc1ncc(-c2cc(-c3ccccc3)c3c(N)c(S(=O)C4CCC4)sc3n2)cn1] | *** |
| 100 | 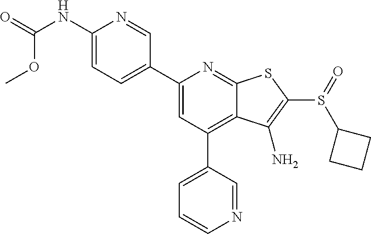 [SMILES: COC(=O)Nc1ccc(-c2cc(-c3cccnc3)c3c(N)c(S(=O)C4CCC4)sc3n2)cn1] | *** |
| 101 | 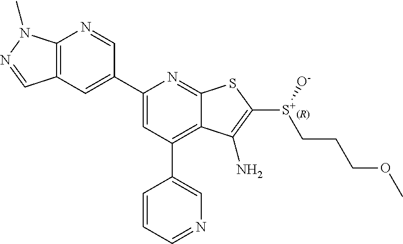 | *** |
| 102 | 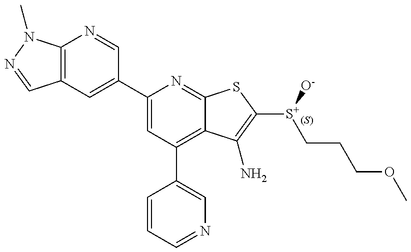 | * |
| 103 | 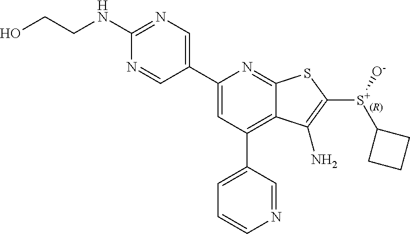 | *** |
| 104 | 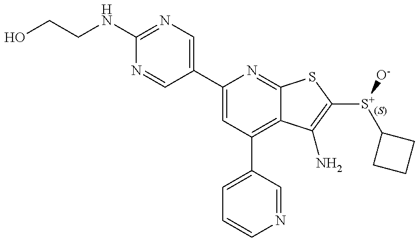 | * |
| 105 | 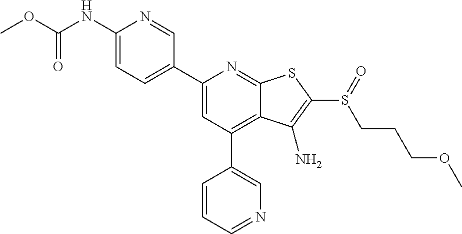 | ** |
| 106 | 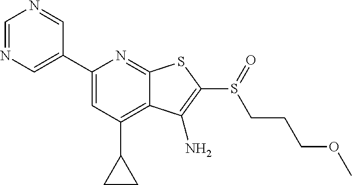 [SMILES: COCCCS(=O)c1sc2nc(-c3cncnc3)cc(C3CC3)c2c1N] | *** |
| 107 | 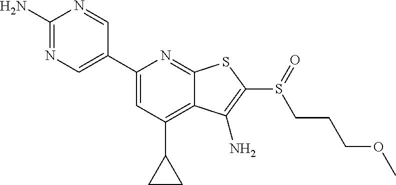 [SMILES: COCCCS(=O)c1sc2nc(-c3cnc(N)nc3)cc(C3CC3)c2c1N] | *** |
| 108 | 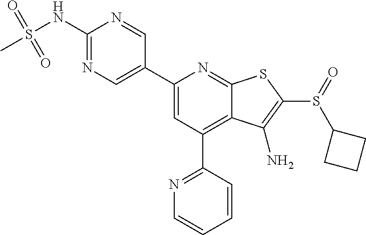 [SMILES: CS(=O)(=O)Nc1ncc(-c2cc(-c3ccccn3)c3c(N)c(S(=O)C4CCC4)sc3n2)cn1] | *** |
| 109 | 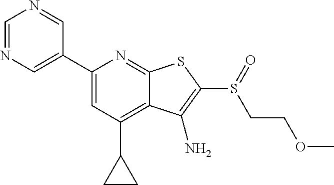 [SMILES: COCCS(=O)c1sc2nc(-c3cncnc3)cc(C3CC3)c2c1N] | *** |
| 110 | 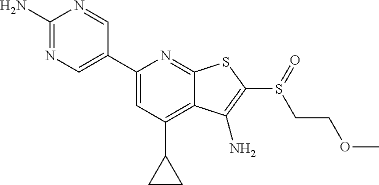 [SMILES: COCCS(=O)c1sc2nc(-c3cnc(N)nc3)cc(C3CC3)c2c1N] | *** |
| 111 | 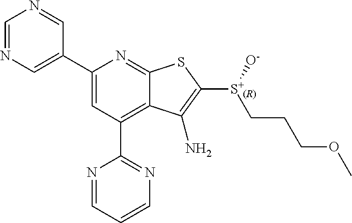 | *** |
| 112 | 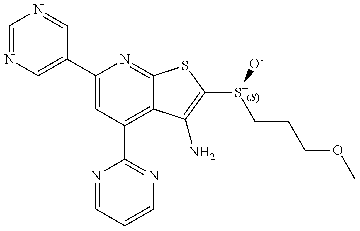 | * |
| 113 | 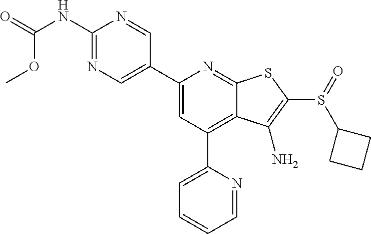 [SMILES: COC(=O)Nc1ncc(-c2cc(-c3ccccn3)c3c(N)c(S(=O)C4CCC4)sc3n2)cn1] | ** |
| 114 | 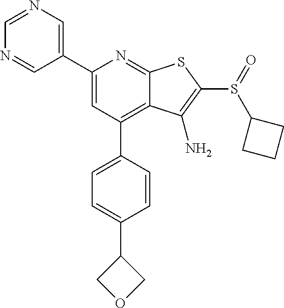 [SMILES: Nc1c(S(=O)C2CCC2)sc2nc(-c3cncnc3)cc(-c3ccc(C4COC4)cc3)c12] | *** |
| 115 | 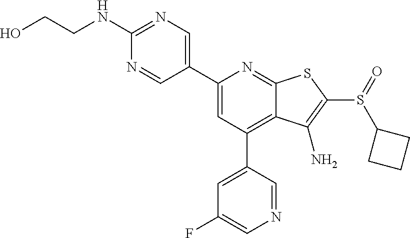 [SMILES: Nc1c(S(=O)C2CCC2)sc2nc(-c3cnc(NCCO)nc3)cc(-c3cncc(F)c3)c12] | *** |
| 116 | 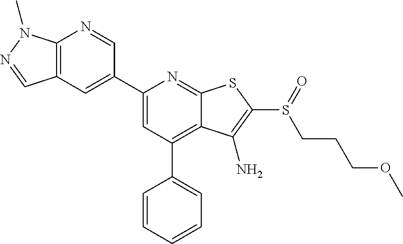 [SMILES: COCCCS(=O)c1sc2nc(-c3cnc4c(cnn4C)c3)cc(-c3ccccc3)c2c1N] | ** |
| 117 | 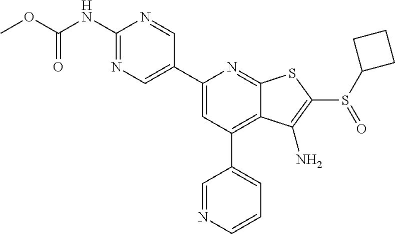 [SMILES: COC(=O)Nc1ncc(-c2cc(-c3cccnc3)c3c(N)c(S(=O)C4CCC4)sc3n2)cn1] | ** |
| 118 | 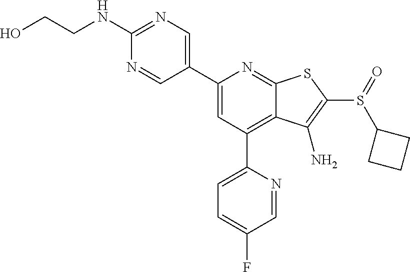 [SMILES: Nc1c(S(=O)C2CCC2)sc2nc(-c3cnc(NCCO)nc3)cc(-c3ccc(F)cn3)c12] | ** |
| 119 | 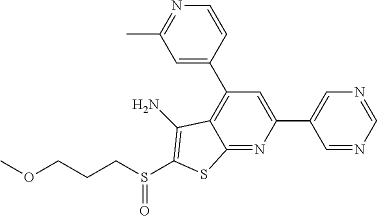 [SMILES: COCCCS(=O)c1sc2nc(-c3cncnc3)cc(-c3ccnc(C)c3)c2c1N] | *** |
| 120 | 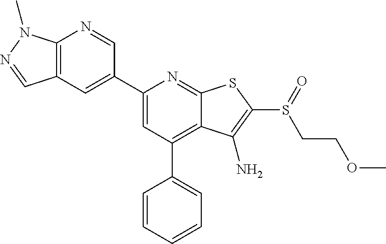 [SMILES: COCCS(=O)c1sc2nc(-c3cnc4c(cnn4C)c3)cc(-c3ccccc3)c2c1N] | *** |
| 121 | 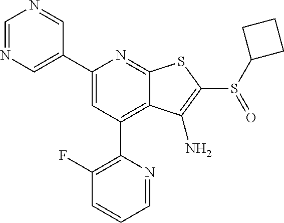 [SMILES: Nc1c(S(=O)C2CCC2)sc2nc(-c3cncnc3)cc(-c3ncccc3F)c12] | *** |
| 122 | 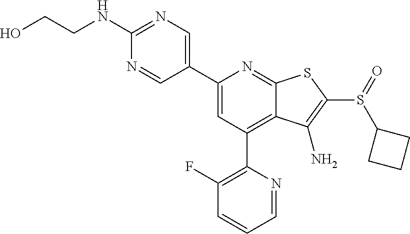 [SMILES: Nc1c(S(=O)C2CCC2)sc2nc(-c3cnc(NCCO)nc3)cc(-c3ncccc3F)c12] | *** |
| 123 | 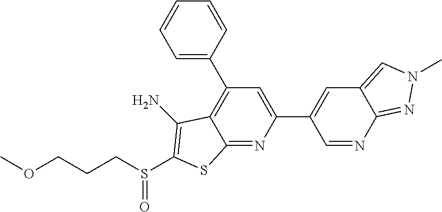 | *** |
| 124 | 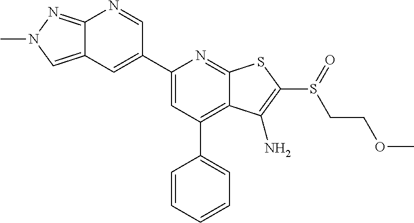 | *** |
| 125 | 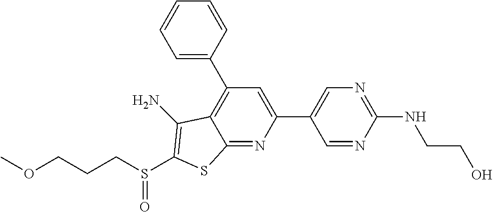 [SMILES: COCCCS(=O)c1sc2nc(-c3cnc(NCCO)nc3)cc(-c3ccccc3)c2c1N] | *** |
| 126 | 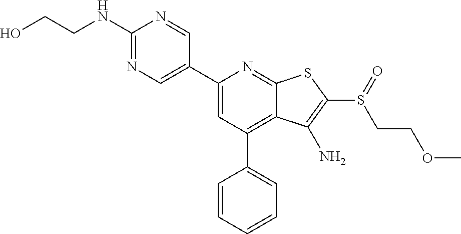 [SMILES: COCCS(=O)c1sc2nc(-c3cnc(NCCO)nc3)cc(-c3ccccc3)c2c1N] | *** |
| 127 | 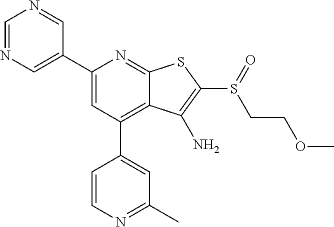 [SMILES: COCCS(=O)c1sc2nc(-c3cncnc3)cc(-c3ccnc(C)c3)c2c1N] | *** |
| 128 | 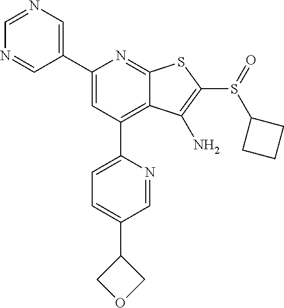 [SMILES: Nc1c(S(=O)C2CCC2)sc2nc(-c3cncnc3)cc(-c3ccc(C4COC4)cn3)c12] | *** |
| 129 | 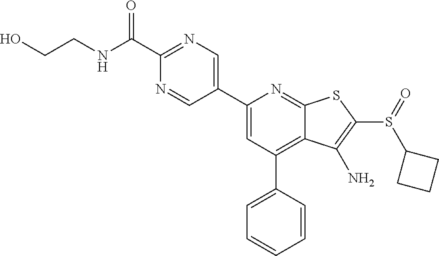 [SMILES: Nc1c(S(=O)C2CCC2)sc2nc(-c3cnc(C(=O)NCCO)nc3)cc(-c3ccccc3)c12] | *** |
| 130 | 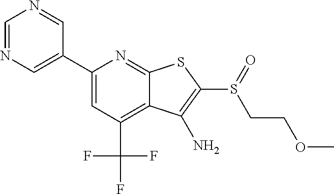 [SMILES: COCCS(=O)c1sc2nc(-c3cncnc3)cc(C(F)(F)F)c2c1N] | *** |
| 131 | 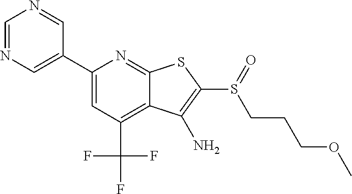 [SMILES: COCCCS(=O)c1sc2nc(-c3cncnc3)cc(C(F)(F)F)c2c1N] | *** |
| 132 | 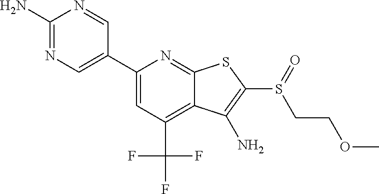 [SMILES: COCCS(=O)c1sc2nc(-c3cnc(N)nc3)cc(C(F)(F)F)c2c1N] | *** |
| 133 | 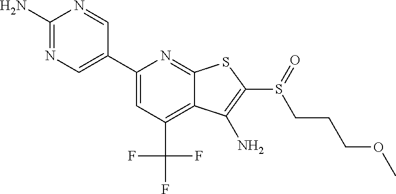 [SMILES: COCCCS(=O)c1sc2nc(-c3cnc(N)nc3)cc(C(F)(F)F)c2c1N] | *** |
| 134 | 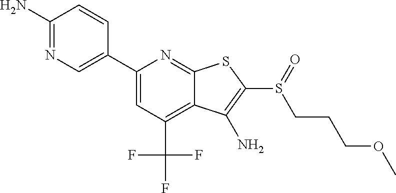 [SMILES: COCCCS(=O)c1sc2nc(-c3ccc(N)nc3)cc(C(F)(F)F)c2c1N] | ** |
| 135 | 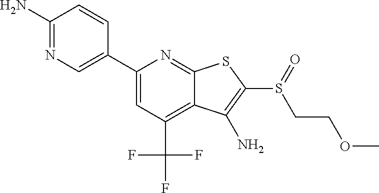 [SMILES: COCCS(=O)c1sc2nc(-c3ccc(N)nc3)cc(C(F)(F)F)c2c1N] | ** |
| 136 | 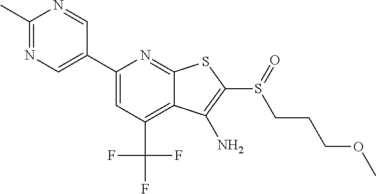 | ** |
| 137 | 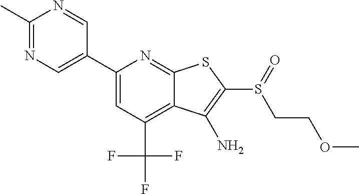 | *** |
| 138 | 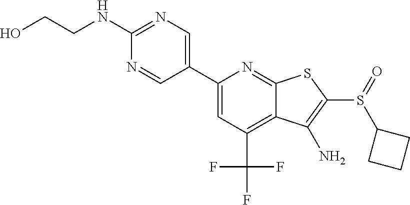 [SMILES: Nc1c(S(=O)C2CCC2)sc2nc(-c3cnc(NCCO)nc3)cc(C(F)(F)F)c12] | ** |
| 139 | 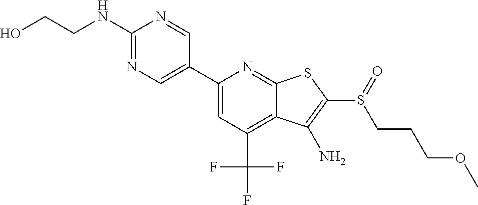 [SMILES: COCCCS(=O)c1sc2nc(-c3cnc(NCCO)nc3)cc(C(F)(F)F)c2c1N] | ** |
| 140 | 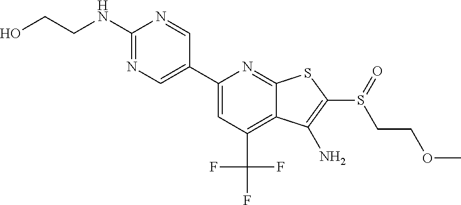 [SMILES: COCCS(=O)c1sc2nc(-c3cnc(NCCO)nc3)cc(C(F)(F)F)c2c1N] | ** |
| 141 | 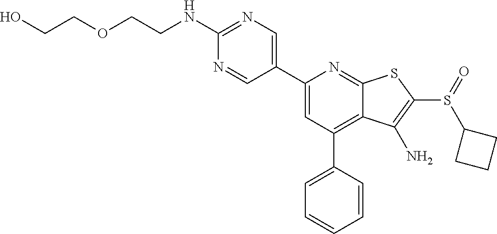 [SMILES: Nc1c(S(=O)C2CCC2)sc2nc(-c3cnc(NCCOCCO)nc3)cc(-c3ccccc3)c12] | *** |
| 142 | 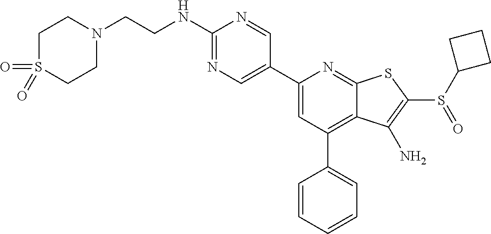 [SMILES: Nc1c(S(=O)C2CCC2)sc2nc(-c3cnc(NCCN4CCS(=O)(=O)CC4)nc3)cc(-c3ccccc3)c12] | *** |
| 143 | 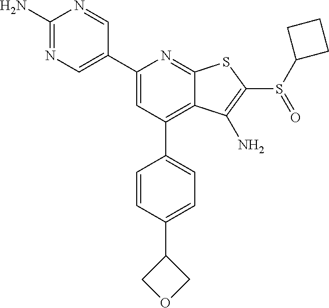 [SMILES: Nc1ncc(-c2cc(-c3ccc(C4COC4)cc3)c3c(N)c(S(=O)C4CCC4)sc3n2)cn1] | *** |
| 144 | 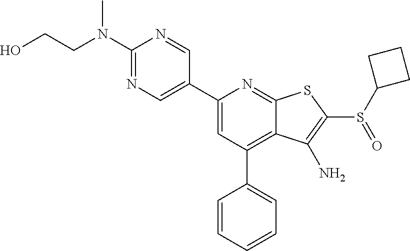 [SMILES: CN(CCO)c1ncc(-c2cc(-c3ccccc3)c3c(N)c(S(=O)C4CCC4)sc3n2)cn1] | *** |
| 145 | 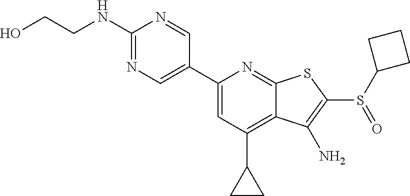 [SMILES: Nc1c(S(=O)C2CCC2)sc2nc(-c3cnc(NCCO)nc3)cc(C3CC3)c12] | *** |
| 146 | 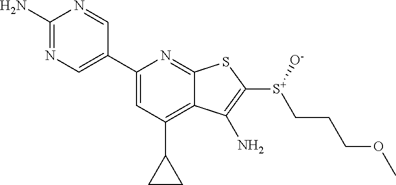 [SMILES: COCCC[S@@+]([O-])c1sc2nc(-c3cnc(N)nc3)cc(C3CC3)c2c1N] | *** |
| 147 | 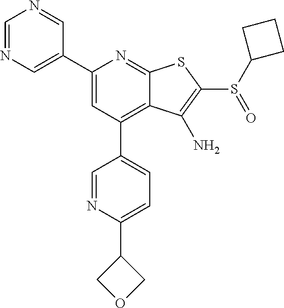 [SMILES: Nc1c(S(=O)C2CCC2)sc2nc(-c3cncnc3)cc(-c3ccc(C4COC4)nc3)c12] | *** |
| 148 | 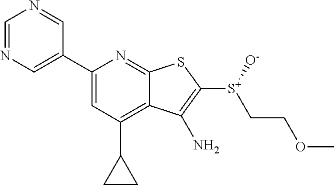 [SMILES: COCC[S@@+]([O-])c1sc2nc(-c3cncnc3)cc(C3CC3)c2c1N] | *** |
| 149 | 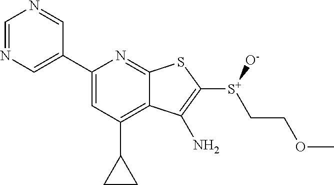 [SMILES: COCC[S@+]([O-])c1sc2nc(-c3cncnc3)cc(C3CC3)c2c1N] | * |
| 150 | 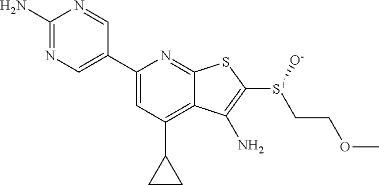 [SMILES: COCC[S@@+]([O-])c1sc2nc(-c3cnc(N)nc3)cc(C3CC3)c2c1N] | *** |
| 151 | 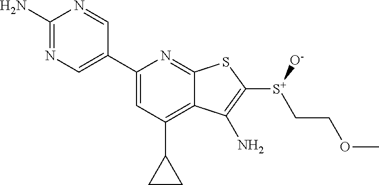 [SMILES: COCC[S@+]([O-])c1sc2nc(-c3cnc(N)nc3)cc(C3CC3)c2c1N] | * |
| 152 | 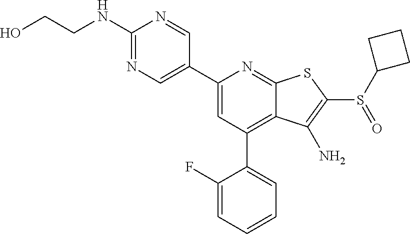 [SMILES: Nc1c(S(=O)C2CCC2)sc2nc(-c3cnc(NCCO)nc3)cc(-c3ccccc3F)c12] | *** |
| 153 | 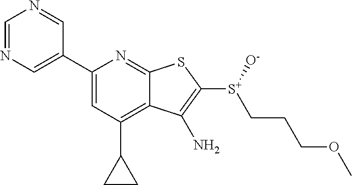 [SMILES: COCCC[S@@+]([O-])c1sc2nc(-c3cncnc3)cc(C3CC3)c2c1N] | *** |
| 154 | 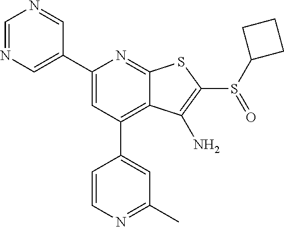 [SMILES: Cc1cc(-c2cc(-c3cncnc3)nc3sc(S(=O)C4CCC4)c(N)c23)ccn1] | *** |
| 155 | 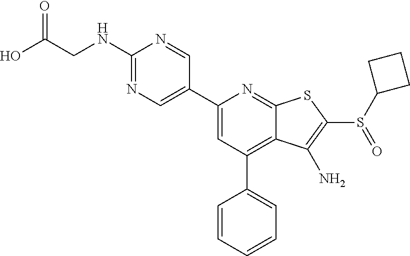 [SMILES: Nc1c(S(=O)C2CCC2)sc2nc(-c3cnc(NCC(=O)O)nc3)cc(-c3ccccc3)c12] | *** |
| 156 | 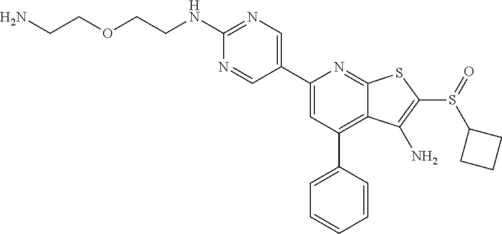 [SMILES: NCCOCCNc1ncc(-c2cc(-c3ccccc3)c3c(N)c(S(=O)C4CCC4)sc3n2)cn1] | *** |
| 157 | 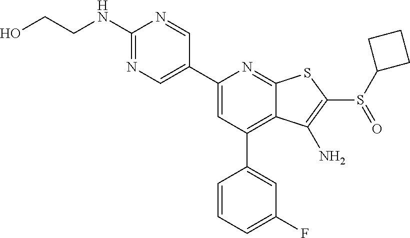 [SMILES: Nc1c(S(=O)C2CCC2)sc2nc(-c3cnc(NCCO)nc3)cc(-c3cccc(F)c3)c12] | *** |
| 158 | 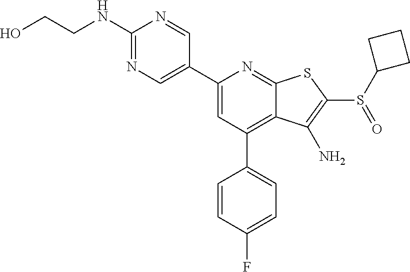 [SMILES: Nc1c(S(=O)C2CCC2)sc2nc(-c3cnc(NCCO)nc3)cc(-c3ccc(F)cc3)c12] | ** |
| 159 | 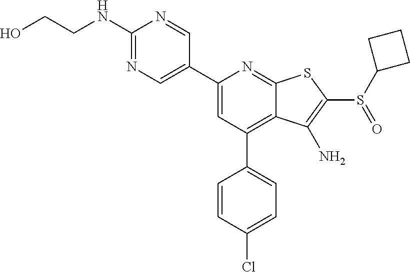 [SMILES: Nc1c(S(=O)C2CCC2)sc2nc(-c3cnc(NCCO)nc3)cc(-c3ccc(Cl)cc3)c12] | *** |
| 160 | 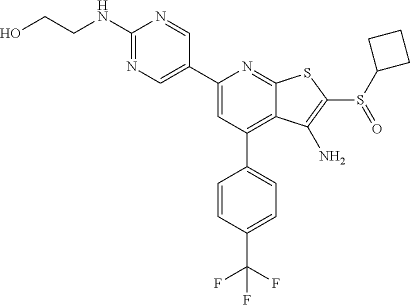 [SMILES: Nc1c(S(=O)C2CCC2)sc2nc(-c3cnc(NCCO)nc3)cc(-c3ccc(C(F)(F)F)cc3)c12] | ** |
| 161 | 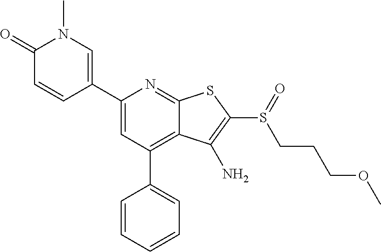 [SMILES: COCCCS(=O)c1sc2nc(-c3ccc(=O)n(C)c3)cc(-c3ccccc3)c2c1N] | *** |
| 162 | 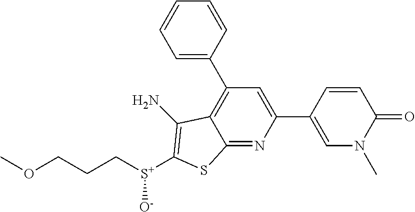 [SMILES: COCCC[S@@+]([O-])c1sc2nc(-c3ccc(=O)n(C)c3)cc(-c3ccccc3)c2c1N] | *** |
| 163 | 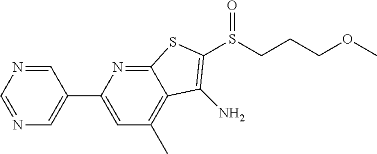 [SMILES: COCCCS(=O)c1sc2nc(-c3cncnc3)cc(C)c2c1N] | *** |
| 164 | 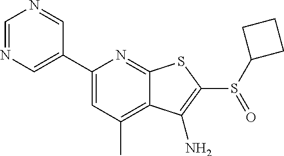 [SMILES: Cc1cc(-c2cncnc2)nc2sc(S(=O)C3CCC3)c(N)c12] | *** |
| 165 | 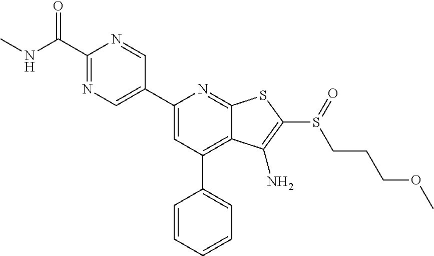 | ** |
| 166 | 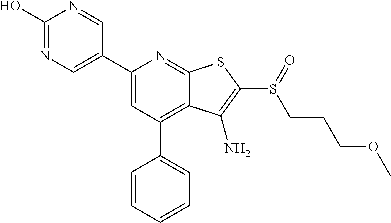 [SMILES: COCCCS(=O)c1sc2nc(-c3cnc(O)nc3)cc(-c3ccccc3)c2c1N] | ** |
| 167 | 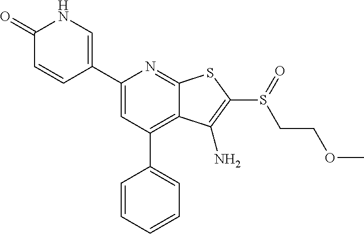 [SMILES: COCCS(=O)c1sc2nc(-c3ccc(=O)[nH]c3)cc(-c3ccccc3)c2c1N] | *** |
| 168 | 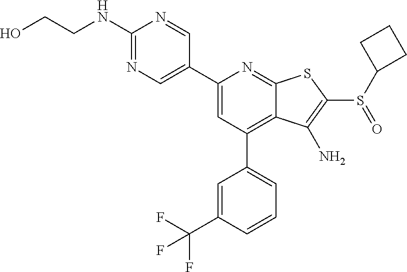 [SMILES: Nc1c(S(=O)C2CCC2)sc2nc(-c3cnc(NCCO)nc3)cc(-c3cccc(C(F)(F)F)c3)c12] | ** |
| 169 | 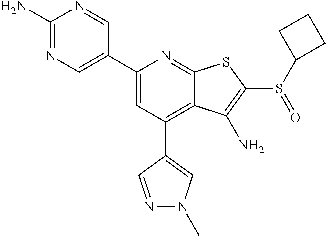 [SMILES: Cn1cc(-c2cc(-c3cnc(N)nc3)nc3sc(S(=O)C4CCC4)c(N)c23)cn1] | *** |
| 170 | 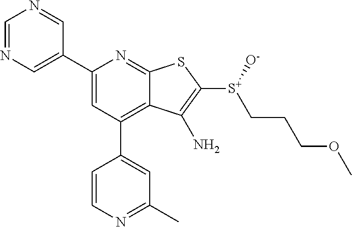 [SMILES: COCCC[S@@+]([O-])c1sc2nc(-c3cncnc3)cc(-c3ccnc(C)c3)c2c1N] | *** |
| 171 | 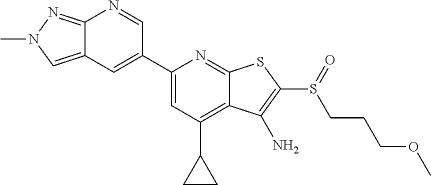 | *** |
| 172 | 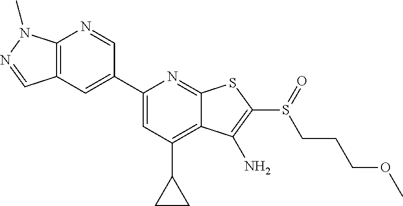 [SMILES: COCCCS(=O)c1sc2nc(-c3cnc4c(cnn4C)c3)cc(C3CC3)c2c1N] | ** |
| 173 | 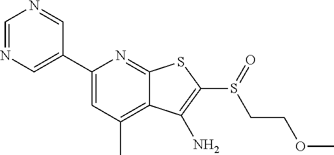 [SMILES: COCCS(=O)c1sc2nc(-c3cncnc3)cc(C)c2c1N] | *** |
| 174 | 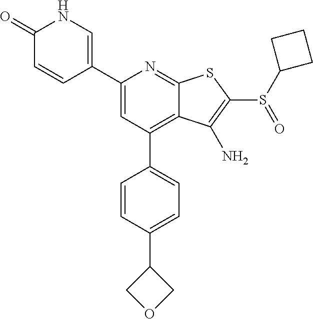 [SMILES: Nc1c(S(=O)C2CCC2)sc2nc(-c3ccc(=O)[nH]c3)cc(-c3ccc(C4COC4)cc3)c12] | ** |
| 175 | 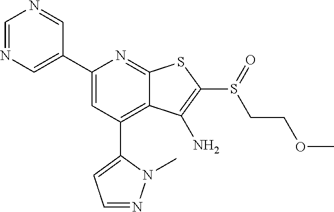 [SMILES: COCCS(=O)c1sc2nc(-c3cncnc3)cc(-c3ccnn3C)c2c1N] | ** |
| 176 | 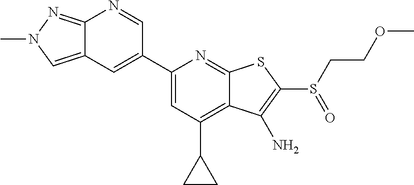 | *** |
| 177 | 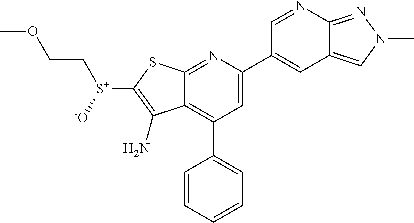 | ** |
| 178 | 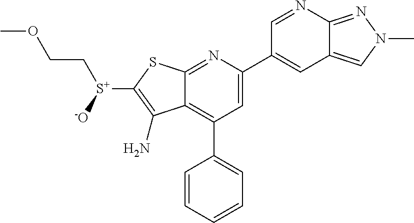 | * |
| 179 | 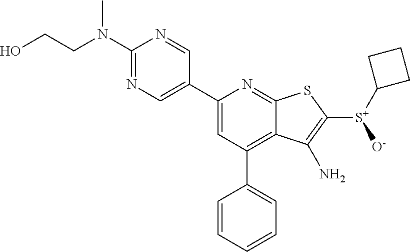 [SMILES: CN(CCO)c1ncc(-c2cc(-c3ccccc3)c3c(N)c([S@+]([O-])C4CCC4)sc3n2)cn1] | *** |
| 180 | 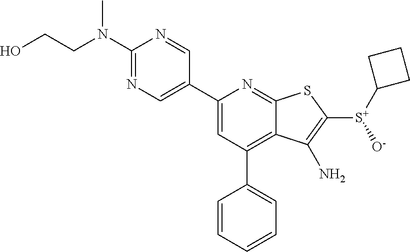 [SMILES: CN(CCO)c1ncc(-c2cc(-c3ccccc3)c3c(N)c([S@@+]([O-])C4CCC4)sc3n2)cn1] | * |
| 181 | 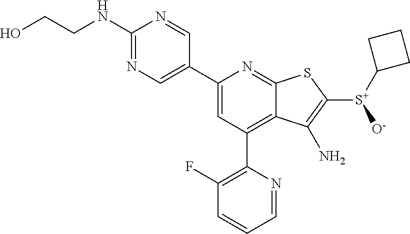 [SMILES: Nc1c([S@+]([O-])C2CCC2)sc2nc(-c3cnc(NCCO)nc3)cc(-c3ncccc3F)c12] | *** |
| 182 | 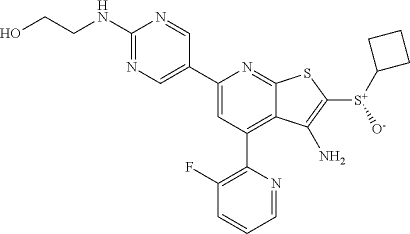 [SMILES: Nc1c([S@@+]([O-])C2CCC2)sc2nc(-c3cnc(NCCO)nc3)cc(-c3ncccc3F)c12] | * |
| 183 | 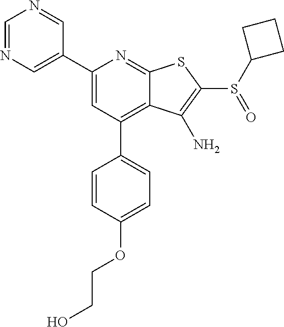 [SMILES: Nc1c(S(=O)C2CCC2)sc2nc(-c3cncnc3)cc(-c3ccc(OCCO)cc3)c12] | *** |
| 184 | 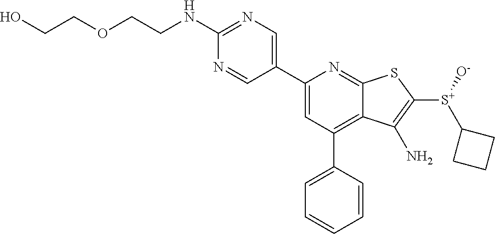 [SMILES: Nc1c([S@+]([O-])C2CCC2)sc2nc(-c3cnc(NCCOCCO)nc3)cc(-c3ccccc3)c12] | *** |
| 185 | 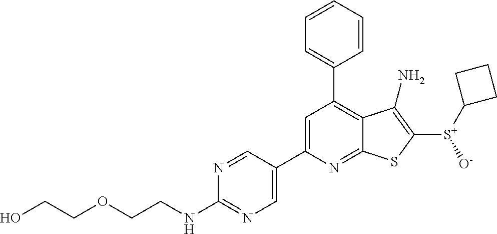 [SMILES: Nc1c([S@@+]([O-])C2CCC2)sc2nc(-c3cnc(NCCOCCO)nc3)cc(-c3ccccc3)c12] | * |
| 186 | 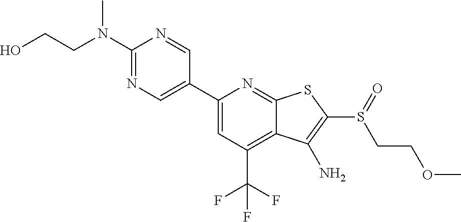 [SMILES: COCCS(=O)c1sc2nc(-c3cnc(N(C)CCO)nc3)cc(C(F)(F)F)c2c1N] | ** |
| 187 | 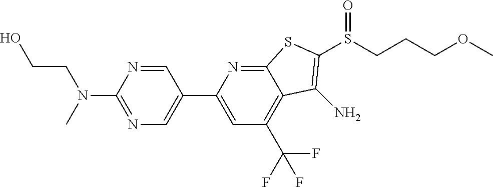 [SMILES: COCCCS(=O)c1sc2nc(-c3cnc(N(C)CCO)nc3)cc(C(F)(F)F)c2c1N] | ** |
| 188 | 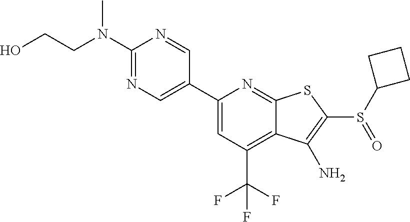 [SMILES: CN(CCO)c1ncc(-c2cc(C(F)(F)F)c3c(N)c(S(=O)C4CCC4)sc3n2)cn1] | ** |
| 189 | 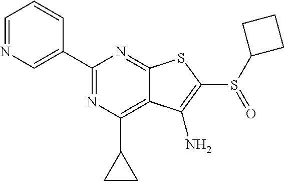 [SMILES: Nc1c(S(=O)C2CCC2)sc2nc(-c3cccnc3)nc(C3CC3)c12] | *** |
| 190 | 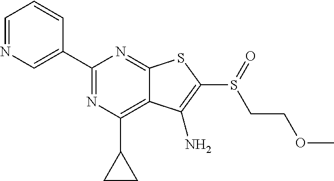 [SMILES: COCCS(=O)c1sc2nc(-c3cccnc3)nc(C3CC3)c2c1N] | *** |
| 191 | 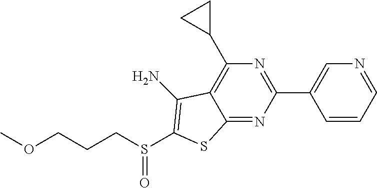 [SMILES: COCCCS(=O)c1sc2nc(-c3cccnc3)nc(C3CC3)c2c1N] | ** |
| 192 | 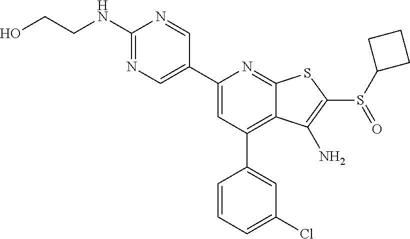 [SMILES: Nc1c(S(=O)C2CCC2)sc2nc(-c3cnc(NCCO)nc3)cc(-c3cccc(Cl)c3)c12] | *** |
| 193 | 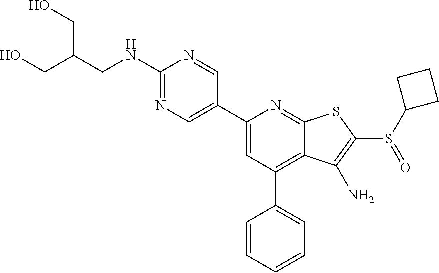 [SMILES: Nc1c(S(=O)C2CCC2)sc2nc(-c3cnc(NCC(CO)CO)nc3)cc(-c3ccccc3)c12] | *** |
| 194 | 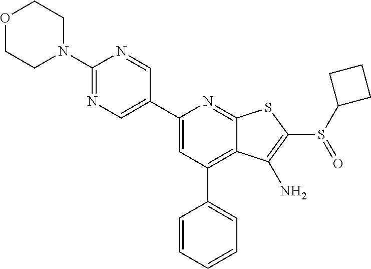 [SMILES: Nc1c(S(=O)C2CCC2)sc2nc(-c3cnc(N4CCOCC4)nc3)cc(-c3ccccc3)c12] | ** |
| 195 | 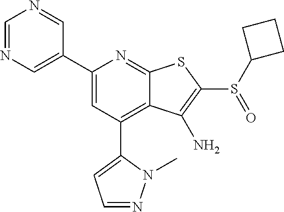 [SMILES: Cn1nccc1-c1cc(-c2cncnc2)nc2sc(S(=O)C3CCC3)c(N)c12] | *** |
| 196 | 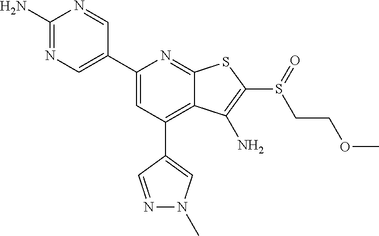 [SMILES: COCCS(=O)c1sc2nc(-c3cnc(N)nc3)cc(-c3cnn(C)c3)c2c1N] | *** |
| 197 | 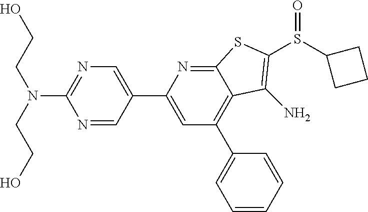 [SMILES: Nc1c(S(=O)C2CCC2)sc2nc(-c3cnc(N(CCO)CCO)nc3)cc(-c3ccccc3)c12] | *** |
| 198 | 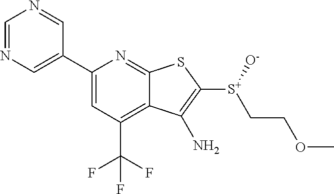 [SMILES: COCC[S@@+]([O-])c1sc2nc(-c3cncnc3)cc(C(F)(F)F)c2c1N] | *** |
| 199 | 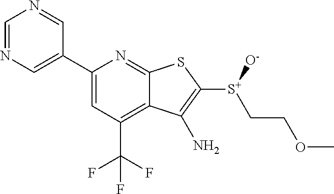 [SMILES: COCC[S@+]([O-])c1sc2nc(-c3cncnc3)cc(C(F)(F)F)c2c1N] | * |
| 200 | 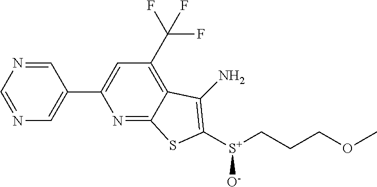 [SMILES: COCCC[S@@+]([O-])c1sc2nc(-c3cncnc3)cc(C(F)(F)F)c2c1N] | *** |
| 201 | 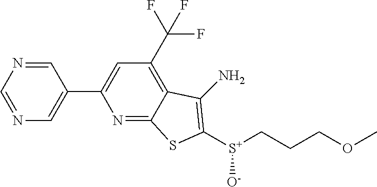 [SMILES: COCCC[S@+]([O-])c1sc2nc(-c3cncnc3)cc(C(F)(F)F)c2c1N] | * |
| 202 | 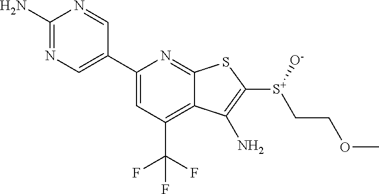 [SMILES: COCC[S@@+]([O-])c1sc2nc(-c3cnc(N)nc3)cc(C(F)(F)F)c2c1N] | *** |
| 203 | 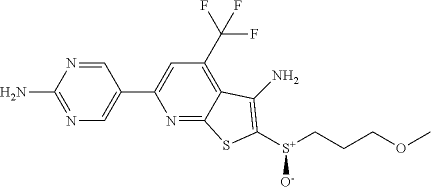 [SMILES: COCCC[S@@+]([O-])c1sc2nc(-c3cnc(N)nc3)cc(C(F)(F)F)c2c1N] | *** |
| 204 | 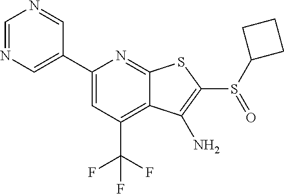 [SMILES: Nc1c(S(=O)C2CCC2)sc2nc(-c3cncnc3)cc(C(F)(F)F)c12] | *** |
| 205 | 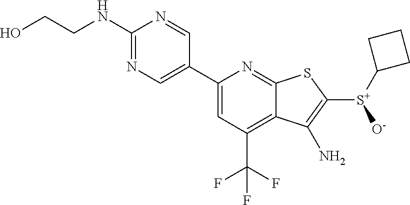 [SMILES: Nc1c([S@+]([O-])C2CCC2)sc2nc(-c3cnc(NCCO)nc3)cc(C(F)(F)F)c12] | *** |
| 206 | 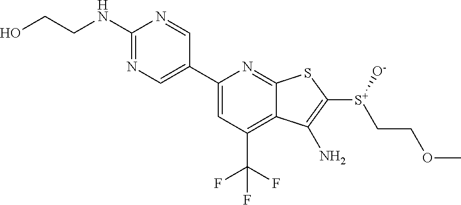 [SMILES: COCC[S@@+]([O-])c1sc2nc(-c3cnc(NCCO)nc3)cc(C(F)(F)F)c2c1N] | *** |
| 207 | 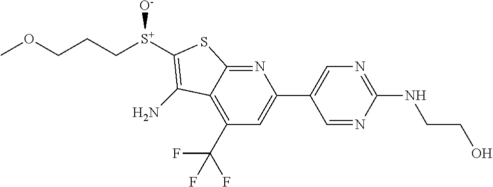 [SMILES: COCCC[S@@+]([O-])c1sc2nc(-c3cnc(NCCO)nc3)cc(C(F)(F)F)c2c1N] | *** |
| 208 | 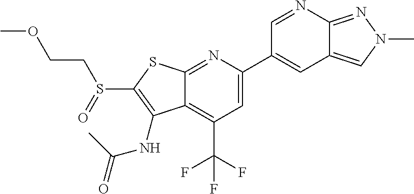 | * |
| 209 | 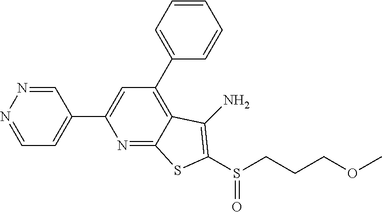 [SMILES: COCCCS(=O)c1sc2nc(-c3ccnnc3)cc(-c3ccccc3)c2c1N] | *** |
| 210 | 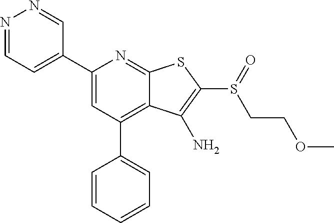 [SMILES: COCCS(=O)c1sc2nc(-c3ccnnc3)cc(-c3ccccc3)c2c1N] | *** |
| 211 | 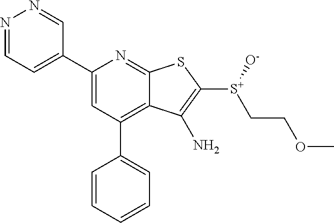 [SMILES: COCC[S@@+]([O-])c1sc2nc(-c3ccnnc3)cc(-c3ccccc3)c2c1N] | *** |
| 212 | 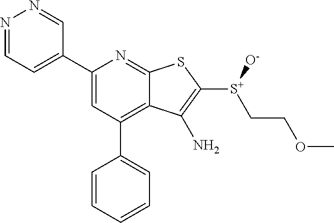 [SMILES: COCC[S@+]([O-])c1sc2nc(-c3ccnnc3)cc(-c3ccccc3)c2c1N] | * |
| 213 | 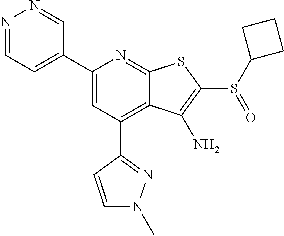 [SMILES: Cn1ccc(-c2cc(-c3ccnnc3)nc3sc(S(=O)C4CCC4)c(N)c23)n1] | *** |
| 214 | 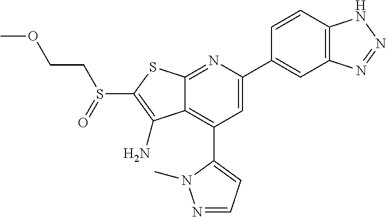 [SMILES: COCCS(=O)c1sc2nc(-c3ccc4[nH]nnc4c3)cc(-c3ccnn3C)c2c1N] | *** |
| 215 | 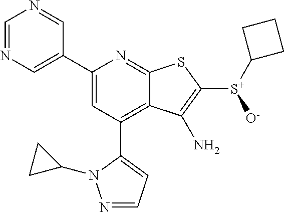 [SMILES: Nc1c([S@+]([O-])C2CCC2)sc2nc(-c3cncnc3)cc(-c3ccnn3C3CC3)c12] | *** |
| 216 | 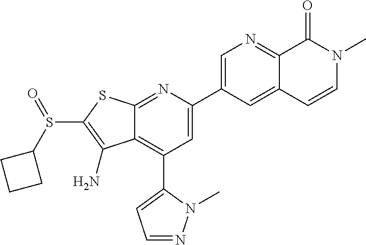 [SMILES: Cn1nccc1-c1cc(-c2cnc3c(=O)n(C)ccc3c2)nc2sc(S(=O)C3CCC3)c(N)c12] | *** |
| 217 | 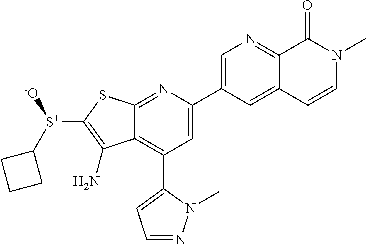 | *** |
| 218 | 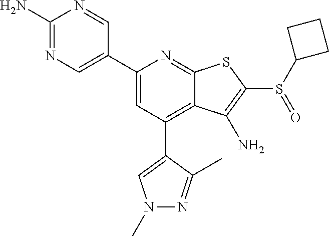 [SMILES: Cc1nn(C)cc1-c1cc(-c2cnc(N)nc2)nc2sc(S(=O)C3CCC3)c(N)c12] | *** |
| 219 | 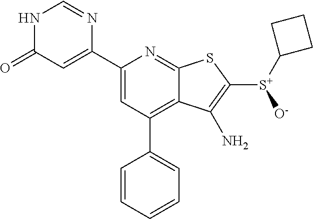 [SMILES: Nc1c([S@+]([O-])C2CCC2)sc2nc(-c3cc(=O)[nH]cn3)cc(-c3ccccc3)c12] | *** |
| 220 | 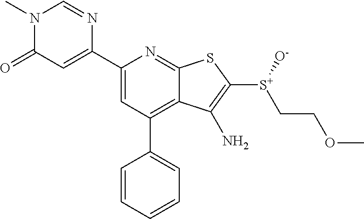 [SMILES: COCC[S@@+]([O-])c1sc2nc(-c3cc(=O)n(C)cn3)cc(-c3ccccc3)c2c1N] | *** |
| 221 | 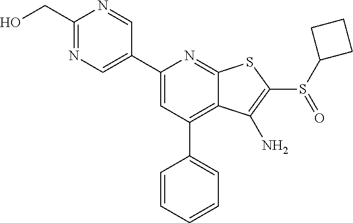 [SMILES: Nc1c(S(=O)C2CCC2)sc2nc(-c3cnc(CO)nc3)cc(-c3ccccc3)c12] | *** |
| 222 | 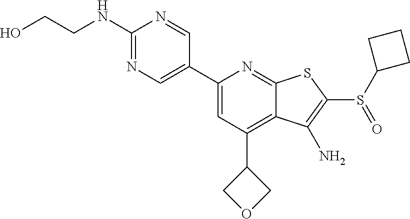 [SMILES: Nc1c(S(=O)C2CCC2)sc2nc(-c3cnc(NCCO)nc3)cc(C3COC3)c12] | *** |
| 223 | 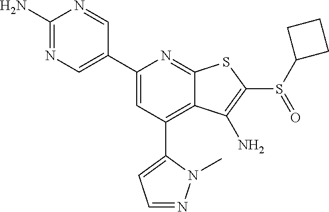 [SMILES: Cn1nccc1-c1cc(-c2cnc(N)nc2)nc2sc(S(=O)C3CCC3)c(N)c12] | *** |
| 224 | 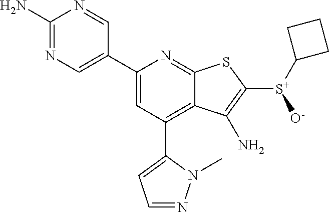 [SMILES: Cn1nccc1-c1cc(-c2cnc(N)nc2)nc2sc([S@+]([O-])C3CCC3)c(N)c12] | *** |
| 225 | 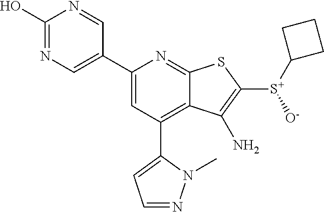 [SMILES: Cn1nccc1-c1cc(-c2cnc(O)nc2)nc2sc([S@@+]([O-])C3CCC3)c(N)c12] | * |
| 226 | 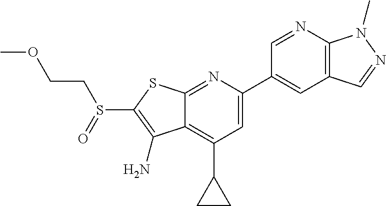 [SMILES: COCCS(=O)c1sc2nc(-c3cnc4c(cnn4C)c3)cc(C3CC3)c2c1N] | *** |
| 227 | 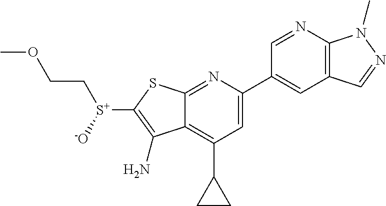 | *** |
| 228 | 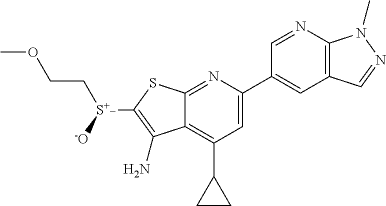 | * |
| 229 | 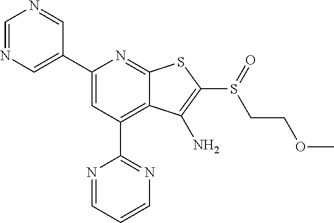 [SMILES: COCCS(=O)c1sc2nc(-c3cncnc3)cc(-c3ncccn3)c2c1N] | *** |
| 230 | 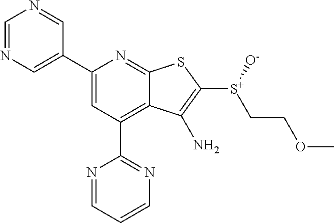 [SMILES: COCC[S@@+]([O-])c1sc2nc(-c3cncnc3)cc(-c3ncccn3)c2c1N] | *** |
| 231 | 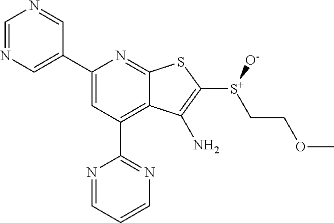 [SMILES: COCC[S@+]([O-])c1sc2nc(-c3cncnc3)cc(-c3ncccn3)c2c1N] | * |
| 232 | 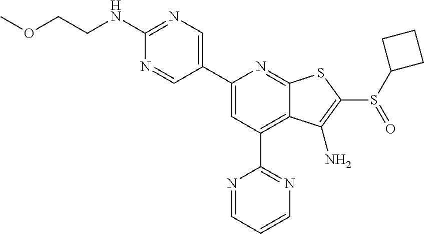 [SMILES: COCCNc1ncc(-c2cc(-c3ncccn3)c3c(N)c(S(=O)C4CCC4)sc3n2)cn1] | *** |
| 233 | 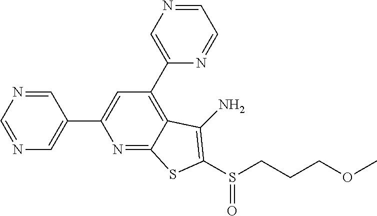 [SMILES: COCCCS(=O)c1sc2nc(-c3cncnc3)cc(-c3cnccn3)c2c1N] | *** |
| 234 | 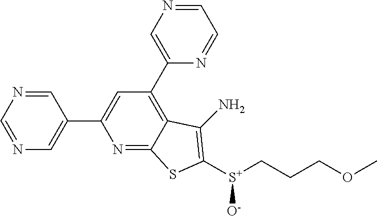 [SMILES: COCCC[S@@+]([O-])c1sc2nc(-c3cncnc3)cc(-c3cnccn3)c2c1N] | * |
| 235 | 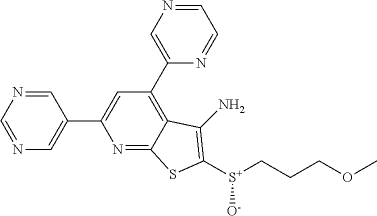 [SMILES: COCCC[S@+]([O-])c1sc2nc(-c3cncnc3)cc(-c3cnccn3)c2c1N] | *** |
| 236 | 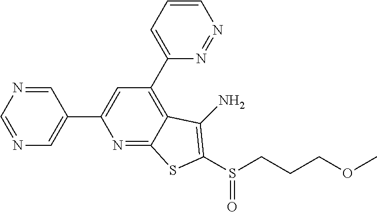 [SMILES: COCCCS(=O)c1sc2nc(-c3cncnc3)cc(-c3cccnn3)c2c1N] | *** |
| 237 | 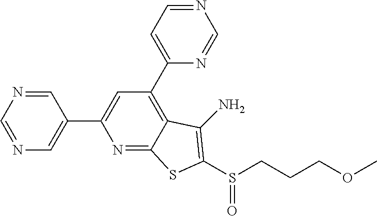 [SMILES: COCCCS(=O)c1sc2nc(-c3cncnc3)cc(-c3ccncn3)c2c1N] | *** |
| 238 | 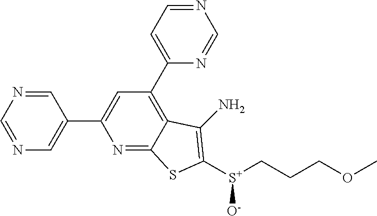 [SMILES: COCCC[S@@+]([O-])c1sc2nc(-c3cncnc3)cc(-c3ccncn3)c2c1N] | *** |
| 239 | 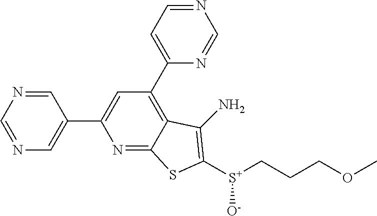 [SMILES: COCCC[S@+]([O-])c1sc2nc(-c3cncnc3)cc(-c3ccncn3)c2c1N] | * |
| 240 | 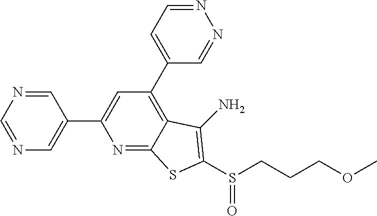 [SMILES: COCCCS(=O)c1sc2nc(-c3cncnc3)cc(-c3ccnnc3)c2c1N] | ** |
| 241 | 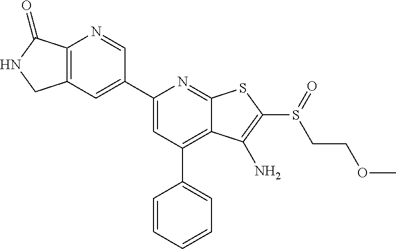 [SMILES: COCCS(=O)c1sc2nc(-c3cnc4c(c3)CNC4=O)cc(-c3ccccc3)c2c1N] | *** |
| 242 | 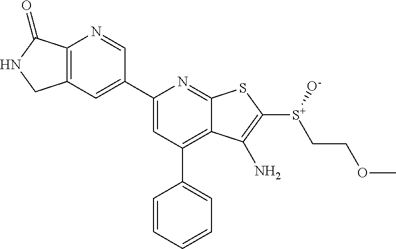 [SMILES: COCC[S@@+]([O-])c1sc2nc(-c3cnc4c(c3)CNC4=O)cc(-c3ccccc3)c2c1N] | *** |
| 243 | 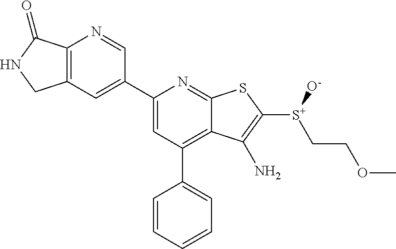 [SMILES: COCC[S@+]([O-])c1sc2nc(-c3cnc4c(c3)CNC4=O)cc(-c3ccccc3)c2c1N] | * |
| 244 | 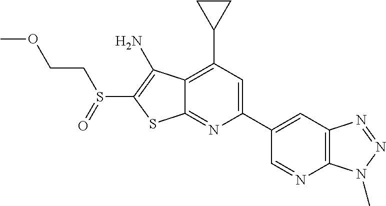 [SMILES: COCCS(=O)c1sc2nc(-c3cnc4c(c3)nnn4C)cc(C3CC3)c2c1N] | *** |
| 245 | 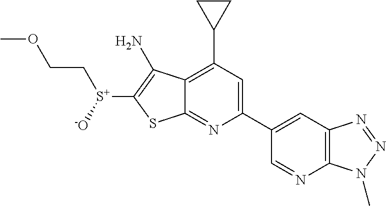 | *** |
| 246 | 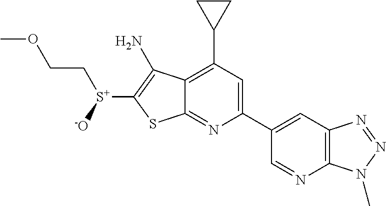 | * |
| 247 | 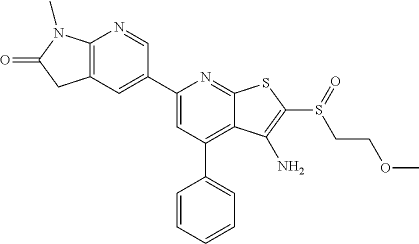 [SMILES: COCCS(=O)c1sc2nc(-c3cnc4c(c3)CC(=O)N4C)cc(-c3ccccc3)c2c1N] | *** |
| 248 | 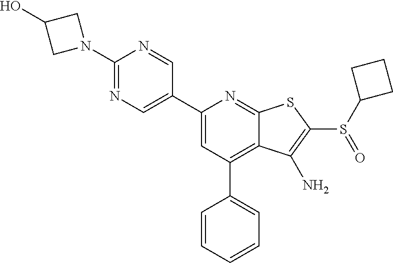 [SMILES: Nc1c(S(=O)C2CCC2)sc2nc(-c3cnc(N4CC(O)C4)nc3)cc(-c3ccccc3)c12] | *** |
| 249 | 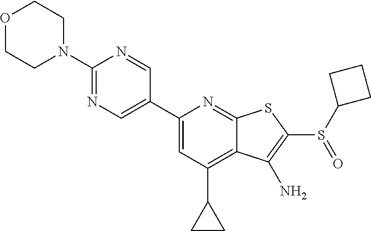 [SMILES: Nc1c(S(=O)C2CCC2)sc2nc(-c3cnc(N4CCOCC4)nc3)cc(C3CC3)c12] | *** |
| 250 | 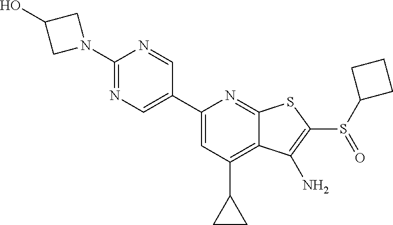 [SMILES: Nc1c(S(=O)C2CCC2)sc2nc(-c3cnc(N4CC(O)C4)nc3)cc(C3CC3)c12] | *** |
| 251 | 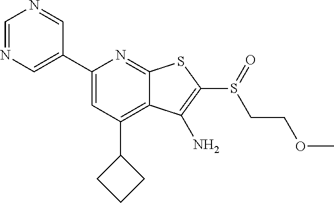 [SMILES: COCCS(=O)c1sc2nc(-c3cncnc3)cc(C3CCC3)c2c1N] | *** |
| 252 | 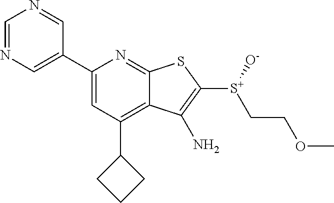 [SMILES: COCC[S@@+]([O-])c1sc2nc(-c3cncnc3)cc(C3CCC3)c2c1N] | *** |
| 253 | 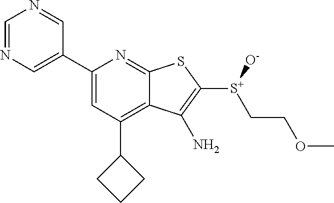 [SMILES: COCC[S@+]([O-])c1sc2nc(-c3cncnc3)cc(C3CCC3)c2c1N] | * |
| 254 | 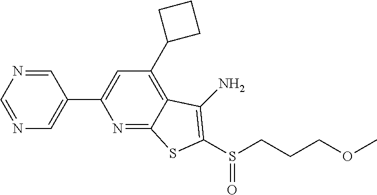 [SMILES: COCCCS(=O)c1sc2nc(-c3cncnc3)cc(C3CCC3)c2c1N] | *** |
| 255 | 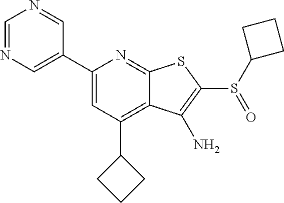 [SMILES: Nc1c(S(=O)C2CCC2)sc2nc(-c3cncnc3)cc(C3CCC3)c12] | *** |
| 256 | 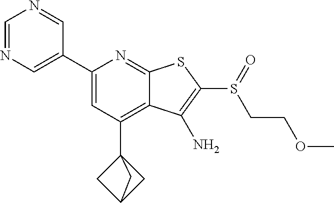 [SMILES: COCCS(=O)c1sc2nc(-c3cncnc3)cc(C34CC(C3)C4)c2c1N] | *** |
| 257 | 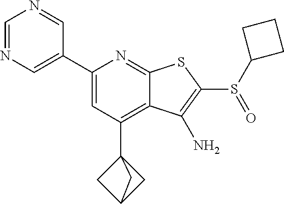 [SMILES: Nc1c(S(=O)C2CCC2)sc2nc(-c3cncnc3)cc(C34CC(C3)C4)c12] | *** |
| 258 | 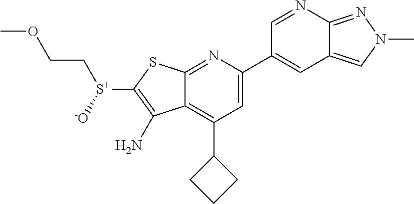 | *** |
| 259 | 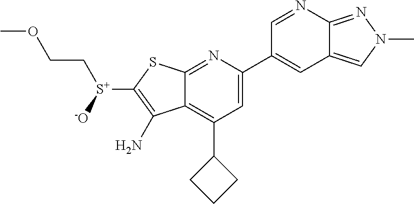 | * |
| 260 | 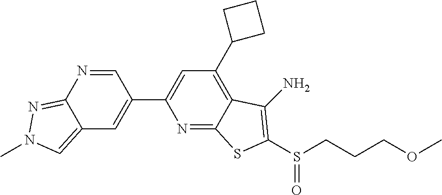 | *** |
| 261 | 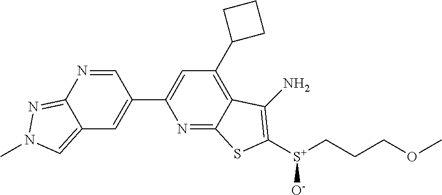 | *** |
| 262 | 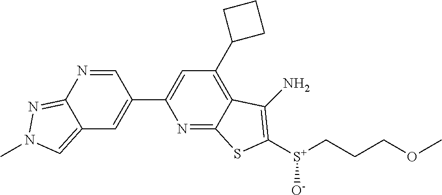 | * |
| 263 | 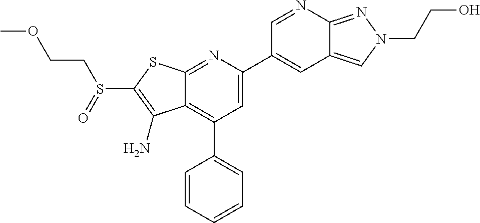 [SMILES: COCCS(=O)c1sc2nc(-c3cnc4nn(CCO)cc4c3)cc(-c3ccccc3)c2c1N] | *** |
| 264 | 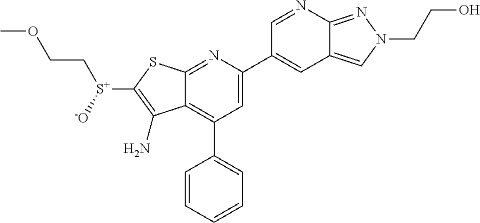 | *** |
| 265 | 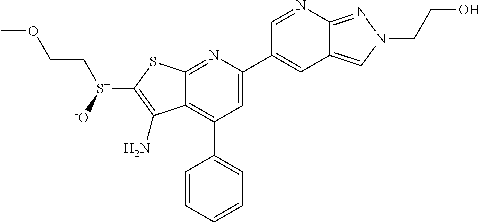 | * |
| 266 | 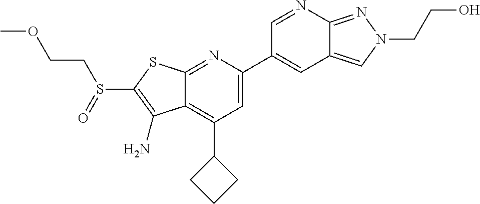 [SMILES: COCCS(=O)c1sc2nc(-c3cnc4nn(CCO)cc4c3)cc(C3CCC3)c2c1N] | *** |
| 267 | 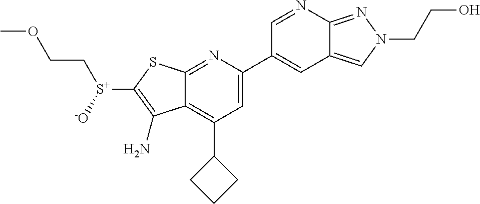 | *** |
| 268 | 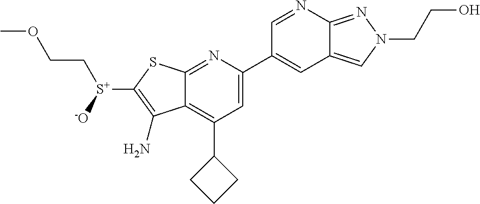 | * |
| 269 | 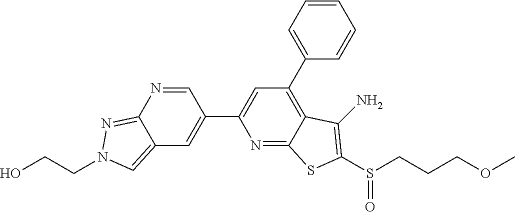 [SMILES: COCCCS(=O)c1sc2nc(-c3cnc4nn(CCO)cc4c3)cc(-c3ccccc3)c2c1N] | *** |
| 270 | 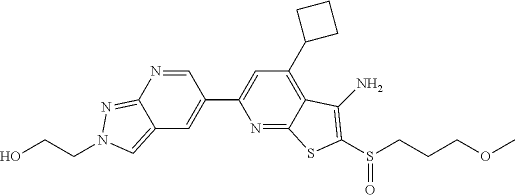 [SMILES: COCCCS(=O)c1sc2nc(-c3cnc4nn(CCO)cc4c3)cc(C3CCC3)c2c1N] | *** |
| 271 | 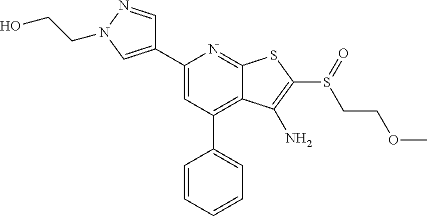 [SMILES: COCCS(=O)c1sc2nc(-c3cnn(CCO)c3)cc(-c3ccccc3)c2c1N] | *** |
| 272 | 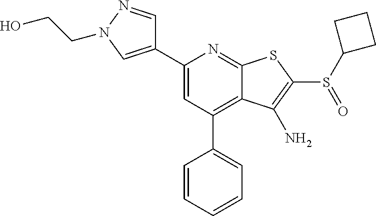 [SMILES: Nc1c(S(=O)C2CCC2)sc2nc(-c3cnn(CCO)c3)cc(-c3ccccc3)c12] | *** |
| 273 | 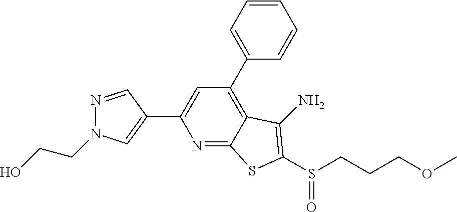 [SMILES: COCCCS(=O)c1sc2nc(-c3cnn(CCO)c3)cc(-c3ccccc3)c2c1N] | *** |
| 274 | 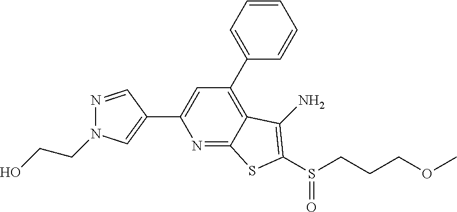 [SMILES: COCCCS(=O)c1sc2nc(-c3cnn(CCO)c3)cc(-c3ccccc3)c2c1N] | *** |
| 275 | 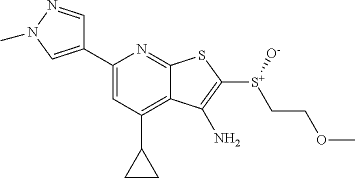 | *** |
| 276 | 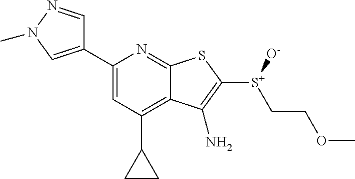 | * |
| 277 | 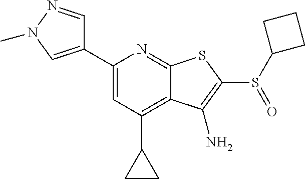 [SMILES: Cn1cc(-c2cc(C3CC3)c3c(N)c(S(=O)C4CCC4)sc3n2)cn1] | *** |
| 278 | 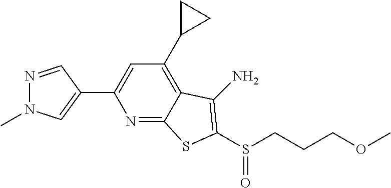 | *** |
| 279 | 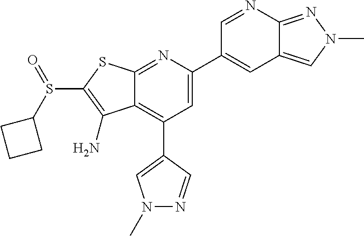 [SMILES: Cn1cc(-c2cc(-c3cnc4nn(C)cc4c3)nc3sc(S(=O)C4CCC4)c(N)c23)cn1] | ** |
| 280 | 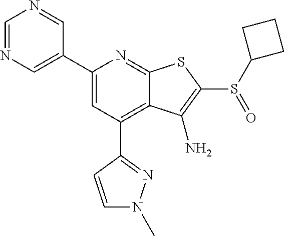 [SMILES: Cn1ccc(-c2cc(-c3cncnc3)nc3sc(S(=O)C4CCC4)c(N)c23)n1] | *** |
| 281 | 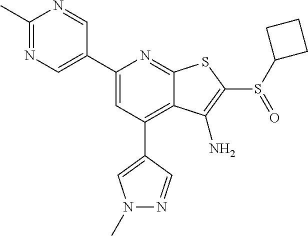 [SMILES: Cc1ncc(-c2cc(-c3cnn(C)c3)c3c(N)c(S(=O)C4CCC4)sc3n2)cn1] | *** |
| 282 | 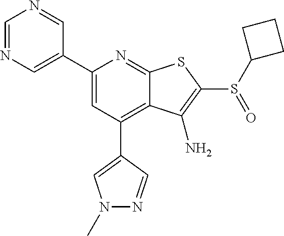 [SMILES: Cn1cc(-c2cc(-c3cncnc3)nc3sc(S(=O)C4CCC4)c(N)c23)cn1] | *** |
| 283 | 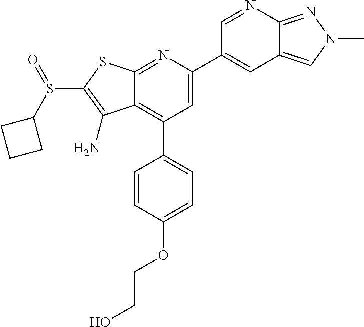 [SMILES: Cn1cc2cc(-c3cc(-c4ccc(OCCO)cc4)c4c(N)c(S(=O)C5CCC5)sc4n3)cnc2n1] | *** |
| 284 | 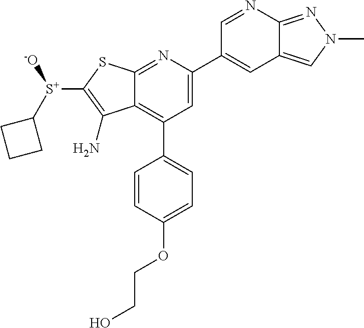 | *** |
| 285 | 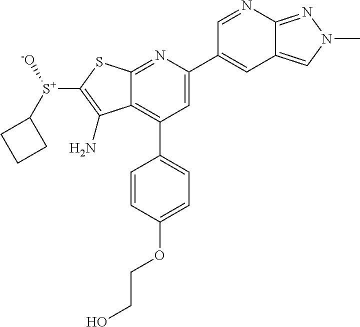 | ** |
| 286 | 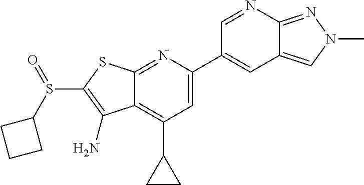 [SMILES: Cn1cc2cc(-c3cc(C4CC4)c4c(N)c(S(=O)C5CCC5)sc4n3)cnc2n1] | *** |
| 287 | 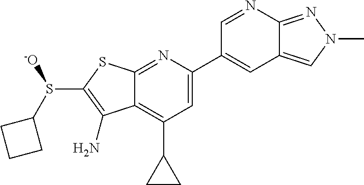 | *** |
| 288 | 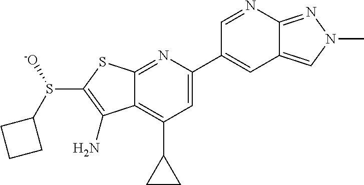 | * |
| 289 | 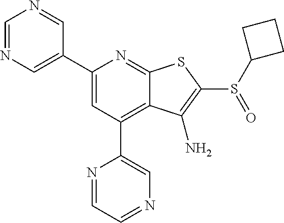 [SMILES: Nc1c(S(=O)C2CCC2)sc2nc(-c3cncnc3)cc(-c3cnccn3)c12] | *** |
| 290 | 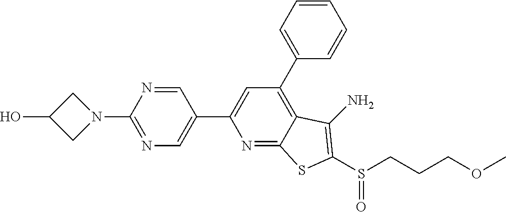 [SMILES: COCCCS(=O)c1sc2nc(-c3cnc(N4CC(O)C4)nc3)cc(-c3ccccc3)c2c1N] | ** |
| 300 | 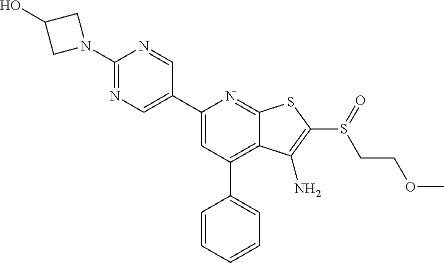 [SMILES: COCCS(=O)c1sc2nc(-c3cnc(N4CC(O)C4)nc3)cc(-c3ccccc3)c2c1N] | ** |
| 301 | 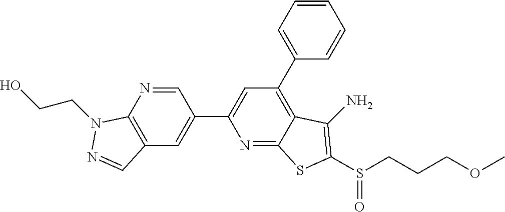 [SMILES: COCCCS(=O)c1sc2nc(-c3cnc4c(cnn4CCO)c3)cc(-c3ccccc3)c2c1N] | ** |
| 302 | 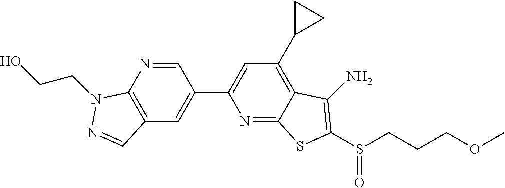 [SMILES: COCCCS(=O)c1sc2nc(-c3cnc4c(cnn4CCO)c3)cc(C3CC3)c2c1N] | ** |
| 303 | 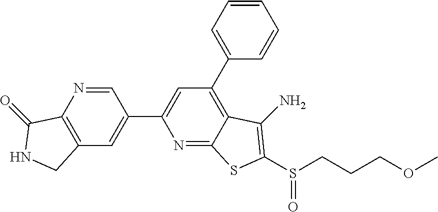 [SMILES: COCCCS(=O)c1sc2nc(-c3cnc4c(c3)CNC4=O)cc(-c3ccccc3)c2c1N] | *** |
| 304 | 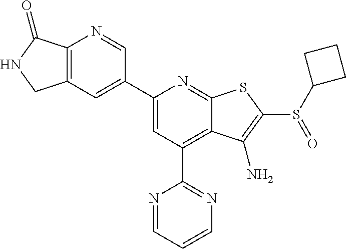 [SMILES: Nc1c(S(=O)C2CCC2)sc2nc(-c3cnc4c(c3)CNC4=O)cc(-c3ncccn3)c12] | ** |
| 305 | 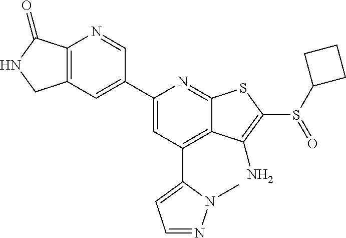 [SMILES: Cn1nccc1-c1cc(-c2cnc3c(c2)CNC3=O)nc2sc(S(=O)C3CCC3)c(N)c12] | *** |
| 306 | 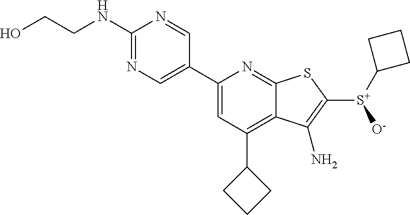 [SMILES: Nc1c([S@+]([O-])C2CCC2)sc2nc(-c3cnc(NCCO)nc3)cc(C3CCC3)c12] | *** |
| 307 | 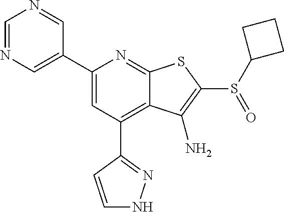 [SMILES: Nc1c(S(=O)C2CCC2)sc2nc(-c3cncnc3)cc(-c3cc[nH]n3)c12] | *** |
| 308 | 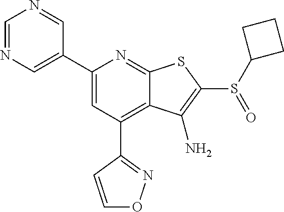 [SMILES: Nc1c(S(=O)C2CCC2)sc2nc(-c3cncnc3)cc(-c3ccon3)c12] | *** |
| 309 | 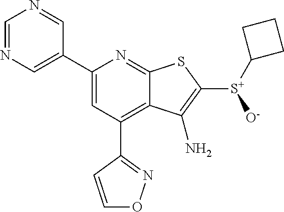 [SMILES: Nc1c([S@+]([O-])C2CCC2)sc2nc(-c3cncnc3)cc(-c3ccon3)c12] | *** |
| 310 | 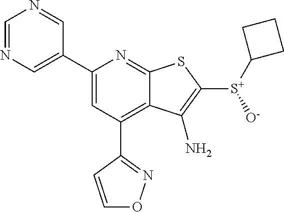 [SMILES: Nc1c([S@@+]([O-])C2CCC2)sc2nc(-c3cncnc3)cc(-c3ccon3)c12] | * |
| 311 | 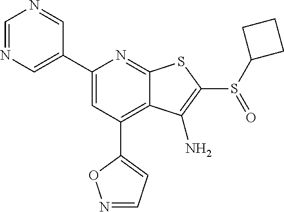 [SMILES: Nc1c(S(=O)C2CCC2)sc2nc(-c3cncnc3)cc(-c3ccno3)c12] | *** |
| 312 | 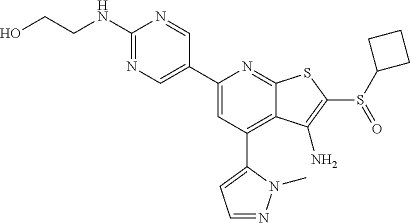 [SMILES: Cn1nccc1-c1cc(-c2cnc(NCCO)nc2)nc2sc(S(=O)C3CCC3)c(N)c12] | *** |
| 313 | 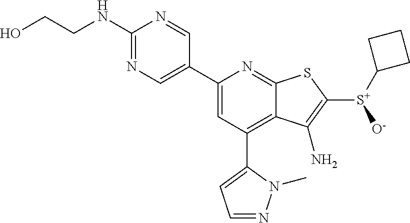 [SMILES: Cn1nccc1-c1cc(-c2cnc(NCCO)nc2)nc2sc([S@+]([O-])C3CCC3)c(N)c12] | *** |
| 314 | 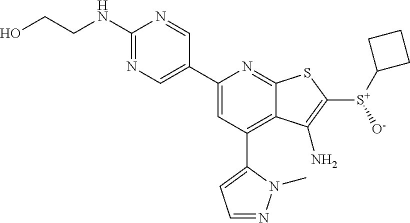 [SMILES: Cn1nccc1-c1cc(-c2cnc(NCCO)nc2)nc2sc([S@@+]([O-])C3CCC3)c(N)c12] | * |
| 315 | 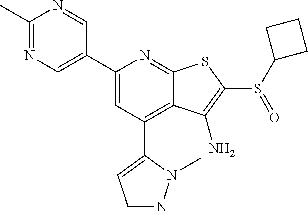 [SMILES: Cc1ncc(-c2cc(C3=CCNN3C)c3c(N)c(S(=O)C4CCC4)sc3n2)cn1] | ** |
| 316 | 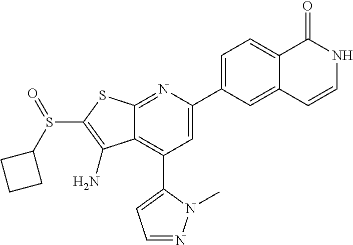 [SMILES: Cn1nccc1-c1cc(-c2ccc3c(=O)[nH]ccc3c2)nc2sc(S(=O)C3CCC3)c(N)c12] | ** |
| 317 | 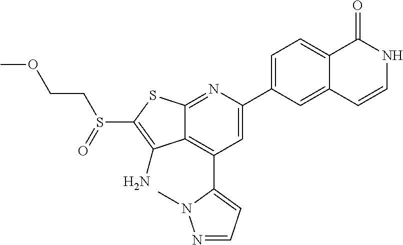 [SMILES: COCCS(=O)c1sc2nc(-c3ccc4c(=O)[nH]ccc4c3)cc(-c3ccnn3C)c2c1N] | *** |
| 318 | 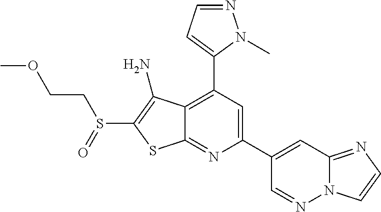 [SMILES: COCCS(=O)c1sc2nc(-c3cnn4ccnc4c3)cc(-c3ccnn3C)c2c1N] | *** |
| 319 | 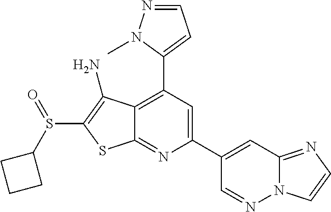 [SMILES: Cn1nccc1-c1cc(-c2cnn3ccnc3c2)nc2sc(S(=O)C3CCC3)c(N)c12] | *** |
| 320 | 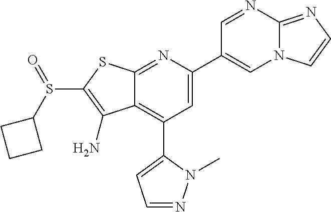 [SMILES: Cn1nccc1-c1cc(-c2cnc3nccn3c2)nc2sc(S(=O)C3CCC3)c(N)c12] | *** |
| 321 | 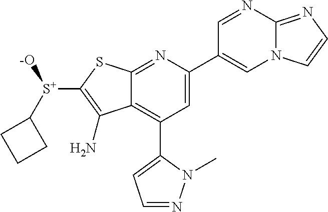 | *** |
| 322 | 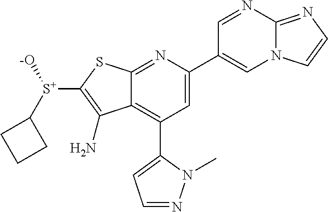 | * |
| 323 | 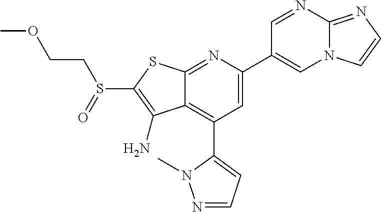 [SMILES: COCCS(=O)c1sc2nc(-c3cnc4nccn4c3)cc(-c3ccnn3C)c2c1N] | *** |
| 324 | 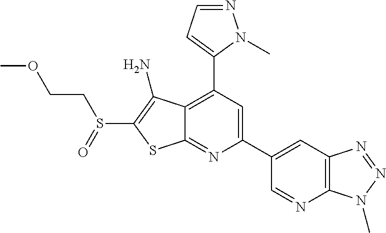 [SMILES: COCCS(=O)c1sc2nc(-c3cnc4c(c3)nnn4C)cc(-c3ccnn3C)c2c1N] | *** |
| 325 | 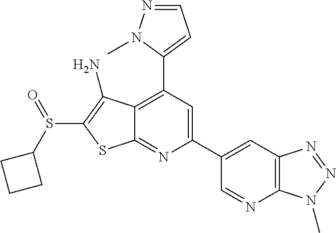 [SMILES: Cn1nccc1-c1cc(-c2cnc3c(c2)nnn3C)nc2sc(S(=O)C3CCC3)c(N)c12] | *** |
| 326 | 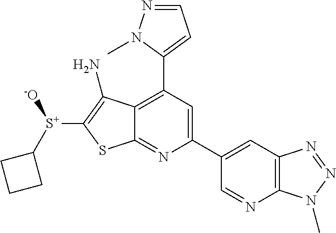 | *** |
| 327 | 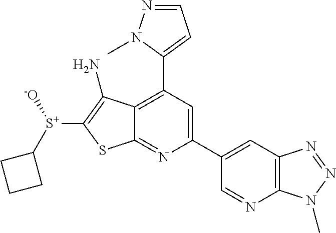 | * |
| 328 | 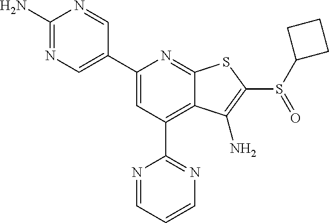 [SMILES: Nc1ncc(-c2cc(-c3ncccn3)c3c(N)c(S(=O)C4CCC4)sc3n2)cn1] | *** |
| 329 | 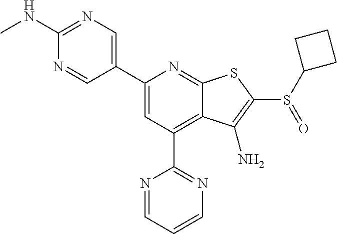 [SMILES: CNc1ncc(-c2cc(-c3ncccn3)c3c(N)c(S(=O)C4CCC4)sc3n2)cn1] | *** |
| 330 | 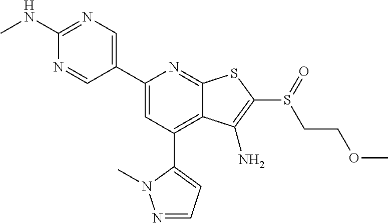 | *** |
| 331 | 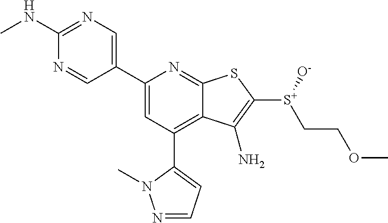 | *** |
| 332 | 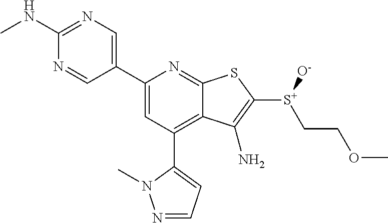 | * |
| 333 | 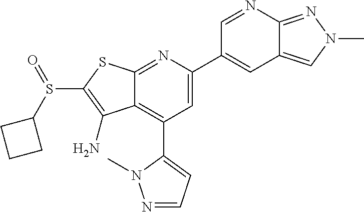 [SMILES: Cn1cc2cc(-c3cc(-c4ccnn4C)c4c(N)c(S(=O)C5CCC5)sc4n3)cnc2n1] | *** |
| 334 | 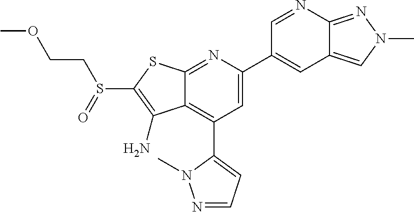 | *** |
| 335 | 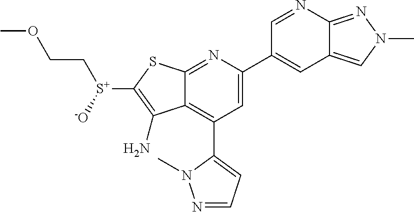 | *** |
| 336 | 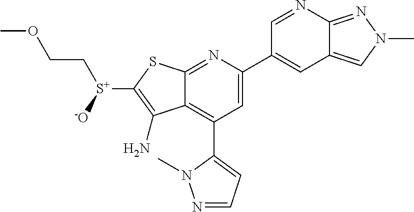 | * |
| 337 | 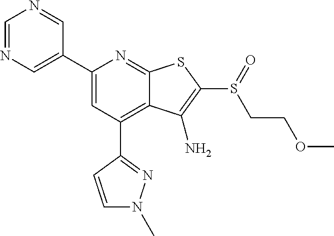 [SMILES: COCCS(=O)c1sc2nc(-c3cncnc3)cc(-c3ccn(C)n3)c2c1N] | *** |
| 338 | 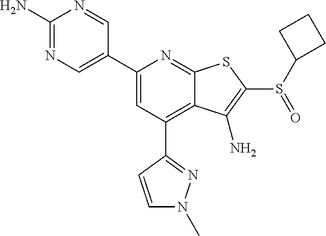 [SMILES: Cn1ccc(-c2cc(-c3cnc(N)nc3)nc3sc(S(=O)C4CCC4)c(N)c23)n1] | *** |
| 339 | 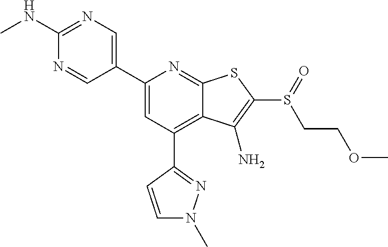 | *** |
| 340 | 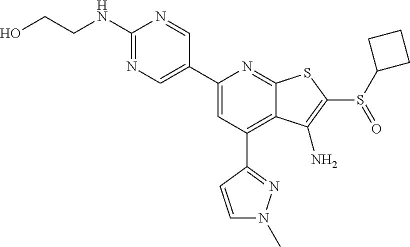 [SMILES: Cn1ccc(-c2cc(-c3cnc(NCCO)nc3)nc3sc(S(=O)C4CCC4)c(N)c23)n1] | *** |
| 341 | 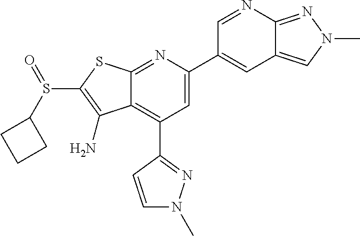 [SMILES: Cn1ccc(-c2cc(-c3cnc4nn(C)cc4c3)nc3sc(S(=O)C4CCC4)c(N)c23)n1] | *** |
| 342 | 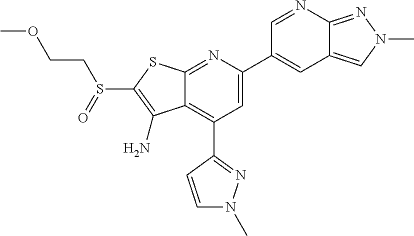 | *** |
| 343 | 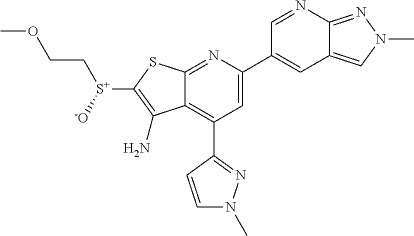 | *** |
| 344 | 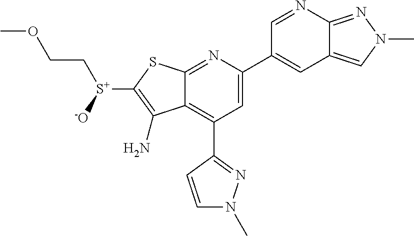 | * |
| 345 | 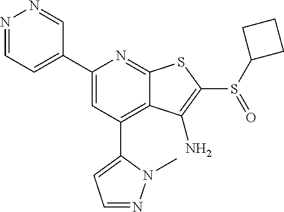 [SMILES: Cn1nccc1-c1cc(-c2ccnnc2)nc2sc(S(=O)C3CCC3)c(N)c12] | *** |
| 346 | 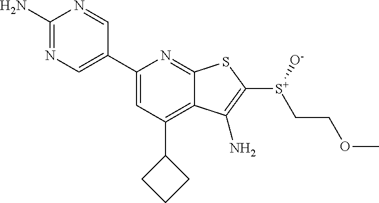 [SMILES: COCC[S@@+]([O-])c1sc2nc(-c3cnc(N)nc3)cc(C3CCC3)c2c1N] | *** |
| 347 | 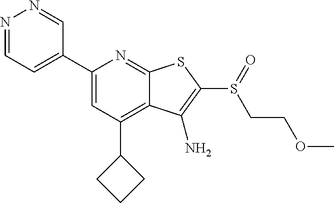 [SMILES: COCCS(=O)c1sc2nc(-c3ccnnc3)cc(C3CCC3)c2c1N] | *** |
| 348 | 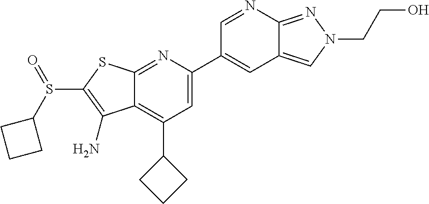 [SMILES: Nc1c(S(=O)C2CCC2)sc2nc(-c3cnc4nn(CCO)cc4c3)cc(C3CCC3)c12] | ** |
| 349 | 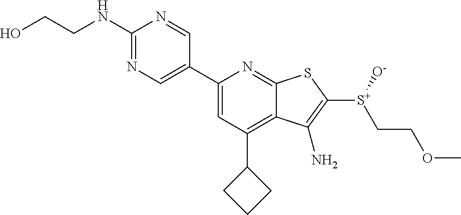 [SMILES: COCC[S@@+]([O-])c1sc2nc(-c3cnc(NCCO)nc3)cc(C3CCC3)c2c1N] | *** |
| 350 | 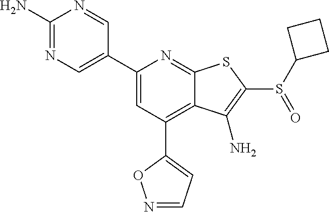 [SMILES: Nc1ncc(-c2cc(-c3ccno3)c3c(N)c(S(=O)C4CCC4)sc3n2)cn1] | *** |
| 351 | 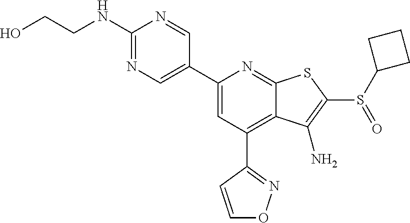 [SMILES: Nc1c(S(=O)C2CCC2)sc2nc(-c3cnc(NCCO)nc3)cc(-c3ccon3)c12] | *** |
| 352 | 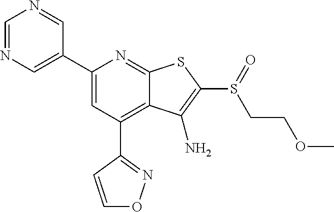 [SMILES: COCCS(=O)c1sc2nc(-c3cncnc3)cc(-c3ccon3)c2c1N] | *** |
| 353 | 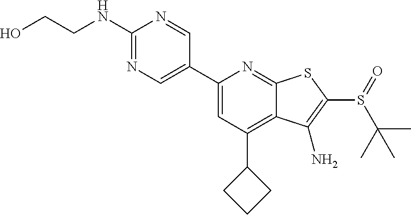 [SMILES: CC(C)(C)S(=O)c1sc2nc(-c3cnc(NCCO)nc3)cc(C3CCC3)c2c1N] | ** |
| 354 | 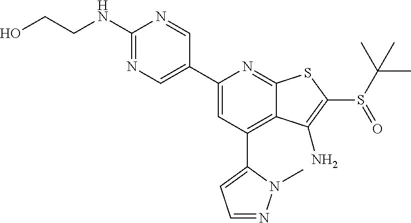 [SMILES: Cn1nccc1-c1cc(-c2cnc(NCCO)nc2)nc2sc(S(=O)C(C)(C)C)c(N)c12] | *** |
| 355 | 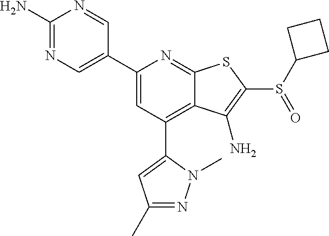 [SMILES: Cc1cc(-c2cc(-c3cnc(N)nc3)nc3sc(S(=O)C4CCC4)c(N)c23)n(C)n1] | *** |
| 356 | 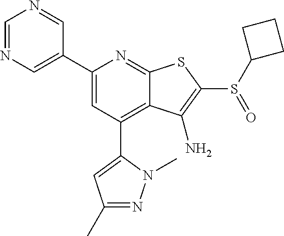 [SMILES: Cc1cc(-c2cc(-c3cncnc3)nc3sc(S(=O)C4CCC4)c(N)c23)n(C)n1] | *** |
| 357 | 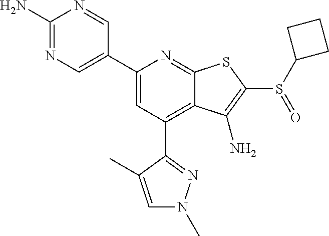 [SMILES: Cc1cn(C)nc1-c1cc(-c2cnc(N)nc2)nc2sc(S(=O)C3CCC3)c(N)c12] | *** |
| 358 | 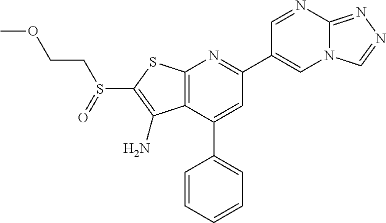 [SMILES: COCCS(=O)c1sc2nc(-c3cnc4nncn4c3)cc(-c3ccccc3)c2c1N] | * |
| 359 | 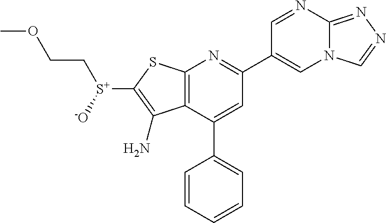 | *** |
| 360 | 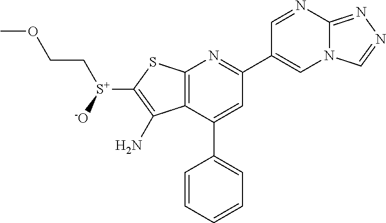 | * |
| 361 | 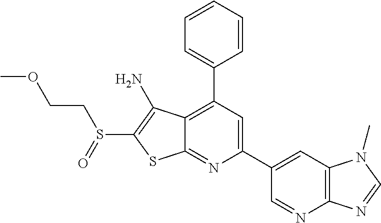 [SMILES: COCCS(=O)c1sc2nc(-c3cnc4ncn(C)c4c3)cc(-c3ccccc3)c2c1N] | *** |
| 362 | 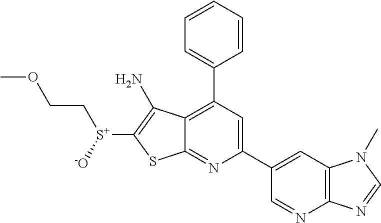 | *** |
| 363 | 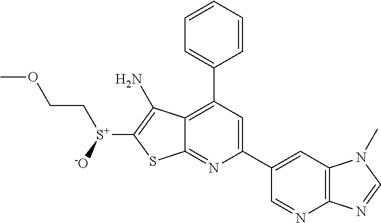 | * |
| 364 | 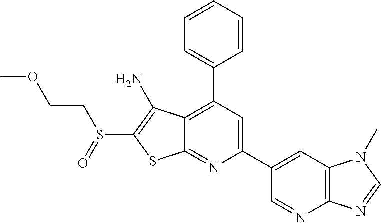 [SMILES: COCCS(=O)c1sc2nc(-c3cnc4ncn(C)c4c3)cc(-c3ccccc3)c2c1N] | *** |
| 365 | 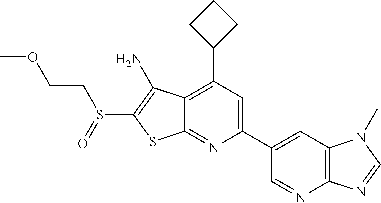 [SMILES: COCCS(=O)c1sc2nc(-c3cnc4ncn(C)c4c3)cc(C3CCC3)c2c1N] | *** |
| 366 | 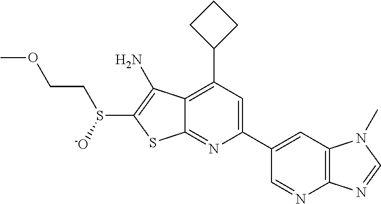 | *** |
| 367 | 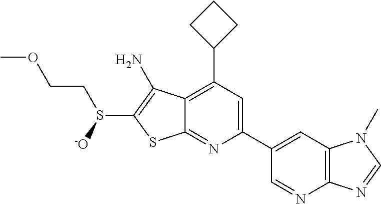 | * |
| 368 | 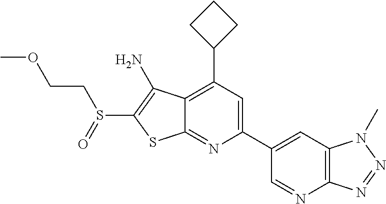 [SMILES: COCCS(=O)c1sc2nc(-c3cnc4nnn(C)c4c3)cc(C3CCC3)c2c1N] | *** |
| 369 | 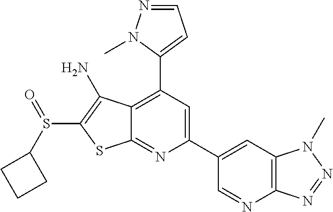 [SMILES: Cn1nccc1-c1cc(-c2cnc3nnn(C)c3c2)nc2sc(S(=O)C3CCC3)c(N)c12] | *** |
| 370 | 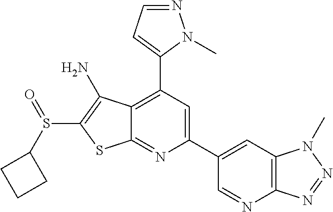 [SMILES: Cn1nccc1-c1cc(-c2cnc3nnn(C)c3c2)nc2sc(S(=O)C3CCC3)c(N)c12] | *** |
| 371 | 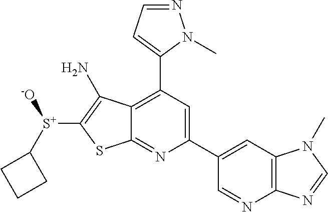 | *** |
| 372 | 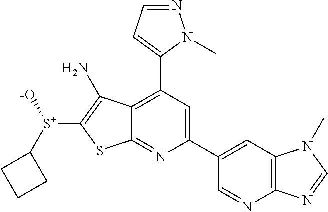 | * |
| 373 | 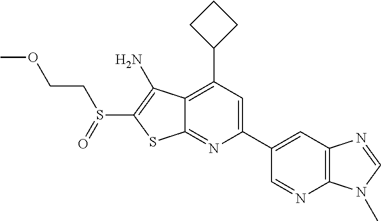 [SMILES: COCCS(=O)c1sc2nc(-c3cnc4c(c3)ncn4C)cc(C3CCC3)c2c1N] | *** |
| 374 | 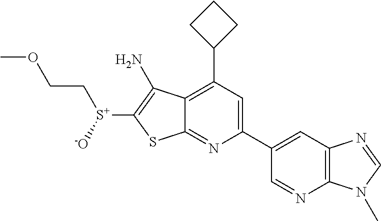 | *** |
| 375 | 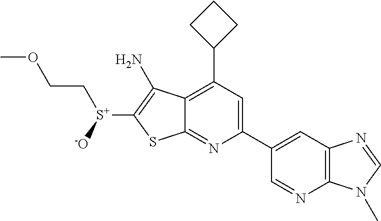 | * |
| 376 | 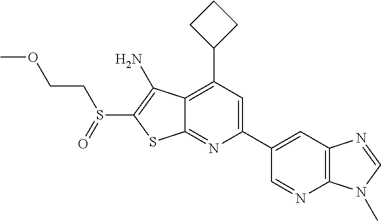 [SMILES: COCCS(=O)c1sc2nc(-c3cnc4c(c3)ncn4C)cc(C3CCC3)c2c1N] | *** |
| 377 | 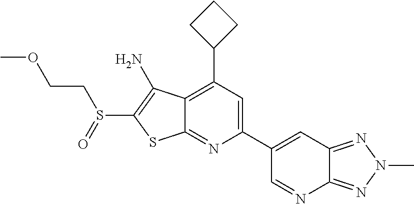 | *** |
| 378 | 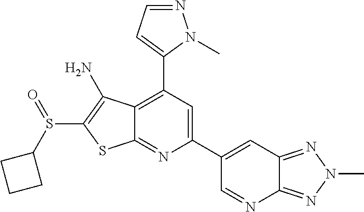 [SMILES: Cn1nc2cc(-c3cc(-c4ccnn4C)c4c(N)c(S(=O)C5CCC5)sc4n3)cnc2n1] | *** |
| 379 | 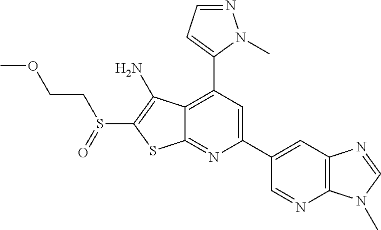 [SMILES: COCCS(=O)c1sc2nc(-c3cnc4c(c3)ncn4C)cc(-c3ccnn3C)c2c1N] | *** |
| 380 | 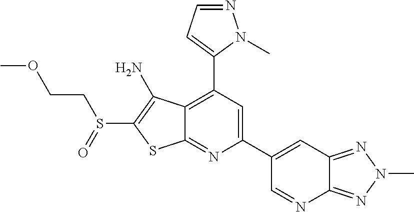 | *** |
| 381 | 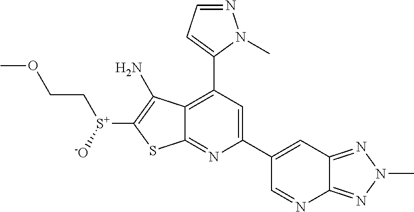 | *** |
| 382 | 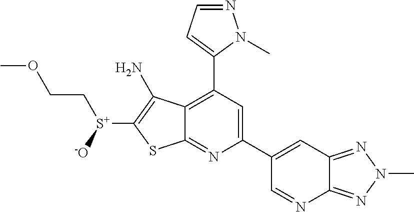 | * |
| 383 | 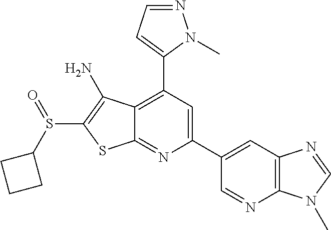 [SMILES: Cn1nccc1-c1cc(-c2cnc3c(c2)ncn3C)nc2sc(S(=O)C3CCC3)c(N)c12] | *** |
| 384 | 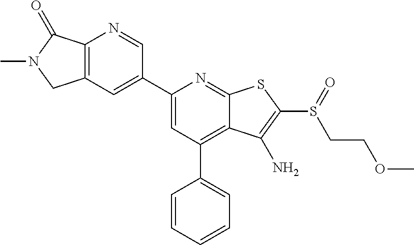 | *** |
| 385 | 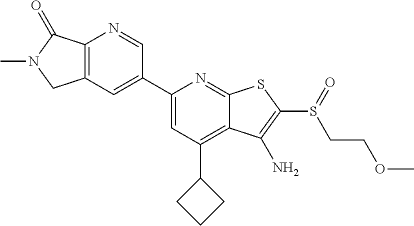 | *** |
| 386 | 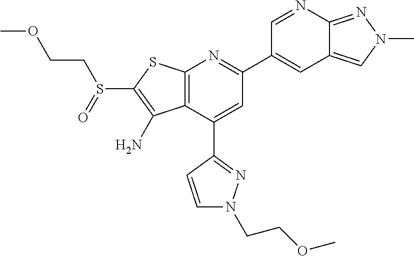 | *** |
| 387 | 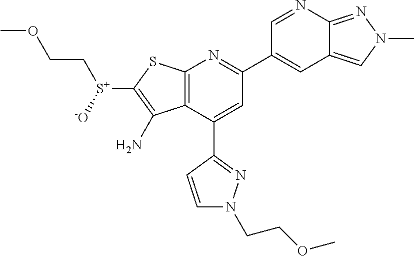 | *** |
| 388 | 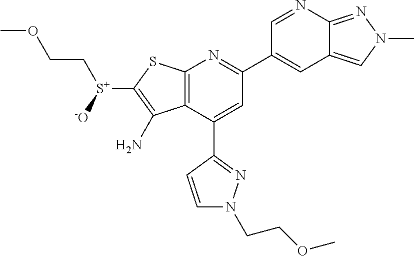 | * |
| 389 | 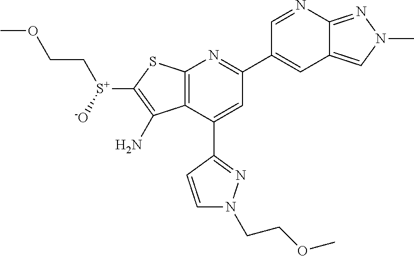 | *** |
| 390 | 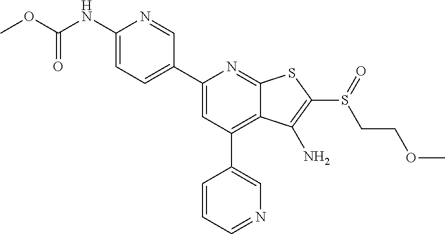 | ** |
| 391 | 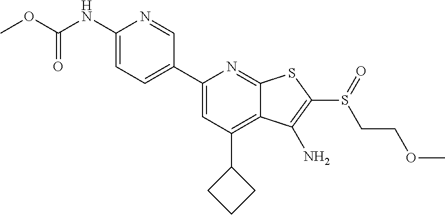 | ** |
| 392 | 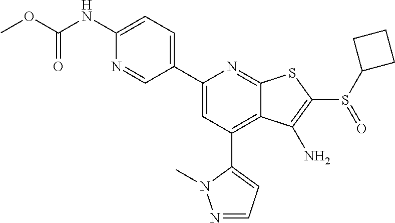 [SMILES: COC(=O)Nc1ccc(-c2cc(-c3ccnn3C)c3c(N)c(S(=O)C4CCC4)sc3n2)cn1] | *** |
| 393 | 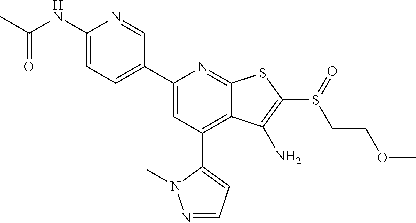 [SMILES: COCCS(=O)c1sc2nc(-c3ccc(NC(C)=O)nc3)cc(-c3ccnn3C)c2c1N] | ** |
| 394 | 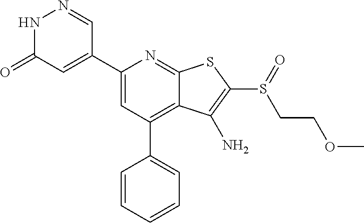 [SMILES: COCCS(=O)c1sc2nc(-c3cn[nH]c(=O)c3)cc(-c3ccccc3)c2c1N] | *** |
| 395 | 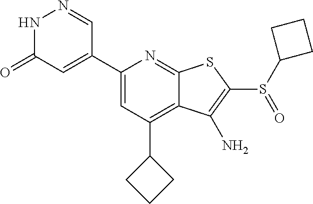 [SMILES: Nc1c(S(=O)C2CCC2)sc2nc(-c3cn[nH]c(=O)c3)cc(C3CCC3)c12] | *** |
| 396 | 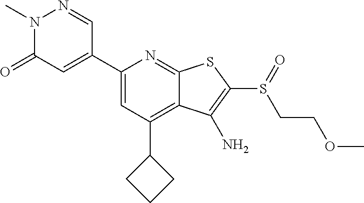 [SMILES: COCCS(=O)c1sc2nc(-c3cnn(C)c(=O)c3)cc(C3CCC3)c2c1N] | *** |
| 397 | 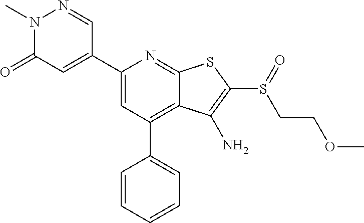 [SMILES: COCCS(=O)c1sc2nc(-c3cnn(C)c(=O)c3)cc(-c3ccccc3)c2c1N] | *** |
| 398 | 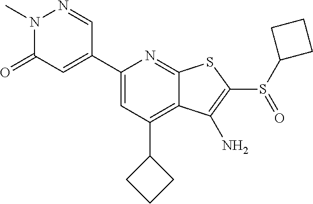 [SMILES: Cn1ncc(-c2cc(C3CCC3)c3c(N)c(S(=O)C4CCC4)sc3n2)cc1=O] | *** |
| 399 | 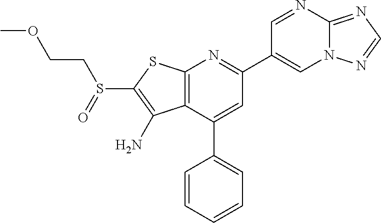 [SMILES: COCCS(=O)c1sc2nc(-c3cnc4ncnn4c3)cc(-c3ccccc3)c2c1N] | *** |
| 400 | 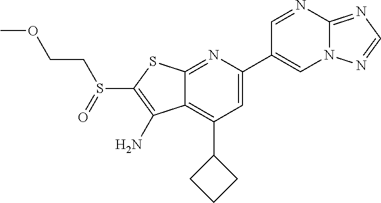 [SMILES: COCCS(=O)c1sc2nc(-c3cnc4ncnn4c3)cc(C3CCC3)c2c1N] | *** |
| 401 | 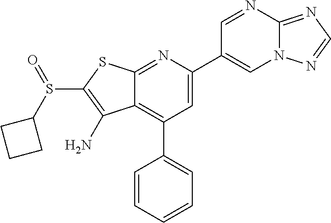 [SMILES: Nc1c(S(=O)C2CCC2)sc2nc(-c3cnc4ncnn4c3)cc(-c3ccccc3)c12] | *** |
| 402 | 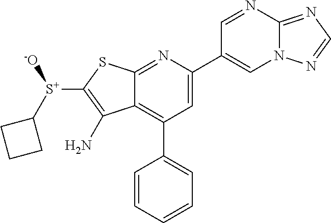 | *** |
| 403 | 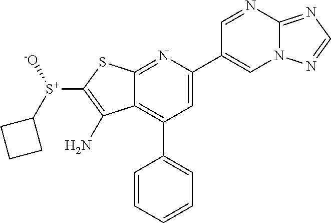 | * |
| 404 | 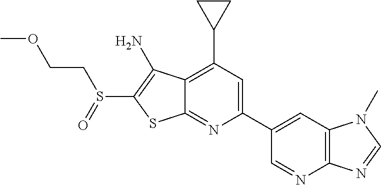 [SMILES: COCCS(=O)c1sc2nc(-c3cnc4ncn(C)c4c3)cc(C3CC3)c2c1N] | *** |
| 405 | 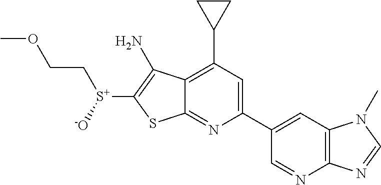 | *** |
| 406 | 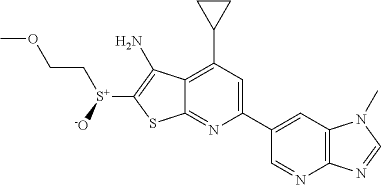 | * |
| 407 | 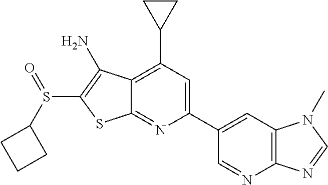 [SMILES: Cn1cnc2ncc(-c3cc(C4CC4)c4c(N)c(S(=O)C5CCC5)sc4n3)cc21] | *** |
| 408 | 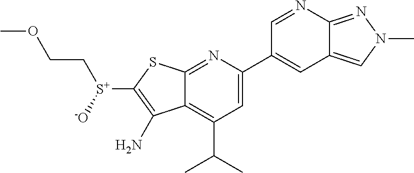 | *** |
| 409 | 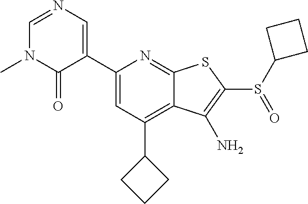 [SMILES: Cn1cncc(-c2cc(C3CCC3)c3c(N)c(S(=O)C4CCC4)sc3n2)c1=O] | *** |
| 410 | 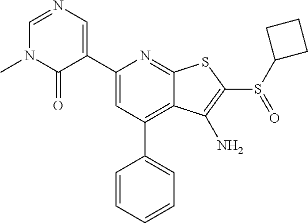 [SMILES: Cn1cncc(-c2cc(-c3ccccc3)c3c(N)c(S(=O)C4CCC4)sc3n2)c1=O] | ** |
| 411 | 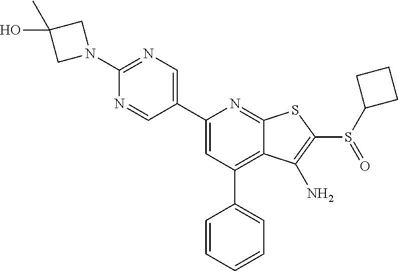 [SMILES: CC1(O)CN(c2ncc(-c3cc(-c4ccccc4)c4c(N)c(S(=O)C5CCC5)sc4n3)cn2)C1] | ** |
| 412 | 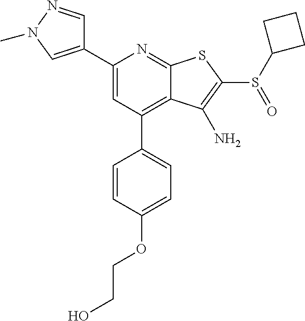 [SMILES: Cn1cc(-c2cc(-c3ccc(OCCO)cc3)c3c(N)c(S(=O)C4CCC4)sc3n2)cn1] | *** |
| 413 | 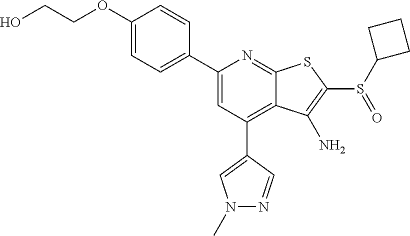 [SMILES: Cn1cc(-c2cc(-c3ccc(OCCO)cc3)nc3sc(S(=O)C4CCC4)c(N)c23)cn1] | *** |
| 414 | 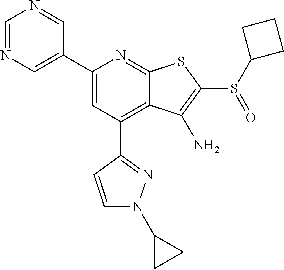 [SMILES: Nc1c(S(=O)C2CCC2)sc2nc(-c3cncnc3)cc(-c3ccn(C4CC4)n3)c12] | *** |
| 415 | 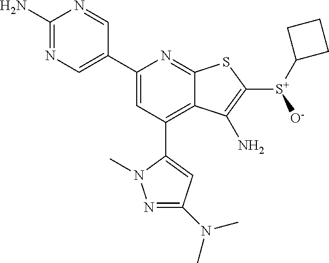 [SMILES: CN(C)c1cc(-c2cc(-c3cnc(N)nc3)nc3sc([S@+]([O-])C4CCC4)c(N)c23)n(C)n1] | *** |
| 416 | 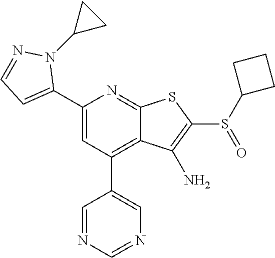 [SMILES: Nc1c(S(=O)C2CCC2)sc2nc(-c3ccnn3C3CC3)cc(-c3cncnc3)c12] | * |
| 417 | 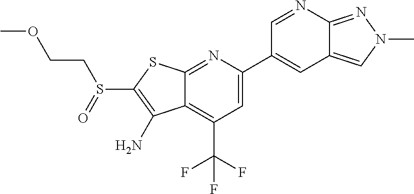 | *** |
| 418 | 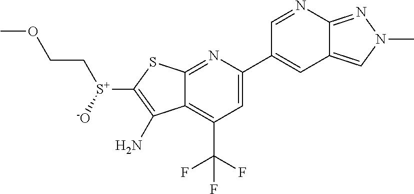 | *** |
| 419 | 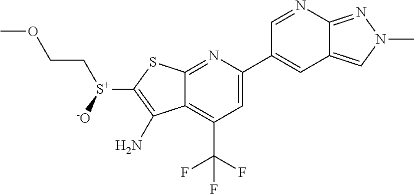 | * |
| 420 | 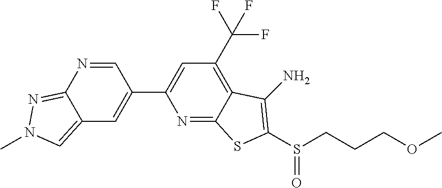 | *** |
| 421 | 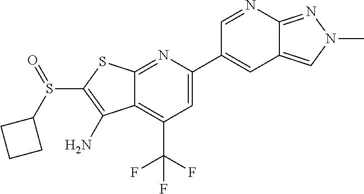 [SMILES: Cn1cc2cc(-c3cc(C(F)(F)F)c4c(N)c(S(=O)C5CCC5)sc4n3)cnc2n1] | *** |
| 422 | 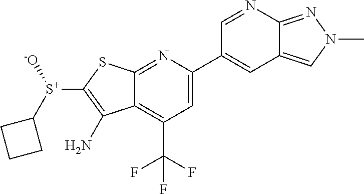 | * |
| 423 | 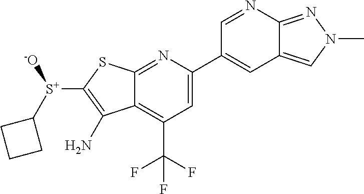 | *** |
| 424 | 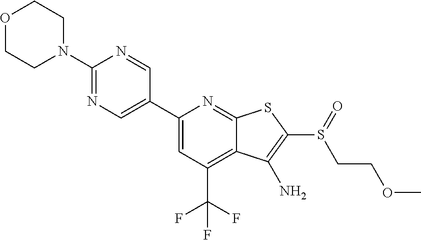 [SMILES: COCCS(=O)c1sc2nc(-c3cnc(N4CCOCC4)nc3)cc(C(F)(F)F)c2c1N] | *** |
| 425 | 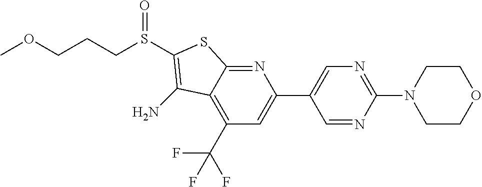 [SMILES: COCCCS(=O)c1sc2nc(-c3cnc(N4CCOCC4)nc3)cc(C(F)(F)F)c2c1N] | ** |
| 426 | 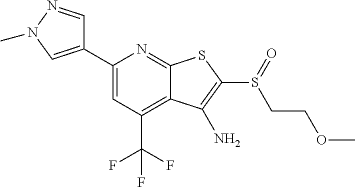 | *** |
| 427 | 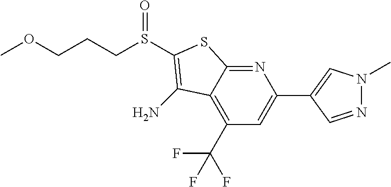 | *** |
| 428 | 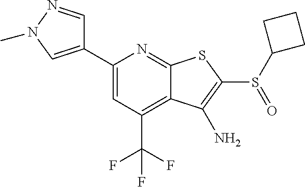 [SMILES: Cn1cc(-c2cc(C(F)(F)F)c3c(N)c(S(=O)C4CCC4)sc3n2)cn1] | *** |
| 429 | 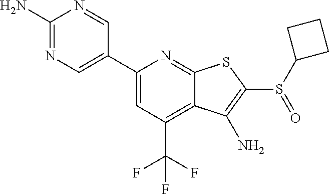 [SMILES: Nc1ncc(-c2cc(C(F)(F)F)c3c(N)c(S(=O)C4CCC4)sc3n2)cn1] | ** |
| 430 | 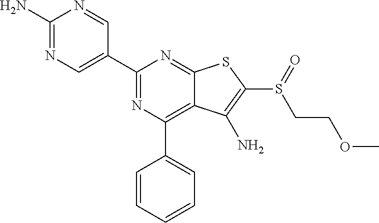 [SMILES: COCCS(=O)c1sc2nc(-c3cnc(N)nc3)nc(-c3ccccc3)c2c1N] | *** |
| 431 | 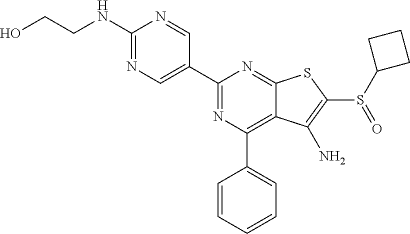 [SMILES: Nc1c(S(=O)C2CCC2)sc2nc(-c3cnc(NCCO)nc3)nc(-c3ccccc3)c12] | ** |
| 432 | 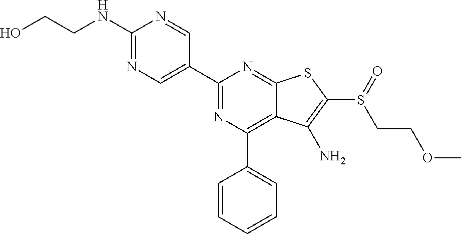 [SMILES: COCCS(=O)c1sc2nc(-c3cnc(NCCO)nc3)nc(-c3ccccc3)c2c1N] | ** |
| 433 | 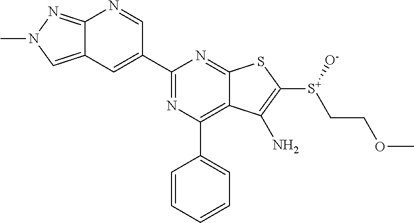 | *** |
| 434 | 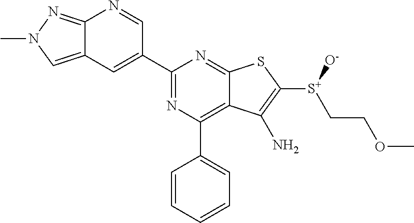 | * |

### Example B. Biological Assays

The activity (EC50 in nM) of the compounds was determined for induction of PGE2 from IL1-β treated A549 cells, as assayed in a medium of A549 cells that have been stimulated with by IL1-β for 24 hours. The metabolic stability was determined by incubating the compounds at a concentration of 10 μM in the presence of microsomes derived from mouse livers. Compound concentrations were monitored by HPLC/MS over time, and halflives were calculated from the slope of the best-fit line for Ln[compound] vs time.

Also, reference compounds and selected compounds described herein were given orally to mice at 10 mg/kg (mpk). Colon 15-PGDH activity at 30 minutes was assessed. As shown in Table 2, reference compound A was not sufficiently potent to inhibit colon 15-PGDH activity at 30 minutes and actually showed increased activity as indicated by the -55.2% inhibition. In comparison, compounds described herein demonstrated inhibition of column 15-PGDH activity at 30 minutes. Similar assays are run at 4 hours.

In Table 2, Reference Compounds A, B, and C have the following structures.

### Reference Compounds

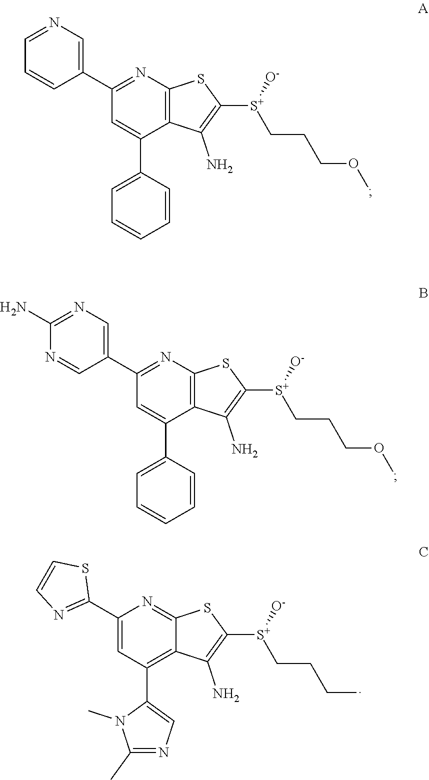

See WO2015/065716 and WO2018/218251 for disclosures of Reference Compounds A-C, the disclosures of each PCT applications are hereby incorporated by reference in their entireties for all purposes.

TABLE 2

| | Colon enzyme | Human | | Elevation of |
|---|---|---|---|---|
| | inhibition 30 min | microsome | hERG | PGE2 in A549 |
| Molecule No. (see | following 10 mpk | stability (T½, | IC₅₀ | cell culture media |
| Table 1)¹ | PO | min) | (μm) | (EC₅₀, nM) |
| Ref. Compd A | >70% but <90% | <60 | ≤15 | ≤10 |
| Ref. Compd B | >90% | <60 | ≤15 | ≤10 |
| Ref. Compd C | <70% | <60 | ≤15 | >10 |
| (R)-56 | <70% | ≥60 | >15 | ≤10 |
| (R)-57 | <70% | ≥60 | >15 | ≤10 |
| (R)-44 | >90% | ≥60 | >15 | ≤10 |
| (R)-55 | >90% | ≥60 | >15 | ≤10 |
| (R)-72 | >90% | ≥60 | >15 | ≤10 |
| (R)-73 | >90% | ≥60 | >15 | ≤10 |
| (R)-91 | <70% | ≥60 | >15 | >10 |
| (R)-110 | >90% | ≥60 | >15 | ≤10 |
| (R)-161 | >90% | ≥60 | >15 | ≤10 |
| (R)-195 | >70% but <90% | ≥60 | >15 | ≤10 |
| (R)-145 | >70% but <90% | ≥60 | >15 | ≤10 |
| (R)-224 | >90% | ≥60 | >15 | ≤10 |
| (R)-176 | >90% | ≥60 | >15 | ≤10 |
| (R)-126 | >70% but <90% | ≥60 | >15 | ≤10 |
| (R)-258 | >90% | ≥60 | >15 | ≤10 |
| (R)-326 | >70% but <90% | ≥60 | >15 | ≤10 |
| (R)-331 | N/A | ≥60 | >15 | ≤10 |
| (R)-381 | >90% | ≥60 | >15 | ≤10 |
| (R)-408 | >90% | ≥60 | >15 | ≤10 |

¹The indication “(R)” in front of the compound number indicates (R) isomer of the compound shown in Table 1. The stereoisomer indication is in relation to the sulfoxide sulfur atom.

The Applicants discovered the IC50 values, alone, are not predictive of in vivo efficacy, such as in disease models. Additional pharmacokinetic and pharmacodynamic properties, such as in vivo inhibition of 15-PGDH, human microsome stability, hERG IC₅₀, and EC50 for PGE2 elevation, are important to in vivo efficacy. Without being bound by theory, in certain embodiments, clinical candidates should have two or more of the following characteristics: (i) mouse colon enzyme inhibition 30 minutes following 10 MPK (PO) at ≥70% or ≥90%; (11) a human microsome stability ≥60 minutes; (111) hERG IC₅₀>15; and (iv) PGE₂ elevation EC50≤10 nM. In some embodiments, clinical candidates have three or more of characteristics (i), (ii), (iii), and (iv). In some embodiments, the clinical candidates have characteristics (i), (ii), (iii), and (iv). For example, a compound with a mouse colon enzyme inhibition 30 minutes following 10 MPK of ≥90%, or even ≥70%, may exhibit in vivo efficacy if such compound also demonstrates any combination of two or more of e.g., (ii) a human microsome stability of ≥60 minutes; (iii) hERG IC50 of >15; and/or (iiv) EC50 of ≤10 nM. Such compounds with in vivo efficacy are encompassed by the formula disclosed herein, e.g., Formula VIII.

In addition, the blood plasma concentrations of the compounds disclosed herein (e.g., the compounds of Formula VIII and/or Table 2) may be measured using known techniques, to determine, e.g., maximum blood concentrations, time to reach maximum blood concentrations, and area under the blood plasma concentration curve.

### Synthesis

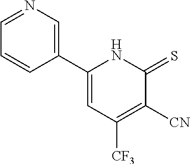 [SMILES: N#Cc1c(C(F)(F)F)cc(-c2cccnc2)[nH]c1=S]

Example 1A: 6-thioxo-4-(trifluoromethyl)-1,6-dihydro-[2,3′-bipyridine]-5-carbonitrile. Cyanothioacetamide (207 mg, 2.07 mmol) was added to a solution of 4,4,4-trifluoro-1-(pyridin-3-yl)butane-1,3-dione (300 mg, 1.38 mmol) and DABCO (155 mg, 1.38 mmol) in EtOH at room temperature. The reaction mixture was stirred under reflux. Once the reaction was completed, the mixture was allowed to stand overnight at 0° C., filtered and collected solid dried to give the title compound in quantitative yield. ESI-MS (m/z): 282.1 [M+H]⁺.

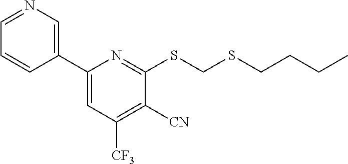 [SMILES: CCCCSCSc1nc(-c2cccnc2)cc(C(F)(F)F)c1C#N]

Example 1B: 6-(((butylthio)methyl)thio)-4-(trifluoromethyl)-[2,3′-bipyridine]-5-carbonitrile. A mixture of 6-thioxo-4-(trifluoromethyl)-1,6-dihydro-[2,3′-bipyridine]-5-carbonitrile (4.91 mmol, 1380 mg), butyl(chloromethyl)sulfane (4.91 mmol, 677 mg, 1.0 equiv) and Et₃N (7.36 mmol, 750 mg, 1.0 ml, 1.5 equiv) was stirred in dry CH₃CN (32.0 mL) for 20 min at 80° C. The reaction mixture was then diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 599 mg of desired product ¹H NMR (400 MHz, Chloroform-d) δ 9.32 (dt, J=2.4, 0.8 Hz, 1H), 8.79 (ddd, J=4.9, 1.7, 0.7 Hz, 1H), 8.39 (dddd, J=8.1, 2.4, 1.7, 0.6 Hz, 1H), 7.78 (s, 1H), 7.50 (ddt, J=8.1, 4.8, 0.8 Hz, 1H), 4.51 (s, 2H), 2.75 (t, J=7.8 Hz, 2H), 1.72-1.60 (m, 2H), 1.42 (h, J=7.4 Hz, 2H), 0.92 (t, J=7.3, 3H). ESI-MS (m/z): 384.1 [M+H]⁺.

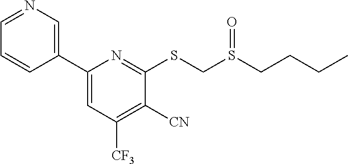 [SMILES: CCCCS(=O)CSc1nc(-c2cccnc2)cc(C(F)(F)F)c1C#N]

Example 1C: 6-(((butylsulfinyl)methyl)thio)-4-(trifluoromethyl)-[2,3′-bipyridine]-5-carbonitrile. To the solution of (((butylthio)methyl)thio)-4-(trifluoromethyl)-[2,3′-bipyridine]-5-carbonitrile (570 mg, 1.49 mmol) in CHCl₃/AcOH (1:1, 0.27 M) was added H₂O₂ (372 μL, 30% solution in water). The reaction mixture was stirred at 32° C. for 40 min. Once complete, the reaction was diluted with EtOAc and was washed with saturated NaHCO₃ solution, dried over magnesium sulfate, filtered and concentrated under reduce pressure to give 553 mg (93%) of desired compound. ¹H NMR (400 MHz, Chloroform-d) δ 9.28 (s, 1H), 8.80 (dd, J=4.8, 1.5 Hz, 1H), 8.49 (ddt, J=8.1, 2.6, 1.5 Hz, 1H), 7.87 (s, 1H), 7.55 (ddt, J=8.0, 4.9, 1.0 Hz, 1H), 4.71 (d, J=13.3 Hz, 1H), 4.54 (d, J=13.3 Hz, 1H), 3.00-2.80 (m, 2H), 1.90-1.75 (m, 2H), 1.61-1.37 (m, 2H), 0.96 (t, J=7.4, 3H). ESI-MS (m/z): 400.0 [M+H]⁺.

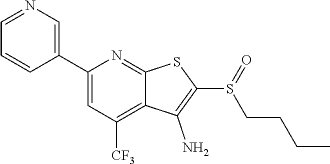 [SMILES: CCCCS(=O)c1sc2nc(-c3cccnc3)cc(C(F)(F)F)c2c1N]

EXAMPLE 1: 2-(butylsulfinyl)-6-(pyridin-3-yl)-4-(trifluoromethyl)thieno[2,3-b]pyridin-3-amine. To the solution of 6-(((butylsulfinyl)methyl)thio)-4-(trifluoromethyl)-[2,3′-bipyridine]-5-carbonitrile (410 mg, 1.03 mmol) in DMF (6.7 mL)/MeOH (3.35 mL) was added KOH (0.62 mmol, 34.7 mg, 347 μL in water). The reaction mixture was stirred at 32° C. for 10 min. Once complete, the reaction was diluted with EtOAc and acidified to pH 7 with 5% aq. solution of AcOH, the organic phase was separated and aqueous layer was extracted twice with EtOAc, dried over magnesium sulfate, filtered and concentrated under reduced pressure. The crude product was purified by flash chromatography to give desired product. ¹H NMR (400 MHz, Methylene Chloride-d₂) δ 9.33 (dd, J=2.4, 0.9 Hz, 1H), 8.72 (dd, J=4.8, 1.7 Hz, 1H), 8.46 (ddd, J=8.0, 2.4, 1.6 Hz, 1H), 8.11 (d, J=0.8 Hz, 1H), 7.49 (ddd, J=8.0, 4.8, 0.9 Hz, 1H), 5.24 (s, 2H), 3.33 (ddd, J=12.9, 9.0, 6.2 Hz, 1H), 3.17 (ddd, J=12.9, 9.1, 6.8 Hz, 1H), 1.90-1.68 (m, 2H), 1.52 (dd, J=7.3, 2.5 Hz, 2H), 0.97 (t, J=7.3 Hz, 3H). ESI-MS (m/z): 400.1 [M+H]⁺.

Enantiomers of EXAMPLE 1 were separated on a 1 cm Chiralpak AD-H column using 50% EtOH and 50% Hexanes with 5 mL/min flow rate, 400 μL injection (concentration 10 mg/ml) the 1st peak was at 7.8 min and the 2nd peak was at 15.1 min.

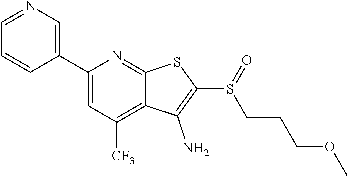 [SMILES: COCCCS(=O)c1sc2nc(-c3cccnc3)cc(C(F)(F)F)c2c1N]

EXAMPLE 2: 2-((3-methoxypropyl)sulfinyl)-6-(pyridin-3-yl)-4-(trifluoromethyl)thieno[2,3-b]pyridin-3-amine was prepared using the synthetic procedures described for the preparation of the analog EXAMPLE 1. ¹H NMR (500 MHz, Methylene Chloride-d₂) δ 9.37 (s, 1H), 8.76 (s, 1H), 8.50 (ddd, J=8.1, 2.3, 1.5 Hz, 1H), 8.14 (s, 1H), 7.53 (dd, J=8.0, 4.7 Hz, 1H), 5.27 (s, 2H), 3.56 (t, J=5.9 Hz, 2H), 3.41 (ddd, J=13.0, 8.4, 6.5 Hz, 1H), 3.37 (s, 3H), 3.30 (ddd, J=13.0, 8.4, 6.3 Hz, 1H), 2.19-1.96 (m, 2H). ESI-MS (m/z): 416.1 [M+H]⁺.

Enantiomers of EXAMPLE 2 were separated on a 2 cm Chiralpak AD-H column using 100% EtOH with 10 mL/min flow rate, 600 μL injection (concentration 8 mg/ml) the 1st peak was at 20 min and the 2nd peak was at 41 min.

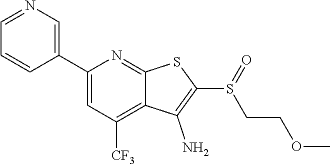 [SMILES: COCCS(=O)c1sc2nc(-c3cccnc3)cc(C(F)(F)F)c2c1N]

EXAMPLE 3: 2-((2-methoxyethyl)sulfinyl)-6-(pyridin-3-yl)-4-(trifluoromethyl)thieno[2,3-b]pyridin-3-amine was prepared using the synthetic procedures described for the preparation of the analog EXAMPLE 1. ¹H NMR (500 MHz, Methylene Chloride-d₂) δ 9.37 (s, 1H), 8.76 (d, J=4.3 Hz, 1H), 8.54-8.42 (m, 1H), 8.14 (s, 1H), 7.53 (ddd, J=8.0, 4.8, 0.9 Hz, 1H), 5.23 (s, 2H), 3.90 (ddd, J=9.9, 7.6, 3.7 Hz, 1H), 3.71 (ddd, J=10.1, 6.3, 4.0 Hz, 1H), 3.66 (ddd, J=12.9, 6.3, 3.7 Hz, 1H), 3.42 (s, 3H), 3.35 (ddd, J=12.8, 7.6, 4.0 Hz, 1H). ESI-MS (m/z): 402.1. [M+H]⁺.

Enantiomers of EXAMPLE 3 were separated on a 2 cm Chiralpak AD-H column using 80% EtOH and 20% Hexanes with 10 m/min flow rate, 600 μL injection (concentration 8 mg/ml) the 1st peak was at 21.9 min and the 2nd peak was at 30 min.

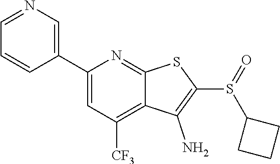 [SMILES: Nc1c(S(=O)C2CCC2)sc2nc(-c3cccnc3)cc(C(F)(F)F)c12]

EXAMPLE 4: 2-(cyclobutylsulfinyl)-6-(pyridin-3-yl)-4-(trifluoromethyl)thieno[2,3-b]pyridin-3-amine was prepared using the synthetic procedures described for the preparation of the analog EXAMPLE 1. ¹H NMR (400 MHz, Methylene Chloride-d₂) δ 9.33 (s, 1H), 8.72 (s, 1H), 8.59-8.37 (m, 1H), 8.09 (s, 1H), 7.49 (dd, J=8.0, 4.8 Hz, 1H), 5.18 (s, 2H), 4.04-3.90 (m, 1H), 2.89-2.71 (m, 1H), 2.51-2.36 (m, 1H), 2.36-2.19 (m, 2H), 2.18-1.99 (m, 2H). ESI-MS (m/z): 398.1 [M+H]⁺.

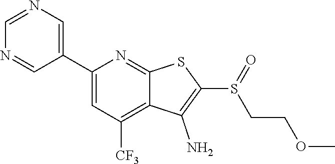 [SMILES: COCCS(=O)c1sc2nc(-c3cncnc3)cc(C(F)(F)F)c2c1N]

EXAMPLE 5: 2-((2-methoxyethyl)sulfinyl)-6-(pyrimidin-5-yl)-4-(trifluoromethyl)thieno[2,3-b]pyridin-3-amine was prepared using the synthetic procedures described for the preparation of the analog EXAMPLE 1. ¹H NMR (400 MHz, Methylene Chloride-d₂) δ 9.46 (s, 2H), 9.30 (s, 1H), 8.10 (s, 1H), 5.21 (s, 2H), 3.87 (ddd, J=9.4, 7.7, 3.5 Hz, 1H), 3.73-3.59 (m, 2H), 3.39 (s, 3H), 3.32 (ddd, J=12.6, 7.6, 3.6 Hz, 1H). ESI-MS (m/z): 403.0 [M+H]⁺.

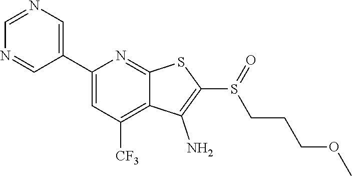 [SMILES: COCCCS(=O)c1sc2nc(-c3cncnc3)cc(C(F)(F)F)c2c1N]

EXAMPLE 6: 2-((3-methoxypropyl)sulfinyl)-6-(pyrimidin-5-yl)-4-(trifluoromethyl)thieno[2,3-b]pyridin-3-amine was prepared using the synthetic procedures described for the preparation of the analog EXAMPLE 1. ¹H NMR (400 MHz, Methylene Chloride-d₂) δ 9.46 (s, 2H), 9.30 (s, 1H), 7.97 (s, 1H), 5.26 (s, 2H), 3.53 (t, J=5.9 Hz, 2H), 3.42-3.35 (m, 1H), 3.34 (s, 3H), 3.27 (ddd, J=13.0, 8.3, 6.3 Hz, 1H), 2.17-1.91 (m, 2H). ESI-MS (m/z): 417.1 [M+H]⁺.

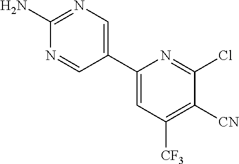 [SMILES: N#Cc1c(C(F)(F)F)cc(-c2cnc(N)nc2)nc1Cl]

Example 7A: 6-(2-aminopyrimidin-5-yl)-2-chloro-4-(trifluoromethyl)nicotinonitrile. To the solution of 2,6-dichloro-4-(trifluoromethyl)nicotinonitrile (100 mg, 0.416 mmol), (2-aminopyrimidin-5-yl)boronic acid (0.39 mmol, 55.0 mg, 0.95 equiv), [1,1′-bis(diphenylphosphino)ferrocene]dichloropalladium(II) (0.019 mmol, 16 mg, 5 mol %) in degassed dioxane (1.0 mL) was added degassed 2M aqueous solution of sodium carbonate (0.5 mL) and the reaction mixture was stirred for 2.5 h under nitrogen at 100° C. Once complete, the reaction was diluted with EtOAc and water. The organic phase was separated and aqueous layer was extracted twice with EtOAc, dried over magnesium sulfate, filtered and concentrated under reduced pressure to give crude product, which was purified by flash column chromatography in 51% isolated yield (64 mg). ESI-MS (m/z): 300.0 [M+H]⁺.

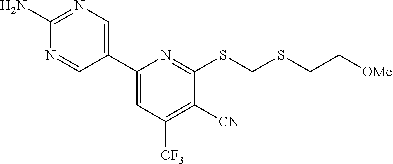 [SMILES: COCCSCSc1nc(-c2cnc(N)nc2)cc(C(F)(F)F)c1C#N]

Example 7B: 6-(2-aminopyrimidin-5-yl)-2-((((2-methoxyethyl)thio)methyl)thio)-4-(trifluoromethyl)nicotinonitrile. To the solution of 6-(2-aminopyrimidin-5-yl)-2-chloro-4-(trifluoromethyl)nicotinonitrile (64 mg, 0.214 mmol) in DMF (200 μL) was added sodium sulfide (18.3 mg, 0.24 mmol, 1.1 equiv) and the reaction mixture was stirred at 100° C. for 20 min. The progress of the reaction was followed by LCMS. Once complete (about 20 min), conc. HCl was added and the reaction mixture was stirred in the hood for 10 min. ESI-MS (m/z): 298.0 [M+H]⁺.

The reaction mixture was diluted with CH₃CN (1.50 mL). Et₃N (0.65 mmol, 65.6 mg, 90 μL. 3.0 equiv) was added followed by (chloromethyl)(2-methoxyethyl)sulfane (0.428 mmol, 60 mg, 2.0 equiv). The reaction mixture was stirred at room temperature for 15 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 24 mg of product (28%). ¹H NMR (400 MHz, Chloroform-d) δ 9.03 (s, 2H), 7.59 (s, 1H), 5.52 (s, 2H), 4.55 (s, 2H), 3.67 (t, J=6.0 Hz, 2H), 3.38 (s, 3H), 2.90 (t, J=6.0 Hz, 2H). ESI-MS (m/z): 402.1 [M+H]⁺.

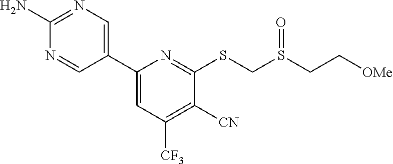 [SMILES: COCCS(=O)CSc1nc(-c2cnc(N)nc2)cc(C(F)(F)F)c1C#N]

Example 7C: 6-(2-aminopyrimidin-5-yl)-2-((((2-methoxyethyl)sulfinyl)methyl)thio)-4-(trifluoromethyl)nicotinonitrile was prepared via standard oxidation with hydrogen peroxide in 99% isolated yield, using synthetic procedures described for the preparation of the analog EXAMPLE 1. ¹H NMR (400 MHz, Chloroform-d) δ 9.03 (s, 2H), 7.65 (s, 1H), 6.15 (s, 2H), 4.78 (d, J=13.2 Hz, 1H), 4.67 (d, J=13.1 Hz, 1H), 3.98 (ddd, J=10.7, 6.0, 3.6 Hz, 1H), 3.80 (ddd, J=11.0, 8.4, 3.1 Hz, 1H), 3.40 (s, 3H), 3.18 (ddd, J=13.8, 8.5, 3.7 Hz, 1H), 3.07 (ddd, J=13.8, 6.0, 3.1 Hz, 1H). ESI-MS (m/z): 418.1 [M+H]⁺.

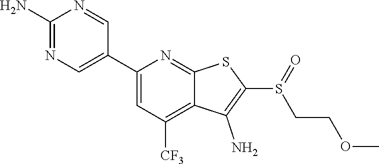 [SMILES: COCCS(=O)c1sc2nc(-c3cnc(N)nc3)cc(C(F)(F)F)c2c1N]

EXAMPLE 7: 6-(2-aminopyrimidin-5-yl)-2-((2-methoxyethyl)sulfinyl)-4-(trifluoromethyl)thieno[2,3-b]pyridin-3-amine was prepared in 71% isolated yield via standard cyclization reaction with potassium hydroxide, using the synthetic procedures described for the preparation of the analog EXAMPLE 1. ¹H NMR (400 MHz, Acetone-d₆) δ 9.14 (s, 2H), 8.25 (s, 1H), 6.59 (s, 2H), 5.40 (s, 2H), 3.85 (ddd, J=10.7, 7.9, 4.2 Hz, 1H), 3.70 (ddd, J=10.5, 5.8, 4.5 Hz, 1H), 3.52 (ddd, J=13.2, 5.9, 4.2 Hz, 1H), 3.35 (s, 3H), 3.33-3.27 (m, 1H). ESI-MS (m/z): 418.1 [M+H]⁺.

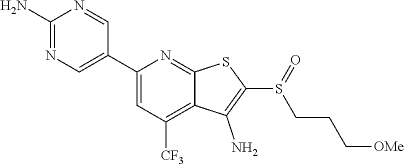 [SMILES: COCCCS(=O)c1sc2nc(-c3cnc(N)nc3)cc(C(F)(F)F)c2c1N]

EXAMPLE 8: 6-(2-aminopyrimidin-5-yl)-2-((3-methoxypropyl)sulfinyl)-4-(trifluoromethyl)thieno[2,3-b]pyridin-3-amine was prepared using the synthetic procedures described for the preparation of the analog EXAMPLE 7. ¹H NMR (400 MHz, Methylene Chloride-d₂) δ 9.06 (s, 2H), 7.93 (s, 1H), 5.46 (s, 2H), 5.20 (s, 2H), 3.52 (t, J=5.9 Hz, 2H), 3.41-3.34 (m, 1H), 3.34 (s, 3H), 3.25 (ddd, J=13.2, 8.3, 6.5 Hz, 1H), 2.16-1.88 (m, 2H). ESI-MS (m/z): 432.1 [M+H]⁺.

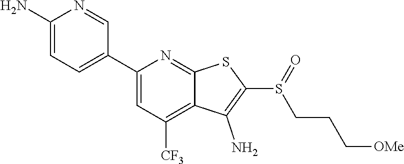 [SMILES: COCCCS(=O)c1sc2nc(-c3ccc(N)nc3)cc(C(F)(F)F)c2c1N]

EXAMPLE 9: 6-(6-aminopyridin-3-yl)-2-((3-methoxypropyl)sulfinyl)-4-(trifluoromethyl)thieno[2,3-b]pyridin-3-amine was prepared using the synthetic procedures described for the preparation of the analog EXAMPLE 7. ¹H NMR (400 MHz, Methylene Chloride-d₂) δ 8.81 (s, 1H), 8.26 (dd, J=8.7, 2.4 Hz, 1H), 7.96 (s, 1H), 6.66 (d, J=9.0 Hz, 1H), 5.18 (s, 2H), 5.03 (s, 2H), 3.52 (t, J=5.9 Hz, 2H), 3.43-3.35 (m, 1H), 3.33 (s, 3H), 3.29-3.18 (m, 1H), 2.05-1.96 (m, 2H). ESI-MS (m/z): 431.1 [M+H]⁺.

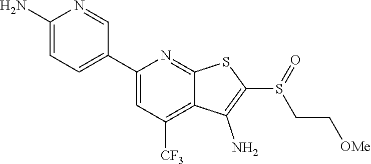 [SMILES: COCCS(=O)c1sc2nc(-c3ccc(N)nc3)cc(C(F)(F)F)c2c1N]

EXAMPLE 10: 6-(6-aminopyridin-3-yl)-2-((2-methoxyethyl)sulfinyl)-4-(trifluoromethyl)thieno[2,3-b]pyridin-3-amine was prepared using the synthetic procedures described for the preparation of the analog EXAMPLE 7. ¹H NMR (400 MHz, Methylene Chloride-d₂) δ 8.82 (s, 1H), 8.26 (dd, J=8.9, 2.3 Hz, 1H), 7.97 (s, 1H), 6.66 (d, J=8.7 Hz, 1H), 5.14 (s, 2H), 4.97 (s, 2H), 3.90-3.80 (m, 1H), 3.71-3.56 (m, 2H), 3.38 (s, 3H), 3.33-3.24 (m, 1H). ESI-MS (m/z): 417.1 [M+H]⁺.

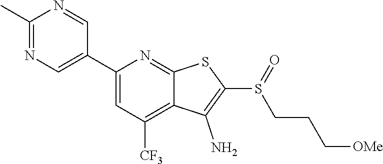 [SMILES: COCCCS(=O)c1sc2nc(-c3cnc(C)nc3)cc(C(F)(F)F)c2c1N]

EXAMPLE 11: 2-((3-methoxypropyl)sulfinyl)-6-(2-methylpyrimidin-5-yl)-4-(trifluoromethyl)thieno[2,3-b]pyridin-3-amine was prepared using the synthetic procedures described for the preparation of the analog EXAMPLE 7. ¹H NMR (400 MHz, Methylene Chloride-d₂) δ 9.35 (s, 2H), 8.06 (s, 1H), 5.25 (s, 2H), 3.52 (t, J=5.9 Hz, 2H), 3.42-3.35 (m, 1H), 3.34 (s, 3H), 3.30-3.22 (m, 1H), 2.80 (s, 3H), 2.11-1.90 (m, 2H). ESI-MS (m/z): 431.1 [M+H]⁺.

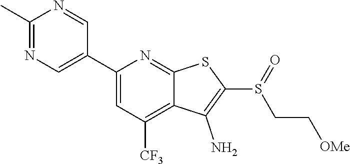 [SMILES: COCCS(=O)c1sc2nc(-c3cnc(C)nc3)cc(C(F)(F)F)c2c1N]

EXAMPLE 12: 2-((2-methoxyethyl)sulfinyl)-6-(2-methylpyrimidin-5-yl)-4-(trifluoromethyl)thieno[2,3-b]pyridin-3-amine was prepared using the synthetic procedures described for the preparation of the analog EXAMPLE 7. ¹H NMR (400 MHz, Methylene Chloride-d₂) δ 9.35 (s, 2H), 8.06 (d, J=1.2 Hz, 1H), 5.20 (s, 2H), 3.87 (ddd, J=9.5, 7.6, 3.4 Hz, 1H), 3.73-3.58 (m, 2H), 3.39 (s, 3H), 3.32 (ddd, J=12.9, 7.6, 3.6 Hz, 1H), 2.81 (s, 3H). ESI-MS (m/z): 417.0 [M+H]⁺.

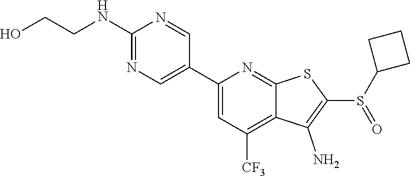 [SMILES: Nc1c(S(=O)C2CCC2)sc2nc(-c3cnc(NCCO)nc3)cc(C(F)(F)F)c12]

EXAMPLE 13: 2-((5-(3-amino-2-(cyclobutylsulfinyl)-4-(trifluoromethyl)thieno[2,3-b]pyridin-6-yl)pyrimidin-2-yl)amino)ethan-1-ol was prepared using the synthetic procedures described for the preparation of the analog EXAMPLE 7. ¹H NMR (500 MHz, Methylene Chloride-d₂) δ 9.06 (s, 2H), 7.92 (s, 1H), 6.03 (s, 1H), 5.17 (s, 2H), 4.06-3.95 (m, 1H), 3.88 (t, J=5.5 Hz, 2H), 3.71 (td, J=5.7, 3.0 Hz, 2H), 2.90-2.77 (m, 1H), 2.50-2.37 (m, 1H), 2.37-2.30 (m, 1H), 2.30-2.22 (m, 1H), 2.17-2.04 (m, 2H). ESI-MS (m/z): 458.1 [M+H]⁺.

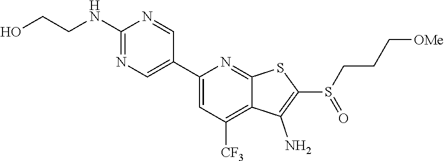 [SMILES: COCCCS(=O)c1sc2nc(-c3cnc(NCCO)nc3)cc(C(F)(F)F)c2c1N]

EXAMPLE 14: 2-((5-(3-amino-2-((3-methoxypropyl)sulfinyl)-4-(trifluoromethyl)thieno[2,3-b]pyridin-6-yl)pyrimidin-2-yl)amino)ethan-1-ol was prepared using the synthetic procedures described for the preparation of the analog EXAMPLE 7. ¹H NMR (400 MHz, Methylene Chloride-d₂) δ 9.03 (s, 2H), 7.89 (s, 1H), 6.01 (s, 1H), 5.19 (s, 2H), 3.95-3.79 (m, 2H), 3.68 (td, J=5.7, 4.4 Hz, 2H), 3.52 (t, J=5.9 Hz, 2H), 3.41-3.31 (m, 1H), 3.33 (s, 3H), 3.24 (ddd, J=13.0, 8.2, 6.5 Hz, 1H), 3.00 (s, 1H), 2.18-1.86 (m, 2H). ESI-MS (m/z): 476.1 [M+H]⁺.

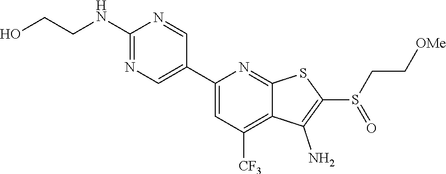 [SMILES: COCCS(=O)c1sc2nc(-c3cnc(NCCO)nc3)cc(C(F)(F)F)c2c1N]

EXAMPLE 15: 2-((5-(3-amino-2-((2-methoxyethyl)sulfinyl)-4-(trifluoromethyl)thieno[2,3-b]pyridin-6-yl)pyrimidin-2-yl)amino)ethan-1-ol was prepared using the synthetic procedures described for the preparation of the analog EXAMPLE 7. ¹H NMR (400 MHz, Methylene Chloride-d₂) δ 9.05 (s, 2H), 7.97 (s, 1H), 6.16 (s, 1H), 5.15 (s, 2H), 3.90-3.78 (m, 3H), 3.73-3.53 (m, 4H), 3.38 (s, 3H), 3.30 (ddd, J=12.1, 7.5, 3.7 Hz, 1H). ESI-MS (m/z): 462.1 [M+H]⁺.

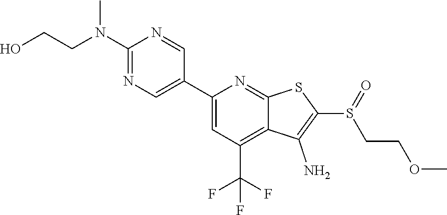 [SMILES: COCCS(=O)c1sc2nc(-c3cnc(N(C)CCO)nc3)cc(C(F)(F)F)c2c1N]

EXAMPLE 16: 2-((5-(3-amino-2-((2-methoxyethyl)sulfinyl)-4-(trifluoromethyl)thieno[2,3-b]pyridin-6-yl)pyrimidin-2-yl)(methyl)amino)ethan-1-ol was prepared using the synthetic procedures described for the preparation of the analog EXAMPLE 7. ¹H NMR (400 MHz, Methylene Chloride-d₂) δ 9.05 (s, 2H), 7.90 (s, 1H), 5.14 (s, 2H), 3.88 (s, 4H), 3.87-3.81 (m, 1H), 3.71-3.56 (m, 2H), 3.38 (s, 3H), 3.32 (s, 3H), 3.31-3.24 (m, 1H). ESI-MS (m/z): 476.1 [M+H]⁺.

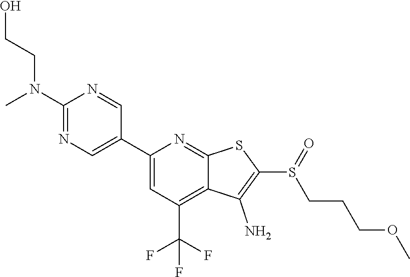

EXAMPLE 17: 2-((5-(3-amino-2-((3-methoxypropyl)sulfinyl)-4-(trifluoromethyl)thieno[2,3-b]pyridin-6-yl)pyrimidin-2-yl)(methyl)amino)ethan-1-ol was prepared using the synthetic procedures described for the preparation of the analog EXAMPLE 7. ¹H NMR (400 MHz, Methylene Chloride-d₂) δ 9.06 (s, 2H), 7.90 (s, 1H), 5.19 (s, 2H), 3.89 (s, 4H), 3.51 (t, J=5.9 Hz, 2H), 3.40-3.34 (m, 1H), 3.33 (s, 3H), 3.32 (s, 3H), 3.24 (ddd, J=13.1, 8.3, 6.6 Hz, 1H), 2.18-1.86 (m, 2H). ESI-MS (m/z): 490.1 [M+H]⁺.

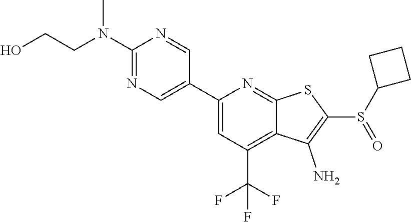 [SMILES: CN(CCO)c1ncc(-c2cc(C(F)(F)F)c3c(N)c(S(=O)C4CCC4)sc3n2)cn1]

EXAMPLE 18: 2-((5-(3-amino-2-(cyclobutylsulfinyl)-4-(trifluoromethyl)thieno[2,3-b]pyridin-6-yl)pyrimidin-2-yl)(methyl)amino)ethan-1-ol was prepared using the synthetic procedures described for the preparation of the analog EXAMPLE 7. ¹H NMR (400 MHz, Methylene Chloride-d₂) δ 9.04 (s, 2H), 7.89 (s, 1H), 5.13 (s, 2H), 3.97 (p, J=8.1 Hz, 1H), 3.88 (s, 4H), 3.32 (s, 3H), 2.87-2.73 (m, 1H), 2.45-2.35 (m, 1H), 2.35-2.17 (m, 2H), 2.15-1.93 (m, 2H). ESI-MS (m/z): 472.1 [M+H]⁺.

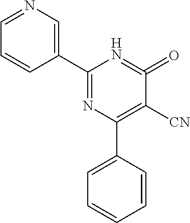

Example 19A: 6-oxo-4-phenyl-2-(pyridin-3-yl)-1,6-dihydropyrimidine-5-carbonitrile. The solution of benzaldehyde (1.00 g, 9.4 mmol), ethyl cyanoacetate (1064 mg, 9.4 mmol) and catalytic amount of piperidine in MeOH (15 mL) was stirred at room temperature overnight. The solvent was evaporated to give the desired product in quantitative yield.

The crude product (3.0 mmol, 603 mg) was dissolved in EtOH (8 mL), nicotinimidamide hydrochloride (1.5 equiv, 4.5 mmol, 706 mg) and potassium carbonate (3.0 equiv, 9 mmol, 1.24 g) were added and the reaction mixture was stirred at 80° C. overnight. Once completed, the reaction mixture was filtered, the obtained solid suspended in water, filtered and dried to give 6-oxo-4-phenyl-2-(pyridin-3-yl)-1,6-dihydropyrimidine-5-carbonitrile (260 mg). ESI-MS (m/z): 275.1 [M+H]⁺.

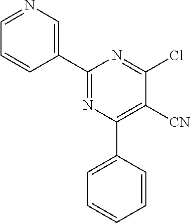 [SMILES: N#Cc1c(Cl)nc(-c2cccnc2)nc1-c1ccccc1]

Example 19B: 4-chloro-6-phenyl-2-(pyridin-3-yl)pyrimidine-5-carbonitrile. The reaction mixture of 6-oxo-4-phenyl-2-(pyridin-3-yl)-1,6-dihydropyrimidine-5-carbonitrile in POCl3 (1 mL) was stirred at 100° C. for 20 min. Once completed (the reaction progress was monitored by LCMS) the reaction mixture was cooled to room temperature and Et₂₀ was added. The formed solid/oil was separated from the liquid, dried and used in the next step without further purification. ESI-MS (m/z): 293.1 [M+H]⁺.

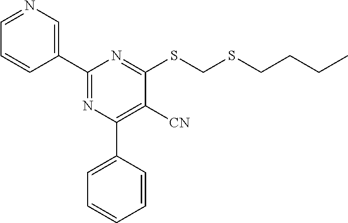 [SMILES: CCCCSCSc1nc(-c2cccnc2)nc(-c2ccccc2)c1C#N]

Example 19C: 4-(((butylthio)methyl)thio)-6-phenyl-2-(pyridin-3-yl)pyrimidine-5-carbonitrile. To the crude 4-(((butylthio)methyl)thio)-6-phenyl-2-(pyridin-3-yl)pyrimidine-5-carbonitrile (300 mg, 1.0 mmol) in DMF (1 mL) was added sodium sulfide (85 mg, 1.09 mmol, 1.09 equiv) and the reaction mixture was stirred at 100° C. for 20 min. The progress of the reaction was followed by LCMS. Once complete, conc. HCl was added and the reaction mixture was stirred in the hood for 10 min. ESI-MS (m/z): 291.0 [M+H]⁺.

The reaction mixture was diluted with CH₃CN (3 mL). Et₃N (500 mg, 4.95 mmol) was added followed by butyl(chloromethyl)sulfane (3.0 mmol, 414 mg). The reaction mixture was stirred at room temperature for 15 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 243 mg of product (62%). ¹H NMR (400 MHz, Chloroform-d) δ 9.83 (s, 1H), 9.43 (d, J=8.8 Hz, 1H), 9.03 (s, 1H), 8.28-8.02 (m, 3H), 7.75-7.47 (m, 3H), 4.53 (s, 2H), 2.76 (t, J=7.4 Hz, 2H), 1.64 (p, J=7.4 Hz, 2H), 1.43 (h, J=7.3 Hz, 2H), 0.92 (t, J=7.4 Hz, 3H). ESI-MS (m/z): 393.1 [M+H]⁺.

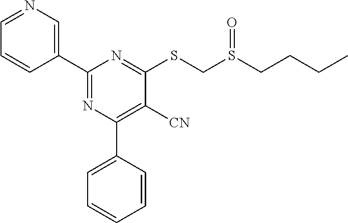 [SMILES: CCCCS(=O)CSc1nc(-c2cccnc2)nc(-c2ccccc2)c1C#N]

Example 19D: 4-(((butylsulfinyl)methyl)thio)-6-phenyl-2-(pyridin-3-yl)pyrimidine-5-carbonitrile was prepared via standard oxidation with hydrogen peroxide in 98% isolated yield, using synthetic procedures described for the preparation of the analog EXAMPLE 1. ¹H NMR (400 MHz, Chloroform-d) δ 9.76 (s, 1H), 8.86 (d, J=8.1 Hz, ¹H), 8.82 (d, J=6.9 Hz, 1H), 8.19-8.12 (m, 2H), 7.68-7.56 (m, 4H), 4.77 (d, J=13.2 Hz, 1H), 4.59 (d, J=13.3 Hz, 1H), 3.05-2.85 (m, 2H), 1.95-1.74 (m, 2H), 1.64-1.43 (m, 2H), 0.97 (t, J=7.3 Hz, 3H). ESI-MS (m/z): 409.1 [M+H]⁺.

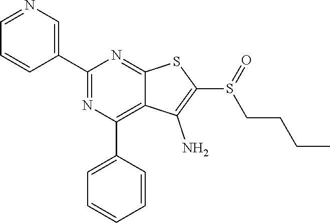 [SMILES: CCCCS(=O)c1sc2nc(-c3cccnc3)nc(-c3ccccc3)c2c1N]

EXAMPLE 19: 6-(butylsulfinyl)-4-phenyl-2-(pyridin-3-yl)thieno[2,3-d]pyrimidin-5-amine. To the solution of 6-(butylsulfinyl)-4-phenyl-2-(pyridin-3-yl)thieno[2,3-d]pyrimidin-5-amine (0.47 mmol, 193 mg) in DMF (3.0 mL) was added KOH (0.35 mmol, 20 mg, 0.75 equiv. in 200 μl of water). The reaction mixture was stirred at r.t. for 20 min (the reaction was monitored by TLC). Once complete, the reaction was diluted with EtOAc and washed with 5% aq. solution of acetic acid. The organic phase was separated and aqueous layer was extracted twice with EtOAc, dried over magnesium sulfate, filtered and concentrated under reduced pressure to give crude product, which was purified by flash chromatography in 48% isolated yield. ¹H NMR (400 MHz, Methylene Chloride-d2) δ 9.72 (s, 1H), 8.92-8.77 (m, 1H), 8.71 (d, J=4.7 Hz, 1H), 7.85-7.71 (m, 2H), 7.71-7.55 (m, 3H), 7.45 (dd, J=8.0, 4.8 Hz, 1H), 4.83 (s, 2H), 3.28 (ddd, J=12.7, 9.0, 6.1 Hz, 1H), 3.13 (ddd, J=12.8, 9.1, 6.6 Hz, 1H), 1.85-1.59 (m, 2H), 1.59-1.38 (m, 2H), 0.97 (t, J=7.6 Hz, 3H). ESI-MS (m/z): 409.1 [M+H]⁺.

Enantiomers of EXAMPLE 19 were separated on a 1 cm Chiralpak AD-H column using 75% EtOH and 25% Hexanes with 5 mL/min flow rate, 400 μL injection (concentration 10 mg/ml) the 1st peak was at 5.5 min and the 2nd peak was at 14.7 min.

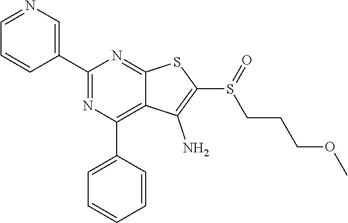 [SMILES: COCCCS(=O)c1sc2nc(-c3cccnc3)nc(-c3ccccc3)c2c1N]

EXAMPLE 20: 6-((3-methoxypropyl)sulfinyl)-4-phenyl-2-(pyridin-3-yl)thieno[2,3-d]pyrimidin-5-amine was prepared using synthetic procedures described for the preparation of the analog EXAMPLE 19. ¹H NMR (400 MHz, Methylene Chloride-d₂) δ 9.72 (dd, J=2.0, 1.0 Hz, 1H), 8.89-8.77 (m, 1H), 8.71 (dd, J=4.7, 1.7 Hz, 1H), 7.83-7.70 (m, 2H), 7.70-7.57 (m, 3H), 7.45 (ddd, J=8.0, 4.8, 1.0 Hz, 1H), 4.83 (s, 2H), 3.50 (t, J=5.9 Hz, 2H), 3.38-3.28 (m, 1H), 3.32 (s, 3H), 3.22 (ddd, J=12.8, 8.1, 6.4 Hz, 1H), 2.09-1.89 (m, 2H). ESI-MS (m/z): 425.1 [M+H]⁺.

Enantiomers of EXAMPLE 20 were separated on a 1 cm Chiralpak AD-H column using 100% EtOH with 5 mL/min flow rate, 500 μL injection (concentration 10 mg/ml) the 1st peak was at 8.34 min and the 2nd peak was at 27.5 min.

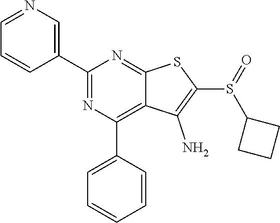 [SMILES: Nc1c(S(=O)C2CCC2)sc2nc(-c3cccnc3)nc(-c3ccccc3)c12]

EXAMPLE 21: 6-(cyclobutylsulfinyl)-4-phenyl-2-(pyridin-3-yl)thieno[2,3-d]pyrimidin-5-amine was prepared using synthetic procedures described for the preparation of the analog EXAMPLE 19. ¹H NMR (400 MHz, Methylene Chloride-d₂) δ 9.73 (s, 1H), 8.80 (d, J=8.0 Hz, 2H), 7.81-7.67 (m, 2H), 7.69-7.57 (m, 3H), 7.55-7.30 (m, 1H), 4.80 (s, 2H), 3.94 (p, J=8.1 Hz, 1H), 2.89-2.67 (m, 1H), 2.50-2.16 (m, 3H), 2.18-2.01 (m, 2H). ESI-MS (m/z): 407.1 [M+H]⁺.

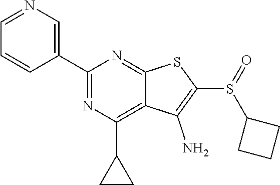 [SMILES: Nc1c(S(=O)C2CCC2)sc2nc(-c3cccnc3)nc(C3CC3)c12]

EXAMPLE 22: 6-(cyclobutylsulfinyl)-4-cyclopropyl-2-(pyridin-3-yl)thieno[2,3-d]pyrimidin-5-amine was prepared using synthetic procedures described for the preparation of the analog EXAMPLE 19. ¹H NMR (400 MHz, Methylene Chloride-d₂) δ 9.70-9.57 (m, 1H), 8.71 (dt, J=8.0, 2.0 Hz, 1H), 8.68 (dd, J=4.9, 1.7 Hz, 1H), 7.47-7.35 (m, 1H), 5.26 (s, 2H), 3.92 (p, J=8.0 Hz, 1H), 2.88-2.73 (m, 1H), 2.68 (tt, J=8.1, 4.7 Hz, 1H), 2.45-2.17 (m, 3H), 2.17-1.99 (m, 2H), 1.64-1.46 (m, 2H), 1.34-1.18 (m, 2H). ESI-MS (m/z): 371.1 [M+H]⁺.

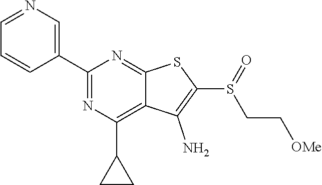 [SMILES: COCCS(=O)c1sc2nc(-c3cccnc3)nc(C3CC3)c2c1N]

EXAMPLE 23: 4-cyclopropyl-6-((2-methoxyethyl)sulfinyl)-2-(pyridin-3-yl)thieno[2,3-d]pyrimidin-5-amine was prepared using synthetic procedures described for the preparation of the analog EXAMPLE 19. ¹H NMR (400 MHz, Methylene Chloride-d₂) δ 9.63 (dd, J=2.2, 0.9 Hz, 1H), 8.71 (ddd, J=8.0, 2.2, 1.7 Hz, 1H), 8.68 (dd, J=4.8, 1.7 Hz, 1H), 7.42 (ddd, J=8.0, 4.8, 0.9 Hz, 1H), 5.25 (s, 2H), 3.86 (ddd, J=10.5, 7.6, 4.0 Hz, 1H), 3.68 (ddd, J=10.5, 6.3, 4.3 Hz, 1H), 3.58 (ddd, J=13.0, 6.3, 4.0 Hz, 1H), 3.39 (s, 3H), 3.27 (ddd, J=13.1, 7.5, 4.3 Hz, 1H), 2.66 (tt, J=8.0, 4.7 Hz, 1H), 1.59-1.54 (m, 2H), 1.30-1.25 (m, 2H). ESI-MS (m/z): 375.1 [M+H]⁺.

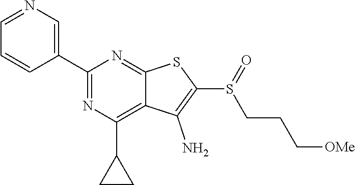 [SMILES: COCCCS(=O)c1sc2nc(-c3cccnc3)nc(C3CC3)c2c1N]

EXAMPLE 24: 4-cyclopropyl-6-((3-methoxypropyl)sulfinyl)-2-(pyridin-3-yl)thieno[2,3-d]pyrimidin-5-amine was prepared using synthetic procedures described for the preparation of the analog EXAMPLE 19. ¹H NMR (400 MHz, Methylene Chloride-d₂) δ 9.63 (dd, J=2.2, 0.9 Hz, 1H), 8.71 (ddd, J=8.0, 2.2, 1.7 Hz, 1H), 8.68 (dd, J=4.8, 1.7 Hz, 1H), 7.41 (ddd, J=8.0, 4.8, 0.9 Hz, 1H), 5.30 (s, 2H), 3.52 (t, J=5.9 Hz, 2H), 3.37-3.28 (m, 1H), 3.34 (s, 3H), 3.22 (ddd, J=13.0, 8.3, 6.4 Hz, 1H), 2.67 (tt, J=8.0, 4.7 Hz, 1H), 2.11-1.91 (m, 2H), 1.60-1.47 (m, 2H), 1.30-1.22 (m, 2H). ESI-MS (m/z): 389.1 [M+H]⁺.

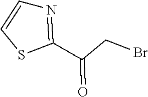

### Example 25A: 2-bromo-1-(thiazol-2-yl)ethan-1-one. n-BuLi (24.7 mL, 61.7

mmol, 2.5 M in Hexane) was added dropwise to a solution of 2-thiazole (5.0 g, 59 mmol) in anhydrous diethyl ether (50 mL) at −78° C. After 15 minutes, ethylbromoacetate (6.84 mL, 61.7 mmol) was added, the cold bath was removed and the solution was allowed to warm to room temperature. The reaction mixture was treated with AcOH (7 mL) and then diluted with water (100 mL) and ether (60 mL). The organic layer was separated, dried over Na2SO4, filtered and concentrated under reduced pressure. The crude product was suspended in hexanes and heated to reflux for 15 minutes then the product was decanted off leaving the impure oil. This was repeated 5 times to give a white solid with 88% yield. ¹H NMR (400 MHz, CDCl3) δ 8.05 (d, J=3.0 Hz, 1H), 7.77 (d, J=3.0 Hz, 1H), 4.71 (s, 2H).

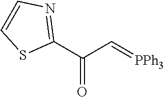

Example 25B: 1-(thiazol-2-yl)-2-(triphenyl-γ5-phosphanylidene)ethan-1-one. To a solution of 2-bromo-1-(thiazol-2-yl)ethan-1-one (10.7 g, 0.0517 mol) in toluene (337.7 mL), triphenylphosphine (14.1 g, 0.0539 mol) was added portion wise. The mixture was stirred at room temperature for 3 hours. The yellowish precipitate was removed by filtration, and was washed several times with toluene and then petroleum ether. Water was added to the precipitate and was treated dropwise with 1N NaOH to pH 10 (at pH 7 there was a color change from yellow to orange). The mixture was stirred for 30 minutes at room temperature. The precipitate was removed by filtration and washed several times with water. The resulting orange solid, was heated at 50° C. under vacuum to remove any water, to give dry product in 96% yield. ¹H NMR (400 MHz, CDCl3) δ 7.82 (d, J=3.1 Hz, 1H), 7.72 (ddd, J=12.8, 8.3, 1.4 Hz, 6H), 7.61-7.54 (m, 3H), 7.51-7.45 (m, 6H), 7.38 (dd, J=3.1, 1.3 Hz, 1H), 5.00 (d, J=23.3 Hz, 1H). ESI-MS (m/z): 387.9 [M+H]⁺.

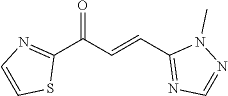

Example 25C: (E)-3-(1-methyl-1H-1,2,4-triazol-5-yl)-1-(thiazol-2-yl)prop-2-en-1-one To a solution of 1,5-dimethyl-1H-imidazole-2-carbaldehyde (2.25 mmol, 250 mg) in 5 ml of CH₃CN was added 1-(thiazol-2-yl)-2-(triphenyl-γ5-phosphanylidene)ethan-1-one (2.25 mmol, 871.6 mg, 1.0 equiv.). The reaction mixture was stirred at 90° C. for 48 h. Once complete, solvent was evaporated and residue was purified by flash chromatography to give 306 mg of product ¹H NMR (400 MHz, Chloroform-d) δ 8.39 (d, J=15.5 Hz, 1H), 8.09 (d, J=3.0 Hz, 1H), 7.97 (s, 1H), 7.82 (d, J=15.6 Hz, 1H), 7.75 (d, J=3.0 Hz, 1H), 4.05 (s, 3H). ESI-MS (m/z): 221.0 [M+H]⁺.

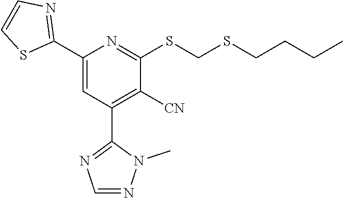 [SMILES: CCCCSCSc1nc(-c2nccs2)cc(-c2ncnn2C)c1C#N]

Example 25D: 2-(((butylthio)methyl)thio)-4-(1-methyl-1H-1,2,4-triazol-5-yl)-6-(thiazol-2-yl)nicotinonitrile. To a suspension of (E)-3-(1-methyl-1H-1,2,4-triazol-5-yl)-1-(thiazol-2-yl)prop-2-en-1-one (0.45 mmol, 100 mg) and 2-cyanothioacetamide (1.35 mmol, 135 mg, 3.0 equiv.) in EtOH (1.5 mL) 3 drops of piperidine were added. After being stirred at 80° C. for 2 h, EtOH was evaporated and crude product was redissolved in 2 mL of CH₃CN. Butyl(chloromethyl)sulfane (1.30 mmol, 180 mg, 3.0 equiv.) and Et₃N (1.30 mmol, 131 mg, 181 μL) were then added and the reaction mixture was stirred at 80° C. for 20 min. Once complete, the reaction was diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 119 mg of the mixture of desired product ESI-MS (m/z): 403.6 [M+H]⁺ and des CN byproduct: ESI-MS (m/z): 378.1 [M+H]⁺.

¹H NMR (400 MHz, Chloroform-d) δ 8.09 (s, 1H), 8.08 (s, 1H), 8.00 (d, J=3.1 Hz, 1H), 7.61 (d, J=3.1 Hz, 1H), 4.52 (s, 2H), 4.01 (s, 3H), 275 (t, J=7.2 Hz, 2H), 1.69-1.58 (m, 2H), 1.47-1.33 (m, 2H), 0.91 (t, J=7.3 Hz, 3H). ESI-MS (m/z): 403.6 [M+H]+

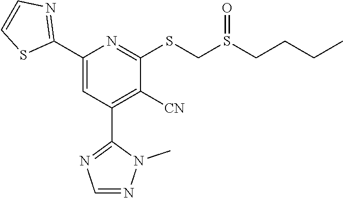 [SMILES: CCCCS(=O)CSc1nc(-c2nccs2)cc(-c2ncnn2C)c1C#N]

Example 25E: 2-(((butylsulfinyl)methyl)thio)-4-(1-methyl-1H-1,2,4-triazol-5-yl)-6-(thiazol-2-yl)nicotinonitrile. To the solution of the product from the previous step (119 mg) in CHCl₃/AcOH (1:1, 0.15 M) was added H₂O₂ (68 μL, 30% solution in water). The reaction mixture was stirred at 32° C. for 40 min. Once complete, the reaction was diluted with EtOAc and was washed with saturated NaHCO₃ solution, dried over magnesium sulfate, filtered and concentrated under reduce pressure to give 89 mg of desired compound and des CN desired compound. ¹H NMR (400 MHz, Chloroform-d) δ 8.17 (s, 1H), 8.07 (s, 1H), 8.00 (d, J=3.1 Hz, 1H), 7.64 (d, J=3.1 Hz, 1H), 4.69 (d, J=13.2 Hz, 1H), 4.45 (d, J=13.2 Hz, 1H), 4.03 (s, 3H), 3.02-2.88 (m, 1H), 2.88-2.77 (m, 1H), 1.88-1.77 (m, 2H), 1.54-1.41 (m, 2H), 0.95 (t, J=7.3 Hz, 3H). ESI-MS (m/z): 419.0 [M+H]⁺.

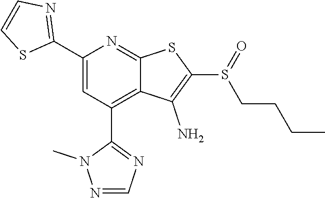 [SMILES: CCCCS(=O)c1sc2nc(-c3nccs3)cc(-c3ncnn3C)c2c1N]

EXAMPLE 25: 2-(butylsulfinyl)-4-(1-methyl-1H-1,2,4-triazol-5-yl)-6-(thiazol-2-yl)thieno[2,3-b]pyridin-3-amine. To the solution of 2-(((butylsulfinyl)methyl)thio)-4-(1-methyl-1H-1,2,4-triazol-5-yl)-6-(thiazol-2-yl)nicotinonitrile (43 mg) in DMF (700 μl)/MeOH (350 μl) was added KOH (0.06 mmol, 3.4 mg, 1.8 M in water). The reaction mixture was stirred at 32° C. for 10 min. Once complete, the reaction was diluted with EtOAc and acidified to pH 7 with 5% aq. solution of AcOH, the organic phase was separated and aqueous layer was extracted twice with EtOAc, dried over magnesium sulfate, filtered and concentrated under reduced pressure. The crude product was purified by flash chromatography to give desired product. ¹H NMR (400 MHz, Methylene Chloride-d₂) δ 8.26 (s, 1H), 8.09 (s, 1H), 7.97 (d, J=3.1 Hz, 1H), 7.60 (d, J=3.1 Hz, 1H), 5.67 (s, 2H), 4.03 (s, 3H), 3.26 (ddd, J=12.7, 9.0, 6.2 Hz, 1H), 3.12 (ddd, J=12.8, 9.0, 6.6 Hz, 1H), 1.86-1.67 (m, 2H), 1.57-1.39 (m, 2H), 0.95 (t, J=7.3 Hz, 3H). ESI-MS (m/z): 419.1 [M+H]⁺.

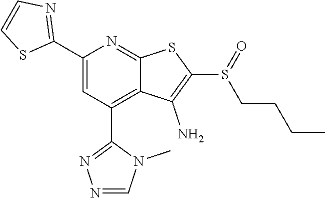 [SMILES: CCCCS(=O)c1sc2nc(-c3nccs3)cc(-c3nncn3C)c2c1N]

EXAMPLE 26: 2-(butylsulfinyl)-4-(4-methyl-4H-1,2,4-triazol-3-yl)-6-(thiazol-2-yl)thieno[2,3-b]pyridin-3-amine was prepared in 12% isolated yield via standard cyclization reaction with potassium hydroxide, using the synthetic procedures described for the preparation of the analog EXAMPLE 25. ¹H NMR (400 MHz, Methylene Chloride-d₂) δ 8.38 (s, 1H), 8.23 (s, 1H), 7.97 (d, J=3.2 Hz, ¹H), 7.60 (d, J=3.1 Hz, 1H), 5.63 (s, 2H), 3.80 (s, 3H), 3.26 (ddd, J=12.9, 9.1, 6.2 Hz, 1H), 3.12 (ddd, J=12.8, 9.1, 6.6 Hz, 1H), 1.89-1.66 (m, 2H), 1.57-1.42 (m, 2H), 0.95 (t, J=7.4 Hz, 3H). ESI-MS (m/z): 419.1 [M+H]⁺.

Enantiomers of EXAMPLE 26 were separated on a 2 cm Chiralcel OD-H column using 100% EtOH with 10 mL/min flow rate, 600 μL injection (concentration 10 mg/ml) the 1st peak was at 15.0 min and the 2nd peak was at 21.0 min.

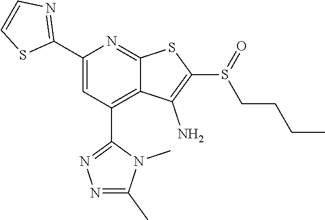 [SMILES: CCCCS(=O)c1sc2nc(-c3nccs3)cc(-c3nnc(C)n3C)c2c1N]

EXAMPLE 27: 2-(butylsulfinyl)-4-(4,5-dimethyl-4H-1,2,4-triazol-3-yl)-6-(thiazol-2-yl)thieno[2,3-b]pyridin-3-amine was prepared in 12% isolated yield via standard cyclization reaction with potassium hydroxide, using the synthetic procedures described for the preparation of the analog EXAMPLE 25. ¹H NMR (400 MHz, Methylene Chloride-d₂) δ 8.18 (s, 1H), 7.96 (d, J=3.1 Hz, 1H), 7.59 (d, J=3.2 Hz, 1H), 5.71 (s, 2H), 3.65 (s, 3H), 3.26 (ddd, J=12.8, 9.1, 6.1 Hz, 1H), 3.11 (ddd, J=12.8, 9.2, 6.5 Hz, 1H), 2.57 (s, 3H), 1.84-1.66 (m, 2H), 1.56-1.40 (m, 2H), 0.95 (t, J=7.3 Hz, 3H). ESI-MS (m/z): 433.1 [M+H]⁺.

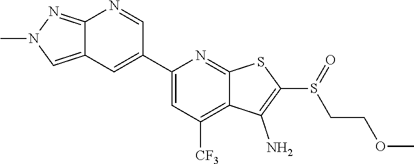

EXAMPLE 28: 2-((2-methoxyethyl)sulfinyl)-6-(2-methyl-2H-pyrazolo[3,4-b]pyridin-5-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.43 (d, J=2.4 Hz, 1H), 8.83 (d, J=2.4 Hz, 1H), 8.14 (s, 1H), 8.11 (s, 1H), 5.18 (s, 2H), 4.29 (s, 3H), 3.87 (ddd, J=9.7, 7.5, 3.5 Hz, 1H), 3.74-3.56 (m, 2H), 3.39 (s, 3H), 3.32 (ddd, J=13.0, 7.6, 3.7 Hz, 1H). ESI-MS (m/z): 456.2 [M+H]⁺.

Enantiomers of EXAMPLE 28 were separated on a 2 cm Chiralpak AD-H column using Hexanes 20% and EtOH 80% with 10 mL/min flow rate, 600 μL injection (concentration 10 mg/ml). The 1st peak was at 13.0 min and the 2nd peak was at 20.0 min.

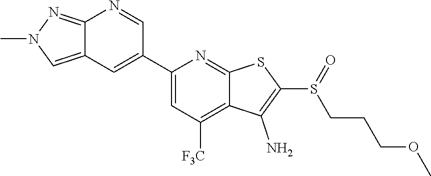

EXAMPLE 29: 2-((3-methoxypropyl)sulfinyl)-6-(2-methyl-2H-pyrazolo[3,4-b]pyridin-5-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.43 (d, J=2.3 Hz, 1H), 8.83 (d, J=2.3 Hz, 1H), 8.14 (s, 1H), 8.11 (s, 1H), 5.23 (s, 2H), 4.29 (s, 3H), 3.53 (t, J=5.9 Hz, 2H), 3.43-3.35 (m, 1H), 3.34 (s, 3H), 3.26 (ddd, J=13.0, 8.3, 6.5 Hz, 1H), 2.11-1.96 (m, 2H). ESI-MS (m/z): 470.2 [M+H]⁺.

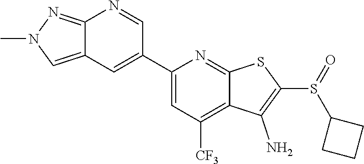 [SMILES: Cn1cc2cc(-c3cc(C(F)(F)F)c4c(N)c(S(=O)C5CCC5)sc4n3)cnc2n1]

EXAMPLE 30: 2-(cyclobutylsulfinyl)-6-(2-methyl-2H-pyrazolo[3,4-b]pyridin-5-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.42 (d, J=2.3 Hz, 1H), 8.81 (d, J=2.3 Hz, 1H), 8.12 (s, 1H), 8.11 (s, 1H), 5.17 (s, 2H), 4.29 (s, 3H), 4.05-3.91 (m, 1H), 2.89-2.72 (m, 1H), 2.47-2.36 (m, 1H), 2.36-2.20 (m, 2H), 2.16-2.03 (m, 2H). ESI-MS (m/z): 452.1 [M+H]⁺.

Enantiomers of EXAMPLE 30 were separated on a 2 cm Chiralcel OD-H column using 40% Hexanes and 60% EtOH with 10 mL/min flow rate, 600 μL injection (concentration 10 mg/ml) the 1st peak was at 17.5 min and the 2nd peak was at 23.0 min.

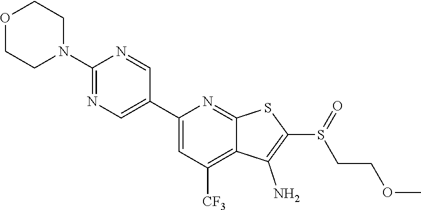 [SMILES: COCCS(=O)c1sc2nc(-c3cnc(N4CCOCC4)nc3)cc(C(F)(F)F)c2c1N]

EXAMPLE 31: 2-((2-methoxyethyl)sulfinyl)-6-(2-morpholinopyrimidin-5-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.08 (s, 2H), 7.92 (s, 1H), 5.15 (s, 2H), 3.97-3.90 (m, 4H), 3.86 (ddd, J=9.8, 7.6, 3.7 Hz, 1H), 3.81-3.72 (m, 4H), 3.71-3.57 (m, 2H), 3.38 (s, 3H), 3.29 (ddd, J=12.9, 7.6, 3.9 Hz, 1H). ESI-MS (m/z): 488.2 [M+H]⁺.

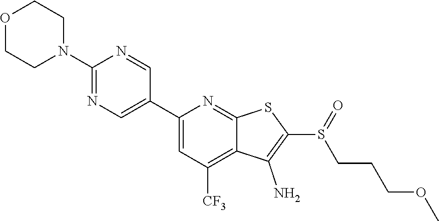 [SMILES: COCCCS(=O)c1sc2nc(-c3cnc(N4CCOCC4)nc3)cc(C(F)(F)F)c2c1N]

EXAMPLE 32: 2-((3-methoxypropyl)sulfinyl)-6-(2-morpholinopyrimidin-5-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.08 (s, 2H), 7.92 (s, 1H), 5.19 (s, 2H), 4.00-3.89 (m, 4H), 3.82-3.71 (m, 4H), 3.51 (t, J=5.9 Hz, 2H), 3.41-3.29 (m, 1H), 3.33 (s, 3H), 3.24 (ddd, J=13.0, 8.2, 6.5 Hz, 1H), 2.10-1.95 (m, 2H). ESI-MS (m/z): 502.1 [M+H]⁺.

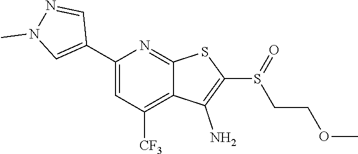

EXAMPLE 33: 2-((2-methoxyethyl)sulfinyl)-6-(1-methyl-1H-pyrazol-4-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 8.09 (s, 1H), 8.05 (d, J=0.8 Hz, 1H), 7.74 (d, J=0.8 Hz, 1H), 5.11 (s, 2H), 3.97 (s, 3H), 3.90-3.80 (m, 1H), 3.71-3.54 (m, 2H), 3.38 (s, 3H), 3.35-3.22 (m, 1H). ESI-MS (m/z): 405.1 [M+H]⁺.

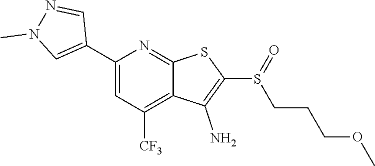

EXAMPLE 34: 2-((3-methoxypropyl)sulfinyl)-6-(1-methyl-1H-pyrazol-4-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 8.08 (s, 1H), 8.05 (d, J=0.8 Hz, 1H), 7.74 (d, J=0.8 Hz, 1H), 5.15 (s, 2H), 3.97 (s, 3H), 3.51 (t, J=5.9 Hz, 2H), 3.41-3.29 (m, 1H), 3.33 (s, 3H), 3.22 (ddd, J=13.0, 8.1, 6.7 Hz, 1H), 2.11-1.93 (m, 2H). ESI-MS (m/z): 419.1 [M+H]⁺.

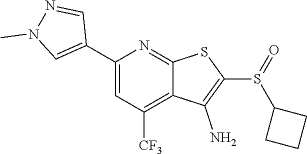 [SMILES: Cn1cc(-c2cc(C(F)(F)F)c3c(N)c(S(=O)C4CCC4)sc3n2)cn1]

EXAMPLE 35: 2-(cyclobutylsulfinyl)-6-(1-methyl-1H-pyrazol-4-yl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 8.07 (s, 1H), 8.04 (s, 1H), 7.73 (s, 1H), 5.10 (s, 2H), 4.06-3.88 (m, 1H), 3.97 (s, 3H), 2.89-2.67 (m, 1H), 2.47-2.15 (m, 3H), 2.15-1.94 (m, 2H). ESI-MS (m/z): 401.1 [M+H]⁺.

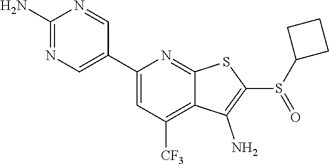 [SMILES: Nc1ncc(-c2cc(C(F)(F)F)c3c(N)c(S(=O)C4CCC4)sc3n2)cn1]

EXAMPLE 36: 6-(2-aminopyrimidin-5-yl)-2-(cyclobutylsulfinyl)-4-(trifluoromethyl)thieno[2,3-b]pyridin-3-amine was prepared using the synthetic procedures described for the preparation of EXAMPLE 7. ¹H NMR (400 MHz, Methylene Chloride-d₂) δ 9.05 (s, 2H), 7.92 (s, 1H), 5.40 (s, 2H), 5.15 (s, 2H), 3.97 (p, J=8.0 Hz, 1H), 2.87-2.68 (m, 1H), 2.51-2.35 (m, 1H), 2.35-2.14 (m, 2H), 2.14-1.92 (m, 2H). ESI-MS (m/z): 414.1 [M+H]⁺.

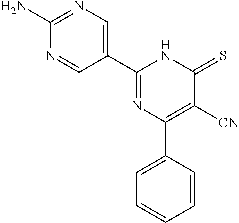 [SMILES: N#Cc1c(-c2ccccc2)nc(-c2cnc(N)nc2)[nH]c1=S]

EXAMPLE 37A: 2-(4-aminophenyl)-4-phenyl-6-thioxo-1,6-dihydropyrimidine-5-carbonitrile. A mixture of NaOiPr (1.75 mmol, 1.0 equiv, prepared in situ from sodium and dry iPrOH), 2-aminopyrimidine-5-carbothioamide (1.75 mmol, 270 mg, 1.0 equiv) and 2-(methoxy(phenyl)methylene)malononitrile (1.75 mmol, 322 mg, 1.0 equiv) in iPrOH (30 mL) was stirred for overnight at r.t. The reaction was acidified with con. HCl and then evaporated to give crude product as a solid (380 mg). ESI-MS (m/z): 307.1 [M+H].

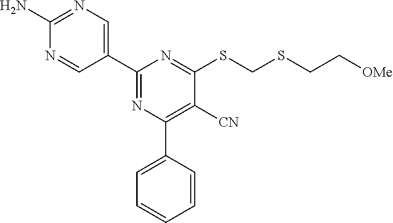 [SMILES: COCCSCSc1nc(-c2cnc(N)nc2)nc(-c2ccccc2)c1C#N]

EXAMPLE 37B: 2-(4-aminophenyl)-6-((((2-methoxyethyl)thio)methyl)thio)-4-phenyl-1,6-dihydropyrimidine-5-carbonitrile. A mixture of 2-(4-aminophenyl)-6-mercapto-4-phenyl-1,6-dihydropyrimidine-5-carbonitrile (0.33 mmol, 100 mg), (chloromethyl)(2-methoxyethyl)sulfane (0.36 mmol, 50 mg, 1.1 equiv) and Et₃N (0.98 mmol, 140 μl, 3.0 equiv) was stirred in dry CH₃CN (3 mL) for 20 min at room temperature. The reaction mixture was then diluted with EtOAc and water. The organic phase was separated and aqueous layer, dried over magnesium sulfate, filtered and concentrated under reduced pressure. The residue was purified by flash chromatography to give 37 mg of desired product. ¹H NMR (400 MHz, Chloroform-d) δ 9.42 (s, 2H), 8.25-7.94 (m, 2H), 7.81-7.48 (m, 3H), 5.72 (s, 2H), 4.61 (s, 2H), 3.69 (t, J=6.0 Hz, 2H), 3.39 (s, 3H), 2.92 (t, J=6.0 Hz, 2H). ESI-MS (m/z): 411.0 [M+H]⁺.

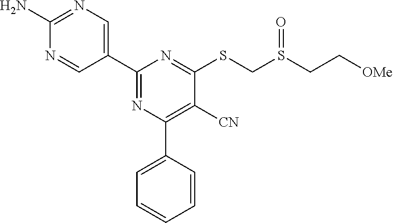 [SMILES: COCCS(=O)CSc1nc(-c2cnc(N)nc2)nc(-c2ccccc2)c1C#N]

EXAMPLE 37C: 2-(4-aminophenyl)-6-((((2-methoxyethyl)sulfinyl)methyl)thio)-4-phenyl-1,6-dihydropyrimidine-5-carbonitrile To the solution of 2-(4-aminophenyl)-6-((((2-methoxyethyl)thio)methyl)thio)-4-phenyl-1,6-dihydropyrimidine-5-carbonitrile (30 mg, 0.073 mmol) in CHCl₃ (135 μl) and AcOH (135 μl) was added H₂O₂ (17 μL, 30% solution in water). The reaction mixture was stirred at 32° C. for 40 min. Once complete, the reaction was diluted with EtOAc and was washed with saturated NaHCO₃ solution, dried over magnesium sulfate, filtered and concentrated under reduce pressure to give 18 mg of desired compound. ESI-MS (m/z): 427.0 [M+H]⁺.

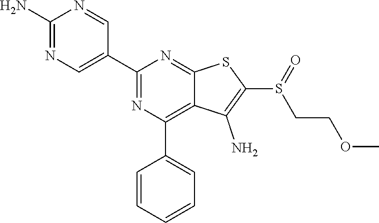 [SMILES: COCCS(=O)c1sc2nc(-c3cnc(N)nc3)nc(-c3ccccc3)c2c1N]

EXAMPLE 37: 2-(2-aminopyrimidin-5-yl)-6-((2-methoxyethyl)sulfinyl)-4-phenylthieno[2,3-d]pyrimidin-5-amine was prepared using the KOH cyclization procedure described for the preparation of the analog EXAMPLE 19. ¹H NMR (400 MHz, Methylene Chloride-d₂) δ 9.37 (s, 2H), 7.81-7.68 (m, 2H), 7.68-7.56 (m, 3H), 5.46 (s, 2H), 4.75 (s, 2H), 3.83 (ddd, J=11.0, 7.6, 4.0 Hz, 1H), 3.66 (ddd, J=10.6, 6.3, 4.3 Hz, 1H), 3.56 (ddd, J=13.1, 6.2, 4.0 Hz, 1H), 3.36 (s, 3H), 3.26 (ddd, J=13.2, 7.6, 4.3 Hz, 1H). ESI-MS (m/z): 427.1 [M+H]⁺.

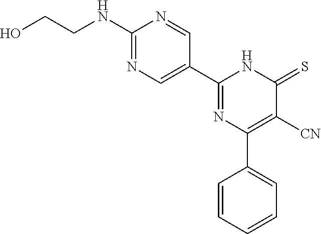 [SMILES: N#Cc1c(-c2ccccc2)nc(-c2cnc(NCCO)nc2)[nH]c1=S]

EXAMPLE 38A: 2′-((2-hydroxyethyl)amino)-4-phenyl-6-thioxo-1,6-dihydro-′-bipyrimidine]-5-carbonitrile was prepared from 2-((2-hydroxyethyl)amino)pyrimidine-5-carbothioamide and 2-(methoxy(phenyl)methylene)malononitrile, using the procedure described for the preparation of the analog EXAMPLE 37. ESI-MS (m/z): 351.1 [M+H]⁺.

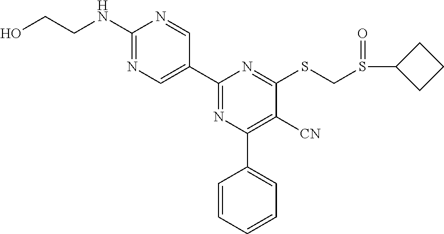 [SMILES: N#Cc1c(SCS(=O)C2CCC2)nc(-c2cnc(NCCO)nc2)nc1-c1ccccc1]

EXAMPLE 38B: 4-(((cyclobutylsulfinyl)methyl)thio)-2′-((2-hydroxyethyl)amino)-6-phenyl-[2,5′-bipyrimidine]-5-carbonitrile. A mixture of 2′-((2-hydroxyethyl)amino)-4-phenyl-6-thioxo-1,6-dihydro-[2,5′-bipyrimidine]-5-carbonitrile (0.11 mmol, 40 mg), ((chloromethyl)sulfinyl)cyclobutane (0.42 mmol, 21 mg, 1.2 equiv) and Et₃N (0.98 mmol, 46 μl, 3.0 equiv) was stirred in dry CH₃CN (3 mL) for 2 h at 80° C. The reaction mixture was then diluted with EtOAc and water. The organic phase was separated and aqueous layer was extracted twice with EtOAc. The combined extractions were washed with saturated NaCl solution, dried over magnesium sulfate, filtered and concentrated under reduced pressure. The residue was purified by flash chromatography to give 26 mg of desired product. ¹H NMR (400 MHz, Chloroform-d) δ 12.04 (s, 1H), 9.44 (s, 2H), 8.21-7.92 (m, 2H), 7.80-7.42 (m, 3H), 4.66 (d, J=13.3 Hz, 1H), 4.06 (d, J=13.0 Hz, 1H), 3.94-3.82 (m, 2H), 3.82-3.66 (m, 3H), 2.90-2.70 (m, 1H), 2.57-2.38 (m, 1H), 2.33-2.02 (m, 4H). ESI-MS (m/z): 467.1 [M+H]⁺.

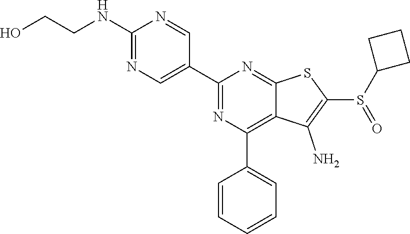 [SMILES: Nc1c(S(=O)C2CCC2)sc2nc(-c3cnc(NCCO)nc3)nc(-c3ccccc3)c12]

EXAMPLE 38: 2-((5-(5-amino-6-(cyclobutylsulfinyl)-4-phenylthieno[2,3-d]pyrimidin-2-yl)pyrimidin-2-yl)amino)ethan-1-ol was prepared using the KOH cyclization procedure described for the preparation of the analog EXAMPLE 19. ¹H NMR (400 MHz, Methylene Chloride-d₂) δ 9.31 (s, 2H), 7.76-7.66 (m, 2H), 7.66-7.56 (m, 3H), 5.98 (t, J=6.1 Hz, 1H), 4.74 (s, 2H), 4.00-3.78 (m, 3H), 3.78-3.56 (m, 2H), 2.86-2.64 (m, 1H), 2.44-1.89 (m, 5H)). ESI-MS (m/z): 467.1 [M+H]⁺.

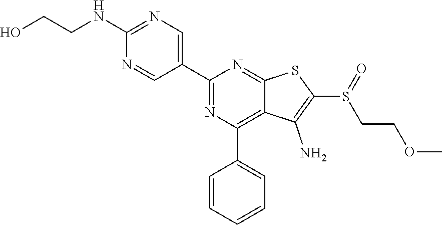 [SMILES: COCCS(=O)c1sc2nc(-c3cnc(NCCO)nc3)nc(-c3ccccc3)c2c1N]

EXAMPLE 39: 2-((5-(5-amino-6-((2-methoxyethyl)sulfinyl)-4-phenylthieno[2,3-d]pyrimidin-2-yl)pyrimidin-2-yl)amino)ethan-1-ol was prepared using the synthetic procedures described for the preparation of EXAMPLE 38. ¹H NMR (400 MHz, Methylene Chloride-d₂) δ 9.32 (s, 2H), 7.75-7.68 (m, 2H), 7.66-7.58 (m, 3H), 6.06 (t, J=6.3 Hz, 1H), 4.74 (s, 2H), 3.91-3.77 (m, 3H), 3.73-3.59 (m, 3H), 3.54 (ddd, J=13.0, 6.1, 4.0 Hz, 1H), 3.36 (s, 3H), 3.24 (ddd, J=13.0, 7.7, 4.3 Hz, 1H). ESI-MS (m/z): 471.1 [M+H]⁺.

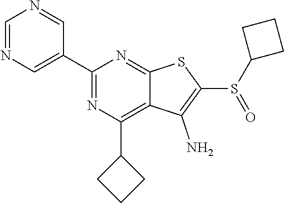 [SMILES: Nc1c(S(=O)C2CCC2)sc2nc(-c3cncnc3)nc(C3CCC3)c12]

EXAMPLE 40: 4-cyclobutyl-6-(cyclobutylsulfinyl)-2-(pyrimidin-5-yl)thieno[2,3-d]pyrimidin-5-amine was prepared using synthetic procedures described for the preparation of EXAMPLE 19. ¹H NMR (400 MHz, Methylene Chloride-d₂) δ 9.82 (s, 2H), 9.29 (s, 1H), 5.17 (s, 2H), 4.25 (p, J=8.2 Hz, 1H), 3.92 (p, J=8.1 Hz, 1H), 2.91-2.71 (m, 2H), 2.57-2.41 (m, 2H), 2.42-1.91 (m, 8H). ESI-MS (m/z): 386.1 [M+H]⁺.

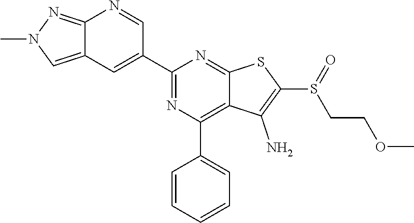

EXAMPLE 41: 6-((2-methoxyethyl)sulfinyl)-2-(2-methyl-2H-pyrazolo[3,4-b]pyridin-5-yl)-4-phenylthieno[2,3-d]pyrimidin-5-amine was prepared using synthetic procedures described for the preparation of EXAMPLE 38. ¹H NMR (400 MHz, Methylene Chloride-d₂) δ 9.82 (d, J=2.2 Hz, 1H), 9.27 (d, J=2.2 Hz, 1H), 8.09 (s, 1H), 7.80-7.74 (m, 2H), 7.68-7.61 (m, 3H), 4.78 (s, 2H), 4.27 (s, 3H), 3.84 (ddd, J=10.5, 7.6, 4.0 Hz, 1H), 3.67 (ddd, J=10.5, 6.3, 4.3 Hz, 1H), 3.58 (ddd, J=13.1, 6.3, 4.0 Hz, 1H), 3.37 (s, 3H), 3.28 (ddd, J=13.0, 7.6, 4.3 Hz, 1H). ESI-MS (m/z): 465.1 [M+H]⁺.

Enantiomers of EXAMPLE 41 were separated on a 2 cm Chiralpak AD-H column using Hexanes: EtOH (1:1) with 10 m/min flow rate, 600 μL injection (concentration 8 mg/ml) the 1st peak was at 22.0 min and the 2nd peak was at 35.0 min.

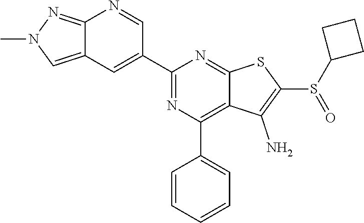 [SMILES: Cn1cc2cc(-c3nc(-c4ccccc4)c4c(N)c(S(=O)C5CCC5)sc4n3)cnc2n1]

EXAMPLE 42: 6-(cyclobutylsulfinyl)-2-(2-methyl-2H-pyrazolo[3,4-b]pyridin-5-yl)-4-phenylthieno[2,3-d]pyrimidin-5-amine was prepared using synthetic procedures described for the preparation of the analog EXAMPLE 19. ¹H NMR (400 MHz, Methylene Chloride-d₂) δ 9.82 (s, 1H), 9.26 (s, 1H), 8.09 (s, 1H), 7.83-7.71 (m, 2H), 7.71-7.57 (m, 3H), 4.78 (s, 2H), 4.27 (s, 3H), 4.03-3.88 (m, 1H), 2.83-2.70 (m, 1H), 2.47-1.92 (m, 5H). ESI-MS (m/z): 461.1 [M+H]⁺.

### General Procedure A

### Step 1: Aldol Reaction

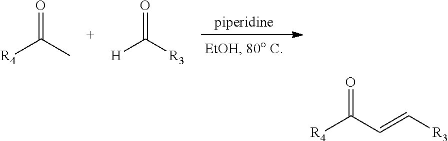

To a solution of the ketone (1.0 equiv) and the aldehyde (2.0 equiv) in ethanol was added piperidine (3.0 equiv). The mixture was stirred at 80° C. for 2 hours. After cooling, the mixture was concentrated and the residue was triturated with a 4:1 mixture of petroleum ether: ethyl acetate. The solid was filtered to give the enone.

### Step 2: Condensation with 2-Cyanothioacetamide and Oxidation

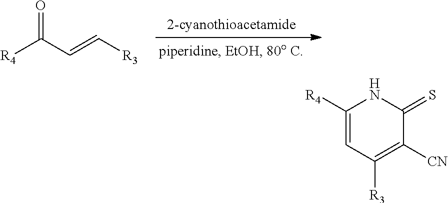

To a solution of the enone (1.0 equiv) in ethanol was added 2-cyanothioacetamide (3.0 equiv) and piperidine (2.0 equiv). The reaction mixture was stirred at 80° C. for 1 hour under an oxygen atmosphere (15 psi). The mixture was concentrated to give the crude pyridinethione.

### Step 3: Preparation of α-chlorosulfides

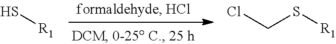

Hydrogen chloride gas was bubbled into a solution of the appropriate thiol (1.0 equiv) and formaldehyde (1.0 equiv) in dichloromethane. The mixture was stirred at 0° C. for 1 hour. The mixture was warmed to 25° C. and stirred for 24 hours. The mixture was concentrated to give the target compound.

### Step 4: Alkylation

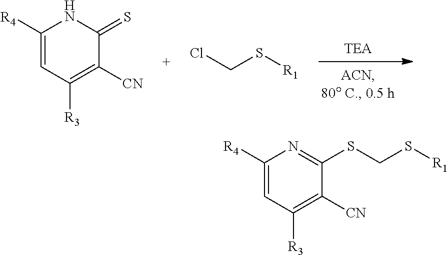

To a solution of the pyridinethione (1.0 equiv) in acetonitrile were added triethylamine (3.0 equiv) and the appropriate chloromethylsulfide (2.0 equiv). The mixture was stirred at 80° C. for 0.5 hour. The mixture was concentrated and the residue was purified by reversed-phase HPLC to give the target compound.

### Step 5: Oxidation to Sulfoxide

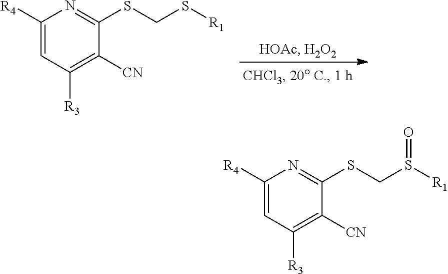

To a solution of the pyridinethiomethylsulfane (1 equiv) in chloroform was added acetic acid (25 equiv) and hydrogen peroxide (30% purity, 1.5 equiv). The mixture was stirred at 20° C. for 1 hour. The mixture was neutralized with saturated sodium bicarbonate solution and extracted with dichloromethane. The combined organic phases were concentrated to give the target sulfoxide.

### Step 6: Cyclization

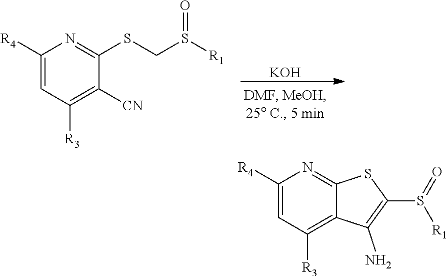

To a solution of the sulfoxide (1 equiv) in methanol and N, N-dimethylformamide was added potassium hydroxide solution (5%, 0.6 equiv). The mixture was stirred at 20° C. for 5 minutes. The mixture was neutralized with aqueous acetic acid (10%) and concentrated. The residue was purified by reversed-phase HPLC to give the final target compound.

The following compounds were prepared from known starting materials using General Procedure A. For select compounds, the enantiomers were resolved from the racemate using chiral supercritical fluid chromatography (SFC) and are reported with enantiomeric excess (ee) and/or optical rotation values. In some cases, only the more active R-(+) isomer was prepared/isolated.

| Ex. | | | LCMS | Chiral |
|---|---|---|---|---|
| | | 400 MHz NMR (δ; CDCl₃ | (ES⁺; | compound |
| No. | Structure | unless otherwise noted) | MH⁺) | info |
| 43 | 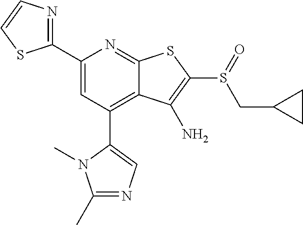 [SMILES: Cc1ncc(-c2cc(-c3nccs3)nc3sc(S(=O)CC4CC4)c(N)c23)n1C] | 8.10 (s, 1H), 7.97 (d, J = 3.2 Hz, 1H), 7.55 (d, J = 3.2 Hz, 1H), 7.16 (s, 1H), 4.82 (s, 2H), 3.44 (s, 3H), 3.14-3.26 (m, 2H), 2.53 (s, 3H), 1.06-1.08 (m, 1H), 0.71-0.74 (m, 2H), 0.36-0.46 (m, 2H). | 430.1 | |
| 44 | 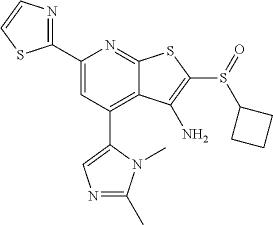 [SMILES: Cc1ncc(-c2cc(-c3nccs3)nc3sc(S(=O)C4CCC4)c(N)c23)n1C] | DMSO-d6 8.06 (s, 1H), 7.95 (d, J = 3.2 Hz, 1H), 7.53 (d, J = 3.2 Hz, 1H), 7.16 (s, 1H), 4.75 (s, 2H), 3.91-3.99 (m, 1H), 3.41 (s, 3H), 2.50-2.83 (m, 1H), 2.40 (s, 3H), 2.07-2.38 (m, 5 H). | 430.1 | R-isomer: [α] = +143.2° S-isomer: [α] = −147.3° |
| 45 | 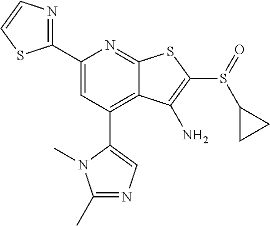 [SMILES: Cc1ncc(-c2cc(-c3nccs3)nc3sc(S(=O)C4CC4)c(N)c23)n1C] | 8.07 (s, 1H), 7.95 (d, J = 3.2 Hz, 1H), 7.53 (d, J = 3.2 Hz, 1H), 7.14 (s, 1H), 4.76 (s, 2H), 3.43 (s, 3H), 2.66 (s, 1H), 2.51 (s, 3H), 1.41- 1.43 (m, 1H), 1.22-1.12 (m, 3H) | 416.1 | |
| 46 | 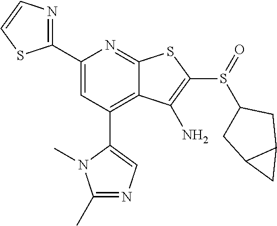 [SMILES: Cc1ncc(-c2cc(-c3nccs3)nc3sc(S(=O)C4CC5CC5C4)c(N)c23)n1C] | 8.07 (s, 1H), 7.96 (d, J = 3.2 Hz, 1H), 7.54 (d, J = 3.2 Hz, 1H), 7.14 (s, 1H), 4.72 (s, 2H), 4.03 (s, 1H), 3.42 (s, 3H), 2.53- 2.60 (m, 1H), 2.51 (s, 3H), 2.20-2.45 (m, 2H), 1.73-1.83 (m, 1H), 1.48-1.49 (m, 2H), 1.38-1.39 (m, 1H), 0.61-0.65 (m, 1H), 0.38-0.42 (m, 1H) | 456.1 | |
| 47 | 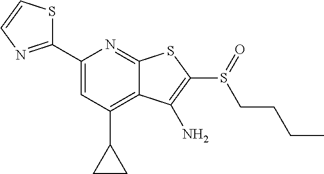 [SMILES: CCCCS(=O)c1sc2nc(-c3nccs3)cc(C3CC3)c2c1N] | 7.96-7.95 (m, 2H), 7.50 (d, J = 3.2 Hz, 1H), 5.61 (s, 2H), 3.38- 3.31 (m, 1H), 3.22-3.15 (m, 1H), 2.58-2.51 (m, 1H), 1.87-1.73 (m, 2H), 1.59-1.48 (m, 2H), 1.23- 1.19 (m, 2H), 1.19-1.07 (m, 2H), 0.99 (d, J = 7.2 Hz, 3H) | 378.2 | |
| 48 | 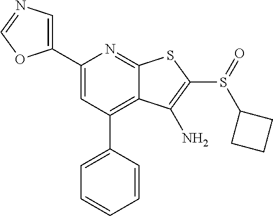 [SMILES: Nc1c(S(=O)C2CCC2)sc2nc(-c3cnco3)cc(-c3ccccc3)c12] | 8.01 (s, 1H), 7.82 (s, 1H), 7.58- 7.54 (m, 3H), 7.50 (s, 1H), 7.50- 7.45 (m, 2H), 4.59 (s, 2H), 4.01- 3.93 (m, 1H), 2.85-2.74 (m, 1H), 2.44-2.31 (m, 2H), 2.30-2.18 (m, 1H), 2.14-2.02 (m, 2H). | 396.1 | |
| 49 | 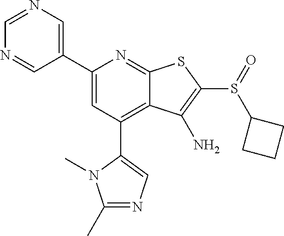 [SMILES: Cc1ncc(-c2cc(-c3cncnc3)nc3sc(S(=O)C4CCC4)c(N)c23)n1C] | 9.42 (s, 2H), 9.31 (s, 1H), 7.59 (s, 1H), 7.15 (s, 1H), 4.73 (s, 2H), 3.91-3.99 (m, 1H), 3.40 (s, 3H), 2.84 (s, 1H), 2.52 (s, 3H), 2.27-2.48 (m, 3H), 2.03-2.15 (m, 2H) | 425.1 | |
| 50 | 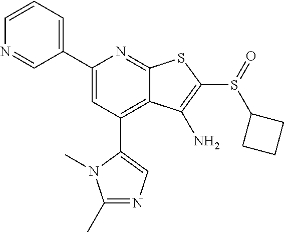 [SMILES: Cc1ncc(-c2cc(-c3cccnc3)nc3sc(S(=O)C4CCC4)c(N)c23)n1C] | 9.28 (d, J = 2.4 Hz, 1H), 8.71 (dd, J₁ = 1.6, J₂ = 4.8 Hz, 1H), 8.42- 8.45 (m, 1H), 7.60 (s, 1H), 7.45 (dd, J₁ = 3.2, J₂ = 4.8 Hz, 1H), 7.14 (s, 1H), 4.70 (s, 2H), 3.91-3.99 (m, 1H), 3.40 (s, 3H), 2.52 (s, 1H), 2.52 (s, 3H), 2.26-2.46 (m, 3H), 2.05-2.17 (m, 2H). | 424.1 | |
| 51 | 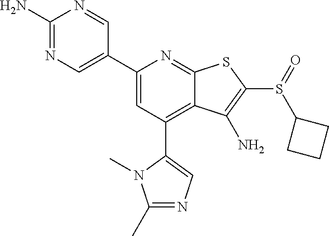 [SMILES: Cc1ncc(-c2cc(-c3cnc(N)nc3)nc3sc(S(=O)C4CCC4)c(N)c23)n1C] | 9.02 (s, 2H), 7.43 (s, 1H), 7.12 (s, 1H), 5.31 (s, 2H), 4.65 (s, 2H), 3.98-3.90 (m, 1H), 3.38 (s, 3H), 2.82-2.83 (m, 1H), 2.51 (s, 3H), 2.40-2.35 (m, 2H), 2.12-2.11 (m, 1H), 2.09-2.06 (m, 2H) | 440.2 | |
| 52 | 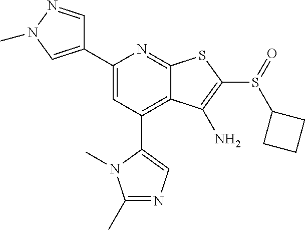 [SMILES: Cc1ncc(-c2cc(-c3cnn(C)c3)nc3sc(S(=O)C4CCC4)c(N)c23)n1C] | DMSO-d6 8.46 (m, 1H), 8.14 (s, 1H), 7.60 (s, 1H), 7.03 (s, 1H), 5.12 (s, 2H), 3.88 (s, 3H), 3.78-3.84 (m, 1H), 3.36 (s, 3H), 2.61-2.64 (m, 1H), 2.40 (s, 3H), 2.13-2.18 (m, 3H), 1.90-2.01 (m, 2H). | 427.2 | |
| 53 | 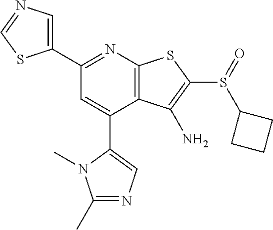 [SMILES: Cc1ncc(-c2cc(-c3cncs3)nc3sc(S(=O)C4CCC4)c(N)c23)n1C] | 8.90 (s, 1H), 8.42 (s, 1H), 7.50 (s, 1H), 7.13 (s, 1H), 4.66 (s, 2H), 3.91-3.95 (m, 1H), 3.39 (s, 3H), 2.82 (s, 1H), 2.51 (s, 3H), 2.36-2.43 (m, 2H), 2.26-2.28 (m, 1H), 2.03- 2.14 (m, 2H) | 430.1 | |
| 54 | 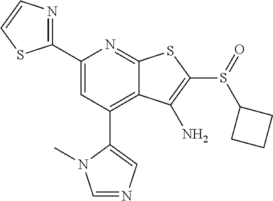 [SMILES: Cn1cncc1-c1cc(-c2nccs2)nc2sc(S(=O)C3CCC3)c(N)c12] | 8.1 (s, 1H), 7.95 (d, J = 2.8 Hz, 1H), 7.68 (s, 1H), 7.53 (d, J = 3.2 Hz, 1H), 4.66 (s, 2H), 3.91-3.99 (m, 1H), 3.54 (s, 3H), 2.83-2.88 (m, 1H), 2.08-2.42 (m, 5H). | 416.2 | |
| 55 | 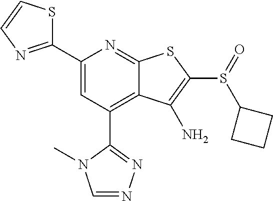 [SMILES: Cn1cnnc1-c1cc(-c2nccs2)nc2sc(S(=O)C3CCC3)c(N)c12] | 8.37 (s, 1H), 8.19 (s, 1H), 7.96 (d, J = 3.2 Hz, 1H), 7.56 (d, J = 3.2 Hz, 1H), 5.58 (s, 2H), 3.97-3.93 (m, 1H), 3.83 (s, 3H), 2.87-2.81 (m, 1H), 2.41-2.38 (m, 1H), 2.34- 2.25 (m, 2H), 2.11-2.06 (m, 2H) | 417.1 | |
| 56 | 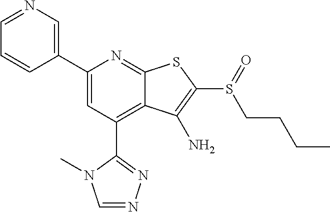 [SMILES: CCCCS(=O)c1sc2nc(-c3cccnc3)cc(-c3nncn3C)c2c1N] | DMSO-d6 9.42 (d, J = 1.6 Hz, 1H), 8.91 (s, 1H), 8.71 (d, J = 4.8 Hz, 1H), 8.62 (d, J = 8.0 Hz, 1H), 8.34 (s, 1H), 7.60 (dd, J = 4.8, 7.6 Hz, 1H), 6.10 (s, 2H), 3.82 (s, 3H), 3.12-3.19 (m, 1H), 3.00-3.07 (m, 1H), 1.54- 1.65 (m, 2H), 1.39-1.48 (m, 2H), 0.89 (t, J = 7.2 Hz, 3H) | 413.2 | |
| 57 | 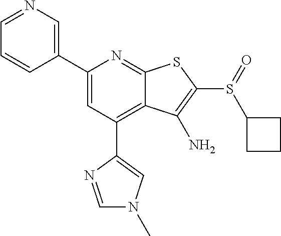 [SMILES: Cn1cnc(-c2cc(-c3cccnc3)nc3sc(S(=O)C4CCC4)c(N)c23)c1] | 9.18 (d, J = 1.6 Hz, 1H), 8.61 (d, J₁ = 1.6 Hz, J₁ = 4.8 Hz,, 1H), 8.38- 8.41 (m, J = 1.94, 8.13 Hz, 1H), 7.66 (s, 1H), 7.56 (s, 1H), 7.35-7.39 (m, 2H), 6.84 (s, 2H), 3.87-3.95 (m, 1H), 3.78 (s, 3H), 2.71-2.81 (m, 1H), 2.25-2.35 (m, 2H), 2.11-2.17 (m, 1H), 1.96-2.04 (m, 2H). | 448.1 | |
| 58 | 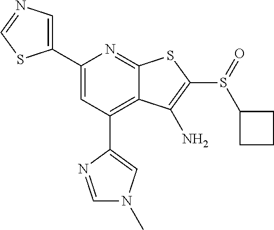 [SMILES: Cn1cnc(-c2cc(-c3cncs3)nc3sc(S(=O)C4CCC4)c(N)c23)c1] | 8.87 (s, 1H), 8.41 (s, 1H), 7.63 (s, 2H), 7.46 (s, 1H), 6.87 (s, 2H), 3.92-4.00 (m, 1H), 3.85 (s, 3H), 2.77-2.86 (m, 1H), 2.33-2.40 (m, 2H), 2.17-2.23 (m, 1H), 2.02-2.11 (m, 2H) | 416.2 | |
| 59 | 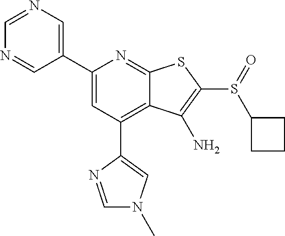 [SMILES: Cn1cnc(-c2cc(-c3cncnc3)nc3sc(S(=O)C4CCC4)c(N)c23)c1] | 9.34 (s, 2H), 9.21 (s, 1H), 7.63 (s, 1H), 7.56 (s, 1H), 7.40 (s, 1H), 6.86 (s, 2H), 3.87-3.95 (m, 1H), 3.79 (s, 3H), 2.73-2.79 (m, 1H), 2.28-2.31 (m, 2H), 1.98-2.02 (m, 3H) | 411.1 | R-isomer: 98.1% ee; [α] = +114.3° S-isomer: 100% ee; [α] = −17.7° |
| 60 | 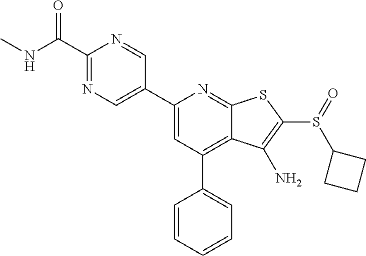 [SMILES: CNC(=O)c1ncc(-c2cc(-c3ccccc3)c3c(N)c(S(=O)C4CCC4)sc3n2)cn1] | 9.52 (s, 2H), 8.10 (d, J = 5.2 Hz, 1H), 7.61 (s, 1H), 7.60-7.56 (m, 3H), 7.52-7.48 (m, 2H), 4.63 (s, 2H), 4.02-3.94 (m, 1H), 3.12 (d, J = 4.8 Hz, 3H), 2.87-2.73 (m, 1H), 2.47-2.31 (m, 2H), 2.31-2.19 (m, 1H), 2.15-2.00 (m, 2H) | 464.0 | |
| 61 | 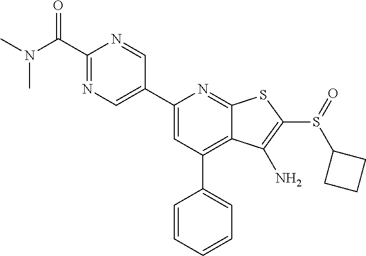 [SMILES: CN(C)C(=O)c1ncc(-c2cc(-c3ccccc3)c3c(N)c(S(=O)C4CCC4)sc3n2)cn1] | 9.48 (s, 2H), 7.61 (s, 1H), 7.60-7.54 (m, 3H), 7.51-7.49 (m, 2H), 4.63 (s, 2H), 4.02-3.94 (m, 1H), 3.19 (s, 3H), 3.00 (s, 3H), 2.86- 2.75 (m, 1H), 2.45-2.32 (m, 2H), 2.30-2.20 (m, 1H), 2.16-2.04 (m, 2H) | 478.0 | |
| 62 | 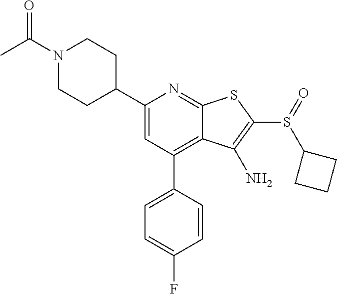 [SMILES: CC(=O)N1CCC(c2cc(-c3ccc(F)cc3)c3c(N)c(S(=O)C4CCC4)sc3n2)CC1] | 7.43-7.40 (m, 2H), 7.22 (t, J = 8.8 Hz, 2H), 6.95 (s, 1H), 4.78 (d, J = 13.2 Hz, 1H), 4.53 (d, J = 5.2 Hz, 2H), 4.0-4.0 (m, 2H), 3.25- 3.18 (m, 1H), 3.10-3.02 (m, 1H), 2.82-2.66 (m, 2H), 2.40-2.29 (m, 2H), 2.27-2.18 (m, 1H), 2.13 (s, 3H), 2.09-2.00 (m, 4H), 1.92-1.73 (m, 2H) | 472.2 | |
| 63 | 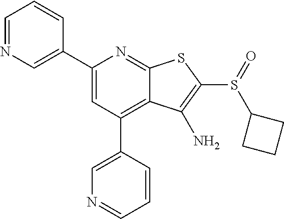 [SMILES: Nc1c(S(=O)C2CCC2)sc2nc(-c3cccnc3)cc(-c3cccnc3)c12] | 9.30 (d, J = 1.6 Hz, 1H), 8.84-8.82 (m, 2H), 8.71 (dd, J = 1.6, 4.8 Hz, 1H), 8.51-8.48 (m, 1H), 7.89 (d, J = 7.6 Hz, 1H), 7.59 (s, 1H), 7.52-7.37 (m, 2H), 4.51 (s, 2H), 4.03-3.94 (m, 1H), 2.86-2.79 (m, 1H), 2.41-2.29 (m, 5H). | 407.2 | |
| 64 | 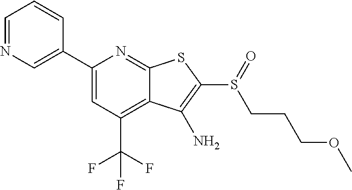 [SMILES: COCCCS(=O)c1sc2nc(-c3cccnc3)cc(C(F)(F)F)c2c1N] | 9.33 (d, J = 1.6 Hz, 1H), 8.74 (dd, J₁ = 1.6, J₁ = 4.8 Hz, 1H), 8.47- 8.44 (m, 1H), 8.06 (s, 1H), 7.50- 7.47 (m, 1H), 5.30 (s, 2H), 3.55 (t, J = 6.0 Hz, 2H), 3.47-3.40 (m, 1H), 3.34- 3.26 (s, 3H), 3.35-3.26 (m, 1H), 2.13-2.02 (m, 2H) | 416.0 | R-isomer: 100% ee; [α] = +46.5° S-isomer: 100% ee; [α] = −60.8° |
| 65 | 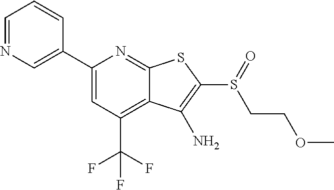 [SMILES: COCCS(=O)c1sc2nc(-c3cccnc3)cc(C(F)(F)F)c2c1N] | 9.33 (d, J = 2.0 Hz, 1H), 8.75 (dd, J₁ = 1.6, J₂ = 4.8 Hz, 1H), 8.48- 8.45 (m, 1H), 8.07 (s, 1H), 7.51-7.47 (m, 1H), 5.26 (s, 2H), 3.93-3.87 (m, 1H), 3.74-3.65 (m, 2H), 3.42 (s, 3H), 3.37-3.30 (m, 1H) | 402.0 | R-isomer: 98.7% ee; [α] = +86.9° S-isomer: 100% ee; [α] = −88.5° |
| 66 | 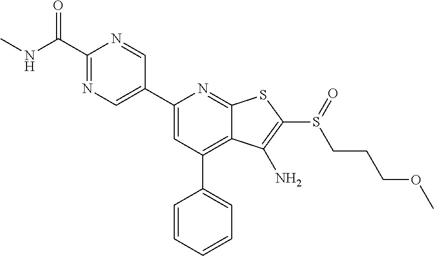 | 9.54 (s, 2H), 8.12-8.10 (m, 1H), 7.63 (s, 1H), 7.60-7.59 (m, 3H), 7.53-7.50 (m, 2H), 4.67 (s, 2H), 3.52 (t, J = 6.0 Hz, 2H), 3.41-3.36 (m, 1H), 3.35 (s, 3H), 3.29-3.22 (m, 1H), 3.13 (d, J = 4.0 Hz, 3H), 2.08-2.01 (m, 2H). | 482.1 | R-isomer: 98% ee; [α] = +60.098°; S-isomer: 98% ee; [α] = −58.480° |
| 67 | 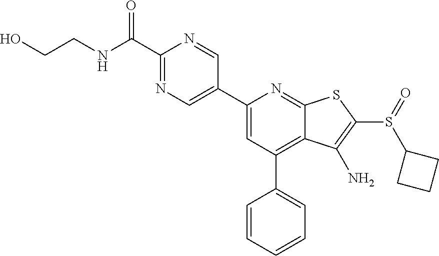 [SMILES: Nc1c(S(=O)C2CCC2)sc2nc(-c3cnc(C(=O)NCCO)nc3)cc(-c3ccccc3)c12] | 9.53 (s, 2H), 8.49 (s, 1H), 7.62-7.51 (m, 6H), 4.63 (s, 1H), 3.98-3.96 (m, 1H), 3.92-3.90 (m, 2H), 3.77-3.73 (m, 2H), 2.80-2.75 (m, 1H), 2.49-2.07 (m, 6H). | 494.1 | |
| 68 | 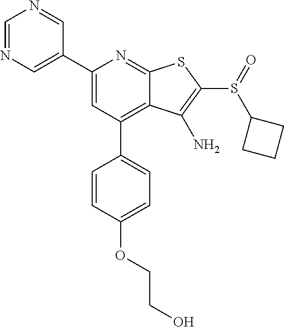 [SMILES: Nc1c(S(=O)C2CCC2)sc2nc(-c3cncnc3)cc(-c3ccc(OCCO)cc3)c12] | DMSO-d6 9.55 (s, 2H), 9.27 (s, 1H), 7.98 (s, 1H), 7.51 (d, J = 8.4 Hz, 2H), 7.16 (d, J = 8.8 Hz, 2H), 5.05 (s, 2H), 4.91 (t, J = 5.6 Hz, 1H), 4.10 (t, J = 4.8 Hz, 2H), 3.86 (t, J = 8.4 Hz, 1H), 3.79-3.75 (m, 2H), 2.64- 2.61 (m, 1H), 2.20-2.13 (m, 3H), 2.05-1.98 (m, 1H), 1.92-1.90 (m, 1H) | 467.2 | |
| 69 | 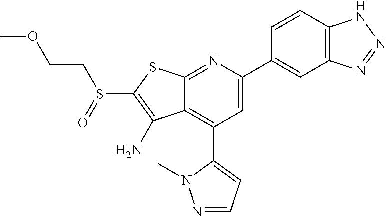 [SMILES: COCCS(=O)c1sc2nc(-c3ccc4[nH]nnc4c3)cc(-c3ccnn3C)c2c1N] | DMSO-d6 .66 (d, J = 0.8 Hz, 1H), 8.11-8.09 (m, 2H), 7.84 (d, J = 8.4 Hz, 1H), 7.69 (d, J = 1.6 Hz, 1H), 6.65 (s, 1H), 5.01 (s, 2H), 3.77-3.71 (m, 4H), 3.66-3.61 (m, 1H), 3.41- 3.35 (m, 1H), 3.28 (s, 3H), 3.25- 3.22 (m, 1H). | 454.2 | |
| 70 | 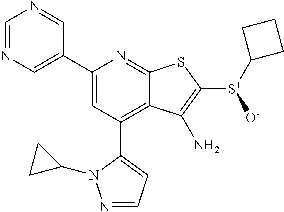 [SMILES: Nc1c([S@+]([O-])C2CCC2)sc2nc(-c3cncnc3)cc(-c3ccnn3C3CC3)c12] | 9.44 (s, 2H), 9.31 (s, 1H), 7.68 (s, 1H), 7.63 (s, 1H), 6.49 (d, J = 1.2 Hz, 1H), 4.65-4.59 (m, 2H), 3.99- 3.92 (m, 1H), 3.43-3.40 (m, 1H), 2.88-2.78 (m, 1H), 2.44-2.23 (m, 3H), 2.16-2.06 (m, 2H), 1.29-1.07 (m, 2H), 0.88-0.79 (m, 2H). | 437.1 | R-isomer: [α] = +154.740°; |
| 71 | 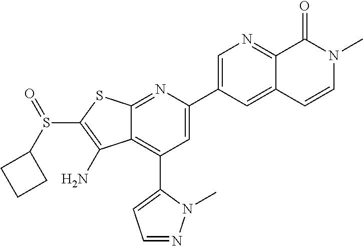 [SMILES: Cn1nccc1-c1cc(-c2cnc3c(=O)n(C)ccc3c2)nc2sc(S(=O)C3CCC3)c(N)c12] | DMSO-d6 9.57 (d, J = 2.0 Hz, 1H), 8.95 (d, J = 2.0 Hz, 1H), 8.26 (s, 1H), 7.72 (d, J = 2.0 Hz, 1H), 7.65 (d, J = 7.2 Hz, 1H), 6.73 (d, J = 7.2 Hz, 1H), 6.69 (s, 1H), 5.05 (s, 2H), 3.95-3.85 (m, 1H), 3.74 (s, 3H), 3.57 (s, 3H), 2.70-2.63 (m, 1H), 2.28- 2.11 (m, 3H), 2.10-1.99 (m, 1H), 1.99-1.87 (m, 1H). | 491.1 | R-isomer: [α] = +166.952°; |
| 72 | 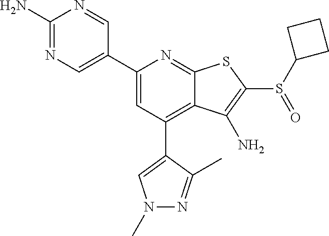 [SMILES: Cc1nn(C)cc1-c1cc(-c2cnc(N)nc2)nc2sc(S(=O)C3CCC3)c(N)c12] | 9.00 (s, 2H), 7.50 (s, 1H), 7.33 (s, 1H), 5.41 (s, 2H), 4.84 (s, 2H), 4.02-3.93 (m, 4H), 2.88-2.77 (m, 1H), 2.44-2.35 (m, 2H), 2.30-2.25 (m, 1H), 2.24 (s, 3H), 2.15-2.05 (m, 2H). | 440.0 | |
| 73 | 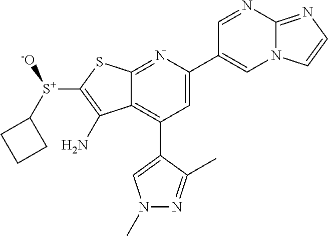 | 9.33 (d, J = 2.4 Hz, 1H), 9.15 (d, J = 2.4 Hz, 1H), 7.89 (s, 1H), 7.67 (d, J = 1.6 Hz, 1H), 7.53 (s, 1H), 7.50 (s, 1H), 4.87 (s, 2H), 3.99 (s, 3H), 3.97-3.94 (m, 2H), 2.88-2.78 (m, 1H), 2.45-2.34 (m, 2H), 2.31- 2.22 (m, 4H), 2.13-2.05 (m, 2H) | 464.0 | R-isomer: [α] = +93.331° |
| 74 | 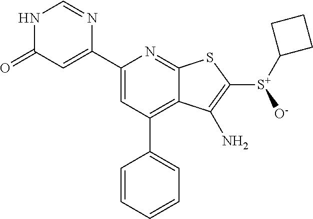 [SMILES: Nc1c([S@+]([O-])C2CCC2)sc2nc(-c3cc(=O)[nH]cn3)cc(-c3ccccc3)c12] | 8.26-8.24 (m, 1H), 8.19 (s, 1H), 7.70-7.68 (m, 1H), 7.58-7.54 (m, 3H), 7.53-7.48 (m, 2H), 4.67 (s, 2H), 4.05-3.95 (m, 1H), 2.87-2.76 (m, 1H), 2.46-2.34 (m, 2H), 2.32- 2.22 (m, 1H), 2.16-2.05 (m, 2H). | 423.1 | R-isomer: [α] = +42.7545°; |
| 75 | 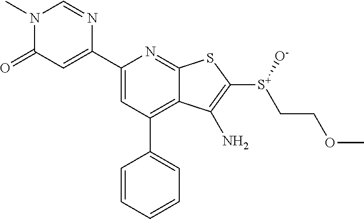 [SMILES: COCC[S@@+]([O-])c1sc2nc(-c3cc(=O)n(C)cn3)cc(-c3ccccc3)c2c1N] | 8.21 (s, 1H), 8.14 (s, 1H), 7.67-7.66 (m, 1H), 7.57-7.54 (m, 3H), 7.53-7.48 (m, 2H), 4.65 (s, 2H), 3.91-3.85 (m, 1H), 3.75-3.68 (m, 1H), 3.65-3.61 (m, 1H), 3.60 (s, 3H), 3.41 (s, 3H), 3.34-3.27 (m, 1H). | 441.2 | R-isomer: [α] = +62.306° |
| 76 | 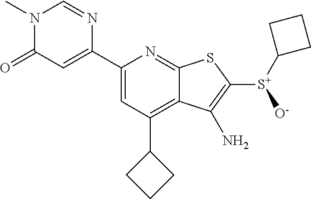 [SMILES: Cn1cnc(-c2cc(C3CCC3)c3c(N)c([S@+]([O-])C4CCC4)sc3n2)cc1=O] | 8.23 (s, 1H), 8.21 (s, 1H), 7.54 (s, 1H), 5.17 (s, 2H), 4.27-4.19 (m, 1H), 3.99-3.92 (m, 1H), 3.59 (s, 3H), 2.87-2.77 (m, 1H), 2.52-2.45 (m, 3H), 2.44-2.32 (m, 3H), 2.30- 2.20 (m, 1H), 2.19-2.13 (m, 1H), 2.12-2.04 (m, 2H), 2.03-1.95 (m, 1H). | 415.0 | R-isomer: [α] = +89.571°; |
| 77 | 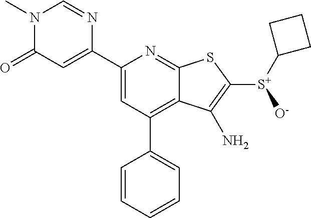 [SMILES: Cn1cnc(-c2cc(-c3ccccc3)c3c(N)c([S@+]([O-])C4CCC4)sc3n2)cc1=O] | 8.20 (s, 1H), 8.12 (s, 1H), 7.65 (s, 1H), 7.58-7.53 (m, 3H), 7.50 (s, 2H), 4.65 (s, 2H), 4.04-3.94 (m, 1H), 3.60 (s, 3H), 2.86-2.75 (m, 1H), 2.44-2.32 (m, 2H), 2.32-2.20 (m, 1H), 2.15-2.03 (m, 2H). | 437.2 | R-isomer: [α] = +163.247°; |
| 78 | 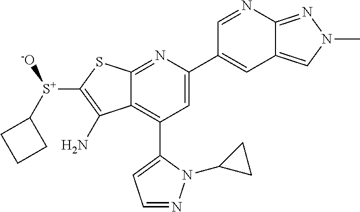 | 9.41 (d, J = 2.4 Hz, 1H), 8.80 (d, J = 2.0 Hz, 1H), 8.04 (s, 1H), 7.72 (s, 1H), 7.62 (d, J = 1.6 Hz, 1H), 6.49 (d, J = 8.0 Hz, 1H), 4.62-4.52 (m, 2H), 4.31 (s, 3H), 3.99-3.91 (m, 1H), 3.43 (s, 1H), 2.87-2.78 (m, 1H), 2.45-2.31 (m, 2H), 2.30-2.21 (m, 1H), 2.14-2.03 (m, 2H), 1.30- 1.07 (m, 2H), 0.89-0.80 (m, 2H) | 490.1 | R-isomer: [α] = +89.571°; |
| 79 | 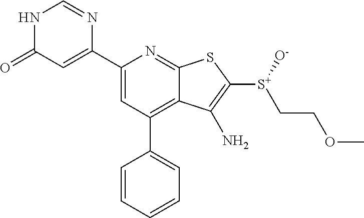 [SMILES: COCC[S@@+]([O-])c1sc2nc(-c3cc(=O)[nH]cn3)cc(-c3ccccc3)c2c1N] | | 427.1 | R-isomer: [α] = +60.503° |
| 80 | 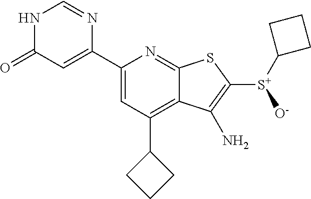 [SMILES: Nc1c([S@+]([O-])C2CCC2)sc2nc(-c3cc(=O)[nH]cn3)cc(C3CCC3)c12] | | 401.1 | R-isomer: [α] = +22.781° |
| 81 | 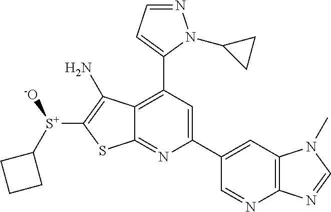 | | 490.2 | R-isomer: [α] = +153.402° |
| 82 | 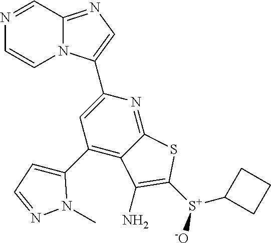 | | 450.2 | R-isomer: [α] = +214.635° |

### General Procedure B

### Step 1: Condensation to Form Hydroxypyridine

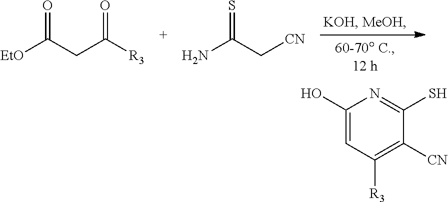

To a solution of potassium hydroxide (1.2 eq) in methanol at 60° C. was added a methanol solution of the R-keto ester (1.0 eq) and 2-cyanothioacetamide (1.0 eq). The reaction was stirred at 70° C. for 12 hours. The mixture was concentrated, dissolved in water and acidified to pH 4 with concentrated hydrochloric acid. The solid was filtered to give the target compound.

### Step 2: Alkylation

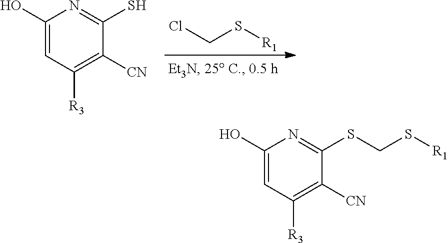

To a solution of the hydroxypyridine (1.0 eq) in acetonitrile were added triethylamine (2.0 eq) and the appropriate chloromethylsulfide (2.0 eq). The mixture was stirred at 25° C. for 15 minutes. The mixture was concentrated and the crude product was purified by prep-HPLC to give the target compound.

### Step 3: Triflation

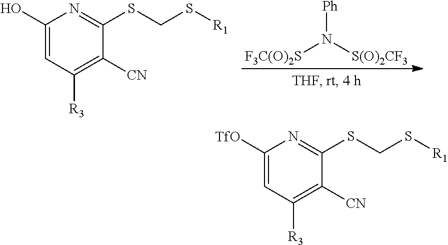

To a solution of the alkylated hydroxypyridine (1.0 eq) in tetrahydrofuran was added potassium tert-butoxide (1.2 eq) and 1,1,1-trifluoro-N-phenyl-N-(trifluoromethylsulfonyl)methanesulfonamide (1.2 eq). The mixture was stirred at 25° C. for 3 hours and concentrated. Water was added and the mixture extracted with dichloromethane. The combined organic layers were concentrated. The residue was purified by column chromatography to give the target compound.

### Step 4: Suzuki Reaction

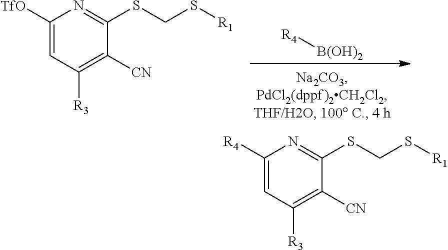

To a solution of the triflate (1.0 eq) in tetrahydrofuran and water was added sodium carbonate (2.0 eq). 1,1′-bis(diphenylphosphino)ferrocene-palladium(II) dichloride dichloromethane complex (0.05 eq) and the appropriate boronic acid (1.5 eq) were added. The reaction was stirred at 100° C. for 3 hours under nitrogen atmosphere. The mixture was concentrated and water was added. The mixture was extracted with dichloromethane and the combined organic phases were concentrated. The residue was purified by column chromatography to provide the target compound.

### Step 5: Oxidation to Sulfoxide

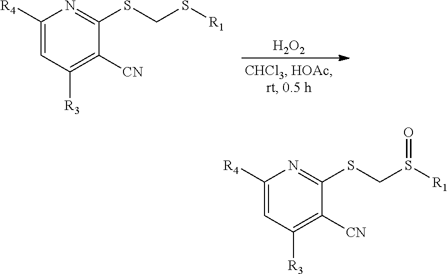

The sulfide was converted to the sulfoxide using the same procedure described in General Procedure A.

### Step 6: Cyclization

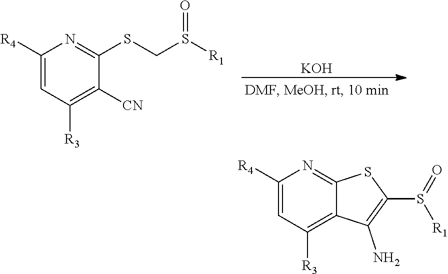

The sulfoxide was cyclized to the final target compound using the same procedure described in General Procedure A.

The following compounds were prepared from known starting materials using General Procedure B. For select compounds, the enantiomers were resolved from the racemate using chiral supercritical fluid chromatography (SFC) and are reported with enantiomeric excess (ee) and/or optical rotation values. In some cases, only the more active R-(+) isomer was prepared/isolated.

| Ex. | | 400 MHz NMR (δ; CDCl₃ | LCMS | Chiral |
|---|---|---|---|---|
| No. | Structure | unless otherwise noted) | (ES⁺; MH⁺) | info |
| 83 | 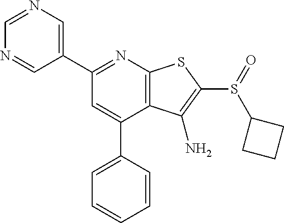 [SMILES: Nc1c(S(=O)C2CCC2)sc2nc(-c3cncnc3)cc(-c3ccccc3)c12] | 9.43 (s, 2H), 9.29 (s, 1H), 7.58 (s, 4H), 7.50 (s, 2H), 4.63 (s, 2H), 3.94-4.02 (m, 1H), 2.78-2.83 (m, 1H), 2.07-2.39 (m, 5H). | 407.1 | |
| 84 | 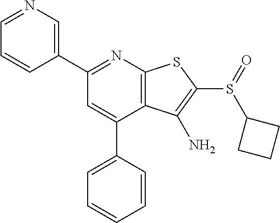 [SMILES: Nc1c(S(=O)C2CCC2)sc2nc(-c3cccnc3)cc(-c3ccccc3)c12] | 9.29 (d, J = 2.4 Hz, 1H), 8.69 (d, J = 4.0 Hz, 1H), 8.45 (d, J = 8.4 Hz, 1H), 7.59 (s, 1H), 7.56-7.45 (m, 5H), 7.43-7.42 (m, 1H), 4.60 (s, 2H), 4.02-3.93 (m, 1H), 2.82-2.77 (m, 1H), 2.38-2.25 (m, 2H), 2.10- 2.06 (m, 1H), 2.10-2.05 (m, 2H). | 406.2 | |
| 85 | 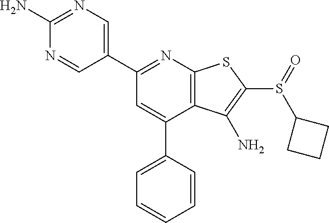 [SMILES: Nc1ncc(-c2cc(-c3ccccc3)c3c(N)c(S(=O)C4CCC4)sc3n2)cn1] | 9.03 (s, 2H), 7.49-7.55 (m, 5H), 7.41 (s, 1H), 5.32 (s, 2H), 4.57 (s, 2H), 3.93-4.01 (m, 1H), 2.77-2.82 (m, 1H), 2.35-2.38 (s, 2H), 2.25- 2.26 (m, 1H), 2.06-2.10 (m, 2H). | 422.1 | |
| 86 | 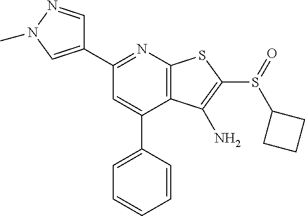 [SMILES: Cn1cc(-c2cc(-c3ccccc3)c3c(N)c(S(=O)C4CCC4)sc3n2)cn1] | 8.04 (s, 1H), 8.00 (s, 1H), 7.47-7.54 (m, 5H), 7.26 (s, 1H), 4.51 (s, 2H), 3.91- 3.98 (m, 1H), 2.76-2.83 (m, 1H), 2.23-2.38 (m, 3H), 2.05-2.09 (m, 2H). | 409.2 | |
| 87 | 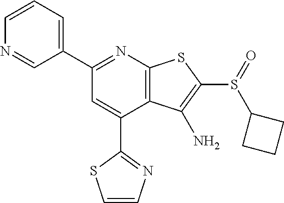 [SMILES: Nc1c(S(=O)C2CCC2)sc2nc(-c3cccnc3)cc(-c3nccs3)c12] | 9.31 (d, J = 2.0 Hz, 1H), 8.73 (d, J = 3.2 Hz, 1H), 8.476 (d, J = 8.0 Hz, 1H), 8.06 (d, J = 3.2 Hz, 1H), 7.99 (s, 1H), 7.64 (d, J = 3.2 Hz, 1H), 7.46-7.49 (m, 1H), 6.67 (s, 2H), 3.95- 4.03 (m, 1H), 2.82-2.89 (m, 1H), 2.36-2.40 (m, 2H), 2.24-2.25 (m, 1H), 2.07-2.11 (m, 2H). | 413.1 | |
| 88 | 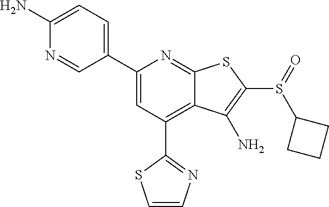 [SMILES: Nc1ccc(-c2cc(-c3nccs3)c3c(N)c(S(=O)C4CCC4)sc3n2)cn1] | 8.82 (d, J = 2.0 Hz, 1H), 8.18-8.24 (m, 3H), 8.04 (s, 1H), 6.77 (s, 1H), 6.54- 6.58 (m, 1H), 3.83-3.91 (m, 1H), 2.59-2.66 (m, 1H), 1.93-2.21 (m, 5H). | 428.1 | |
| 89 | 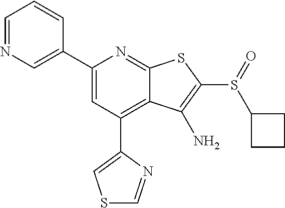 [SMILES: Nc1c(S(=O)C2CCC2)sc2nc(-c3cccnc3)cc(-c3cscn3)c12] | 9.30 (d, J = 2.0 Hz, 1H), 9.06 (d, J = 2.0 Hz, 1H), 8.70 (d, J₁ = 1.6 Hz, J₂ = 4.8 Hz, 1H), 8.46-8.49 (m, 1H), 7.82-7.84 (m, 2H), 7.45 (d, J₁ = 4.8 Hz, J₂ = 8.0 Hz, 1H), 5.93 (s, 2H), 3.93- 4.01 (m, 1H), 2.81-2.88 (m, 1H), 2.35-2.43 (m, 2H), 2.23-2.25 (m, 1H), 2.06- 2.10 (m, 2H). | 413.2 | |
| 90 | 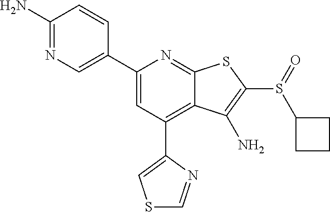 [SMILES: Nc1ccc(-c2cc(-c3cscn3)c3c(N)c(S(=O)C4CCC4)sc3n2)cn1] | 9.46 (d, J = 2.0 Hz, 1H), 8.84 (d, J = 2.4 Hz, 1H), 8.56 (d, J = 2.0 Hz, 1H), 8.23 (dd, J₁ = 8.8 Hz, J₂ = 2.4 Hz, 1H), 7.98 (s, 1H), 6.57 (d, J = 8.8 Hz, 1H), 6.46 (s, 2H), 6.37 (s, 2H), 3.82- 3.90 (m, 1H), 2.58-2.65 (m, 1H), 2.14-2.22 (m, 3H), 1.92-2.03 (m, 2H). | 428.2 | |
| 91 | 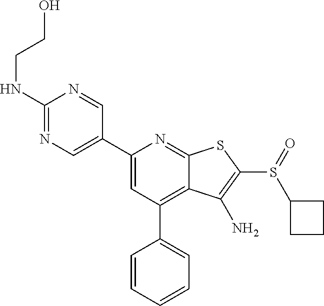 [SMILES: Nc1c(S(=O)C2CCC2)sc2nc(-c3cnc(NCCO)nc3)cc(-c3ccccc3)c12] | 9.00 (s, 2H), 7.55-7.58 (m, 4H), 7.47 (s, 1H), 7.38 (s, 1H), 5.85 (s, 1H), 4.57 (s, 2H), 3.69-3.98 (m, 5H), 2.77-2.79 (m, 1H), 2.37- 2.40 (m, 2H), 2.34-2.37 (m, 1H), 2.07-2.11 (m, 2H). | 466.3 | R-isomer: 100% ee; [α] = +3.2° S-isomer: 95.2% ee; [α] = −0.7° |
| 92 | 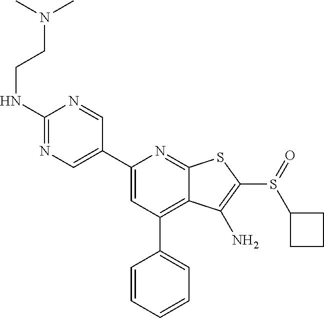 [SMILES: CN(C)CCNc1ncc(-c2cc(-c3ccccc3)c3c(N)c(S(=O)C4CCC4)sc3n2)cn1] | 8.45 (d, J = 4.9 Hz, 2H), 7.55 (s, 1H), 7.52-7.46 (m, 5H), 6.78 (t, J = 4.8 Hz, 1H), 4.64-4.55 (m, 2H), 4.49 (s, 2H), 3.99-3.89 (m, 1H), 2.85-2.71 (m, 3H), 2.48- 2.28 (m, 8H), 2.28-2.16 (m, 2H), 2.13-2.00 (m, 2H). | 493.2 | |
| 93 | 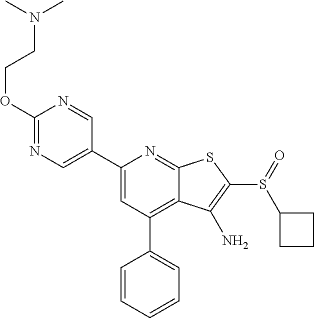 [SMILES: CN(C)CCOc1ncc(-c2cc(-c3ccccc3)c3c(N)c(S(=O)C4CCC4)sc3n2)cn1] | 9.21 (s, 2H), 7.56-7.48 (m, 6H), 4.58 (t, J = 5.6 Hz, 2H), 3.99-3.93 (m, 1H), 2.84-2.79 (m, 3H), 2.38-2.35 (m, 8H), 2.10-2.06 (m, 3H). | 494.2 | |
| 94 | 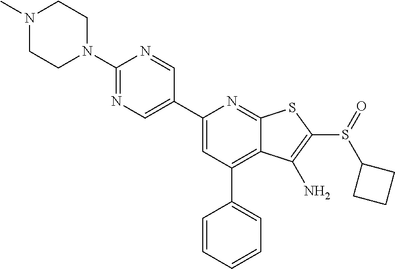 [SMILES: CN1CCN(c2ncc(-c3cc(-c4ccccc4)c4c(N)c(S(=O)C5CCC5)sc4n3)cn2)CC1] | 9.02 (s, 2H), 7.55 (m, 3H), 7.48 (m, 2H), 7.38 (s, 1H), 4.54 (s, 2H), 4.02-3.92 (m, 5H), 2.85-2.72 (m, 1H), 2.52 (m, 4H), 2.42-2.31 (m, 5H), 2.29-2.19 (m, 1H), 2.14-2.03 (m, 2H). | 505.3 | R-isomer: [α] = +57.4° S-isomer: [α] = −35.0° |
| 95 | 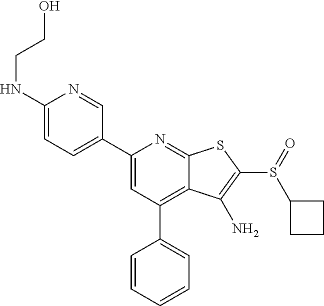 [SMILES: Nc1c(S(=O)C2CCC2)sc2nc(-c3ccc(NCCO)nc3)cc(-c3ccccc3)c12] | 8.72 (d, J = 2.0 Hz, 1H), 8.24 (dd, J₁ = 2.0 Hz, J₂ = 11.2 Hz, 1H), 7.54-7.49 (m, 5H), 7.14 (s, 1H), 6.57 (d, J = 8.8 Hz, 1H), 5.24 (s, 1H), 5.54 (s, 2H), 3.97- 3.93 (m, 1H), 3.65 (t, J = 4.8 Hz, 2H), 3.62-3.58 (m, 2H), 2.78-2.76 (m, 1H), 2.38- 2.33 (m, 2H), 2.23 (s, 1H), 2.09-2.04 (m, 2H). | 465.0 | |
| 96 | 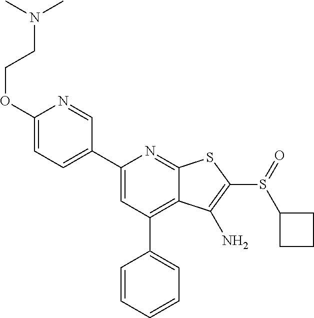 [SMILES: CN(C)CCOc1ccc(-c2cc(-c3ccccc3)c3c(N)c(S(=O)C4CCC4)sc3n2)cn1] | 8.83 (d, J = 2.4 Hz, 1H), 8.34 (dd, J₁ = 2.4 Hz, J₂ = 8.8 Hz, 1H), 7.55-7.49 (m, 6H), 6.90 (d, J = 8.8 Hz, 1H), 4.56 (s , 2H), 4.51 (t, J = 6 Hz, 1H), 4.01-3.93 (m, 1H), 2.82-2.76 (m, 2H), 2.40-2.35 (m, 8H), 2.24- 2.05 (m, 3H). | 493.0 | |
| 97 | 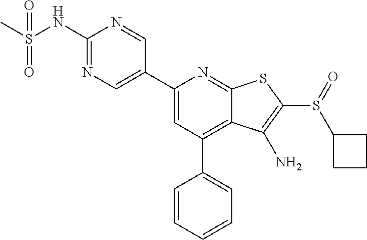 [SMILES: CS(=O)(=O)Nc1ncc(-c2cc(-c3ccccc3)c3c(N)c(S(=O)C4CCC4)sc3n2)cn1] | DMSO-d₆ 9.19 (s, 2H), 7.18 (s, 1H), 7.58-7.61 (m, 5H), 4.91 (s, 1H), 3.81-3.89 (m, 1H), 3.15 (s, 3H), 1.61-2.66 (m, 1H), 2.14-2.21 (m, 2H), 2.01-2.03 (m, 1H), 1.91-1.94 (m, 2H). | 500.1 | R-isomer: 98.2% ee; [α] = +47.6° S-isomer: 93.0% ee; [α] = −66.1° |
| 98 | 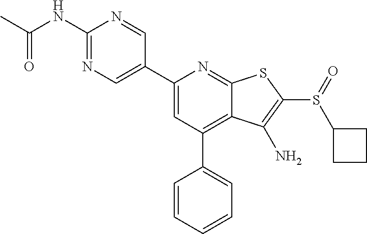 [SMILES: CC(=O)Nc1ncc(-c2cc(-c3ccccc3)c3c(N)c(S(=O)C4CCC4)sc3n2)cn1] | 9.27 (s, 2H), 8.23 (brs, 1H), 7.50-7.58 (m, 6H), 4.60 (s, 2H), 3.94-4.02 (m, 1H), 2.55-2.85 (m, 1H), 2.38 (s, 3H), 2.34-2.36 (m, 2H), 2.24-2.25 (m, 1H), 2.07-2.11 (m, 2H). | 464.2 | |
| 99 | 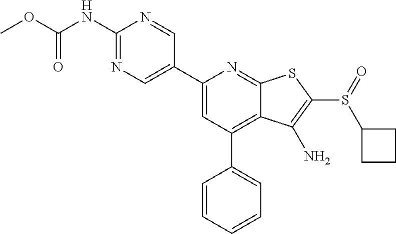 [SMILES: COC(=O)Nc1ncc(-c2cc(-c3ccccc3)c3c(N)c(S(=O)C4CCC4)sc3n2)cn1] | 9.29 (s, 2H), 9.02 (brs, 1H), 7.44-7.57 (m, 6H), 4.60 (s, 2H), 3.91-3.99 (m, 1H), 3.88 (s, 3H), 2.78-2.83 (m, 1H), 2.24-2.38 (m, 3H), 2.04-2.10 (m, 2H). | 480.1 | |
| 100 | 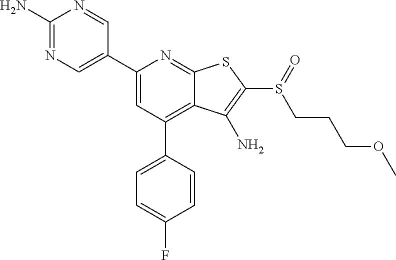 [SMILES: COCCCS(=O)c1sc2nc(-c3cnc(N)nc3)cc(-c3ccc(F)cc3)c2c1N] | 9.03 (s, 2H), 7.50-7.47 (m, 2H), 7.39 (s, 1H), 7.28- 7.27 (m, 1H), 7.25-7.23 (m, 1H), 5.34 (s, 2H), 4.58 (s, 2H), 3.53-3.50 (m, 2H), 3.40-3.30 (m, 4H), 3.27- 3.20 (m, 1H), 2.07-2.00 (m, 2H) | 458.2 | R-isomer: 100% ee; [α] = +29.3° S-isomer: 100% ee; [α] = −28.5° |
| 101 | 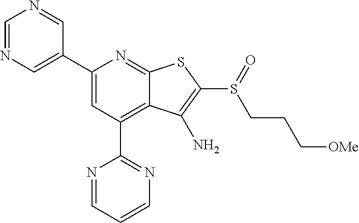 [SMILES: COCCCS(=O)c1sc2nc(-c3cncnc3)cc(-c3ncccn3)c2c1N] | 9.51 (s, 2H), 9.31 (s, 1H), 9.01 (d, J = 4.8 Hz, 2H), 8.51 (s, 1H), 7.49 (t, J = 4.8 Hz, 1H), 6.50 (s, 2H), 3.53 (t, J = 5.6 Hz, 1H), 4.43-3.36 (m, 1H), 3.36 (s, 3H), 3.31-3.22 (m, 1H), 2.09-2.02 (m, 2H). | 427.0 | R-isomer: 100% ee; [α] = +84.6° S-isomer: 100% ee; [α] = −160.5° |
| 102 | 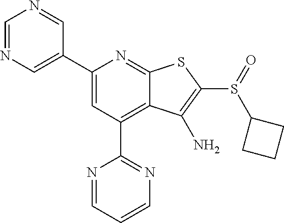 [SMILES: Nc1c(S(=O)C2CCC2)sc2nc(-c3cncnc3)cc(-c3ncccn3)c12] | 9.52 (s, 2H), 9.32 (s, 1H), 9.02 (d, J = 4.8 Hz, 2H), 8.50 (s, 1H), 7.51 (t, J = 4.8 Hz, 1H), 4.05-3.96 (m, 1H), 2.89-2.81 (m, 1H), 2.50-2.06 (m, 5H). | 409.0 | |
| 103 | 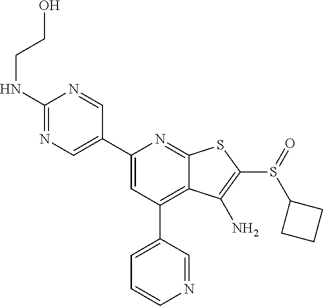 [SMILES: Nc1c(S(=O)C2CCC2)sc2nc(-c3cnc(NCCO)nc3)cc(-c3cccnc3)c12] | 8.97 (s, 2H), 8.81 (dd, J₁ = 1.6, J₂ = 4.8 Hz, 2H), 7.86 (s, 1H), 7.52-7.49 (m, 1H), 7.34 (s, 1H), 5.91 (t, J = 6.0 Hz, 1H), 4.46 (s, 2H), 3.95-3.87 (m, 3H), 3.74- 3.62 (m, 3H), 2.83-2.74 (m, 1H), 2.42-2.32 (m, 2H), 2.26-2.18 (m, 1H), 2.13- 2.05 (m, 2H) | 467.0 | R-isomer: 100% ee; [α] = +83.4° S-isomer: 96.5% ee; [α] = −71.0° |
| 104 | 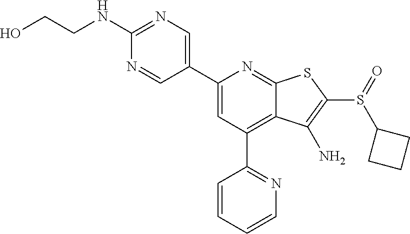 [SMILES: Nc1c(S(=O)C2CCC2)sc2nc(-c3cnc(NCCO)nc3)cc(-c3ccccn3)c12] | 9.03 (s, 2H), 8.77 (d, J = 4 Hz, 1H), 7.97-7.95 (m, 1H), 7.72 (d, J = 8 Hz, 1H), 7.53 (s, 1H), 7.50-7.49 (m, 1H), 5.83 (t, J = 6 Hz, 1H), 5.58 (s, 2H), 3.96- 3.88 (m, 3H), 3.71-3.69 (m, 2H), 3.41 (s, 1H), 2.82- 2.79 (m, 1H), 2.37-2.33 (m, 2H), 2.09-2.05 (m, 3H). | 467.0 | R-isomer: 98.5% ee; [α] = +128.3° S-isomer: 95.8% ee; [α] = −121.9° |
| 105 | 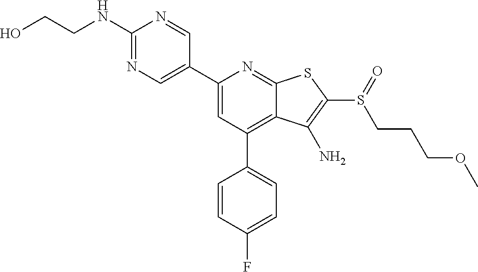 [SMILES: COCCCS(=O)c1sc2nc(-c3cnc(NCCO)nc3)cc(-c3ccc(F)cc3)c2c1N] | 8.99 (s, 2H), 7.52-7.47 (m, 2H), 7.34 (s, 1H), 7.31- 7.27 (m, 1H), 7.27-7.21 (m, 1H), 6.01 ( t, J = 5.8 Hz, 1H), 4.57 (br s, 2H), 3.89- 3.87 (m, 2H), 3.70-3.69 (m, 2H), 3.52-3.49 (m, 2H), 3.33 (s, 4H), 3.22-3.16 (m, 1H), 2.05-2.00 (m, 2H) | 502.1 | |
| 106 | 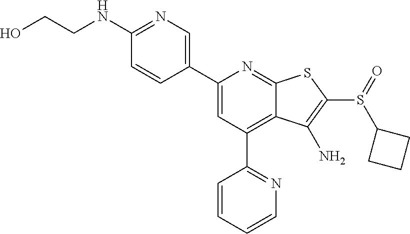 [SMILES: Nc1c(S(=O)C2CCC2)sc2nc(-c3ccc(NCCO)nc3)cc(-c3ccccn3)c12] | 8.76 (s, 2H), 8.27 (dd, J₁ = 2.4 Hz, J₂ = 8.8 Hz, 1H), 7.95-7.96 (m, 1H), 7.73 (d, J = 8 Hz, 1H), 7.58 (s, 1H), 7.47-7.49 (m, 1H), 6.57 (d, J = 8.8 Hz, 1H), 5.60 (s, 2H), 5.15 (m, 1H), 3.92-3.96 (m, 1H), 3.86 (t, J = 4.8 Hz, 2H), 3.59- 3.62 (m, 2H), 2.78-2.83 (m, 1H), 2.36-2.39 (m, 2H), 2.05-2.09 (m, 3H). | 466.0 | R-isomer: 100% ee; [α] = +21.0° S-isomer: 100% ee; [α] = −52.9° |
| 107 | 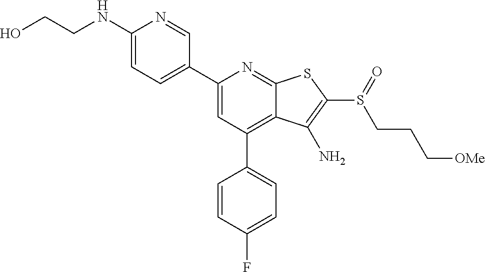 [SMILES: COCCCS(=O)c1sc2nc(-c3ccc(NCCO)nc3)cc(-c3ccc(F)cc3)c2c1N] | 8.58 (d, J = 2.0 Hz, 1H), 8.18 (dd, J₁ = 2.0, J₁ = 8.8 Hz, 1H), 7.59-7.37 (m, 2H), 7.29 (s, 2H), 7.26 (s, 1H), 6.53 (d, J = 3.6 Hz, 1H), 5.36 (t, J = 6.4 Hz, 1H), 4.54 (s, 2H), 3.90- 3.80 (m, 2H), 3.57-3.55 (m, 2H), 3.52-3.47 (m, 2H), 3.36-3.29 (m, 4H), 3.21- 3.14 (m, 1H), 2.02-1.95 (m, 2H) | 501.0 | |
| 108 | 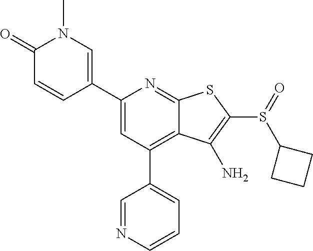 [SMILES: Cn1cc(-c2cc(-c3cccnc3)c3c(N)c(S(=O)C4CCC4)sc3n2)ccc1=O] | DMSO-d₆ 8.81-8.74 (m, 3H), 8.31 (dd, J = 2.0, 9.2 Hz, 1H), 8.01-7.99 (m, 1H), 7.77 (s, 1H), 7.59 (dd, J = 4.8, 7.6 Hz, 1H), 6.52 (d, J = 9.6 Hz, 1H), 4.89 (s, 2H), 3.89-3.81 (m, 1H), 3.54 (s, 3H), 2.64-2.61 (m, 1H), 2.24-2.12 (m, 3H), 2.08- 1.97 (m, 1H), 1.96-1.86 (m, 1H) | 437.1 | |
| 109 | 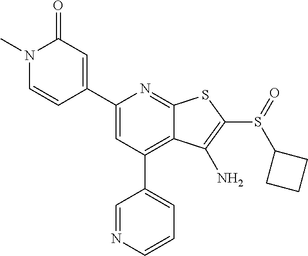 [SMILES: Cn1ccc(-c2cc(-c3cccnc3)c3c(N)c(S(=O)C4CCC4)sc3n2)cc1=O] | DMSO-d₆ 8.79 (d, J = 2.0 Hz, 1H), 8.77-8.72 (m, 1H), 8.03-8.01 (m, 1H), 7.96 (s, 1H), 7.83 (d, J = 7.2 Hz, 1H), 7.59 (dd, J = 4.8, 7.2 Hz, 1H), 7.25 (d, J = 1.6 Hz, 1H), 7.04 (dd, J = 1.6, 7.2 Hz, 1H), 4.95 (s, 2H), 3.92-3.84 (m, 1H), 3.48 (s, 3H), 2.64-2.62 (m, 1H), 2.26-2.10 (m, 3H), 2.09-1.97 (m, 1H), 1.97- 1.86 (m, 1H) | 437.1 | |
| 110 | 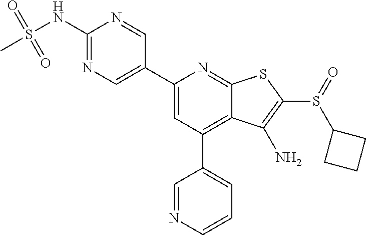 [SMILES: CS(=O)(=O)Nc1ncc(-c2cc(-c3cccnc3)c3c(N)c(S(=O)C4CCC4)sc3n2)cn1] | DMSO-d₆ 9.29 (s, 2H), 8.79 (d, J = 1.6 Hz, 1H), 8.75 (d, J = 4.0 Hz, 1H), 8.02 (d, J = 8.0 Hz, 1H), 7.94 (s, 1H), 7.61-7.58 (m, 1H), 4.93 (s, 2H), 3.91-3.83 (m, 1H), 3.27 (s, 3H), 2.66- 2.61 (m, 1H), 2.18-2.16 (m, 3H), 2.07-1.98 (m, 1H), 1.95-1.89 (m, 1H) | 501.0 | |
| 111 | 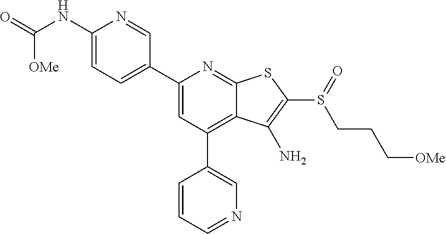 [SMILES: COCCCS(=O)c1sc2nc(-c3ccc(NC(=O)OC)nc3)cc(-c3cccnc3)c2c1N] | 8.99 (d, J = 1.6 Hz, 1H), 8.85-8.79 (m, 2H), 8.45 (dd, J = 2.0, 8.4 Hz, 1H), 8.13 (d, J = 8.8 Hz, 1H), 7.88 (d, J = 8.0 Hz, 1H), 7.55-7.49 (m, 2H), 4.52 (s, 2H), 3.85 (s, 3H), 3.53 (t, J = 6.0 Hz, 2H), 3.42-3.35 (m, 1H), 3.35 (s, 3H), 3.29-3.21 (m, 1H), 2.09-2.00 (m, 2H) | 498.2 | |
| 112 | 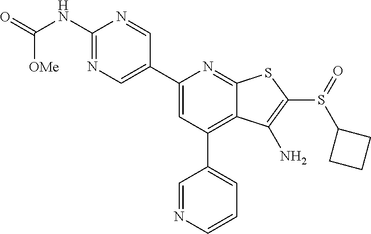 [SMILES: COC(=O)Nc1ncc(-c2cc(-c3cccnc3)c3c(N)c(S(=O)C4CCC4)sc3n2)cn1] | 9.28 (s, 2H), 8.84-8.83 (m, 1H), 8.80 (d, J = 1.6 Hz, 1H), 7.89-7.85 (m, 2H), 7.54-7.50 (m, 1H), 7.47 (s, 1H), 4.50 (br s, 2H), 3.99- 3.95 (m, 1H), 3.88 (s, 3H), 2.86-2.76 (m, 1H), 2.45- 2.32 (m, 2H), 2.29-2.25 (m, 1H), 2.12-2.06 (m, 2H) | 481.1 | |
| 113 | 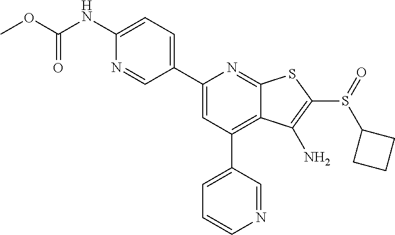 [SMILES: COC(=O)Nc1ccc(-c2cc(-c3cccnc3)c3c(N)c(S(=O)C4CCC4)sc3n2)cn1] | 8.98 (s, 1H), 8.82-8.81 (m, 2H), 8.46 (br d, J = 8.4 Hz, 1H), 8.14 (d, J = 8.8 Hz, 1H), 7.87 (br d, J = 4.6 Hz, 1H), 7.55-7.47 (m, 2H), 4.49 (br s, 2H), 3.96 (t, J = 8.0 Hz, 1H), 3.84 (s, 3H), 2.82-2.80 (m, 1H), 2.39- 2.34 (m, 2H), 2.11-2.10 (m, 1H), 2.09-2.07 (m, 2H) | 480.1 | |
| 114 | 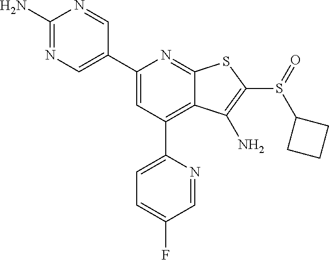 [SMILES: Nc1ncc(-c2cc(-c3ccc(F)cn3)c3c(N)c(S(=O)C4CCC4)sc3n2)cn1] | 9.04 (s, 2H), 8.64 (d, J = 2.8 Hz, 1H), 7.78-7.73 (m, 1H), 7.72-7.65 (m, 1H), 7.52 (s, 1H), 5.44 (s, 2H), 5.36 (s, 2H), 3.98-3.90 (m, 1H), 2.88-2.76 (m, 1H), 2.45-2.29 (m, 2H), 2.26- 2.21 (m, 1H), 2.15-2.01 (m, 2H) | 441.0 | |
| 115 | 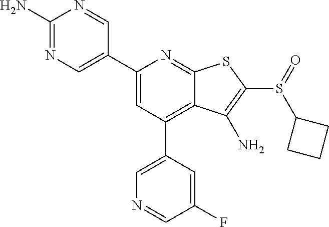 [SMILES: Nc1ncc(-c2cc(-c3cncc(F)c3)c3c(N)c(S(=O)C4CCC4)sc3n2)cn1] | DMSO-d₆ 9.06 (s, 2H), 8.74 (d, J = 2.8 Hz, 1H), 8.64-8.62 (m, 1H), 8.07-7.97 (m, 1H), 7.87 (s, 1H), 7.18 (s, 2H), 5.01 (s, 2H), 3.89-3.81 (m, 1H), 2.63-2.61 (m, 1H), 2.26-2.10 (m, 3H), 2.08- 1.98 (m, 1H), 1.96-1.90 (m, 1H) | 441.1 | |
| 116 | 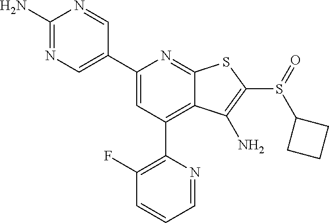 [SMILES: Nc1ncc(-c2cc(-c3ncccc3F)c3c(N)c(S(=O)C4CCC4)sc3n2)cn1] | DMSO-d₆ 9.05 (s, 2H), 8.71-8.64 (m, 1H), 8.12-8.02 (m, 2H), 7.77-7.73 (m, 1H), 7.19 (s, 2H), 5.23 (s, 2H), 3.91- 3.83 (m, 1H), 2.67-2.60 (m, 1H), 2.25-2.11 (m, 3H), 2.08-1.97 (m, 1H), 1.96-1.86 (m, 1H) | 441.0 | |
| 117 | 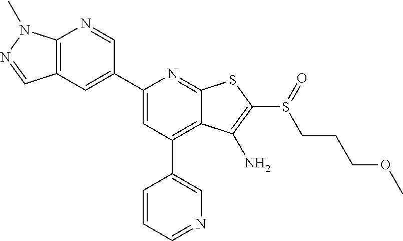 [SMILES: COCCCS(=O)c1sc2nc(-c3cnc4c(cnn4C)c3)cc(-c3cccnc3)c2c1N] | 9.29 (d, J = 2.0 Hz, 1H), 8.86-8.82 (m, 2H), 8.81 (d, J = 2.0 Hz, 1H), 8.10 (s, 1H), 7.90 (d, J = 7.6 Hz, 1H), 7.61 (s, 1H), 7.53 (dd, J = 4.8, 8.0 Hz, 1H), 4.53 (s, 2H), 4.22 (s, 3H), 3.53 (t, J = 6.0 Hz, 2H), 3.43- 3.36 (m, 1H), 3.35 (s, 3H), 3.31-3.22 (m, 1H), 2.11- 2.00 (m, 2H) | 479.1 | |
| 118 | 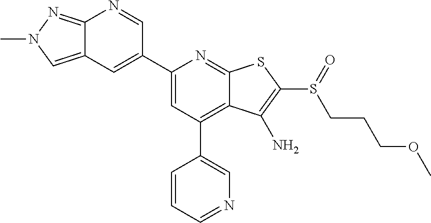 | 9.41 (d, J = 2.0 Hz, 1H), 8.85-8.80 (m, 3H), 8.04 (s, 1H), 7.90 (d, J = 7.6 Hz, 1H), 7.62 (s, 1H), 7.53 (dd, J = 5.2, 7.2 Hz, 1H), 4.52 (s, 2H), 4.31 (s, 3H), 3.53 (t, J = 6.0 Hz, 2H), 3.42-3.36 (m, 1H), 3.35 (s, 3H), 3.30-3.21 (m, 1H), 2.09- 2.01 (m, 2H) | 479.1 | |
| 119 | 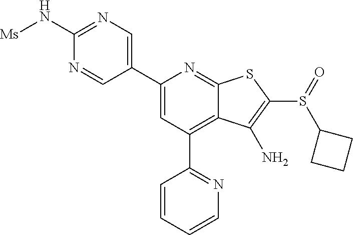 [SMILES: CS(=O)(=O)Nc1ncc(-c2cc(-c3ccccn3)c3c(N)c(S(=O)C4CCC4)sc3n2)cn1] | 9.33 (s, 2H), 8.80 (d, J = 5.6 Hz, 1H), 8.02-7.99 (m, 1H), 7.74 (d, J = 10.4 Hz, 1H), 7.64 (s, 1H), 7.53- 7.49 (m, 1H), 5.63 (s, 2H), 2.86-2.80 (m, 1H), 2.41- 2.04 (m, 5H). | 501.0 | |
| 120 | 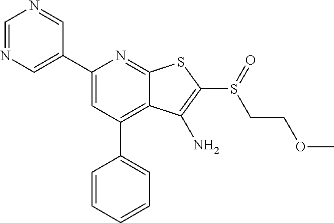 [SMILES: COCCS(=O)c1sc2nc(-c3cncnc3)cc(-c3ccccc3)c2c1N] | 9.44 (s, 2H), 9.29 (s, 1H), 7.59-7.58 (m, 4H), 7.50 (s, 2H), 4.63 (s, 2H), 3.87-3.85 (m, 1H), 3.72-3.70 (m, 1H), 3.60-3.59 (m, 1H), 3.40 (s, 3H), 3.30-3.28 (m, H) | 411.0 | R-isomer: 100% ee; [α] = +124.7° S-isomer: 100% ee; [α] = −85.5° |
| 121 | 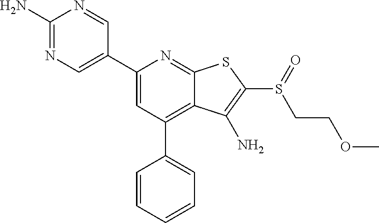 [SMILES: COCCS(=O)c1sc2nc(-c3cnc(N)nc3)cc(-c3ccccc3)c2c1N] | 9.03 (s, 2H), 7.56-7.55 (m, 3H), 7.49 (s, 2H), 7.42 (s, 1H), 5.33 (s, 2H), 4.57 (s, 2H), 3.86-3.84 (m, 1H), 3.70-3.69 (m, 1H), 3.59- 3.58 (m, 1H), 3.39 (s, 3H), 3.29-3.27 (m, 1H) | 426.0 | R-isomer: 100% ee; [α] = +49.8° S-isomer: 100% ee; [α] = −54.6° |
| 122 | 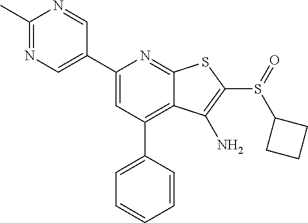 [SMILES: Cc1ncc(-c2cc(-c3ccccc3)c3c(N)c(S(=O)C4CCC4)sc3n2)cn1] | 9.31 (s, 2H), 7.57-7.50 (m, 6H), 4.61 (s, 2H), 4.02- 3.92 (m, 1H), 2.83 (s, 3H), 2.42-2.35 (m, 2H), 2.28- 2.25 (m, 1H), 2.11-2.04 (m, 2H). | 421.0 | |
| 123 | 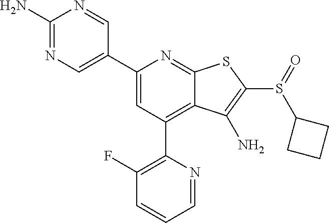 [SMILES: Nc1ncc(-c2cc(-c3ncccc3F)c3c(N)c(S(=O)C4CCC4)sc3n2)cn1] | DMSO-d₆ 9.01 (s, 2H), 8.63 (d, J = 4.0 Hz, 1H), 8.05-8.01 (m, 2H), 7.73-7.69 (m, 1H), 7.15 (s, 2H), 5.19 (s, 2H), 3.85-3.81 (t, J = 8.0 Hz, 1H), 2.64-2.57 (m, 1H), 2.19-2.08 (m, 3H), 2.01- 1.83 (m, 2H). | 441.1 | |
| 124 | 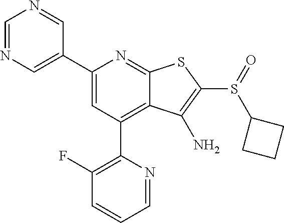 [SMILES: Nc1c(S(=O)C2CCC2)sc2nc(-c3cncnc3)cc(-c3ncccc3F)c12] | 9.44 (s, 2H), 9.31 (s, 1H), 8.64-8.63 (m, 1H), 7.80 (d, J = 2.4 Hz, 1H), 7.77-7.71 (m, 1H), 7.58-7.54 (m, 1H), 5.60-4.92 (m, 2H), 2.91- 2.72 (m, 1H), 2.52-1.93 (m, 6H). | 426.2 | |
| 125 | 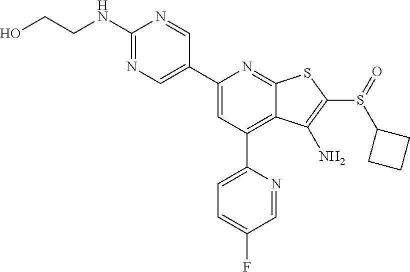 [SMILES: Nc1c(S(=O)C2CCC2)sc2nc(-c3cnc(NCCO)nc3)cc(-c3ccc(F)cn3)c12] | 9.02 (s, 2H), 8.64 (d, J = 2.8 Hz, 1H), 7.74-7.72 (m, 1H), 7.71-7.68 (m, 1H), 7.49 (s, 1H), 5.83 (s, 1H), 5.43 (s, 2H), 3.98-3.88 (m, 3H), 3.71-3.68 (m, 2H), 3.52-3.38 (m, 1H), 2.84- 2.79 m, 1H), 2.43-2.29 (m, 2H), 2.26-2.19 (m, 1H), 2.12-2.03 (m, 2H) | 485.0 | |
| 126 | 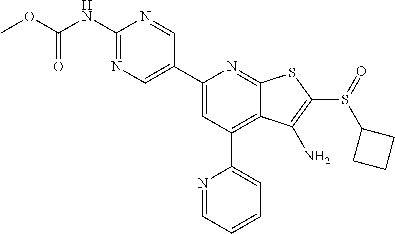 [SMILES: COC(=O)Nc1ncc(-c2cc(-c3ccccn3)c3c(N)c(S(=O)C4CCC4)sc3n2)cn1] | 9.30 (s, 2H), 8.80-8.79 (m, 1H), 8.01-7.97 (m, 2H), 7.73 (d, J = 8.0 Hz, 1H), 7.55-7.46 (m, 1H), 5.62 (s, 2H), 3.99-3.91 (m, 1H), 3.79 (s, 3H), 2.85-2.97 (s, 1H), 2.48-2.11 (m, 5H). | 427.0 | |
| 127 | 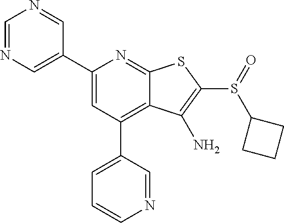 [SMILES: Nc1c(S(=O)C2CCC2)sc2nc(-c3cncnc3)cc(-c3cccnc3)c12] | 9.44 (s, 2H), 9.32 (s, 1H), 8.85-8.81 (m, 2H), 7.89- 7.87 (m, 1H), 7.59 (s, 1H), 7.55-7.51 (m, 1H), 4.54 (s, 2H), 4.03-3.93 (m, 1H), 2.86-2.77 (m, 1H), 2.45-2.23 (m, 3H), 2.15-2.06 (m, 2H). | 408.0 | |
| 128 | 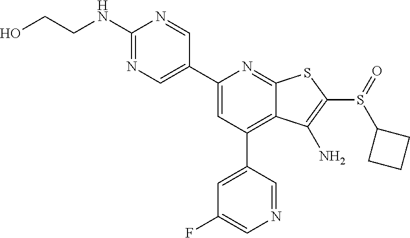 [SMILES: Nc1c(S(=O)C2CCC2)sc2nc(-c3cnc(NCCO)nc3)cc(-c3cncc(F)c3)c12] | 8.92 (s, 2H), 8.76-8.63 (m, 2H), 7.73-7.55 (m, 1H), 7.29 (s, 1H), 6.04 (t, J = 5.6 Hz, 1H), 4.45 (s, 2H), 3.98-3.82 (m, 4H), 3.76- 3.63 (m, 2H), 2.84-2.72 (m, 1H), 2.41-2.30 (m, 2H), 2.29-2.17 (m, 1H), 2.13-2.03 (m, 2H) | 485.2 | |
| 129 | 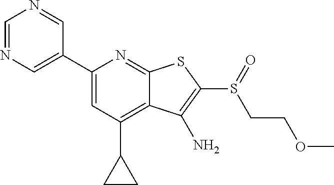 [SMILES: COCCS(=O)c1sc2nc(-c3cncnc3)cc(C3CC3)c2c1N] | 9.38 (s, 2H), 9.28 (s, 1H), 7.47 (s, 1H), 5.58 (s, 2H), 3.91-3.89 (m, 1H), 3.75- 3.73 (m, 1H), 3.67-3.61 (m, 1H), 3.43 (s, 3H), 3.35- 3.29 (m, 1H), 2.58-2.54 (m, 1H), 1.25-1.23 (m, 2H), 1.11-1.06 (m, 2H). | 375.0 | R-isomer: 94.0% ee; [α] = +78.0° S-isomer: 96.1% ee; [α] = −91.3° |
| 130 | 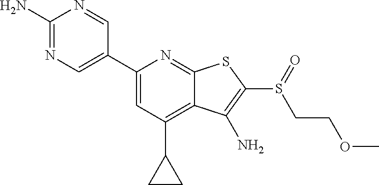 [SMILES: COCCS(=O)c1sc2nc(-c3cnc(N)nc3)cc(C3CC3)c2c1N] | 8.98 (s, 2H), 7.32 (s, 1H), 5.54 (s, 2H), 5.30 (s, 2H), 3.90-3.88 (m, 1H), 3.73- 3.71 (m, 1H), 3.63-3.61 (m, 1H), 3.43 (s, 3H), 3.30- 3.27 (m, 1H), 2.53-2.49 (m, 1H), 1.26 (s, 1H), 1.21- 1.19 (m, 2H), 1.08-1.03 (m, 2H). | 390.0 | R-isomer: 100% ee; [α] = +57.1° S-isomer: 100% ee; [α] = −22.5° |
| 131 | 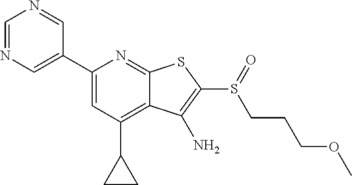 [SMILES: COCCCS(=O)c1sc2nc(-c3cncnc3)cc(C3CC3)c2c1N] | 9.37 (s, 2H), 9.28 (s, 1H), 7.46 (s, 1H), 5.62 (s, 2H), 3.55 (t, J = 4.0 Hz, 2H), 3.44-3.39 (m, 1H), 3.36 (s, 3H), 3.31-3.24 (m, 1H), 2.60-2.53 (m, 1H), 2.11-2.05 (m, 2H), 1.26-1.21 (m, 2H), 1.14-1.02 (m, 2H). | 389.0 | R-isomer: 100% ee; [α] = +72.5° S-isomer: 98.0% ee; [α] = −67.5° |
| 132 | 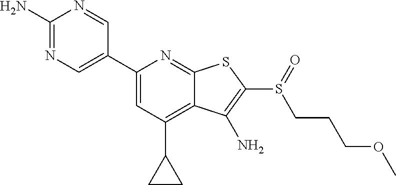 [SMILES: COCCCS(=O)c1sc2nc(-c3cnc(N)nc3)cc(C3CC3)c2c1N] | 8.98 (s, 2H), 7.31 (s, 1H), 5.57 ( s, 2H), 5.32 (s, 2H), 3.54 (t, J = 6.1 Hz, 2H), 3.44-3.37 (m, 1H), 3.36 (s, 3H), 3.31-3.22 (m, 1H), 2.55-2.48 (m, J = 5.4, 8.3 Hz, 1H), 2.11-2.02 (m, 2H), 1.23-1.16 (m, 2H), 1.12-0.98 (m, 2H). | 404.0 | R-isomer: 98.2% ee; [α] = +38.4° S-isomer: 98.9% ee; [α] = −32.8° |
| 133 | 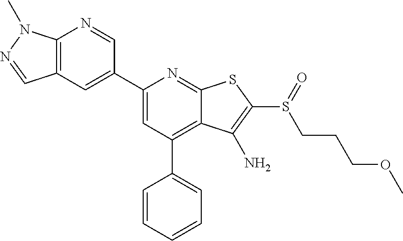 [SMILES: COCCCS(=O)c1sc2nc(-c3cnc4c(cnn4C)c3)cc(-c3ccccc3)c2c1N] | 9.29 (d, J = 2.1 Hz, 1H), 8.81 (d, J = 2.0 Hz, 1H), 8.10 (s, 1H), 7.62 (s, 1H), 7.60-7.49 (m, 5H), 4.63 (s, 2H), 4.22 (s, 3H), 3.55-3.50 (m, 2H), 3.42-3.36 (m, 1H), 3.35 (s, 3H), 3.30-3.20 (m, 1H), 2.07-2.01 (m, 2H). | 478.1 | |
| 134 | 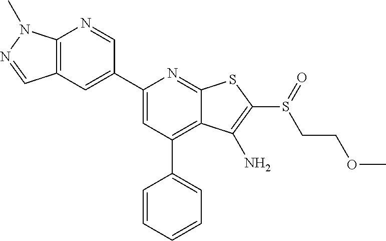 [SMILES: COCCS(=O)c1sc2nc(-c3cnc4c(cnn4C)c3)cc(-c3ccccc3)c2c1N] | 9.29 (d, J = 2.0 Hz, 1H), 8.82 (d, J = 2.0 Hz, 1H), 8.10 (s, 1H), 7.63 (s, 1H), 7.58-7.57 (m, 5H), 4.60 (s, 2H), 4.22 (s, 3H), 3.91- 3.83 (m, 1H), 3.75-3.66 (m, 1H), 3.66-3.57 (m, 1H), 3.40 (s, 3H), 3.33-3.24 (m, 1H) | 464.1 | |
| 135 | 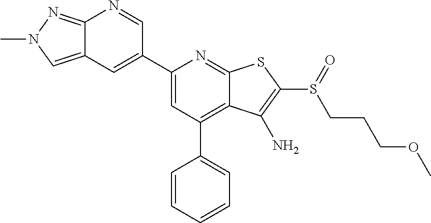 | 9.40 (d, J = 4.0 Hz, 1H), 8.78 (d, J = 4.0 Hz, 1H), 8.01 (s, 1H), 7.61 (s, 1H), 7.56-7.55 (m, 3H), 7.53- 7.51 (m, 2H), 4.62 (s, 2H), 4.30 (s, 3H), 3.51 (t, J = 5.6 Hz, 2H), 3.38-3.35 (m, 1H), 3.34 (s, 3H), 3.25- 3.23 (m, 1H), 2.06-2.00 (m, 2H). | 478.0 | |
| 136 | 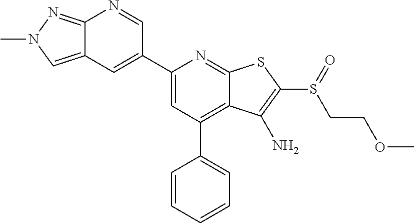 | 9.40 (d, J = 4.0 Hz, 1H), 8.82 (d, J = 4.0 Hz, 1H), 8.03 (s, 1H), 7.64 (s, 1H), 7.57-7.55 (m, 3H), 7.52 (s, 2H), 4.60 (s, 2H), 4.31 (s, 3H), 3.87-3.85 (m, 1H), 3.72-3.70 (m, 1H), 3.61- 3.60 (m, 1H), 3.40 (s, 3H), 3.31-3.29 (m, 1H). | 464.0 | R-isomer: 100% ee; [α] = +40.2 ° S-isomer: 94.6% ee; [α] = −45.4° |
| 137 | 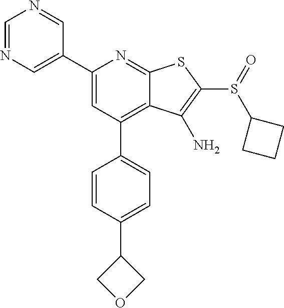 [SMILES: Nc1c(S(=O)C2CCC2)sc2nc(-c3cncnc3)cc(-c3ccc(C4COC4)cc3)c12] | DMSO-d₆ 9.55 (s, 2H), 9.27 (s, 1H), 8.03 (s, 1H), 7.65-7.60 (m, 4H), 5.05-4.97 (m, 4H), 4.71-4.68 (m, 2H), 4.44-4.34 (m, 1H), 3.86 (m, 1H), 2.65-2.57 (m, 1H), 2.24-2.10 (m, 3H), 2.06-1.96 (m, 1H), 1.91 (m, 1H) | 436.2 | |
| 138 | 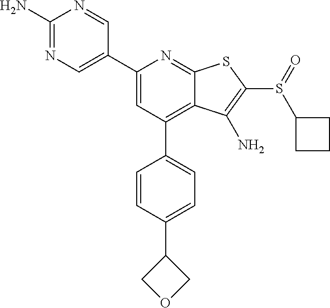 [SMILES: Nc1ncc(-c2cc(-c3ccc(C4COC4)cc3)c3c(N)c(S(=O)C4CCC4)sc3n2)cn1] | DMSO-d₆ 9.05 (s, 2H), 7.75 (s, 1H), 7.67-7.53 (m, 4H), 7.13 (s, 2H), 5.01 (m, 2H), 4.94 (s, 2H), 4.70 (t, J = 6.2 Hz, 2H), 4.39 (m, 1H), 3.84 (m, 1H), 2.72-2.54 (m, 2H), 2.18-2.10 (m, 2H), 2.07- 1.85 (m, 2H) | 478.0 | |
| 139 | 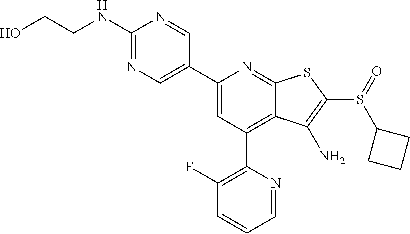 [SMILES: Nc1c(S(=O)C2CCC2)sc2nc(-c3cnc(NCCO)nc3)cc(-c3ncccc3F)c12] | 9.01 (s, 2H), 8.61 (dd, J₁ = 1.2 Hz, J₂ = 4.8 Hz, 1H), 7.70-7.68 (m, 1H), 7.60 (d, J = 2.4 Hz, 1H), 7.55-7.52 (m, 1H), 5.85 (s, 1H), 2.13 (s, 2H), 3.93-3.87 (m, 3H), 3.71-3.67 (m, 2H), 3.45-3.35 (m, 1H), 2.80-2.79 (m, 1H), 2.44-1.99 (m, 5 H). | 485.0 | R-isomer: 100% ee; [α] = +3.8° S-isomer: 97.7% ee; [α] = −69.4° |
| 140 | 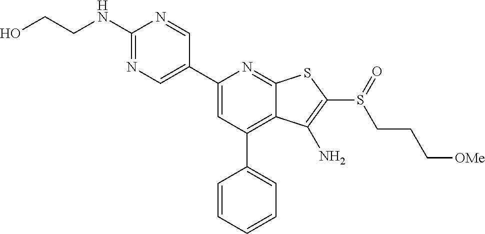 [SMILES: COCCCS(=O)c1sc2nc(-c3cnc(NCCO)nc3)cc(-c3ccccc3)c2c1N] | 8.95 (s, 2H), 7.57-7.56 (m, 4H), 7.44 (m, 1H), 7.33 (s, 1H), 5.97 (t, J = 6.0 Hz, 1H), 4.59 (s, 2H), 3.90-3.88 (m, 3H), 3.70-3.66 (m, 2H), 3.51-3.48 (m, 2H), 3.33 (s, 3H), 3.20-3.17 (m, 1H), 2.03-1.97 (m, 3H). | 484.0 | |
| 141 | 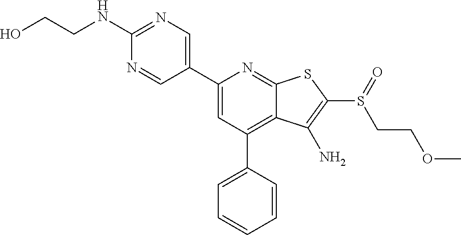 [SMILES: COCCS(=O)c1sc2nc(-c3cnc(NCCO)nc3)cc(-c3ccccc3)c2c1N] | 8.96 (s, 2H), 7.56-7.48 (m, 4H), 7.48-7.41 (m, 1H), 7.35 (s, 1H), 6.01 (t, J = 5.6 Hz, 1H), 4.57 (s, 2H), 3.94-3.80 (m, 4H), 3.70- 3.65 (m, 2H), 3.59-3.54 (m, 1H), 3.38 (s, 3H), 3.29- 3.19 (m, 1H) | 470.0 | R-isomer: 100% ee; [α] = +40.533°; S-isomer: 99% ee; [α] = −39.950°; |
| 142 | 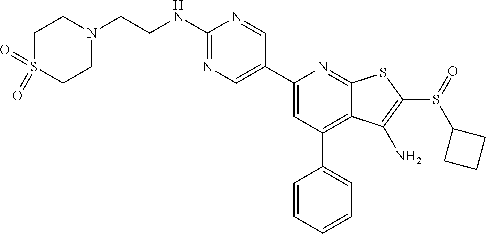 [SMILES: Nc1c(S(=O)C2CCC2)sc2nc(-c3cnc(NCCN4CCS(=O)(=O)CC4)nc3)cc(-c3ccccc3)c12] | DMSO-d₆ 9.09 (s, 2H), 7.75 (s, 1H), 7.61-7.55 (m, 5H), 4.91 (s, 2H), 3.88-3.80 (m, 1H), 3.50-3.46 (m, 2H), 3.09-3.07 (m, 4H), 2.98-2.97 (m, 4H), 2.71-2.67 (m, 2H), 2.21- 2.14 (m, 2H), 2.05-1.89 (m, 2H). | 583.3 | |
| 143 | 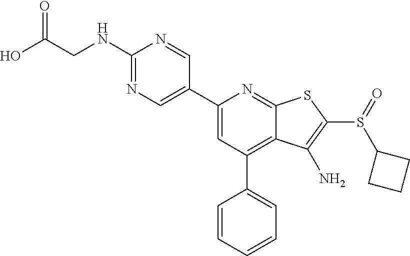 [SMILES: Nc1c(S(=O)C2CCC2)sc2nc(-c3cnc(NCC(=O)O)nc3)cc(-c3ccccc3)c12] | DMSO-d₆ 9.09 (s, 2H), 7.76 (s, 1H), 7.61-7.56 (m, 5H), 4.91 (s, 2H), 3.87-3.80 (m, 3H), 2.66-2.64 (m, 1H), 2.21-2.13 (m, 3H), 2.02-2.00 (m, 1H), 1.92-1.91 (m, 1H). | 480.1 | |
| 144 | 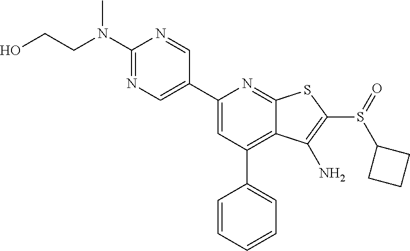 [SMILES: CN(CCO)c1ncc(-c2cc(-c3ccccc3)c3c(N)c(S(=O)C4CCC4)sc3n2)cn1] | 9.02 (s, 2H), 7.56-7.55 (m, 3H), 7.48 (s, 2H), 7.37 (s, 1H), 4.55 (s, 2H), 3.98- 3.93 (m, 3H), 3.89-3.86 (m, 2H), 3.32 (s, 3H), 2.83- 2.74 (m, 1H), 2.39-2.34 (m, 2H), 2.26-2.22 (m, 1H), 2.10-2.06 (m, 2H). | 480.1 | R-isomer: 100% ee; [α] = +80.2° S-isomer: 98.3% ee; [α] = −64.3° |
| 145 | 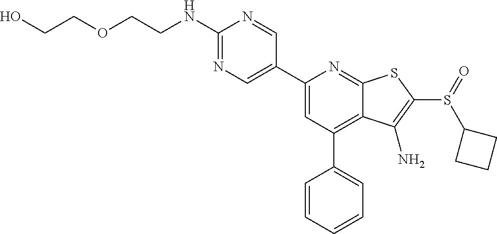 [SMILES: Nc1c(S(=O)C2CCC2)sc2nc(-c3cnc(NCCOCCO)nc3)cc(-c3ccccc3)c12] | 8.98 (s, 2H), 7.60-7.44 (m, 5H), 7.36 (s, 1H), 5.96 (s, 1H), 4.55 (s, 2H), 3.96- 3.92 (m, 1H), 3.78-3.72 (m, 6H), 3.64-3.64 (m, 2H), 2.80-2.75 (m, 1H), 2.65 (s, 1H), 2.35-2.34 (m, 2H), 2.23-2.22 (m, 1H), 2.09- 2.05 (m, 2H) | 510.2 | R-isomer: 100% ee; [α] = +86.3° S-isomer: 100% ee; [α] = −83.1° |
| 146 | 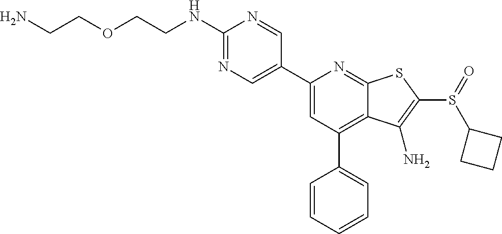 [SMILES: NCCOCCNc1ncc(-c2cc(-c3ccccc3)c3c(N)c(S(=O)C4CCC4)sc3n2)cn1] | 8.93 (s, 2H), 7.50-7.45 (m, 3H), 7.40 (m, 2H), 7.31 (m, 1H), 5.79 (m, 1H), 4.50- 4.44 (m, 2H), 3.88 (m, 1H), 3.69-3.57 (m, 4H), 3.46 (m, 2H), 2.85-2.80 (m, 2H), 2.75-2.65 (m, 1H), 2.35-2.23 (m, 2H), 2.20-2.10 (m, 1H), 2.04-1.93 (m, 2H). | 509.1 | |
| 147 | 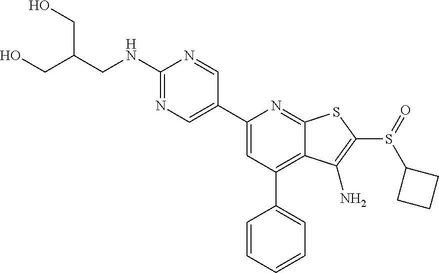 [SMILES: Nc1c(S(=O)C2CCC2)sc2nc(-c3cnc(NCC(CO)CO)nc3)cc(-c3ccccc3)c12] | DMSO-d₆ 9.08 (s, 2H), 7.75 (s, 1H), 7.60-7.56 (m, 5H), 4.90 (s, 2H), 4.47 (t, J = 5.2 Hz, 2H), 3.86-3.82 (m, 1H), 3.50-3.45 (m, 4H), 3.40- 3.36 (m, 2H), 2.73-2.68 (m, 1H), 2.18-2.14 (m, 3H), 2.03-2.00 (m, 1H), 1.87- 1.84 (m, 2H). | 510.2 | |
| 148 | 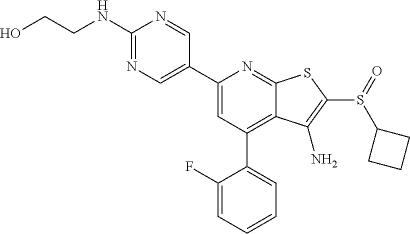 [SMILES: Nc1c(S(=O)C2CCC2)sc2nc(-c3cnc(NCCO)nc3)cc(-c3ccccc3F)c12] | DMSO-d₆ 9.07 (s, 2H), 7.84 (d, J = 1.6 Hz, 1H), 7.68-7.55 (m, 3H), 7.49-7.40 (m, 2H), 4.83 (d, J = 3.2 Hz, 2H), 4.72 (t, J = 5.6 Hz, 1H), 3.84 (t, J = 8.0 Hz, 1H), 3.54-3.52 (m, 2H), 3.44-3.41 (m, 2H), 2.64-2.58 (m, 1H), 2.17- 2.13 (m, 3H), 2.06-1.97 (m, 1H), 1.95-1.86 (m, 1H) | 484.1 | |
| 149 | 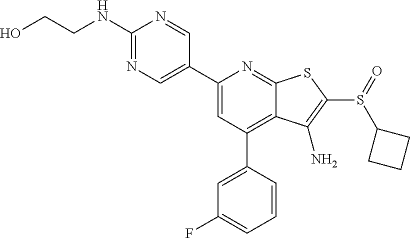 [SMILES: Nc1c(S(=O)C2CCC2)sc2nc(-c3cnc(NCCO)nc3)cc(-c3cccc(F)c3)c12] | DMSO-d₆ 9.08 (s, 2H), 7.79 (s, 1H), 7.63-7.59 (m, 2H), 7.59- 7.38 (m, 3H), 4.92 (s, 2H), 4.78-4.67 (m, 1H), 3.86- 3.81 (m, 1H), 3.53 (s, 2H), 3.44-3.39 (m, 2H), 2.66- 2.60 (m, 1H), 2.21-2.13 (m, 3H), 2.02-1.92 (m, 2H). | 484.1 | |
| 150 | 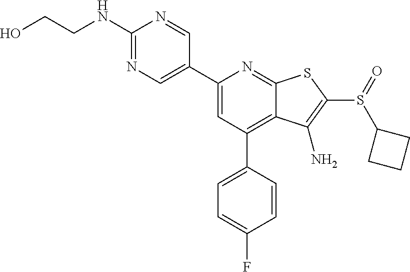 [SMILES: Nc1c(S(=O)C2CCC2)sc2nc(-c3cnc(NCCO)nc3)cc(-c3ccc(F)cc3)c12] | DMSO-d₆ 9.08 (s, 2H), 7.75 (s, 1H), 7.65-7.58 (m, 3H), 7.43 (m, 2H), 4.91 (s, 2H), 4.74 (s, 1H), 3.88-3.79 (m, 1H), 3.54 (t, J = 6.0 Hz, 2H), 3.42 (t, J = 6.2 Hz, 2H), 2.67-2.58 (m, 1H), 2.22- 2.13 (m, 3H), 2.06-1.97 (m, 1H), 1.92 (m, 1H). | 484.0 | |
| 151 | 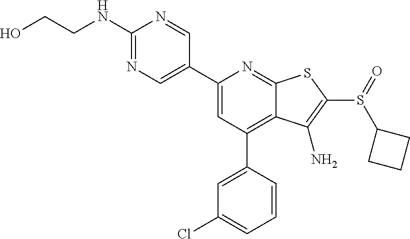 [SMILES: Nc1c(S(=O)C2CCC2)sc2nc(-c3cnc(NCCO)nc3)cc(-c3cccc(Cl)c3)c12] | DMSO-d₆ 9.09 (s, 2H), 7.80 (s, 1H), 7.70 (s, 1H), 7.68-7.56 (m, 3H), 7.56- 7.49 (m, 1H), 4.92 (s, 2H), 4.74 (t, J = 5.4 Hz, 1H), 3.91-3.78 (m, 1H), 3.58-3.50 (m, 2H), 3.42 (m, 2H), 2.70-2.58 (m, 1H), 2.23-2.10 (m, 3H), 2.09-1.97 (m, 1H), 1.97-1.86 (m, 1H) | 500.1 | |
| 152 | 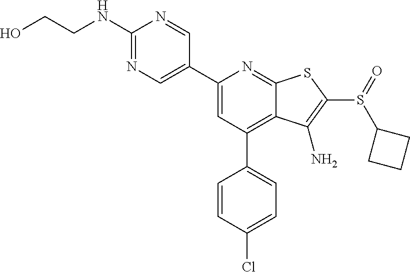 [SMILES: Nc1c(S(=O)C2CCC2)sc2nc(-c3cnc(NCCO)nc3)cc(-c3ccc(Cl)cc3)c12] | DMSO-d₆ 9.08 (s, 2H), 7.76 (s, 1H), 7.67-7.64 (m, 2H), 7.61-7.59 (m, 2H), 4.92 (s, 2H), 4.74 (t, J = 5.6 Hz, 1H), 3.89-3.80 (m, 1H), 3.54 (q, J = 6.0 Hz, 2H), 3.45-3.41 (m, 2H), 2.67- 2.60 (m, 1H), 2.23-2.11 (m, 3H), 2.06-1.98 (m, 1H), 1.97-1.87 (m, 1H) | 500.0 | |
| 153 | 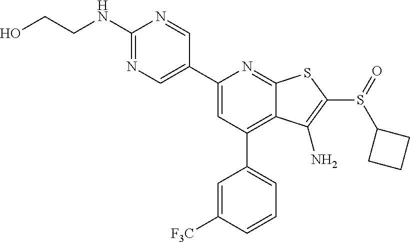 [SMILES: Nc1c(S(=O)C2CCC2)sc2nc(-c3cnc(NCCO)nc3)cc(-c3cccc(C(F)(F)F)c3)c12] | 9.01 (s, 2H), 7.86-7.68 (m, 4H), 7.37 (s, 1H), 5.85 (t, J = 6.0 Hz, 1H), 4.52-4.41 (m, 2H), 4.00-3.94 (m, 1H), 3.93-3.88 (m, 2H), 3.74-3.68 (m, 2H), 2.86-2.74 (m, 1H), 2.45-2.33 (m, 2H), 2.32-2.20 (m, 1H), 2.15- 2.05 (m, 2H) | 534.3 | |
| 154 | 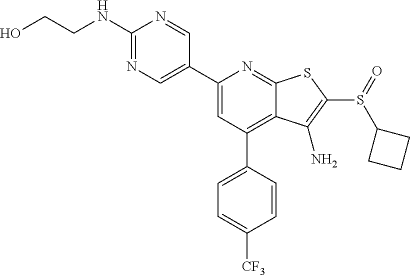 [SMILES: Nc1c(S(=O)C2CCC2)sc2nc(-c3cnc(NCCO)nc3)cc(-c3ccc(C(F)(F)F)cc3)c12] | DMSO-d₆ 9.09 (s, 2H), 7.95 (d, J = 8.4 Hz, 2H), 7.86-7.78 (m, 3H), 7.63 (t, J = 5.8 Hz, 1H), 4.87 (s, 2H), 4.74 (m, 1H), 3.85 (m, 1H), 3.54 (m, 2H), 3.42 (m, 2H), 2.69-2.61 (m, 1H), 2.23-2.10 (m, 3H), 2.09- 1.97 (m, 1H), 1.97-1.85 (m, 1H) | 534.2 | |
| 155 | 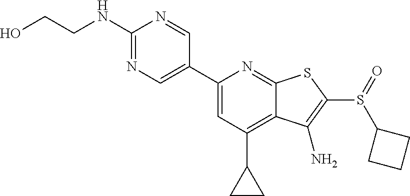 [SMILES: Nc1c(S(=O)C2CCC2)sc2nc(-c3cnc(NCCO)nc3)cc(C3CC3)c12] | DMSO-d₆ 9.03 (s, 2H), 7.56 (t, J = 5.6 Hz, 1H), 7.47 (s, 1H), 5.97 (s, 2H), 4.73 (t, J = 5.6 Hz, 1H), 3.90-3.86 (m, 1H), 3.55-3.52 (m, 2H), 3.45-3.42 (m, 2H), 2.64-2.61 (m, 2H), 2.21-2.15 (m, 3H), 2.08-1.82 (m, 2H), 1.13- 1.07 (m, 4H). | 430.1 | R-isomer: 99% ee; [α] = +165.085° S-isomer: 99% ee; [α] = −142.011° |
| 156 | 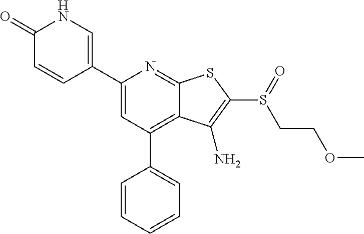 [SMILES: COCCS(=O)c1sc2nc(-c3ccc(=O)[nH]c3)cc(-c3ccccc3)c2c1N] | DMSO-d₆ 8.38 (d, J = 2.4 Hz, 1H), 8.31 (dd, J = 2.4 Hz, J = 9.6 Hz, 1H), 7.73 (s, 1H), 7.61- 7.54 (m, 5H), 6.43 (d, J = 10.0 Hz, 1H), 4.94 (s, 2H), 3.75-3.69 (m, 1H), 3.64- 3.59 (m, 1H), 3.27 (s, 3H), 3.22-3.13 (m, 2H) | 426.1 | |
| 157 | 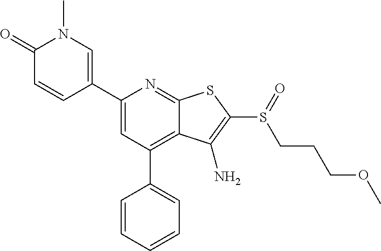 [SMILES: COCCCS(=O)c1sc2nc(-c3ccc(=O)n(C)c3)cc(-c3ccccc3)c2c1N] | DMSO-d₆ 8.76 (d, J = 2.4 Hz, 1H), 8.32 (dd, J₁ = 9.6 Hz, J₂ = 2.4 Hz, 1H), 7.73 (s, 1H), 7.62-7.57 (m, 5H), 6.52 (d, J = 9.6 Hz, 1H), 4.92 (s, 2H), 3.55 (s, 3H), 3.42 (t, J = 6.0 Hz, 2H), 3.22 (s, 3H), 3.18-3.11 (m, 1H), 3.05- 2.98 (m, 1H), 1.85-1.80 (m, 2H). | 454.2 | R-isomer: 100% ee; [α] = +42.2° S-isomer: 100% ee; [α] = −42.2° |
| 158 | 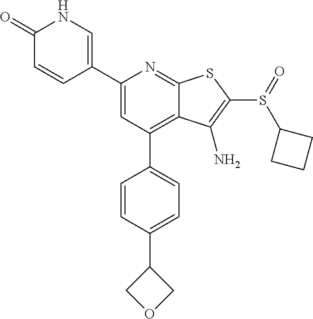 [SMILES: Nc1c(S(=O)C2CCC2)sc2nc(-c3ccc(=O)[nH]c3)cc(-c3ccc(C4COC4)cc3)c12] | DMSO-d₆ 8.35 (d, J = 2.8 Hz, 1H), 8.29 (dd, J₁ = 9.6 Hz, J₂ = 2.8 Hz, 1H), 7.70 (s, 1H), 7.63-7.55 (m, 4H), 6.43 (d, J = 9.2 Hz, 1H), 5.01 (dd, J₁ = 8.8 Hz, J₂ = 6.4 Hz, 2H), 4.93 (s, 2H), 4.69 (t, J = 6.0 Hz, 2H), 4.43-4.36 (m, 1H), 3.83 (t, J = 7.2 Hz, 1H), 2.65-2.60 (m, 1H), 2.20-2.12 (m, 3H), 2.05- 1.98 (m, 1H), 1.96-1.88 (m, 1H). | 478.3 | |
| 159 | 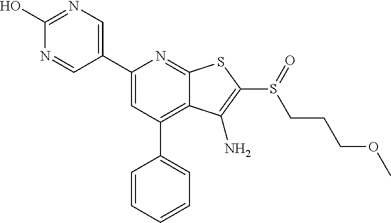 [SMILES: COCCCS(=O)c1sc2nc(-c3cnc(O)nc3)cc(-c3ccccc3)c2c1N] | DMSO-d₆ 9.38 (s, 2H), 7.95 (s, 1H), 7.61 (s, 5H), 4.95 (s, 2H), 4.69 (s, 1H), 4.01 (s, 3H), 3.49 (t, J = 6.2 Hz, 2H), 3.17 (m, 1H), 3.09-2.98 (m, 1H), 1.82-1.64 (m, 2H). | 441.1 | |
| 160 | 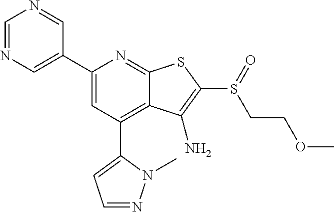 [SMILES: COCCS(=O)c1sc2nc(-c3cncnc3)cc(-c3ccnn3C)c2c1N] | 9.37 (s, 2H), 9.25 (s, 1H), 7.62 (d, J = 2.0 Hz, 1H), 7.58 (s, 1H), 6.45-6.43 (m, 1H), 4.48 (d, J = 28.8 Hz, 2H), 3.82-3.79 (m, 1H), 3.70 (s, 3H), 3.65-3.52 (m, 2H), 3.34 (s, 3H), 3.21- 3.19 (m, 1H). | 415.0 | |
| 161 | 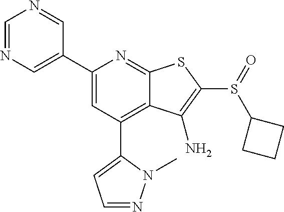 [SMILES: Cn1nccc1-c1cc(-c2cncnc2)nc2sc(S(=O)C3CCC3)c(N)c12] | 9.44 (s, 2H), 9.32 (s, 1H), 7.69 (s, 1H), 7.63 (m, 1H), 6.50 (m, 1H), 4.65-4.50 (m, 2H), 3.95 (m, 1H), 3.76 (s, 3H), 2.89-2.78 (m, 1H), 2.48-2.22 (m, 3H), 2.17- 2.04 (m, 2H) | 411.2 | R-isomer: 100% ee; [α] = +155.822°; S-isomer: 99% ee; [α] = −152.807°; |
| 162 | 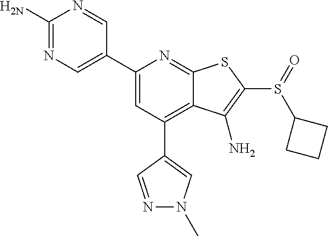 [SMILES: Cn1cc(-c2cc(-c3cnc(N)nc3)nc3sc(S(=O)C4CCC4)c(N)c23)cn1] | DMSO-d₆ 9.02 (s, 2H), 8.15 (s, 1H), 7.81 (s, 1H), 7.72 (s, 1H), 7.12 (s, 2H), 5.36 (s, 2H), 3.95 (s, 3H), 3.89-3.81 (m, 1H), 2.68-2.59 (m, 1H), 2.23- 2.14 (m, 3H), 2.07-1.98 (m, 1H), 1.96-1.88 (m, 1H) | 426.2 | R-isomer: 100% ee; [α] = +147.330°; S-isomer: 100% ee; [α] = −115.373°; |
| 163 | 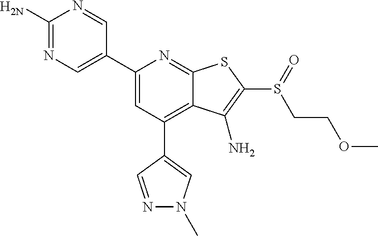 [SMILES: COCCS(=O)c1sc2nc(-c3cnc(N)nc3)cc(-c3cnn(C)c3)c2c1N] | DMSO-d₆ 9.03 (s, 2H), 8.15 (s, 1H), 7.81 (s, 1H), 7.73 (s, 1H), 7.12 (s, 2H), 5.37 (s, 2H), 3.95 (s, 3H), 3.76-3.70 (m, 1H), 3.64-3.60 (m, 1H), 3.40-3.36 (m, 1H), 3.28 (s, 3H), 3.22-3.17 (m, 1H). | 430.2 | |
| 164 | 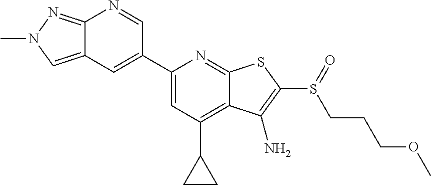 | 9.36 (d, J = 2.0 Hz, 1H), 8.75 (d, J = 2.0 Hz, 1H), 8.03 (s, 1H), 7.52 (s, 1H), 5.60 (s, 2H), 4.32 (s, 3H), 3.58-3.54 (m, 2H), 3.45- 3.39 (m, 1H), 3.38 (s, 3H), 3.33-3.24 (m, 1H), 2.61- 2.51 (m, 1H), 2.13-2.05 (m, 2H), 1.25-1.18 (m, 2H), 1.17-1.03 (m, 2H) | 442.1 | R-isomer: 99.1% ee; [α] = +90.130°; S-isomer: 98.6% ee; [α] = −91.799°; |
| 165 | 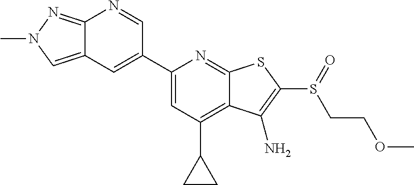 | 9.33 (d, J = 4.0 Hz, 1H), 8.74 (d, J = 4.0 Hz, 1H), 8.01 (s, 1H), 7.51 (s, 1H), 5.54 (s, 2H), 4.30 (s, 3H), 3.91-3.89 (m, 2H), 3.74- 3.70 (m, 1H), 3.66-3.61 (m, 1H), 3.43 (s, 3H), 3.34- 3.28 (m, 1H), 2.55-2.50 (m, 1H), 1.23-1.20 (m, 2H), 1.11-1.06 (m, 1H). | 428.1 | R-isomer: 100% ee; [α] = +88.226°; S-isomer: 97% ee; [α] = −96.320° |
| 166 | 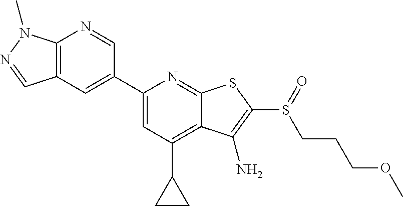 [SMILES: COCCCS(=O)c1sc2nc(-c3cnc4c(cnn4C)c3)cc(C3CC3)c2c1N] | 9.23 (d, J = 2.0 Hz, 1H), 8.73 (d, J = 2.0 Hz, 1H), 8.10 (s, 1H), 7.49 (d, J = 0.8 Hz, 1H), 5.60 (s, 2H), 4.22 (s, 3H), 3.59-3.53 (m, 2H), 3.46-3.39 (m, 1H), 3.38 (s, 3H), 3.33-3.24 (m, 1H), 2.61-2.53 (m, 1H), 2.13-2.05 (m, 2H), 1.26- 1.18 (m, 2H), 1.17-1.10 (m, 1H), 1.09-1.03 (m, 1H). | 442.1 | R-isomer: 100% ee; [α] = +97.440°; S-isomer: 98.3% ee; [α] = −105 808°; |
| 167 | 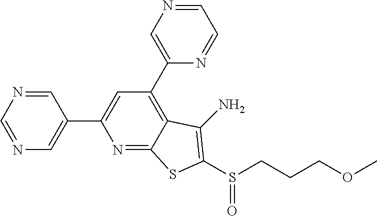 [SMILES: COCCCS(=O)c1sc2nc(-c3cncnc3)cc(-c3cnccn3)c2c1N] | CD₃OD 9.62 (s, 2H), 9.28 (s, 1H), 9.22 (d, J = 1.6 Hz, 1H), 8.87-8.84 (m, 2H), 8.24 (s, 1H), 3.54 (t, J = 6.0 Hz, 2H), 3.42-3.38 (m, 1H), 3.35 (s, 3H), 3.29-3.22 (m, 1H), 2.05-1.96 (m, 2H). | 427.1 | R-isomer: 100% ee; [α] = +138.624°; S-isomer: 100% ee; [α] = −130.813° |
| 168 | 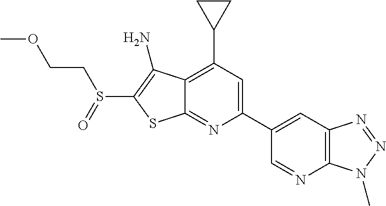 [SMILES: COCCS(=O)c1sc2nc(-c3cnc4c(c3)nnn4C)cc(C3CC3)c2c1N] | 9.43 (d, J = 1.6 Hz, 1H), 8.91 (d, J = 1.6 Hz, 1H), 7.53 (s, 1H), 5.58 (s, 2H), 4.42 (s, 3H), 3.94-3.88 (m, 1H), 3.76-3.71 (m, 1H), 3.67-3.62 (m, 1H), 3.43 (s, 3H), 3.35-3.29 (m, 1H), 2.61-2.54 (m, 1H), 1.27-1.22 (m, 2H), 1.16-1.12 (m, 1H), 1.09-1.04 (m, 1H). | 429.0 | R-isomer: 100% ee; [α] = +56.429°; S-isomer: 98% ee; [α] = −44.012° |
| 169 | 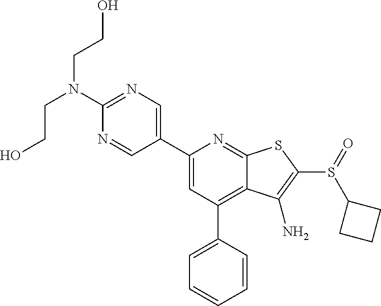 [SMILES: Nc1c(S(=O)C2CCC2)sc2nc(-c3cnc(N(CCO)CCO)nc3)cc(-c3ccccc3)c12] | 9.01 (s, 2H), 7.56-7.55 (m, 3H), 7.48 (s, 2H), 7.37 (s, 1H), 4.55 (s, 2H), 3.98- 3.95 (m, 4H), 3.92-3.90 (m, 4H), 3.78-3.73 (m, 1H), 2.83-2.76 (m, 1H), 2.39-2.34 (m, 2H), 2.25-2.22 (m, 1H), 2.10-2.06 (m, 2H). | 510.1 | R-isomer: 98% ee; [α] = +73.795. S-isomer: 96% ee; [α] = −86.782. |
| 170 | 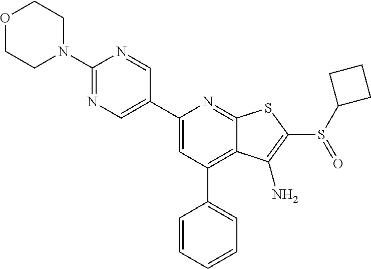 [SMILES: Nc1c(S(=O)C2CCC2)sc2nc(-c3cnc(N4CCOCC4)nc3)cc(-c3ccccc3)c12] | 9.05 (s, 2H), 7.57-7.56 (m, 3H), 7.50-7.49 (m, 2H), 7.40 (s, 1H), 4.56 (s, 2H), 4.00-3.93 (m, 5H), 3.83- 3.80 (m, 4H), 2.80-2.78 (m, 1H), 2.41-2.36 (m, 2H), 2.28-2.24 (m, 1H), 2.11- 2.07 (m, 2H). | 492.1 | |
| 171 | 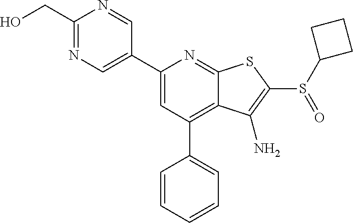 [SMILES: Nc1c(S(=O)C2CCC2)sc2nc(-c3cnc(CO)nc3)cc(-c3ccccc3)c12] | DMSO-d6 9.54 (s, 2H), 8.04 (s, 1H), 7.62 (s, 5H), 5.43 (t, J = 6.4 Hz, 1H), 4.96 (s, 2H), 4.69 (m, J = 6.0 Hz, 2H), 3.91- 3.83 (m, 1H), 2.68-2.63 (m, 1H), 2.18-2.15 (m, 3H), 2.01-1.90 (m, 2H). | 437.2 | |
| 172 | 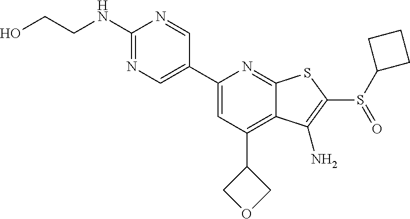 [SMILES: Nc1c(S(=O)C2CCC2)sc2nc(-c3cnc(NCCO)nc3)cc(C3COC3)c12] | DMSO-d6 9.10 (s, 2H), 7.81 (s, 1H), 7.58-7.55 (m, 1H), 5.58 (s, 2H), 5.24-5.22 (m, 1H), 5.01-4.95 (m, 3H), 4.93-4.71 (m, 2H), 3.83-3.81 (m, 1H), 3.57-3.54 (m, 2H), 3.47- 3.44 (m, 2H), 2.63-2.60 (m, 1H), 2.20-2.14 (m, 3H), 2.02-1.91 (m, 2H). | 446.1 | |
| 173 | 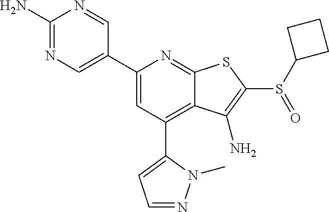 [SMILES: Cn1nccc1-c1cc(-c2cnc(N)nc2)nc2sc(S(=O)C3CCC3)c(N)c12] | 9.05 (s, 2H), 7.68 (d, J = 1.6 Hz, 1H), 7.48 (s, 1H), 6.49 (s, 1H), 5.33 (s, 2H), 4.52 (d, J = 37.6 Hz, 2H), 4.00-3.92 (m, 1H), 3.76 (s, 3H), 2.88-2.81 (m, 1H), 2.42-2.39 (m, 2H), 2.38- 2.37 (m, 1H), 2.13-2.09 (m, 2H). | 426.0 | R-isomer: 100% ee; [α] = +156.573°; S-isomer: 97% ee; [α] = −174.310°; |
| 174 | 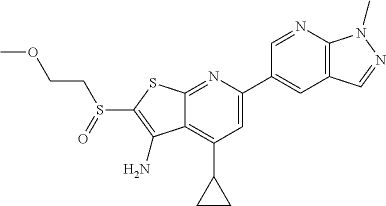 [SMILES: COCCS(=O)c1sc2nc(-c3cnc4c(cnn4C)c3)cc(C3CC3)c2c1N] | 9.23 (d, J = 2.0 Hz, 1H), 8.75 (d, J = 2.0 Hz, 1H), 8.11 (s, 1H), 7.51 (s, 1H), 5.56 (s, 1H), 4.22 (s, 3H), 3.91-3.89 (m, 1H), 3.74- 3.73 (m, 1H), 3.66-3.63 (m, 1H), 3.44 (s, 3H), 3.34- 3.29 (m, 1H), 2.57-2.53 (m, 1H), 1.23-1.21 (m, 2H), 1.12-1.10 (m, 1H), 1.08-1.07 (m, 1H). | 428.1 | R-isomer: 100% ee; [α] = +73.395. S-isomer: 100% ee; [α] = −59.486. |
| 175 | 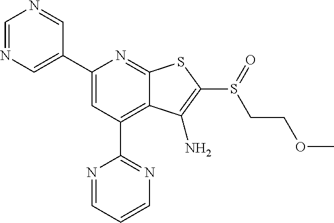 [SMILES: COCCS(=O)c1sc2nc(-c3cncnc3)cc(-c3ncccn3)c2c1N] | 9.56 (d, J = 4.0 Hz, 2H), 9.32 (s, 1H), 9.16 (d, J = 4.8 Hz, 2H), 8.53 (s, 1H), 7.77 (t, J = 4.8 Hz, 1H), 6.32 (s, 2H), 3.76-3.74 (m 1H), 3.64 (m, 1H), 3.44-3.41 (m, 1H), 3.40-3.26 (m, 4H). | 413.0 | R-isomer: 100% ee; [α] = +127.997. S-isomer: 100% ee; [α] = −231.162. |
| 176 | 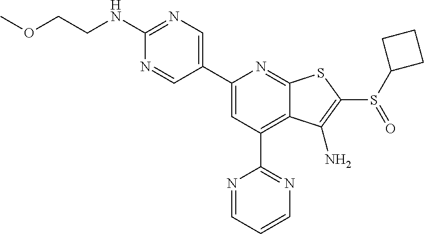 [SMILES: COCCNc1ncc(-c2cc(-c3ncccn3)c3c(N)c(S(=O)C4CCC4)sc3n2)cn1] | 9.08 (s, 2H), 8.98 (d, J = 5.2 Hz, 2H), 8.27 (s, 1H), 7.46 (t, J = 4.8 Hz, 1H), 6.32 (s, 2H), 5.73 (t, J = 5.6 Hz, 1H), 4.00-3.96 (m 1H), 3.74-3.70 (m, 2H), 3.63- 3.60 (m, 2H), 3.41 (s, 3H), 2.86-2.80 (m, 1H), 2.40- 2.35 (m, 2H), 2.20-2.15 (m, 1H), 2.10-2.06 (m, 2H). | 379.0 | |
| 177 | 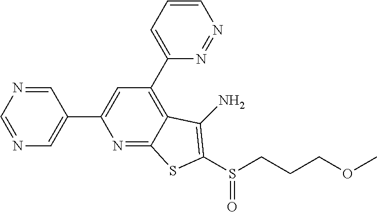 [SMILES: COCCCS(=O)c1sc2nc(-c3cncnc3)cc(-c3cccnn3)c2c1N] | DMSO-d6 9.63 (s, 2H), 9.48-7.46 (m, 1H), 9.32 (s, 1H), 8.39- 8.36 (m, 2H), 8.10-8.06 (m, 1H), 5.87 (s, 2H), 3.43 (t, J = 6.4 Hz, 2H), 3.22 (s, 3H), 3.21-3.14 (m, 1H), 3.13-3.04 (m, 1H), 1.88- 1.81 (m, 2H). | 427.2 | |
| 178 | 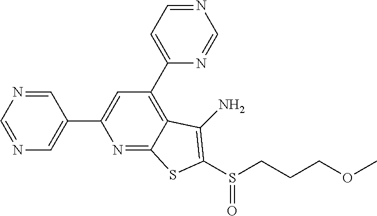 [SMILES: COCCCS(=O)c1sc2nc(-c3cncnc3)cc(-c3ccncn3)c2c1N] | 9.47 (s, 2H), 9.44 (d, J = 1.2 Hz, 1H), 9.34 (s, 1H), 9.06 (d, J = 4.8 Hz, 1H), 7.80 (dd, J1 = 4.8 Hz, J2 = 1.2 Hz, 1H), 7.77 (s, 1H), 5.68 (s, 2H), 3.54 (t, J = 6.0 Hz, 2H), 3.37 (m, 1H), 3.36 (s, 3H), 3.31-3.23 (m, 1H), 2.11-2.03 (m, 2H); | 427.1 | R-isomer: 98.6% ee; [α] = +124.899°; S-isomer: 100% ee; [α] = −141.197°; |
| 179 | 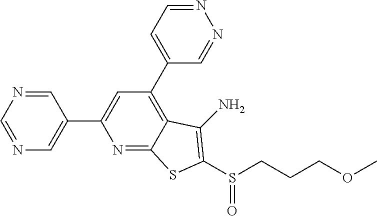 [SMILES: COCCCS(=O)c1sc2nc(-c3cncnc3)cc(-c3ccnnc3)c2c1N] | DMSO-d6 9.60 (s, 2H), 9.51-9.50 (m, 1H), 9.44-9.43 (m, 1H), 9.31 (s, 1H), 8.22 (s, 1H), 7.96-7.94 (m, 1H), 5.16 (s, 2H), 3.45 (t, J = 6.0 Hz, 2H), 3.24 (s, 3H), 3.21-3.16 (m, 1H), 3.13-3.06 (m, 1H), 1.89 (s, 2H). | 427.0 | |
| 180 | 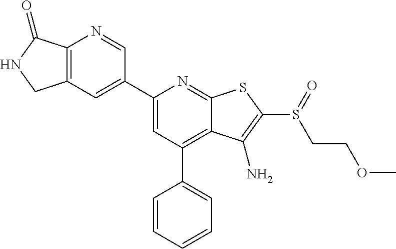 [SMILES: COCCS(=O)c1sc2nc(-c3cnc4c(c3)CNC4=O)cc(-c3ccccc3)c2c1N] | 9.44 (s, 1H), 8.73 (s, 1H), 7.71 (s, 1H), 7.62-7.48 (m, 5H), 6.74 (s, 1H), 4.62 (s, 2H), 4.60 (s, 2H), 3.95- 3.83 (m, 1H), 3.74-3.69 (m, 1H), 3.65-3.57 (m, 1H), 3.40 (s, 3H), 3.29 (m, 1H). | 465.1 | R-isomer: 98% ee; [α] = +49.923°; S-isomer: 98% ee; [α] = −43.697° |
| 181 | 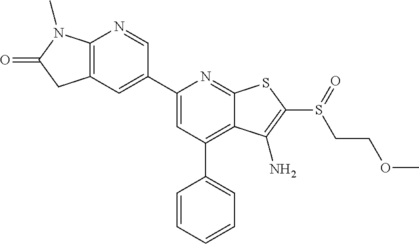 [SMILES: COCCS(=O)c1sc2nc(-c3cnc4c(c3)CC(=O)N4C)cc(-c3ccccc3)c2c1N] | 8.86 (d, J = 1.6 Hz, 1H), 8.38 (d, J = 0.8 Hz, 1H), 7.61-7.57 (m, 3H), 7.55 (s, 1H), 7.52 (s, 2H), 4.60 (s, 2H), 3.91-3.85 (m, 1H), 3.74-3.68 (m, 1H), 3.65 (s, 2H), 3.64-3.58 (m, 1H), 3.41 (s, 3H), 3.38 (s, 3H), 3.32-3.25 (m, 1H). | 479.2 | |
| 182 | 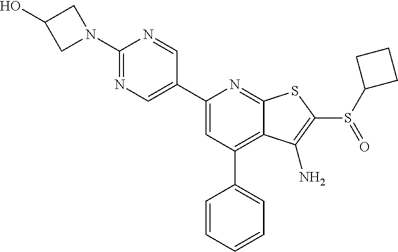 [SMILES: Nc1c(S(=O)C2CCC2)sc2nc(-c3cnc(N4CC(O)C4)nc3)cc(-c3ccccc3)c12] | 8.99 (s, 2H), 7.56-7.49 (m, 5H), 7.36 (s, 1H), 4.85- 4.81 (m, 1H), 4.55-4.48 (m, 4H), 4.13-4.09 (m, 2H), 3.98-3.94 (m, 1H), 2.81- 2.75 (m, 1H), 2.47-2.45 (m, 1H), 2.41-2.31 (m, 2H), 2.28-2.18 (m, 1H), 2.09-2.05 (m, 2H). 478.1 | | |
| 183 | 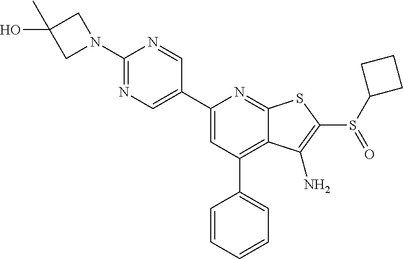 [SMILES: CC1(O)CN(c2ncc(-c3cc(-c4ccccc4)c4c(N)c(S(=O)C5CCC5)sc4n3)cn2)C1] | DMSO-d6 9.12 (s, 2H), 7.76 (s, 1H), 7.64-7.51 (m, 5H), 5.70 (s, 1H), 4.90 (s, 2H), 4.06- 3.93 (m, 4H), 3.84 (m, 1H), 2.64 (m, 1H), 2.17 (m, 3H), 2.09-1.97 (m, 1H), 1.97- 1.84 (m, 1H), 1.45 (s, 3H). | 492.2 | |
| 184 | 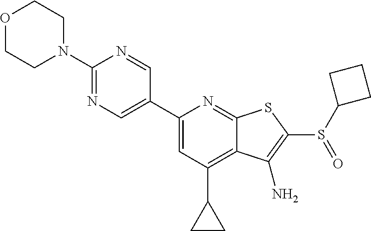 [SMILES: Nc1c(S(=O)C2CCC2)sc2nc(-c3cnc(N4CCOCC4)nc3)cc(C3CC3)c12] | 8.99 (s, 2H), 7.28 (s, 1H), 5.53 (s, 2H), 4.01-3.97 (m, 1H), 3.93-3.90 (m, 4H), 3.81-3.79 (m, 4H), 2.83-2.80 (m 1H), 2.51-2.50 (m, 1H), 2.43-2.40 (m, 2H), 2.35- 2.28 (m, 1H), 2.12-2.08 (m, 2H), 1.18-1.16 (m, 2H), 1.05-1.00 (m, 2H). | 456.1 | |
| 185 | 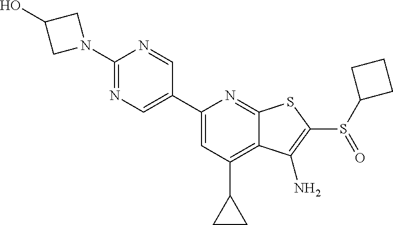 [SMILES: Nc1c(S(=O)C2CCC2)sc2nc(-c3cnc(N4CC(O)C4)nc3)cc(C3CC3)c12] | 8.94 (s, 2H), 7.27 (s, 1H), 5.53 (s, 2H), 4.84-4.83 (m, 1H), 4.52-4.47 (m, 2H), 4.12-4.09 (m, 2H), 3.98-3.96 (m 1H), 2.81-2.80 (m, 1H), 2.52-2.50 (m, 1H), 2.42- 2.31 (m, 3H), 2.38-2.26 (m, 1H), 2.11-2.08 (m, 2H), 1.18-1.16 (m, 2H), 1.08- 1.00 (m, 2H). | 442.0 | |
| 186 | 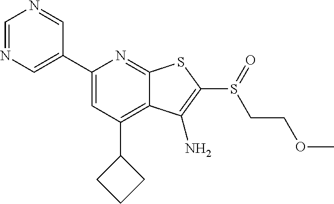 [SMILES: COCCS(=O)c1sc2nc(-c3cncnc3)cc(C3CCC3)c2c1N] | 9.42 (s, 2H), 9.30 (s, 1H), 7.66 (s, 1H), 5.15 (s, 2H), 4.28-4.20 (m, 1H), 3.92- 3.87 (m, 1H), 3.74-3.68 (m, 1H), 3.65-3.59 (m, 1H), 3.42 (s, 3H), 3.32-3.27 (m, 1H), 2.57-2.50 (m, 2H), 2.48-2.39 (m, 2H), 2.27-2.14 (m, 1H), 2.09-1.99 (m, 1H). | 389.1 | R-isomer: 99% ee; [α] = +55.025°; S-isomer: 100% ee; [α] = −49.891°; |
| 187 | 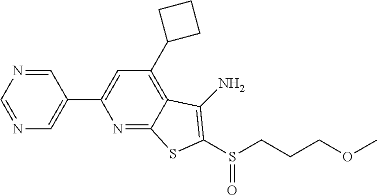 [SMILES: COCCCS(=O)c1sc2nc(-c3cncnc3)cc(C3CCC3)c2c1N] | 9.40 (s, 2H), 9.28 (s, 1H), 7.64 (s, 1H), 5.19 (s, 2H), 4.28-4.19 (m, 1H), 3.53 (t, J = 6.4 Hz, 2H), 3.41-3.37 (m, 1H), 3.35 (s, 3H), 3.28- 3.21 (m, 1H), 2.55-2.49 (m, 2H), 2.47-2.35 (m, 2H), 2.24-2.13 (m, 1H), 2.09- 1.98 (m, 3H). | 403.1 | |
| 188 | 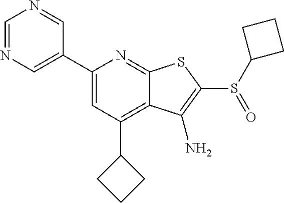 [SMILES: Nc1c(S(=O)C2CCC2)sc2nc(-c3cncnc3)cc(C3CCC3)c12] | DMSO-d6 9.55 (s, 2H), 9.29 (s, 1H), 7.98 (s, 1H), 5.77 (s, 2H), 4.50-4.41 (m, 1H), 3.92- 3.84 (m, 1H), 2.67-2.60 (m, 2H), 2.46-2.34 (m, 4H), 2.21- 2.13 (m, 2H), 2.10-1.99 (m, 2H), 1.97-1.82 (m, 2H). | 385.3 | |
| 189 | 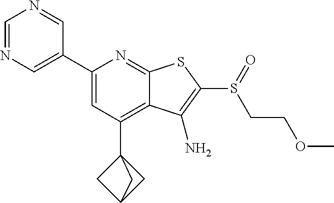 [SMILES: COCCS(=O)c1sc2nc(-c3cncnc3)cc(C34CC(C3)C4)c2c1N] | 9.42 (s, 2H), 9.31 (s, 1H), 7.48 (s, 1H), 5.38 (s, 2H), 3.96-3.89 (m, 1H), 3.77- 3.71 (m, 1H), 3.69-3.63 (m, 1H), 3.45 (s, 3H), 3.37-3.29 (m, 1H), 2.79 (s, 1H), 2.47 (s, 6H). | 401.0 | |
| 190 | 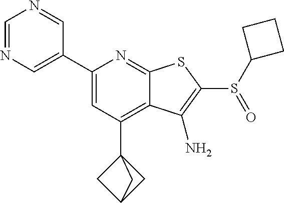 [SMILES: Nc1c(S(=O)C2CCC2)sc2nc(-c3cncnc3)cc(C34CC(C3)C4)c12] | 9.38 (s, 2H), 9.28 (s, 1H), 7.43 (s, 1H), 5.35 (s, 2H), 4.03-3.95 (m, 1H), 2.88- 2.77 (m, 1H), 2.76 (s, 1H), 2.47-2.41 (m, 7H), 2.39- 2.33 (m, 1H), 2.31-2.22 (m, 1H), 2.14-2.06 (m, 2H). | 397.1 | |
| 191 | 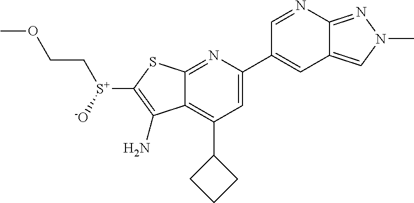 | 9.36 (d, J = 2.4 Hz, 1H), 8.70 (d, J = 2.4 Hz, 1H), 7.99 (s, 1H), 7.66 (s, 1H), 5.09 (s, 2H), 4.30 (s, 3H), 4.25-4.16 (m, 1H), 3.91- 3.85 (m, 1H), 3.71-3.66 (m, 1H), 3.64-3.58 (m, 1H), 3.41 (s, 3H), 3.30-3.23 (m, 1H), 2.58-2.43 (m, 3H), 2.41-2.32 (m, 1H), 2.23-2.12 (m, 1H), 2.05-1.96 (m, 1H). | 427.1 | R-isomer: 99% ee; [α] = +119.373°; S-isomer: 99% ee; [α] = −131.920°; |
| 192 | 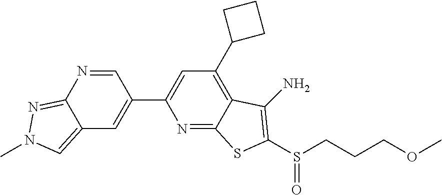 | DMSO-d6 9.42 (d, J = 2.4 Hz, 1H), 9.03 (d, J = 2.0 Hz, 1H), 8.56 (s, 1H), 7.93 (s, 1H), 5.76 (s, 2H), 4.48-4.38 (m, 1H), 4.26 (s, 3H), 3.45 (t, J = 6.4 Hz, 2H), 3.24 (s, 3H), 3.20-3.12 (m, 1H), 3.09-3.00 (m, 1H), 2.45-2.32 (m, 4H), 2.15-2.02 (m, 1H), 1.91-1.82 (m, 3H). | 456.2 | R-isomer: 100% ee; [α] = +23.834°; S-isomer: 97% ee; [α] = −24.371°; |
| 193 | 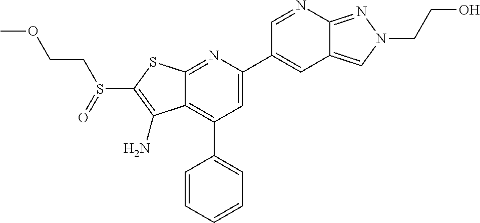 [SMILES: COCCS(=O)c1sc2nc(-c3cnc4nn(CCO)cc4c3)cc(-c3ccccc3)c2c1N] | 9.29 (d, J = 2.0 Hz, 1H), 8.72 (d, J = 2.4 Hz, 1H), 8.10 (s, 1H), 7.64-7.54 (m, 5H), 7.53-7.45 (m, 1H), 4.65-4.57 (m, 4H), 4.37- 4.21 (m, 2H), 3.97 (s, 1H), 3.90-3.83 (m, 1H), 3.73-3.67 (m, 1H), 3.62-3.55 (m, 1H), 3.41 (s, 3H), 3.31-3.23 (m, 1H). | 494.1 | R-isomer: 97.1% ee; [α] = +48.528°; S-isomer: 97% ee; [α] = −32.462°; |
| 194 | 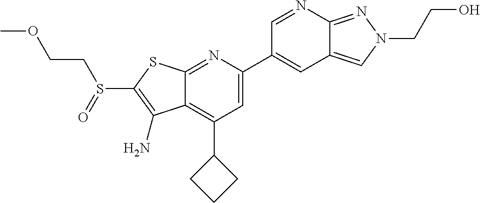 [SMILES: COCCS(=O)c1sc2nc(-c3cnc4nn(CCO)cc4c3)cc(C3CCC3)c2c1N] | 8.92 (d, J = 2.0 Hz, 1H), 8.36 (d, J = 2.0 Hz, 1H), 8.09 (s, 1H), 7.36 (s, 1H), 5.35-5.32 (m, 1H), 5.11 (s, 2H), 4.59-4.57 (m, 2H), 4.43 (m, 2H), 4.24-4.16 (m, 1H), 3.90-3.85 (m, 1H), 3.68- 3.63 (m, 1H), 3.65-3.55 (m, 1H), 3.41 (s, 3H), 3.25- 3.18 (m, 1H), 2.57-2.52 (m, 2H), 2.44-2.35 (m, 1H), 2.16-2.10 (m, 2H), 1.94- 1.87 (m, 1H). | 472.1 | R-isomer: 100% ee; [α] = +46.840°; S-isomer: 99% ee; [α] = −44.093°; |
| 195 | 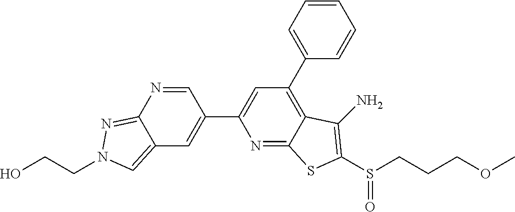 [SMILES: COCCCS(=O)c1sc2nc(-c3cnc4nn(CCO)cc4c3)cc(-c3ccccc3)c2c1N] | 9.33 (d, J = 2.4 Hz, 1H), 8.77 (d, J = 2.4 Hz, 1H), 8.10 (s, 1H), 7.60 (s, 2H), 7.59-7.55 (m, 3H), 7.53- 7.45 (m, 1H), 4.67 (s, 2H), 4.62-4.58 (m, 2H), 4.35- 4.18 (m, 2H), 3.73-3.66 (m, 1H), 3.55-3.49 (m, 2H), 3.39-3.35 (m, 1H), 3.34 (s, 3H), 3.26-3.17 (m, 1H), 2.07-1.98 (m, 2H). | 508.2 | |
| 196 | 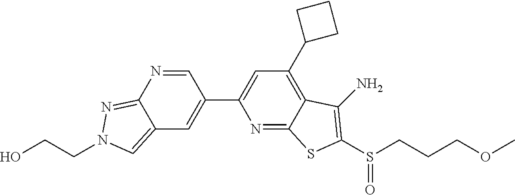 [SMILES: COCCCS(=O)c1sc2nc(-c3cnc4nn(CCO)cc4c3)cc(C3CCC3)c2c1N] | 9.21 (s, 1H), 8.61 (s, 1H), 8.10 (s, 1H), 7.57 (s, 1H), 5.15 (s, 2H), 4.60 (m, 2H), 4.33 (s, 2H), 4.27-4.18 (m, 1H), 4.13-4.05 (m, 1H), 3.53 (t, J = 5.6 Hz, 2H), 3.42- 3.38 (m, 1H), 3.36 (s, 1H), 3.27-3.18 (m, 1H), 2.60- 2.42 (m, 3H), 2.36-2.27 (m, 1H), 2.24-2.13 (m, 1H), 2.09-1.95 (m, 3H). | 486.1 | |
| 197 | 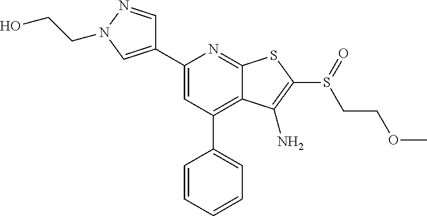 [SMILES: COCCS(=O)c1sc2nc(-c3cnn(CCO)c3)cc(-c3ccccc3)c2c1N] | 8.13 (s, 1H), 8.05 (s, 1H), 7.58-7.52 (m, 3H), 7.48- 7.45 (m, 2H), 7.29 (s, 1H), 4.53 (s, 2H), 4.33 (t, J = 3.2 Hz, 2H), 4.09-4.05 (m, 2H), 3.88-3.82 (m, 1H), 3.71-3.65 (m, 1H), 3.62- 3.55 (m, 1H), 3.39 (s, 3H), 3.29-3.21 (m, 1H), 2.95- 2.89 (m, 1H) | 443.2 | |
| 198 | 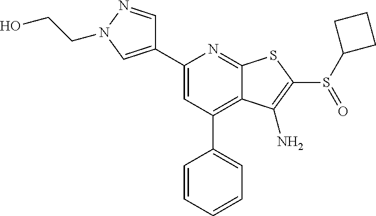 [SMILES: Nc1c(S(=O)C2CCC2)sc2nc(-c3cnn(CCO)c3)cc(-c3ccccc3)c12] | MeOD 8.35 (s, 1H), 8.15 (s, 1H), 7.61-7.56 (m, 3H), 7.54- 7.50 (m, 2H), 7.49 (s, 1H), 4.29 (t, J = 5.2 Hz, 2H), 3.99-3.89 (m, 3H), 2.81- 2.70 (m, 1H), 2.44-2.32 (m, 1H), 2.32-2.22 (m, 2H), 2.17-2.00 (m, 2H). | 439.2 | |
| 199 | 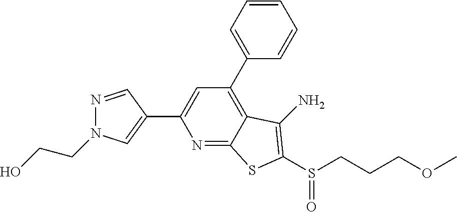 [SMILES: COCCCS(=O)c1sc2nc(-c3cnn(CCO)c3)cc(-c3ccccc3)c2c1N] | 8.13 (s, 1H), 8.05 (s, 1H), 7.57-7.52 (m, 3H), 7.48 (s, 2H), 7.28 (s, 1H), 4.55 (s, 2H), 4.32 (m, 2H), 4.09- 4.03 (m, 2H), 3.52-3.49 (m, 2H), 3.39-3.45 (m, 1H), 3.34 (s, 3H), 3.26-3.16 (m, 1H), 2.92 (t, J = 5.6 Hz, 1H), 2.06-1.98 (m, 2H). | 457.0 | |
| 200 | 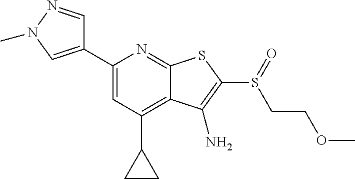 | 8.02 (s, 1H), 8.00 (s, 1H), 7.18 (s, 1H), 5.51 (s, 2H), 3.99 (s, 3H), 3.93-3.87 (m, 1H), 3.75-3.69 (m, 1H), 3.66-3.59 (m, 1H), 3.43 (s, 3H), 3.33-3.26 (m, 1H), 2.52-2.43 (m, 1H), 1.20- 1.15 (m, 2H), 1.06-1.02 (m, 2H). | 377.0 | R-isomer: 100% ee; [α] = +60.777°; S-isomer: 99% ee; [α] = −68.241°; |
| 201 | 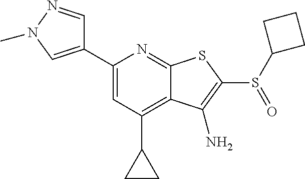 [SMILES: Cn1cc(-c2cc(C3CC3)c3c(N)c(S(=O)C4CCC4)sc3n2)cn1] | 8.00 (d, J = 5.6 Hz, 2H), 7.16 (s, 1H), 5.51 (s, 2H), 4.03-3.95 (m, 4H), 2.84- 2.80 (m, 1H), 2.49-2.41 (m 3H), 2.40-2.38 (m, 1H), 2.12-2.08 (m, 2H), 1.17-1.15 (m, 2H), 1.06-1.01 (m, 2H). | 373.1 | |
| 202 | 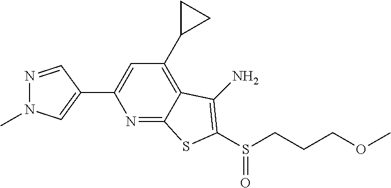 | 8.00 (s, 1H), 7.98 (s, 1H), 7.15 (s, 1H), 5.52 (s, 2H), 3.98 (s, 3H), 3.53 (m, 2H), 3.39-3.35 (m, 4H), 3.28- 3.21 (m, 1H), 2.49-2.45 (m, 1H), 2.08-2.01 (m, 2H), 1.16-1.14 (m, 2H), 1.05- 1.00 (m, 2H). | 391.0 | |
| 203 | 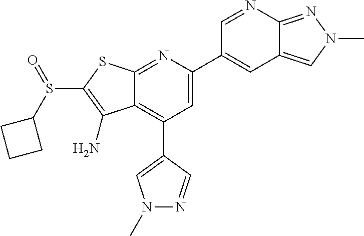 [SMILES: Cn1cc(-c2cc(-c3cnc4nn(C)cc4c3)nc3sc(S(=O)C4CCC4)c(N)c23)cn1] | DMSO-d6 8.70 (d, J = 1.6 Hz, 1H), 8.57 (s, 1H), 8.47 (s, 1H), 8.38 (d, J = 2.0 Hz, 1H), 8.14 (s, 1H), 7.66 (s, 1H), 4.92 (s, 2H), 4.27 (s, 3H), 3.89-3.82 (m, 4H), 2.66- 2.63 (m, 1H), 2.19-2.14 (m, 3H), 2.03-2.00 (m, 1H), 1.98-1.94 (m, 1H). | 464.1 | |
| 204 | 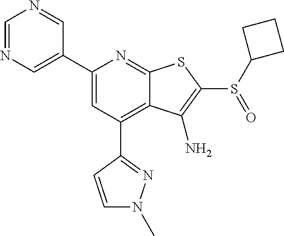 [SMILES: Cn1ccc(-c2cc(-c3cncnc3)nc3sc(S(=O)C4CCC4)c(N)c23)n1] | 9.45 (s, 2H), 9.31 (s, 1H), 7.81 (s, 1H), 7.59 (d, J = 2.0 Hz, 1H), 6.76 (d, J = 2.4 Hz, 1H), 6.45 (s, 2H), 4.07 (s, 3H), 4.01 (t, J = 8.0 Hz, 1H), 2.89-2.80 (m, 1H), 2.47-2.34 (m, 2H), 2.31-2.21 (m, 1H), 2.15- 2.04 (m, 2H). | 411.0 | |
| 205 | 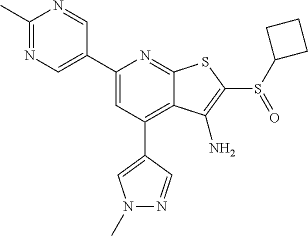 [SMILES: Cc1ncc(-c2cc(-c3cnn(C)c3)c3c(N)c(S(=O)C4CCC4)sc3n2)cn1] | 9.29 (s, 2H), 7.76 (s, 1H), 7.70 (s, 1H), 7.50 (s, 1H), 4.95 (s, 2H), 4.06 (s, 3H), 4.03-3.95 (m, 1H), 2.85- 2.80 (m, 4H), 2.44-2.36 (m, 2H), 2.31-2.26 (m, 1H), 2.14-2.10 (m, 2H). | 425.0 | |
| 206 | 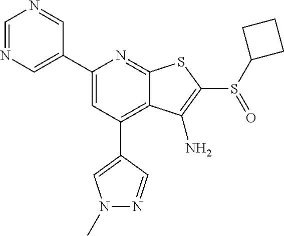 [SMILES: Cn1cc(-c2cc(-c3cncnc3)nc3sc(S(=O)C4CCC4)c(N)c23)cn1] | 9.40 (s, 2H), 9.29 (s, 1H), 7.76 (s, 1H), 7.71 (s, 1H), 7.53 (s, 1H), 4.97 (s, 2H), 4.06 (s, 3H), 4.01-3.95 (m, 1H), 2.87-2.80 (m, 1H), 2.42-2.37 (m, 2H), 2.28-2.26 (m, 1H), 2.13-2.08 (m, 2H). | 411.0 | |
| 207 | 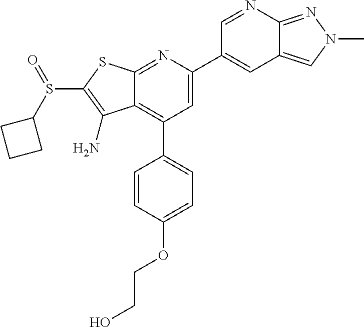 [SMILES: Cn1cc2cc(-c3cc(-c4ccc(OCCO)cc4)c4c(N)c(S(=O)C5CCC5)sc4n3)cnc2n1] | DMSO-d6 9.41 (d, J = 2.4 Hz, 1H), 9.03 (d, J = 2.4 Hz, 1H), 8.54 (s, 1H), 7.89 (s, 1H), 7.52 (d, J = 8.8 Hz, 2H), 7.16 (d, J = 8.8 Hz, 2H), 5.04 (s, 2H), 4.94 (t, J = 5.4 Hz, 1H), 4.23 (s, 3H), 4.11- 4.07 (m, 2H), 3.91-3.82 (m, 1H), 3.79-3.75 (m, 2H), 2.66-2.60 (m, 1H), 2.23- 2.14 (m, 3H), 2.06-1.97 (m, 1H), 1.96-1.88 (m, 1H). | 520.0 | R-isomer: 97% ee; [α] = +50.037°; S-isomer: 95% ee; [α] = −47.384°; |
| 208 | 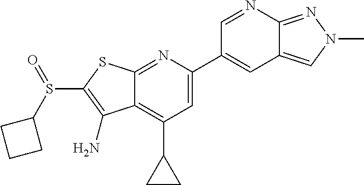 [SMILES: Cn1cc2cc(-c3cc(C4CC4)c4c(N)c(S(=O)C5CCC5)sc4n3)cnc2n1] | 9.32 (d, J = 2.4 Hz, 1H), 8.70 (d, J = 2.4 Hz, 1H), 8.00 (s, 1H), 7.47 (s, 1H), 5.54 (s, 2H), 4.29 (s, 3H), 4.03-3.95 (m, 1H), 2.84- 2.83 (m, 1H), 2.54-2.50 (m, 1H), 2.41-2.38 (m, 2H), 2.30-2.25 (m, 1H), 2.12-2.08 (m, 2H), 1.21-1.19 (m, 2H), 1.10-1.04 (m, 2H). | 424.2 | R-isomer: 99% ee; [α] = +86.00° S-isomer: 99% ee; [α] = −110.40° |
| 209 | 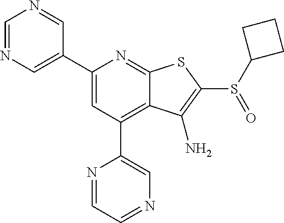 [SMILES: Nc1c(S(=O)C2CCC2)sc2nc(-c3cncnc3)cc(-c3cnccn3)c12] | DMSO-d6 9.62 (s, 2H), 9.31 (s, 1H), 9.28 (d, J = 1.2 Hz, 1H), 8.91-8.89 (m, 2H), 8.37 (s, 1H), 5.78 (s, 2H), 3.93- 3.89 (m, 1H), 2.68-2.60 (m, 1H), 2.30-2.21 (m, 1H), 2.21-2.07 (m, 2H), 2.07- 1.98 (m, 1H), 1.97-1.87 (m, 1H). | 409.0 | |
| 210 | 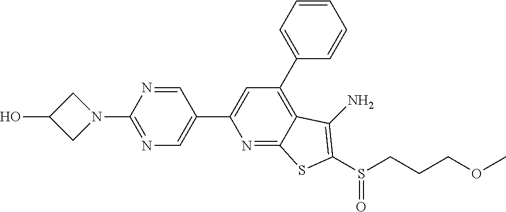 [SMILES: COCCCS(=O)c1sc2nc(-c3cnc(N4CC(O)C4)nc3)cc(-c3ccccc3)c2c1N] | 8.93 (s, 2H), 7.60-7.52 (m, 5H), 7.35 (s, 1H), 4.84- 4.76 (m, 1H), 4.61 (s, 2H), 4.53-4.45 (m, 2H), 4.11 (m, 2H), 3.54-3.48 (m, 2H), 3.40-3.35 (m, 1H), 3.34 (s, 3H), 3.27-3.17 (m, 2H), 2.06-1.97 (m, 2H). | 496.1 | |
| 211 | 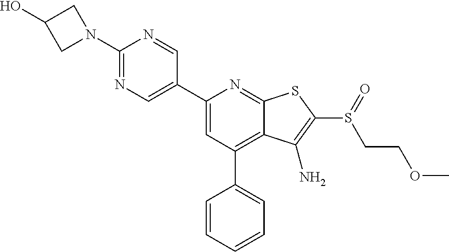 [SMILES: COCCS(=O)c1sc2nc(-c3cnc(N4CC(O)C4)nc3)cc(-c3ccccc3)c2c1N] | 8.98 (s, 2H), 7.60-7.56 (m, 3H), 7.54-7.50 (m, 2H), 7.38 (s, 1H), 4.87-4.79 (m, 1H), 4.58 (s, 2H),4.53- 4.48 (m, 2H), 4.12 (m, 2H), 3.89-3.83 (m, 1H), 3.72- 3.66 (m, 1H), 3.64-3.57 (m, 1H), 3.40 (s, 3H), 3.31- 3.24 (m, 1H), 2.72 (d, J = 6.4 Hz, 1H). | 482.1 | |
| 212 | 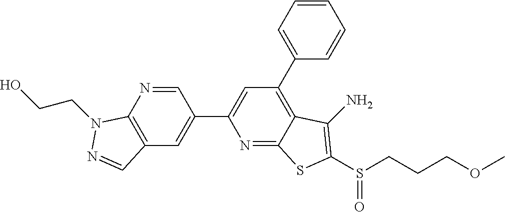 [SMILES: COCCCS(=O)c1sc2nc(-c3cnc4c(cnn4CCO)c3)cc(-c3ccccc3)c2c1N] | DMSO-d6 9.39-3.38 (d, 1H), 9.07- 9.06 (d, 1H), 8.26 (s, 1H), 7.98 (s, 1H), 7.61 (s, 5H), 4.95 (s, 2H), 4.87 (t, J = 5.6 Hz, 1H), 4.54 (t, J = 6.0 Hz, 2H), 3.91-3.85 (m, 2H), 3.42 (t, J = 2.4 Hz, 2H), 3.21 (s, 3H), 3.18-3.11 (m, 1H), 3.07-2.99 (m, 1H), 1.88-1.78 (m, 2H). | 508 | |
| 213 | 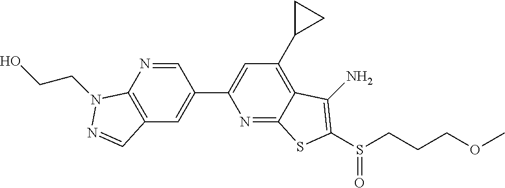 [SMILES: COCCCS(=O)c1sc2nc(-c3cnc4c(cnn4CCO)c3)cc(C3CC3)c2c1N] | 9.19 (d, J = 4.0 Hz, 1H), 8.77 (s, 1H), 8.11 (s, 1H), 7.48 (s, 1H), 5.59 (s, 2H), 4.75-4.73 (m, 2H), 4.22- 4.18 (m, 3H), 3.56-3.53 (m, 2H), 3.42-3.37 (m, 4H), 3.31-3.27 (m, 1H), 2.57- 2.55 (m, 1H), 2.11-2.06 (m, 2H), 1.23-1.21 (m, 2H), 1.13-1.06 (m, 2H). | 472.2 | |
| 214 | 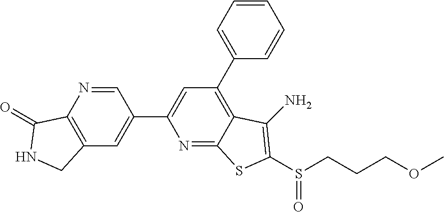 [SMILES: COCCCS(=O)c1sc2nc(-c3cnc4c(c3)CNC4=O)cc(-c3ccccc3)c2c1N] | 9.45 (s, 1H), 8.73 (d, J = 2.0 Hz, 1H), 7.72 (s, 1H), 7.63-7.50 (m, 5H), 6.90 (s, 1H), 4.68 (s, 2H), 4.62 (s, 2H), 3.56-3.51 (m, 2H), 3.43-3.37 (m, 1H), 3.36 (s, 3H), 3.30-3.22 (m, 1H), 2.11-2.02 (m, 2H). | 479.0 | |
| 215 | 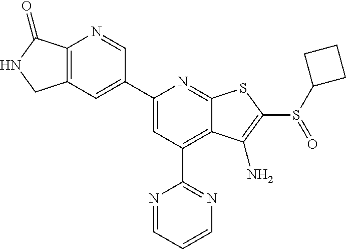 [SMILES: Nc1c(S(=O)C2CCC2)sc2nc(-c3cnc4c(c3)CNC4=O)cc(-c3ncccn3)c12] | DMSO-d6 9.48 (d, J = 1.6 Hz, 1H), 9.15 (d, J = 4.8 Hz, 2H), 9.08 (s, 1H), 8.81 (s, 1H), 8.54 (s, 1H), 7.78-7.74 (m, 1H), 6.34 (d, J = 5.6 Hz, 2H), 4.50 (s, 2H), 3.95-3.89 (m, 1H), 2.68-2.66 (m, 1H), 2.19-2.12 (m, 3H), 2.07- 1.92 (m, 2H). | 463.0 | |
| 216 | 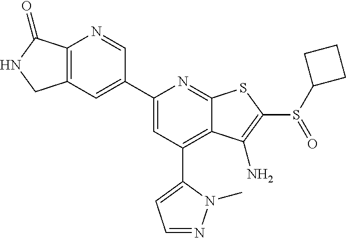 [SMILES: Cn1nccc1-c1cc(-c2cnc3c(c2)CNC3=O)nc2sc(S(=O)C3CCC3)c(N)c12] | DMSO-d6 9.53 (d, J = 2.0 Hz, 1H), 9.07 (s, 1H), 8.85 (d, J = 1.6 Hz, 1H), 8.22 (s, 1H), 7.70 (d, J = 1.6 Hz, 1H), 6.67 (s, 1H), 5.04 (s, 2H), 4.50 (s, 2H), 3.93-3.85 (m, 1H), 3.73 (s, 3H), 2.68-2.66 (m, 1H), 2.23-2.16 (m, 3H), 2.15-1.90 (m, 2H). | 465.0 | |
| 217 | 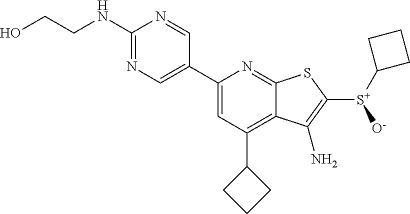 [SMILES: Nc1c([S@+]([O-])C2CCC2)sc2nc(-c3cnc(NCCO)nc3)cc(C3CCC3)c12] | 8.93 (s, 2H), 7.39 (s, 1H), 6.03-6.01 (m, 1H), 5.12 (s, 2H), 4.24-4.15 (m, 1H), 4.00- 3.90 (m, 4H), 3.72-3.68 (m, 2H), 2.88-2.78 (m, 1H), 2.53-2.47 (m, 2H), 2.45- 2.36 (m, 3H), 2.32-2.22 (m, 2H), 2.20-2.05 (m, 3H), 2.02-1.95 (m, 1H). | 444.4 | R-isomer: 99% ee; [α] = +158.691°; S-isomer: 100% ee; [α] = −151.194°; |
| 218 | 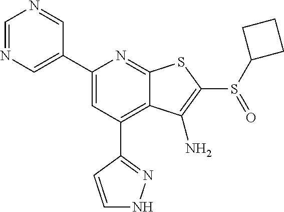 [SMILES: Nc1c(S(=O)C2CCC2)sc2nc(-c3cncnc3)cc(-c3cc[nH]n3)c12] | DMSO-d6 9.60 (s, 2H), 9.31 (s, 1H), 8.30 (s, 1H), 8.09 (d, J = 2.4 Hz, 1H), 7.25-7.23 (m, 3H), 3.91-3.87 (m, 1H), 2.68-2.67 (m, 1H), 2.23- 2.13 (m, 3H), 2.06-1.99 (m, 1H), 1.97-1.89 (m, 1H). | 397.1 | |
| 219 | 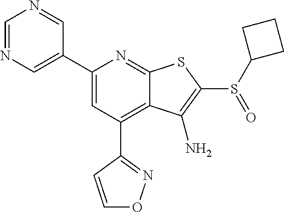 [SMILES: Nc1c(S(=O)C2CCC2)sc2nc(-c3cncnc3)cc(-c3ccon3)c12] | DMSO-d6 9.60 (s, 2H), 9.32-9.31 (m, 2H), 8.36 (s, 1H), 7.43 (s, 1H), 6.21 (s, 2H), 3.91 (t, J = 8.0 Hz, 1H), 2.67-2.62 (m, 1H), 2.27-2.07 (m, 3H), 2.05-1.98 (m, 1H), 1.96- 1.86 (m, 1H). | 398.1 | R-isomer: 99% ee; [α] = +222.308°; S-isomer: 100% ee; [α] = −277.961°; |
| 220 | 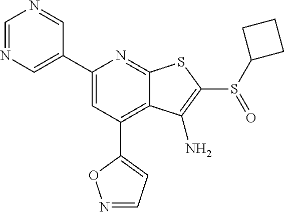 [SMILES: Nc1c(S(=O)C2CCC2)sc2nc(-c3cncnc3)cc(-c3ccno3)c12] | 9.44 (s, 2H), 9.32 (s, 1H), 8.52 (d, J = 1.6 Hz, 1H), 7.85 (s, 1H), 6.80 (d, J = 1.6 Hz, 1H), 5.15 (s, 2H), 4.02-3.94 (m, 1H), 2.87- 2.82 (m, 1H), 2.49-2.39 (m, 1H), 2.38-2.32 (m, 1H), 2.31- 2.23 (m, 1H), 2.15-2.06 (m, 2H). | 398.1 | |
| 221 | 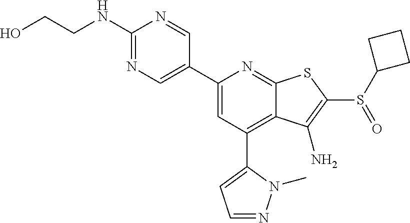 [SMILES: Cn1nccc1-c1cc(-c2cnc(NCCO)nc2)nc2sc(S(=O)C3CCC3)c(N)c12] | DMSO-d6 9.10 (s, 2H), 7.93 (s, 1H), 7.69 (s, 1H), 7.68-7.63 (m, 1H), 6.63 (s, 1H), 4.98 (s, 2H), 4.73 (t, J = 5.6 Hz, 1H), 3.88-3.84 (m, 1H), 3.70 (s, 3H), 3.56-3.53 (m, 2H), 3.46-3.43 (m, 2H), 2.65-2.64 (m, 1H), 2.20-2.16 (m, 3H), 2.15-1.90 (m, 2H). | 470.2 | R-isomer: 99% ee; [α] = +101.844°; S-isomer: 99% ee; [α] = −86.901°; |
| 222 | 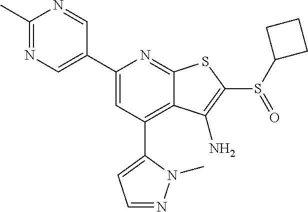 [SMILES: Cc1ncc(-c2cc(-c3ccnn3C)c3c(N)c(S(=O)C4CCC4)sc3n2)cn1] | DMSO-d6 9.47 (s, 2H), 8.18 (s, 1H), 7.70 (d, J = 2.0 Hz, 1H), 6.66 (s, 1H), 5.05 (s, 2H), 3.93-3.85 (m, 1H), 3.72 (s, 3H), 2.72 (s, 3H), 2.67- 2.66 (m, 1H), 2.23-2.16 (m, 3H), 2.15-1.90 (m, 2H). | 425.2 | |
| 223 | 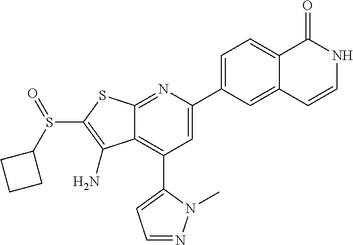 [SMILES: Cn1nccc1-c1cc(-c2ccc3c(=O)[nH]ccc3c2)nc2sc(S(=O)C3CCC3)c(N)c12] | 8.29-8.28 (m, 1H), 8.53 (d, J = 8.4 Hz, 1H), 8.33 (s, 1H), 8.21 (d, J = 8.4 Hz, 1H), 7.75 (s, 1H), 7.68 (s, 1H), 7.14 (t, J = 6.4 Hz, 1H), 6.65 (d, J = 7.2 Hz, 1H), 6.51 (s, 1H), 4.66-4.47 (m, 2H), 4.03-3.89 (m, 1H), 3.82-3.71 (s, 3H), 2.92- 2.76 (m, 1H), 2.50-2.18 (m, 3H), 2.15-1.99 (m, 2H). | 476.3 | |
| 224 | 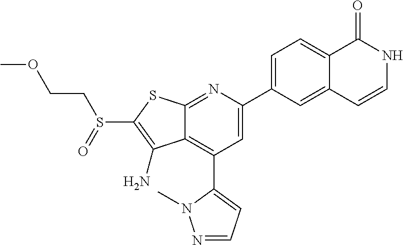 [SMILES: COCCS(=O)c1sc2nc(-c3ccc4c(=O)[nH]ccc4c3)cc(-c3ccnn3C)c2c1N] | DMSO-d6 11.35-11.33 (m, 1H), 8.59 (d, J = 1.6 Hz, 1H), 8.38- 8.36 (m, 1H), 8.32-8.30 (m, 1H), 8.19 (s, 1H), 7.72 (d, J = 2 Hz, 1H), 7.26- 7.23 (m, 1H), 6.71-6.68 (m, 2H), 5.06 (s, 2H), 3.79- 3.76 (m, 1H), 3.74 (s, 3H), 3.68-3.63 (m, 1H), 3.43- 3.38 (m, 1H), 3.29 (s, 3H), 3.27-3.22 (m, 1H) | 480.1 | |
| 225 | 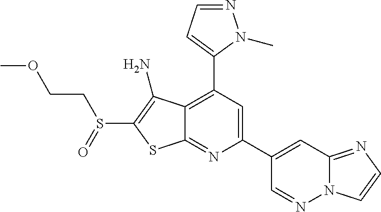 [SMILES: COCCS(=O)c1sc2nc(-c3cnn4ccnc4c3)cc(-c3ccnn3C)c2c1N] | 9.28 (d, J = 2.0 Hz, 1H), 8.56 (d, J = 2.0 Hz, 1H), 8.08 (s, 1H), 7.90 (d, J = 1.2 Hz, 1H), 7.73-7.68 (m, 2H), 6.52 (s, 1H), 4.59-4.53 (m, 2H), 3.89 (m, 1H), 3.79 (s, 3H), 3.76-3.69 (m, 1H), 3.65-3.58 (m, 1H), 3.41 (s, 3H), 3.29 (m, 1H) | 454.1 | |
| 226 | 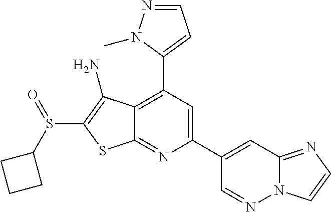 [SMILES: Cn1nccc1-c1cc(-c2cnn3ccnc3c2)nc2sc(S(=O)C3CCC3)c(N)c12] | 9.27 (d, J = 2.0 Hz, 1H), 8.55 (d, J = 2.0 Hz, 1H), 8.08 (s, 1H), 7.89 (d, J = 0.8 Hz, 1H), 7.69 (d, J = 1.6 Hz, 2H), 7.68 (s, 1H), 6.51 (s, 1H), 4.56 (d, J = 38.4 Hz, 2H), 3.98-3.94 (m, 1H), 3.77 (s, 3H), 2.90-2.77 (m, 1H), 2.47-2.22 (m, 3H), 2.17-2.03 (m, 2H). | 450.1 | |
| 227 | 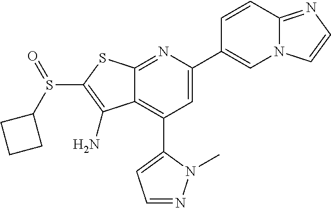 [SMILES: Cn1nccc1-c1cc(-c2ccc3nccn3c2)nc2sc(S(=O)C3CCC3)c(N)c12] | DMSO-d6 9.88 (d, J = 2.8 Hz, 1H), 9.36 (d, J = 2.4 Hz, 1H), 8.15 (s, 1H), 8.01 (s, 1H), 7.80 (s, 1H), 7.70 (d, J = 1.6 Hz, 1H), 6.66 (s, 1H), 5.05 (s, 2H), 3.90-3.84 (m, 1H), 3.74 (s, 3H), 2.67-2.61 (m, 1H), 2.26-2.12 (m, 3H), 2.09-1.88 (m, 2H) | 450.1 | R-isomer: 98% ee; [α] = +64.999°; S-isomer: 99% ee; [α] = −223.632° |
| 228 | 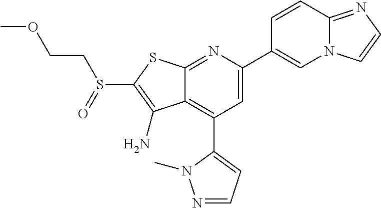 [SMILES: COCCS(=O)c1sc2nc(-c3ccc4nccn4c3)cc(-c3ccnn3C)c2c1N] | DMSO-d6 9.91 (d, J = 2.4 Hz, 1H), 9.38 (d, J = 2.8 Hz, 1H), 8.18 (s, 1H), 8.03 (d, J = 1.2 Hz, 1H), 7.82 (d, J = 1.6 Hz, 1H), 7.72 (d, J = 2.0 Hz, 1H), 6.67 (d, J = 1.2 Hz, 1H), 5.09 (s, 2H), 3.77-3.73 (m, 4H), 3.66 (m, 1H), 3.39-3.37 (m, 1H), 3.29-3.24 (m, 4H). | 454.2 | |
| 229 | 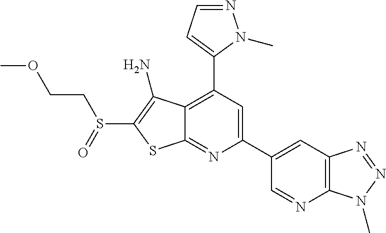 [SMILES: COCCS(=O)c1sc2nc(-c3cnc4c(c3)nnn4C)cc(-c3ccnn3C)c2c1N] | DMSO-d6 9.64 (d, J = 1.6 Hz, 1H), 9.35 (d, J = 1.6 Hz, 1H), 8.30 (s, 1H), 7.71 (d, J = 1.6 Hz, 1H), 6.67 (s, 1H), 5.07 (s, 2H), 4.36 (s, 3H), 3.78-3.72 (m, 4H), 3.67- 3.62 (m, 1H), 3.41-3.37 (m, 1H), 3.28 (s, 3H), 3.26- 3.21 (m, 1H). | 469.3 | |
| 230 | 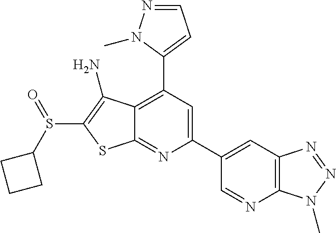 [SMILES: Cn1nccc1-c1cc(-c2cnc3c(c2)nnn3C)nc2sc(S(=O)C3CCC3)c(N)c12] | DMSO-d6 9.62 (s, 1H), 9.33 (d, J = 1.6 Hz, 1H), 8.27 (s, 1H), 7.70 (d, J = 2.0 Hz, 1H), 6.67 (s, 1H), 5.04 (s, 2H), 4.36 (s, 3H), 3.93-3.85 (m, 1H), 3.74 (s, 3H), 2.70- 2.65 (m, 1H), 2.25-2.12 (m, 3H), 2.08-1.99 (m, 1H), 1.97-1.88 (m, 1H). | 465.2 | R-isomer: 97% ee; [α] = +146.313°; S-isomer: 100% ee; [α] = −145.920°; |
| 231 | 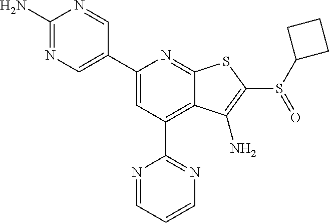 [SMILES: Nc1ncc(-c2cc(-c3ncccn3)c3c(N)c(S(=O)C4CCC4)sc3n2)cn1] | DMSO-d6 9.12 (d, J = 5.2 Hz, 2H), 9.02 (s, 2H), 8.23 (s, 1H), 7.73 (t, J = 4.8 Hz, 1H), 7.18 (s, 2H), 6.21 (s, 2H), 3.88 (t, J = 6.0 Hz, 1H), 2.66- 2.61 (m, 1H), 2.21-2.16 (m, 3H), 2.02-1.90 (m, 2H). | 424.2 | |
| 232 | 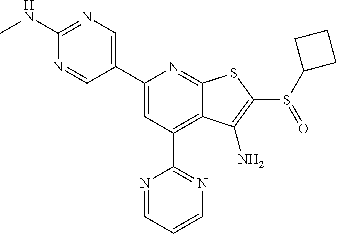 [SMILES: CNc1ncc(-c2cc(-c3ncccn3)c3c(N)c(S(=O)C4CCC4)sc3n2)cn1] | 9.09 (s, 2H), 8.98 (d, J = 4.8 Hz, 2H), 8.27 (s, 1H), 7.46 (t, J = 4.8 Hz, 1H), 6.32 (s, 2H), 5.40-5.38 (m, 1H), 3.98 (t, J = 8.0 Hz, 1H), 3.11-3.09 (m, 3H), 2.85- 2.80 (m, 1H), 2.39-2.34 (m, 2H), 2.27-2.25 (m, 1H), 2.10-2.05 (m, 2H). | 438.2 | |
| 233 | 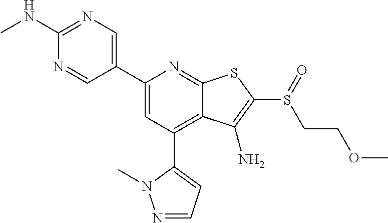 | 9.03 (s, 2H), 7.67 (d, J = 2.0 Hz, 1H), 7.45 (s, 1H), 6.49-6.48 (m, 1H), 5.43 (t, J = 4.8 Hz, 1H), 4.52-4.45 (m, 2H), 3.83-3.81 (m, 1H), 3.76 (s, 3H), 3.75-3.71 (m, 1H), 3.61-3.58 (m, 1H), 3.40 (s, 3H), 3.29-3.23 (m, 1H), 3.11-3.10 (m, 3H). | 444.2 | R-isomer: 98% ee; [α] = +95.687. S-isomer: 98% ee; [α] = −111.509. |
| 234 | 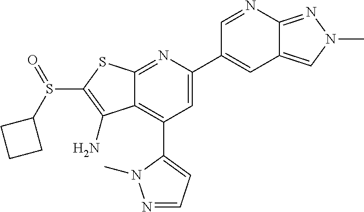 [SMILES: Cn1cc2cc(-c3cc(-c4ccnn4C)c4c(N)c(S(=O)C5CCC5)sc4n3)cnc2n1] | DMSO-d6 9.44 (d, J = 1.6 Hz, 1H), 9.06 (d, J = 1.6 Hz, 1H), 8.57 (s, 1H), 8.12 (s, 1H), 7.69 (s, 1H), 6.65 (s, 1H), 5.01 (s, 2H), 4.24 (s, 3H), 3.89-3.83 (m, 1H), 3.72 (s, 3H), 2.67-2.60 (m, 1H), 2.24- 2.15 (m, 3H), 2.07-1.99 (m, 1H), 1.96-1.88 (m, 1H). | 464.3 | |
| 235 | 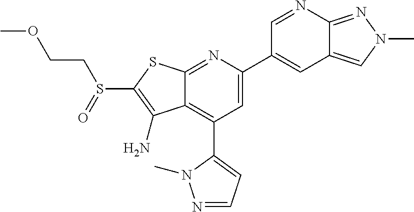 | DMSO-d6 9.45 (d, J = 2.0 Hz, 1H), 9.08 (d, J = 2.0 Hz, 1H), 8.57 (s, 1H), 8.14 (s, 1H), 7.70 (s, 1H), 6.66 (s, 1H), 5.04 (s, 2H), 4.24 (s, 3H), 3.77-3.72 (m, 4H), 3.65- 3.62 (m, 1H), 3.41-3.36 (m, 1H), 3.28 (s, 3H), 3.26- 3.19 (m, 1H). | 468.3 | R-isomer: 100% ee; [α] = +42.112°; S-isomer: 96% ee; [α] = −39.840°; |
| 236 | 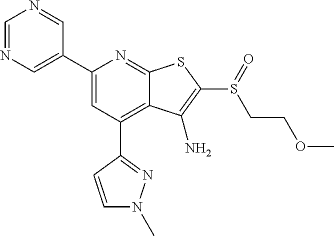 [SMILES: COCCS(=O)c1sc2nc(-c3cncnc3)cc(-c3ccn(C)n3)c2c1N] | DMSO-d6 9.60 (s, 2H), 9.31 (s, 1H), 8.29 (s, 1H), 8.03 (d, J = 2.0 Hz, 1H), 7.22 (d, J = 2.0 Hz, 1H), 7.05 (s, 2H), 4.02 (s, 3H), 3.78-3.71 (m, 1H), 3.66-3.59 (m, 1H), 3.43-3.36 (m, 1H), 3.29 (s, 3H), 3.28-3.20 (m, 1H). | 415.0 | |
| 237 | 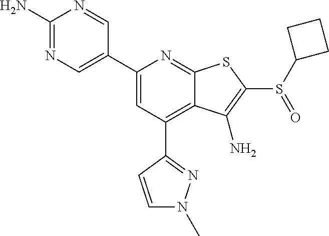 [SMILES: Cn1ccc(-c2cc(-c3cnc(N)nc3)nc3sc(S(=O)C4CCC4)c(N)c23)n1] | DMSO-d6 9.08 (s, 2H), 7.99 (s, 2H), 7.14 (s, 3H), 6.92 (s, 2H), 4.00 (s, 3H), 3.93-3.82 (s, 1H), 2.69-2.58 (m, 1H), 2.25-2.12 (m, 3H), 2.09- 1.88 (m, 2H). | 426.1 | |
| 238 | 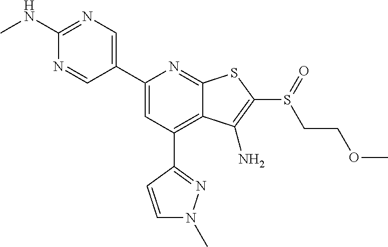 | DMSO-d6 9.12 (s, 2H), 8.03-7.99 (m, 2H), 7.66-7.60 (m, 1H), 7.15 (d, J = 2.4 Hz, 1H), 6.96 (s, 2H), 4.00 (s, 3H), 3.78-3.69 (m, 1H), 3.64- 3.57 (m, 1H), 3.36 (s, 1H), 3.29 (s, 3H), 3.25-3.17 (m, 1H), 2.90 (d, J = 4.8 Hz, 3H) | 444.1 | |
| 239 | 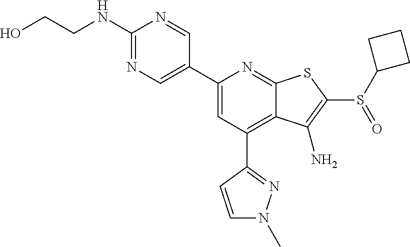 [SMILES: Cn1ccc(-c2cc(-c3cnc(NCCO)nc3)nc3sc(S(=O)C4CCC4)c(N)c23)n1] | DMSO-d6 9.10 (s, 2H), 8.01-7.97 (m, 2H), 7.60-7.55 (m, 1H), 7.13 (d, J = 2.4 Hz, 1H), 6.92 (s, 2H), 4.75-4.68 (m, 1H), 4.00 (s, 3H), 3.92-3.82 (m, 1H), 3.60-3.53 (m, 2H), 3.48-3.41 (m, 2H), 2.70- 2.60 (m, 1H), 2.23-2.13 (m, 3H), 2.06-1.88 (m, 2H). | 470.1 | |
| 240 | 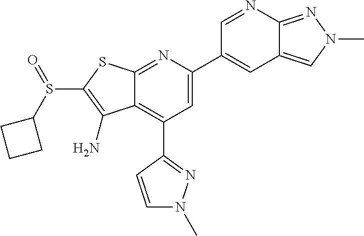 [SMILES: Cn1ccc(-c2cc(-c3cnc4nn(C)cc4c3)nc3sc(S(=O)C4CCC4)c(N)c23)n1] | DMSO-d6 9.44 (d, J = 2.0 Hz, 1H), 9.05 (d, J = 2.0 Hz, 1H), 8.57 (s, 1H), 8.19 (s, 1H), 8.01 (d, J = 2.4 Hz, 1H), 7.19 (d, J = 2.0 Hz, 1H), 6.95 (s, 2H), 4.25 (s, 3H), 4.01 (s, 3H), 3.93-3.84 (m, 1H), 2.69-2.60 (m, 1H), 2.25-2.13 (m, 3H), 2.08- 2.01 (m, 1H), 1.99-1.91 (m, 1H). | 464.2 | |
| 241 | 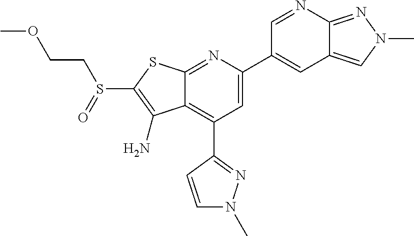 | DMSO-d6 9.45 (d, J = 2.0 Hz, 1H), 9.06 (d, J = 2.4 Hz, 1H), 8.58 (s, 1H), 8.21 (s, 1H), 8.02 (d, J = 2.0 Hz, 1H), 7.21 (d, J = 2.0 Hz, 1H), 6.99 (s, 2H), 4.25 (s, 3H), 4.01 (s, 3H), 3.73-3.72 (m, 1H), 3.63-3.61 (m, 1H), 3.38-3.37 (m, 1H), 3.36 (s, 3H), 3.29-3.23 (m, 1H). | 468.1 | R-isomer: 100% ee; [α] = +79.150. S-isomer: 100% ee; [α] = −82.080. |
| 242 | 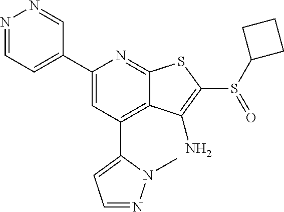 [SMILES: Cn1nccc1-c1cc(-c2ccnnc2)nc2sc(S(=O)C3CCC3)c(N)c12] | DMSO-d6 10.03 (m, 1H), 9.41 (m, 1H), 8.45 (m, 1H), 8.32 (s, 1H), 7.71 (d, J = 2.0 Hz, 1H), 6.67 (s, 1H), 5.07 (s, 2H), 3.94-3.86 (m, 1H), 3.73 (s, 3H), 2.68-2.66 (m, 1H), 2.24-2.16 (m, 3H), 2.15-1.93 (m, 2H). | 411.0 | |
| 243 | 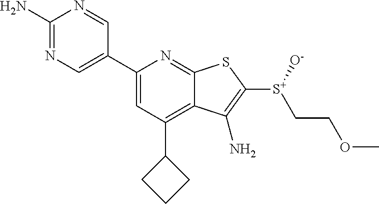 [SMILES: COCC[S@@+]([O-])c1sc2nc(-c3cnc(N)nc3)cc(C3CCC3)c2c1N] | 9.02 (s, 2H), 7.50 (s, 1H), 5.34 (s, 2H), 5.10 (s, 2H), 4.21-4.16 (m, 1H), 3.88- 3.86 (m, 1H), 3.70-3.69 (m, 1H), 3.67-3.61 (m, 1H), 3.41 (s, 3H), 3.28-3.22 (m, 1H), 2.52-2.42 (m, 4H), 2.41-2.39 (m, 1H), 2.16-2.00 (m, 1H). | 404.2 | |
| 244 | 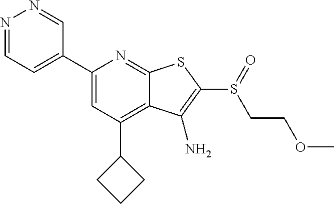 [SMILES: COCCS(=O)c1sc2nc(-c3ccnnc3)cc(C3CCC3)c2c1N] | 9.90 (dd, J1 = 2.4 Hz, J2 = 1.2 Hz, 1H), 9.36 (dd, J1 = 5.2 Hz, J2 = 1.2 Hz, 1H), 8.19 (dd, J1 = 5.6 Hz, J2 = 2.4 Hz, 1H), 7.78 (s, 1H), 5.18 (s, 2H), 4.31-4.22 (m, 1H), 3.95-3.88 (m, 1H), 3.76-3.69 (m, 1H), 3.68-3.60 (m, 1H), 3.44 (s, 3H), 3.35- 3.27 (m, 1H), 2.61-2.52 (m, 2H), 2.51-2.40 (m, 2H), 2.29-2.16 (m, 1H), 2.11- 2.00 (m, 1H). | 389.0 | |
| 245 | 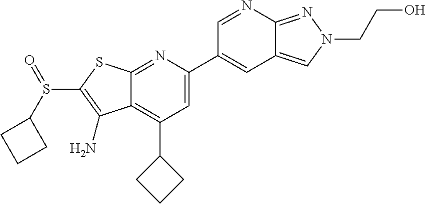 [SMILES: Nc1c(S(=O)C2CCC2)sc2nc(-c3cnc4nn(CCO)cc4c3)cc(C3CCC3)c12] | DMSO-d6 9.42 (d, J = 2.4 Hz, 1H), 9.03 (d, J = 2.4 Hz, 1H), 8.56 (s, 1H), 7.92 (s, 1H), 5.75 (s, 2H), 4.54 (t, J = 5.4 Hz, 2H), 4.47-4.39 (m, 1H), 3.93 (t, J = 5.4 Hz, 2H), 3.89-3.83 (m, 1H), 2.68-2.60 (m, 1H), 2.47-2.43 (m, 2H), 2.39-2.33 (m, 2H), 2.24-2.13 (m, 3H), 2.11-1.99 (m, 2H), 1.97-1.83 (m, 2H). | 468.1 | |
| 246 | 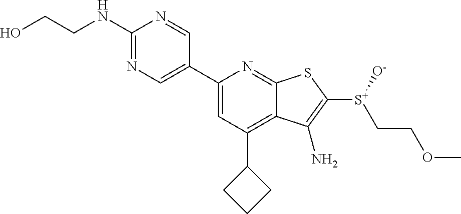 [SMILES: COCC[S@@+]([O-])c1sc2nc(-c3cnc(NCCO)nc3)cc(C3CCC3)c2c1N] | 8.97 (s, 2H), 7.44 (s, 1H), 5.98-5.91 (m, 1H), 5.10 (s, 2H), 4.24-4.13 (m, 1H), 3.95-3.86 (m, 3H), 3.82 (s, 1H), 3.73-3.66 (m, 3H), 3.65-3.58 (m, 1H), 3.42 (s, 3H), 3.31-3.23 (m, 1H), 2.56-2.46 (m, 2H), 2.45-2.30 (m, 2H), 2.24-2.11 (m, 1H), 2.05-1.95 (s, 1H) | 448.1 | R-isomer: 98.2% ee; [α] = +35.337; S-isomer: 94.8% ee; [α] = −36.966. |
| 247 | 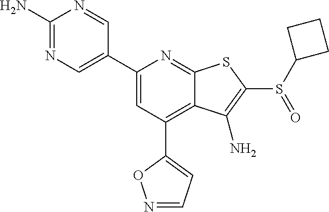 [SMILES: Nc1ncc(-c2cc(-c3ccno3)c3c(N)c(S(=O)C4CCC4)sc3n2)cn1] | 9.04 (s, 2H), 8.50 (d, J = 2.0 Hz, 1H), 7.68 (s, 1H), 6.76 (d, J = 1.6 Hz, 1H), 5.34 (s, 2H), 5.09 (s, 2H), 3.97 (t, J = 8.4 Hz, 1H), 2.86-2.79 (m, 1H), 2.43-2.32 (m, 2H), 2.32-2.20 (m, 1H), 2.14-2.03 (m, 2H). | 413.1 | |
| 248 | 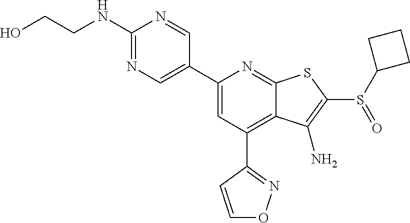 [SMILES: Nc1c(S(=O)C2CCC2)sc2nc(-c3cnc(NCCO)nc3)cc(-c3ccon3)c12] | DMSO-d6 9.29 (d, J = 1.6 Hz, 1H), 9.12 (s, 2H), 8.09 (s, 1H), 7.64 (t, J = 5.6 Hz, 1H), 7.39 (d, J = 2.0 Hz, 1H), 6.14 (s, 2H), 4.71 (t, J = 5.6 Hz, 1H), 3.90-3.86 (m, 1H), 3.58-3.53 (m, 2H), 3.46- 3.42 (m, 2H), 2.70-2.61 (m, 1H), 2.25-2.11 (m, 3H), 2.05-1.98 (m, 1H), 1.95-1.89 (m, 1H). | 457.2 | |
| 249 | 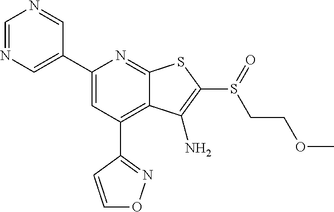 [SMILES: COCCS(=O)c1sc2nc(-c3cncnc3)cc(-c3ccon3)c2c1N] | DMSO-d6 9.62 (s, 2H), 9.34 (d, J = 1.6 Hz, 1H), 9.33 (s, 1H), 8.39 (s, 1H), 7.45 (d, J = 1.6 Hz, 1H), 6.26 (s, 2H), 3.80-3.73 (m, 1H), 3.69- 3.62 (m, 1H), 3.44-3.37 (m, 1H), 3.29 (s, 3H), 3.28- 3.23 (m, 1H). | 402.0 | |
| 250 | 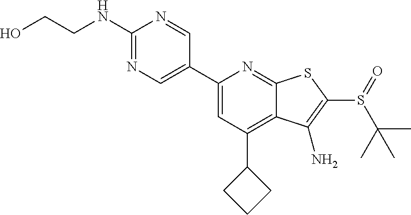 [SMILES: CC(C)(C)S(=O)c1sc2nc(-c3cnc(NCCO)nc3)cc(C3CCC3)c2c1N] | 8.92 (s, 2H), 7.38 (s, 1H), 6.16-6.13 (m, 1H), 5.25 (s, 2H), 4.22-4.14 (m, 1H), 3.98-3.88 (m, 2H), 3.76-3.64 (m, 2H), 2.54-2.37 (m, 3H), 2.34-2.25 (m, 1H), 2.20- 2.09 (m, 1H), 2.04-1.94 (m, 2H), 1.40 (s, 9H). | 446.1 | |
| 251 | 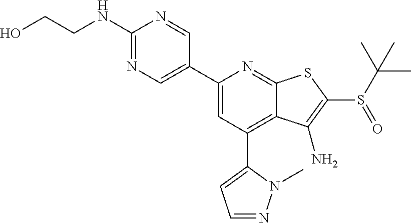 [SMILES: Cn1nccc1-c1cc(-c2cnc(NCCO)nc2)nc2sc(S(=O)C(C)(C)C)c(N)c12] | DMSO-d6 9.10 (s, 2H), 7.92 (s, 1H), 7.67 (d, J = 1.6 Hz, 1H), 7.66-7.61 (m, 1H), 6.63 (s, 1H), 4.89 (s, 2H), 4.73 (t, J = 5.6 Hz, 1H), 3.69 (s, 3H), 3.58-3.51 (m, 2H), 3.46-3.40 (m, 2H), 1.27 (s, 9H). | 472.1 | |
| 252 | 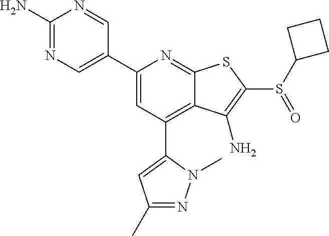 [SMILES: Cc1cc(-c2cc(-c3cnc(N)nc3)nc3sc(S(=O)C4CCC4)c(N)c23)n(C)n1] | 9.03 (s, 2H), 7.44 (s, 1H), 6.25 (d, J = 4.4 Hz, 1H), 5.31 (s, 2H), 4.64-4.55 (m, 2H), 3.96-3.92 (m, 1H), 3.66 (s, 3H), 2.89-2.75 (m, 1H), 2.42-2.31 (m, 5H), 2.30-2.20 (m, 1H), 2.15- 2.04 (m, 2H). | 440.1 | |
| 253 | 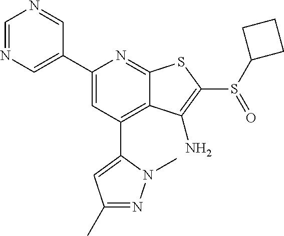 [SMILES: Cc1cc(-c2cc(-c3cncnc3)nc3sc(S(=O)C4CCC4)c(N)c23)n(C)n1] | 9.43 (s, 2H), 9.31 (s, 1H), 7.61 (s, 1H), 6.27 (s, 1H), 4.66 (d, J = 26.4 Hz, 2H), 4.03-3.88 (m, 1H), 3.68 (s, 3H), 2.90-2.78 (m, 1H), 2.48- 2.32 (m, 5H), 2.30-2.22 (m, 1H), 2.16-2.05 (m, 2H). | 425.2 | |
| 254 | 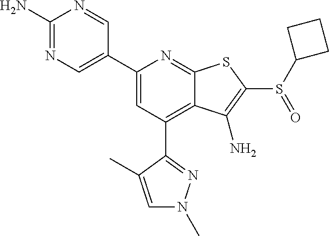 [SMILES: Cc1cn(C)nc1-c1cc(-c2cnc(N)nc2)nc2sc(S(=O)C3CCC3)c(N)c12] | DMSO-d6 9.03 (s, 2H), 7.82 (s, 1H), 7.77 (s, 1H), 7.14 (s, 2H), 6.00 (s, 2H), 3.93 (s, 3H), 3.87-3.81 (m, 1H), 2.66- 2.57 (m, 1H), 2.22-2.13 (m, 3H), 2.11 (s, 3H), 2.06- 1.97 (m, 1H), 1.97-1.88 (m, 1H) | 440.0 | |
| 255 | 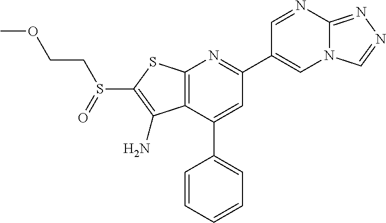 [SMILES: COCCS(=O)c1sc2nc(-c3cnc4nncn4c3)cc(-c3ccccc3)c2c1N] | DMSO-d6 10.21 (d, J = 2.8 Hz, 1H), 9.72 (d, J = 2.4 Hz, 1H), 8.77 (s, 1H), 8.17 (s, 1H), 7.63 (m, 5H), 5.02 (s, 2H), 3.78-3.71 (m, 1H), 3.68- 3.61 (m, 1H), 3.42-3.37 (m, H), 3.28 (s, 3H), 3.26-3.20 (m, 1H). | 451.1 | R-isomer: 100% ee; [α] = +40.213°; S-isomer: 100% ee; [α] = −75.514°; |
| 256 | 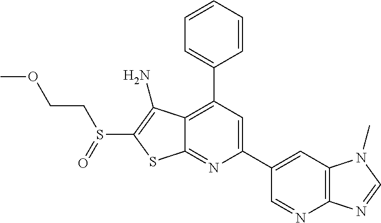 [SMILES: COCCS(=O)c1sc2nc(-c3cnc4ncn(C)c4c3)cc(-c3ccccc3)c2c1N] | 9.22 (d, J = 2.0 Hz, 1H), 8.62 (d, J = 2.4 Hz, 1H), 8.16 (s, 1H), 7.70 (s, 1H), 7.59-7.54 (m, 5H), 4.61 (s, 2H), 3.99 (s, 3H), 3.3.90- 3.85 (m, 1H), 3.74-3.68 (m, 1H), 3.64-3.58 (m, 1H), 3.41 (s, 3H), 3.32-3.25 (m, 1H). | 464.1 | R-isomer: 100% ee; [α] = +28.687°; S-isomer: 98% ee; [α] = −31.782°; |
| 257 | 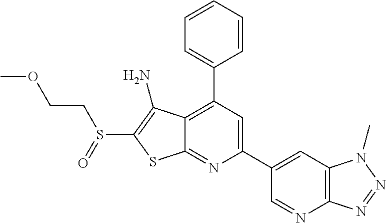 [SMILES: COCCS(=O)c1sc2nc(-c3cnc4nnn(C)c4c3)cc(-c3ccccc3)c2c1N] | DMSO-d6 9.58 (d, J = 2.0 Hz, 1H), 9.22 (d, J = 2.0 Hz, 1H), 8.13 (s, 1H), 7.64 (s, 5H), 5.01 (s, 2H), 4.42 (s, 3H), 3.78-3.72 (m, 1H), 3.67-3.62 (m, 1H), 3.41-3.31 (m, 1H), 3.29 (s, 3H), 3.27-3.20 (m, 1H). | 465.0 | |
| 258 | 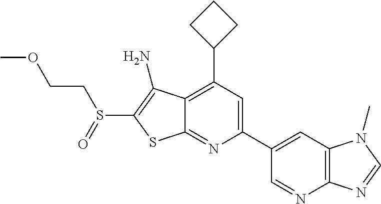 [SMILES: COCCS(=O)c1sc2nc(-c3cnc4ncn(C)c4c3)cc(C3CCC3)c2c1N] | 9.14 (d, J = 2.0 Hz, 1H), 8.34 (d, J = 2.0 Hz, 1H), 8.08 (s, 1H), 7.67 (s, 1H), 5.09 (s, 2H), 4.27-4.18 (m, 1H), 3.93 (s, 3H), 3.90-3.84 (m, 1H), 3.68-3.63 (m, 1H), 3.62-3.56 (m, 1H), 3.40 (s, 3H), 3.26-3.20 (m, 1H), 2.58-2.42 (m, 3H), 2.34- 2.27 (m, 1H), 2.21-2.12 (m, 1H), 2.03-1.99 (m, 1H). | 442.0 | R-isomer: 100% ee; [α] = +95.240°; S-isomer: 98% ee; [α] = −91.873°; |
| 259 | 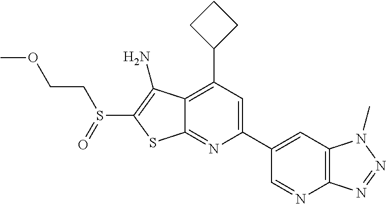 [SMILES: COCCS(=O)c1sc2nc(-c3cnc4nnn(C)c4c3)cc(C3CCC3)c2c1N] | DMSO-d6 9.54 (d, J = 2.0 Hz, 1H), 9.14 (d, J = 2.0 Hz, 1H), 8.06 (s, 1H), 5.80 (s, 2H), 4.52-4.44 (m, 4H), 3.78- 3.72 (m, 1H), 3.65-3.60 (m, 1H), 3.42-3.36 (m, 1H), 3.29 (s, 3H), 3.26-3.19 (m, 1H), 2.47-2.35 (m, 4H), 2.15-2.04 (m, 1H), 1.91- 1.83 (m, 1H). | 443.0 | |
| 260 | 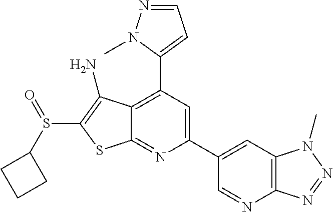 [SMILES: Cn1nccc1-c1cc(-c2cnc3nnn(C)c3c2)nc2sc(S(=O)C3CCC3)c(N)c12] | DMSO-d6 9.57 (d, J = 2.0 Hz, 1H), 9.22 (d, J = 2.4 Hz, 1H), 8.28 (s, 1H), 7.71 (d, J = 2.0 Hz, 1H), 6.68 (s, 1H), 5.05 (s, 2H), 4.42 (s, 3H), 3.93-3.85 (m, 1H), 3.73 (s, 3H), 2.68-2.63 (m, 1H), 2.26-2.12 (m, 3H), 2.08-1.99 (m, 1H), 1.96-1.88 (m, 1H). | 465.2 | |
| 261 | 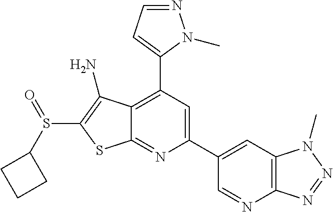 [SMILES: Cn1nccc1-c1cc(-c2cnc3nnn(C)c3c2)nc2sc(S(=O)C3CCC3)c(N)c12] | 9.21 (d, J = 2.0 Hz, 1H), 8.64 (d, J = 2.0 Hz, 1H), 8.19 (s, 1H), 7.75 (s, 1H), 7.69 (d, J = 2.0 Hz, 1H), 6.52 (m, 1H), 4.60-4.49 (m, 2H), 4.00 (s, 3H), 3.98- 3.92 (m, 1H), 3.78 (s, 3H), 2.89-2.77 (m, 1H), 2.47- 2.32 (m, 2H), 2.31-2.21 (m, 1H), 2.16-2.04 (m, 2H). | 464.1 | R-isomer: 100% ee; [α] = +77.141°; S-isomer: 97% ee; [α] = −59.110° |
| 262 | 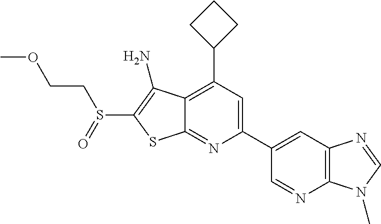 [SMILES: COCCS(=O)c1sc2nc(-c3cnc4c(c3)ncn4C)cc(C3CCC3)c2c1N] | 9.15 (d, J = 2.0 Hz, 1H), 8.67 (d, J = 2.0 Hz, 1H), 8.09 (s, 1H), 7.66 (s, 1H), 5.11 (s, 2H), 4.23-4.14 (m, 1H), 3.96 (s, 3H), 3.90- 3.84 (m, 1H), 3.70-3.65 (m, 1H), 3.63-3.57 (m, 1H), 3.40 (s, 3H), 3.30-3.24 (m, 1H), 2.55-2.48 (m, 2H), 2.42-2.35 (m, 1H), 2.22-2.10 (m, 2H), 2.05-1.96 (m, 1H) | 442.0 | R-isomer: 100% ee; [α] = +106.108°; S-isomer: 99% ee; [α] = −96.478°; |
| 263 | 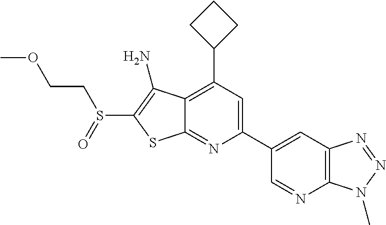 [SMILES: COCCS(=O)c1sc2nc(-c3cnc4c(c3)nnn4C)cc(C3CCC3)c2c1N] | DMSO-d6 9.54 (d, J = 6.0 Hz, 1H), 9.30 (d, J = 2.0 Hz, 1H), 8.01 (s, 1H), 5.76 (s, 2H), 4.47-4.38 (m, 1H), 4.35 (s, 3H), 3.78-3.73 (m, 1H), 3.64-3.59 (m, 1H), 3.42-3.36 (m, 1H), 3.29 (s, 3H), 3.25- 3.19 (m, 1H), 2.50- 2.36 (m, 4H), 2.13-2.02 (m, 1H), 1.90-1.82 (m, 1H). | 443.2 | |
| 264 | 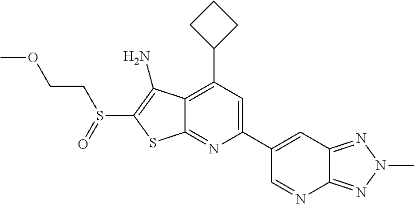 | DMSO-d6 9.60 (d, J = 2.0 Hz, 1H), 9.21 (d, J = 2.4 Hz, 1H), 8.04 (s, 1H), 5.77 (s, 2H), 4.60 (s, 3H), 4.50-4.41 (m, 1H), 3.78-.372 (m, 1H), 3.64-3.59 (m, 1H), 3.42-3.36 (m, 1H), 3.29 (s, 3H), 3.25- 3.19 (m, 1H), 2.46-2.35 (m, 4H), 2.14-2.02 (m, 1H), 1.90-1.82 (m, 1H). | 443.2 | |
| 265 | 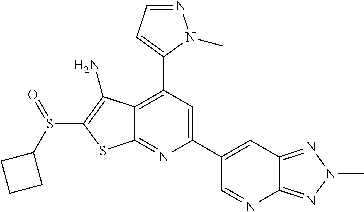 [SMILES: Cn1nc2cc(-c3cc(-c4ccnn4C)c4c(N)c(S(=O)C5CCC5)sc4n3)cnc2n1] | DMSO-d6 9.64 (d, J = 2.0 Hz, 1H), 9.21 (d, J = 2.4 Hz, 1H), 8.26 (s, 1H), 7.70 (d, J = 1.6 Hz, 1H), 6.66 (s, 1H), 5.03 (s, 2H), 4.59 (s, 3H), 3.93-3.85 (m, 1H), 3.74 (s, 3H), 2.74-2.61 (m, 1H), 2.26-2.13 (m, 3H), 2.06-1.99 (m, 1H), 1.97-1.87 (m, 1H). | 465.2 | |
| 266 | 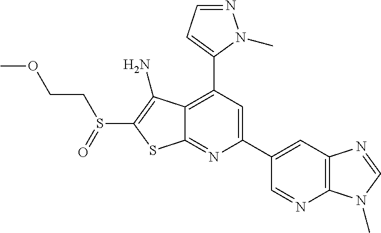 [SMILES: COCCS(=O)c1sc2nc(-c3cnc4c(c3)ncn4C)cc(-c3ccnn3C)c2c1N] | DMSO-d6 9.28 (d, J = 2.0 Hz, 1H), 8.89 (d, J = 2.0 Hz, 1H), 8.53 (s, 1H), 8.19 (s, 1H), 7.70 (d, J = 1.6 Hz, 1H), 6.67 (s, 1H), 5.05 (s, 2H), 3.91 (s, 3H), 3.79-3.71 (m, 4H), 3.67-3.60 (m, 1H), 3.38 (s, 1H), 3.29 (s, 3H), 3.26-3.19 (m, 1H). | 468.0 | |
| 267 | 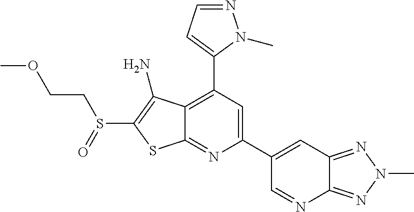 | DMSO-d6 9.65 (d, J = 2.0 Hz, 1H), 9.23 (d, J = 2.4 Hz, 1H), 8.28 (s, 1H), 7.71 (d, J = 2.0 Hz, 1H), 6.67 (s, 1H), 5.07 (s, 2H), 4.59 (s, 3H), 3.78-3.72 (m, 4H), 3.67- 3.62 (m, 1H), 3.42-3.38 (m, 1H), 3.28 (s, 3H), 3.26- 3.20 (m, 1H). | 469.2 | R-isomer: 98% ee; [α] = +106.322°; S-isomer: 95% ee; [α] = −98.728°; |
| 268 | 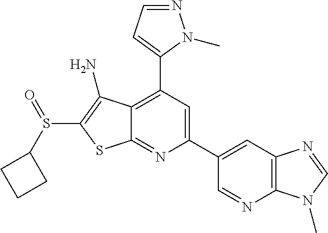 [SMILES: Cn1nccc1-c1cc(-c2cnc3c(c2)ncn3C)nc2sc(S(=O)C3CCC3)c(N)c12] | 9.19 (d, J = 2.0 Hz, 1H), 8.75 (d, J = 1.6 Hz, 1H), 8.13 (s, 1H), 7.69 (d, J = 1.6 Hz, 1H), 7.67 (s, 1H), 6.52 (m, 1H), 4.61-4.51 (m, 2H), 4.00 (s, 3H), 3.97- 3.93 (m, 1H), 3.79 (s, 3H), 2.89-2.79 (m, 1H), 2.47- 2.33 (m, 2H), 2.31-2.22 (m, 1H), 2.15-2.04 (m, 2H). | 464.0 | |
| 269 | 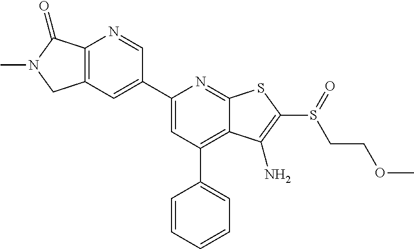 | 9.38 (s, 1H), 8.68 (d, J = 1.6 Hz, 1H), 7.70 (s, 1H), 7.61-7.56 (m, 3H), 7.53 (m, 2H), 4.63 (s, 2H), 4.50 (s, 2H), 3.87 (m, 1H), 3.74- 3.68 (m, 1H), 3.61 (m, 1H), 3.40 (s, 3H), 3.32 (s, 3H), 3.31-3.25 (m, 1H). | 479.2 | |
| 270 | 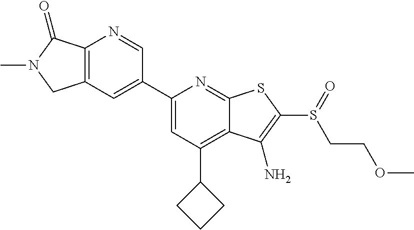 | 9.36 (d, J = 2.0 Hz, 1H), 8.55 (d, J = 1.6 Hz, 1H), 7.72 (s, 1H), 5.14 (s, 2H), 4.48 (s, 2H), 4.25 (m, 1H), 3.89 (m, 1H), 3.73-3.66 (m, 1H), 3.66-3.59 (m, 1H), 3.42 (s, 3H), 3.33 (s, 3H), 3.28 (m, 1H), 2.59-2.47 (m, 3H), 2.46-2.36 (m, 1H), 2.26-2.15 (m, 1H), 2.09-1.98 (m, 1H) | 457.2 | |
| 271 | 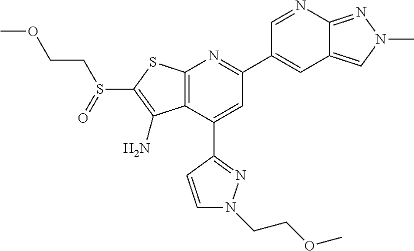 | 9.40 (d, J = 2.4 Hz, 1H), 8.84 (d, J = 2.4 Hz, 1H), 8.04 (s, 1H), 7.89 (s, 1H), 7.67 (d, J = 2.4 Hz, 1H), 6.76 (d, J = 2.4 Hz, 1H), 6.46 (s, 2H), 4.41 (t, J = 4.8 Hz, 2H), 4.31 (s, 3H), 3.90- 3.85 (m, 1H), 3.82-3.80 (m, 2H), 3.72-3.67 (m, 1H), 3.65-3.59 (m, 1H), 3.41 (s, 3H), 3.39 (s, 3H), 3.34- 3.27 (m, 1H). | 512.2 | R-isomer: 99% ee; [α] = +61.878°; S-isomer: 99% ee; [α] = −59.924°; |
| 272 | 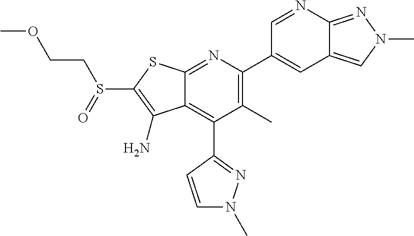 | 8.91 (d, J = 2.0 Hz, 1H), 8.29 (d, J = 2.0 Hz, 1H), 8.02 (s, 1H), 7.64 (d, J = 2.0 Hz, 1H), 6.48 (d, J = 2.0 Hz, 1H), 4.82 (s, 2H), 4.33 (s, 3H), 4.07 (s, 3H), 3.90- 3.82 (m, 1H), 3.71-3.64 (m, 1H), 3.62-3.55 (m, 1H), 3.40 (s, 3H), 3.31-3.23 (m, 1H), 2.25 (s, 3H). | 482.2 | |
| 273 | 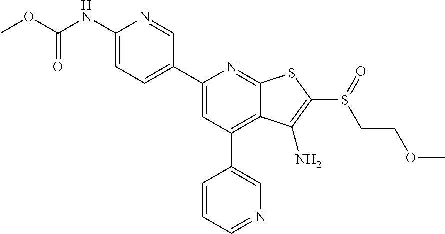 | DMSO-d6 10.51 (s, 1H), 9.13 (d, J = 1.6 Hz, 1H), 8.81 (d, J = 1.6 Hz, 1H), 8.77-8.76 (m, 1H), 8.62-8.59 (m, 1H), 8.05-8.03 (m, 1H), 8.00- 7.97 (m, 2H), 7.62-7.59 (m, 1H), 4.94 (s, 2H), 3.77- 3.72 (m, 1H), 3.70 (s, 3H), 3.66-3.61 (m, 1H), 3.41- 3.37 (m, 1H), 3.28 (s, 3H), 3.25-3.19 (m, 1H). | 484.1 | |
| 274 | 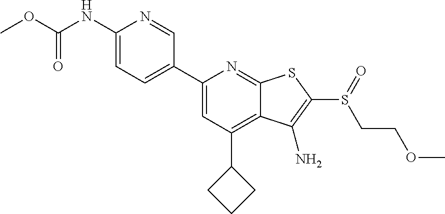 | 9.00 (d, J = 2.0 Hz, 1H), 8.50- 8.32 (m, 2H), 8.14 (d, J = 8.8 Hz, 1H), 7.61 (s, 1H), 5.12 (s, 2H), 4.26-4.18 (m, 1H), 3.93-3.91 (m, 1H), 3.89 (s, 3H), 3.76-3.68 (m, 1H), 3.66-3.60 (m, 1H), 3.43 (s, 3H), 3.33-3.25 (m, 1H), 2.57-2.35 (m, 4H), 2.25-2.16 (m, 1H), 2.06- 1.98 (m, 1H). | 461.1 | |
| 275 | 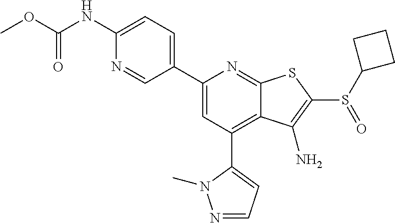 [SMILES: COC(=O)Nc1ccc(-c2cc(-c3ccnn3C)c3c(N)c(S(=O)C4CCC4)sc3n2)cn1] | 9.02 (s, 1H), 8.45-8.42 (m, 1H), 8.25 (m, 1H), 8.15- 8.13 (m, 1H), 7.67 (s, 1H), 7.57 (s, 1H), 6.51-6.48 (m, 1H), 4.57-4.47 (m, 2H), 3.97-3.91 (m, 1H), 3.86 (s, 3H), 3.75 (s, 3H), 2.85- 2.80 (m, 1H), 2.40-2.35 (m, 2H), 2H), 2.29-2.26 (m, 1H), 2.15-2.04 (m, 2H). | 483.1 | |
| 276 | 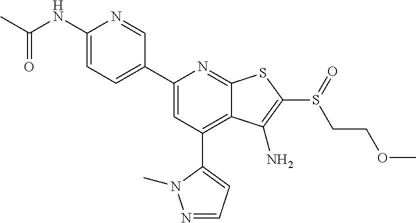 [SMILES: COCCS(=O)c1sc2nc(-c3ccc(NC(C)=O)nc3)cc(-c3ccnn3C)c2c1N] | 9.05-9.01 (m, 1H), 8.48- 8.42 (m, 1H), 8.39-8.34 (m, 1H), 8.10 (s, 1H), 7.70 (d, J = 2.0 Hz, 1H), 7.61 (s, 1H), 6.54-6.47 (m, 1H), 4.60-4.47 (m, 2H), 3.94-3.86 (m, 1H), 3.78 (s, 3H), 3.76- 3.68 (m, 1H), 3.66-3.58 (m, 1H), 3.42 (s, 3H), 3.33- 3.26 (m, 1H), 2.28 (s, 3H) | 471.1 | |
| 277 | 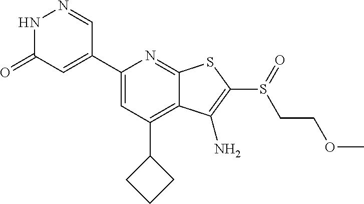 [SMILES: COCCS(=O)c1sc2nc(-c3cn[nH]c(=O)c3)cc(C3CCC3)c2c1N] | 11.05-10.79 (m, 1H), 8.69 (d, J = 2.0 Hz, 1H), 7.66 (s, 1H), 7.49 (d, J = 2.0 Hz, 1H), 5.16 (s, 2H), 4.27- 4.18 (m, 1H), 3.92-3.87 (m, 1H), 3.73-3.68 (m, 1H), 3.66-3.60 (m, 1H), 3.42 (s, 3H), 3.33-3.27 (m, 1H), 2.57-2.50 (m, 2H), 2.45- 2.34 (m, 2H), 2.25-2.16 (m, 1H), 2.08-1.99 (m, 1H). | 405.0 | |
| 278 | 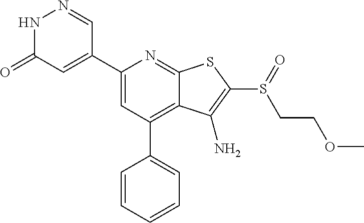 [SMILES: COCCS(=O)c1sc2nc(-c3cn[nH]c(=O)c3)cc(-c3ccccc3)c2c1N] | 10.50-10.46 (m, 1H), 8.69 (d, J = 2.0 Hz, 1H), 7.59- 7.57 (m, 4H), 7.49-7.47 (m, 3H), 4.65 (s, 2H), 3.90- 3.85 (m, 1H), 3.73-3.68 (m, 1H), 3.64-3.58 (m, 1H), 3.40 (s, 3H), 3.33-3.26 (m, 1H) | 427.0 | |
| 279 | 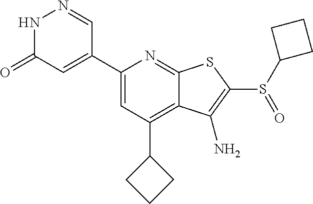 [SMILES: Nc1c(S(=O)C2CCC2)sc2nc(-c3cn[nH]c(=O)c3)cc(C3CCC3)c12] | 10.73-10.69 (m, 1H), 8.69 (d, J = 2.4 Hz, 1H), 7.66 (s, 1H), 7.49 (d, J = 2.0 Hz, 1H), 5.20 (s, 2H), 4.29- 4.20 (m, 1H), 4.02-3.94 (m, 1H), 2.89-2.79 (m, 1H), 2.57-2.48 (m, 2H), 2.46- 2.33 (m, 4H), 2.31-2.23 (m, 1H), 2.21-2.15 (m, 1H), 2.13-1.99 (m, 3H). | 401.0 | |
| 280 | 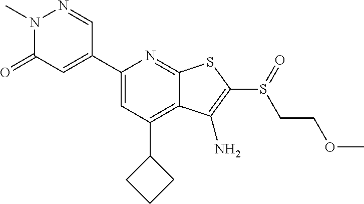 [SMILES: COCCS(=O)c1sc2nc(-c3cnn(C)c(=O)c3)cc(C3CCC3)c2c1N] | DMSO-d6 8.67 (d, J = 2.0 Hz, 1H), 7.97 (s, 1H), 7.77 (d, J = 2.4 Hz, 1H), 5.79 (s, 2H), 4.48- 4.39 (m, 1H), 3.77-3.72 (m, 4H), 3.63-3.58 (m, 1H), 3.41-3.36 (m, 1H), 3.28 (s, 3H), 3.25-3.18 (m, 1H), 2.44-2.32 (m, 4H), 2.09- 2.17 (m, 1H), 1.87-1.79 (m, 1H). | 419.0 | |
| 281 | 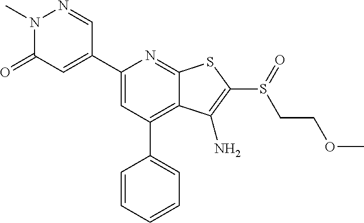 [SMILES: COCCS(=O)c1sc2nc(-c3cnn(C)c(=O)c3)cc(-c3ccccc3)c2c1N] | 8.67-8.65 (m, 1H), 7.62- 7.58 (m, 3H), 7.57 (s, 1H), 7.52-7.46 (m, 3H), 4.66 (s, 2H), 3.92-3.85 (m, 4H), 3.75-3.68 (m, 1H), 3.66- 3.59 (m, 1H), 3.42 (s, 3H), 3.34-3.26 (m, 1H). | 441.1 | |
| 282 | 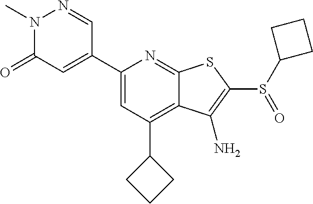 [SMILES: Cn1ncc(-c2cc(C3CCC3)c3c(N)c(S(=O)C4CCC4)sc3n2)cc1=O] | 8.64 (d, J = 2.0 Hz, 1H), 7.65 (s, 1H), 7.48 (d, J = 2.4 Hz, 1H), 5.19 (s, 2H), 4.28- 4.19 (m, 1H), 4.02-3.94 (m, 1H), 3.88 (s, 3H), 2.88- 2.79 (m, 1H), 2.56-2.47 (m, 2H), 2.46-2.35 (m, 4H), 2.31-2.25 (m, 1H), 2.23- 2.15 (m, 1H), 2.11-2.00 (m, 3H). | 403.1 | |
| 283 | 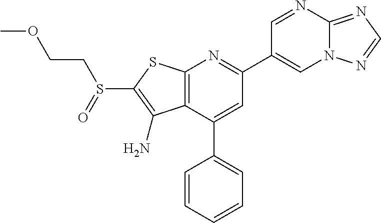 [SMILES: COCCS(=O)c1sc2nc(-c3cnc4ncnn4c3)cc(-c3ccccc3)c2c1N] | 9.69-9.63 (m, 1H), 9.58- 9.52 (m, 1H), 8.59 (s, 1H), 7.63 (s, 1H), 7.63-7.59 (m, 3H), 7.52 (m, 2H), 4.64 (s, 2H), 3.92-3.84 (m, 1H), 3.74-3.69 (m, 1H), 3.65-3.59 (m, 1H), 3.41 (s, 3H), 3.30 (m, 1H). | 451.1 | |
| 284 | 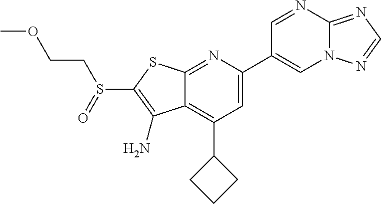 [SMILES: COCCS(=O)c1sc2nc(-c3cnc4ncnn4c3)cc(C3CCC3)c2c1N] | 9.62 (d, J = 2.4 Hz, 1H), 9.57 (d, J = 2.4 Hz, 1H), 8.59 (s, 1H), 7.70 (s, 1H), 5.15 (s, 2H), 4.29-4.23 (m, 1H), 3.93-3.87 (m, 1H), 3.74-3.69 (m, 1H), 3.66-3.60 (m, 1H), 3.43 (s, 3H), 3.33- 3.27 (m, 1H), 2.60-2.51 (m, 2H), 2.49-2.39 (m, 2H), 2.27-2.16 (m, 1H), 2.10- 2.01 (m, 1H). | 429.2 | |
| 285 | 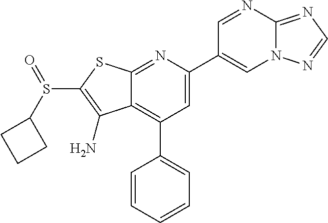 [SMILES: Nc1c(S(=O)C2CCC2)sc2nc(-c3cnc4ncnn4c3)cc(-c3ccccc3)c12] | DMSO-d6 10.19 (d, J = 2.4 Hz, 1H), 9.71 (d, J = 2.4 Hz, 1H), 8.76 (s, 1H), 8.14 (s, 1H), 7.62 (brs, 5H), 4.99 (s, 2H), 3.90-3.83 (m, 1H), 2.65- 2.60 (m, 1H), 2.25-2.11 (m, 3H), 2.07-1.98 (m, 1H), 1.92 (m, 1H). | 447.1 | R-isomer: 99% ee; [α] = +114.673°; S-isomer: 98% ee; [α] = −131.825° |
| 286 | 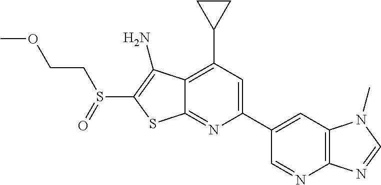 [SMILES: COCCS(=O)c1sc2nc(-c3cnc4ncn(C)c4c3)cc(C3CC3)c2c1N] | DMSO-d6 9.25 (d, J = 2.0 Hz, 1H), 8.77 (d, J = 2.0 Hz, 1H), 8.52 (s, 1H), 7.74 (s, 1H), 6.03 (s, 2H), 3.98 (s, 3H), 3.79-3.73 (m, 1H), 3.67- 3.62 (m, 1H), 3.42-3.36 (m, 1H), 3.31 (s, 3H), 3.26-3.20 (m, 1H), 2.73-2.66 (m, 1H), 1.20-1.12 (m, 4H). | 428.2 | R-isomer: 100% ee; [α] = +37.928°; S-isomer: 98% ee; [α] = −47.481°; |
| 287 | 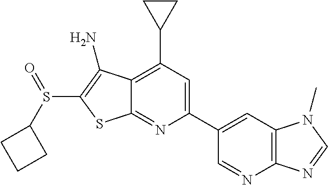 [SMILES: Cn1cnc2ncc(-c3cc(C4CC4)c4c(N)c(S(=O)C5CCC5)sc4n3)cc21] | 9.14 (d, J = 2.4 Hz, 1H), 8.51 (d, J = 2.0 Hz, 1H), 8.14 (s, 1H), 7.55 (s, 1H), 5.54 (s, 2H), 4.02-3.94 (m, 4H), 2.89-2.79 (m, 1H), 2.60-2.54 (m, 1H), 2.46-2.35 (m, 2H), 2.31-2.21 (m, 1H), 2.15-2.06 (m, 2H), 1.25- 1.20 (m, 2H), 1.16-1.11 (m, 1H), 1.07-1.01 (m, 1H). | 424.0 | R-isomer: 100% ee; [α] = +84.195°; S-isomer: 97% ee; [α] = −78.173°; |
| 288 | 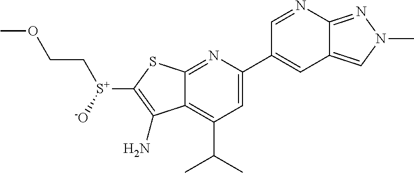 | 9.37 (d, J = 2.0 Hz, 1H), 8.74 (d, J = 2.0 Hz, 1H), 8.03 (s, 1H), 7.69 (s, 1H), 5.09 (s, 2H), 4.31 (s, 3H), 3.94-3.87 (m, 1H), 3.84- 3.75 (m, 1H), 3.74-3.60 (m, 2H), 3.43 (s, 3H), 3.34- 3.26 (m, 1H), 1.50 (m, 6H). | 430.2 | R-isomer: 100% ee; [α] = +30.365°; S-isomer: 98.9% ee; [α] = −45.960° |
| 289 | 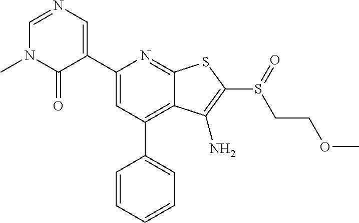 | 9.08 (s, 1H), 8.42 (s, 1H), 8.27 (s, 1H), 7.53 (s, 5H), 4.63 (s, 2H), 3.90-3.85 (m, 1H), 3.73-3.68 (m, 1H), 3.65 (s, 3H), 3.62-3.59 (m, 1H), 3.41 (s, 3H), 3.32- 3.26 (m, 1H). | 441.0 | |
| 290 | 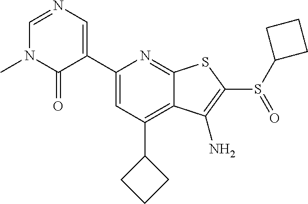 [SMILES: Cn1cncc(-c2cc(C3CCC3)c3c(N)c(S(=O)C4CCC4)sc3n2)c1=O] | 9.00 (s, 1H), 8.53 (s, 1H), 8.25 (s, 1H), 5.13 (s, 2H), 4.23-4.15 (m, 1H), 4.00- 3.92 (m, 1H), 3.66 (s, 3H), 2.86-2.79 (m, 1H), 2.50- 2.42 (m, 4H), 2.40-2.32 (m, 2H), 2.29-2.20 (m, 1H), 2.16-2.05 (m, 3H), 2.02-1.95 (m, 1H). | 415.0 | |
| 291 | 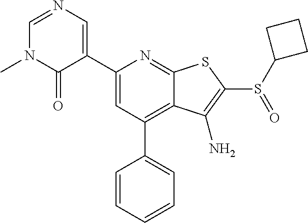 [SMILES: Cn1cncc(-c2cc(-c3ccccc3)c3c(N)c(S(=O)C4CCC4)sc3n2)c1=O] | 9.06 (s, 1H), 8.39 (s, 1H), 8.25 (s, 1H), 7.52-7.50 (m, 5H), 4.60 (s, 2H), 4.01- 3.93 (m, 1H), 3.63 (s, 3H), 2.84-2.77 (m, 1H), 2.43- 2.33 (m, 2H), 2.30-2.18 (m, 1H), 2.13-2.02 (m, 2H). | 437.2 | |
| 292 | 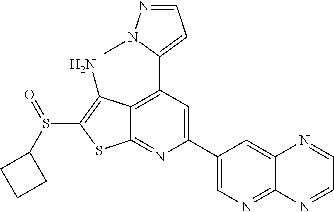 [SMILES: Cn1nccc1-c1cc(-c2cnc3nccnc3c2)nc2sc(S(=O)C3CCC3)c(N)c12] | 10.02 (d, J = 2.4 Hz, 1H), 9.13 (d, J = 2.0 Hz, 1H), 9.12 (d, J = 2.4 Hz, 1H), 9.04 (d, J = 1.6 Hz, 1H), 7.86 (s, 1H), 7.72 (d, J = 2.0 Hz, 1H), 6.55 (s, 1H), 4.69-4.53 (m, 2H), 4.04- 3.94 (m, 1H), 3.81 (s, 3H), 2.93-2.79 (m, 1H), 2.49- 2.25 (m, 3H), 2.21-2.03 (m, 2H). | 462.2 | |
| 293 | 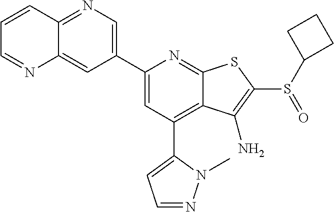 [SMILES: Cn1nccc1-c1cc(-c2cnc3cccnc3c2)nc2sc(S(=O)C3CCC3)c(N)c12] | 9.81 (d, J = 2.4 Hz, 1H), 9.07 (dd, J1 = 4.4 Hz, J2 = 1.6 Hz, 1H), 9.02 (d, J = 2.4 Hz, 1H), 8.50 (d, J = 8.4 Hz, 1H), 7.83 (s, 1H), 7.76- 7.69 (m, 2H), 6.55 (s, 1H), 4.71-4.51 (m, 2H), 4.04-3.93 (m, 1H), 3.81 (s, 3H), 2.92-2.80 (m, 1H), 2.50-2.23 (m, 3H), 2.20-2.03 (m, 2H). | 461.2 | |
| 294 | 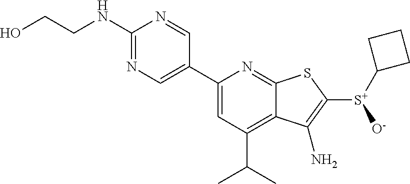 [SMILES: CC(C)c1cc(-c2cnc(NCCO)nc2)nc2sc([S@+]([O-])C3CCC3)c(N)c12] | | 432.1 | R-isomer: 100% ee; [α] = +177.876° |

The following compounds were prepared from known starting materials in a manner similar to that used for EXAMPLE 25. For select compounds, the enantiomers were resolved from the racemate using chiral supercritical fluid chromatography (SFC) and are reported with enantiomeric excess (ee) and/or optical rotation values. In some cases, only the more active R-(+) isomer was prepared/isolated.

| | | | LCMS | |
|---|---|---|---|---|
| Ex. | | | (ES⁺; | |
| No. | Structure | 400 MHz NMR (δ; CDCl₃ unless otherwise noted) | MH⁺) | Chiral info |
| 295 | 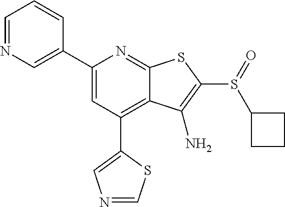 [SMILES: Nc1c(S(=O)C2CCC2)sc2nc(-c3cccnc3)cc(-c3cncs3)c12] | DMSO-d6 9.36-9.38 (m, 2 H), 8.67 (d, J = 6.4 Hz, 1H), 8.56 (dd, J1 = 2.4 Hz, J2 = 8.0 Hz, 1H), 8.20 (s, 1H), 8.05 (s, 1H), 7.53-7.56 (m, 1H), 5.19 (s, 2H), 3.85-3.93 (m, 1H), 2.63-2.70 (m, 1H), 2.14-2.23 (m, 3H), 1.90-2.03 (m, 2H). | 413.0 | |
| 296 | 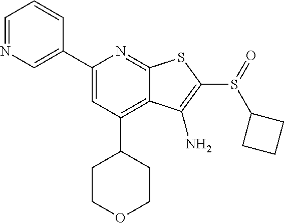 [SMILES: Nc1c(S(=O)C2CCC2)sc2nc(-c3cccnc3)cc(C3CCOCC3)c12] | 9.14 (d, J = 1.6 Hz, 1H), 8.58 (d, J = 6.4 Hz, 1H), 8.26- 8.29 (m, 1H), 7.51 (s, 1H), 7.31-7.34 (m, 1H), 4.96 (s, 2H), 4.06-4.11 (m, 2H), 3.82-3.90 (m, 1H), 3.51-3.59 (m, 3H), 2.68-2.78 (m, 1H), 2.12-2.37 (m, 3H), 1.95-2.04 (m, 2H), 1.86-1.93 (m, 4H). | 414.2 | |
| 297 | 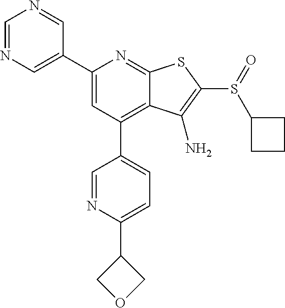 [SMILES: Nc1c(S(=O)C2CCC2)sc2nc(-c3cncnc3)cc(-c3ccc(C4COC4)nc3)c12] | 9.44 (s, 2H), 9.31 (s, 1H), 8.83 (d, J = 2.0 Hz, 1H), 7.85 (dd, J₁ = 2.4 Hz, J₂ = 8.0 Hz, 1H), 7.57 (s, 1H), 7.48 (d, J = 8.0 Hz, 1H), 5.18-5.14 (m, 2H), 5.03 (t, J = 6.0 Hz, 2H), 4.58 (s, 2H), 4.57-4.42 (m, 1H), 4.02-3.94 (m, 1H), 2.87-2.81 (m, 1H), 2.43-2.38 (m, 3H), 2.13-2.08 (m, 1H). | 464.1 | |
| 298 | 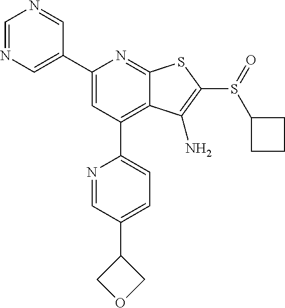 [SMILES: Nc1c(S(=O)C2CCC2)sc2nc(-c3cncnc3)cc(-c3ccc(C4COC4)cn3)c12] | 9.46 (s, 2H), 9.32 (s, 1H), 8.76 (d, J = 2.4 Hz, 1H), 8.18 (dd, J₁ = 2.4 Hz, J₂ = 8.4 Hz 1H), 7.83 (d, J = 8.0 Hz, 1H), 7.74 (s, 1H), 5.69 (m, 2H), 5.26-5.22 (m, 2H), 4.83 (t, J = 8.0 Hz, 1H), 4.42-4.37 (m, 1H), 3.99-3.88 (m, 1H), 2.45-2.36 (m, 2H), 2.35-2.21 (m, 1H), 2.14-2.07 (m, 2H). | 464.1 | |
| 299 | 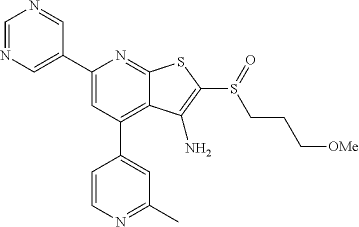 [SMILES: COCCCS(=O)c1sc2nc(-c3cncnc3)cc(-c3ccnc(C)c3)c2c1N] | DMSO-d6 9.58 (s, 2H), 9.30 (s, 1H), 8.65 (d, J = 4.8 Hz, 1H), 8.09 (s, 1H), 7.53 (s, 1H), 7.44 (d, J = 5.2 Hz, 1H), 5.02 (s, 2H), 3.43 (t, J = 6.4 Hz, 2H), 3.23 (s, 3H), 3.21-314 (m, 1H), 3.11-3.05 (m, 1H), 2.59 (s, 3H), 1.89-1.82 (m, 2H) | 440.0 | R-isomer: 100% ee; [α] = +65.7° S-isomer: 100% ee; [α] = −49.5° |
| 300 | 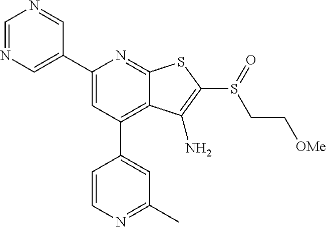 [SMILES: COCCS(=O)c1sc2nc(-c3cncnc3)cc(-c3ccnc(C)c3)c2c1N] | DMSO-d6 9.58 (s, 2H), 9.29 (s, 1H), 8.65 (d, J = 4.8 Hz, 1H), 8.09 (s, 1H), 7.52 (s, 1H), 7.44 (d, J = 5.2 Hz, 1H), 5.02 (s, 2H), 3.78-3.72 (m, 1H), 3.66-3.61 (m, 1H), 3.42-3.36 (m, 1H), 3.28 (s, 3H), 3.27-3.20 (m, 1H), 2.59 (s, 3H) | 426.0 | |
| 301 | 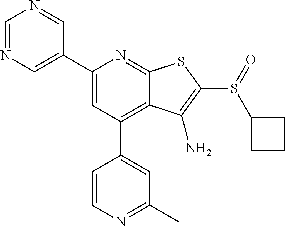 [SMILES: Cc1cc(-c2cc(-c3cncnc3)nc3sc(S(=O)C4CCC4)c(N)c23)ccn1] | 9.41 (s, 2H), 9.28 (s, 1H), 8.71-8.70 (m, 1H), 7.53 (s, 1H), 7.31-7.27 (m, 1H), 7.26 (s, 1H), 4.61 (s, 2H), 4.00- 3.92 (m, 1H), 2.84-2.77 (m, 1H), 2.69 (s, 3H), 2.27-2.25 (m, 2H), 2.11-2.09 (m, 1H), 2.08-2.07 (m, 2H). | 422.0 | |

The following compounds were prepared from known starting materials in a manner similar to that used for EXAMPLE 7. In some cases, only the more active R-(+) isomer was prepared/isolated.

| Ex. | | 400 MHz NMR (δ; CDCl₃ | LCMS | |
|---|---|---|---|---|
| No. | Structure | unless otherwise noted) | (ES⁺; MH⁺) | Chiral info |
| 302 | 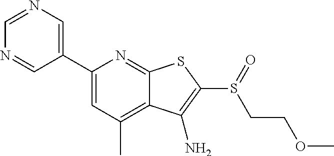 [SMILES: COCCS(=O)c1sc2nc(-c3cncnc3)cc(C)c2c1N] | 9.39 (s, 2H), 9.29 (s, 1H), 7.49 (s, 1H), 5.15 (s, 2H), 3.91 (m, 1H), 3.77-3.69 (m, 1H), 3.66-3.59 (m, 1H), 3.44 (s, 3H), 3.31 (m, 1H), 2.91 (s, 3H) | 349.1 | |
| 303 | 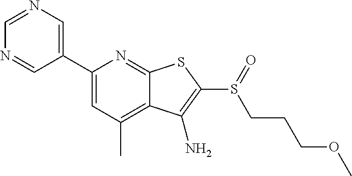 [SMILES: COCCCS(=O)c1sc2nc(-c3cncnc3)cc(C)c2c1N] | 9.41 (s, 2H), 9.30 (s, 1H), 7.50 (s, 1H), 5.20 (s, 2H), 3.59-3.53 (m, 2H), 3.43-3.35 (m, 4H), 3.32-3.24 (m, 1H), 2.91 (s, 3H), 2.14-2.04 (m, 2H). | 363.2 | |
| 304 | 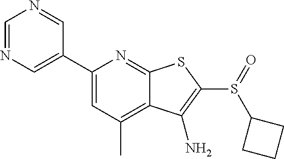 [SMILES: Cc1cc(-c2cncnc2)nc2sc(S(=O)C3CCC3)c(N)c12] | 9.39 (s, 2H), 9.28 (s, 1H), 7.48 (s, 1H), 5.17 (s, 2H), 3.98 (m, 1H), 2.89 (s, 3H), 2.87-2.77 (m, 1H), 2.47- 2.33 (m, 2H), 2.32-2.22 (m, 1H), 2.16-2.04 (m, 2H). | 345.1 | R-isomer: 100% ee; [α] = +175.431°; S-isomer: 100% ee; [α] = −195.207°; |
| 305 | 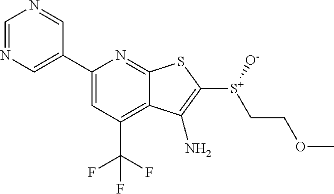 [SMILES: COCC[S@@+]([O-])c1sc2nc(-c3cncnc3)cc(C(F)(F)F)c2c1N] | 9.47 (s, 2H), 9.35 (s, 1H), 8.06 (s, 1H), 5.29 (s, 2H), 3.95-3.88 (m, 1H), 3.76- 3.65 (m, 2H), 3.43 (s, 3H), 3.39-3.31 (m, 1H). | 403.0 | R-isomer: 100% ee; [α] = +73.873°; |
| 306 | 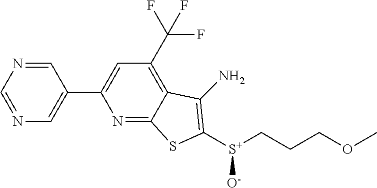 [SMILES: COCCC[S@@+]([O-])c1sc2nc(-c3cncnc3)cc(C(F)(F)F)c2c1N] | 9.47 (s, 2H), 9.35 (s, 1H), 8.06 (s, 1H), 5.34 (s, 2H), 3.57-3.55 (m, 2H), 3.48- 3.41 (m, 1H), 3.37 (s, 3H), 3.35-3.28 (m, 1H), 2.18- 2.01 (m, 2H). | 417.0 | R-isomer: 100% ee; [α] = +75.521° |
| 307 | 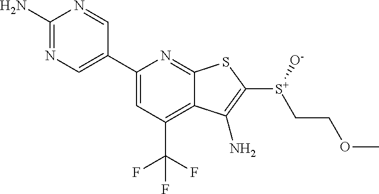 [SMILES: COCC[S@@+]([O-])c1sc2nc(-c3cnc(N)nc3)cc(C(F)(F)F)c2c1N] | 9.08 (s, 2H), 7.91 (s, 1H), 5.39 (s, 2H), 5.24 (s, 2H), 3.95-3.89 (m, 1H), 3.76- 3.65 (m, 2H), 3.44 (s, 3H), 3.38-3.30 (m, 1H) | 418.0 | R-isomer: 100% ee; [α] = +44.975°; S-isomer: 100% ee; [α] = −44.803° |
| 308 | 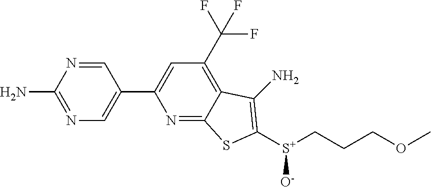 [SMILES: COCCC[S@@+]([O-])c1sc2nc(-c3cnc(N)nc3)cc(C(F)(F)F)c2c1N] | 9.08 (s, 2H), 7.90 (s, 1H), 5.37 (s, 2H), 5.29 (s, 2H), 3.58-3.54 (m, 2H), 3.48- 3.40 (m, 1H), 3.38 (s, 3H), 3.34-3.28 (m, 1H), 2.14- 2.05 (m, 2H). | 432.0 | R-isomer: 100% ee; [α] = +124.298°; S-isomer: 100% ee; [α] = −122.651° |
| 309 | 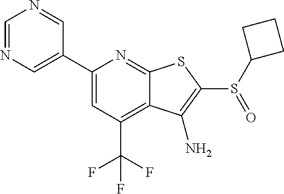 [SMILES: Nc1c(S(=O)C2CCC2)sc2nc(-c3cncnc3)cc(C(F)(F)F)c12] | 9.46 (s, 2H), 9.35 (s, 1H), 8.05 (s, 1H), 5.28 (s, 2H), 4.07-4.00 (m, 1H), 2.92- 2.80 (m, 1H), 2.52-2.24 (m, 3H), 2.07 (s, 2H) | 399.0 | |
| 310 | 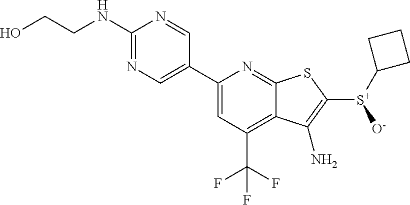 [SMILES: Nc1c([S@+]([O-])C2CCC2)sc2nc(-c3cnc(NCCO)nc3)cc(C(F)(F)F)c12] | 9.04 (s, 2H), 7.85 (s, 1H), 5.89-5.86 (m, 1H), 5.21 (s, 2H), 4.05-3.97 (m, 1H), 3.92-3.88 (m, 2H), 3.74- 3.70 (m, 2H), 3.16 (t, J = 5.6 Hz, 1H), 2.89-2.80 (m, 1H), 2.49-2.20 (m, 2H), 2.17-2.04 (m, 2H). | 458.0 | R-isomer: 100% ee; [α] = +80.486°; |
| 311 | 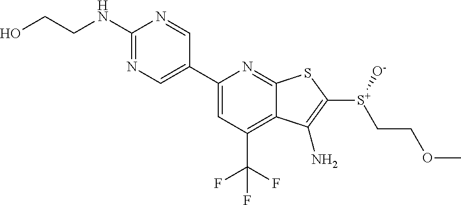 [SMILES: COCC[S@@+]([O-])c1sc2nc(-c3cnc(NCCO)nc3)cc(C(F)(F)F)c2c1N] | 9.07 (s, 2H), 7.88 (s, 1H), 5.88 (t, J = 5.6 Hz, 1H), 5.24 (s, 2H), 3.95-3.89 (m, 3H), 3.76-3.65 (m, 4H), 3.44 (s, 3H), 3.38-3.28 (m, 1H), 3.12 (t, J = 4.8 Hz, 1H) | 462.0 | R-isomer: 100% ee; [α] = +39.407°; S-isomer: 100% ee; [α] = −46.148°; |
| 312 | 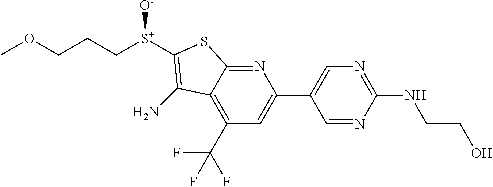 [SMILES: COCCC[S@@+]([O-])c1sc2nc(-c3cnc(NCCO)nc3)cc(C(F)(F)F)c2c1N] | 9.06 (s, 2H), 7.88 (s, 1H), 5.92-5.88 (m, 1H), 5.28 (s, 2H), 3.95-3.88 (m, 2H), 3.76-3.70 (m, 2H), 3.56 (t, J = 5.6 Hz, 2H), 3.48- 3.40 (m, 1H), 3.38 (s, 3H), 3.34-3.26 (m, 1H), 3.16- 3.07 (m, 1H), 2.09 (m, 2H). | 476.0 | R-isomer: 100% ee; [α] = +11.507°; S-isomer: 99% ee; [α] = −11.463°; |
| 313 | 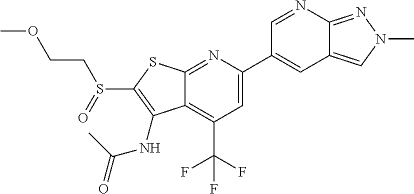 | 9.30 (d, J = 2.4 Hz, 1H), 8.63 (d, J = 2.4 Hz, 1H), 8.16 (s, 1H), 8.11 (s, 1H), 8.00 (s, 1H), 4.32 (s, 3H), 4.10-4.02 (m, 1H), 3.93- 3.86 (m, 1H), 3.81-3.73 (m, 1H), 3.49 (s, 3H), 3.38- 3.31 (m, 1H), 2.29 (s, 3H). | 498.0 | |

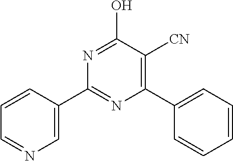 [SMILES: N#Cc1c(O)nc(-c2cccnc2)nc1-c1ccccc1]

Example 314A: 4-hydroxy-6-phenyl-2-(pyridin-3-yl)pyrimidine-5-carbonitrile. A mixture of ethyl (Z)-2-cyano-3-phenyl-prop-2-enoate (5.00 g, 24.8 mmol), pyridine-3-carboxamidine (5.87 g, 37.20 mmol, HCl), potassium carbonate (10.30 g, 74.50 mmol) in ethanol (50 mL) was stirred at 80° C. for 15 hours. The reaction mixture was filtered, the filter cake was collected and dissolved with water, then conc. hydrochloric acid was added, the white solid formed and filtered. The filtered cake was dried to provide the product (5.30 g, 78% yield) as a white solid.

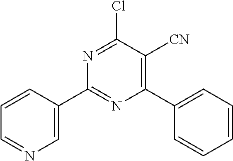 [SMILES: N#Cc1c(Cl)nc(-c2cccnc2)nc1-c1ccccc1]

Example 314B: 4-chloro-6-phenyl-2-(pyridin-3-yl)pyrimidine-5-carbonitrile. To a solution of 4-hydroxy-6-phenyl-2-(3-pyridyl)pyrimidine-5-carbonitrile (5.00 g) in phosphorus oxychloride (50 mL) was stirred at 100° C. for 0.5 hour to provide the product (3.00 g, 98% purity) as a white solid.

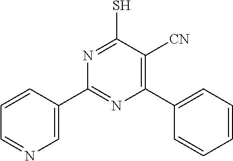 [SMILES: N#Cc1c(S)nc(-c2cccnc2)nc1-c1ccccc1]

Example 314C: 4-mercapto-6-phenyl-2-(pyridin-3-yl)pyrimidine-5-carbonitrile. To a solution of Example 150B (3.00 g) in dimethyl formamide (20 mL) was added sodium sulfide (1.15 g). The mixture was stirred at 80° C. for 2 hours to provide the product (3.50 g, zrude) as a yellow solid.

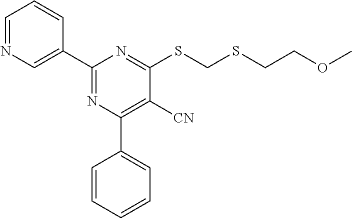 [SMILES: COCCSCSc1nc(-c2cccnc2)nc(-c2ccccc2)c1C#N]

Example 314D: 4-((((2-methoxyethyl)thio)methyl)thio)-6-phenyl-2-(pyridine-3-yl)pyrimidine-5-carbonitrile. Prepared using the General Procedure, step 4 starting from Example 314C (3.00 g) and 1-(chloromethylsulfanyl)-2-methoxy-ethane (3.63 g) to provide the product (1.70 g, 33% yield) as a white solid. ¹H NMR (400 MHz, CDCl3) δ 9.70-9.68 (m, 1H), 8.72-8.71 (m, 2H), 8.06-8.04 (m, 2H), 7.54-7.50 (m, 4H), 4.60 (s, 2H), 3.63 (t, J=6.0 Hz, 2H), 3.32 (s, 3H), 2.88-2.84 (m, 2H).

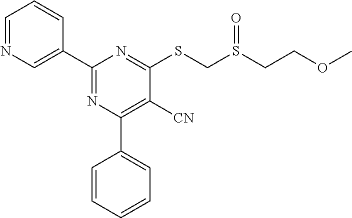 [SMILES: COCCS(=O)CSc1nc(-c2cccnc2)nc(-c2ccccc2)c1C#N]

Example 314E: 4-((((2-methoxyethylsulfinyl)methyl)thio)-6-phenyl-2-(pyridin-3-yl)pyrimidine-5-carbonitrile. Prepared using the General Procedure, step 5 starting from Example 314D (1.70 g, 3.02 mmol) and hydrogen peroxide (769 mg) to provide the product (1.60 g, 91% yield) as a yellow solid.

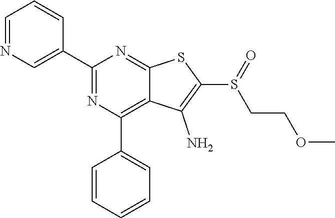 [SMILES: COCCS(=O)c1sc2nc(-c3cccnc3)nc(-c3ccccc3)c2c1N]

EXAMPLE 314: 6-((2-methoxyethyl)sulfinyl)-4-phenyl-2-(pyridin-3-yl)thieno[2,3-d]pyrimidin-5-amine. Prepared using the General Procedure, step 6 starting from Example 314E (1.50 g) to provide the product (600 mg, 56% yield) as a yellow solid.

¹H NMR: (400 MHz, CDCl3) δ 9.76 (d, J=2.0 Hz, 1H), 8.84-8.81 (m, ¹H), 8.73-8.72(m, ¹H), 7.74-7.72 (m, 2H), 7.63-7.61 (m, 3H), 7.43 (m, 1H), 4.85 (s, 2H), 3.88-3.86 (m, 1H), 3.73-3.71 (m, 1H), 3.61-3.60 (m, 1H), 3.40 (s, 3H), 3.29-3.28 (m, 1H). LCMS: (ES⁺) m/z (M+H)⁺=411.0.

The enantiomers were resolved by SFC to give the R isomer (99% ee, [α]=+98.739°) and the S isomer (99% ee, [α]=−94.777°).

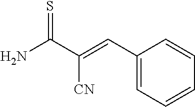 [SMILES: N#C/C(=C\c1ccccc1)C(N)=S]

EXAMPLE 315A: (E)-2-cyano-3-phenylprop-2-enethioamide. To a solution of benzaldehyde (5 g, 47.12 mmol) and 2-cyanothioacetamide (4.72 g, 47.12 mmol) in EtOH (50 mL) was added TEA (476.76 mg, 4.71 mmol). The mixture solution was stirred at 30° C. for 2 hours. The mixture was purified by reversed-phase column (TFA condition) to give the target compound (2.5 g, 28% yield) as a white solid.

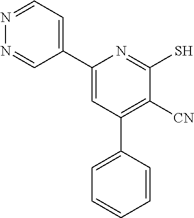 [SMILES: N#Cc1c(-c2ccccc2)cc(-c2ccnnc2)nc1S]

EXAMPLE 315B: 2-mercapto-4-phenyl-6-(pyridazin-4-yl)nicotinonitrile. To a solution of Example 315A (2.2 g, 11.69 mmol) and 1-pyridazin-4-ylethanone (1.43 g, 11.69 mmol) in EtOH (45 mL) was added TEA (118.26 mg, 1.17 mmol, 162.66 μL). The mixture solution was stirred at 80° C. for 16 hours. The solution was concentrated and purified by reversed-phase column (basic condition) to give the target compound (0.5 g, 13% yield) as yellow solid.

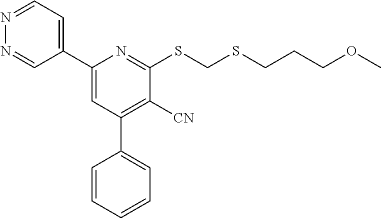 [SMILES: COCCCSCSc1nc(-c2ccnnc2)cc(-c2ccccc2)c1C#N]

EXAMPLE 315C: 2-((((3-methoxypropyl)thio)methyl)thio)-4-phenyl-6-(pyridazin-4-yl)nicotinonitrile. The title compound was prepared by the general procedure starting from Example 315B (0.50 g, 1.72 mmol) and (chloromethyl)(3-methoxypropyl)sulfane (320 mg, 2.07 mmol) to give the target compound (0.30 g, 43% yield) as a brown solid.

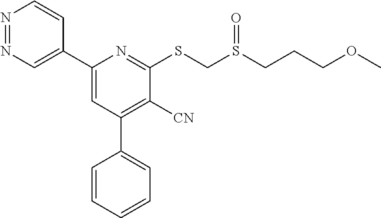 [SMILES: COCCCS(=O)CSc1nc(-c2ccnnc2)cc(-c2ccccc2)c1C#N]

EXAMPLE 315D: 2-((((3-methoxypropyl)sulfinyl)methyl)thio)-4-phenyl-6-(pyridazin-4-yl)nicotinonitrile. The title compound was prepared by the general procedure starting from Example 315C (300 mg, 0.73 mmol) and H₂O₂(167 mg, 1.47 mmol, 30% purity) to give the target compound (300 mg, 96% yield) as a yellow solid.

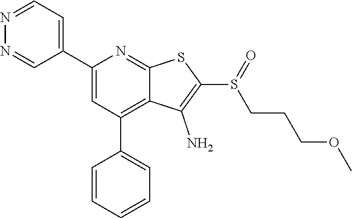 [SMILES: COCCCS(=O)c1sc2nc(-c3ccnnc3)cc(-c3ccccc3)c2c1N]

EXAMPLE 315: 2-((3-methoxypropyl)sulfinyl)-4-phenyl-6-(pyridazin-4-yl)thieno[2,3-b]pyridin-3-amine. The title compound was prepared by the general procedure starting from Example 315D (300 mg, 0.71 mmol) to give the target compound (70 mg, 23% yield) as a yellow solid. LCMS: (ES⁺) m/z (M+H)⁺=425.0 HPLC: tR=1.912 min ¹H NMR (400 MHz, DMSO-d₆) δ 10.03 (d, J=1.2 Hz, 1H), 9.40-9.39 (m, 1H), 8.47-8.45 (m, 1H), 8.17 (s, 1H), 7.65-7.61 (m, 5H), 5.01 (s, 2H), 3.44-3.41 (m, 2H), 3.22 (s, 3H), 3.15 (m, 1H), 3.08-3.05 (m, 1H), 1.88-1.81 (m, 2H).

The following compounds were prepared from known starting materials in a manner similar to that used for EXAMPLE 315. For select compounds, the enantiomers were resolved from the racemate using chiral supercritical fluid chromatography (SFC) and are reported with enantiomeric excess (ee) and/or optical rotation values. In some cases, only the more active R-(+) isomer was prepared/isolated.

| | | 400 MHz NMR (δ; | | |
|---|---|---|---|---|
| Ex. | | CDCl₃ unless otherwise | LCMS | Chiral |
| No. | Structure | noted) | (ES⁺; MH⁺) | info |
| 316 | 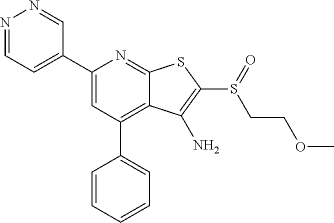 [SMILES: COCCS(=O)c1sc2nc(-c3ccnnc3)cc(-c3ccccc3)c2c1N] | 9.92 (s, 1H), 9.38-9.36 (m, 1H), 8.22-8.20 (m, 1H), 7.71 (s, 1H), 7.62-7.60 (m, 3H), 7.54-7.52 (m, 2H), 4.66 (s, 2H), 3.89-3.86 (m, 1H), 3.74-3.73 (m, 1H), 3.63-3.62 (m, 1H), 3.42 (s, 3H), 3.31-3.30 (m, 1H). | 411.0 | R-isomer: 99.5% ee; [α] = +98.048°; S-isomer: 99.7% ee; [α] = +90.514° |
| 317 | 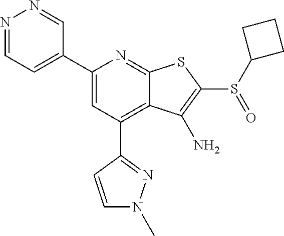 [SMILES: Cn1ccc(-c2cc(-c3ccnnc3)nc3sc(S(=O)C4CCC4)c(N)c23)n1] | DMSO-d6 10.06 (dd, J1 = 2.4, J2 = 1.2 Hz, 1H), 9.42 (dd, J1 = 5.6, J2 = 1.2 Hz, 1H), 8.48-8.46 (m, 1H), 8.36 (s, 1H), 8.05 (d, J = 2.4 Hz, 1H), 7.24 (d, J = 2.4 Hz, 1H), 7.06 2H), 4.03 (s, 3H), 3.96-3.88 (m, 1H), 2.69-2.63 (m, 1H), 2.30-2.09 (m, 3H), 2.06- 1.99 (m, 1H), 1.97-1.92 (m, 1H). | 429.3 | |

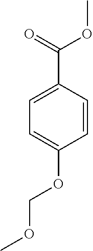

EXAMPLE 318A: methyl 4-(methoxymethoxy)benzoate. To a solution of methyl 4-hydroxybenzoate (20 g, 131.45 mmol) and potassium carbonate (54.50 g, 394.36 mmol) in acetone (200 mL) was stirred at 25° C. for 1 hour. Chloro(methoxy)methane (21.17 g, 262.90 mmol) was then added dropwise at 0° C. The mixture was stirred at 25° C. for 3 hours. The reaction mixture was quenched with aqueous ammonium chloride and the aqueous layer was extracted with ethyl acetate 30 mL (10 mL*3). The combined organic extracts were washed with brine 60 mL (30 mL*2), dried over MgSO₄ and concentration under vacuum to give the target compound (30 g, crude) as a yellow oil.

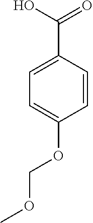

EXAMPLE 318B: 4-(methoxymethoxy)benzoic acid. To a solution of Example 313A (25 g, 127.42 mmol) in water (200 mL) was added sodium hydroxide (6.63 g, 165.65 mmol). The mixture was stirred at 100° C. for 2 hours. The reaction mixture was cooled and poured into acetic acid at 0° C. The crude white product was precipitated, filtered and washed with water. The filter cake was concentrated under high vacuum to give the target compound (22 g, 95% yield) as a white solid. ¹H NMR (400 MHz, CDCl3) δ 8.01-7.99 (m, 2H), 7.03-7.01 (m, 2H), 5.18 (s, 2H), 3.42 (s, 3H).

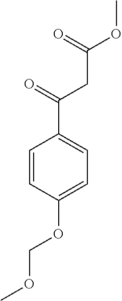

EXAMPLE 318C: ethyl-3-(4-(methoxymethoxy)phenyl)-3-oxopropanoate. To a solution of di(imidazol-1-yl)methanone (20.56 g, 126.80 mmol) and Example 313B (22 g, 120.76 mmol) in THF (200 mL) was stirred at 60° C. for 1 hour, After cooled to 20° C., the mixture was added with potassium 3-ethoxy-3-oxo-propanoate (20.55 g, 120.76 mmol) and magnesium chloride (13.80 g, 144.92 mmol). The mixture was stirred at 60° C. for 1 hour. The mixture was concentrated in vacuum. The residue was partitioned between water (30 mL) and dichloromethane (60 mL). The organic layer was separated, and the aqueous layer was further extracted with dichloromethane 90 mL (3×30 mL). The combined organic layers were washed with water, brine, dried over sodium sulfate, filtered, and concentrated in vacuum to give a residue. The residue was purified by reversed-phase flash (TFA condition) to give the target compound (16 g, 52% yield) as a yellow oil.

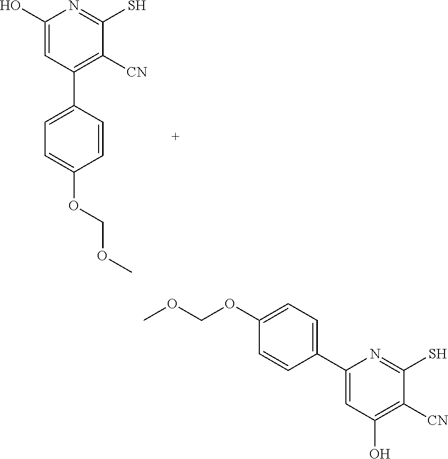 [SMILES: COCOc1ccc(-c2cc(O)nc(S)c2C#N)cc1 ; COCOc1ccc(-c2cc(O)c(C#N)c(S)n2)cc1]

EXAMPLE 318D and EXAMPLE 319A: ethyl-3-(4-(methoxymethoxy)phenyl)-3-oxopropanoate and 4-hydroxy-2-mercapto-6-(4-(methoxymethoxy)phenyl) nicotinonitrile. To a solution of Example 313C (15 g, 59.46 mmol) and 2-cyanothioacetamide (11.91 g, 118.92 mmol) in N,N-Dimethylformamide (30 mL) was added potassium hydroxide (4.34 g, 77.30 mmol). The mixture was stirred at 60° C. for 6 hours. The mixture was purified by Prep-HPLC (basic condition) to give a mixture Example 318D and Example 319A (16 g, 52% yield) as a black oil.

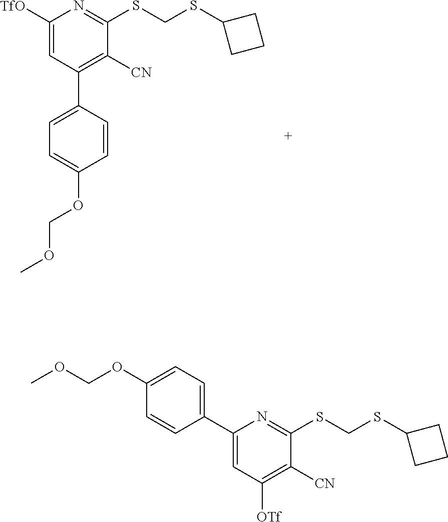 [SMILES: COCOc1ccc(-c2cc(OS(=O)(=O)C(F)(F)F)nc(SCSC3CCC3)c2C#N)cc1 ; COCOc1ccc(-c2cc(OS(=O)(=O)C(F)(F)F)c(C#N)c(SCSC3CCC3)n2)cc1]

EXAMPLE 318E and EXAMPLE 319B: 5-cyano-6-(((cyclobutylthio)methyl)thio)-4-(4-(methoxymethoxy)phenyl)pyridin-2-yl trifluoromethanesulfonate and 3-cyano-2-(((cyclobutylthio)methyl)thio)-6-(4-(methoxymethoxy)phenyl)pyridin-4-yl trifluoromethanesulfonate. To a solution of Example 318D and Example 319A (1.7 g, 4.38 mmol) and 1,1,1-trifluoro-N-phenyl-N-(trifluoromethylsulfonyl)methanesulfonamide (2.34 g, 6.56 mmol) in THF (50 mL) was added potassium 2-methylpropan-2-olate (737 mg, 6.56 mmol). The mixture was stirred at 25° C. for 6 hours to give a mixture of Example 318E and Example 319B (2.5 g, crude) as a yellow solid.

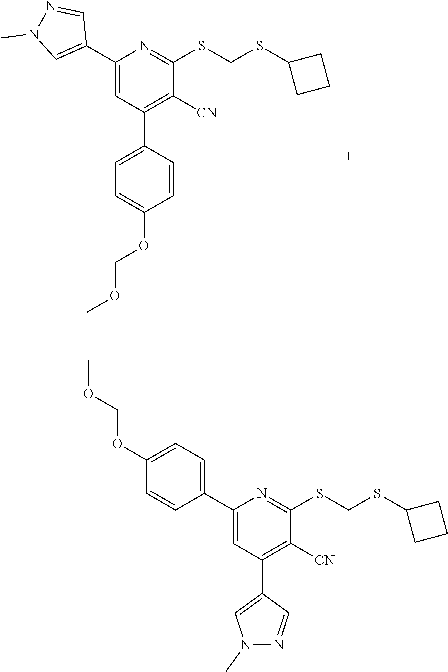 [SMILES: COCOc1ccc(-c2cc(-c3cnn(C)c3)nc(SCSC3CCC3)c2C#N)cc1 ; COCOc1ccc(-c2cc(-c3cnn(C)c3)c(C#N)c(SCSC3CCC3)n2)cc1]

EXAMPLE 318F and EXAMPLE 319C: 2-(((cyclobutylthio)methyl)thio)-4-(4-(methoxymethoxy)phenyl)-6-(1-methyl-1H-pyrazol-4-yl)nicotinonitrile and 2-(((cyclobutylthio)methyl)thio)-6-(4-(methoxymethoxy)phenyl)-4-(1-methyl-1H-pyrazol-4-yl)nicotinonitrile. To a solution of Example 318E and Example 319B (2.5 g, 4.80 mmol) and (1-methylpyrazol-4-yl)boronic acid (907 mg, 7.20 mmol) in tetrahydrofuran (40 mL) and water (20 mL) was added Pd(dppf)Cl₂·CH₂Cl₂ (196 mg, 240 umol) and sodium carbonate (1.02 g, 9.60 mmol). The mixture was stirred at 80° C. for 2 hours. The reaction mixture was concentrated, the residue was purified by column chromatography (SiO₂, Petroleum ether: Ethyl acetate=4:1 to 2:1), then the crude product was purified by Prep-HPLC (column: Waters Xbridge 150*255 u; mobile phase: [water (10 mm NH₄HCO₃)-ACN]; B %: 60%-90%, 14 min) to give Example 318F (400 mg, 37% yield) as a yellow solid and Example 314C (260 mg, 24% yield) as a yellow solid.

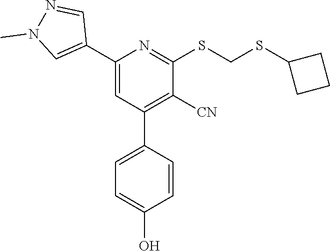 [SMILES: Cn1cc(-c2cc(-c3ccc(O)cc3)c(C#N)c(SCSC3CCC3)n2)cn1]

EXAMPLE 318G: 2-(((cyclobutylthio)methyl)thio)-4-(4-(methoxymethoxy)phenyl)-6-(1-methyl-1H-pyrazol-4-yl)nicotinonitrile. To a solution of Example 313F (260 mg, 574 umol) in CH₂Cl₂ (10 mL) was added CF₃COOH (2.0 g, 17.23 mmol). The mixture was stirred at 25° C. for 2 hours. The pH of the mixture was adjusted to 7 by saturated sodium bicarbonates, and extracted with DCM 60 mL (20 mL*3), the organic phase was concentrated to give compound Example 318G (300 mg, crude) as a white solid.

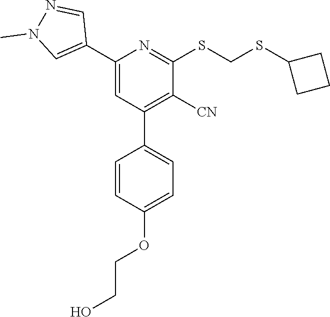 [SMILES: Cn1cc(-c2cc(-c3ccc(OCCO)cc3)c(C#N)c(SCSC3CCC3)n2)cn1]

EXAMPLE 318H: 2-(((cyclobutylthio)methyl)thio)-4-(4-(2-hydroxyethoxy) phenyl)-6-(1-methyl-1H-pyrazol-4-yl)nicotinonitrile. To a solution of Example 318G (300 mg, 734 umol) and 2-bromoethanol (459 mg, 3.67 mmol) in DMF (2 mL) was added K₂CO₃ (203 mg, 1.47 mmol). The mixture was stirred at 50° C. for 6 hours. The mixture was filtered and concentrated, the crude residue was purified by Prep-HPLC (column: Waters Xbridge 150*255 u; mobile phase: [water (10 mM NH₄HCO₃)-ACN]; B %: 44%-74%, 10 min) to give Example 318H (150 mg, 331 umol, 45% yield) as a white solid.

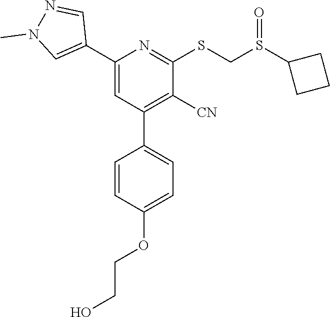 [SMILES: Cn1cc(-c2cc(-c3ccc(OCCO)cc3)c(C#N)c(SCS(=O)C3CCC3)n2)cn1]

EXAMPLE 3181: 2-(((cyclobutylsulfinyl)methyl)thio)-4-(4-(2-hydroxyeth oxy)phenyl)-6-(1-methyl-1H-pyrazol-4-yl)nicotinonitrile. The target compound was prepared by the general procedure starting from Example 318H (150 mg, 331 umol) and hydrogen peroxide (537 mg, 8.95 mmol, 512 uL) to give the target compound (155 mg, 99% yield) as white solid.

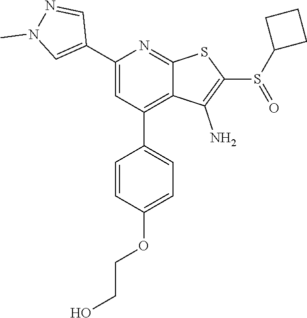 [SMILES: Cn1cc(-c2cc(-c3ccc(OCCO)cc3)c3c(N)c(S(=O)C4CCC4)sc3n2)cn1]

EXAMPLE 318: 2-(4-(3-amino-2-(cyclobutylsulfinyl)-6-(1-methyl-1H-pyrazol-4-yl)thieno[2,3-b]pyridin-4-yl)phenoxy)ethanol. The target compound was prepared by the general procedure starting from Example 3131 (150 mg, 320.11 umol) to give Example 313 (96 mg, 63% yield) as a yellow solid. LCMS: (ES⁺) m/z (M+H)⁺=469.2. HPLC: tR=1.903 min. ¹H NMR (400 MHz, CDCl3) δ 8.02-7.98 (m, 2H), 7.39 (d, J=7.6 Hz, 2H), 7.22 (s, 1H), 7.06 (d, J=8.8 Hz, 2H), 4.56 (s, 2H), 4.19-4.16 (m, 2H), 4.04-4.02 (m, 2H), 3.97 (s, 3H), 3.94-3.90 (m, 1H), 2.83-2.73 (m, 1H), 2.39-2.30 (m, 3H), 2.27-2.18 (m, 1H), 2.11-2.02 (m, 2H).

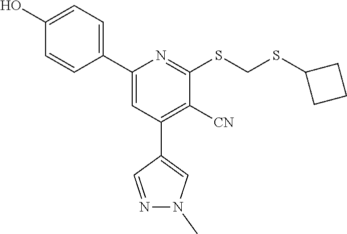 [SMILES: Cn1cc(-c2cc(-c3ccc(O)cc3)nc(SCSC3CCC3)c2C#N)cn1]

EXAMPLE 319D: 2-(((cyclobutylthio)methyl)thio)-6-(4-hydroxyphenyl)-4-(1-methyl-1H-pyrazol-4-yl)nicotinonitrile. The target compound was prepared by the procedure used for Example 313G starting from Example 314C (2-(cyclobutylsulfanylmethylsulfanyl)-6-[4-(methoxymethoxy)phenyl]-4-(1-methylpyrazol-4-yl)pyridine-3-carbonitrile) (400 mg, 884 umol). The pH of the mixture was adjusted to 7 by saturated sodium bicarbonate, and extracted with DCM 60 mL (20 mL*3), the organic phase was concentrated to give the target compound (300 mg, 83% yield) as a white solid.

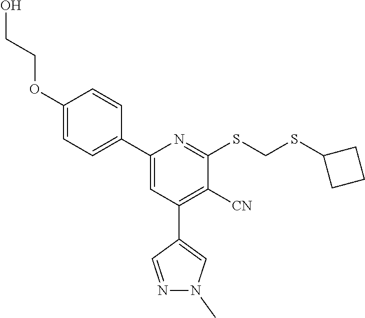 [SMILES: Cn1cc(-c2cc(-c3ccc(OCCO)cc3)nc(SCSC3CCC3)c2C#N)cn1]

EXAMPLE 319E: 2-(((cyclobutylthio)methyl)thio)-6-(4-(2-hydroxyethoxy)phenyl)-4-(1-methyl-1H-pyrazol-4-yl)nicotinonitrile. The title compound was prepared by the procedure used for Example 318H starting from Example 319D (300 mg, 734 umol) and 2-bromoethanol (275 mg, 2.20 mmol, 156 uL) to give the target compound (150 mg, 331 umol, 45% yield) as a white solid.

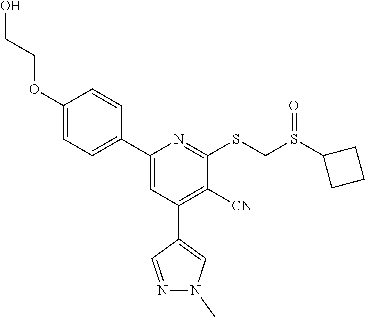 [SMILES: Cn1cc(-c2cc(-c3ccc(OCCO)cc3)nc(SCS(=O)C3CCC3)c2C#N)cn1]

EXAMPLE 319F: 2-(((cyclobutylsulfinyl)methyl)thio)-6-(4-(2-hydroxyethoxy)phenyl)-4-(1-methyl-1H-pyrazol-4-yl)nicotinonitrile. The title compound was prepared by the general procedure starting from Example 319E (150 mg, 331 umol) and hydrogen peroxide (75 mg, 663 umol, 64 μL, 30% purity) to give EXAMPLE 319F (155 mg, 99% yield) as white solid.

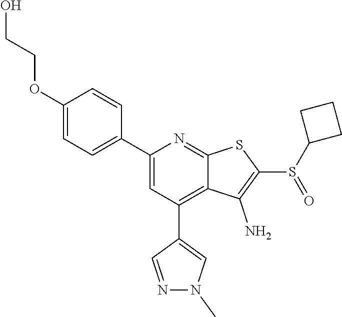 [SMILES: Cn1cc(-c2cc(-c3ccc(OCCO)cc3)nc3sc(S(=O)C4CCC4)c(N)c23)cn1]

EXAMPLE 319: 2-(4-(3-amino-2-(cyclobutylsulfinyl)-6-(1-methyl-1H-pyrazol-4-yl)thieno[2,3-b]pyridin-4-yl)phenoxy)ethanol. The title compound was prepared by the general procedure starting from Example 314F (150 mg, 320 umol) to give compound EXAMPLE 319 (96 mg, 63% yield) as yellow solid. LCMS: (ES⁺) m/z (M+H)⁺=469.2. HPLC: tR=1.894 min. ¹H NMR (400 MHz, CDCl3) δ 8.01 (d, J=8.8 Hz, 2H), 7.73 (s, 1H), 7.67 (s, 1H), 7.43 (s, 1H), 7.00 (d, J=8.8 Hz, 2H), 4.88 (s, 2H), 4.16-4.14 (m, 2H), 4.04 (s, 3H), 4.02-3.99 (m, 2H), 3.97-3.93 (m, 1H), 2.86-2.76 (m, 1H), 2.43-2.33 (m, 2H), 2.28-2.19 (m, 2H), 2.13-2.04 (m, 2H).

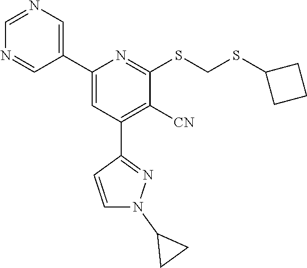 [SMILES: N#Cc1c(-c2ccn(C3CC3)n2)cc(-c2cncnc2)nc1SCSC1CCC1]

EXAMPLE 320A: 2-(((cyclobutylthio)methyl)thio)-4-(1-cyclopropyl-1H-pyrazol-3-yl)-6-(pyrimidin-5-yl)nicotinonitrile. To a solution of 2-(cyclobutylsulfanylmethylsulfanyl)-4-(1H-pyrazol-3-yl)-6-pyrimidin-5-yl-pyridine-3-carbonitrile (500 mg, 1.31 mmol) and potassium cyclopropyl(trifluoro)borate (389 mg, 2.63 mmol) in DCE (5 mL) was added 2-(2-pyridyl)pyridine (205 mg, 1.31 mmol), Na₂CO₃ (279 mg, 2.63 mmol) and Cu(OAc)₂ (238.68 mg, 1.31 mmol, 1 eq). The mixture was stirred at 70° C. for 20 hours. The mixture was concentrated under high vacuum, the residue was purified by Prep-HPLC (column: Phenomenex Luna C18 250*50 mm*10 um; mobile phase: [water (0.075% TFA)-ACN]; B %: 58%-88%, 9 min) to give the target compound (280 mg, 51% yield) as a white solid. ¹H NMR (400 MHz, CHCl₃) δ 9.46 (s, 2H), 9.32 (s, 1H), 8.15 (s, 1H), 7.61 (d, J=2.4 Hz, 1H), 7.17 (d, J=2.4 Hz, 1H), 4.45 (s, 2H), 3.79-3.70 (m, 2H), 2.44-2.37 (m, 2H), 2.15-2.05 (m, 2H), 2.04-1.92 (m, 2H), 1.28-1.24 (m, 2H), 1.16-1.11 (m, 2H).

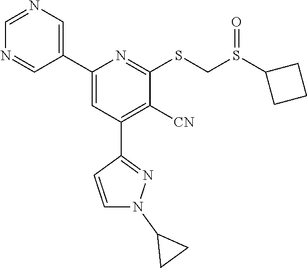 [SMILES: N#Cc1c(-c2ccn(C3CC3)n2)cc(-c2cncnc2)nc1SCS(=O)C1CCC1]

EXAMPLE 320B: 2-(((cyclobutylsulfinyl)methyl)thio)-4-(1-cyclopropyl-1H-pyrazol-3-yl)-6-(pyrimidin-5-yl)nicotinonitrile. The title compound was prepared by the general procedure starting from Example 315A (270 mg, 642 umol) and hydrogen peroxide (1.04 g, 17.33 mmol, 991 uL) to give the title compound (270 mg, 619 umol, 96% yield) as a white solid.

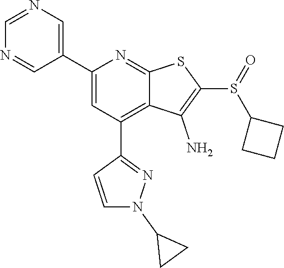 [SMILES: Nc1c(S(=O)C2CCC2)sc2nc(-c3cncnc3)cc(-c3ccn(C4CC4)n3)c12]

EXAMPLE 320: 2-(cyclobutylsulfinyl)-4-(1-cyclopropyl-1H-pyrazol-3-yl)-6-(pyrimidin-5-yl)thieno[2,3-b]pyridin-3-amine. The title compound was prepared by the general procedure starting from Example 315B (270 mg, 619 umol) to give compound EXAMPLE 315 (236 mg, 86% yield) as a yellow solid. LCMS: (ES⁺) m/z (M+H)⁺=437.2. HPLC: tR=2.165 min. ¹H NMR (400 MHz, DMSO-d₆) δ 9.58 (s, 2H), 9.30 (s, 1H), 8.26 (s, 1H), 8.15 (d, J=2.4 Hz, 1H), 7.20 (d, J=2.4 Hz, 1H), 7.06 (s, 2H), 3.96-3.86 (m, 2H), 2.69-2.60 (m, 1H), 2.24-2.12 (m, 3H), 2.07-1.98 (m, 1H), 1.97-1.90 (m, 1H), 1.15-1.13 (m, 2H), 1.11-1.08 (m, 2H).

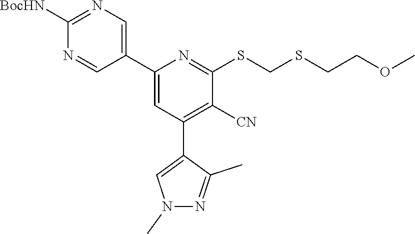 [SMILES: COCCSCSc1nc(-c2cnc(NC(=O)OC(C)(C)C)nc2)cc(-c2cn(C)nc2C)c1C#N]

EXAMPLE 321A: tert-butyl (5-(5-cyano-4-(1,3-dimethyl-1H-pyrazol-4-yl)-6-((((2-methoxyethyl)thio)methyl)thio)pyridin-2-yl)pyrimidin-2-yl)carbamate. The title compound was prepared by the general procedure starting from tert-butyl (5-(5-cyano-4-(1,3-dimethyl-1H-pyrazol-4-yl)-6-mercaptopyridin-2-yl)pyrimidin-2-yl)carbamate (0.5 g, 1.18 mmol) and (bromomethyl)(2-methoxyethyl)sulfane (435 mg, 2.36 mmol) to provide the title compound (410 mg, 778 umol, 50% yield) as a yellow solid.

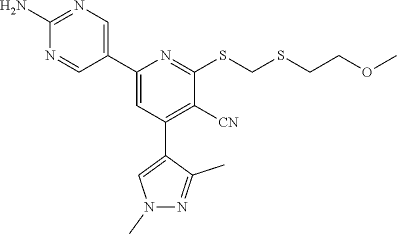 [SMILES: COCCSCSc1nc(-c2cnc(N)nc2)cc(-c2cn(C)nc2C)c1C#N]

EXAMPLE 321B: 6-(2-aminopyrimidin-5-yl)-4-(1,3-dimethyl-1H-pyrazol-4-yl)-2-((((2-methoxyethyl)thio)methyl)thio)nicotinonitrile. To a solution of EXAMPLE 321A (360 mg, 682 umol) in dichloromethane (3 mL) was added trifluoroacetic acid (462 mg, 4.05 mmol, 0.3 mL). The mixture was stirred at 25° C. for 1 hour. The reaction mixture was quenched by addition saturated sodium carbonate (50 mL), and then diluted with water (50 mL) and extracted with dichloromethane (150 mL). The combined organic layers were concentrated and lyophilized to give the title compound (0.3 g, crude) as brown solid.

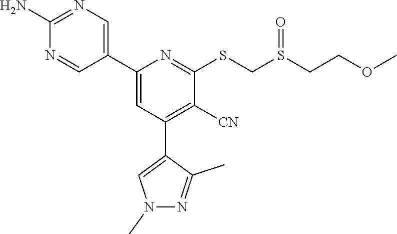 [SMILES: COCCS(=O)CSc1nc(-c2cnc(N)nc2)cc(-c2cn(C)nc2C)c1C#N]

EXAMPLE 321C: 6-(2-aminopyrimidin-5-yl)-4-(1,3-dimethyl-1H-pyrazol-4-yl)-2-((((2-methoxyethyl)sulfinyl)methyl)thio)nicotinonitrile. To a solution of EXAMPLE 321B (280 mg, crude) in chloroform (10 mL) was added 3-chlorobenzoperoxoic acid (113 mg, 655 umol). The mixture was stirred at 25° C. for 1 hour. The reaction mixture was quenched by addition of sodium sulfite 50 ml at 0° C., and then diluted with sodium bicarbonate 50 mL and extracted with ethyl acetate 200 mL. The combined organic layers were washed with saturated sodium chloride 20 mL, dried over sodium sulfate, filtered and concentrated under reduced pressure to give a residue. The residue was purified by reversed-phase HPLC (0.1% NH₃·H₂O) to give the target compound (150 mg, 338 umol, 52% yield) as brown solid. ¹H NMR (400 MHz, CDCl3) δ 8.99 (s, 2H), 7.88 (s, 1H), 7.40 (s, 1H), 5.48 (s, 2H), 4.77-4.63 (m, 2H), 4.01-3.95 (m, 1H), 3.94 (s, 3H), 3.87-3.80 (m, 1H), 3.41 (s, 3H), 3.23-3.13 (m, 1H), 3.09-3.01 (m, 1H), 2.43 (s, 3H).

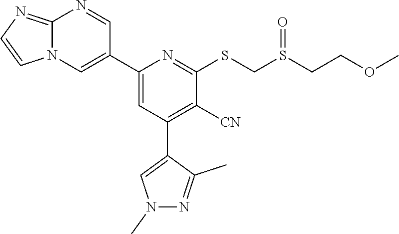 [SMILES: COCCS(=O)CSc1nc(-c2cnc3nccn3c2)cc(-c2cn(C)nc2C)c1C#N]

EXAMPLE 321D: To a solution of EXAMPLE 321C (140 mg, 316 umol) in ethyl alcohol was added 2-chloroacetaldehyde (310 mg, 1.58 mmol, 254 uL). The mixture was stirred at 80° C. for 3 hours. The solution was concentrated and purified by Prep-HPLC to give the title compound (110 mg, 235 umol, 75% yield) as a yellow solid.

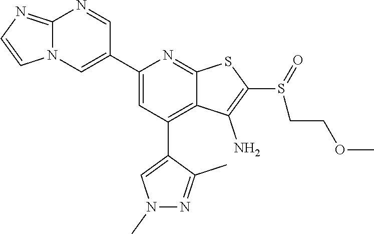 [SMILES: COCCS(=O)c1sc2nc(-c3cnc4nccn4c3)cc(-c3cn(C)nc3C)c2c1N]

EXAMPLE 321: 4-(1,3-dimethyl-1H-pyrazol-4-yl)-6-(imidazo[1,2-a]pyrimidin-6-yl)-2-((2-methoxyethyl)sulfinyl)thieno[2,3-b]pyridin-3-amine. The title compound was prepared by the general procedure starting from EXAMPLE 321D (50 mg, 107 umol) and potassium hydroxide solution (40 mg, 214 umol, 10% purity) to get EXAMPLE 321 (4 mg, 8 umol, 7% yield, 98.08% purity) and a second, less-pure sample of EXAMPLE 321 (6 mg, 11 umol, 10% yield, 94.16% purity), both as yellow solids. LCMS: (ES⁺) m/z (M+H)⁺=468.1. HPLC: tR=1.487 min. ¹HNMR (400 MHz, CDCl3) δ 9.36 (d, J=2.4 Hz, 1H), 9.16 (d, J=2.4 Hz, 1H), 7.90 (d, J=1.2 Hz, 1H), 7.68 (d, J=1.2 Hz, 1H), 7.53 (s, 1H), 7.52 (s, 1H), 4.86 (s, 2H), 3.99 (s, 3H), 3.93-3.86 (m, 1H), 3.75-3.70 (m, 1H), 3.65-3.59 (m, 1H), 3.42 (s, 3H), 3.29 (m, 1H), 2.26 (s, 3H).

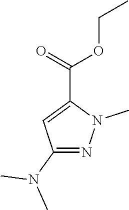

EXAMPLE 322A: ethyl 3-(dimethylamino)-1-methyl-1H-pyrazole-5-carboxylate. To a solution of ethyl 3-amino-1-methyl-1H-pyrazole-5-carboxylate ([89088-57-3], 10.6 g, 62.7 mmol) in MeOH (200 mL) and THF (200 mL) was added NaBH₃CN (15.8 g, 251 mmol) and HCHO (9.41 g, 313 mmol) at 25° C. Then the mixture was adjusted to pH=5 by addition of acetic acid and stirred at 25° C. for 12 hours. The reaction mixture was quenched by addition of Na₂CO₃ to adjust the pH=10. The reaction mixture was concentrated under reduced pressure to give a residue. The residue was diluted with H₂O 200 mL and extracted with DCM 900 mL (300 mL*3). The combined organic layers were dried over Na2SO₄, filtered and concentrated under reduced pressure to give a residue. The residue was purified by column chromatography (SiO₂, Petroleum ether: Ethyl acetate=50:1 to 20:1) and reversed-phase HPLC (0.1% FA condition). The title compound (1.90 g, 15%) was obtained as yellow oil. ¹H NMR (400 MHz, CDCl3) δ 6.16 (s, 1H), 4.33 (q, J=7.2 Hz, 2H), 4.04 (s, 3H), 2.86 (s, 6H), 1.38 (t, J=7.2 Hz, 3H).

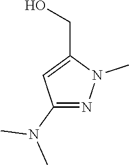

EXAMPLE 322B: (3-(dimethylamino)-1-methyl-1H-pyrazol-5-yl)methanol. To a solution of EXAMPLE 322A (1.40 g, 7.10 mmol) in DCM (30 mL) was added DIBAL-H (1 M, 11.4 mL) at −70° C. Then the mixture was stirred at −70° C. for 1 hour. The reaction mixture was quenched by addition H₂O 30 mL at −60° C. The solution was concentrated under reduced pressure to give a residue. The residue was purified by column chromatography (SiO₂, Petroleum ether: Ethyl acetate=5:1 to 1:3). The title compound (1.00 g, 91%) was obtained as a yellow oil. ¹H NMR (400 MHz, CDCl3) δ 5.53 (s, 1H), 4.54 (s, 2H), 3.70 (s, 3H), 2.82 (s, 6H).

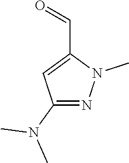

EXAMPLE 322C: 3-(dimethylamino)-1-methyl-1H-pyrazole-5-carbaldehyde. To a solution of EXAMPLE 322B (1.60 g, 10.3 mmol) in DCM (20 mL) was added MnO₂ (9.77 g, 112 mmol). The mixture was stirred at 25° C. for 12 hours. The reaction mixture was filtered. The filtrate was concentrated to give a residue. The residue was purified by column chromatography (SiO₂, Petroleum ether: Ethyl acetate=10:1 to 1:1) to give the title compound (800 mg, 51%) as yellow oil. ¹H NMR (400 MHz, CDCl3) δ 9.67 (s, 1H), 6.08 (s, 1H), 3.96 (s, 3H), 2.80 (s, 6H).

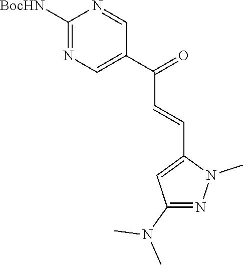 [SMILES: CN(C)c1cc(/C=C/C(=O)c2cnc(NC(=O)OC(C)(C)C)nc2)n(C)n1]

EXAMPLE 322D: (E)-tert-butyl(5-(3-(3-(dimethylamino)-1-methyl-1H-pyrazol-5-yl)acryloyl)pyrimidin-2-yl)carbamate. The title compound was prepared by General Procedure A starting from EXAMPLE 322C (700 mg, 4.57 mmol) and tert-butyl N-(5-acetyl pyrimidin-2-yl)-N-tert-butoxycarbonyl-carbamate (1.85 g, 5.48 mmol) to give the title compound (1.20 g, crude) as a red solid.

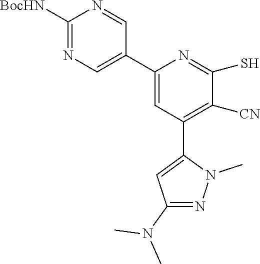 [SMILES: CN(C)c1cc(-c2cc(-c3cnc(NC(=O)OC(C)(C)C)nc3)nc(S)c2C#N)n(C)n1]

EXAMPLE 322E: tert-butyl (5-(5-cyano-4-(3-(dimethylamino)-1-methyl-1H-pyrazol-5-yl)-6-mercaptopyridin-2-yl)pyrimidin-2-yl)carbamate. The title compound was prepared by General Procedure A starting from EXAMPLE 322D (600 mg, 1.61 mmol) and 2-cyanoethanethioamide (242 mg, 2.42 mmol) to give the title compound (700 mg, crude) as yellow solid.

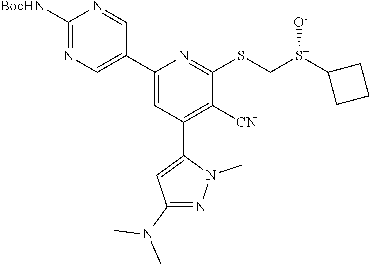 [SMILES: CN(C)c1cc(-c2cc(-c3cnc(NC(=O)OC(C)(C)C)nc3)nc(SC[S@+]([O-])C3CCC3)c2C#N)n(C)n1]

EXAMPLE 322F: (R)-tert-butyl(5-(5-cyano-6-(((cyclobutylsulfinyl)methyl) thio)-4-(3-(dimethylamino)-1-methyl-1H-pyrazol-5-yl)pyridin-2-yl)pyrimidin-2-yl)carbamate. The title compound was prepared by the general procedure starting from EXAMPLE 322E (700 mg, 1.55 mmol) and (R)-((bromo methyl)sulfinyl)cyclobutane (213 mg, 1.08 mmol) to give the title compound (300 mg, 34%) as yellow solid.

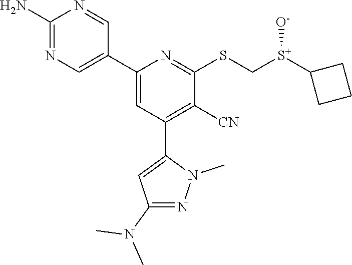 [SMILES: CN(C)c1cc(-c2cc(-c3cnc(N)nc3)nc(SC[S@+]([O-])C3CCC3)c2C#N)n(C)n1]

EXAMPLE 322G: (R)-6-(2-aminopyrimidin-5-yl)-2-(((cyclobutylsulfinyl)meth yl)thio)-4-(3-(dimethylamino)-1-methyl-1H-pyrazol-5-yl)nicotinonitrile. To a solution of EXAMPLE 322F (300 mg, 528 umol) in DCM (3 mL) was added TFA (301 mg, 2.64 mmol, 195 uL). The mixture was stirred at 25° C. for 2 hours. The reaction mixture was concentrated under reduced pressure to give a residue. The crude product was purified by reversed-phase HPLC (0.1% TFA condition). The title compound (220 mg, 72%, TFA) was obtained as a yellow solid. ¹H NMR (400 MHz, CDCl3) δ 9.01 (s, 2H), 7.33 (s, 1H), 6.40 (s, 2H), 5.87 (s, 1H), 4.50 (d, J=13.2 Hz, 1H), 4.06 (d, J=13.2 Hz, 1H), 3.68 (s, 3H), 2.84 (s, 6H), 2.77-2.65 (m, 1H), 2.46-2.33 (m, 1H), 2.23-2.02 (m, 3H), 2.00-1.89 (m, 2H).

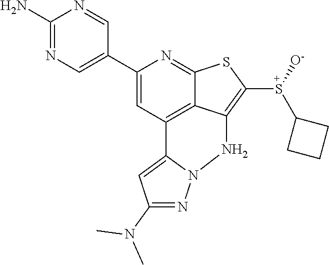

EXAMPLE 322: (R)-6-(2-aminopyrimidin-5-yl)-2-(cyclobutylsulfinyl)-4-(3-(dimethylamino)-1-methyl-1H-pyrazol-5-yl)thieno[2,3-b]pyridin-3-amine. The title compound was prepared by the general procedure starting from EXAMPLE 322G (200 mg, 343 umol) to give EXAMPLE 322 (114.4 mg, 71% yield, 99.2% purity) as a yellow solid. Optical Rotation determination showed the Specific Rotation was +105.454°; LCMS: (ES⁺) m/z (M+H)⁺=469.1. HPLC: tR=1.632 min. ¹H NMR (400 MHz, CDCl3) δ 9.04 (s, 2H), 7.47 (s, 1H), 5.85-5.81 (m, 1H), 5.34 (s, 2H), 4.84-4.67 (m, 2H), 4.02-3.90 (m, 1H), 3.59-3.55 (m, 3H), 2.93 (s, 6H), 2.90-2.78 (m, 1H), 2.45-2.33 (m, 2H), 2.32-2.21 (m, 1H), 2.17-2.03 (m, 2H).

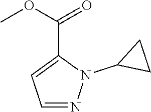

EXAMPLE 323A: methyl 1-cyclopropyl-1H-pyrazole-5-carboxylate. To a solution of methyl 1H-pyrazole-3-carboxylate (24 g, 190 mmol) and cyclopropylboronic acid (32.7 g, 380 mmol) and Na2CO₃ (40.3 g, 380 mmol) in DCE (300 mL) was added 2-(2-pyridyl)pyridine (29.7 g, 190 mmol) and Cu(OAc)₂ (34.6 g, 190 mmol). The mixture was stirred at 70° C. for 48 hours under 02 (15 psi). The mixture was filtrate and concentrated, the crude product was purified by column chromatography (SiO₂, Petroleum ether:Ethyl acetate=10:1 to 2:1) to give the title compound (10 g, 32% yield) as a yellow oil. ¹H NMR (400 MHz, CDCl3) δ 7.40 (d, J=2.0 Hz, 1H), 6.83 (d, J=2.0 Hz, 1H), 4.33-4.27 (m, 1H), 3.90 (s, 3H), 1.28-1.26 (m, 2H), 1.08-1.03 (m, 2H).

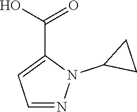 [SMILES: O=C(O)c1ccnn1C1CC1]

EXAMPLE 323B: 1-cyclopropyl-1H-pyrazole-5-carboxylic acid. To a solution of EXAMPLE 323A (7 g, 42.1 mmol) in H₂O (10 mL) was added NaOH (2.19 g, 54.8 mmol, 1.3 eq). The mixture was stirred at 100° C. for 2 hours. The pH of the mixture was adjust to 7 by acetic acid and then was concentrated, the crude product was purified by Prep-HPLC (TFA condition) to give the title compound (11 g, crude) as a white solid.

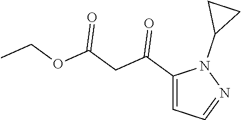 [SMILES: CCOC(=O)CC(=O)c1ccnn1C1CC1]

EXAMPLE 323C: ethyl 3-(1-cyclopropyl-1H-pyrazol-5-yl)-3-oxopropanoate. The title compound was prepared by the procedure used for Example 313C starting from EXAMPLE 323B (10 g, 65.7 mmol) and potassium 3-ethoxy-3-oxo-propanoate (13.4 g, 78.9 mmol) to give EXAMPLE 318C (10 g, 69% yield) as a yellow oil. ¹H NMR (400 MHz, CDCl3) δ 7.41 (d, J=2.0 Hz, 1H), 6.82 (d, J=2.0 Hz, 1H), 4.32-4.29 (m, 1H), 4.24-4.18 (m, 2H), 3.85 (s, 2H), 1.26 (t, J=7.2 Hz, 3H), 1.23-1.21 (m, 2H), 1.06-1.03 (m, 2H).

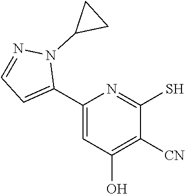 [SMILES: N#Cc1c(O)cc(-c2ccnn2C2CC2)nc1S]

EXAMPLE 323D: 6-(1-cyclopropyl-1H-pyrazol-5-yl)-4-hydroxy-2-mercaptonicotinonitrile. The title compound was prepared by the general procedure starting from EXAMPLE 323C (5 g, 22.5 mmol) and 2-cyanoethanethioamide (4.51 g, 45 mmol) to give the title compound (5 g, 19.4 mmol, 86% yield) as a yellow solid.

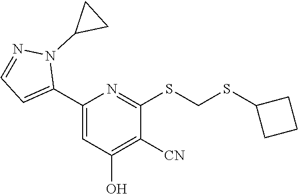 [SMILES: N#Cc1c(O)cc(-c2ccnn2C2CC2)nc1SCSC1CCC1]

EXAMPLE 323E: 2-(((cyclobutylthio)methyl)thio)-6-(1-cyclopropyl-1H-pyrazol-5-yl)-4-hydroxynicotinonitrile. The title compound was prepared by the general procedure starting from EXAMPLE 323D (4.50 g, 17.4 mmol) and chloromethylsulfanylcyclobutane (2.38 g, 17.4 mmol) to give the crude title compound (2.5 g, 40% yield) as a brown oil.

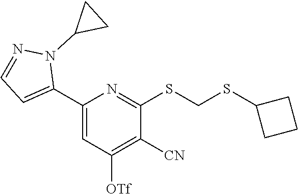 [SMILES: N#Cc1c(OS(=O)(=O)C(F)(F)F)cc(-c2ccnn2C2CC2)nc1SCSC1CCC1]

EXAMPLE 323F: 3-cyano-2-(((cyclobutylthio)methyl)thio)-6-(1-cyclopropyl-1H-pyrazol-5-yl)pyridin-4-yltrifluoromethanesulfonate. The title compound was prepared by the general procedure starting from EXAMPLE 323E (2.50 g, 6.97 mmol) and 1,1,1-trifluoro-N-phenyl-N-((trifluoromethyl)sulfonyl)methanesulfonamide (4.98 g, 13.9 mmol) to give the title compound (1.6 g, 47% yield) as a yellow solid.

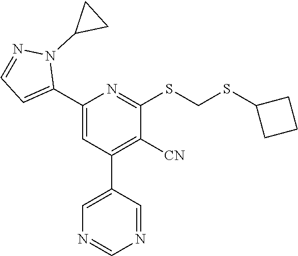 [SMILES: N#Cc1c(-c2cncnc2)cc(-c2ccnn2C2CC2)nc1SCSC1CCC1]

EXAMPLE 323G: 2-((((3-methoxypropyl)thio)methyl)thio)-4-(pyridazin-4-yl)-6-(pyrimidin-5-yl)nicotinonitrile. The title compound was prepared by the general procedure starting from EXAMPLE 323F (600 mg, 1.22 mmol) and pyrimidin-5-ylboronic acid (181 mg, 1.47 mmol) to give the title compound (320 mg, 761 umol) as a white solid. ¹H NMR (400 MHz, CDCl3) δ 9.40 (s, 1H), 9.02 (s, 2H), 7.51 (d, J=2.0 Hz, 1H), 7.46 (s, 1H), 6.75 (d, J=2.0 Hz, 1H), 4.46 (s, 2H), 4.29-4.25 (m, 1H), 3.74-3.72 (m, 1H), 2.42-2.39 (m, 2H), 2.08-2.05 (m, 4H), 1.28-1.26 (m, 2H), 1.10-1.08 (m, 2H).

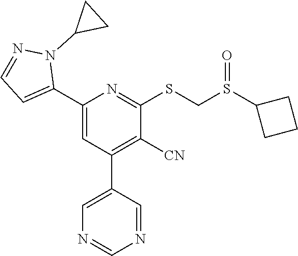 [SMILES: N#Cc1c(-c2cncnc2)cc(-c2ccnn2C2CC2)nc1SCS(=O)C1CCC1]

EXAMPLE 323H: 2-(((cyclobutylsulfinyl)methyl)thio)-6-(1-cyclopropyl-1H-pyrazol-5-yl)-4-(pyrimidin-5-yl)nicotinonitrile. The title compound was prepared by the general procedure starting from EXAMPLE 323G (300 mg, 713 umol), hydrogen peroxide (1.21 g, 10.7 mmol, 1.03 mL, 30% purity) to give the crude title compound (310 mg, 710 umol) as a yellow solid.

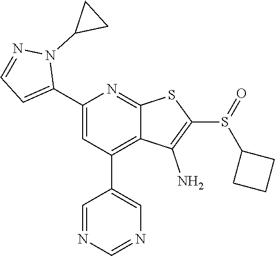 [SMILES: Nc1c(S(=O)C2CCC2)sc2nc(-c3ccnn3C3CC3)cc(-c3cncnc3)c12]

EXAMPLE 323: 2-(cyclobutylsulfinyl)-6-(1-cyclopropyl-1H-pyrazol-5-yl)-4-(pyrimidin-5-yl)thieno[2,3-b]pyridin-3-amine. The title compound was prepared by the general procedure starting from EXAMPLE 323H (300 mg, 687 umol) to give EXAMPLE 323 (125 mg, 279 umol, 40% yield, 97% purity) as a yellow solid. LCMS: (ES⁺) m/z (M)=437.1. HPLC: tR=1.925 min. ¹H NMR (400 MHz, CDCl3) δ 9.40 (s, 1H), 8.97 (s, 2H), 7.50 (d, J=2.0 Hz, 1H), 7.45 (s, 1H), 6.69 (d, J=2.0 Hz, 1H), 4.43 (s, 2H), 4.36-4.34 (m, 1H), 3.98-3.94 (m, 1H), 2.84-2.81 (m, 1H), 2.43-2.39 (m, 2H), 2.31-2.27 (m, 1H), 2.17-2.08 (m, 2H), 1.23-1.20 (m, 2H), 1.06-1.04 (m, 2H).

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

The following is claimed:

1. A compound of formula (IA): 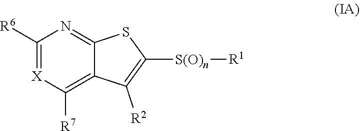
or a pharmaceutically acceptable salt, tautomer, or solvate thereof, wherein:
R¹ is alkyl, haloalkyl, cycloalkyl, alkylene-cycloalkyl, alkylene-alkoxy, heterocyclyl, or alkylene-heterocyclyl;
R² is —NH₂, CN, or —NHC(O)alkyl;
R⁶ is 5- to 6-membered heteroaryl optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-OH, —O-alkylene-N(R⁵)₂, —N(R⁵)₂, —N(R⁵)(alkylene-OH), —N(R⁵)(alkylene-O-alkyl), alkyl, -alkylene-OH, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl, wherein the cycloalkyl and the heterocyclyl is each optionally substituted with R¹⁰;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, -alkylene-O-alkyl, alkoxy, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
R¹⁰ is —OH, halogen, alkyl, or alkoxy;
X is N or CH;
in is 0, 1, or 2;
n is 0, 1, or 2; and
t is 0, 1, or 2;
wherein the compound is one not having a formula selected from the group consisting of:

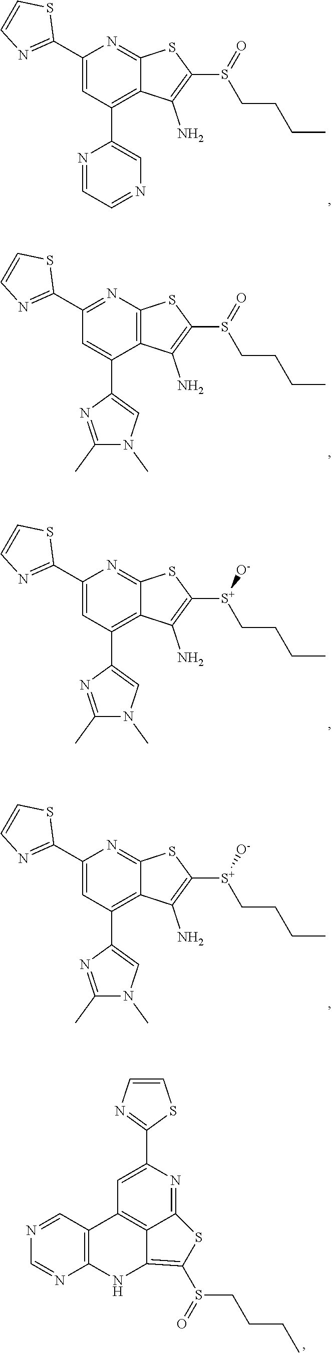 [SMILES: CCCCS(=O)c1sc2nc(-c3nccs3)cc(-c3cnccn3)c2c1N ; CCCCS(=O)c1sc2nc(-c3nccs3)cc(-c3cn(C)c(C)n3)c2c1N ; CCCC[S@+]([O-])c1sc2nc(-c3nccs3)cc(-c3cn(C)c(C)n3)c2c1N ; CCCC[S@@+]([O-])c1sc2nc(-c3nccs3)cc(-c3cn(C)c(C)n3)c2c1N ; CCCCS(=O)c1sc2nc(-c3nccs3)cc3c2c1Nc1ncncc1-3]

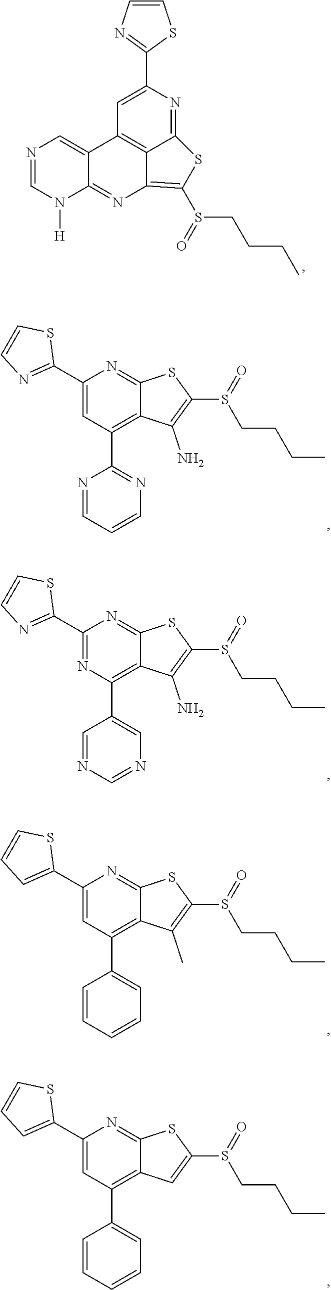 [SMILES: CCCCS(=O)c1sc2nc(-c3nccs3)cc3c4c(nc1c23)NC=NC=4 ; CCCCS(=O)c1sc2nc(-c3nccs3)cc(-c3ncccn3)c2c1N ; CCCCS(=O)c1sc2nc(-c3nccs3)nc(-c3cncnc3)c2c1N ; CCCCS(=O)c1sc2nc(-c3cccs3)cc(-c3ccccc3)c2c1C ; CCCCS(=O)c1cc2c(-c3ccccc3)cc(-c3cccs3)nc2s1]

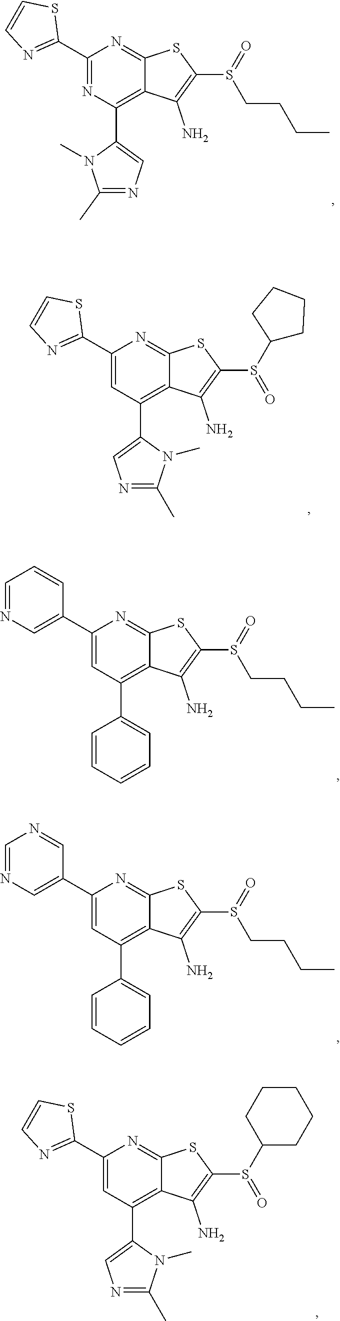 [SMILES: CCCCS(=O)c1sc2nc(-c3nccs3)nc(-c3cnc(C)n3C)c2c1N ; Cc1ncc(-c2cc(-c3nccs3)nc3sc(S(=O)C4CCCC4)c(N)c23)n1C ; CCCCS(=O)c1sc2nc(-c3cccnc3)cc(-c3ccccc3)c2c1N ; CCCCS(=O)c1sc2nc(-c3cncnc3)cc(-c3ccccc3)c2c1N ; Cc1ncc(-c2cc(-c3nccs3)nc3sc(S(=O)C4CCCCC4)c(N)c23)n1C]

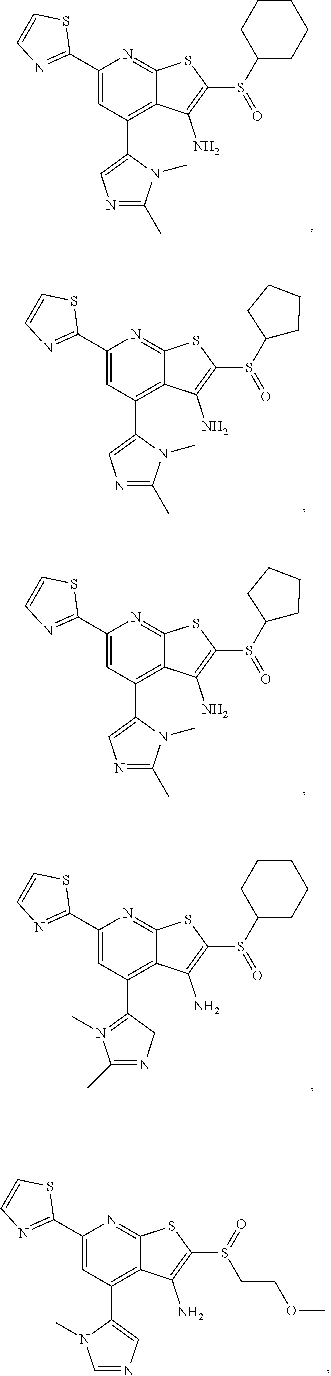

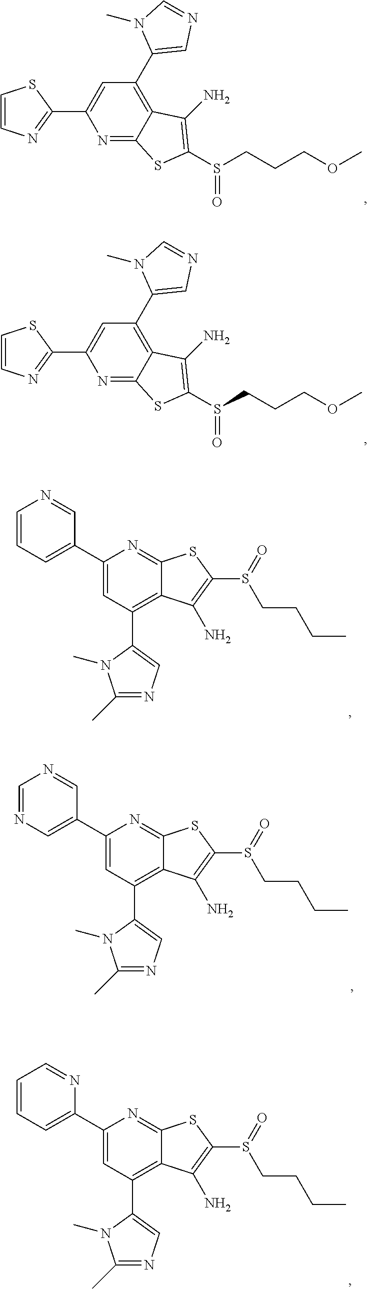 [SMILES: COCCCS(=O)c1sc2nc(-c3nccs3)cc(-c3cncn3C)c2c1N ; COCCC[S@@](=O)c1sc2nc(-c3nccs3)cc(-c3cncn3C)c2c1N ; CCCCS(=O)c1sc2nc(-c3cccnc3)cc(-c3cnc(C)n3C)c2c1N ; CCCCS(=O)c1sc2nc(-c3cncnc3)cc(-c3cnc(C)n3C)c2c1N ; CCCCS(=O)c1sc2nc(-c3ccccn3)cc(-c3cnc(C)n3C)c2c1N]

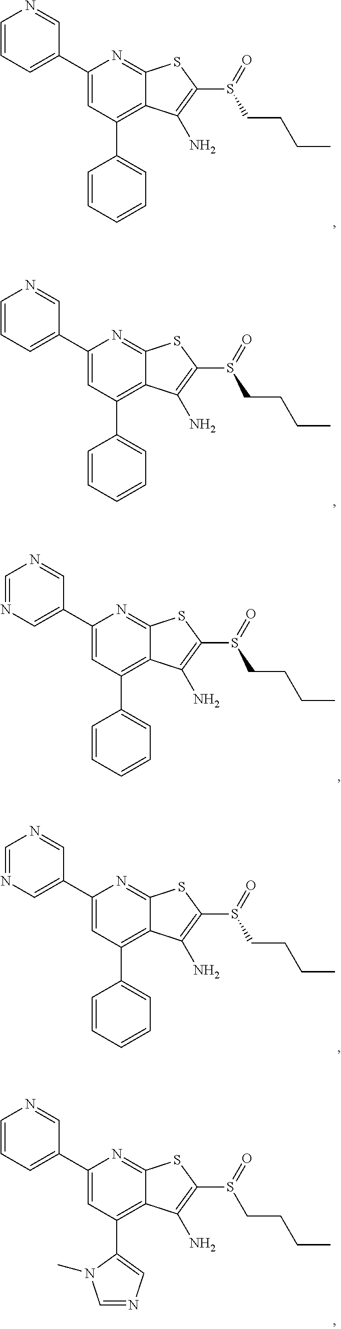 [SMILES: CCCC[S@@](=O)c1sc2nc(-c3cccnc3)cc(-c3ccccc3)c2c1N ; CCCC[S@](=O)c1sc2nc(-c3cccnc3)cc(-c3ccccc3)c2c1N ; CCCC[S@](=O)c1sc2nc(-c3cncnc3)cc(-c3ccccc3)c2c1N ; CCCC[S@@](=O)c1sc2nc(-c3cncnc3)cc(-c3ccccc3)c2c1N ; CCCCS(=O)c1sc2nc(-c3cccnc3)cc(-c3cncn3C)c2c1N]

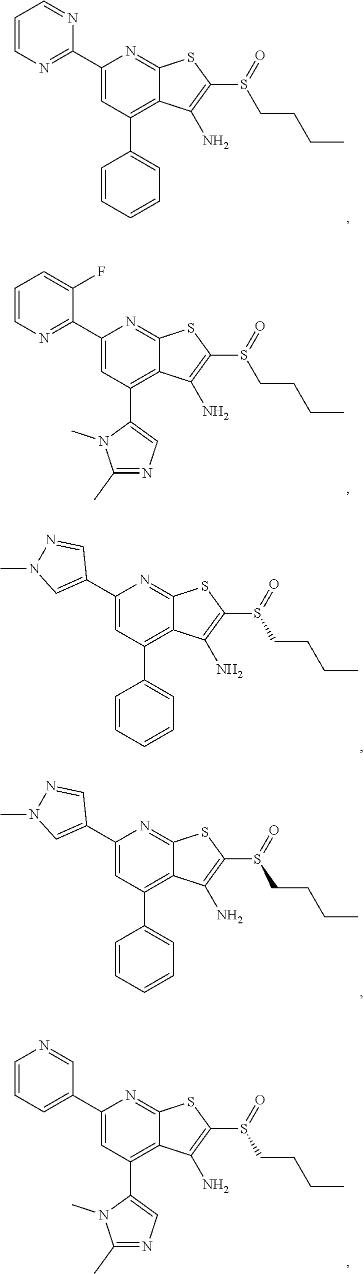

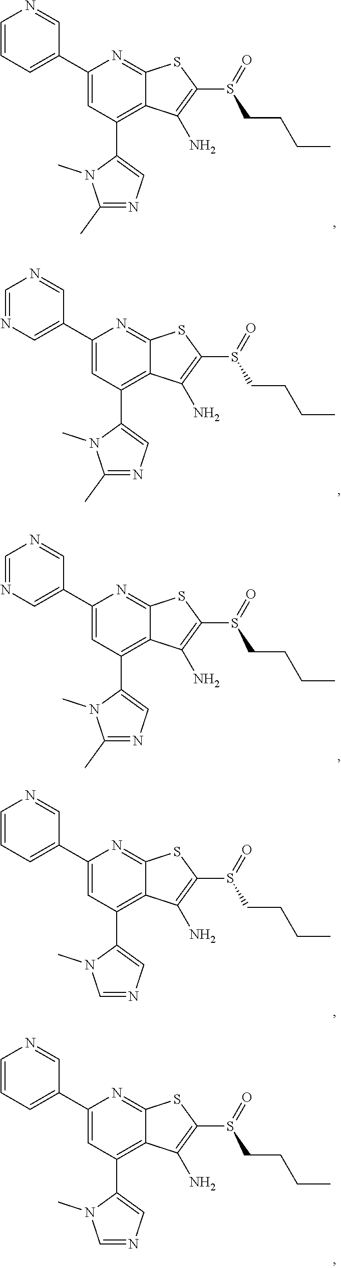 [SMILES: CCCC[S@](=O)c1sc2nc(-c3cccnc3)cc(-c3cnc(C)n3C)c2c1N ; CCCC[S@@](=O)c1sc2nc(-c3cncnc3)cc(-c3cnc(C)n3C)c2c1N ; CCCC[S@](=O)c1sc2nc(-c3cncnc3)cc(-c3cnc(C)n3C)c2c1N ; CCCC[S@@](=O)c1sc2nc(-c3cccnc3)cc(-c3cncn3C)c2c1N ; CCCC[S@](=O)c1sc2nc(-c3cccnc3)cc(-c3cncn3C)c2c1N]

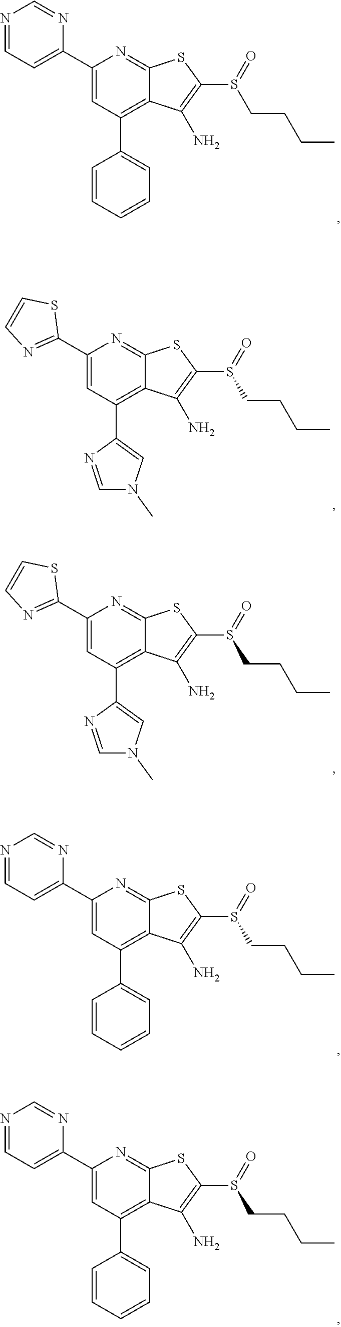 [SMILES: CCCCS(=O)c1sc2nc(-c3ccncn3)cc(-c3ccccc3)c2c1N ; CCCC[S@@](=O)c1sc2nc(-c3nccs3)cc(-c3cn(C)cn3)c2c1N ; CCCC[S@](=O)c1sc2nc(-c3nccs3)cc(-c3cn(C)cn3)c2c1N ; CCCC[S@@](=O)c1sc2nc(-c3ccncn3)cc(-c3ccccc3)c2c1N ; CCCC[S@](=O)c1sc2nc(-c3ccncn3)cc(-c3ccccc3)c2c1N]

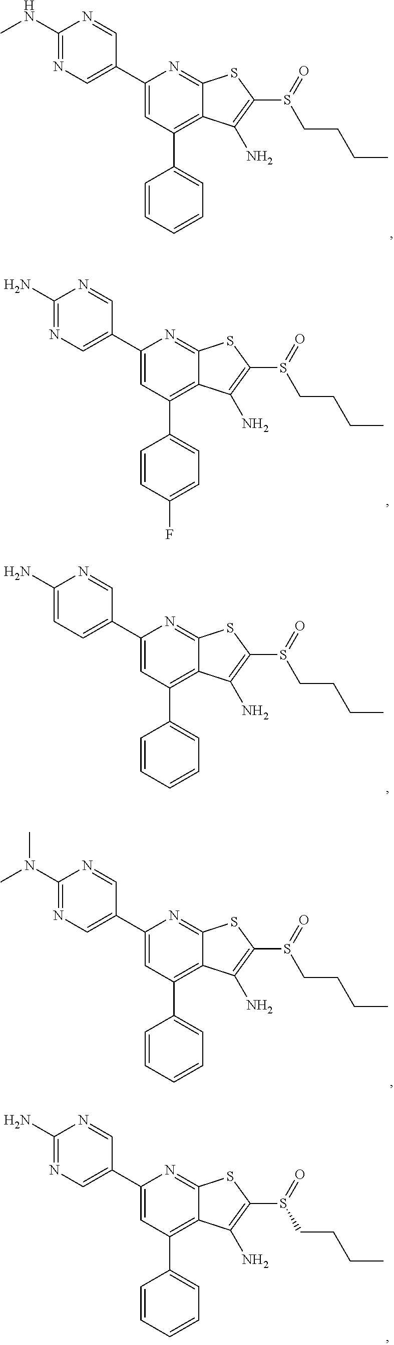

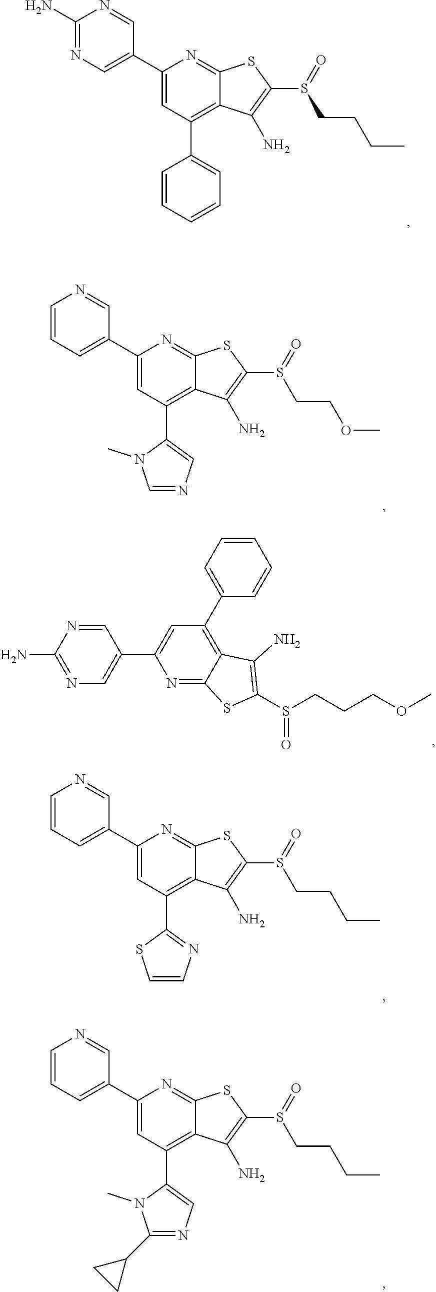 [SMILES: CCCC[S@](=O)c1sc2nc(-c3cnc(N)nc3)cc(-c3ccccc3)c2c1N ; COCCS(=O)c1sc2nc(-c3cccnc3)cc(-c3cncn3C)c2c1N ; COCCCS(=O)c1sc2nc(-c3cnc(N)nc3)cc(-c3ccccc3)c2c1N ; CCCCS(=O)c1sc2nc(-c3cccnc3)cc(-c3nccs3)c2c1N ; CCCCS(=O)c1sc2nc(-c3cccnc3)cc(-c3cnc(C4CC4)n3C)c2c1N]

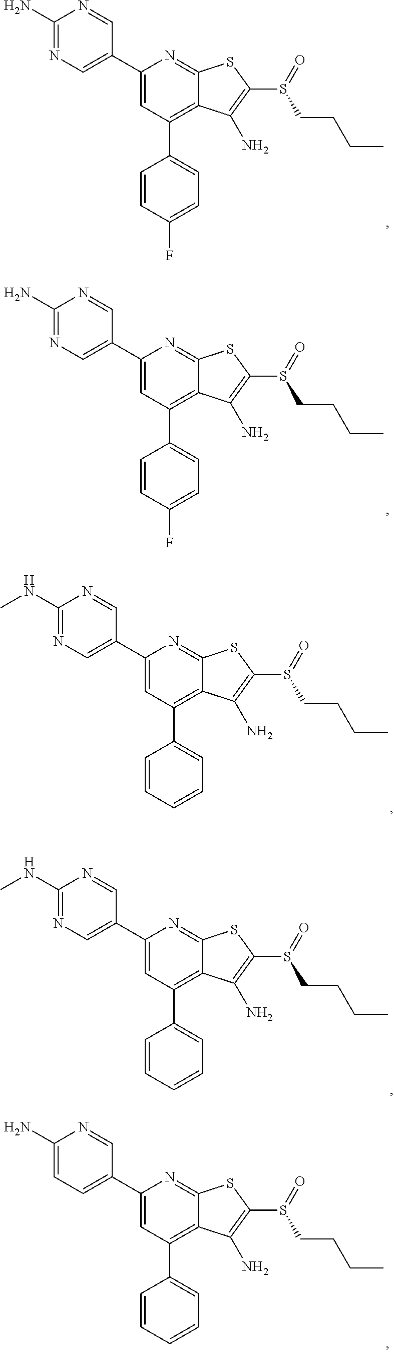

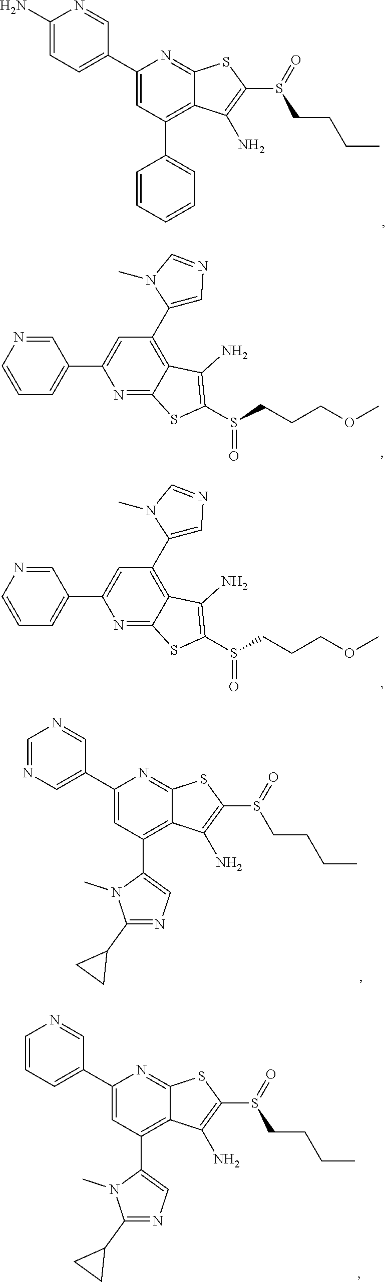

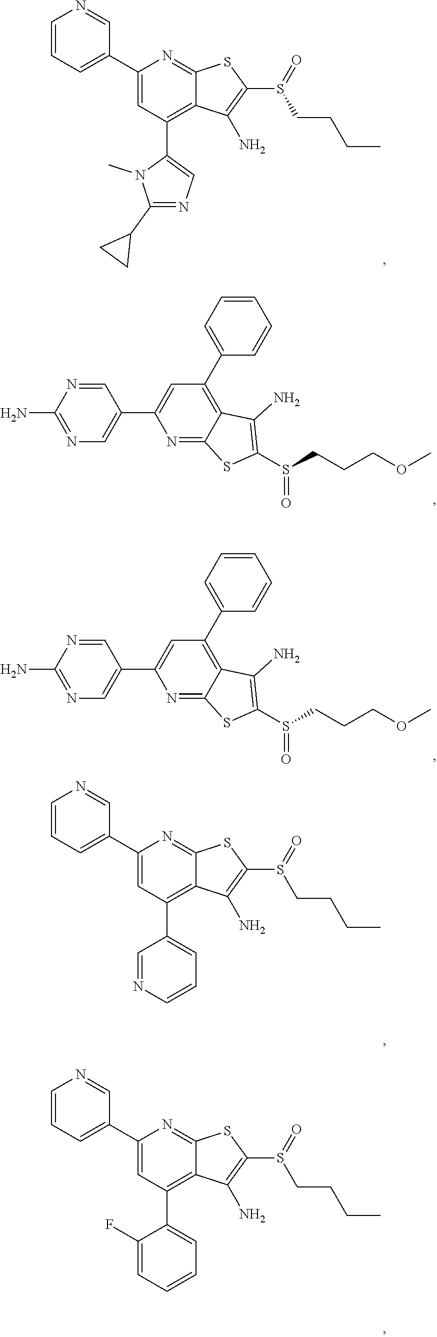 [SMILES: CCCC[S@@](=O)c1sc2nc(-c3cccnc3)cc(-c3cnc(C4CC4)n3C)c2c1N ; COCCC[S@@](=O)c1sc2nc(-c3cnc(N)nc3)cc(-c3ccccc3)c2c1N ; COCCC[S@](=O)c1sc2nc(-c3cnc(N)nc3)cc(-c3ccccc3)c2c1N ; CCCCS(=O)c1sc2nc(-c3cccnc3)cc(-c3cccnc3)c2c1N ; CCCCS(=O)c1sc2nc(-c3cccnc3)cc(-c3ccccc3F)c2c1N]

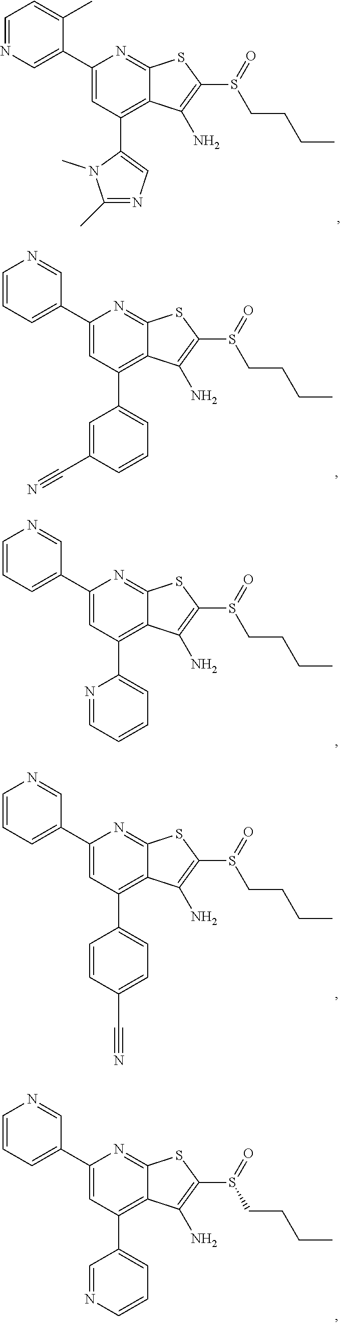

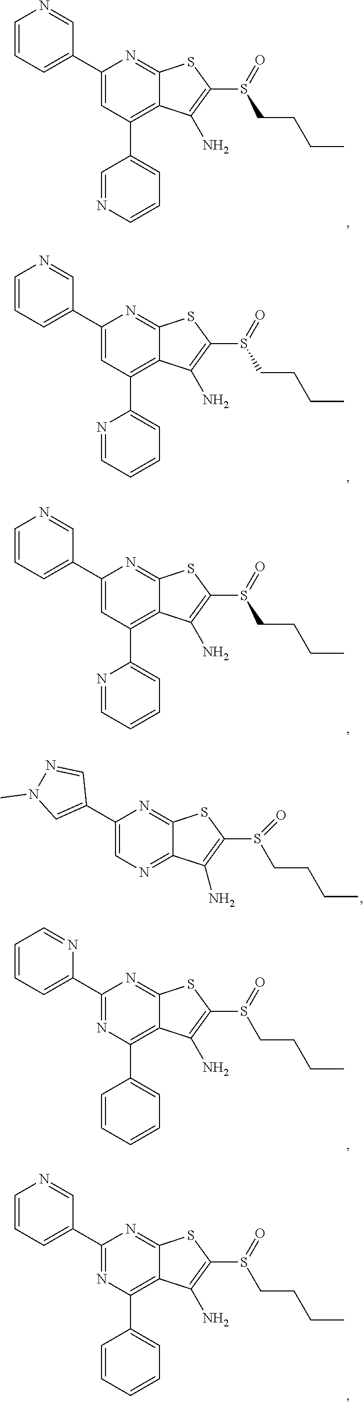

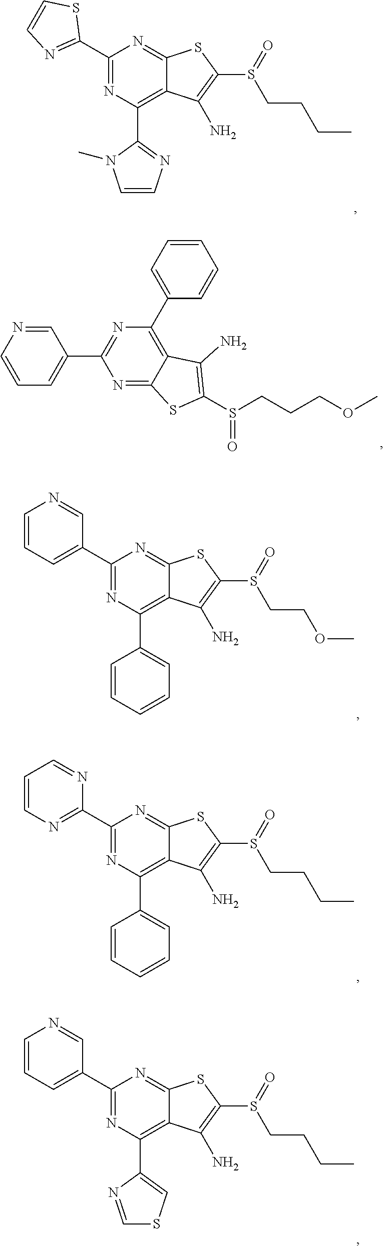 [SMILES: CCCCS(=O)c1sc2nc(-c3nccs3)nc(-c3nccn3C)c2c1N ; COCCCS(=O)c1sc2nc(-c3cccnc3)nc(-c3ccccc3)c2c1N ; COCCS(=O)c1sc2nc(-c3cccnc3)nc(-c3ccccc3)c2c1N ; CCCCS(=O)c1sc2nc(-c3ncccn3)nc(-c3ccccc3)c2c1N ; CCCCS(=O)c1sc2nc(-c3cccnc3)nc(-c3cscn3)c2c1N]

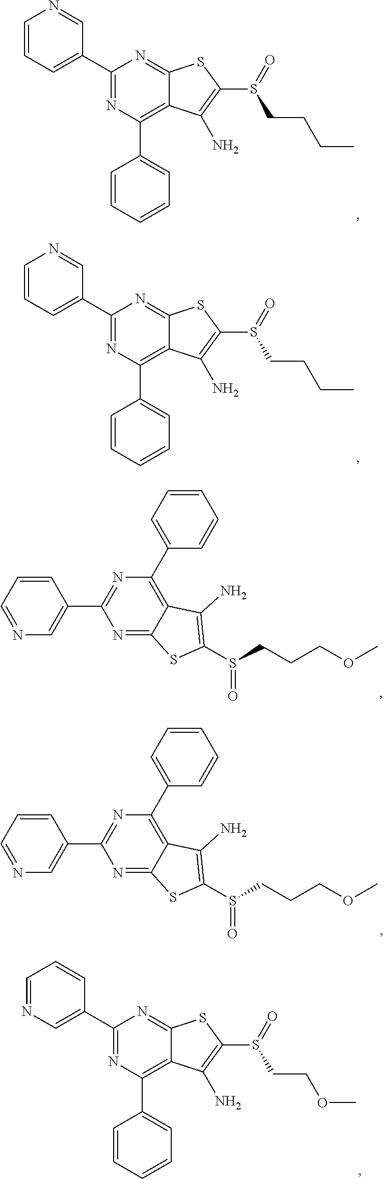

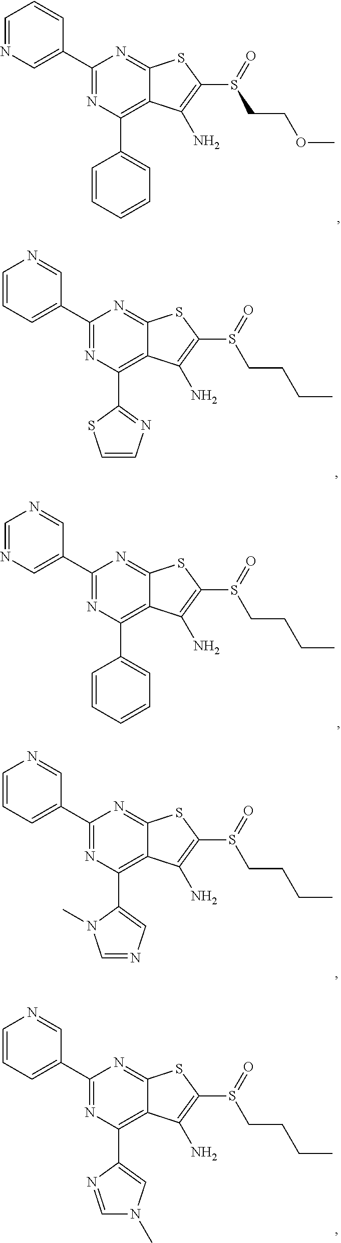 [SMILES: COCC[S@](=O)c1sc2nc(-c3cccnc3)nc(-c3ccccc3)c2c1N ; CCCCS(=O)c1sc2nc(-c3cccnc3)nc(-c3nccs3)c2c1N ; CCCCS(=O)c1sc2nc(-c3cncnc3)nc(-c3ccccc3)c2c1N ; CCCCS(=O)c1sc2nc(-c3cccnc3)nc(-c3cncn3C)c2c1N ; CCCCS(=O)c1sc2nc(-c3cccnc3)nc(-c3cn(C)cn3)c2c1N]

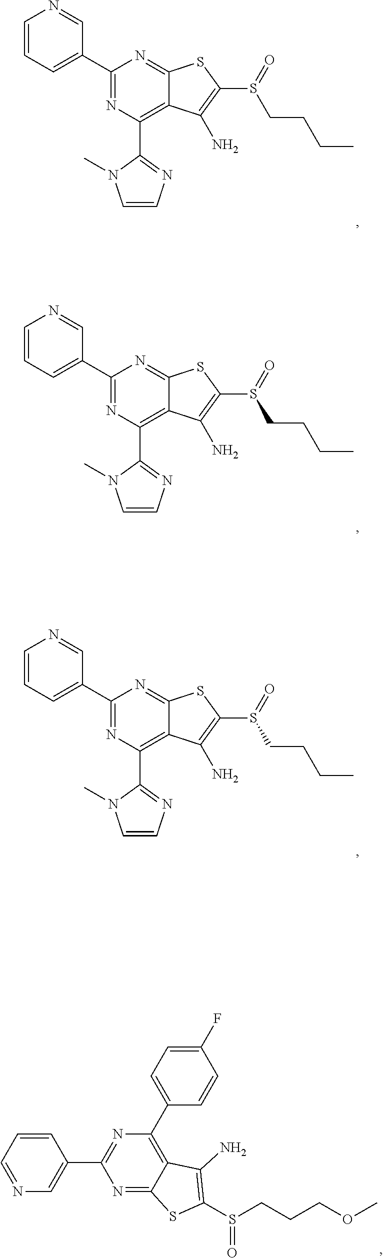 [SMILES: CCCCS(=O)c1sc2nc(-c3cccnc3)nc(-c3nccn3C)c2c1N ; CCCC[S@](=O)c1sc2nc(-c3cccnc3)nc(-c3nccn3C)c2c1N ; CCCC[S@@](=O)c1sc2nc(-c3cccnc3)nc(-c3nccn3C)c2c1N ; COCCCS(=O)c1sc2nc(-c3cccnc3)nc(-c3ccc(F)cc3)c2c1N]

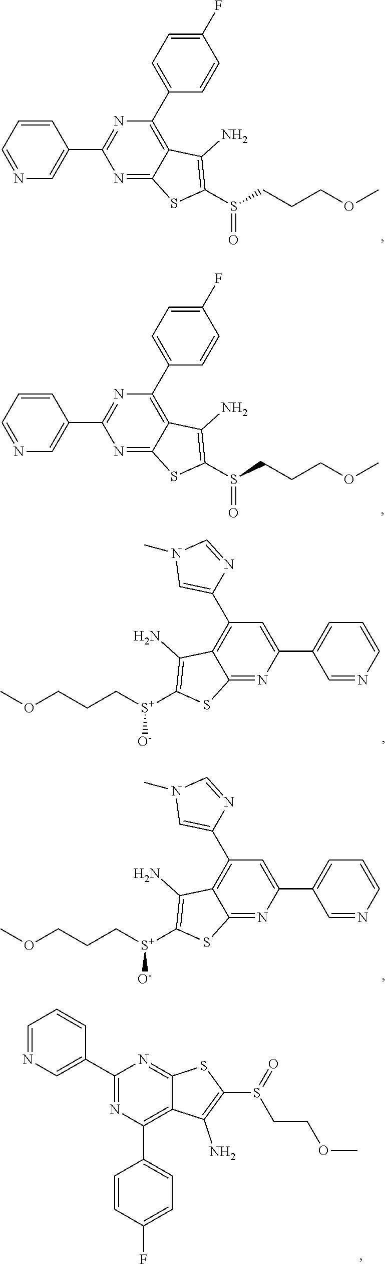

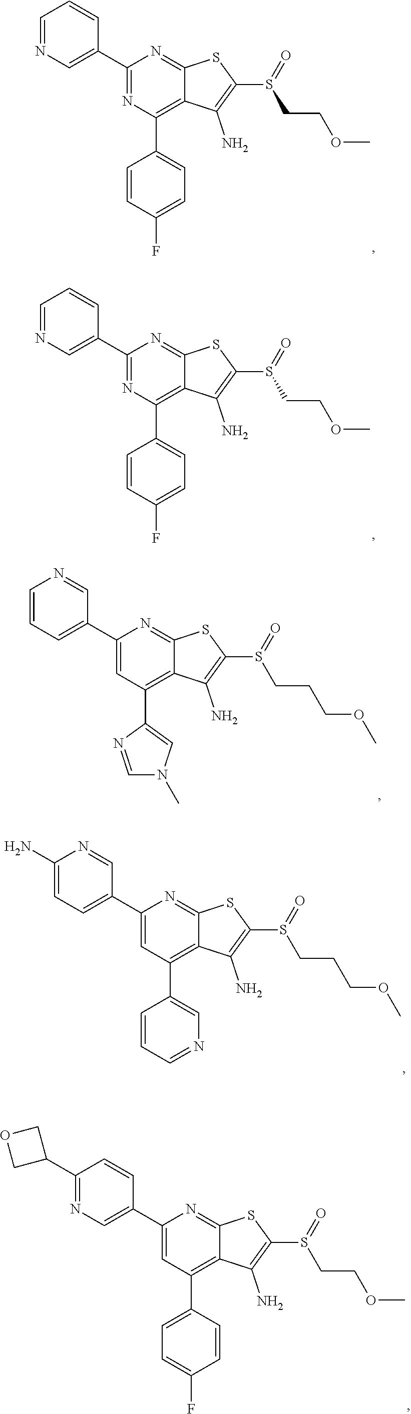 [SMILES: COCC[S@](=O)c1sc2nc(-c3cccnc3)nc(-c3ccc(F)cc3)c2c1N ; COCC[S@@](=O)c1sc2nc(-c3cccnc3)nc(-c3ccc(F)cc3)c2c1N ; COCCCS(=O)c1sc2nc(-c3cccnc3)cc(-c3cn(C)cn3)c2c1N ; COCCCS(=O)c1sc2nc(-c3ccc(N)nc3)cc(-c3cccnc3)c2c1N ; COCCS(=O)c1sc2nc(-c3ccc(C4COC4)nc3)cc(-c3ccc(F)cc3)c2c1N]

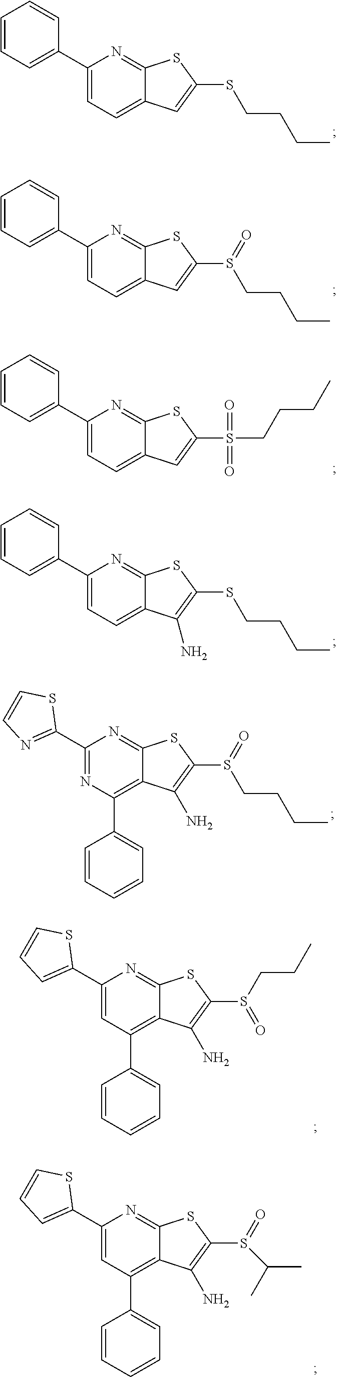

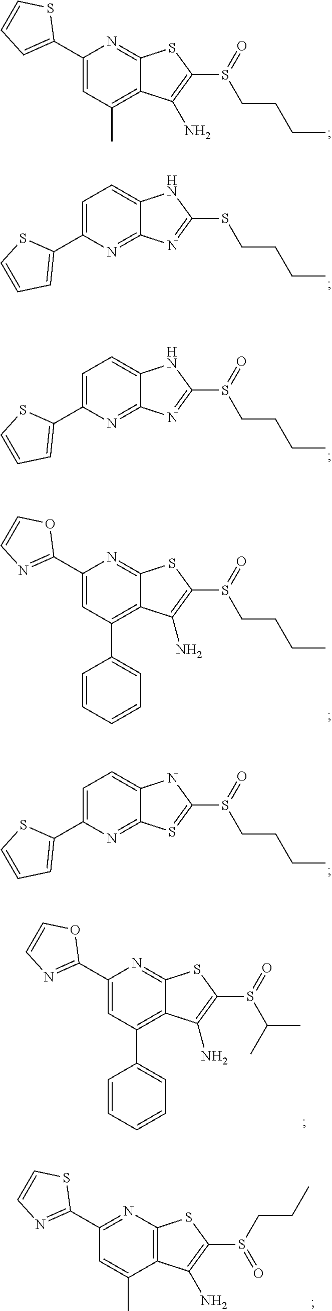

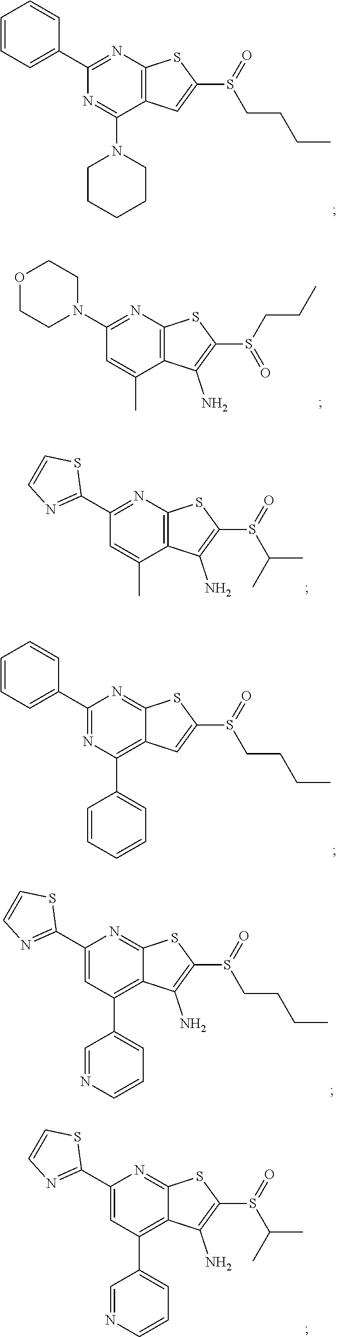 [SMILES: CCCCS(=O)c1cc2c(N3CCCCC3)nc(-c3ccccc3)nc2s1 ; CCCS(=O)c1sc2nc(N3CCOCC3)cc(C)c2c1N ; Cc1cc(-c2nccs2)nc2sc(S(=O)C(C)C)c(N)c12 ; CCCCS(=O)c1cc2c(-c3ccccc3)nc(-c3ccccc3)nc2s1 ; CCCCS(=O)c1sc2nc(-c3nccs3)cc(-c3cccnc3)c2c1N ; CC(C)S(=O)c1sc2nc(-c3nccs3)cc(-c3cccnc3)c2c1N]

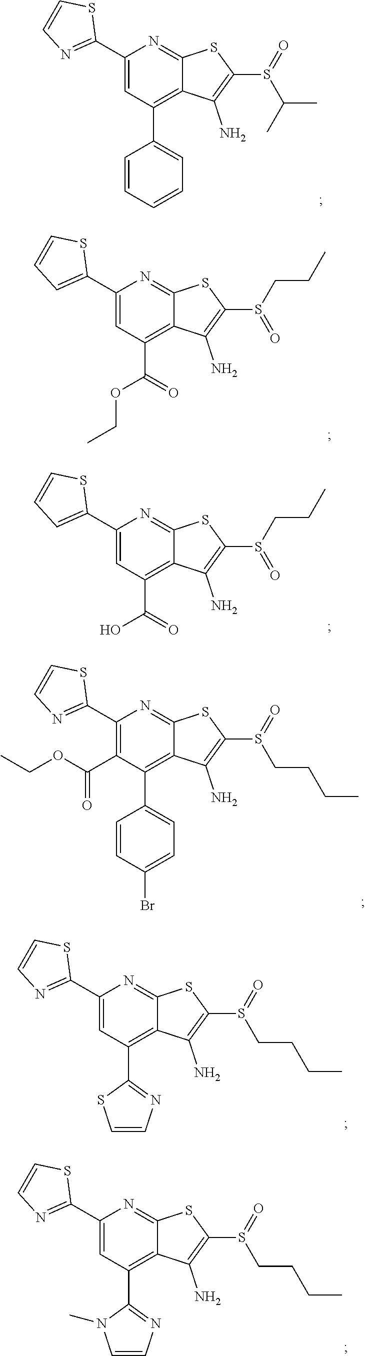

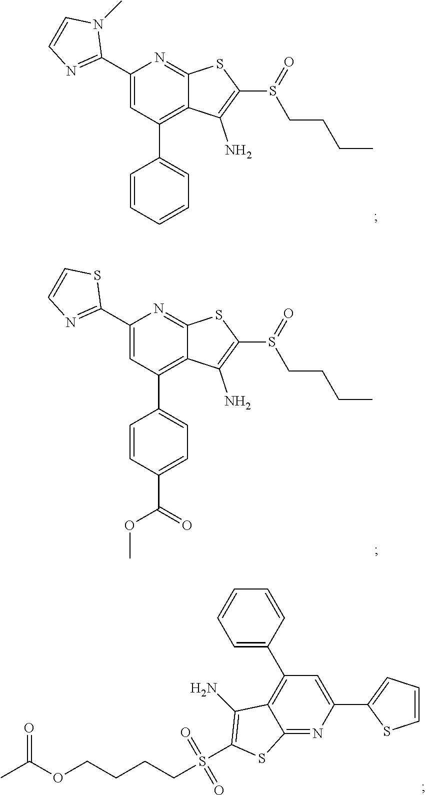

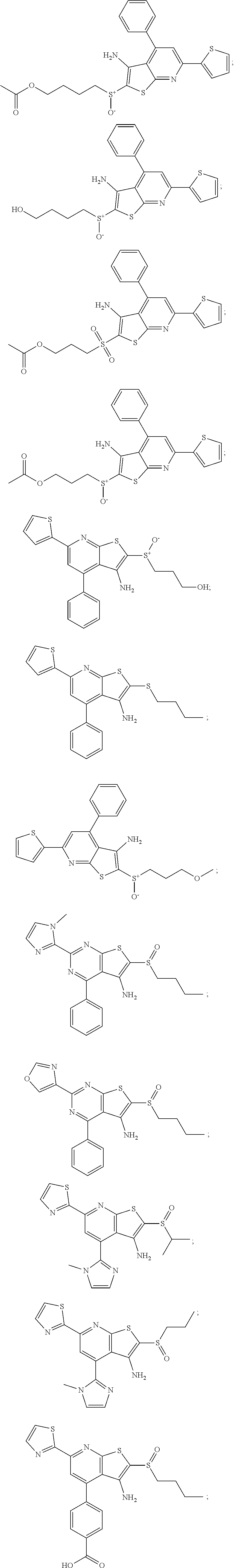

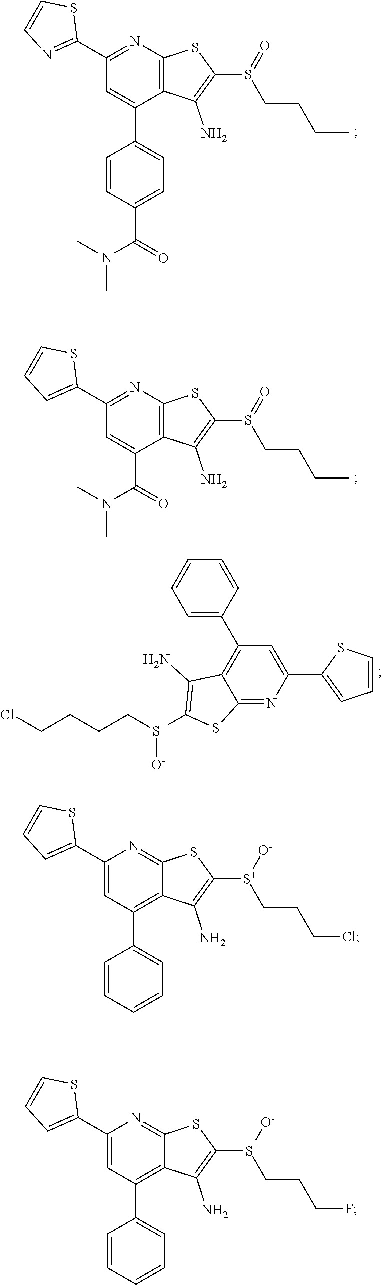 [SMILES: CCCCS(=O)c1sc2nc(-c3nccs3)cc(-c3ccc(C(=O)N(C)C)cc3)c2c1N ; CCCCS(=O)c1sc2nc(-c3cccs3)cc(C(=O)N(C)C)c2c1N ; Nc1c([S+]([O-])CCCCCl)sc2nc(-c3cccs3)cc(-c3ccccc3)c12 ; Nc1c([S+]([O-])CCCCl)sc2nc(-c3cccs3)cc(-c3ccccc3)c12 ; Nc1c([S+]([O-])CCCF)sc2nc(-c3cccs3)cc(-c3ccccc3)c12]

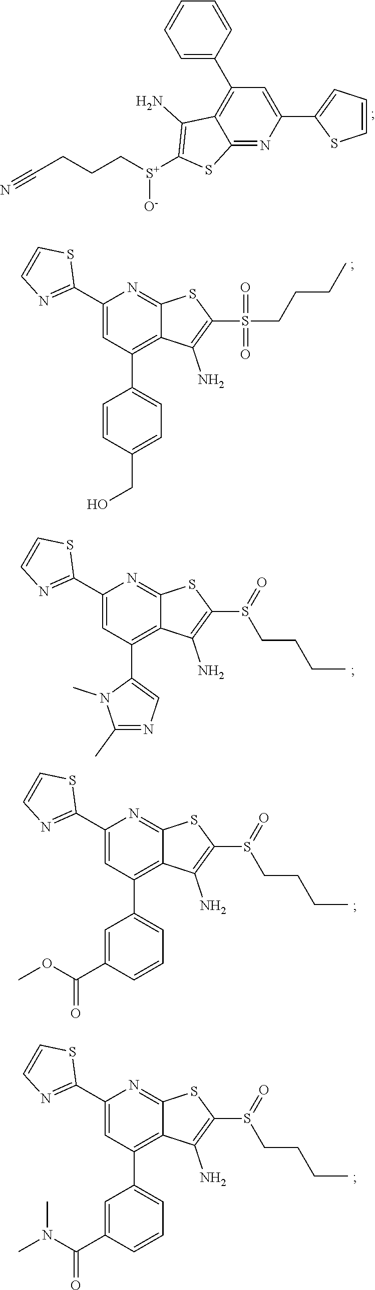

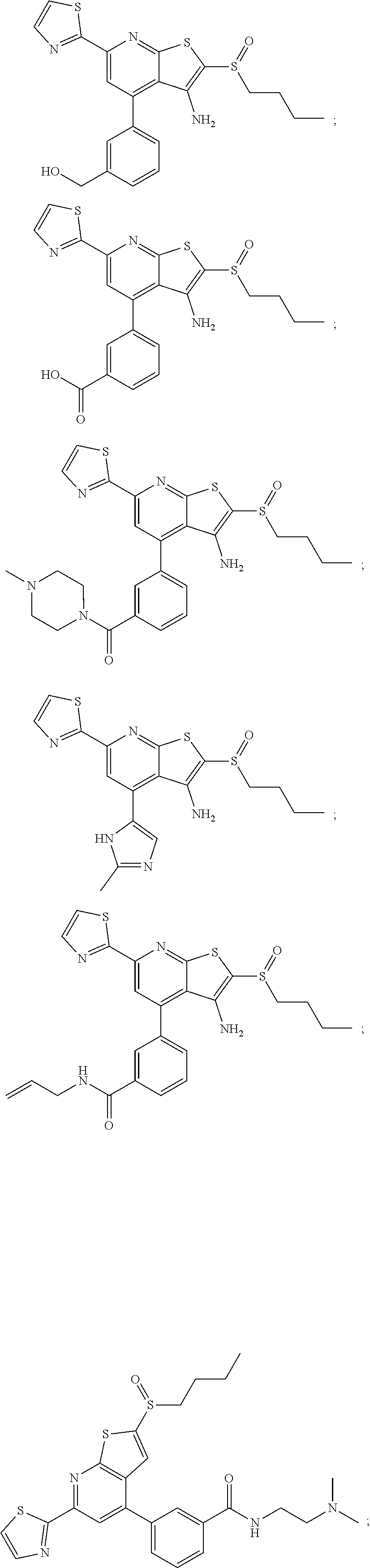

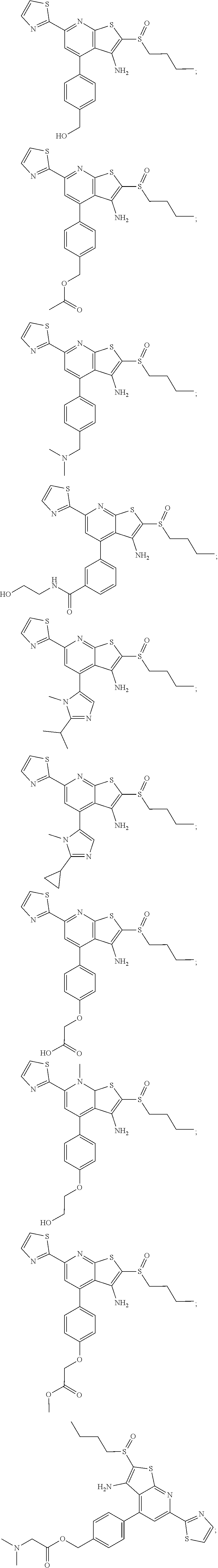

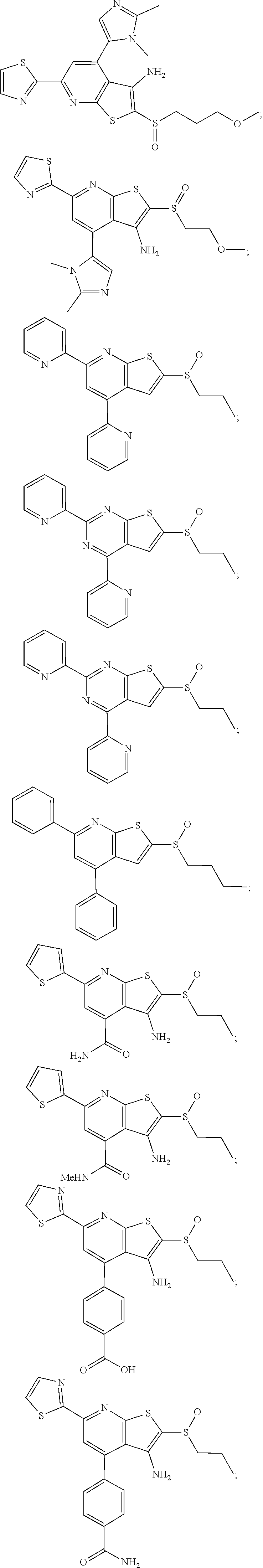

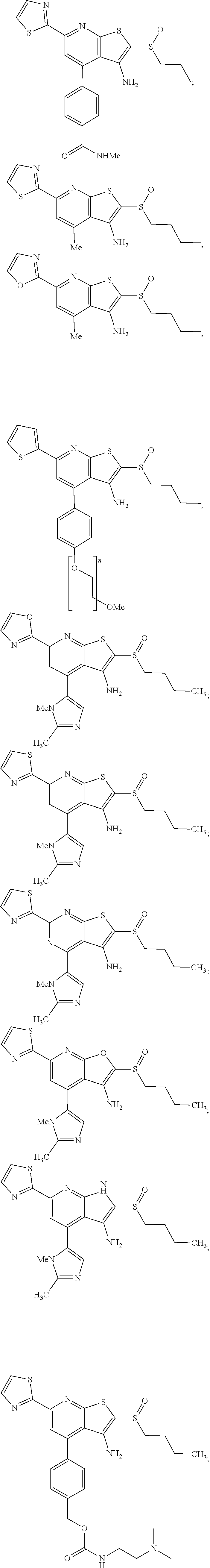

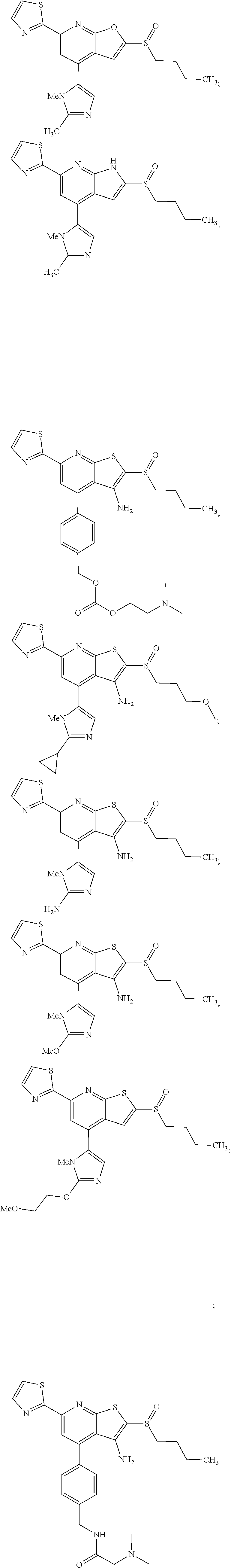

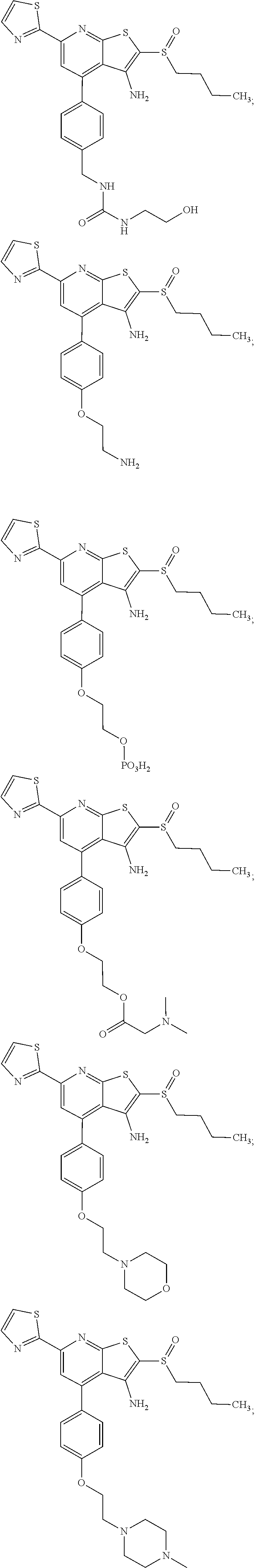

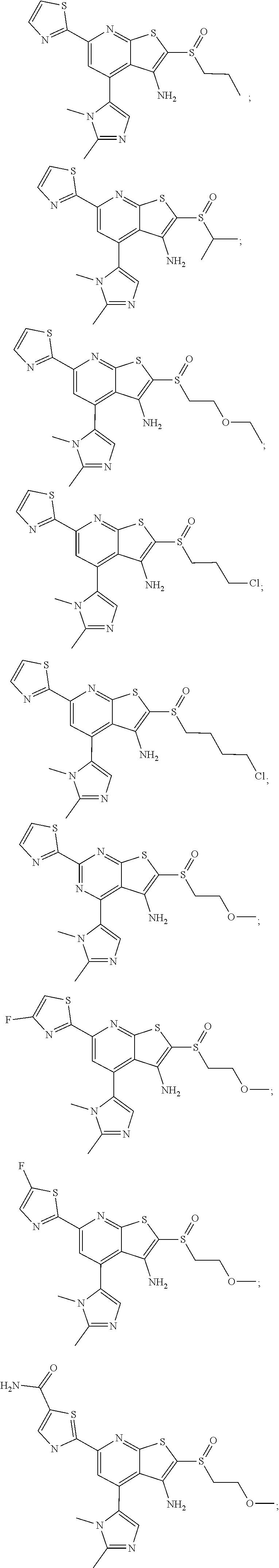

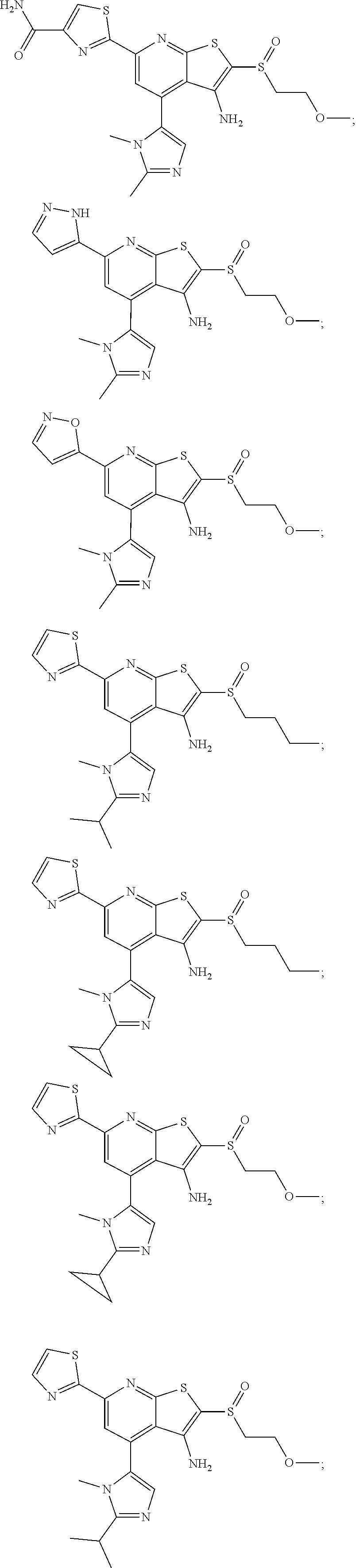

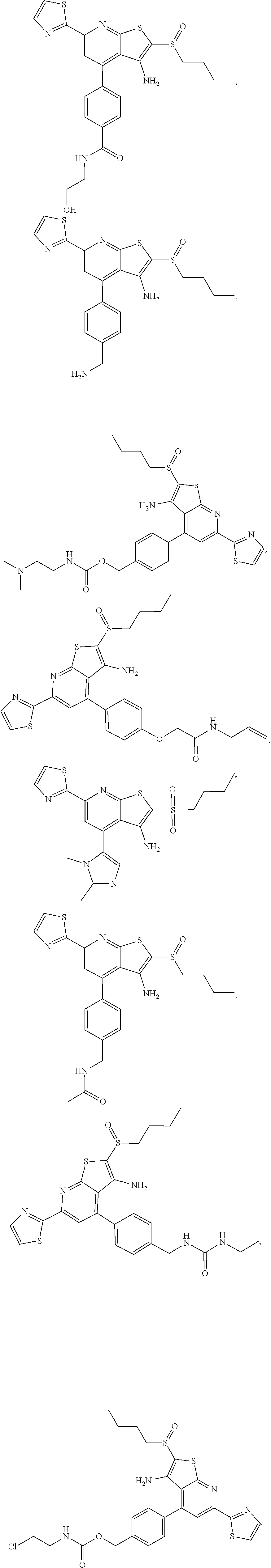

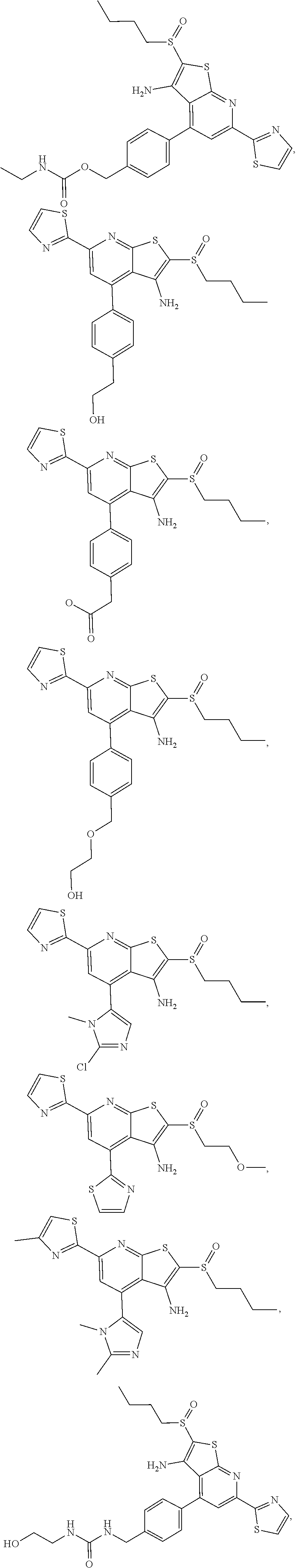

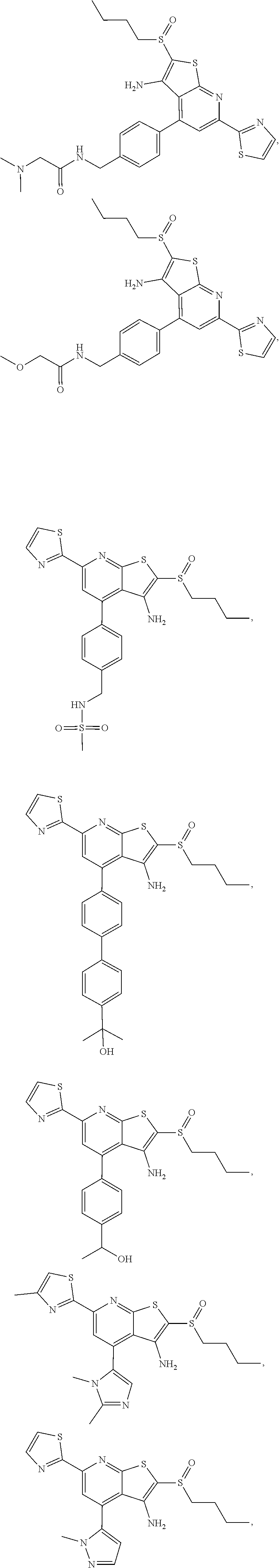

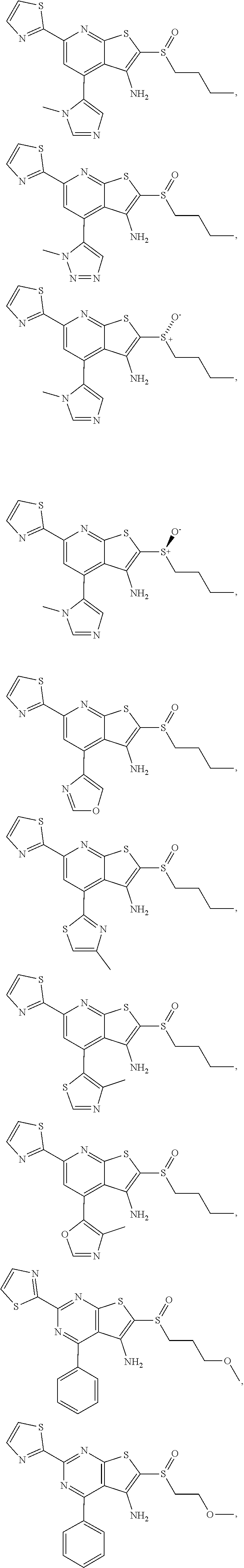

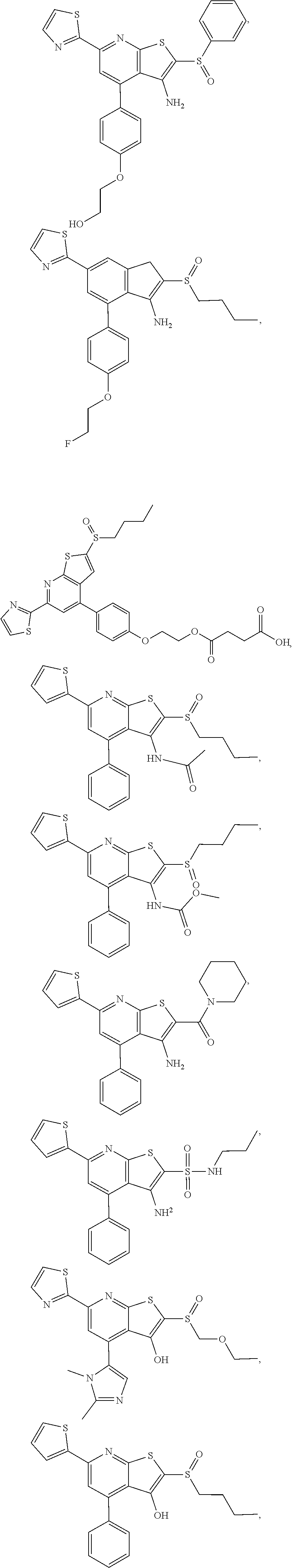

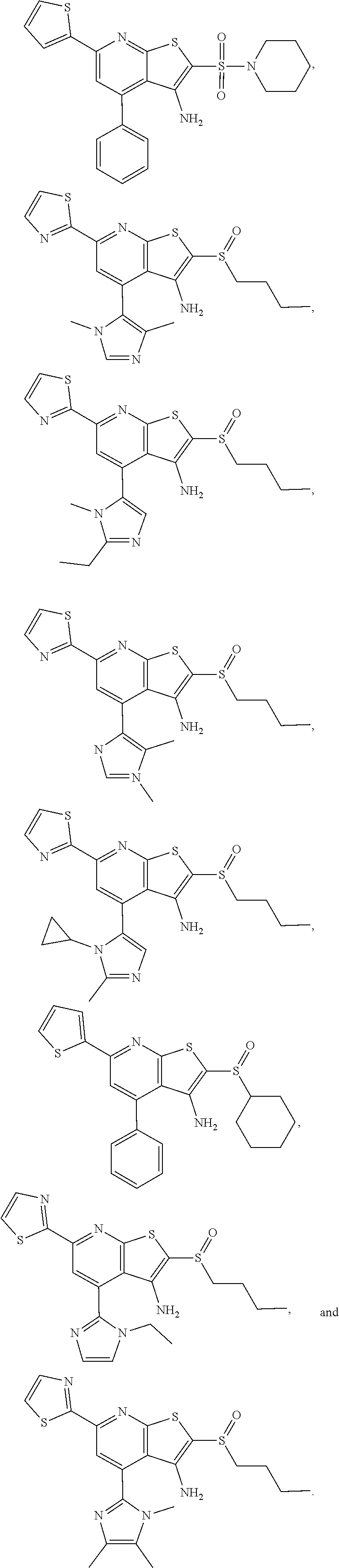

2. The compound, salt, or solvate of claim 1, wherein R¹ is C₁-C₆ alkyl, C₁-C₆ haloalkyl, 3- to 6-membered cycloalkyl, —(C₁-C₆ alkylene)-(3- to 6-membered cycloalkyl), —(C₁-C₆ alkylene)—(C₁-C₆ alkoxy), 3- to 6-membered heterocyclyl, or —(C₁-C₆ alkylene)-(3- to 6-membered heterocyclyl).

3. The compound, salt, or solvate of claim 1, wherein R² is —NH₂.

4. The compound, salt, or solvate of claim 1, wherein R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

5. The compound, salt, or solvate of claim 1, wherein R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

6. The compound, salt, or solvate of claim 1, wherein R⁴ is halogen, —CN, —N(R⁵)₂, —OH, —O—(C₁-C₆ alkylene)—OH, —S(O)ₘ(C₁-C₆ alkyl), —C(O)(C₁-C₆ alkyl), —C(O)-(3- to 6-membered cycloalkyl), C₁-C₆ alkyl, C₁-C₆ haloalkyl, 3- to 6-membered cycloalkyl, or 3- to 6-membered heterocyclyl.

7. The compound, salt, or solvate of claim 1, wherein n is 1.

8. The compound, salt, or solvate of claim 1, wherein the compound has the structure of formula (II): 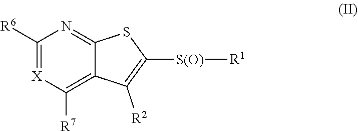
wherein:
R¹ is 3- to 6-membered cycloalkyl, —(C₁-C₆ alkylene)-(3- to 6-membered cycloalkyl), 3- to 6-membered heterocyclyl, or —(C₁-C₆ alkylene)-(3- to 6-membered heterocyclyl);
R² is —NH₂, CN, or —NHC(O)alkyl;
R⁶ is 5- to 6-membered heteroaryl optionally substituted with one or more R³;
R⁷ is alkyl, haloalkyl, cycloalkyl, aryl, heterocyclyl, heteroaryl, —C(O)-alkyl, —C(O)O-alkyl, or —C(O)NR⁵-alkyl, each of which is optionally substituted with one or more R⁴;
R³ is oxo, —OH, —O-alkylene-N(R⁵)₂, —N(R⁵)₂, —N(R⁵)(alkylene-OH), alkyl, haloalkyl, cycloalkyl, heterocyclyl, —C(O)N(R⁵)₂, —C(O)N(R⁵)(alkylene-OH), —C(O)-alkyl, —C(O)O-alkyl, or —S(O)ₘ-alkyl;
R⁴ is oxo, halogen, —CN, —N(R⁵)₂, —OH, —O-alkylene-OH, —S(O)ₘ-alkyl, —C(O)-alkyl, —C(O)-cycloalkyl, alkyl, haloalkyl, cycloalkyl, heterocyclyl, or -alkylene-aryl optionally substituted with R⁸;
each R⁵ is independently, H, alkyl, -alkylene-OH optionally substituted with —OH, -alkylene-NH₂, -alkylene-N(R⁹)₂, -alkylene-O-alkylene-OH, -alkylene-O-alkylene-NH₂, —C(O)-alkyl, —C(O)O-alkyl, -alkylene-COOH, or —S(O)ₘ-alkyl;
or alternatively, two R⁵ together with the N atom to which they are attached can form a 4- to 7-membered heterocycle, optionally containing an additional heteroatom selected from O, S, or N, and wherein the heterocycle is optionally substituted with R⁸;
R⁸ is halogen, alkyl, or alkoxy;
R⁹ is H or alkyl, or two R⁹ together with the N atom to which they are attached can form a 4- to 7-membered heterocycle, optionally containing an additional heteroatom selected from O, S(O)ₜ, or N;
X is N or CH;
m is 0, 1, or 2; and
t is 0, 1, or 2.

9. The compound, salt, or solvate of claim 8, wherein R¹ is 3- to 5-membered cycloalkyl or —(C₁-C₆ alkylene)-(3- to 5-membered cycloalkyl).

10. The compound, salt, or solvate of claim 8, wherein R¹ is cyclobutyl.

11. The compound, salt, or solvate of claim 8, wherein R¹ is a bicyclic 4- to 6-membered cycloalkyl.

12. The compound, salt, or solvate of claim 8, wherein R⁷ is C₁-C₆ alkyl, C₁-C₆ haloalkyl, 3- to 6-membered cycloalkyl, 6- to 10-membered aryl, 3- to 6-membered heterocyclyl, 5- to 10-membered heteroaryl, —C(O)(C₁-C₆ alkyl), —C(O)O(C₁-C₆ alkyl), or —C(O)NR⁵(C₁-C₆ alkyl), each of which is optionally substituted with one or more R⁴.

13. The compound, salt, or solvate of claim 8, wherein R⁷ is C₁-C₄ alkyl, C₁-C₆ haloalkyl, 3- to 6-membered cycloalkyl, phenyl, 3- to 6-membered heterocyclyl, or 5- to 10-membered heteroaryl, each of which is optionally substituted with one or more R⁴.

14. The compound, salt, or solvate of claim 8, wherein R³ is —O—(C₁-C₆ alkylene)-N(R⁵)₂, —N(R⁵)₂, —N(R⁵)(C₁-C₆ alkylene-OH), —C(O)N(R⁵)₂, —C(O)N(R⁵)(C₁-C₆ alkylene-OH), —C(O)(C₁-C₆ alkyl), —C(O)O(C₁-C₆ alkyl), or —S(O)ₘ(C₁-C₆ alkyl).

15. The compound, salt, or solvate of claim 1, selected from the group consisting of:

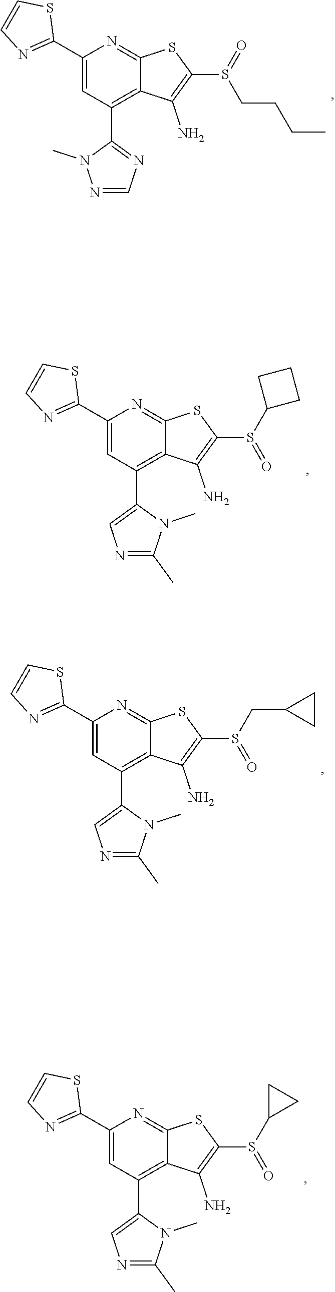

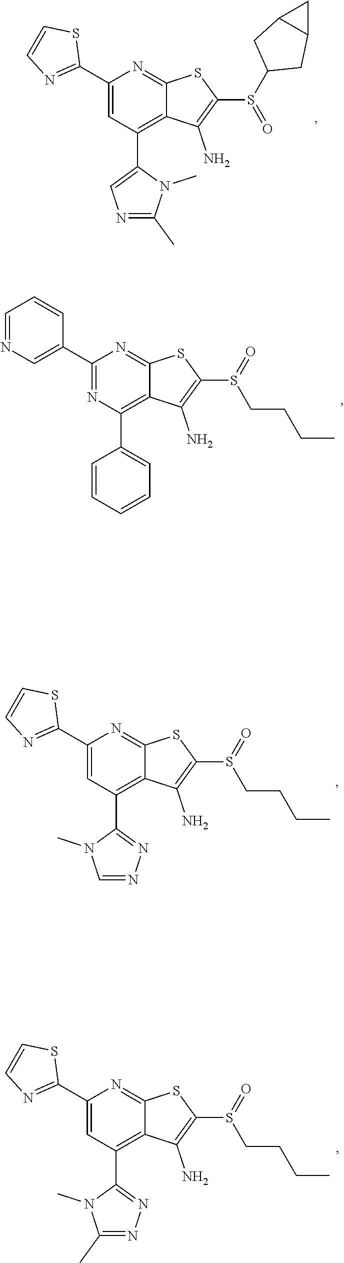

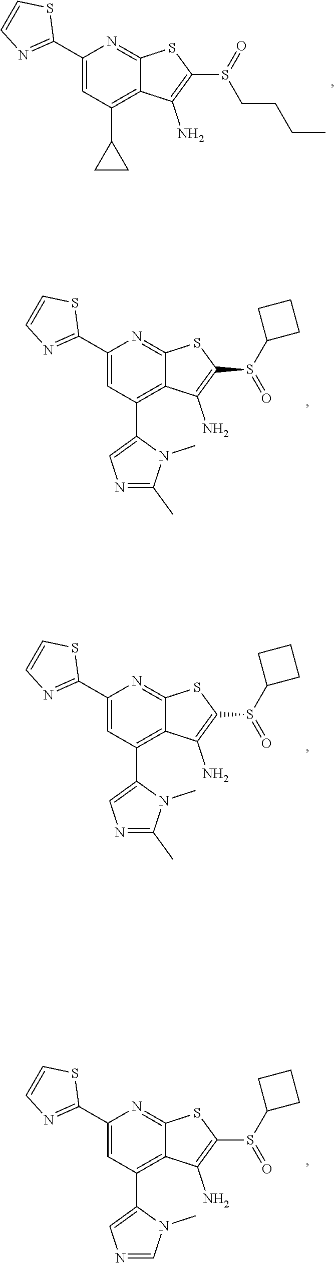

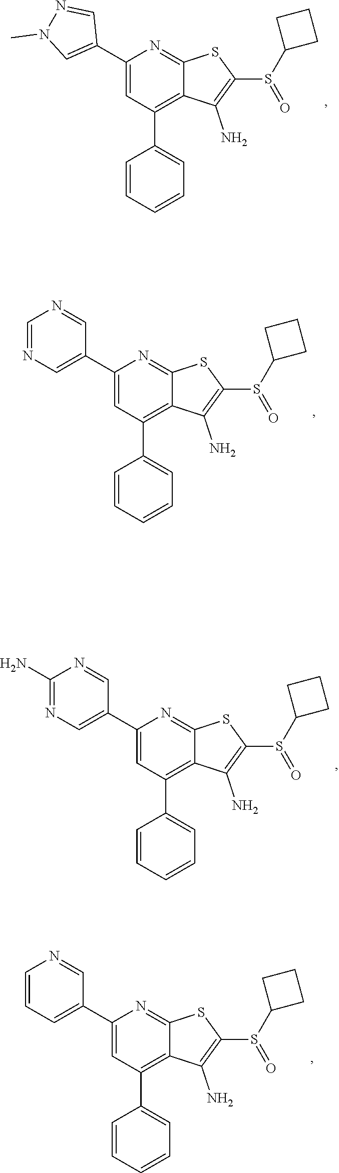

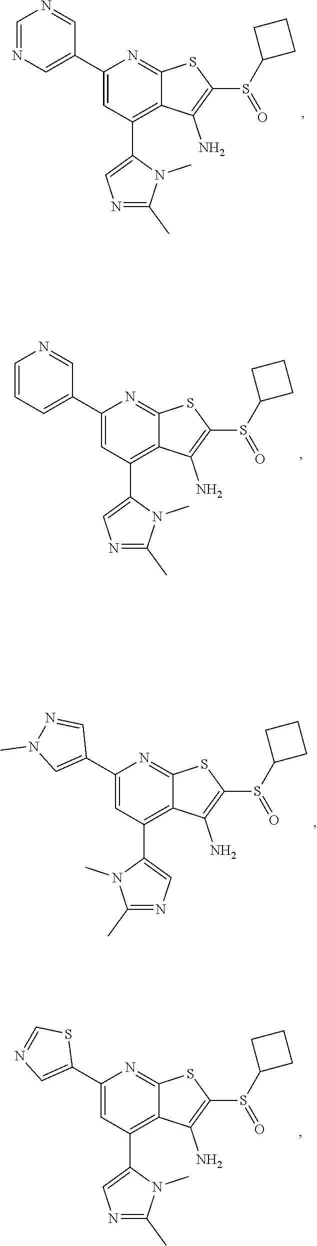

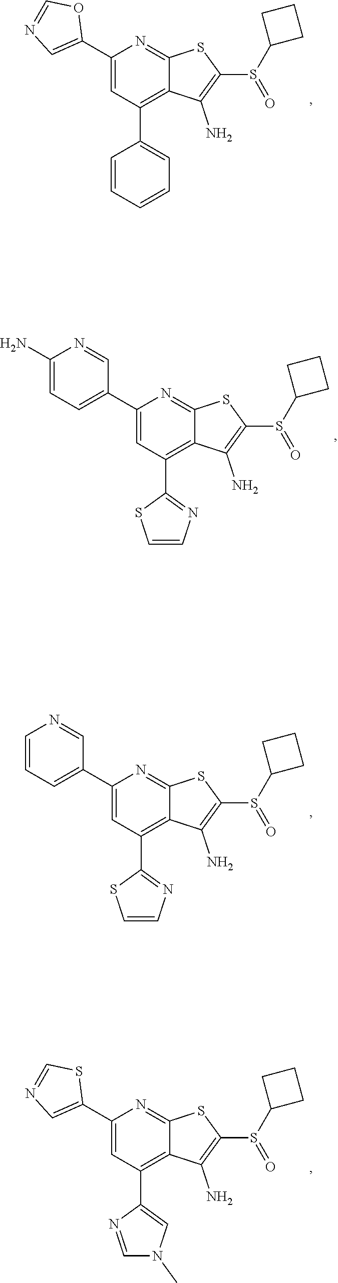

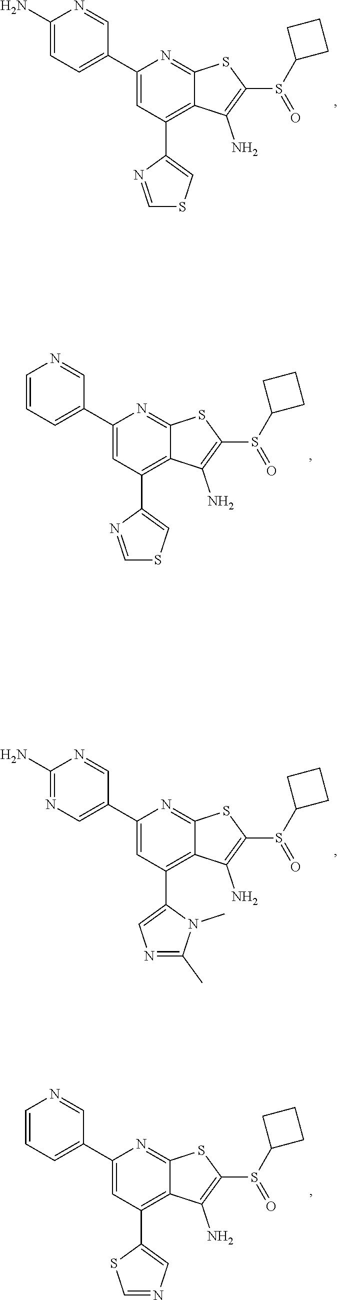

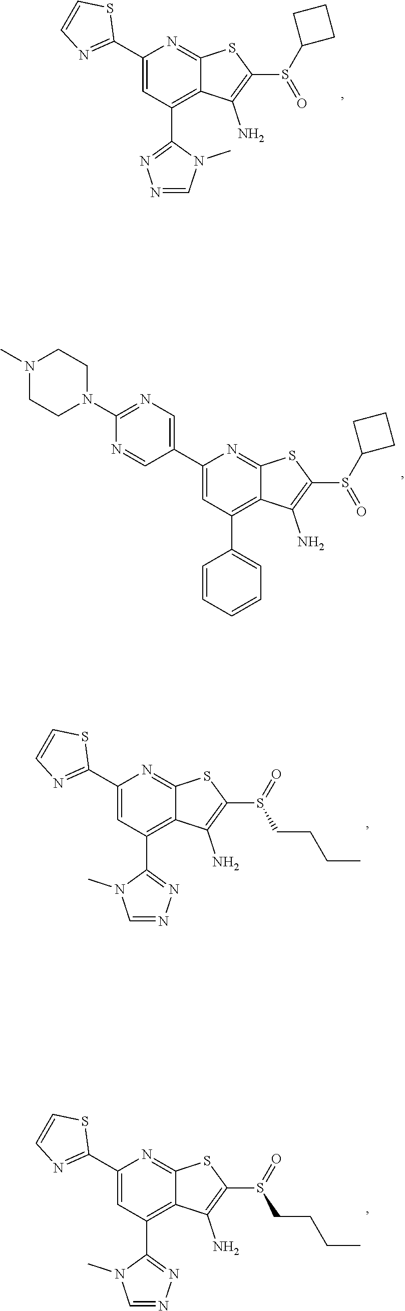

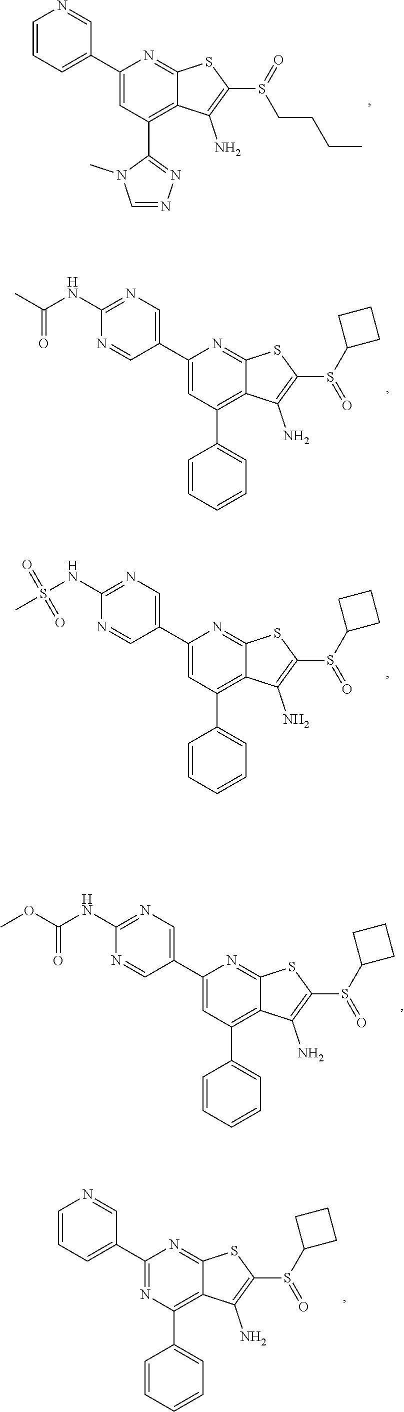

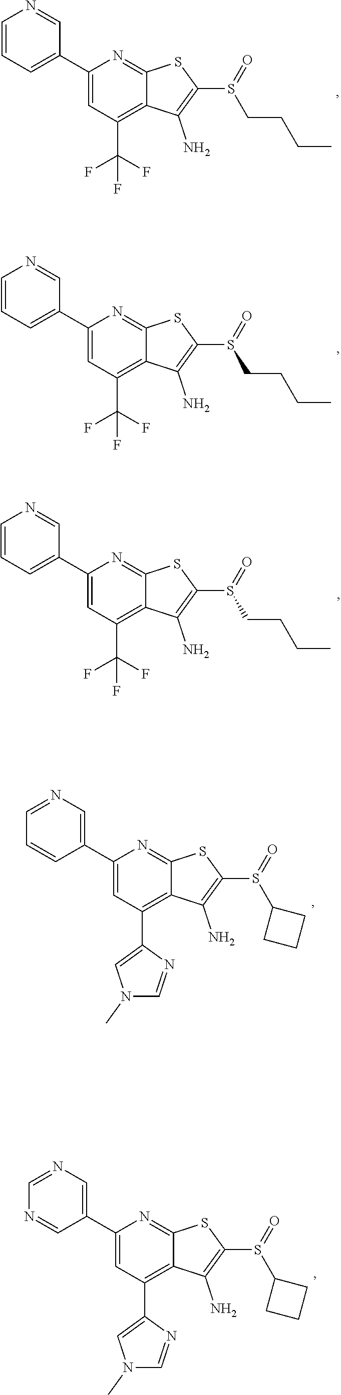

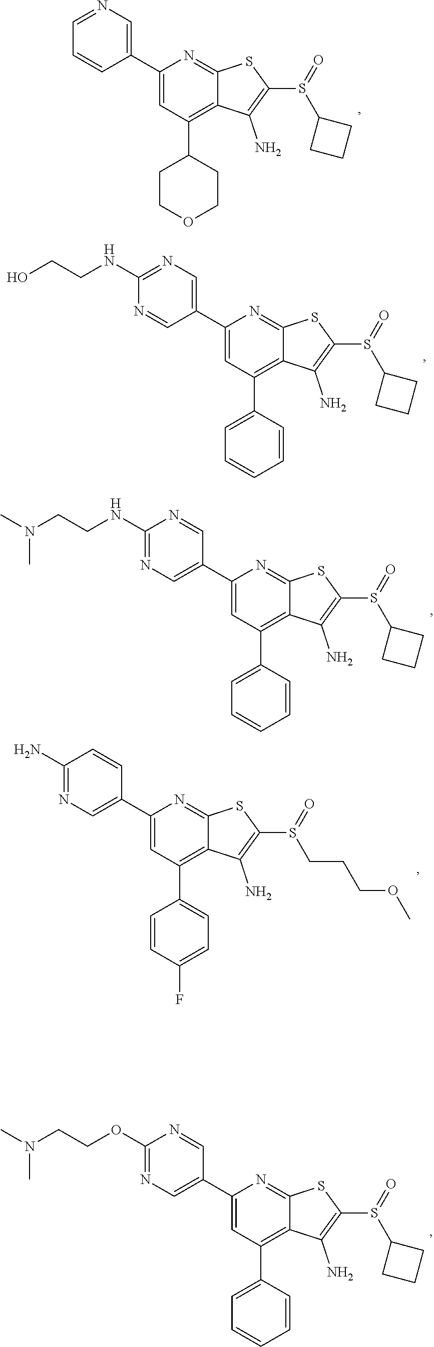

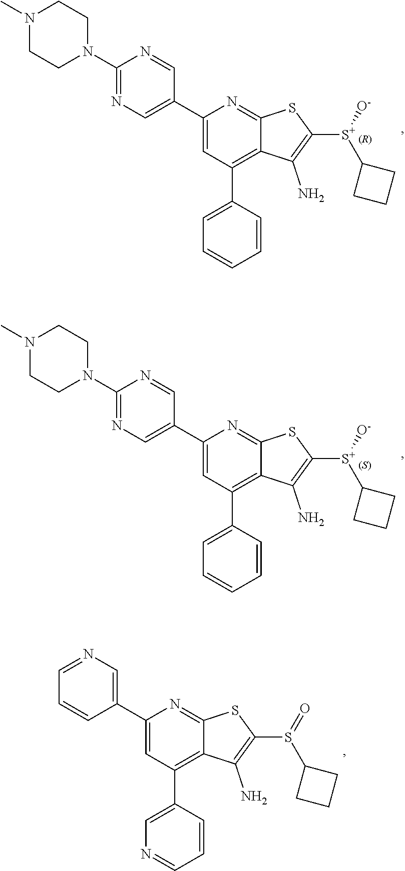

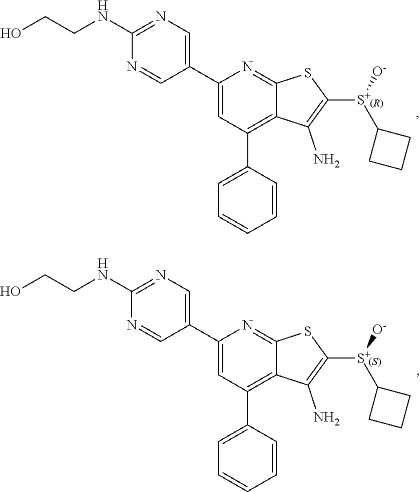

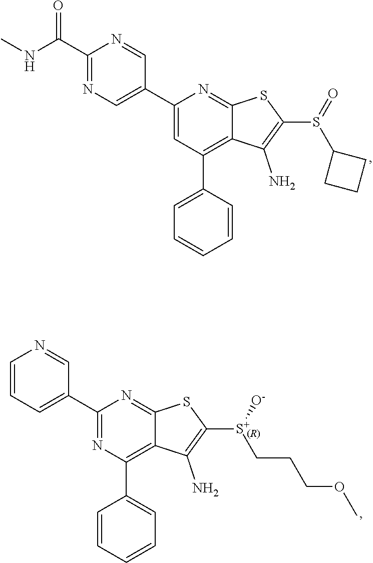

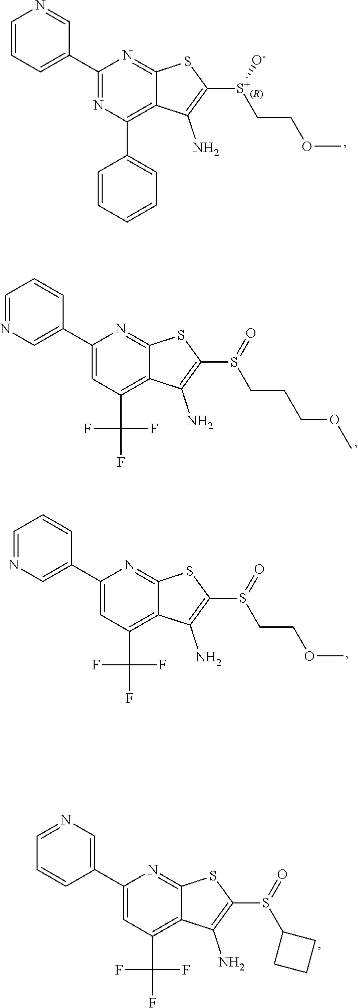

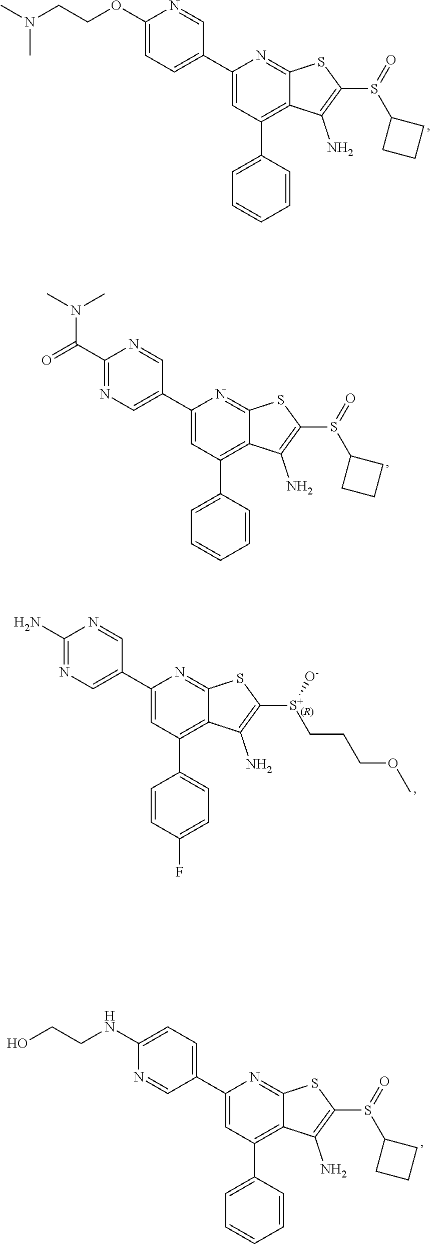

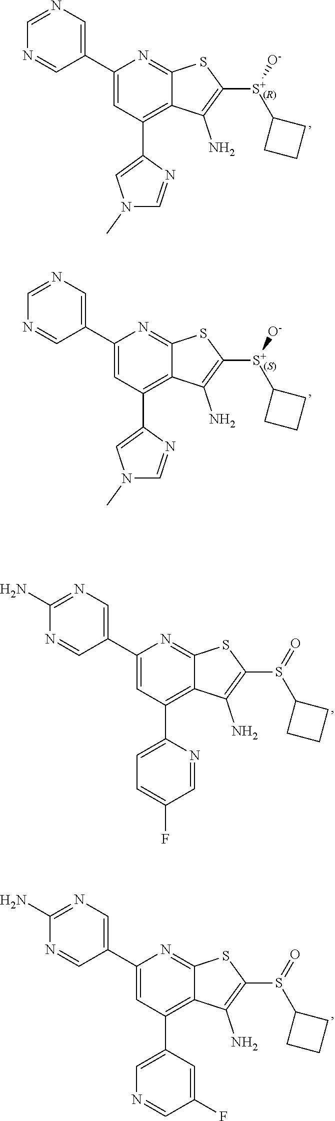

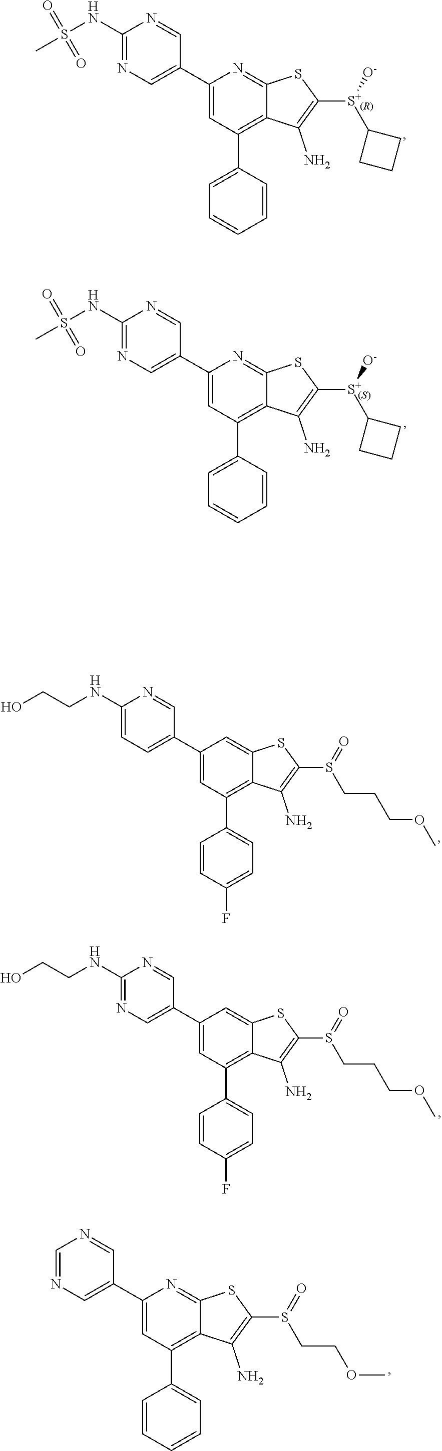

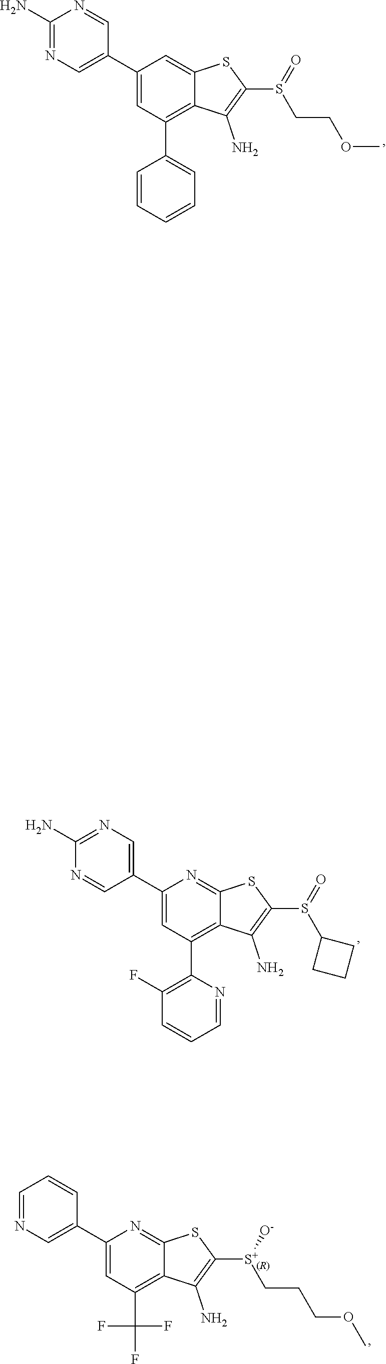

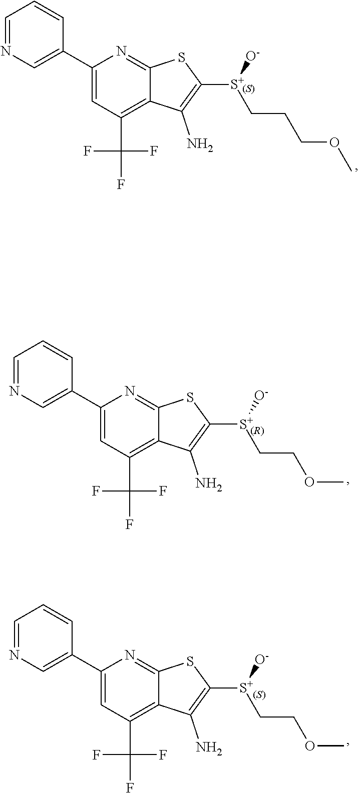

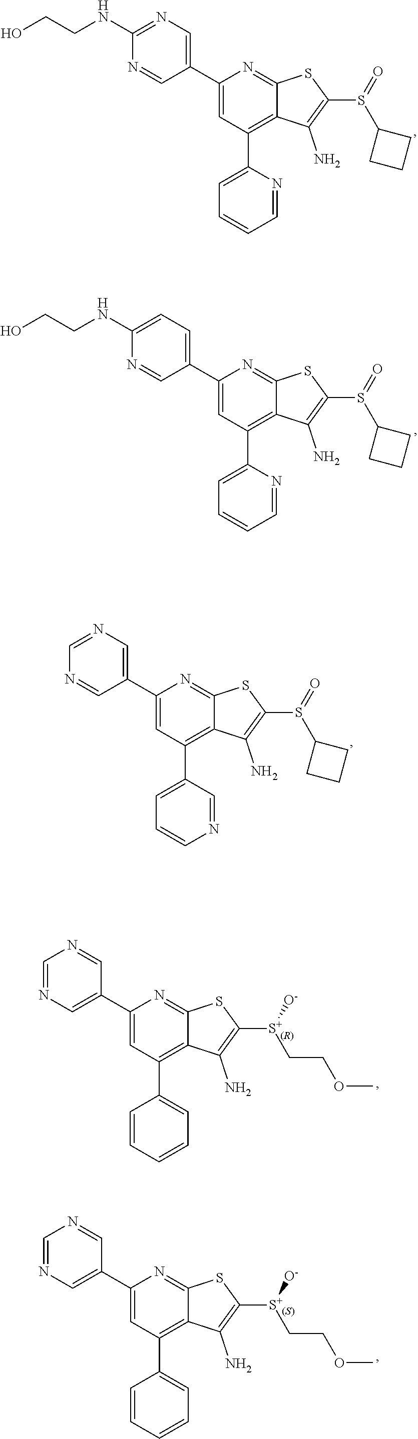

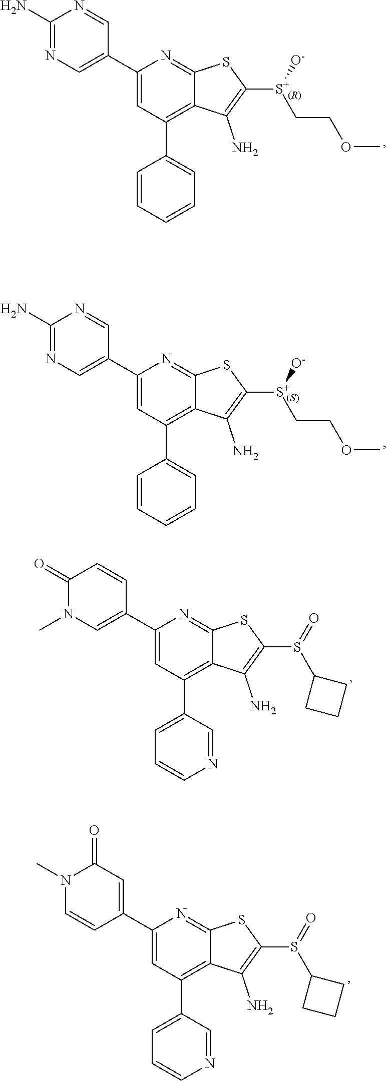

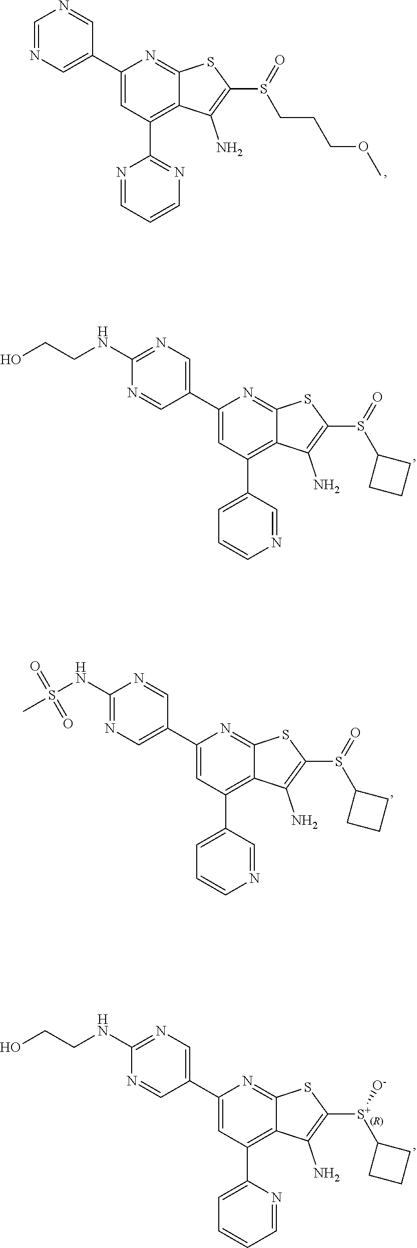

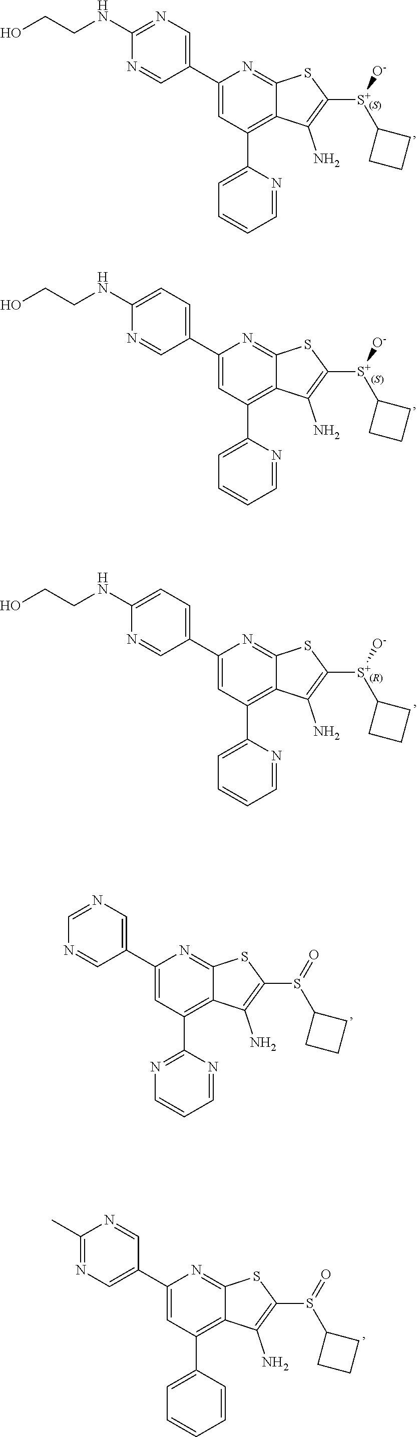

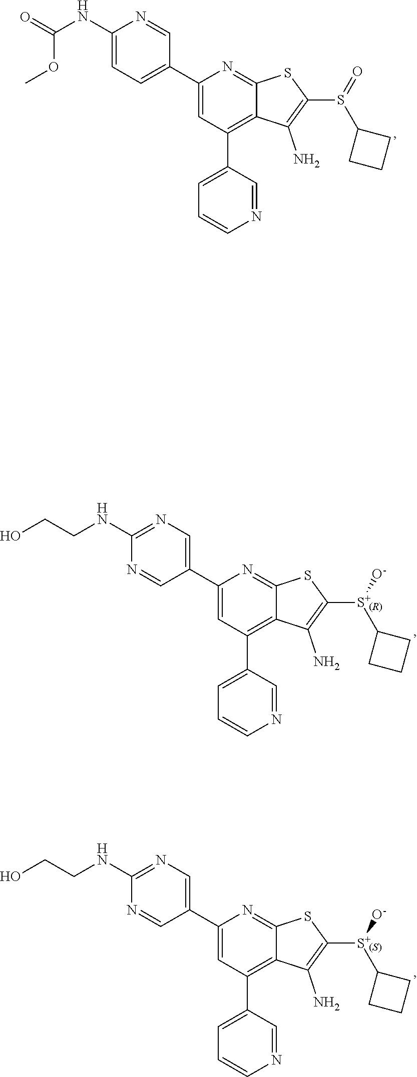

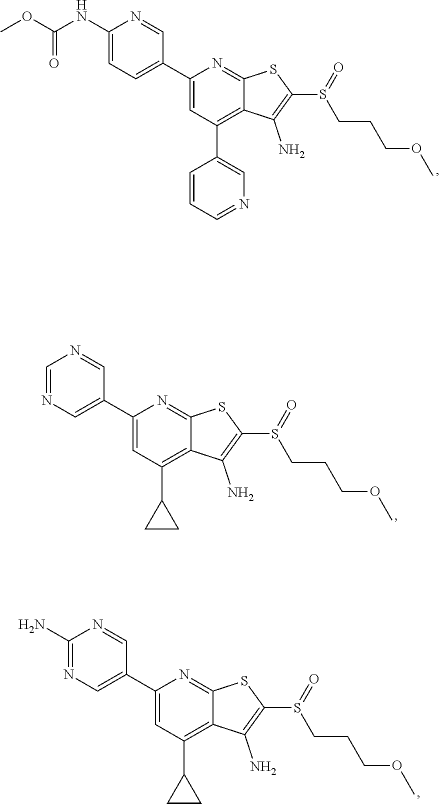

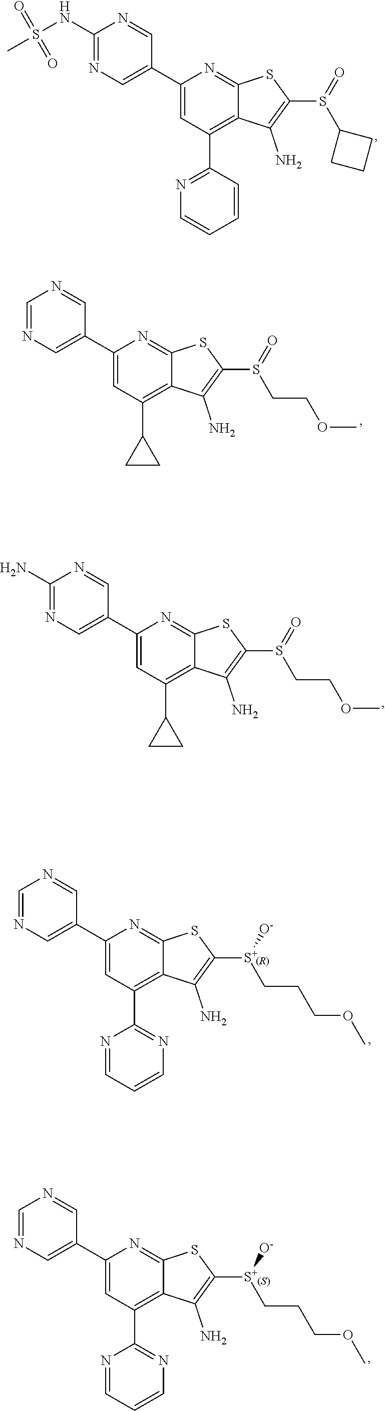

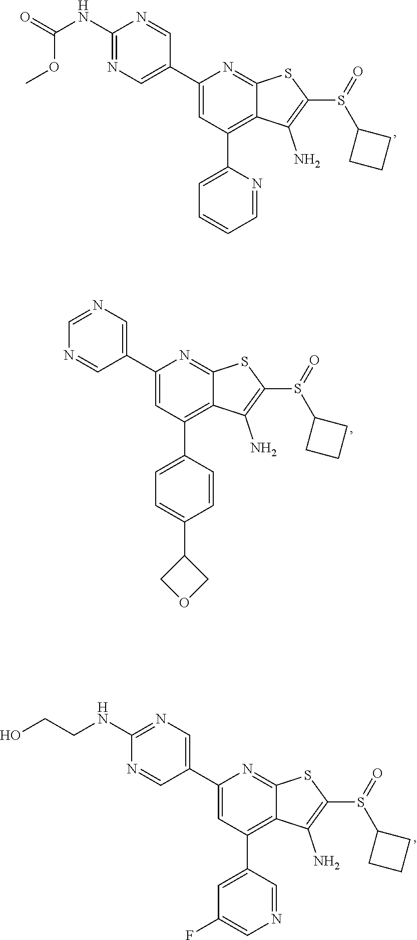 [SMILES: COC(=O)Nc1ncc(-c2cc(-c3ccccn3)c3c(N)c(S(=O)C4CCC4)sc3n2)cn1 ; "," ; Nc1c(S(=O)C2CCC2)sc2nc(-c3cncnc3)cc(-c3ccc(C4COC4)cc3)c12 ; "," ; Nc1c(S(=O)C2CCC2)sc2nc(-c3cnc(NCCO)nc3)cc(-c3cncc(F)c3)c12 ; ","]

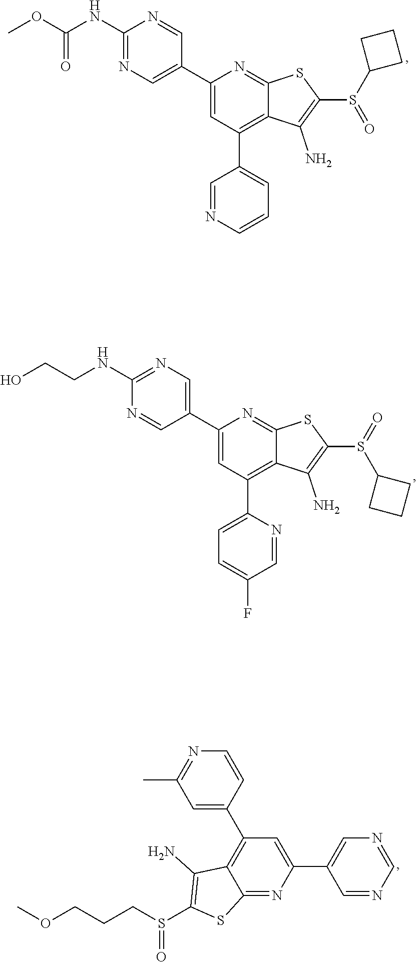 [SMILES: COC(=O)Nc1ncc(-c2cc(-c3cccnc3)c3c(N)c(S(=O)C4CCC4)sc3n2)cn1 ; "," ; Nc1c(S(=O)C2CCC2)sc2nc(-c3cnc(NCCO)nc3)cc(-c3ccc(F)cn3)c12 ; "," ; COCCCS(=O)c1sc2nc(-c3cncnc3)cc(-c3ccnc(C)c3)c2c1N ; ","]

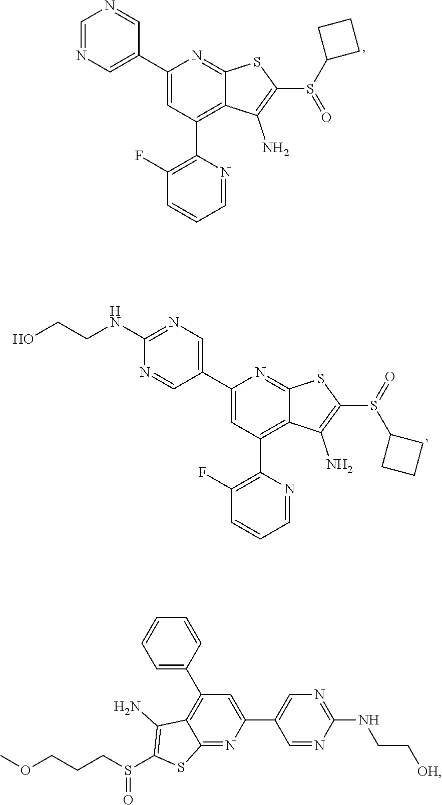 [SMILES: Nc1c(S(=O)C2CCC2)sc2nc(-c3cncnc3)cc(-c3ncccc3F)c12 ; "," ; Nc1c(S(=O)C2CCC2)sc2nc(-c3cnc(NCCO)nc3)cc(-c3ncccc3F)c12 ; "," ; COCCCS(=O)c1sc2nc(-c3cnc(NCCO)nc3)cc(-c3ccccc3)c2c1N]

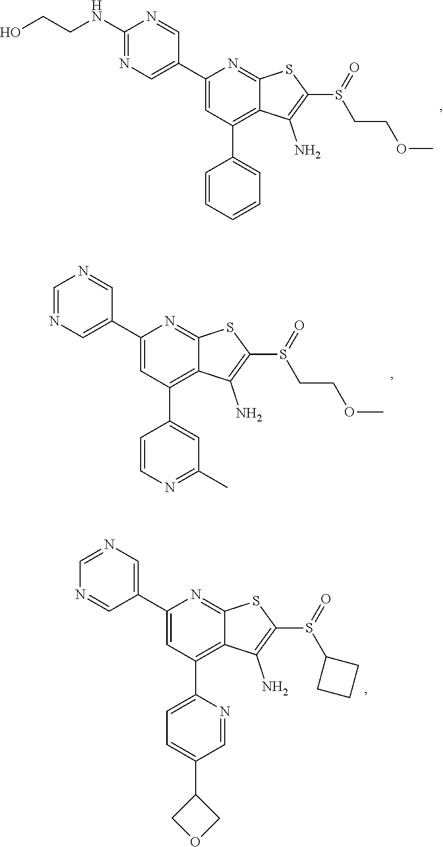 [SMILES: COCCS(=O)c1sc2nc(-c3cnc(NCCO)nc3)cc(-c3ccccc3)c2c1N ; "," ; COCCS(=O)c1sc2nc(-c3cncnc3)cc(-c3ccnc(C)c3)c2c1N ; "," ; Nc1c(S(=O)C2CCC2)sc2nc(-c3cncnc3)cc(-c3ccc(C4COC4)cn3)c12 ; ","]

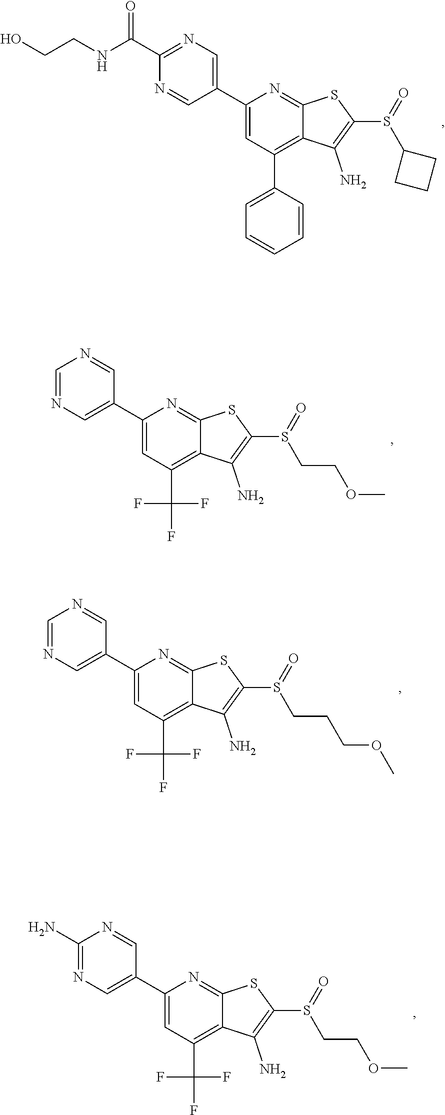

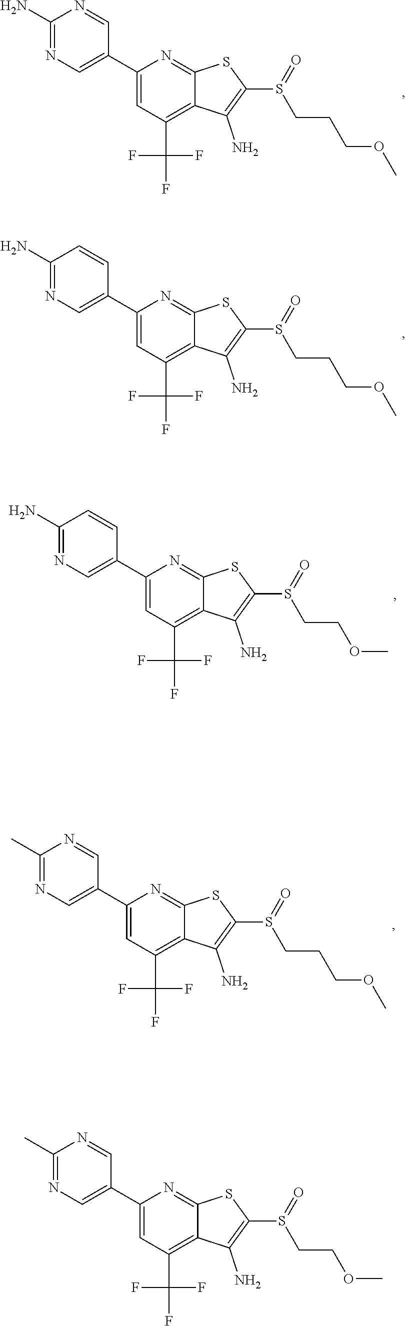

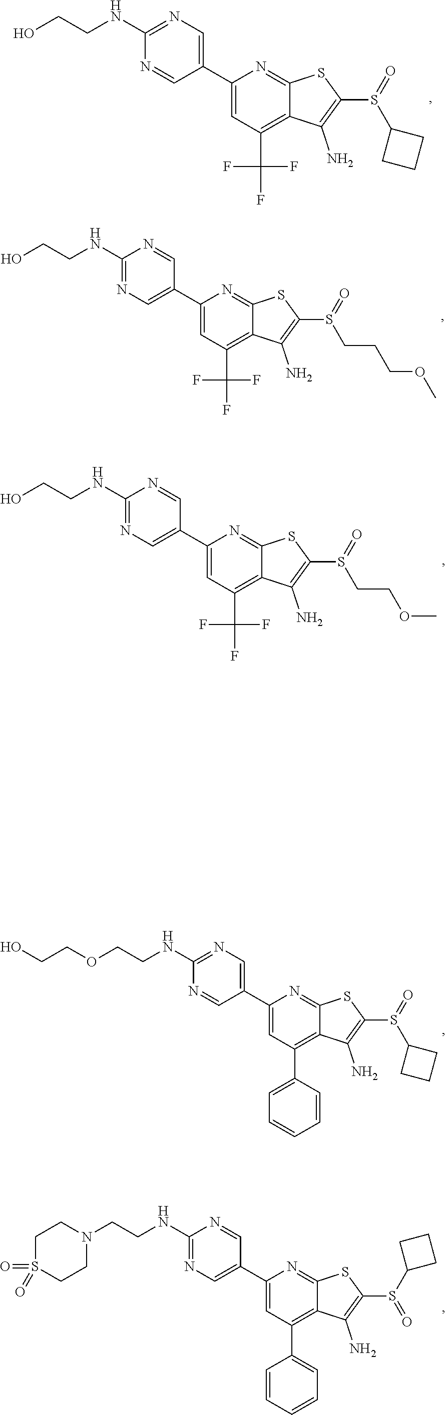

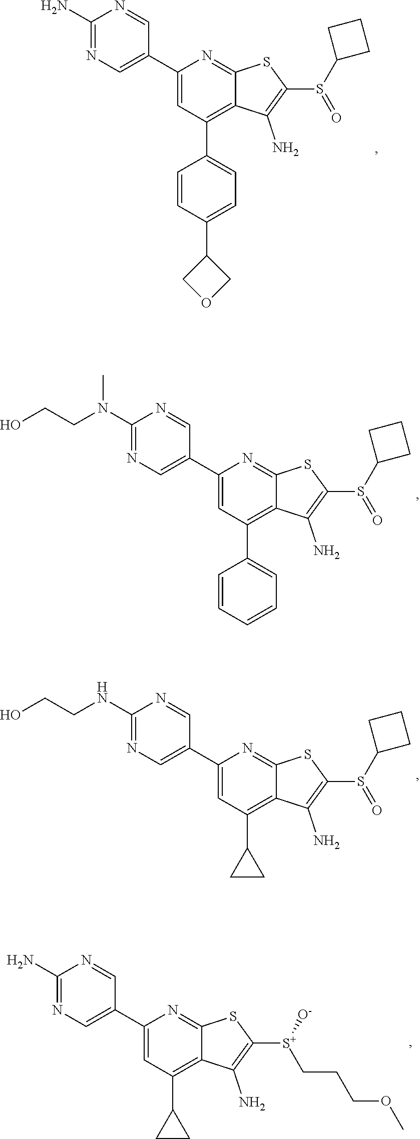

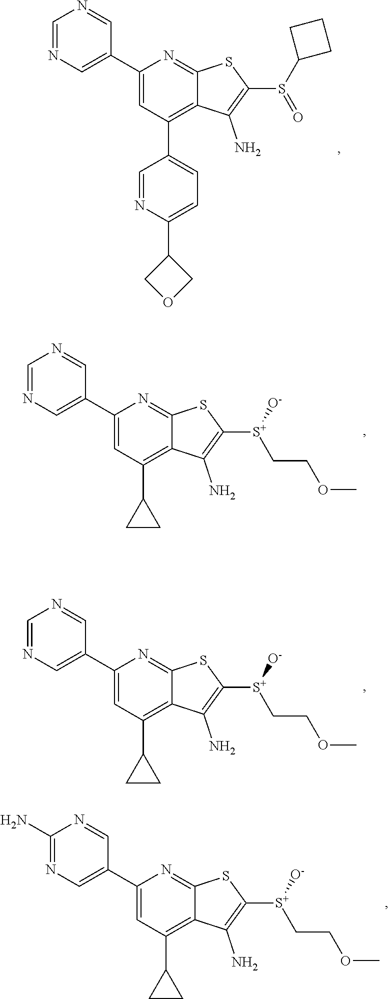

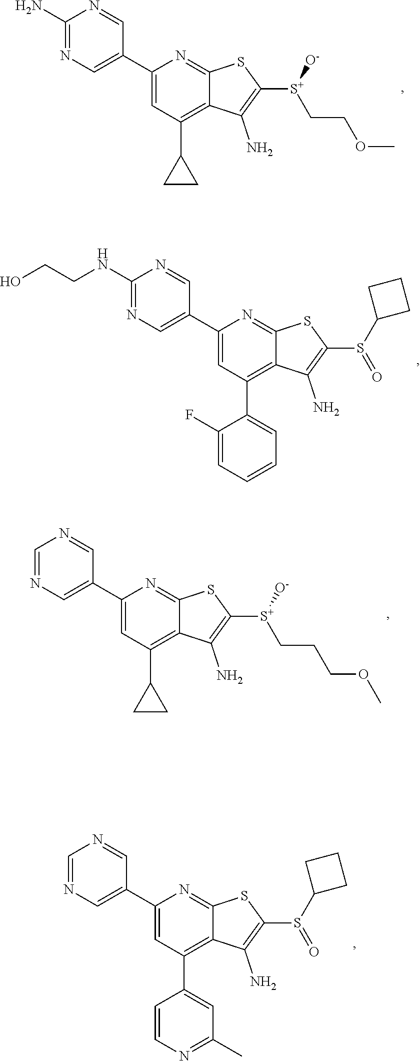

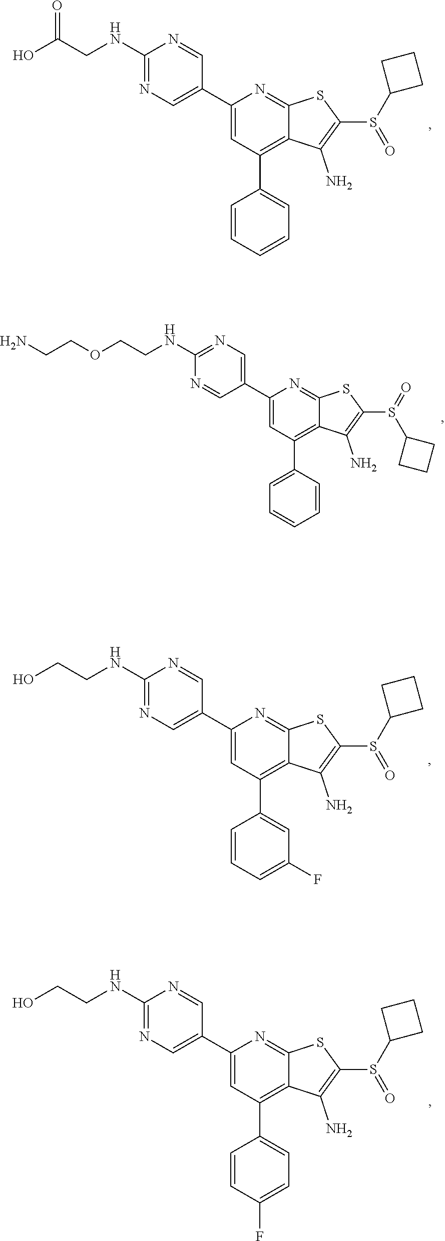

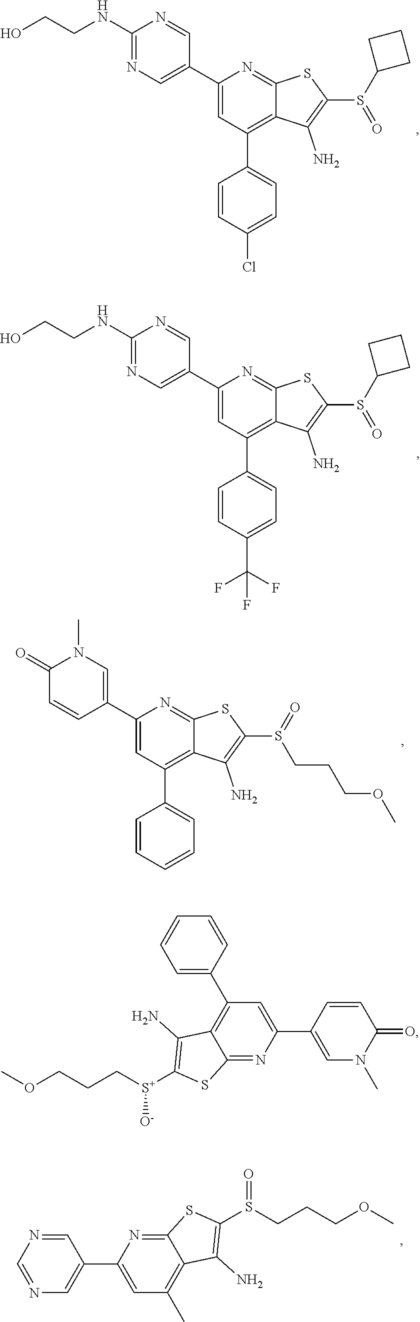

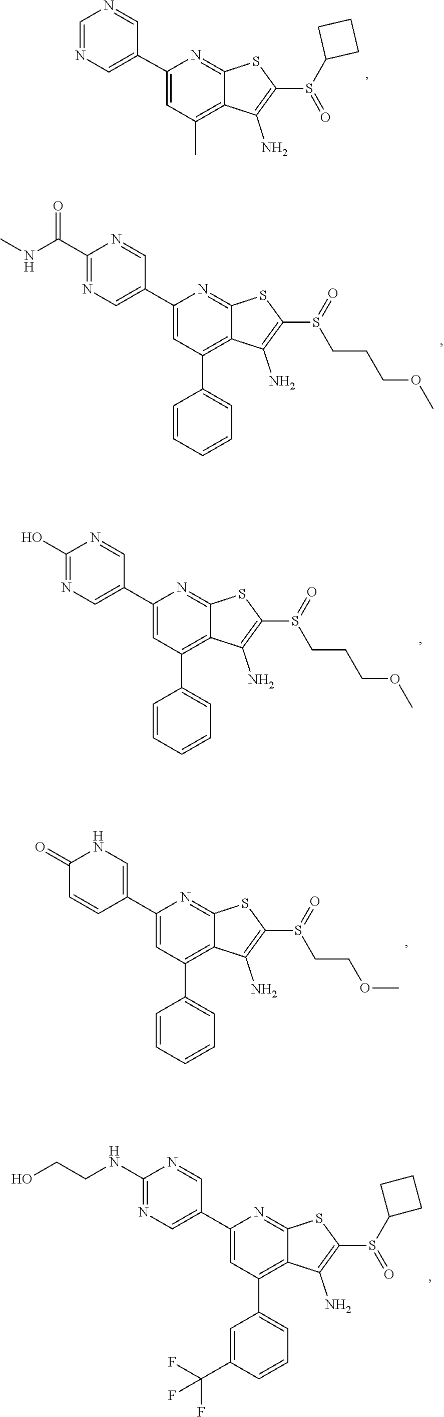

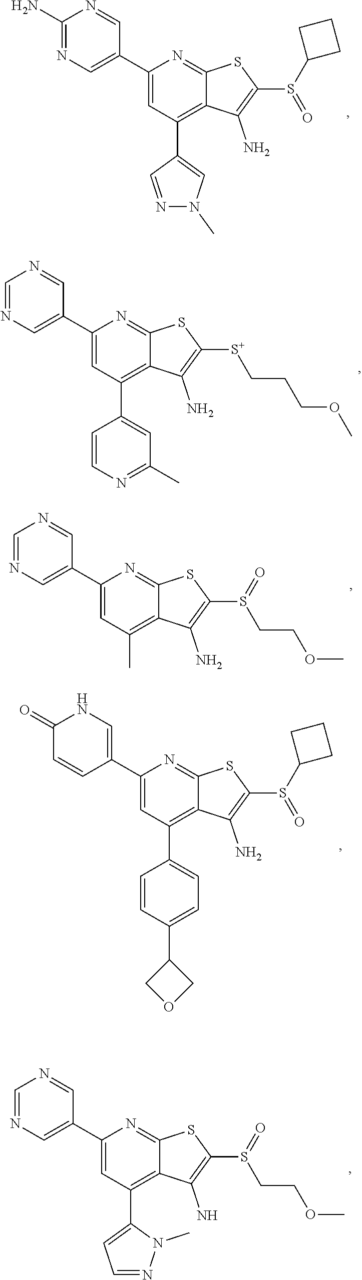

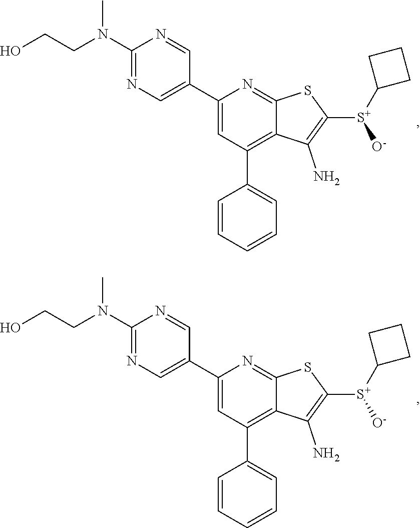

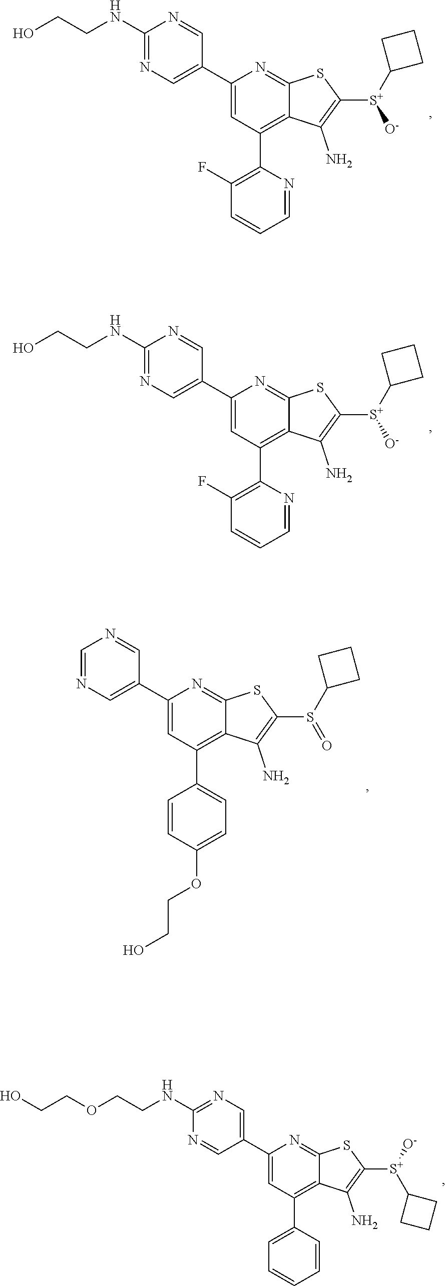

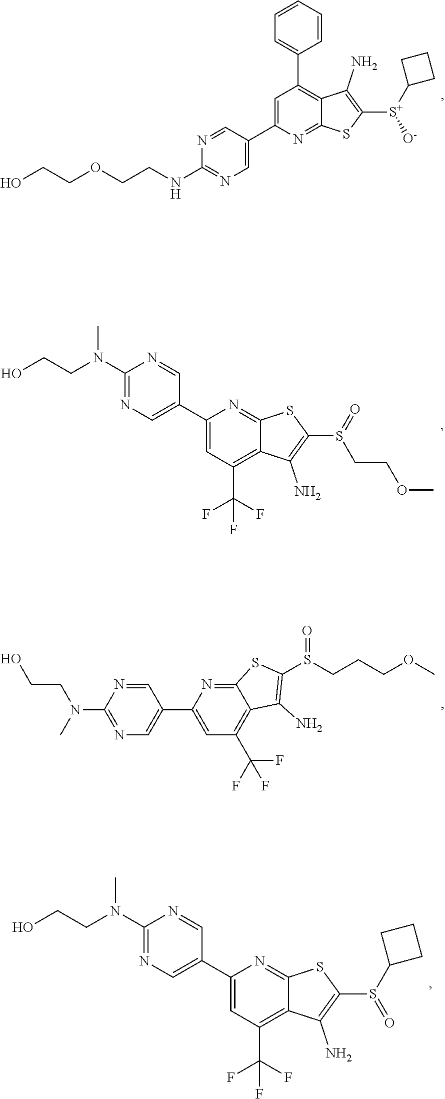

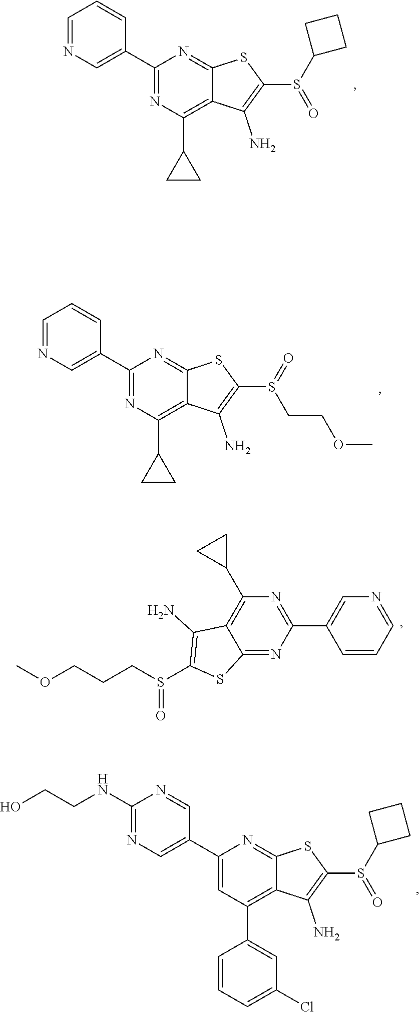

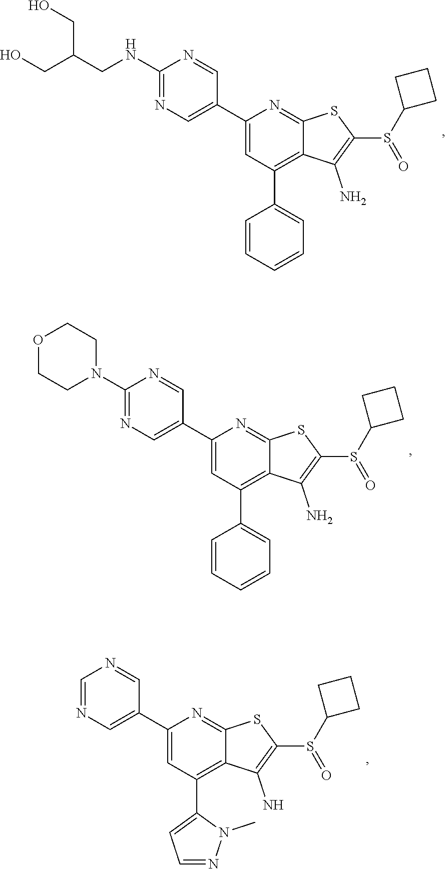 [SMILES: Nc1c(S(=O)C2CCC2)sc2nc(-c3cnc(NCC(CO)CO)nc3)cc(-c3ccccc3)c12 ; "," ; Nc1c(S(=O)C2CCC2)sc2nc(-c3cnc(N4CCOCC4)nc3)cc(-c3ccccc3)c12 ; "," ; Cn1nccc1-c1cc(-c2cncnc2)nc2sc(S(=O)C3CCC3)c(N)c12 ; ","]

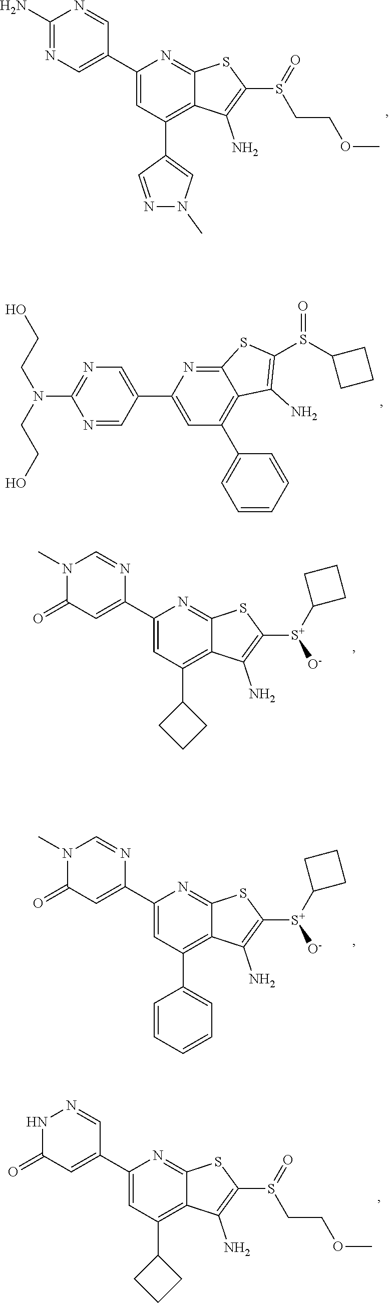

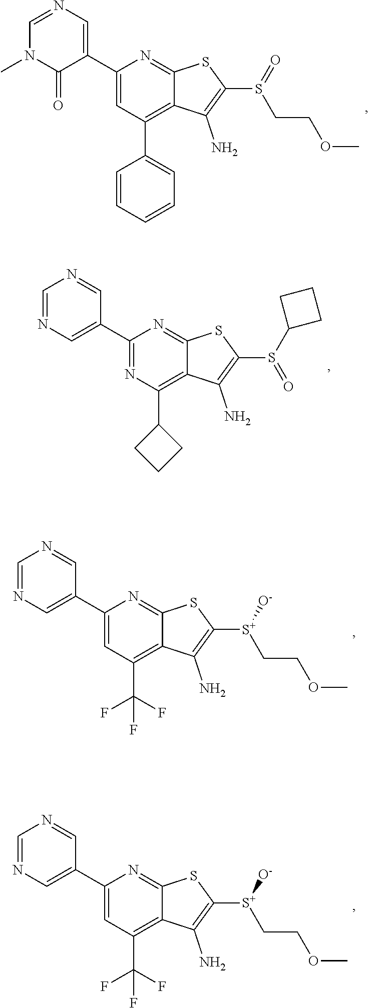

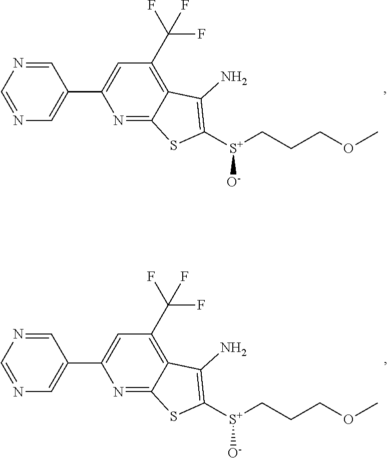 [SMILES: COCCC[S@@+]([O-])c1sc2nc(-c3cncnc3)cc(C(F)(F)F)c2c1N ; "," ; COCCC[S@+]([O-])c1sc2nc(-c3cncnc3)cc(C(F)(F)F)c2c1N ; ","]

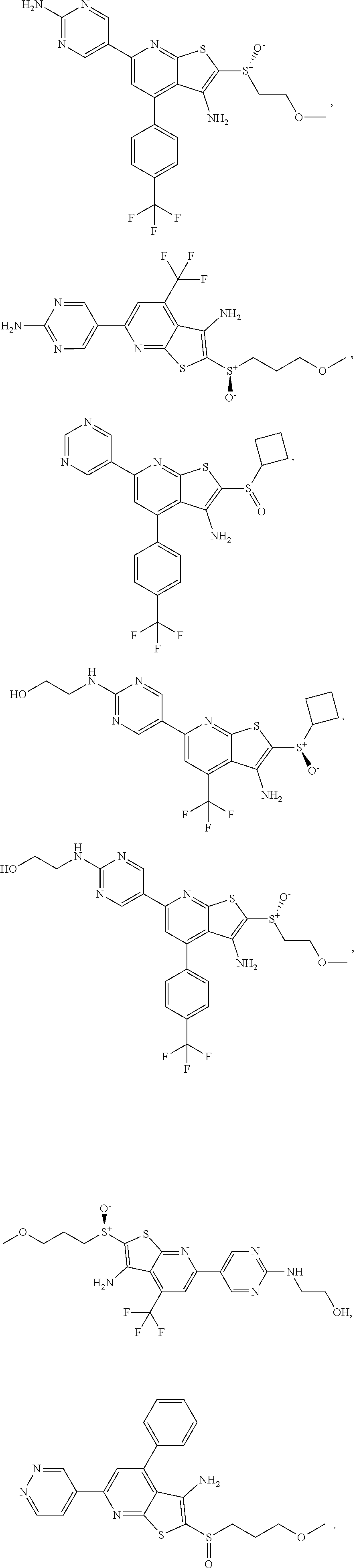

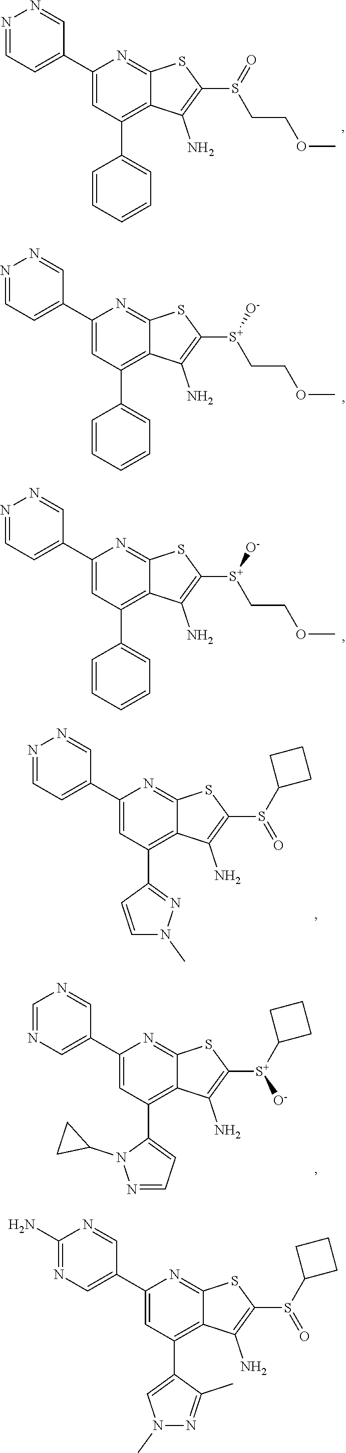 [SMILES: COCCS(=O)c1sc2nc(-c3ccnnc3)cc(-c3ccccc3)c2c1N ; COCC[S@@+]([O-])c1sc2nc(-c3ccnnc3)cc(-c3ccccc3)c2c1N ; COCC[S@+]([O-])c1sc2nc(-c3ccnnc3)cc(-c3ccccc3)c2c1N ; Cn1ccc(-c2cc(-c3ccnnc3)nc3sc(S(=O)C4CCC4)c(N)c23)n1 ; Nc1c([S@+]([O-])C2CCC2)sc2nc(-c3cncnc3)cc(-c3ccnn3C3CC3)c12 ; Cc1nn(C)cc1-c1cc(-c2cnc(N)nc2)nc2sc(S(=O)C3CCC3)c(N)c12]

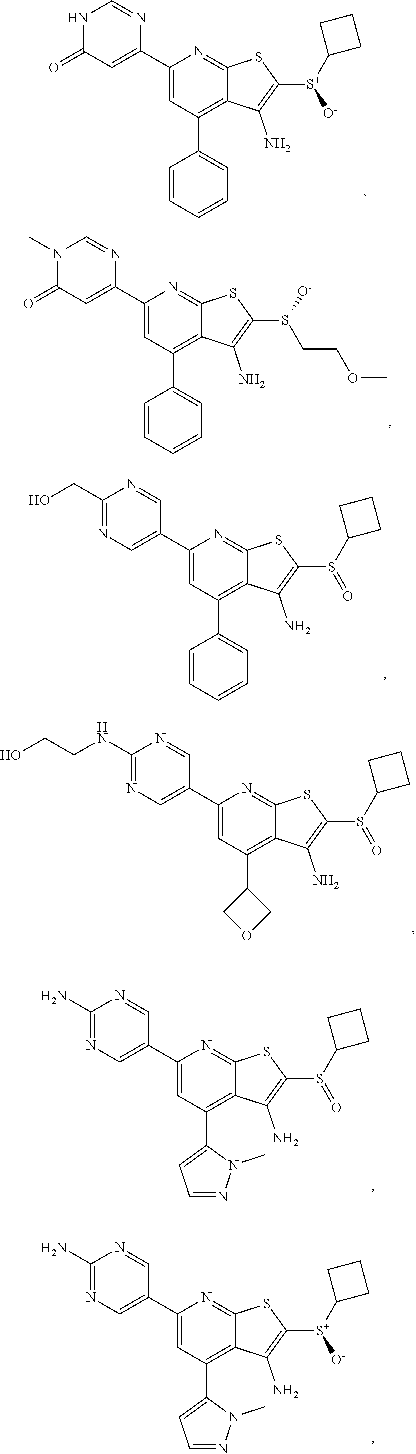 [SMILES: Nc1c([S@+]([O-])C2CCC2)sc2nc(-c3cc(=O)[nH]cn3)cc(-c3ccccc3)c12 ; COCC[S@@+]([O-])c1sc2nc(-c3cc(=O)n(C)cn3)cc(-c3ccccc3)c2c1N ; Nc1c(S(=O)C2CCC2)sc2nc(-c3cnc(CO)nc3)cc(-c3ccccc3)c12 ; Nc1c(S(=O)C2CCC2)sc2nc(-c3cnc(NCCO)nc3)cc(C3COC3)c12 ; Cn1nccc1-c1cc(-c2cnc(N)nc2)nc2sc(S(=O)C3CCC3)c(N)c12 ; Cn1nccc1-c1cc(-c2cnc(N)nc2)nc2sc([S@+]([O-])C3CCC3)c(N)c12]

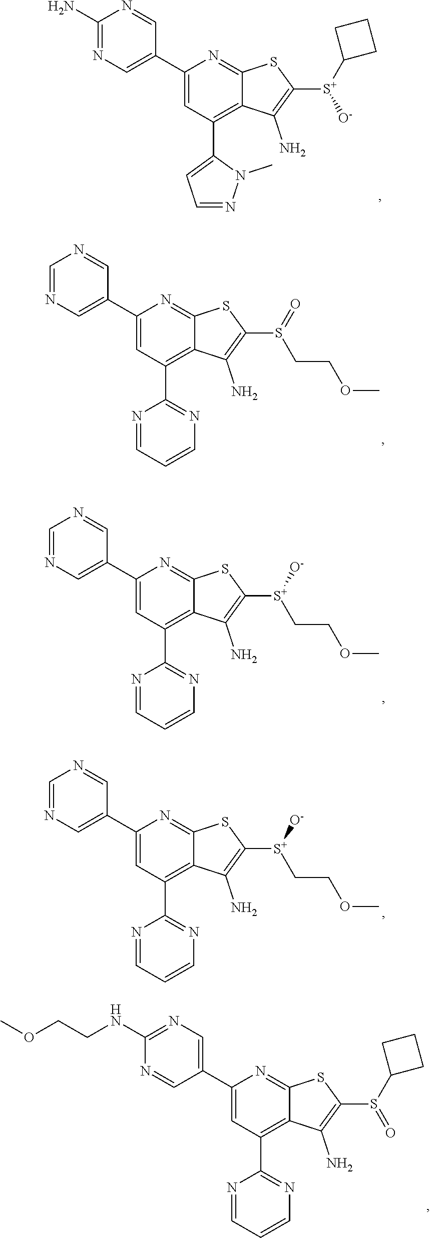

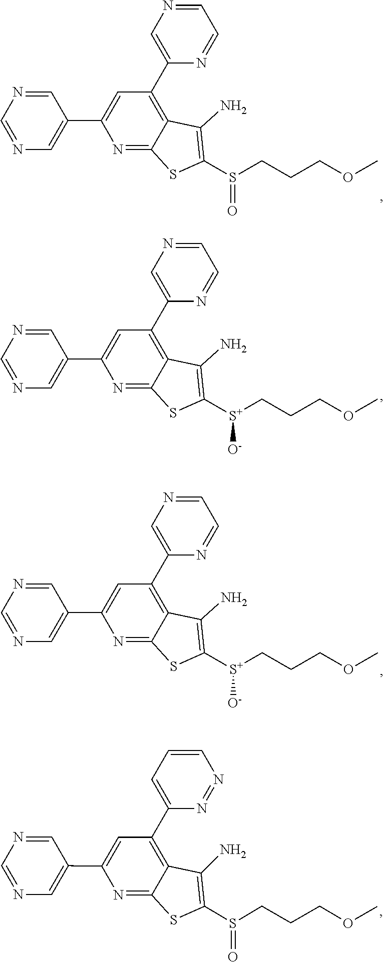

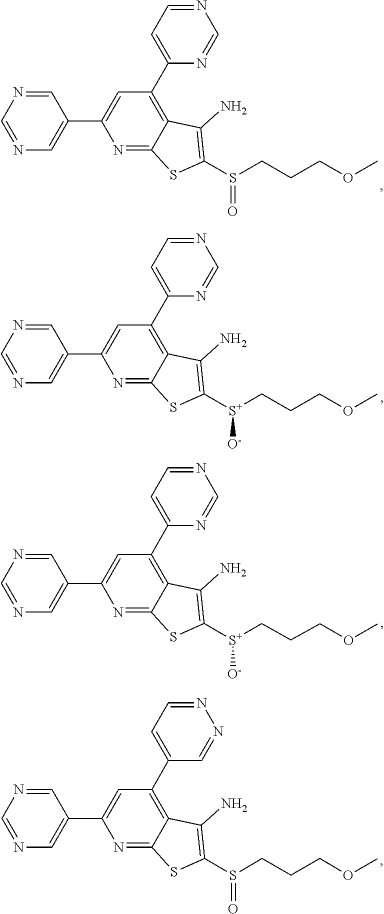

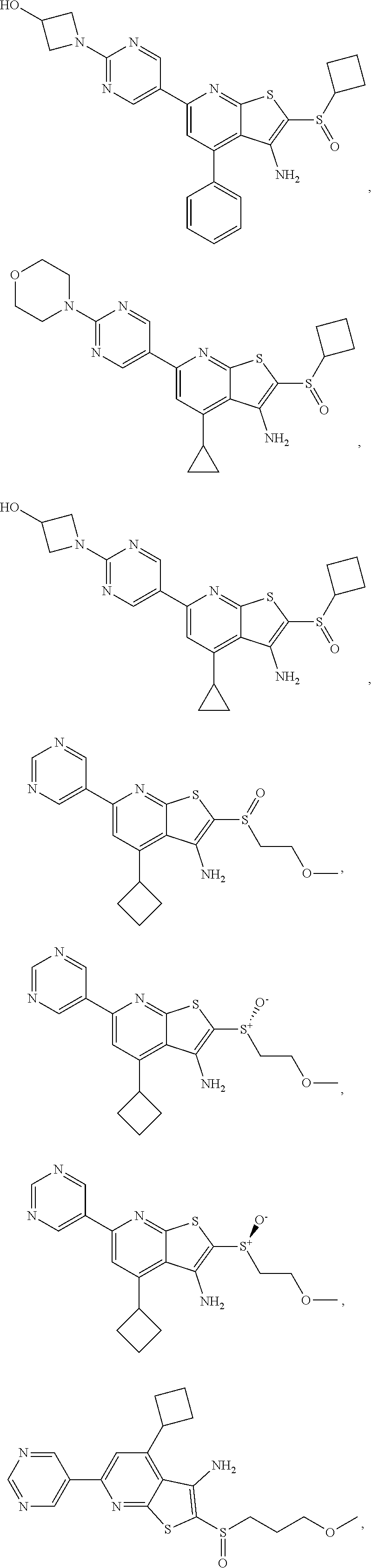

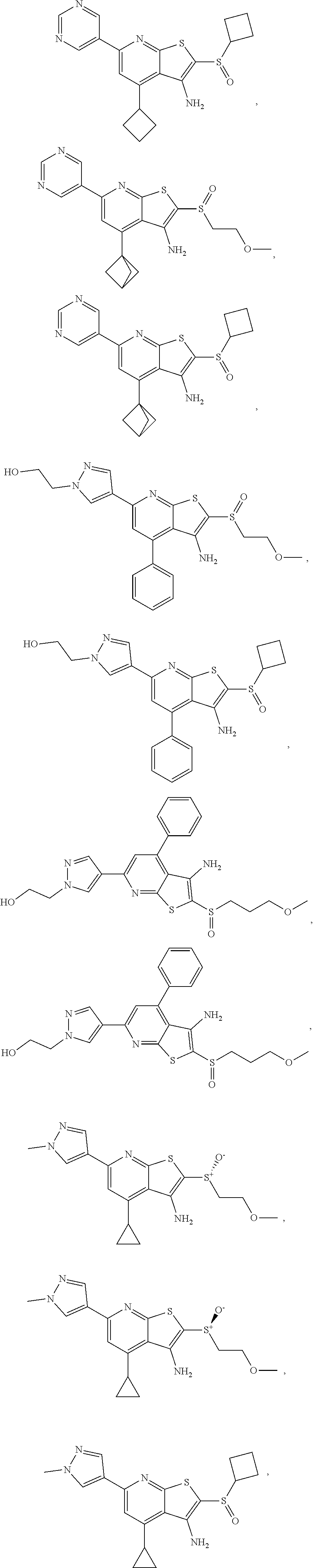

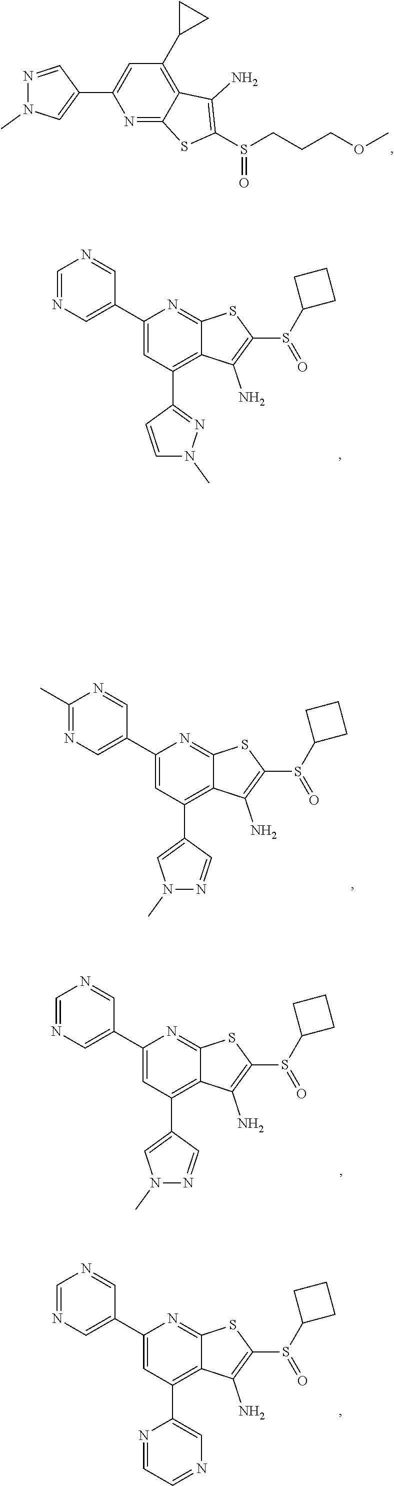

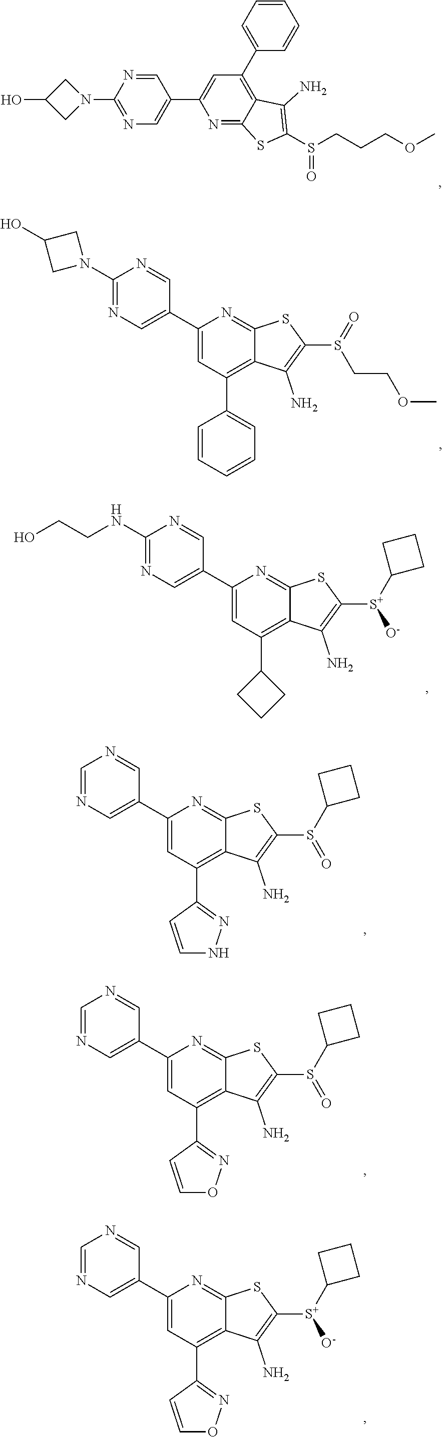 [SMILES: COCCCS(=O)c1sc2nc(-c3cnc(N4CC(O)C4)nc3)cc(-c3ccccc3)c2c1N ; COCCS(=O)c1sc2nc(-c3cnc(N4CC(O)C4)nc3)cc(-c3ccccc3)c2c1N ; Nc1c([S@+]([O-])C2CCC2)sc2nc(-c3cnc(NCCO)nc3)cc(C3CCC3)c12 ; Nc1c(S(=O)C2CCC2)sc2nc(-c3cncnc3)cc(-c3cc[nH]n3)c12 ; Nc1c(S(=O)C2CCC2)sc2nc(-c3cncnc3)cc(-c3ccon3)c12 ; Nc1c([S@+]([O-])C2CCC2)sc2nc(-c3cncnc3)cc(-c3ccon3)c12]

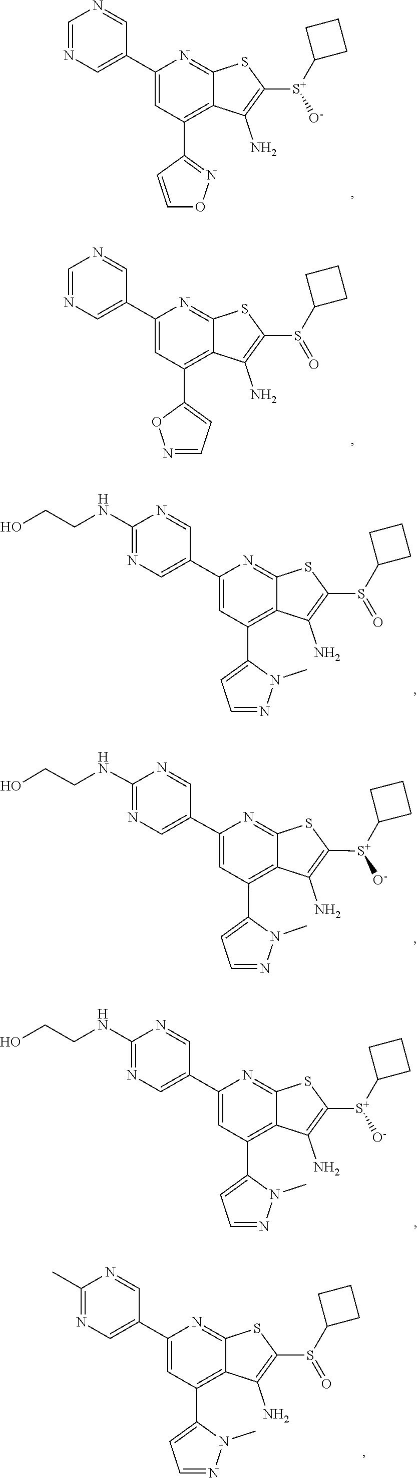 [SMILES: Nc1c([S@@+]([O-])C2CCC2)sc2nc(-c3cncnc3)cc(-c3ccon3)c12 ; Nc1c(S(=O)C2CCC2)sc2nc(-c3cncnc3)cc(-c3ccno3)c12 ; Cn1nccc1-c1cc(-c2cnc(NCCO)nc2)nc2sc(S(=O)C3CCC3)c(N)c12 ; Cn1nccc1-c1cc(-c2cnc(NCCO)nc2)nc2sc([S@+]([O-])C3CCC3)c(N)c12 ; Cn1nccc1-c1cc(-c2cnc(NCCO)nc2)nc2sc([S@@+]([O-])C3CCC3)c(N)c12 ; Cc1ncc(-c2cc(-c3ccnn3C)c3c(N)c(S(=O)C4CCC4)sc3n2)cn1]

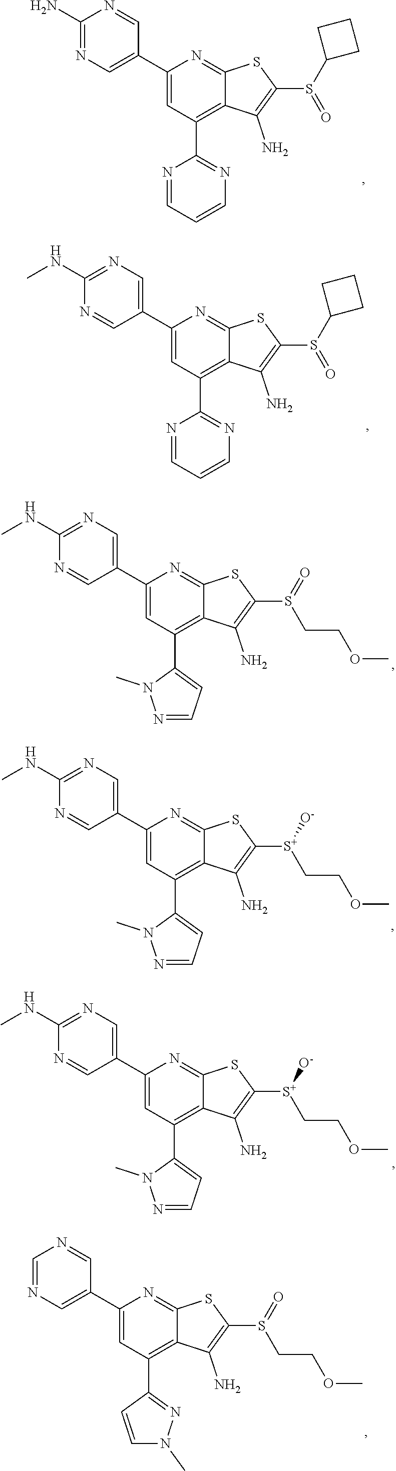

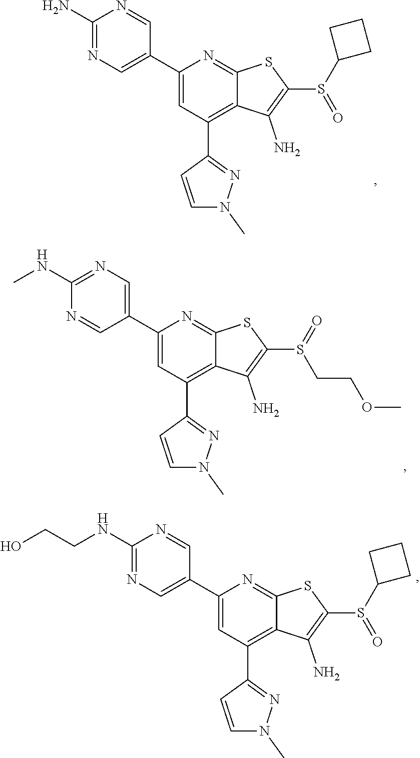

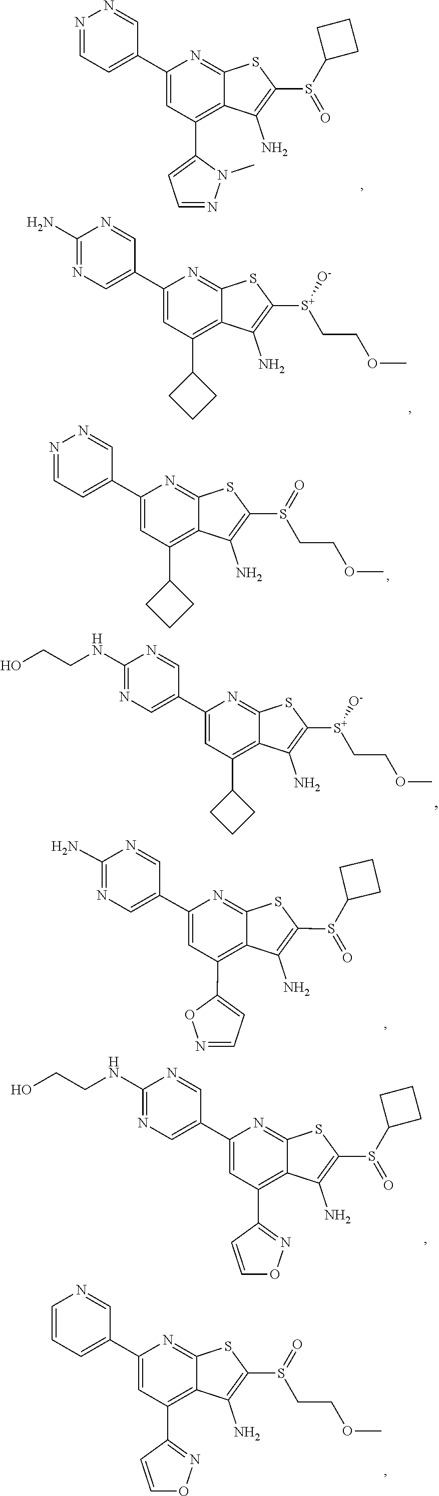

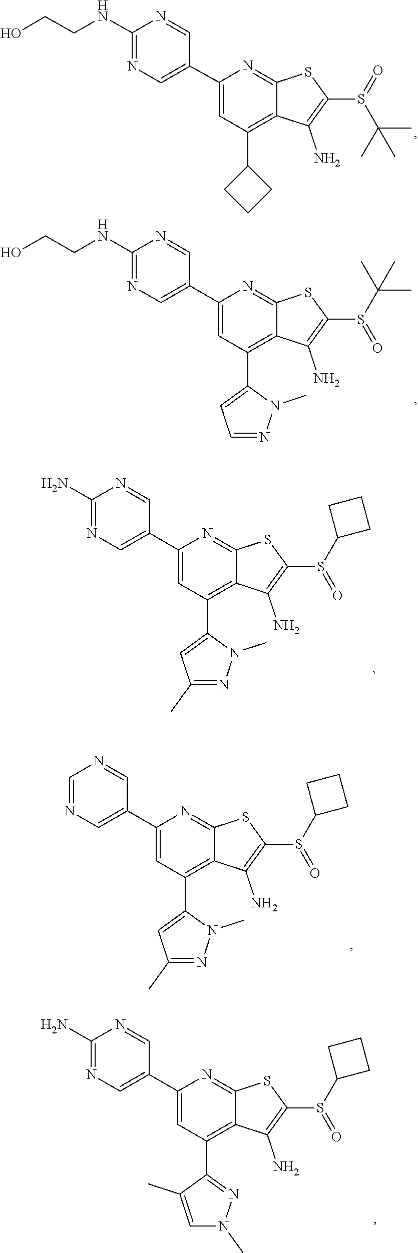

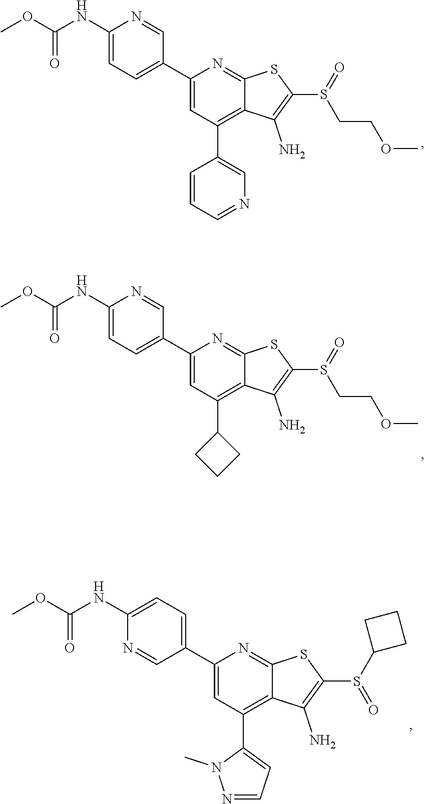

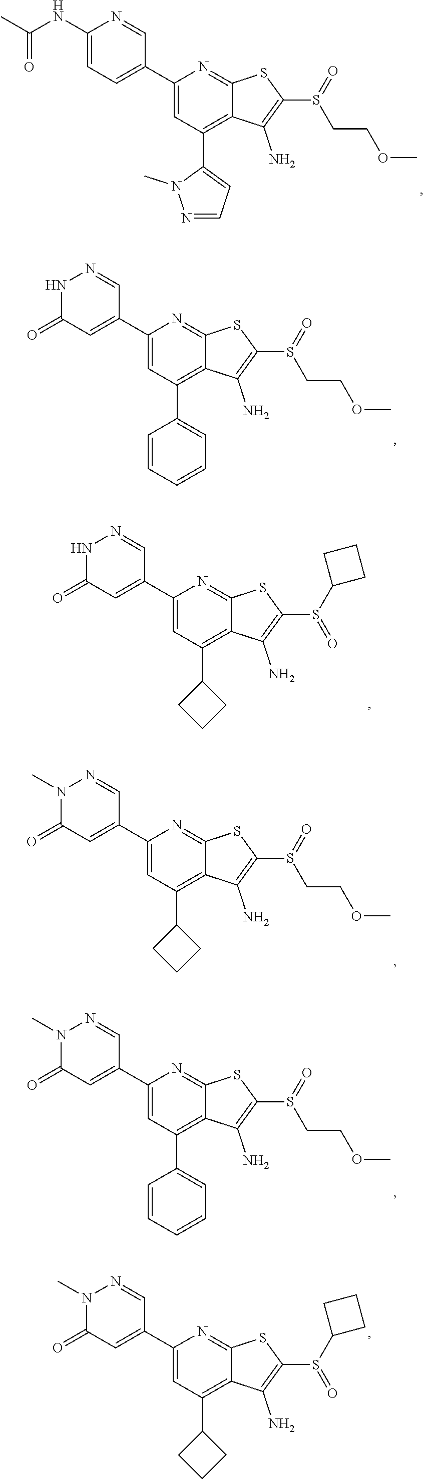 [SMILES: COCCS(=O)c1sc2nc(-c3ccc(NC(C)=O)nc3)cc(-c3ccnn3C)c2c1N ; COCCS(=O)c1sc2nc(-c3cn[nH]c(=O)c3)cc(-c3ccccc3)c2c1N ; Nc1c(S(=O)C2CCC2)sc2nc(-c3cn[nH]c(=O)c3)cc(C3CCC3)c12 ; COCCS(=O)c1sc2nc(-c3cnn(C)c(=O)c3)cc(C3CCC3)c2c1N ; COCCS(=O)c1sc2nc(-c3cnn(C)c(=O)c3)cc(-c3ccccc3)c2c1N ; Cn1ncc(-c2cc(C3CCC3)c3c(N)c(S(=O)C4CCC4)sc3n2)cc1=O]

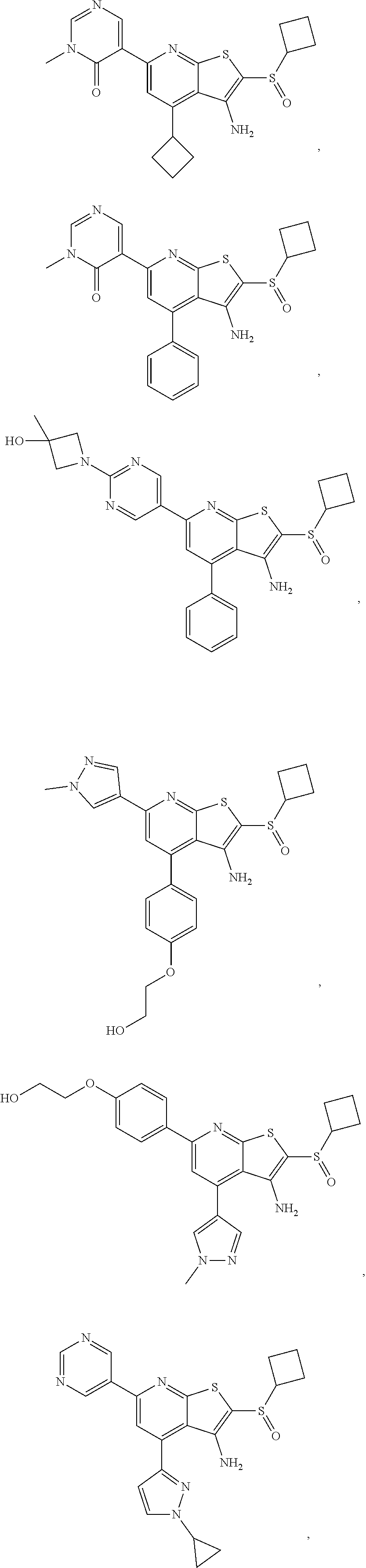 [SMILES: Cn1cncc(-c2cc(C3CCC3)c3c(N)c(S(=O)C4CCC4)sc3n2)c1=O ; Cn1cncc(-c2cc(-c3ccccc3)c3c(N)c(S(=O)C4CCC4)sc3n2)c1=O ; CC1(O)CN(c2ncc(-c3cc(-c4ccccc4)c4c(N)c(S(=O)C5CCC5)sc4n3)cn2)C1 ; Cn1cc(-c2cc(-c3ccc(OCCO)cc3)c3c(N)c(S(=O)C4CCC4)sc3n2)cn1 ; Cn1cc(-c2cc(-c3ccc(OCCO)cc3)nc3sc(S(=O)C4CCC4)c(N)c23)cn1 ; Nc1c(S(=O)C2CCC2)sc2nc(-c3cncnc3)cc(-c3ccn(C4CC4)n3)c12]

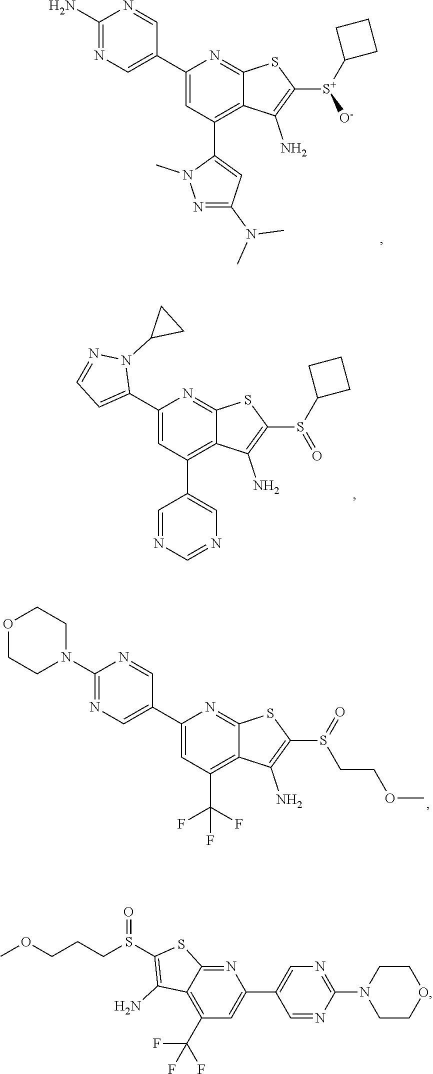

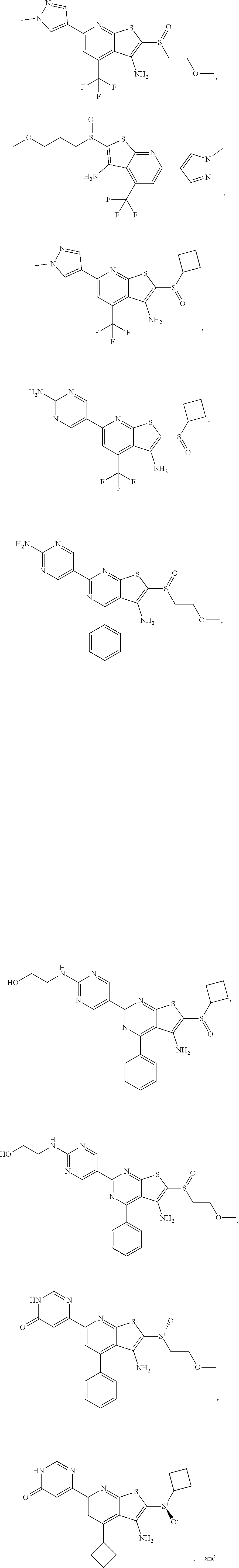

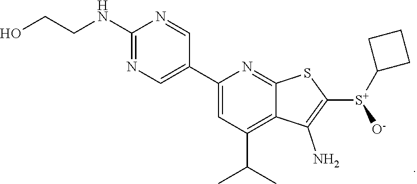 [SMILES: CC(C)c1cc(-c2cnc(NCCO)nc2)nc2sc([S@+]([O-])C3CCC3)c(N)c12]

16. The compound of claim 1.

17. A pharmaceutical composition comprising the compound, solvate, or salt of claim 1 and a pharmaceutically acceptable excipient or a carrier.

18. A method of treating inflammatory bowel disease, the method comprising administering to a human subject in need of treatment a therapeutic amount of the compound of claim 1.

19. A method of:
(i) treating a subject in need of cell therapy comprising administering to the subject a therapeutically effective amount of a preparation comprising human hematopoietic stem cells and the compound, salt, or solvate of claim 1 and/or a therapeutic composition comprising human hematopoietic stem cells and the compound, salt, or solvate of claim 1; or
(ii) treating a subject having at least one symptom associated with an ischemic tissue or a tissue damaged by ischemia comprising administering to the subject a therapeutically effective amount of a preparation comprising the compound, salt, or solvate of claim 1 and/or a therapeutic composition comprising human hematopoietic stem cells and the compound, salt, or solvate of claim 1; or
(iii) increasing neutrophils in a subject in need thereof, the method comprising administering to the subject the compound, salt, or solvate of claim 1; or
(iv) increasing numbers of and/or mobilizing peripheral blood hematopoietic stem cells in a subject in need thereof, the method comprising administering to the subject the compound, salt, or solvate of claim 1; or
(v) increasing numbers of hematopoietic stem cells in blood or bone marrow, the method comprising: administering to blood or bone marrow of the subject the compound, salt, or solvate of claim 1; or
(vi) treating or preventing a fibrotic disease, disorder or condition in a subject in need thereof, the method comprising administering to the subject a therapeutically effective amount of the compound, salt, or solvate of claim 1; or
(vii) treating intestinal, gastrointestinal, or bowel disorders in a subject in need thereof, the method comprising administering to the subject a therapeutically effective amount of the compound, salt, or solvate of claim 1 alone or in combination with a corticosteroid and/or a tumor necrosis factor a (TNFα) inhibitor; or
(viii) treating intestinal, gastrointestinal, or bowel disorders in a subject in need thereof, the method comprising administering to the subject therapeutically effective amounts of the compound, salt, or solvate of claim 1 and a corticosteroid; or
(ix) treating inflammation and/or reducing the activity of the immune system in a subject in need thereof, the method comprising administering to the subject a therapeutically effective amount of the compound, salt, or solvate of claim 1 and a corticosteroid; or
(x) the treatment of glucocorticoid insensitivity, restoring corticosteroid sensitivity, enhancing glucocorticoid sensitivity or reversing the glucocorticoid insensitivity in a subject experiencing corticosteroid dependence or corticoid resistance or unresponsiveness or intolerance to corticosteroids, comprising administering a pharmaceutical composition comprising the compound, salt, or solvate of claim 1 in combination with a corticosteroid to the subject exhibiting one or more glucocorticoid insensitivity related conditions, wherein the glucocorticoid insensitivity related conditions comprise a range of immune-inflammatory disorders/diseases treated with steroids when the therapy fails to achieve disease control or is not effective or intolerant or dependent to corticosteroids, and combinations thereof.
